(19) United States
(12) Patent Application Publication
(10) Pub. No.: US 2025/0221145 A1
(43) Pub. Date: Jul. 3, 2025

(54) HETEROCYCLIC COMPOUND, ORGANIC LIGHT-EMITTING DEVICE COMPRISING SAME, AND COMPOSITION COMPRISING SAME FOR FORMING ORGANIC LAYER

(71) Applicant: LT MATERIALS CO., LTD., Yongin-si, Gyeonggi-do (KR)

(72) Inventors: Gi-Back LEE, Yongin-si (KR); Jun-Tae MO, Yongin-si (KR); Dong-Jun KIM, Yongin-si (KR)

(73) Assignee: LT MATERIALS CO., LTD., Yongin-si, Gyeonggi-do (KR)

(21) Appl. No.: 18/848,371

(22) PCT Filed: Mar. 14, 2023
(86) PCT No.: PCT/KR2023/003415

§ 371 Date: Sep. 18, 2024

(30) Foreign Application Priority Data
Mar. 31, 2022 (KR) 10-2022-0040365

(51) Int. Cl. H10K 50/11; C07D 307/79; C07D 413/10; H10K 50/15; H10K 50/16; H10K 85/60

(52) U.S. Cl. CPC H10K 50/11; C07D 307/79; C07D 413/10; H10K 50/15; H10K 50/16; H10K 85/622; H10K 85/626; H10K 85/633; H10K 85/636; H10K 85/654; H10K 85/6572; H10K 85/6574; H10K 85/6576

(57) ABSTRACT

The present specification relates to a heterocyclic compound of Chemical Formula 1, an organic light emitting device including the same and a composition for forming an organic material layer.

### TECHNICAL FIELD

This application claims priority to and the benefit of Korean Patent Application No. 10-2022-0040365 filed in the Korean Intellectual Property Office on Mar. 31, 2022, the entire contents of which are incorporated herein by reference.

The present specification relates to a heterocyclic compound, an organic light emitting device including the same and a composition for forming an organic material layer.

### BACKGROUND ART

An electroluminescence device is a kind of self-emitting type display device, and has an advantage in that the viewing angle is wide, the contrast is excellent, and the response speed is fast.

An organic light emitting device has a structure in which an organic thin film is disposed between two electrodes. When a voltage is applied to an organic light emitting device having the structure, electrons and holes injected from the two electrodes combine with each other in an organic thin film to make a pair, and then, emit light while being extinguished. The organic thin film may be composed of a single layer or multiple layers, if necessary.

A material for the organic thin film may have a light emitting function, if necessary. For example, as the material for the organic thin film, it is also possible to use a compound, which may itself constitute a light emitting layer alone, or it is also possible to use a compound, which may serve as a host or a dopant of a host-dopant-based light emitting layer. In addition, as a material for the organic thin film, it is also possible to use a compound, which may play a role such as a hole injection, hole transport, electron blocking, hole blocking, electron transport or electron injection.

In order to improve the performance, service life, or efficiency of the organic light emitting device, there is a continuous need for developing a material for an organic thin film.

### RELATED ART DOCUMENT

(Patent Document 1) U.S. Pat. No. 4,356,429

### DETAILED DESCRIPTION OF THE INVENTION

### Technical Problem

The present specification has been made in an effort to provide a heterocyclic compound, an organic light emitting device including the same and a composition for forming an organic material layer.

### Technical Solution

In an exemplary embodiment of the present specification, provided is a heterocyclic compound of the following Chemical Formula 1.

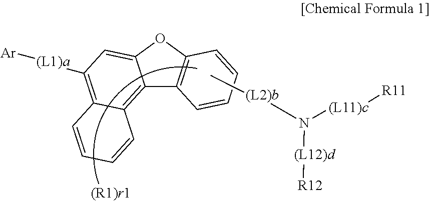

In Chemical Formula 1,
L1, L2, L11, and L12 are each independently a direct bond; or a substituted or unsubstituted C6 to C60 arylene group,
a, b, c, and d are an integer from 0 to 4,
when a, b, c, and d are 2 or higher, substituents in the parenthesis are the same as or different from each other,
Ar is a substituted or unsubstituted C6 to C60 aryl group; or a substituted or unsubstituted heteroaryl group having up to 3 rings containing O or S,
R1, R11, and R12 are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, and
r1 is an integer from 0 to 8, and when r1 is 2 or higher, R1's are the same as or different from each other.

In another exemplary embodiment of the present specification, provided is an organic light emitting device including: a first electrode; a second electrode; and an organic material layer having one or more layers provided between the first electrode and the second electrode, in which one or more layers of the organic material layer include one or more of the heterocyclic compounds.

In still another exemplary embodiment of the present specification, provided is a composition for forming an organic material layer, including the heterocyclic compound.

### Advantageous Effects

When used in an organic light emitting device, the heterocyclic compound described in the present specification can lower the driving voltage of the device, improve the light emitting efficiency, and improve the service life characteristics of the device. Specifically, the heterocyclic compound of the present invention is characterized in that it includes naphthobenzofuran as a core structure as shown in Chemical Formula 1, has one or more substituents on both benzene rings of the dibenzofuran structure in the core structure, in particular, has a substituent at position 3 based on the dibenzofuran structure, and needs to include an amine group at any one of positions 5 to 8.

As described above, since the heterocyclic compound according to the present application has a characteristic structure, the excellent hole transport properties of an amine functional group can adjust the band gap and T1 (triplet state energy level) values to adjust the hole transport ability and electron blocking ability, thereby lowering the driving voltage of the device and improving the light efficiency. Further, the heterocyclic compound of the present invention has an effect of improving the service life characteristics of the device by improving the thermal stability of the compound by bonding the other substituent (-(L1)a-Ar) of the two substituents to the specific position to strengthen hole characteristics and simultaneously increase the planarity and glass transition temperature of the amine derivative.

### BRIEF DESCRIPTION OF DRAWINGS

FIGS. 1 to 4 are views each exemplarily illustrating a stacking structure of an organic light emitting device according to an exemplary embodiment of the present specification.

### EXPLANATION OF REFERENCE NUMERALS AND SYMBOLS

100: Substrate
200: Positive electrode
300: Organic material layer
301: Hole injection layer
302: Hole transport layer
303: Electron blocking layer
304: Light Emitting Layer
305: Electron transport layer
306: Electron injection layer
400: Negative electrode

### Best Mode

Hereinafter, the present specification will be described in more detail.

When one part “includes” one constituent element in the present specification, unless otherwise specifically described, this does not mean that another constituent element is excluded, but means that another constituent element may be further included.

In the present specification,

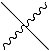

of a chemical formula means a position to which a constituent element is bonded.

The term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more are substituted, the two or more substituents may be the same as or different from each other.

In the present specification, “substituted or unsubstituted” means being unsubstituted or substituted with one or more substituents selected from the group consisting of deuterium; a halogen group; —CN; a C1 to C60 alkyl group; a C2 to C60 alkenyl group; a C2 to C60 alkynyl group; a C1 to C60 haloalkyl group; a C1 to C60 alkoxy group; a C6 to C60 aryloxy group; a C1 to C60 alkylthioxy group; a C6 to C60 arylthioxy group; a C1 to C60 alkylsulfoxy group; a C6 to C60 arylsulfoxy group; a C3 to C60 cycloalkyl group; a C2 to C60 heterocycloalkyl group; a C6 to C60 aryl group; a C2 to C60 heteroaryl group; —SiRR′R″; —P(═O)RR′; and —NRR′, or a substituent to which two or more substituents selected among the exemplified substituents are linked, and R, R′ and R″ are each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group.

In the present specification, “when a substituent is not indicated in the structure of a chemical formula or compound” means that a hydrogen atom is bonded to a carbon atom. However, since deuterium (²H) is an isotope of hydrogen, some hydrogen atoms may be deuterium.

In an exemplary embodiment of the present application, “when a substituent is not indicated in the structure of a chemical formula or compound” may mean that all the positions that may be reached by the substituent are hydrogen or deuterium. That is, deuterium is an isotope of hydrogen, and some hydrogen atoms may be deuterium which is an isotope, and in this case, the content of deuterium may be 0% to 100%.

In an exemplary embodiment of the present application, in “the case where a substituent is not indicated in the structure of a chemical formula or compound”, when the content of deuterium is 0%, the content of hydrogen is 100%, and all the substituents do not explicitly exclude deuterium such as hydrogen, hydrogen and deuterium may be mixed and used in the compound.

In an exemplary embodiment of the present application, deuterium is one of the isotopes of hydrogen, is an element that has a deuteron composed of one proton and one neutron as a nucleus, and may be represented by hydrogen-2, and the element symbol may also be expressed as D or ²H.

In an exemplary embodiment of the present application, the isotope means an atom with the same atomic number (Z), but different mass numbers (A), and may also be interpreted as an element which has the same number of protons, but different number of neutrons.

In an exemplary embodiment of the present application, when the total number of substituents of a basic compound is defined as T1 and the number of specific substituents among the substituents is defined as T2, the content T % of the specific substituent may be defined as T2/T1×100=T %.

That is, in an example, the deuterium content of 20% in a phenyl group represented by

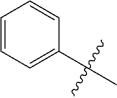 [SMILES: *c1ccccc1]

may be represented by 20% when the total number of substituents that the phenyl group can have is 5 (T1 in the formula) and the number of deuteriums among the substituents is 1 (T2 in the formula). That is, a deuterium content of 20% in the phenyl group may be represented by the following structural formula.

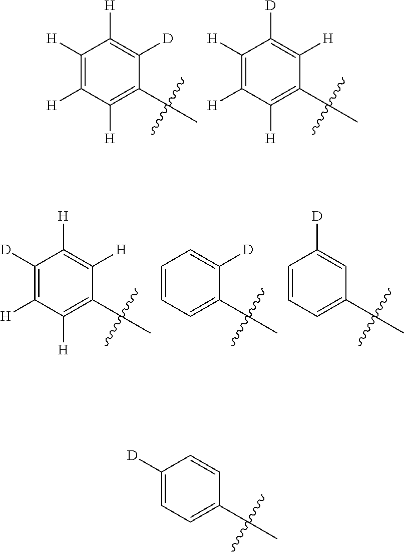

Further, in an exemplary embodiment of the present application, “a phenyl group having a deuterium content of 0%” may mean a phenyl group that does not include a deuterium atom, that is, has five hydrogen atoms.

In the present specification, the halogen may be fluorine, chlorine, bromine or iodine.

In the present specification, an alkyl group includes a straight-chain or branched-chain having 1 to 60 carbon atoms, and may be additionally substituted with another substituent. The number of carbon atoms of the alkyl group may be 1 to 60, specifically 1 to 40, and more specifically 1 to 20. Specific examples thereof include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like, but are not limited thereto.

In the present specification, an alkenyl group includes a straight-chain or branched-chain having 2 to 60 carbon atoms, and may be additionally substituted with another substituent. The number of carbon atoms of the alkenyl group may be 2 to 60, specifically 2 to 40, and more specifically 2 to 20. Specific examples thereof include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group, and the like, but are not limited thereto. In the present specification, an alkynyl group includes a straight-chain or branched-chain having 2 to 60 carbon atoms, and may be additionally substituted with another substituent. The number of carbon atoms of the alkynyl group may be 2 to 60, specifically 2 to 40, and more specifically 2 to 20.

In the present specification, a haloalkyl group means an alkyl group substituted with a halogen group, and specific examples thereof include —CF₃, —CF₂CF₃, and the like, but are not limited thereto.

In the present specification, an alkoxy group is represented by —O(R101), and the above-described examples of the alkyl group may be applied to R101.

In the present specification, an aryloxy group is represented by —O(R102), and the above-described examples of the aryl group may be applied to R102.

In the present specification, an alkylthioxy group is represented by —S(R103), and the above-described examples of the alkyl group may be applied to R103.

In the present specification, an arylthioxy group is represented by —S(R104), and the above-described examples of the aryl group may be applied to R104.

In the present specification, an alkylsulfoxy group is represented by —S(═O)₂(R105), and the above-described examples of the alkyl group may be applied to R105.

In the present specification, an arylsulfoxy group is represented by —S(═O)₂(R106), and the above-described examples of the aryl group may be applied to R106.

In the present specification, a cycloalkyl group includes a monocycle or polycycle having 3 to 60 carbon atoms, and may be additionally substituted with another substituent. Here, the polycycle means a group in which a cycloalkyl group is directly linked to or fused with another cyclic group. Here, another cyclic group may also be a cycloalkyl group, but may also be another kind of cyclic group, for example, a heterocycloalkyl group, an aryl group, a heteroaryl group, and the like. The number of carbon atoms of the cycloalkyl group may be 3 to 60, specifically 3 to 40, and more specifically 5 to 20. Specific examples thereof include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, and the like, but are not limited thereto.

In the present specification, a heterocycloalkyl group includes O, S, Se, N, or Si as a heteroatom, includes a monocycle or polycycle having 2 to 60 carbon atoms, and may be additionally substituted with another substituent. Here, the polycycle means a group in which a heterocycloalkyl group is directly linked to or fused with another cyclic group. Here, another cyclic group may also be a heterocycloalkyl group, but may also be another kind of cyclic group, for example, a cycloalkyl group, an aryl group, a heteroaryl group, and the like. The number of carbon atoms of the heterocycloalkyl group may be 2 to 60, specifically 2 to 40, and more specifically 3 to 20.

In the present specification, an aryl group includes a monocycle or polycycle having 6 to 60 carbon atoms, and may be additionally substituted with another substituent. Here, the polycycle means a group in which an aryl group is directly linked to or fused with another cyclic group. Here, another cyclic group may also be an aryl group, but may also be another kind of cyclic group, for example, a cycloalkyl group, a heterocycloalkyl group, a heteroaryl group, and the like. The aryl group includes a spiro group. The number of carbon atoms of the aryl group may be 6 to 60, specifically 6 to 40, and more specifically 6 to 25. Specific examples of the aryl group include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused cyclic group thereof, and the like, but are not limited thereto.

In the present specification, the terphenyl group may be selected from the following structures.

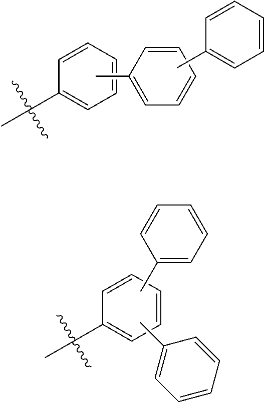

In the present specification, the fluorenyl group may be substituted, and adjacent substituents may be bonded to each other to form a ring.

When the fluorenyl group is substituted, the substituent may be the following structures, but is not limited thereto.

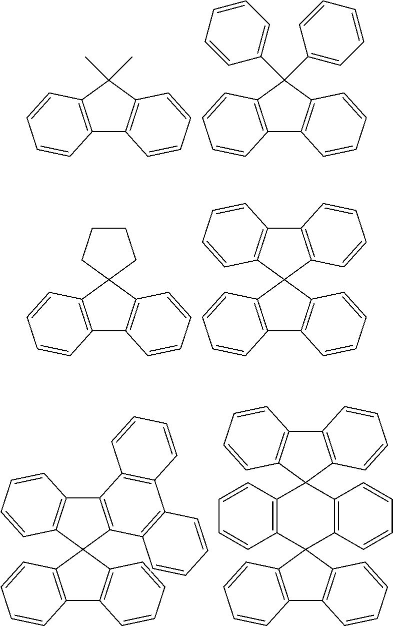

In the present specification, a heteroaryl group includes S, O, Se, N, or Si as a heteroatom, includes a monocycle or polycycle having 2 to 60 carbon atoms, and may be additionally substituted with another substituent. Here, the polycycle means a group in which a heteroaryl group is directly linked to or fused with another cyclic group. Here, another cyclic group may also be a heteroaryl group, but may also be another kind of cyclic group, for example, a cycloalkyl group, a heterocycloalkyl group, an aryl group, and the like. The number of carbon atoms of the heteroaryl group may be 2 to 60, specifically 2 to 40, and more specifically 3 to 25. Specific examples of the heteroaryl group include a pyridine group, a pyrrole group, a pyrimidine group, a pyridazine group, a furan group, a thiophene group, an imidazole group, a pyrazole group, an oxazole group, an isoxazole group, a thiazole group, an isothiazole group, a triazole group, a furazan group, an oxadiazole group, a thiadiazole group, a dithiazole group, a tetrazolyl group, a pyran group, a thiopyran group, a diazine group, an oxazine group, a thiazine group, a dioxin group, a triazine group, a tetrazine group, a quinoline group, an isoquinoline group, a quinazoline group, an isoquinazoline group, a quinozoline group, a naphthyridine group, an acridine group, a phenanthridine group, an imidazopyridine group, a diazanaphthalene group, a triazaindene group, an indole group, an indolizine group, a benzothiazole group, a benzoxazole group, a benzimidazole group, a benzothiophene group, a benzofuran group, a dibenzothiophene group, a dibenzofuran group, a carbazole group, a benzocarbazole group, a dibenzocarbazole group, a phenazine group, a dibenzosilole group, spirobi(dibenzosilole), a dihydrophenazine group, a phenoxazine group, a phenanthridine group, a thienyl group, an indolo[2,3-a]carbazole group, an indolo[2,3-b]carbazole group, an indoline group, a 10,11-dihydro-dibenzo[b,f]azepine group, a 9,10-dihydroacridine group, a phenanthrazine group, a phenothiazine group, a phthalazine group, a phenanthroline group, a naphthobenzofuran group, a naphthobenzothiophene group, a benzo[c][1,2,5]thiadiazole group, a 2,3-dihydrobenzo[b]thiophene group, a 2,3-dihydrobenzofuran group, a 5,10-dihydrodibenzo[b,e][1,4]azasiline group, a pyrazolo[1,5-c]quinazoline group, a pyrido[1,2-b]indazole group, a pyrido[1,2-a]imidazo[1,2-e]indoline group, a 5,11-dihydroindeno[1,2-b]carbazole group, and the like, but are not limited thereto.

In the present specification, a benzocarbazole group may be any one of the following structures.

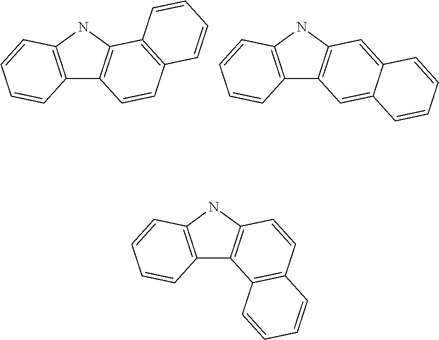

In the present specification, a dibenzocarbazole group may be any one of the following structures.

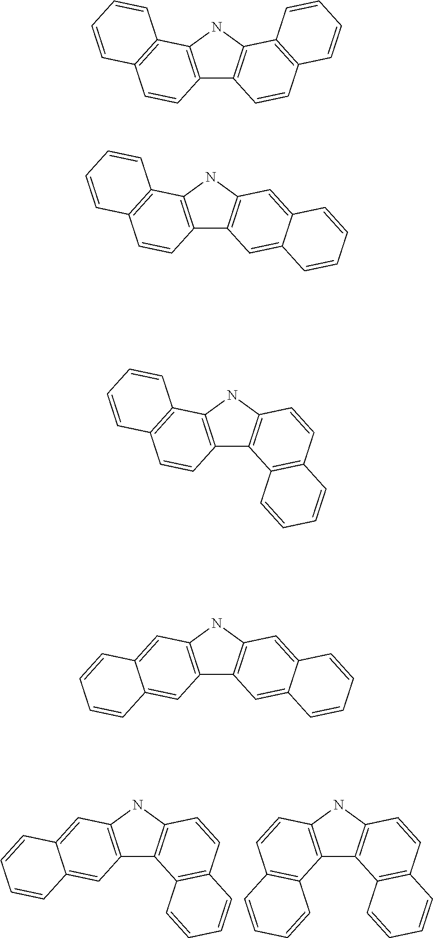

In the present specification, when the substituent is a carbazole group, a benzocarbazole group, or a dibenzocarbazole group, it means being bonded to the nitrogen or carbon of the carbazole group, the benzocarbazole group, or the dibenzocarbazole group.

In the present specification, when a carbazole group, a benzocarbazole group, or a dibenzocarbazole group is substituted, an additional substituent may be substituted at the nitrogen or carbon of the carbazole group, the benzocarbazole group, or the dibenzocarbazole group.

In the present specification, a naphthobenzofuran group may be any one of the following structures.

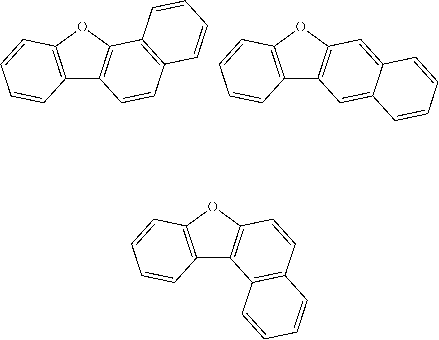

In the present specification, a naphthobenzothiophene group may be any one of the following structures.

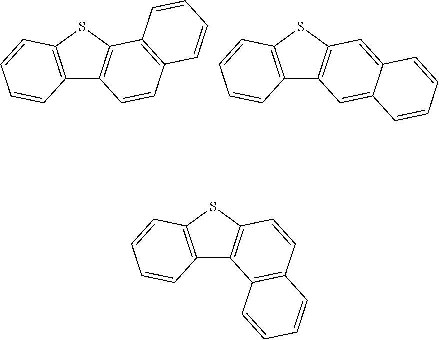

In the present specification, a silyl group includes Si and is a substituent to which the Si atom is directly linked as a radical, and is represented by —Si(R107) (R108) (R109), and R107 to R109 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specific examples of the silyl group include the following structures, but are not limited thereto.

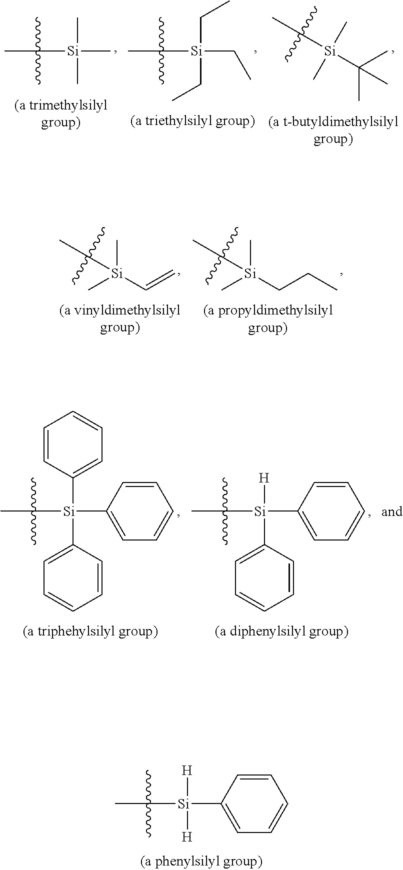

In the present specification, a phosphine oxide group is represented by —P(═O) (R110) (R111), and R110 and R111 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. Specifically, the phosphine oxide group may be substituted with an alkyl group or an aryl group, and the above-described example may be applied to the alkyl group and the aryl group. Examples of the phosphine oxide group include a dimethylphosphine oxide group, a diphenylphosphine oxide group, dinaphthylphosphine oxide, and the like, but are not limited thereto.

In the present specification, an amine group is represented by —N(R112) (R113), and R112 and R113 are the same as or different from each other, and may be each independently a substituent composed of at least one of hydrogen; deuterium; a halogen group; an alkyl group; an alkenyl group; an alkoxy group; a cycloalkyl group; a heterocycloalkyl group; an aryl group; and a heteroaryl group. The amine group may be selected from the group consisting of —NH₂; a monoalkylamine group; a monoarylamine group; a monoheteroarylamine group; a dialkylamine group; a diarylamine group; a diheteroarylamine group; an alkylarylamine group; an alkylheteroarylamine group; and an arylheteroarylamine group, and the number of carbon atoms thereof is not particularly limited, but is preferably 1 to 30. Specific examples of the amine group include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like, but are not limited thereto.

In the present specification, the above-described description of the aryl group may be applied to an arylene group except for a divalent arylene group.

In the present specification, the above-described description of the heteroaryl group may be applied to a heteroarylene group except for a divalent heteroarylene group.

An exemplary embodiment of the present specification provides the heterocyclic compound represented by Chemical Formula 1.

In an exemplary embodiment of the present specification, R1 is hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group.

In an exemplary embodiment of the present specification, R1 is hydrogen; deuterium; a halogen group; a cyano group; a substituted or unsubstituted C1 to C30 alkyl group; a substituted or unsubstituted C3 to C30 cycloalkyl group; a substituted or unsubstituted C2 to C30 heterocycloalkyl group; a substituted or unsubstituted C6 to C30 aryl group; or a substituted or unsubstituted C2 to C30 heteroaryl group.

In an exemplary embodiment of the present specification, R1 is hydrogen; deuterium; a substituted or unsubstituted C1 to C30 alkyl group; a substituted or unsubstituted C6 to C30 aryl group; or a substituted or unsubstituted C2 to C30 heteroaryl group.

In an exemplary embodiment of the present specification, R1 may be hydrogen; deuterium; a C1 to C30 alkyl group; a C6 to C30 aryl group; or a C2 to C30 heteroaryl group.

In an exemplary embodiment of the present specification, R1 may be hydrogen; or deuterium.

In an exemplary embodiment of the present specification, Chemical Formula 1 may be represented by any one of the following Chemical Formulae 1-1 to 1-4.

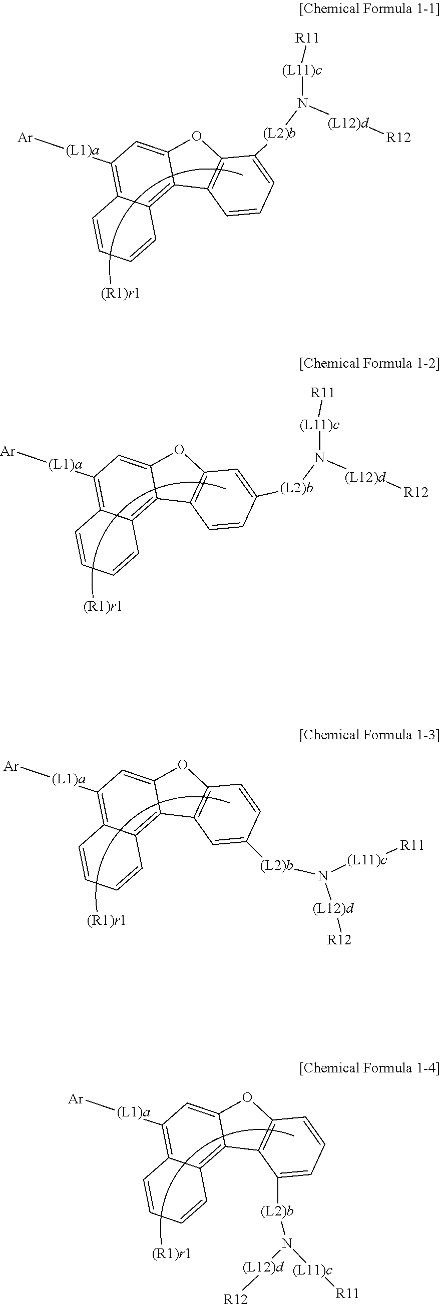

In Chemical Formulae 1-1 to 1-4,
the definition of each substituent is the same as that in Chemical Formula 1.

In an exemplary embodiment of the present specification, L1 and L2 are each independently a direct bond; or a substituted or unsubstituted C6 to C60 arylene group.

In an exemplary embodiment of the present specification, L1 and L2 may be each independently a direct bond; or a substituted or unsubstituted C6 to C30 arylene group.

In an exemplary embodiment of the present specification, L1 and L2 may be each independently a direct bond; a substituted or unsubstituted phenylene group; a substituted or unsubstituted biphenylene group; or a substituted or unsubstituted naphthylene group.

In an exemplary embodiment of the present specification, L1 and L2 may be each independently a direct bond; a phenylene group; a biphenylene group; or a naphthylene group.

In an exemplary embodiment of the present specification, L1 and L2 may be each independently a direct bond; or a C6 to C30 arylene group unsubstituted or substituted with deuterium.

In an exemplary embodiment of the present specification, L1 and L2 may be each independently a direct bond; a phenylene group unsubstituted or substituted with deuterium; a biphenylene group unsubstituted or substituted with deuterium; or a naphthylene group unsubstituted or substituted with deuterium.

In an exemplary embodiment of the present specification, Ar is a substituted or unsubstituted C6 to C60 aryl group; or a substituted or unsubstituted heteroaryl group having up to 3 rings containing O or S.

In an exemplary embodiment of the present specification, Ar may be a substituted or unsubstituted C6 to C30 aryl group; or a substituted or unsubstituted heteroaryl group having up to 3 rings containing O or S.

In an exemplary embodiment of the present specification, Ar may be a substituted or unsubstituted phenyl group; a substituted or unsubstituted biphenyl group; a substituted or unsubstituted terphenyl group; a substituted or unsubstituted naphthyl group; a substituted or unsubstituted dibenzofuran group; or a substituted or unsubstituted dibenzothiophene group.

In an exemplary embodiment of the present specification, Ar may be a C6 to C30 aryl group unsubstituted or substituted with deuterium; or a heteroaryl group unsubstituted or substituted with deuterium and having up to 3 rings containing O or S.

In an exemplary embodiment of the present specification, Ar may be a phenyl group unsubstituted or substituted with deuterium; a biphenyl group unsubstituted or substituted with deuterium; a terphenyl group unsubstituted or substituted with deuterium; a naphthyl group unsubstituted or substituted with deuterium; a dibenzofuran group unsubstituted or substituted with deuterium; or a dibenzothiophene group unsubstituted or substituted with deuterium.

In an exemplary embodiment of the present specification, L11 and L12 are each independently a direct bond; or a substituted or unsubstituted C6 to C60 arylene group.

In an exemplary embodiment of the present specification, L11 and L12 may be each independently a direct bond; or a substituted or unsubstituted C6 to C30 arylene group.

In an exemplary embodiment of the present specification, L11 and L12 may be each independently a direct bond; a substituted or unsubstituted phenylene group; a substituted or unsubstituted biphenylene group; or a substituted or unsubstituted naphthylene group.

In an exemplary embodiment of the present specification, L11 and L12 may be each independently a direct bond; a phenylene group; a biphenylene group; or a naphthylene group.

In an exemplary embodiment of the present specification, L11 and L12 may be each independently a direct bond; or a substituted or unsubstituted phenylene group.

In an exemplary embodiment of the present specification, L11 and L12 may be each independently a direct bond; or a phenylene group.

In an exemplary embodiment of the present specification, L11 and L12 may be each independently a direct bond; or a C6 to C30 arylene group unsubstituted or substituted with deuterium.

In an exemplary embodiment of the present specification, L11 and L12 may be each independently a direct bond; a phenylene group unsubstituted or substituted with deuterium; a biphenylene group unsubstituted or substituted with deuterium; or a naphthylene group unsubstituted or substituted with deuterium.

In an exemplary embodiment of the present specification, L11 and L12 may be each independently a direct bond; or a phenylene group unsubstituted or substituted with deuterium.

In an exemplary embodiment of the present specification, R11 and R12 may be each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group.

In an exemplary embodiment of the present specification, R11 and R12 are each independently a substituted or unsubstituted C1 to C40 alkyl group; a substituted or unsubstituted C3 to C40 cycloalkyl group; a substituted or unsubstituted C2 to C40 heterocycloalkyl group; a substituted or unsubstituted C6 to C40 aryl group; or a substituted or unsubstituted C2 to C40 heteroaryl group.

In an exemplary embodiment of the present specification, R11 and R12 are each independently a substituted or unsubstituted C6 to C40 aryl group; or a substituted or unsubstituted C2 to C40 heteroaryl group.

In an exemplary embodiment of the present specification, R11 and R12 are each independently a substituted or unsubstituted C6 to C40 aryl group; or a substituted or unsubstituted heteroaryl group having up to 3 rings containing O or S.

In an exemplary embodiment of the present specification, R11 and R12 may be each independently a substituted or unsubstituted C6 to C30 aryl group; or a substituted or unsubstituted heteroaryl group having up to 3 rings containing O or S.

In an exemplary embodiment of the present specification, R11 and R12 may be each independently a substituted or unsubstituted phenyl group; a substituted or unsubstituted biphenyl group; a substituted or unsubstituted terphenyl group; a substituted or unsubstituted naphthyl group; a substituted or unsubstituted fluorenyl group; a substituted or unsubstituted phenanthrene group; a substituted or unsubstituted triphenylene group; a substituted or unsubstituted dibenzofuran group; or a substituted or unsubstituted dibenzothiophene group.

In an exemplary embodiment of the present specification, R11 and R12 may be each independently C6 to C30 aryl group unsubstituted or substituted with deuterium; or a heteroaryl group unsubstituted or substituted with deuterium and having up to 3 rings containing O or S.

In an exemplary embodiment of the present specification, R11 and R12 may be each independently a phenyl group unsubstituted or substituted with deuterium; a biphenyl group unsubstituted or substituted with deuterium; a terphenyl group unsubstituted or substituted with deuterium; a naphthyl group unsubstituted or substituted with deuterium; a fluorenyl group unsubstituted or substituted with one or more substituents of deuterium, an alkyl group, and an aryl group; a phenanthrene group unsubstituted or substituted with deuterium; a triphenylene group unsubstituted or substituted with deuterium; a dibenzofuran group unsubstituted or substituted with deuterium; or a dibenzothiophene group unsubstituted or substituted with deuterium.

In an exemplary embodiment of the present application, provided is a heterocyclic compound wherein a deuterium content of Chemical Formula 1 is 0%, or 10% to 100%.

In an exemplary embodiment of the present specification, the deuterium content of Chemical Formula 1 may be 0% to 100%.

In an exemplary embodiment of the present specification, the deuterium content of Chemical Formula 1 may be 0%.

In an exemplary embodiment of the present specification, the deuterium content of Chemical Formula 1 may be 30% to 100%.

In an exemplary embodiment of the present specification, the deuterium content of Chemical Formula 1 may be 50% to 100%.

In an exemplary embodiment of the present specification, the deuterium content of Chemical Formula 1 may be 70% to 100%.

In an exemplary embodiment of the present specification, the deuterium content of Chemical Formula 1 may be 100%.

In an exemplary embodiment of the present specification, the deuterium content of the heterocyclic compound of Chemical Formula 1 satisfies the above range, the photochemical characteristics of a compound which includes deuterium and a compound which does not include deuterium are almost similar, but when deposited on a thin film, the deuterium-containing material tends to be packed with a narrower intermolecular distance.

Accordingly, when an electron only device (EOD) and a hole only device (HOD) are manufactured and the current density thereof according to voltage is confirmed, it can be confirmed that among the heterocyclic compounds of Chemical Formula 1 of the present invention, a compound including deuterium exhibits much more balanced charge transport characteristics than a compound which does not include deuterium.

Further, when the surface of a thin film is observed using an atomic force microscope (AFM), it can be confirmed that the thin film made of a compound including deuterium is deposited with a more uniform surface without any aggregated portion.

Additionally, since the single bond dissociation energy of carbon and deuterium is higher than the single bond dissociation energy of carbon and hydrogen, among the heterocyclic compounds of Chemical Formula 1 of the present invention, a compound in which the deuterium content satisfies the above range has the increased stability of the total molecules, so that there is an effect that the service life of the device is improved.

In an exemplary embodiment of the present specification, the deuterium content of Chemical Formula 1 may be 0% or 10% to 100%.

In an exemplary embodiment of the present specification, the deuterium content of Chemical Formula 1 may be 0% or 15% to 100%.

In an exemplary embodiment of the present specification, the deuterium content of Chemical Formula 1 may be 0% or 20% to 100%.

In an exemplary embodiment of the present specification, the deuterium content of Chemical Formula 1 may be 0% or 50% to 100%.

In an exemplary embodiment of the present specification, the deuterium content of Chemical Formula 1 may be 0% or 100%.

In an exemplary embodiment of the present specification, Chemical Formula 1 may be represented by any one of the following compounds.

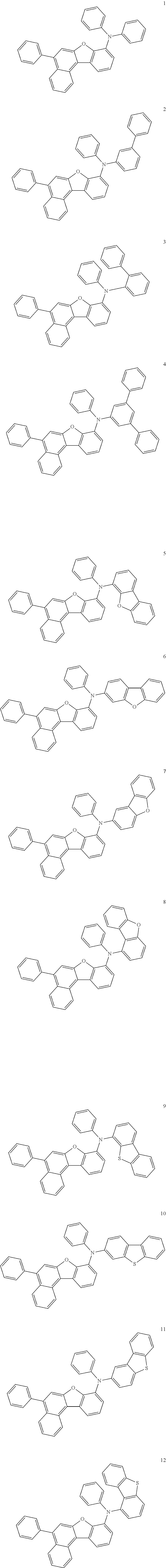

[chemical structure]

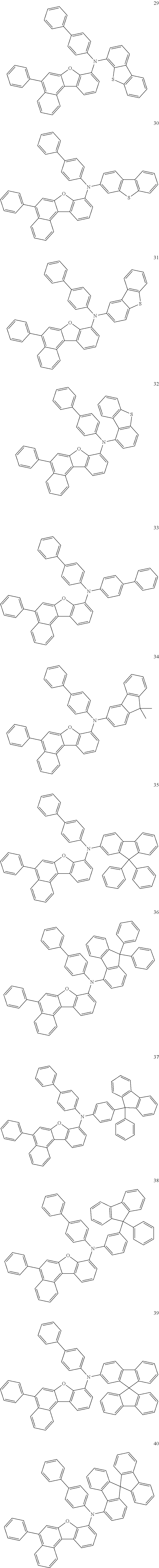

[chemical structure]

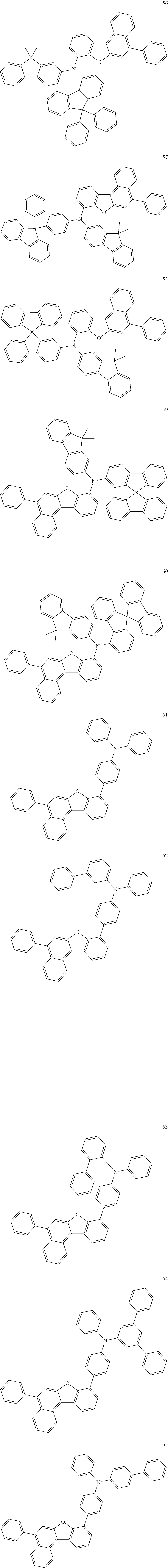

[chemical structure]

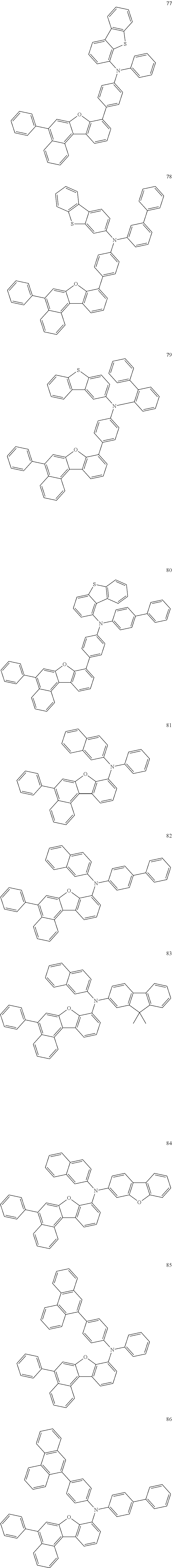

[chemical structure]

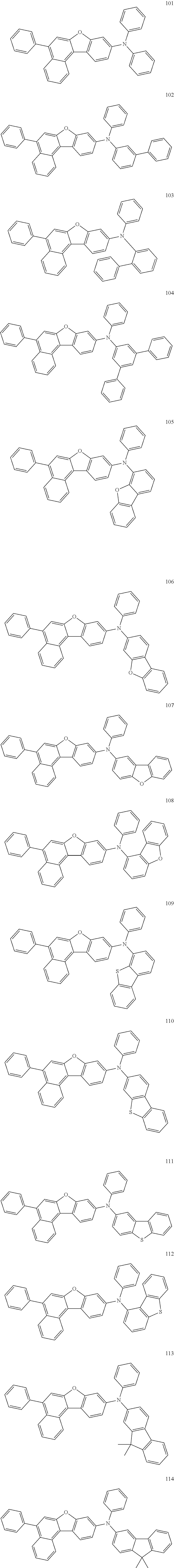

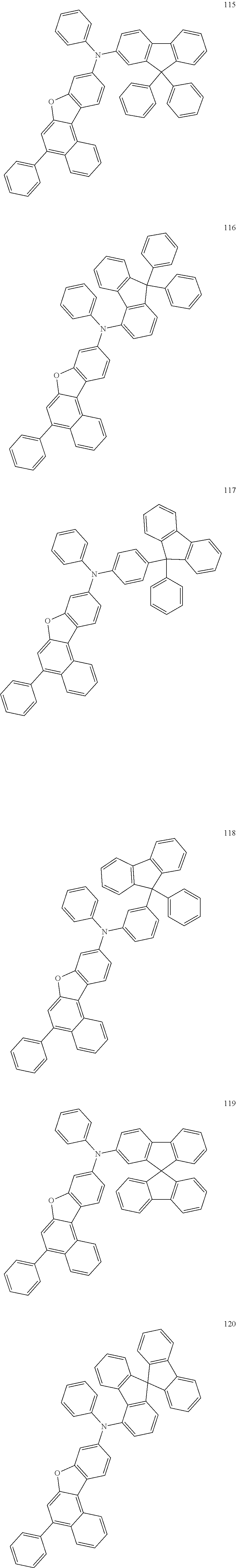

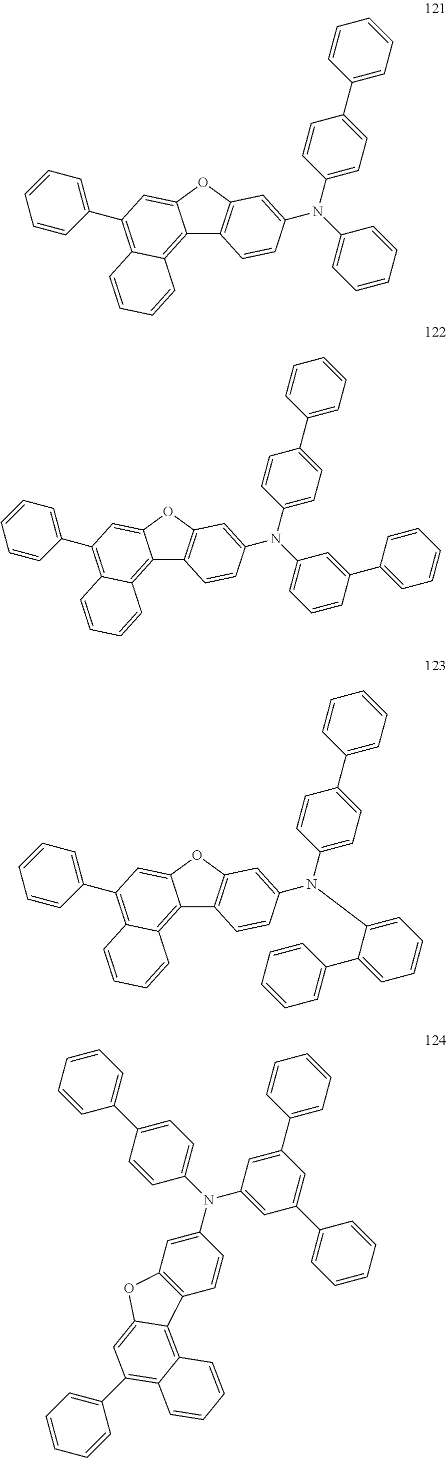 [SMILES: c1ccc(-c2ccc(N(c3ccccc3)c3ccc4c(c3)oc3cc(-c5ccccc5)c5ccccc5c34)cc2)cc1 ; c1ccc(-c2ccc(N(c3cccc(-c4ccccc4)c3)c3ccc4c(c3)oc3cc(-c5ccccc5)c5ccccc5c34)cc2)cc1 ; c1ccc(-c2ccc(N(c3ccc4c(c3)oc3cc(-c5ccccc5)c5ccccc5c34)c3ccccc3-c3ccccc3)cc2)cc1 ; c1ccc(-c2ccc(N(c3cc(-c4ccccc4)cc(-c4ccccc4)c3)c3ccc4c(c3)oc3cc(-c5ccccc5)c5ccccc5c34)cc2)cc1]

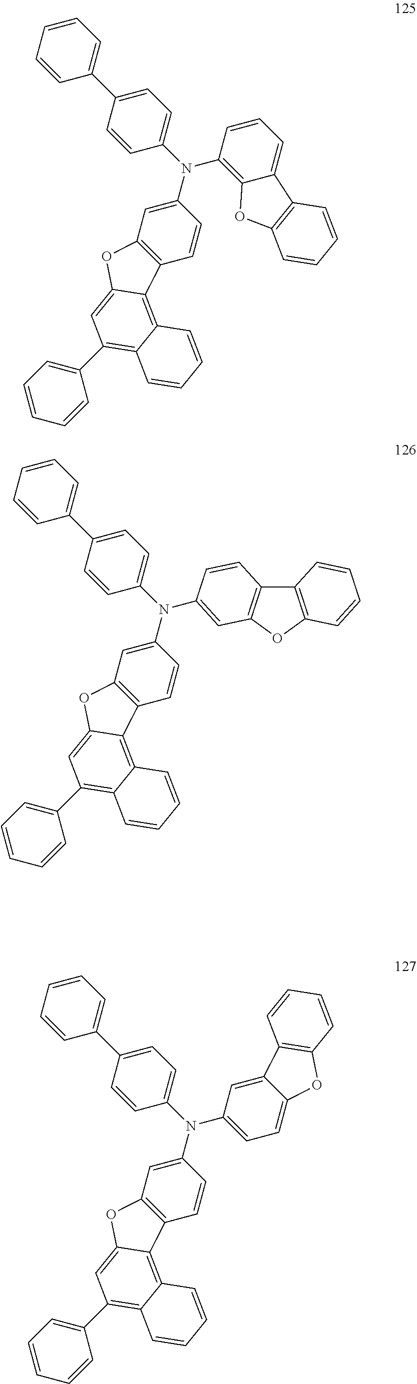

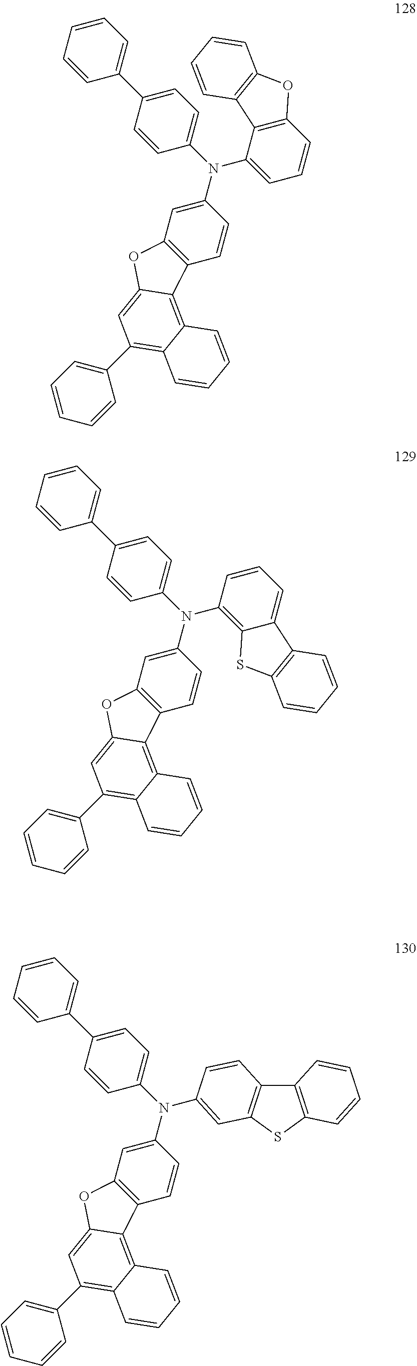

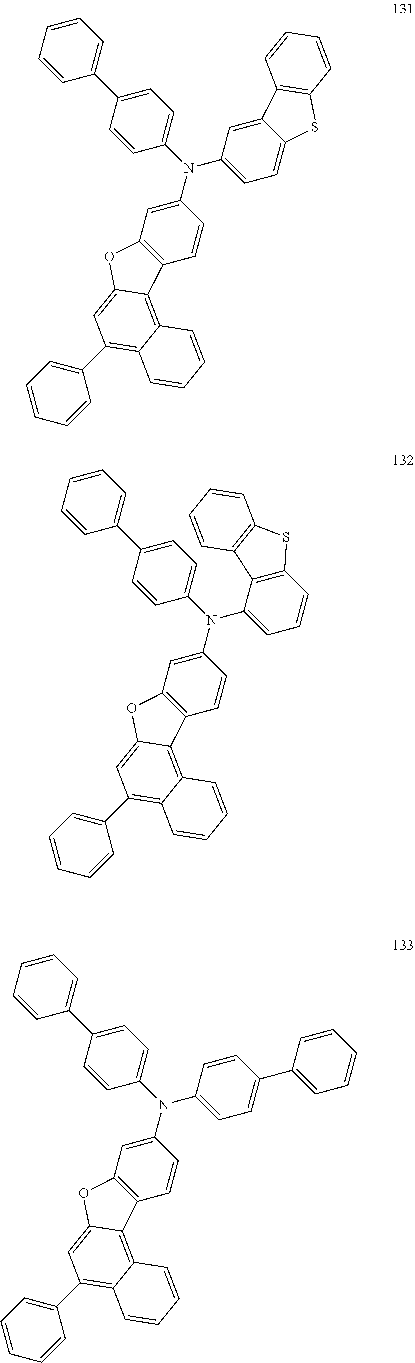 [SMILES: c1ccc(-c2ccc(N(c3ccc4c(c3)oc3cc(-c5ccccc5)c5ccccc5c34)c3ccc4sc5ccccc5c4c3)cc2)cc1 ; c1ccc(-c2ccc(N(c3ccc4c(c3)oc3cc(-c5ccccc5)c5ccccc5c34)c3cccc4sc5ccccc5c34)cc2)cc1 ; c1ccc(-c2ccc(N(c3ccc(-c4ccccc4)cc3)c3ccc4c(c3)oc3cc(-c5ccccc5)c5ccccc5c34)cc2)cc1]

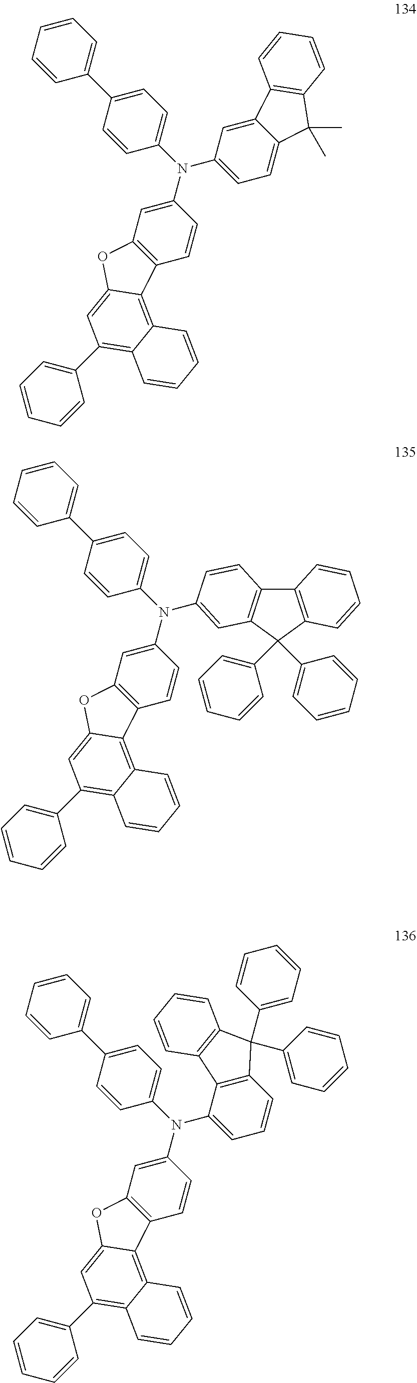

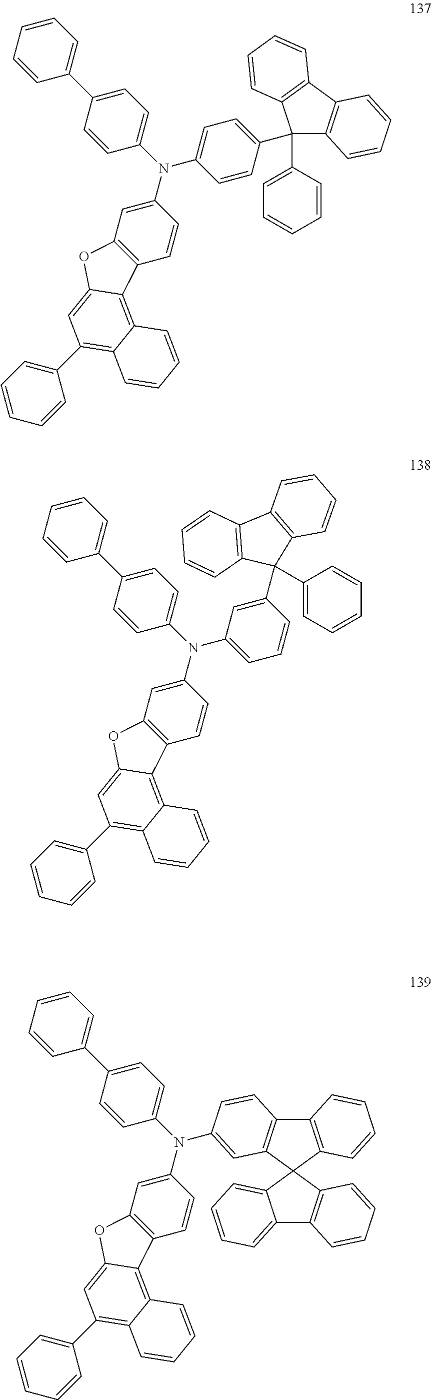 [SMILES: c1ccc(-c2ccc(N(c3ccc(C4(c5ccccc5)c5ccccc5-c5ccccc54)cc3)c3ccc4c(c3)oc3cc(-c5ccccc5)c5ccccc5c34)cc2)cc1 ; c1ccc(-c2ccc(N(c3cccc(C4(c5ccccc5)c5ccccc5-c5ccccc54)c3)c3ccc4c(c3)oc3cc(-c5ccccc5)c5ccccc5c34)cc2)cc1 ; c1ccc(-c2ccc(N(c3ccc4c(c3)C3(c5ccccc5-c5ccccc53)c3ccccc3-4)c3ccc4c(c3)oc3cc(-c5ccccc5)c5ccccc5c34)cc2)cc1]

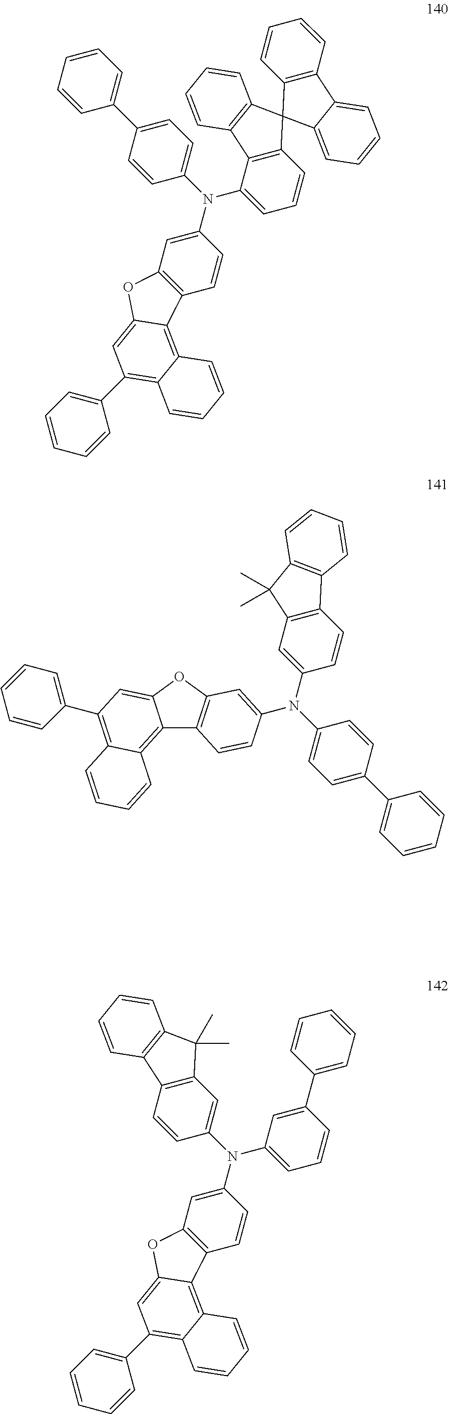 [SMILES: c1ccc(-c2ccc(N(c3ccc4c(c3)oc3cc(-c5ccccc5)c5ccccc5c34)c3cccc4c3-c3ccccc3C43c4ccccc4-c4ccccc43)cc2)cc1 ; CC1(C)c2ccccc2-c2ccc(N(c3ccc(-c4ccccc4)cc3)c3ccc4c(c3)oc3cc(-c5ccccc5)c5ccccc5c34)cc21 ; CC1(C)c2ccccc2-c2ccc(N(c3cccc(-c4ccccc4)c3)c3ccc4c(c3)oc3cc(-c5ccccc5)c5ccccc5c34)cc21]

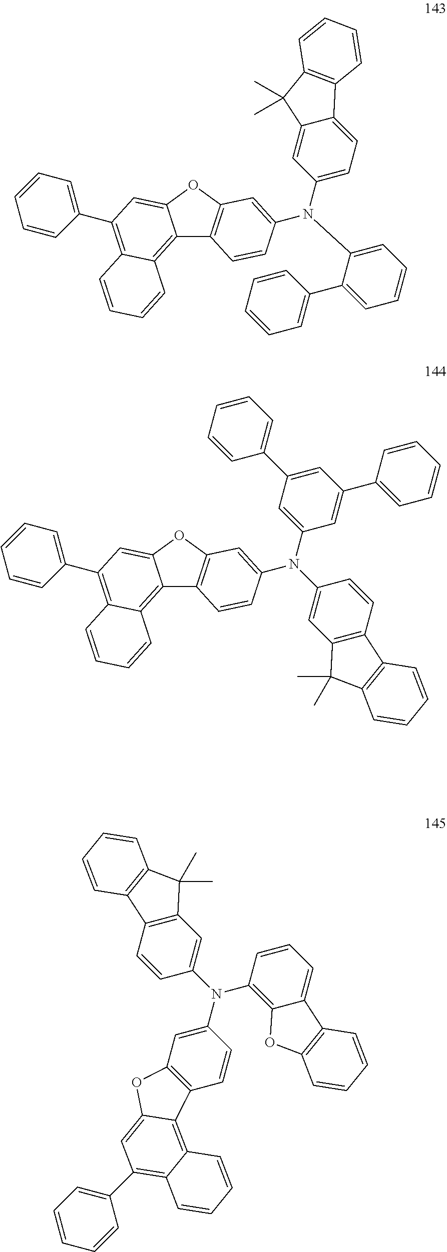 [SMILES: CC1(C)c2ccccc2-c2ccc(N(c3ccc4c(c3)oc3cc(-c5ccccc5)c5ccccc5c34)c3ccccc3-c3ccccc3)cc21 ; CC1(C)c2ccccc2-c2ccc(N(c3cc(-c4ccccc4)cc(-c4ccccc4)c3)c3ccc4c(c3)oc3cc(-c5ccccc5)c5ccccc5c34)cc21 ; CC1(C)c2ccccc2-c2ccc(N(c3ccc4c(c3)oc3cc(-c5ccccc5)c5ccccc5c34)c3cccc4c3oc3ccccc34)cc21]

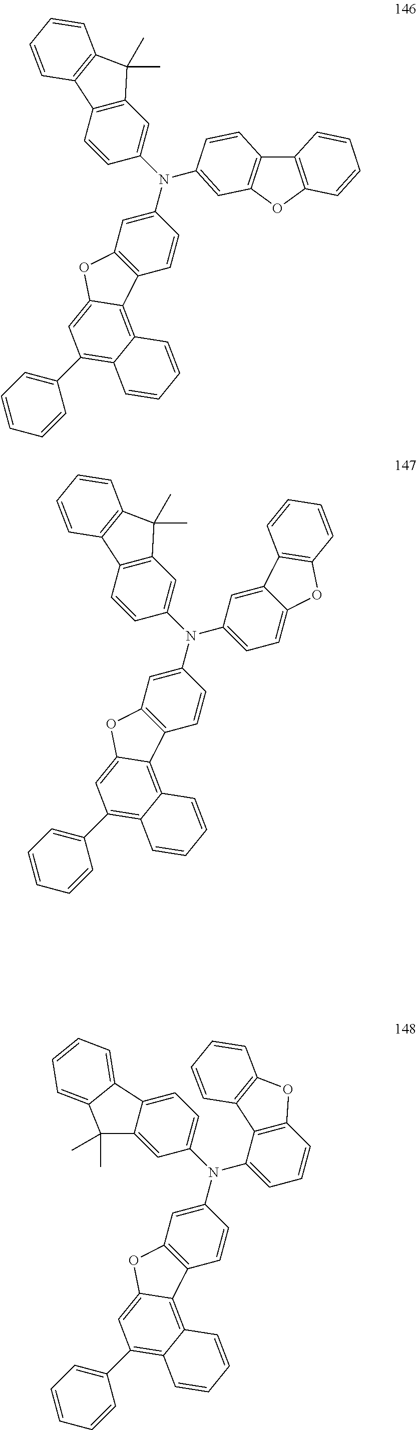 [SMILES: CC1(C)c2ccccc2-c2ccc(N(c3ccc4c(c3)oc3ccccc34)c3ccc4c(c3)oc3cc(-c5ccccc5)c5ccccc5c34)cc21 ; CC1(C)c2ccccc2-c2ccc(N(c3ccc4c(c3)oc3cc(-c5ccccc5)c5ccccc5c34)c3ccc4oc5ccccc5c4c3)cc21 ; CC1(C)c2ccccc2-c2ccc(N(c3ccc4c(c3)oc3cc(-c5ccccc5)c5ccccc5c34)c3cccc4oc5ccccc5c34)cc21]

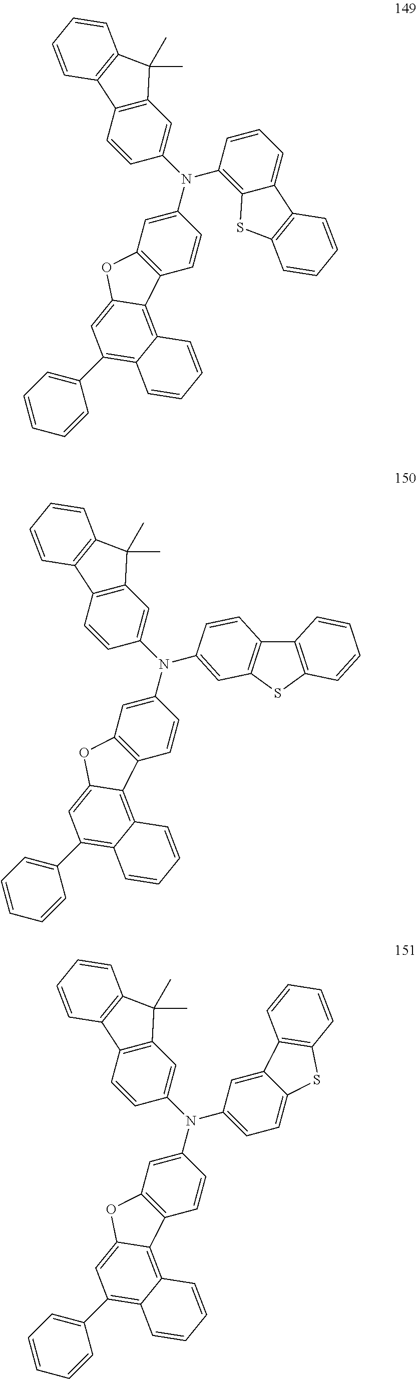 [SMILES: CC1(C)c2ccccc2-c2ccc(N(c3ccc4c(c3)oc3cc(-c5ccccc5)c5ccccc5c34)c3cccc4c3sc3ccccc34)cc21 ; CC1(C)c2ccccc2-c2ccc(N(c3ccc4c(c3)oc3cc(-c5ccccc5)c5ccccc5c34)c3ccc4c(c3)sc3ccccc34)cc21 ; CC1(C)c2ccccc2-c2ccc(N(c3ccc4c(c3)oc3cc(-c5ccccc5)c5ccccc5c34)c3ccc4sc5ccccc5c4c3)cc21]

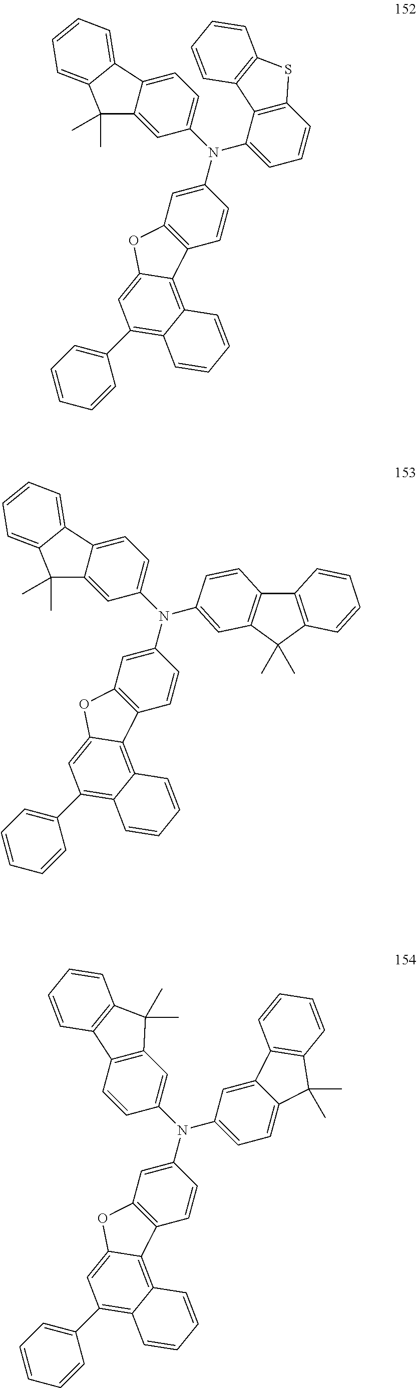 [SMILES: CC1(C)c2ccccc2-c2ccc(N(c3ccc4c(c3)oc3cc(-c5ccccc5)c5ccccc5c34)c3cccc4sc5ccccc5c34)cc21 ; CC1(C)c2ccccc2-c2ccc(N(c3ccc4c(c3)C(C)(C)c3ccccc3-4)c3ccc4c(c3)oc3cc(-c5ccccc5)c5ccccc5c34)cc21 ; CC1(C)c2ccccc2-c2cc(N(c3ccc4c(c3)C(C)(C)c3ccccc3-4)c3ccc4c(c3)oc3cc(-c5ccccc5)c5ccccc5c34)ccc21]

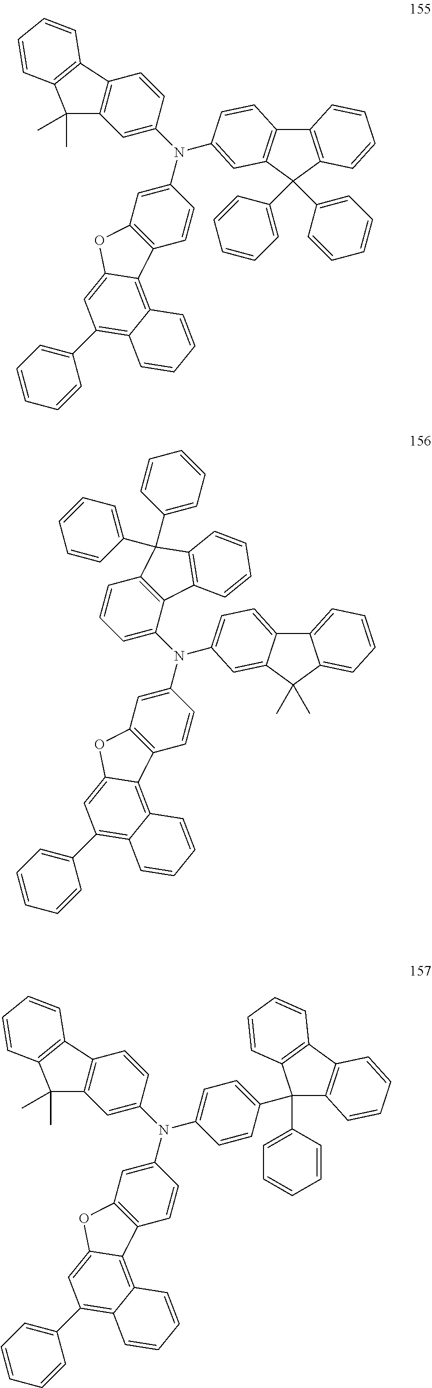 [SMILES: CC1(C)c2ccccc2-c2ccc(N(c3ccc4c(c3)C(c3ccccc3)(c3ccccc3)c3ccccc3-4)c3ccc4c(c3)oc3cc(-c5ccccc5)c5ccccc5c34)cc21 ; CC1(C)c2ccccc2-c2ccc(N(c3ccc4c(c3)oc3cc(-c5ccccc5)c5ccccc5c34)c3cccc4c3-c3ccccc3C4(c3ccccc3)c3ccccc3)cc21 ; CC1(C)c2ccccc2-c2ccc(N(c3ccc(C4(c5ccccc5)c5ccccc5-c5ccccc54)cc3)c3ccc4c(c3)oc3cc(-c5ccccc5)c5ccccc5c34)cc21]

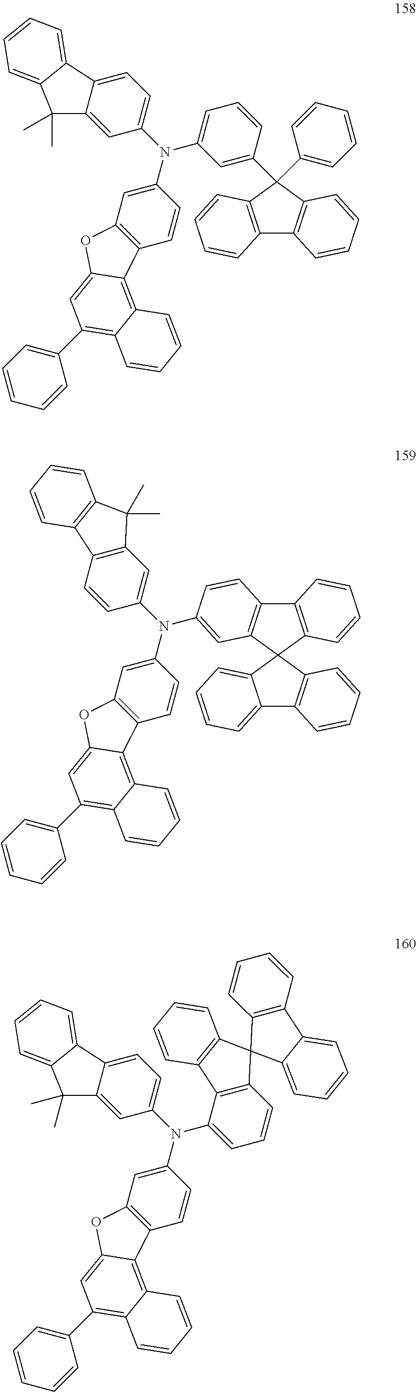 [SMILES: CC1(C)c2ccccc2-c2ccc(N(c3cccc(C4(c5ccccc5)c5ccccc5-c5ccccc54)c3)c3ccc4c(c3)oc3cc(-c5ccccc5)c5ccccc5c34)cc21 ; CC1(C)c2ccccc2-c2ccc(N(c3ccc4c(c3)C3(c5ccccc5-c5ccccc53)c3ccccc3-4)c3ccc4c(c3)oc3cc(-c5ccccc5)c5ccccc5c34)cc21 ; CC1(C)c2ccccc2-c2ccc(N(c3ccc4c(c3)oc3cc(-c5ccccc5)c5ccccc5c34)c3cccc4c3-c3ccccc3C43c4ccccc4-c4ccccc43)cc21]

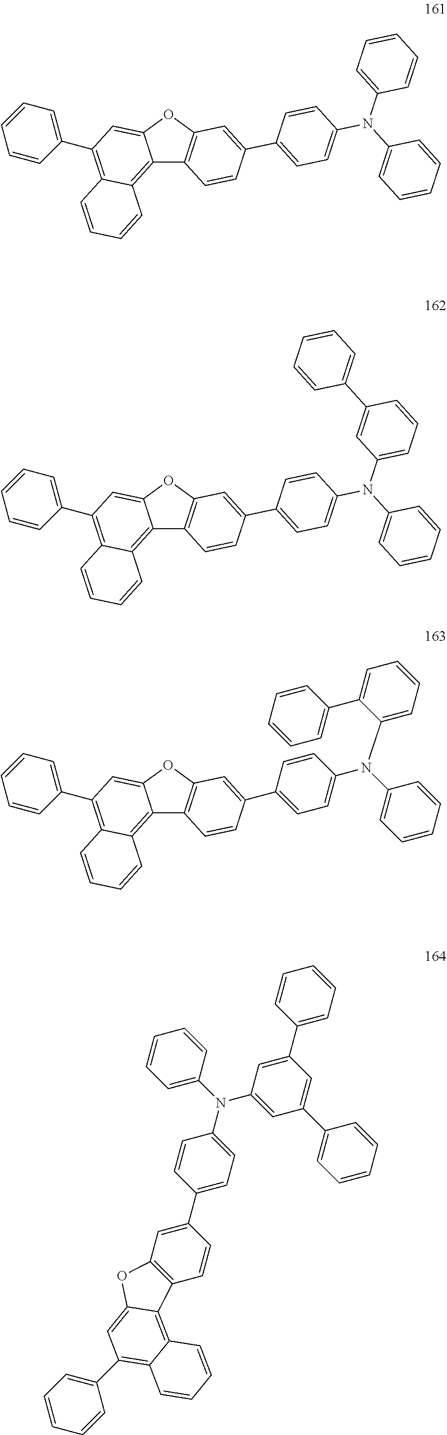 [SMILES: c1ccc(-c2cc3oc4cc(-c5ccc(N(c6ccccc6)c6ccccc6)cc5)ccc4c3c3ccccc23)cc1 ; c1ccc(-c2cccc(N(c3ccccc3)c3ccc(-c4ccc5c(c4)oc4cc(-c6ccccc6)c6ccccc6c45)cc3)c2)cc1 ; c1ccc(-c2ccccc2N(c2ccccc2)c2ccc(-c3ccc4c(c3)oc3cc(-c5ccccc5)c5ccccc5c34)cc2)cc1 ; c1ccc(-c2cc(-c3ccccc3)cc(N(c3ccccc3)c3ccc(-c4ccc5c(c4)oc4cc(-c6ccccc6)c6ccccc6c45)cc3)c2)cc1]

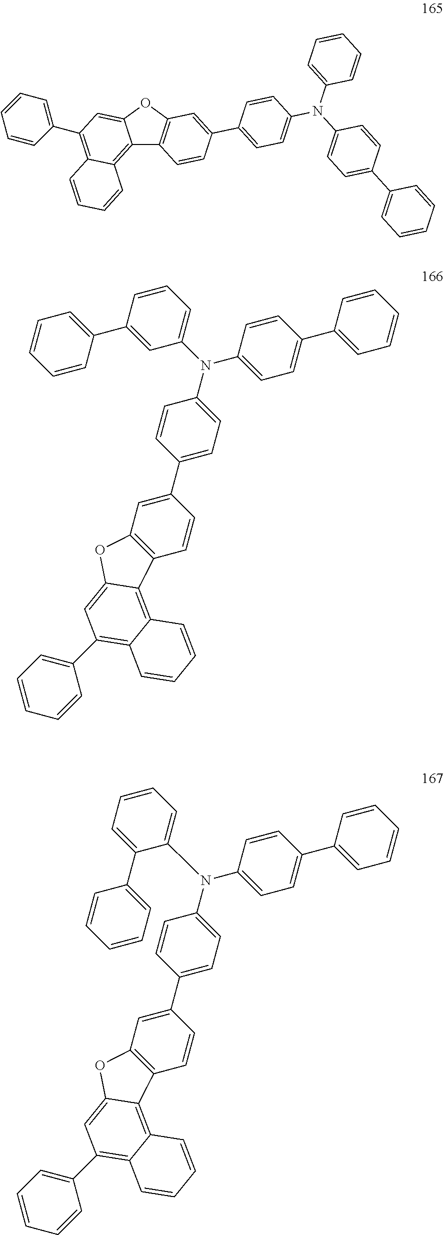 [SMILES: c1ccc(-c2ccc(N(c3ccccc3)c3ccc(-c4ccc5c(c4)oc4cc(-c6ccccc6)c6ccccc6c45)cc3)cc2)cc1 ; c1ccc(-c2ccc(N(c3ccc(-c4ccc5c(c4)oc4cc(-c6ccccc6)c6ccccc6c45)cc3)c3cccc(-c4ccccc4)c3)cc2)cc1 ; c1ccc(-c2ccc(N(c3ccc(-c4ccc5c(c4)oc4cc(-c6ccccc6)c6ccccc6c45)cc3)c3ccccc3-c3ccccc3)cc2)cc1]

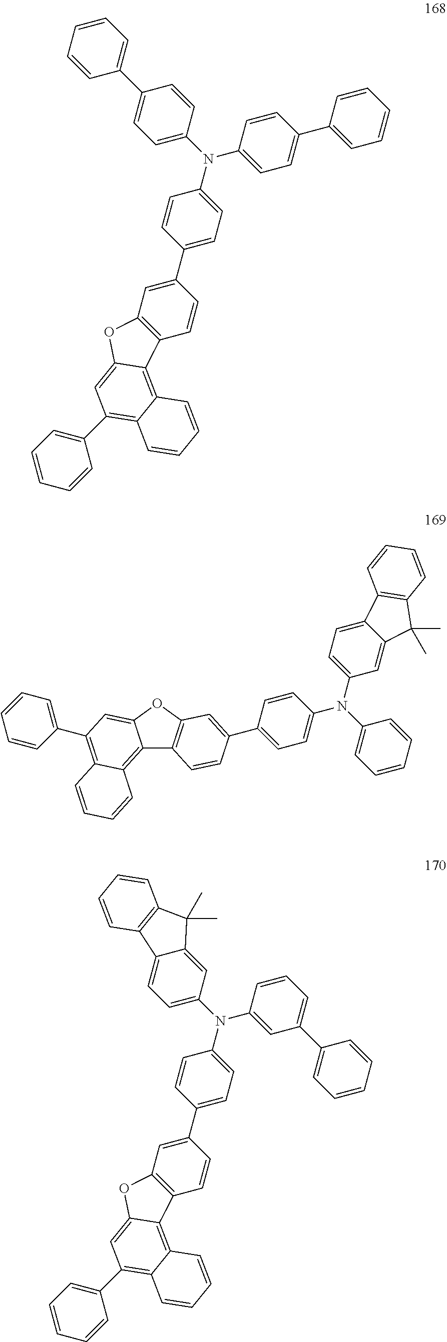 [SMILES: c1ccc(-c2ccc(N(c3ccc(-c4ccccc4)cc3)c3ccc(-c4ccc5c(c4)oc4cc(-c6ccccc6)c6ccccc6c45)cc3)cc2)cc1 ; CC1(C)c2ccccc2-c2ccc(N(c3ccccc3)c3ccc(-c4ccc5c(c4)oc4cc(-c6ccccc6)c6ccccc6c45)cc3)cc21 ; CC1(C)c2ccccc2-c2ccc(N(c3ccc(-c4ccc5c(c4)oc4cc(-c6ccccc6)c6ccccc6c45)cc3)c3cccc(-c4ccccc4)c3)cc21]

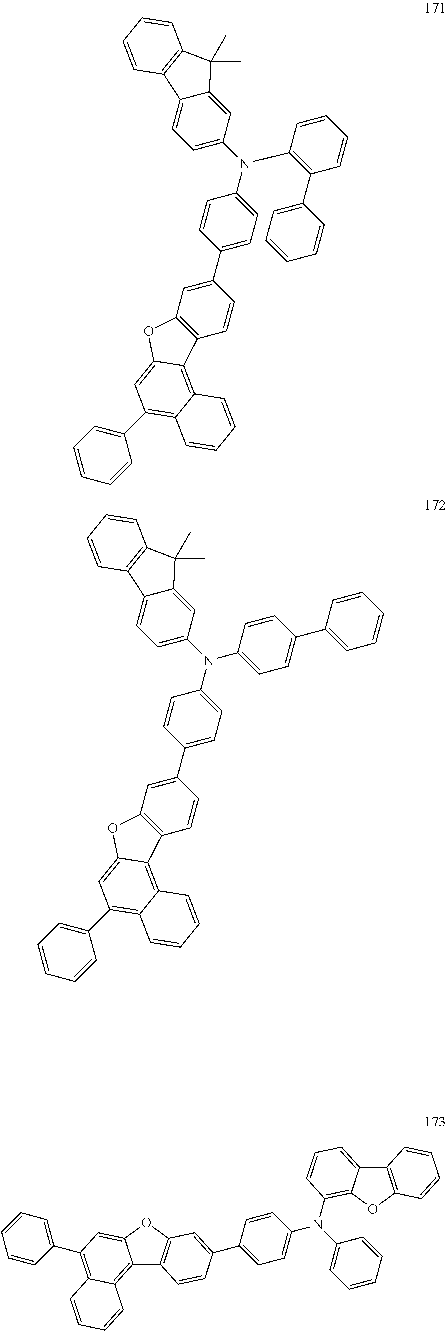

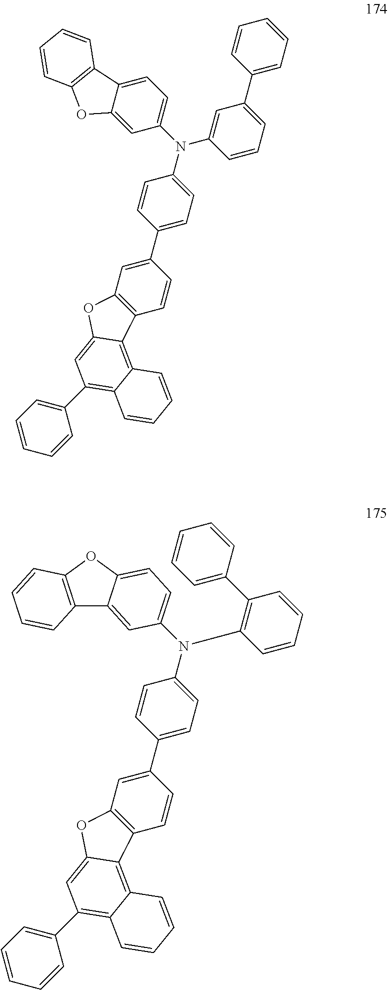 [SMILES: c1ccc(-c2cccc(N(c3ccc(-c4ccc5c(c4)oc4cc(-c6ccccc6)c6ccccc6c45)cc3)c3ccc4c(c3)oc3ccccc34)c2)cc1 ; c1ccc(-c2ccccc2N(c2ccc(-c3ccc4c(c3)oc3cc(-c5ccccc5)c5ccccc5c34)cc2)c2ccc3oc4ccccc4c3c2)cc1]

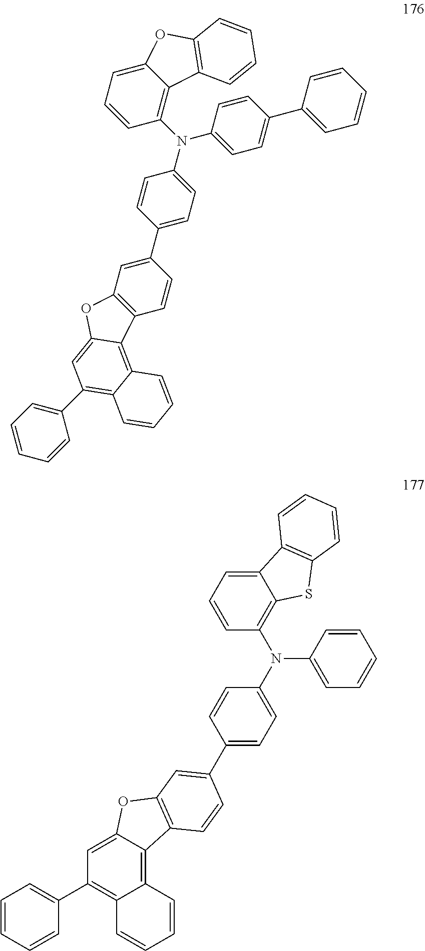 [SMILES: c1ccc(-c2ccc(N(c3ccc(-c4ccc5c(c4)oc4cc(-c6ccccc6)c6ccccc6c45)cc3)c3cccc4oc5ccccc5c34)cc2)cc1 ; c1ccc(-c2cc3oc4cc(-c5ccc(N(c6ccccc6)c6cccc7c6sc6ccccc67)cc5)ccc4c3c3ccccc23)cc1]

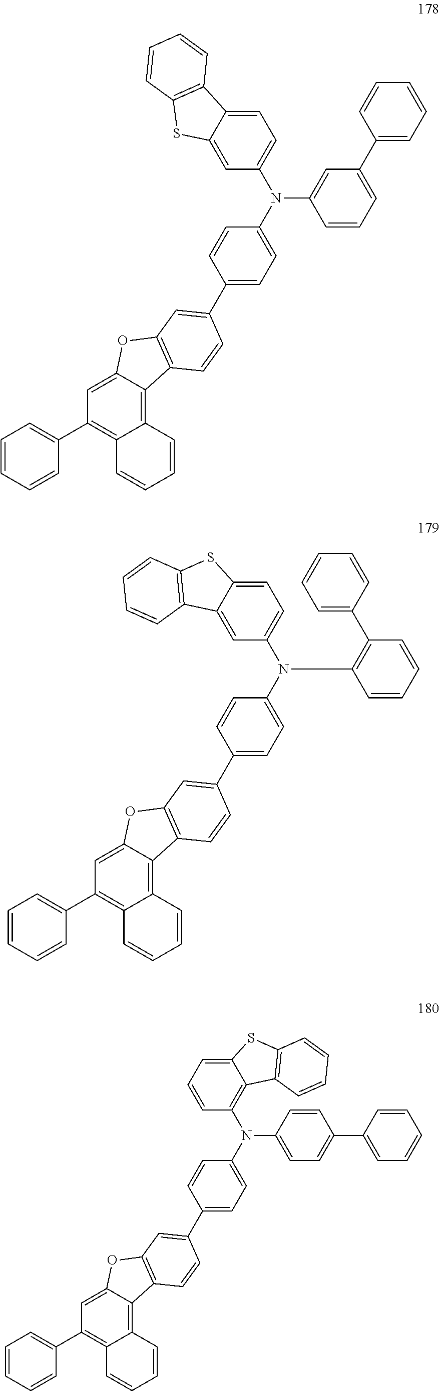 [SMILES: c1ccc(-c2cccc(N(c3ccc(-c4ccc5c(c4)oc4cc(-c6ccccc6)c6ccccc6c45)cc3)c3ccc4c(c3)sc3ccccc34)c2)cc1 ; c1ccc(-c2ccccc2N(c2ccc(-c3ccc4c(c3)oc3cc(-c5ccccc5)c5ccccc5c34)cc2)c2ccc3sc4ccccc4c3c2)cc1 ; c1ccc(-c2ccc(N(c3ccc(-c4ccc5c(c4)oc4cc(-c6ccccc6)c6ccccc6c45)cc3)c3cccc4sc5ccccc5c34)cc2)cc1]

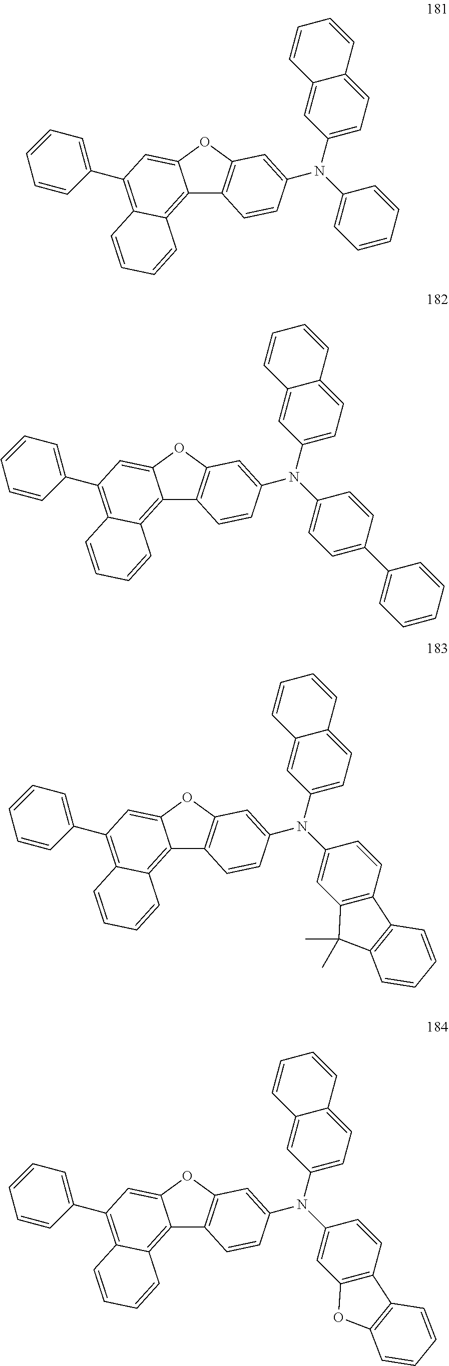 [SMILES: c1ccc(-c2cc3oc4cc(N(c5ccccc5)c5ccc6ccccc6c5)ccc4c3c3ccccc23)cc1 ; c1ccc(-c2ccc(N(c3ccc4ccccc4c3)c3ccc4c(c3)oc3cc(-c5ccccc5)c5ccccc5c34)cc2)cc1 ; CC1(C)c2ccccc2-c2ccc(N(c3ccc4ccccc4c3)c3ccc4c(c3)oc3cc(-c5ccccc5)c5ccccc5c34)cc21 ; c1ccc(-c2cc3oc4cc(N(c5ccc6ccccc6c5)c5ccc6c(c5)oc5ccccc56)ccc4c3c3ccccc23)cc1]

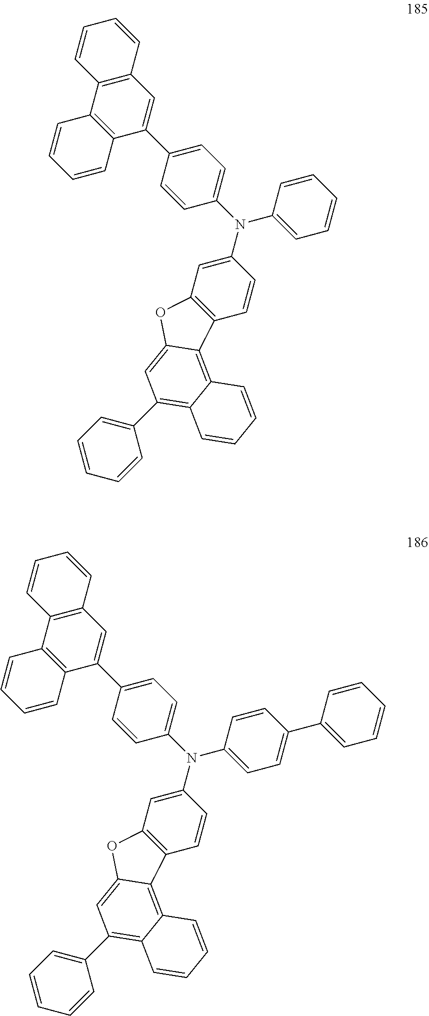 [SMILES: c1ccc(-c2cc3oc4cc(N(c5ccccc5)c5ccc(-c6cc7ccccc7c7ccccc67)cc5)ccc4c3c3ccccc23)cc1 ; c1ccc(-c2ccc(N(c3ccc(-c4cc5ccccc5c5ccccc45)cc3)c3ccc4c(c3)oc3cc(-c5ccccc5)c5ccccc5c34)cc2)cc1]

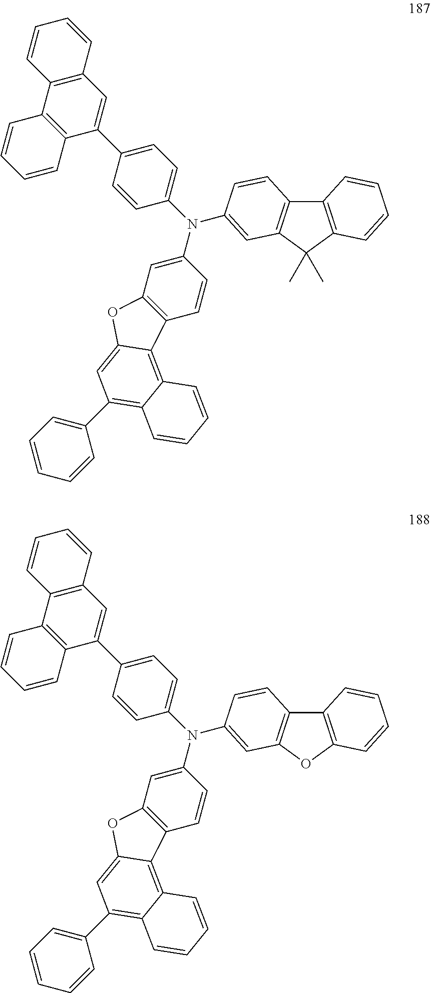 [SMILES: CC1(C)c2ccccc2-c2ccc(N(c3ccc(-c4cc5ccccc5c5ccccc45)cc3)c3ccc4c(c3)oc3cc(-c5ccccc5)c5ccccc5c34)cc21 ; c1ccc(-c2cc3oc4cc(N(c5ccc(-c6cc7ccccc7c7ccccc67)cc5)c5ccc6c(c5)oc5ccccc56)ccc4c3c3ccccc23)cc1]

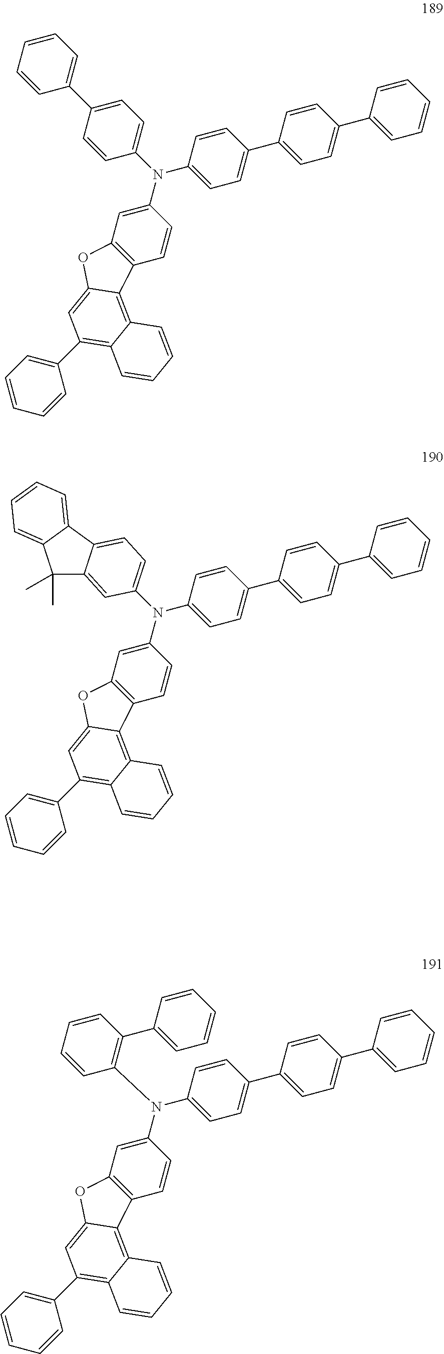 [SMILES: c1ccc(-c2ccc(-c3ccc(N(c4ccc(-c5ccccc5)cc4)c4ccc5c(c4)oc4cc(-c6ccccc6)c6ccccc6c45)cc3)cc2)cc1 ; CC1(C)c2ccccc2-c2ccc(N(c3ccc(-c4ccc(-c5ccccc5)cc4)cc3)c3ccc4c(c3)oc3cc(-c5ccccc5)c5ccccc5c34)cc21 ; c1ccc(-c2ccc(-c3ccc(N(c4ccc5c(c4)oc4cc(-c6ccccc6)c6ccccc6c45)c4ccccc4-c4ccccc4)cc3)cc2)cc1]

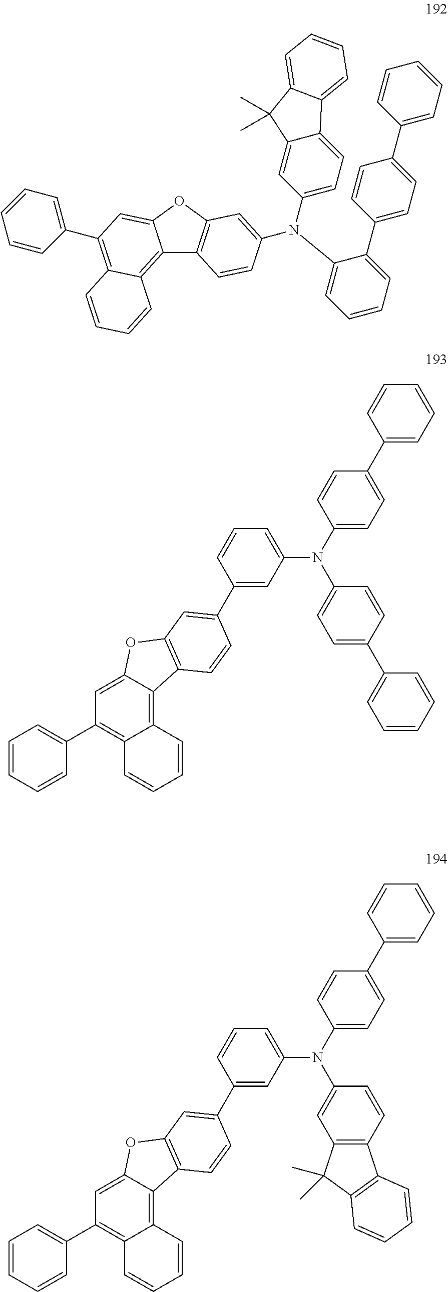 [SMILES: CC1(C)c2ccccc2-c2ccc(N(c3ccc4c(c3)oc3cc(-c5ccccc5)c5ccccc5c34)c3ccccc3-c3ccc(-c4ccccc4)cc3)cc21 ; c1ccc(-c2ccc(N(c3ccc(-c4ccccc4)cc3)c3cccc(-c4ccc5c(c4)oc4cc(-c6ccccc6)c6ccccc6c45)c3)cc2)cc1 ; CC1(C)c2ccccc2-c2ccc(N(c3ccc(-c4ccccc4)cc3)c3cccc(-c4ccc5c(c4)oc4cc(-c6ccccc6)c6ccccc6c45)c3)cc21]

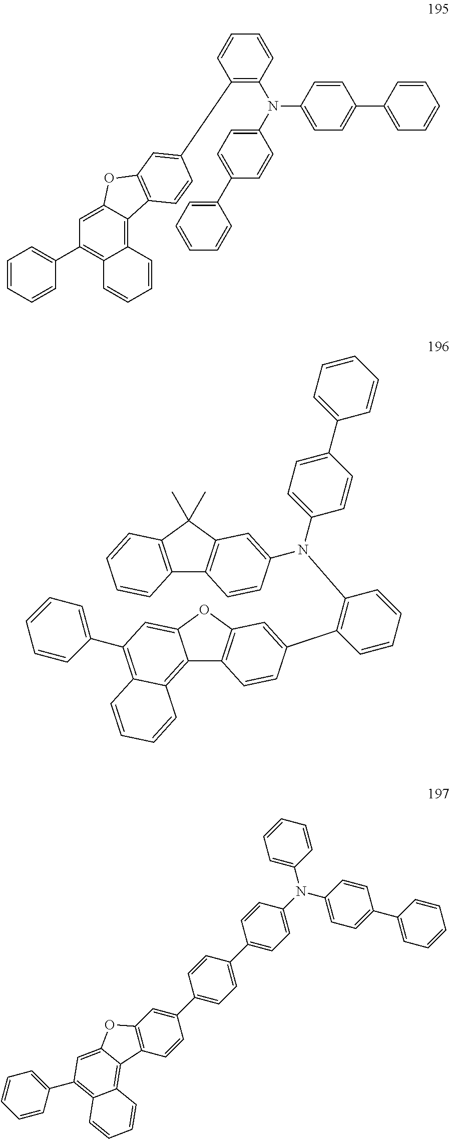 [SMILES: c1ccc(-c2ccc(N(c3ccc(-c4ccccc4)cc3)c3ccccc3-c3ccc4c(c3)oc3cc(-c5ccccc5)c5ccccc5c34)cc2)cc1 ; CC1(C)c2ccccc2-c2ccc(N(c3ccc(-c4ccccc4)cc3)c3ccccc3-c3ccc4c(c3)oc3cc(-c5ccccc5)c5ccccc5c34)cc21 ; c1ccc(-c2ccc(N(c3ccccc3)c3ccc(-c4ccc(-c5ccc6c(c5)oc5cc(-c7ccccc7)c7ccccc7c56)cc4)cc3)cc2)cc1]

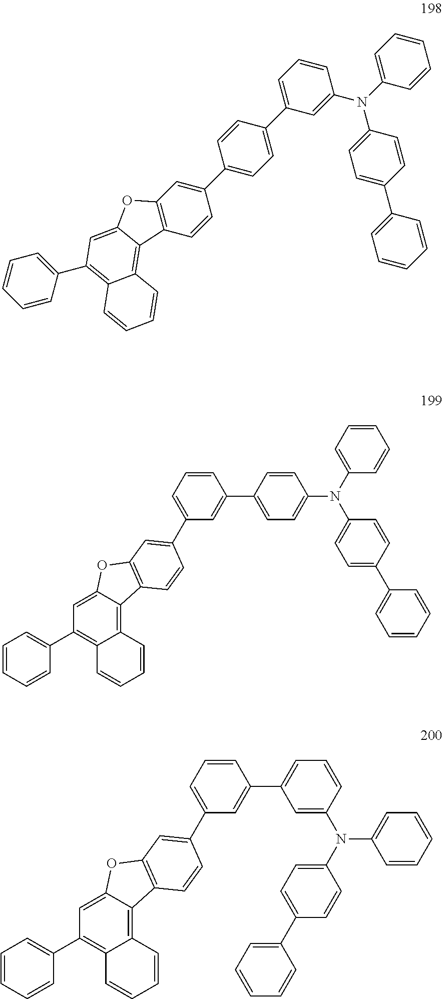 [SMILES: c1ccc(-c2ccc(N(c3ccccc3)c3cccc(-c4ccc(-c5ccc6c(c5)oc5cc(-c7ccccc7)c7ccccc7c56)cc4)c3)cc2)cc1 ; c1ccc(-c2ccc(N(c3ccccc3)c3ccc(-c4cccc(-c5ccc6c(c5)oc5cc(-c7ccccc7)c7ccccc7c56)c4)cc3)cc2)cc1 ; c1ccc(-c2ccc(N(c3ccccc3)c3cccc(-c4cccc(-c5ccc6c(c5)oc5cc(-c7ccccc7)c7ccccc7c56)c4)c3)cc2)cc1]

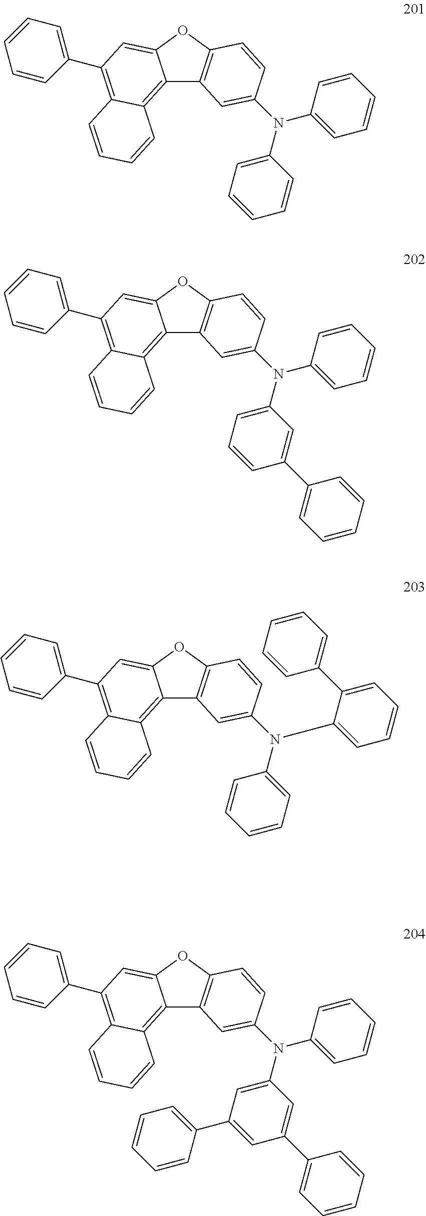 [SMILES: c1ccc(-c2cc3oc4ccc(N(c5ccccc5)c5ccccc5)cc4c3c3ccccc23)cc1 ; c1ccc(-c2cccc(N(c3ccccc3)c3ccc4oc5cc(-c6ccccc6)c6ccccc6c5c4c3)c2)cc1 ; c1ccc(-c2ccccc2N(c2ccccc2)c2ccc3oc4cc(-c5ccccc5)c5ccccc5c4c3c2)cc1 ; c1ccc(-c2cc(-c3ccccc3)cc(N(c3ccccc3)c3ccc4oc5cc(-c6ccccc6)c6ccccc6c5c4c3)c2)cc1]

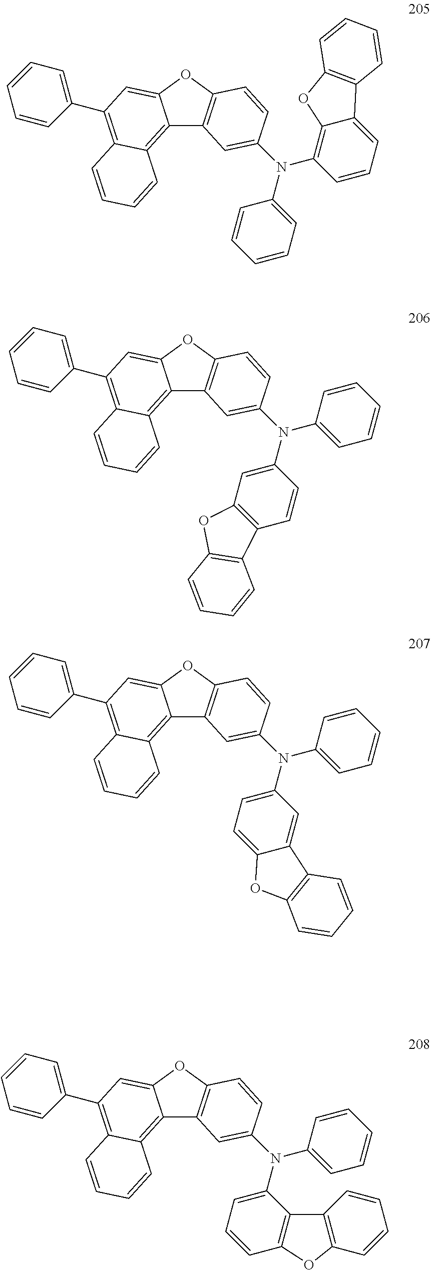 [SMILES: c1ccc(-c2cc3oc4ccc(N(c5ccccc5)c5cccc6c5oc5ccccc56)cc4c3c3ccccc23)cc1 ; c1ccc(-c2cc3oc4ccc(N(c5ccccc5)c5ccc6c(c5)oc5ccccc56)cc4c3c3ccccc23)cc1 ; c1ccc(-c2cc3oc4ccc(N(c5ccccc5)c5ccc6oc7ccccc7c6c5)cc4c3c3ccccc23)cc1 ; c1ccc(-c2cc3oc4ccc(N(c5ccccc5)c5cccc6oc7ccccc7c56)cc4c3c3ccccc23)cc1]

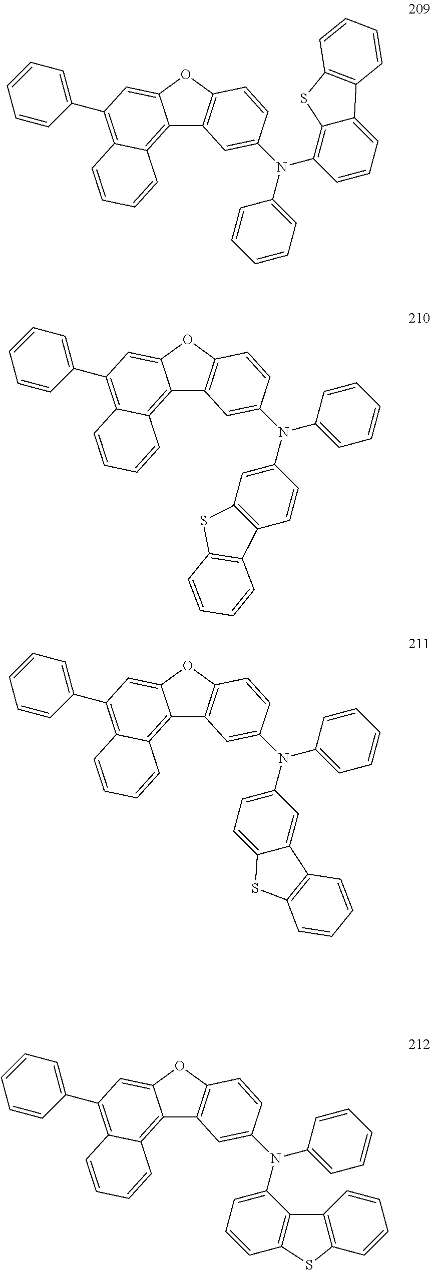 [SMILES: c1ccc(-c2cc3oc4ccc(N(c5ccccc5)c5cccc6c5sc5ccccc56)cc4c3c3ccccc23)cc1 ; c1ccc(-c2cc3oc4ccc(N(c5ccccc5)c5ccc6c(c5)sc5ccccc56)cc4c3c3ccccc23)cc1 ; c1ccc(-c2cc3oc4ccc(N(c5ccccc5)c5ccc6sc7ccccc7c6c5)cc4c3c3ccccc23)cc1 ; c1ccc(-c2cc3oc4ccc(N(c5ccccc5)c5cccc6sc7ccccc7c56)cc4c3c3ccccc23)cc1]

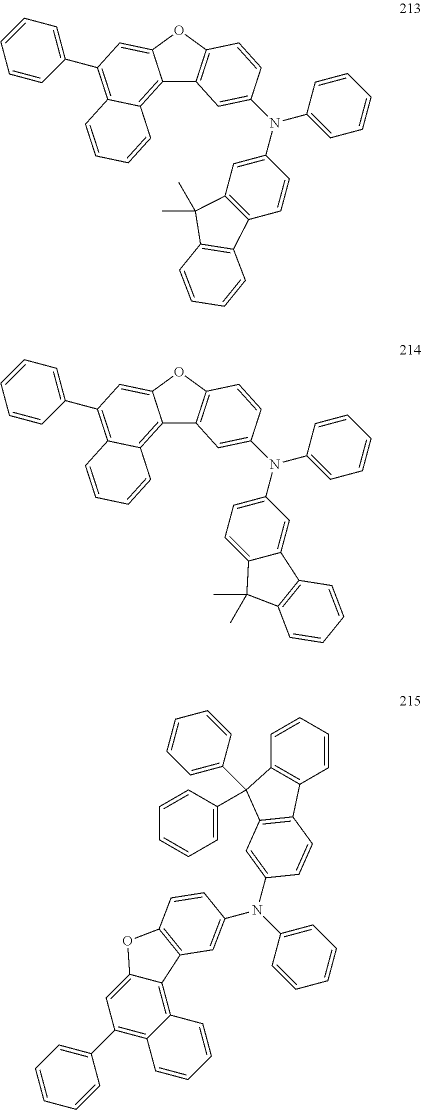 [SMILES: CC1(C)c2ccccc2-c2ccc(N(c3ccccc3)c3ccc4oc5cc(-c6ccccc6)c6ccccc6c5c4c3)cc21 ; CC1(C)c2ccccc2-c2cc(N(c3ccccc3)c3ccc4oc5cc(-c6ccccc6)c6ccccc6c5c4c3)ccc21 ; c1ccc(-c2cc3oc4ccc(N(c5ccccc5)c5ccc6c(c5)C(c5ccccc5)(c5ccccc5)c5ccccc5-6)cc4c3c3ccccc23)cc1]

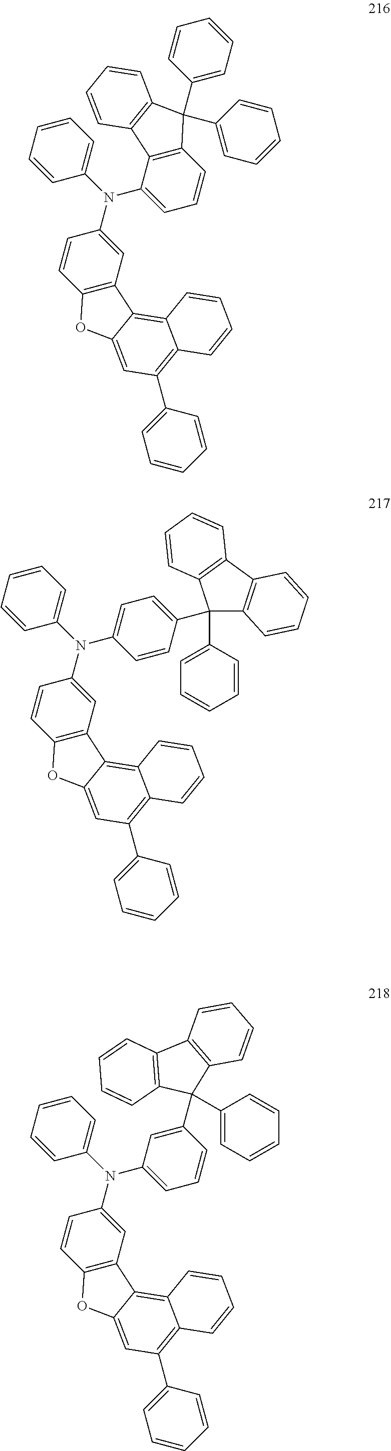

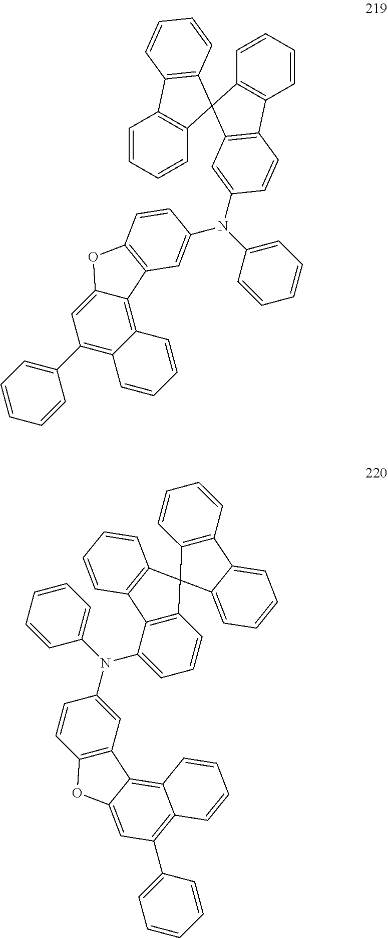 [SMILES: c1ccc(-c2cc3oc4ccc(N(c5ccccc5)c5ccc6c(c5)C5(c7ccccc7-c7ccccc75)c5ccccc5-6)cc4c3c3ccccc23)cc1 ; c1ccc(-c2cc3oc4ccc(N(c5ccccc5)c5cccc6c5-c5ccccc5C65c6ccccc6-c6ccccc65)cc4c3c3ccccc23)cc1]

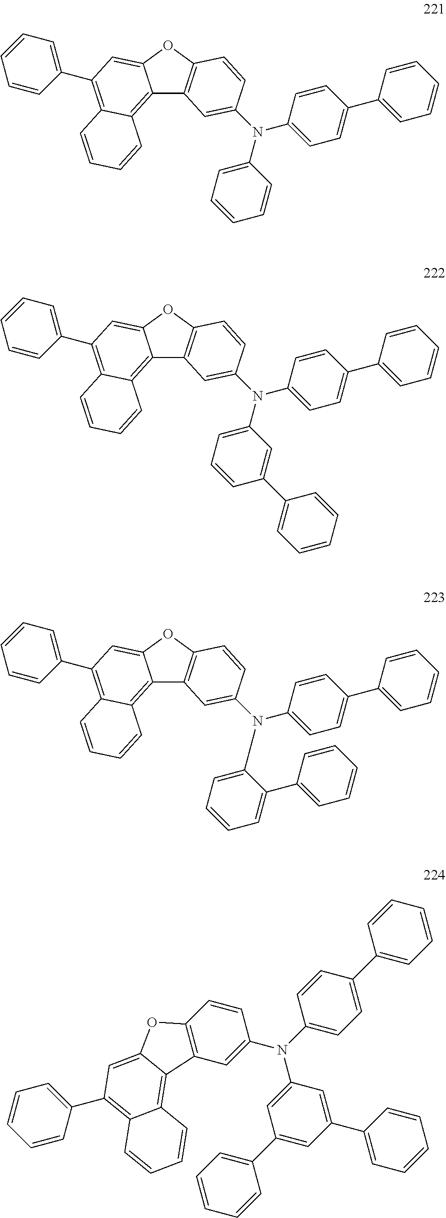 [SMILES: c1ccc(-c2ccc(N(c3ccccc3)c3ccc4oc5cc(-c6ccccc6)c6ccccc6c5c4c3)cc2)cc1 ; c1ccc(-c2ccc(N(c3cccc(-c4ccccc4)c3)c3ccc4oc5cc(-c6ccccc6)c6ccccc6c5c4c3)cc2)cc1 ; c1ccc(-c2ccc(N(c3ccc4oc5cc(-c6ccccc6)c6ccccc6c5c4c3)c3ccccc3-c3ccccc3)cc2)cc1 ; c1ccc(-c2ccc(N(c3cc(-c4ccccc4)cc(-c4ccccc4)c3)c3ccc4oc5cc(-c6ccccc6)c6ccccc6c5c4c3)cc2)cc1]

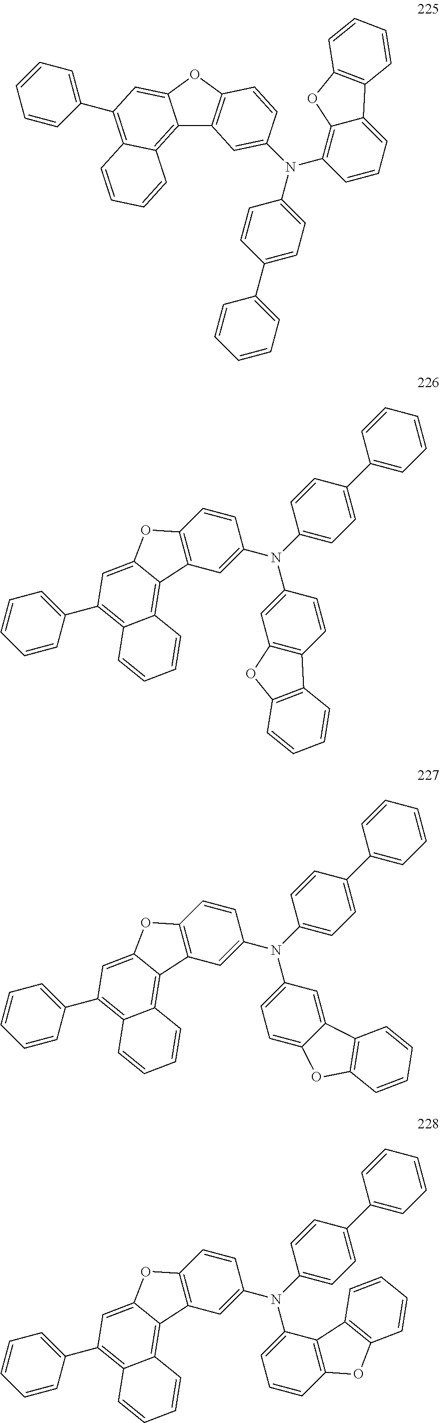 [SMILES: c1ccc(-c2ccc(N(c3ccc4oc5cc(-c6ccccc6)c6ccccc6c5c4c3)c3cccc4c3oc3ccccc34)cc2)cc1 ; c1ccc(-c2ccc(N(c3ccc4c(c3)oc3ccccc34)c3ccc4oc5cc(-c6ccccc6)c6ccccc6c5c4c3)cc2)cc1 ; c1ccc(-c2ccc(N(c3ccc4oc5ccccc5c4c3)c3ccc4oc5cc(-c6ccccc6)c6ccccc6c5c4c3)cc2)cc1 ; c1ccc(-c2ccc(N(c3ccc4oc5cc(-c6ccccc6)c6ccccc6c5c4c3)c3cccc4oc5ccccc5c34)cc2)cc1]

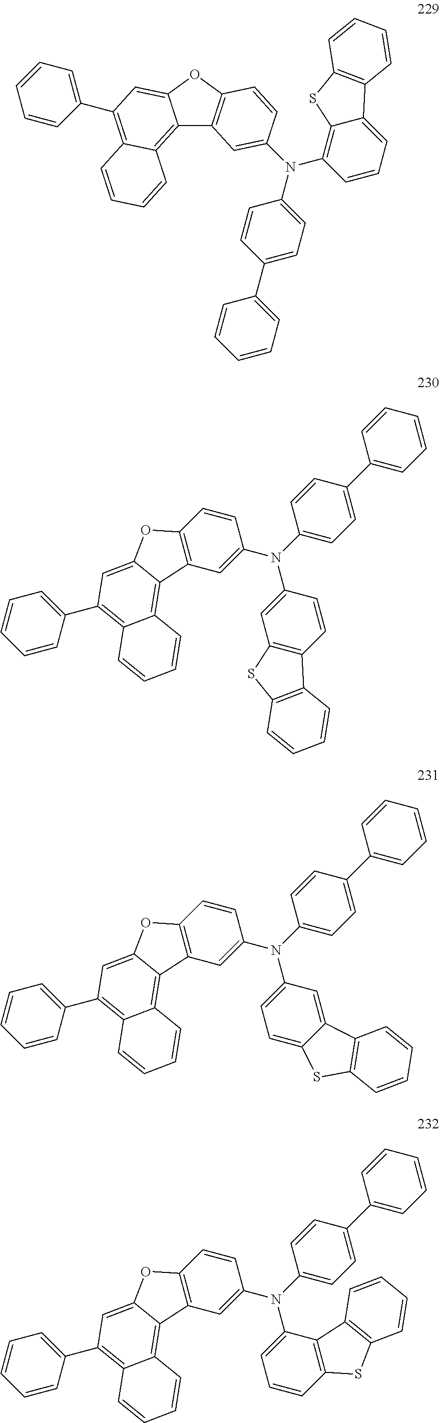 [SMILES: c1ccc(-c2ccc(N(c3ccc4oc5cc(-c6ccccc6)c6ccccc6c5c4c3)c3cccc4c3sc3ccccc34)cc2)cc1 ; c1ccc(-c2ccc(N(c3ccc4c(c3)sc3ccccc34)c3ccc4oc5cc(-c6ccccc6)c6ccccc6c5c4c3)cc2)cc1 ; c1ccc(-c2ccc(N(c3ccc4sc5ccccc5c4c3)c3ccc4oc5cc(-c6ccccc6)c6ccccc6c5c4c3)cc2)cc1 ; c1ccc(-c2ccc(N(c3ccc4oc5cc(-c6ccccc6)c6ccccc6c5c4c3)c3cccc4sc5ccccc5c34)cc2)cc1]

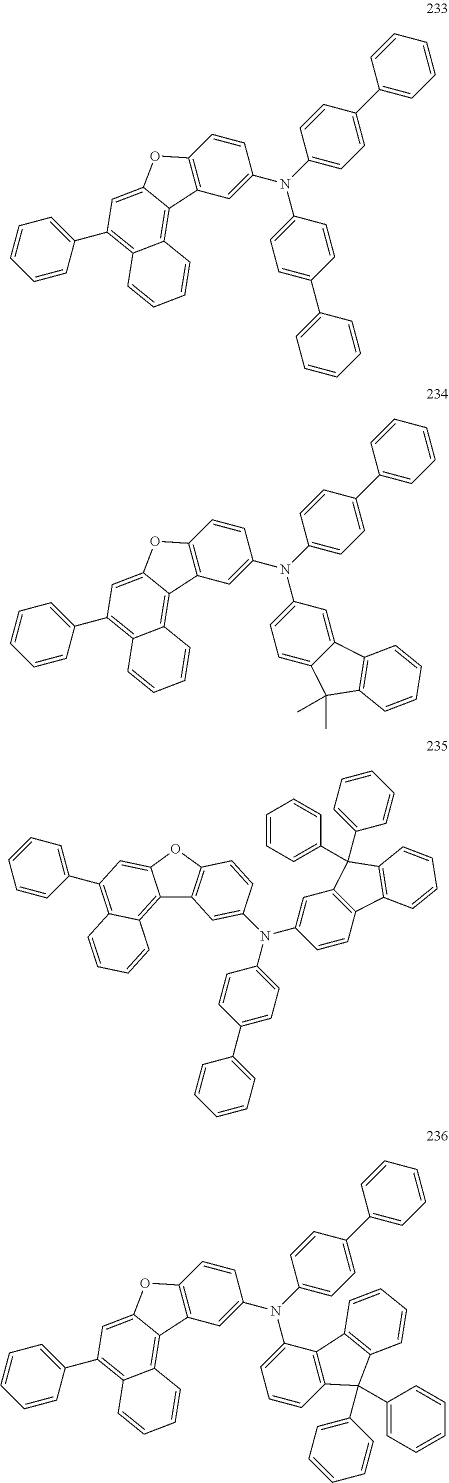 [SMILES: c1ccc(-c2ccc(N(c3ccc(-c4ccccc4)cc3)c3ccc4oc5cc(-c6ccccc6)c6ccccc6c5c4c3)cc2)cc1 ; CC1(C)c2ccccc2-c2cc(N(c3ccc(-c4ccccc4)cc3)c3ccc4oc5cc(-c6ccccc6)c6ccccc6c5c4c3)ccc21 ; c1ccc(-c2ccc(N(c3ccc4c(c3)C(c3ccccc3)(c3ccccc3)c3ccccc3-4)c3ccc4oc5cc(-c6ccccc6)c6ccccc6c5c4c3)cc2)cc1 ; c1ccc(-c2ccc(N(c3ccc4oc5cc(-c6ccccc6)c6ccccc6c5c4c3)c3cccc4c3-c3ccccc3C4(c3ccccc3)c3ccccc3)cc2)cc1]

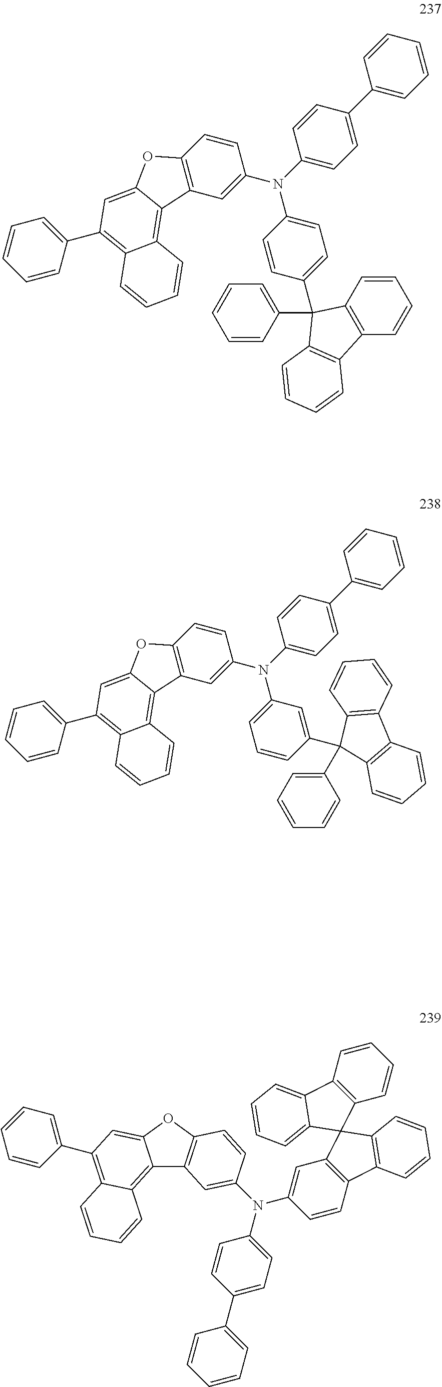 [SMILES: c1ccc(-c2ccc(N(c3ccc(C4(c5ccccc5)c5ccccc5-c5ccccc54)cc3)c3ccc4oc5cc(-c6ccccc6)c6ccccc6c5c4c3)cc2)cc1 ; c1ccc(-c2ccc(N(c3cccc(C4(c5ccccc5)c5ccccc5-c5ccccc54)c3)c3ccc4oc5cc(-c6ccccc6)c6ccccc6c5c4c3)cc2)cc1 ; c1ccc(-c2ccc(N(c3ccc4c(c3)C3(c5ccccc5-c5ccccc53)c3ccccc3-4)c3ccc4oc5cc(-c6ccccc6)c6ccccc6c5c4c3)cc2)cc1]

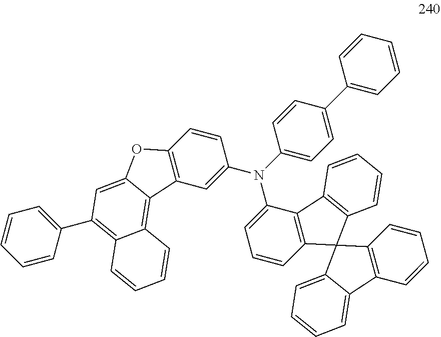 [SMILES: c1ccc(-c2ccc(N(c3ccc4oc5cc(-c6ccccc6)c6ccccc6c5c4c3)c3cccc4c3-c3ccccc3C43c4ccccc4-c4ccccc43)cc2)cc1]

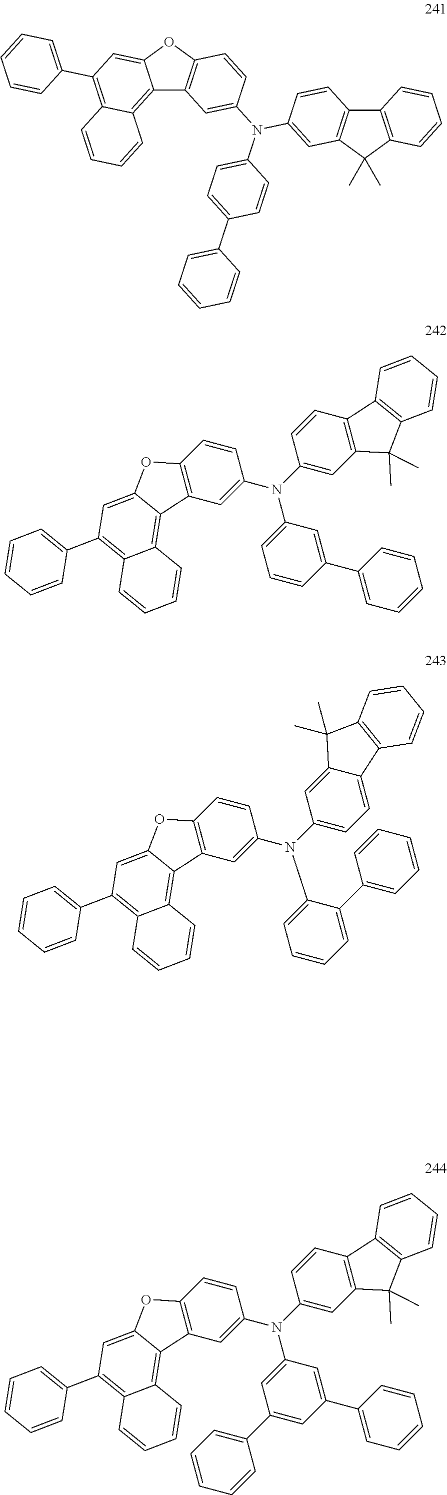 [SMILES: CC1(C)c2ccccc2-c2ccc(N(c3ccc(-c4ccccc4)cc3)c3ccc4oc5cc(-c6ccccc6)c6ccccc6c5c4c3)cc21 ; CC1(C)c2ccccc2-c2ccc(N(c3cccc(-c4ccccc4)c3)c3ccc4oc5cc(-c6ccccc6)c6ccccc6c5c4c3)cc21 ; CC1(C)c2ccccc2-c2ccc(N(c3ccc4oc5cc(-c6ccccc6)c6ccccc6c5c4c3)c3ccccc3-c3ccccc3)cc21 ; CC1(C)c2ccccc2-c2ccc(N(c3cc(-c4ccccc4)cc(-c4ccccc4)c3)c3ccc4oc5cc(-c6ccccc6)c6ccccc6c5c4c3)cc21]

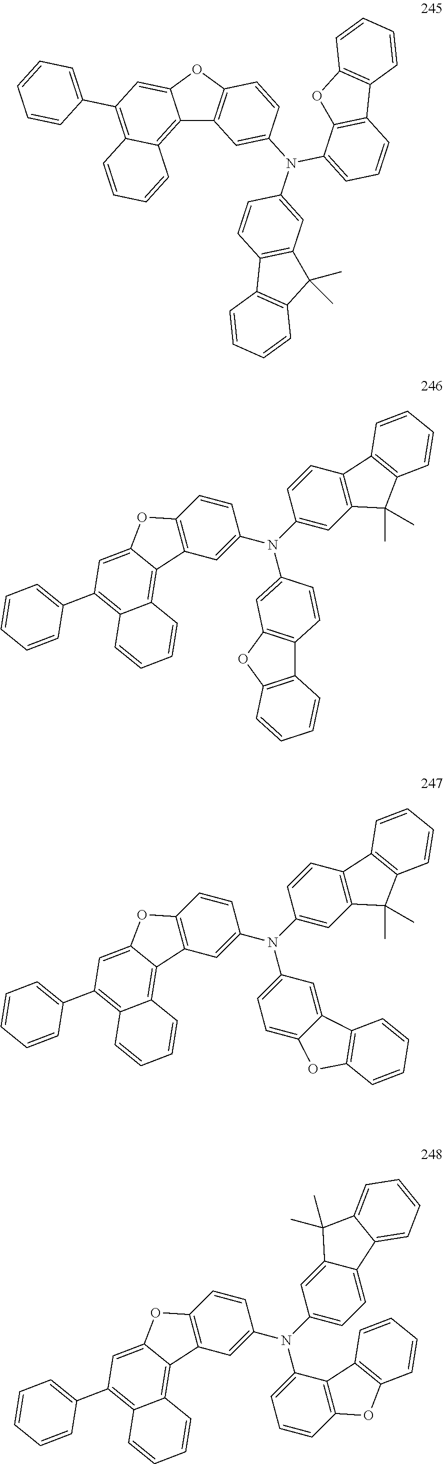 [SMILES: CC1(C)c2ccccc2-c2ccc(N(c3ccc4oc5cc(-c6ccccc6)c6ccccc6c5c4c3)c3cccc4c3oc3ccccc34)cc21 ; CC1(C)c2ccccc2-c2ccc(N(c3ccc4c(c3)oc3ccccc34)c3ccc4oc5cc(-c6ccccc6)c6ccccc6c5c4c3)cc21 ; CC1(C)c2ccccc2-c2ccc(N(c3ccc4oc5ccccc5c4c3)c3ccc4oc5cc(-c6ccccc6)c6ccccc6c5c4c3)cc21 ; CC1(C)c2ccccc2-c2ccc(N(c3ccc4oc5cc(-c6ccccc6)c6ccccc6c5c4c3)c3cccc4oc5ccccc5c34)cc21]

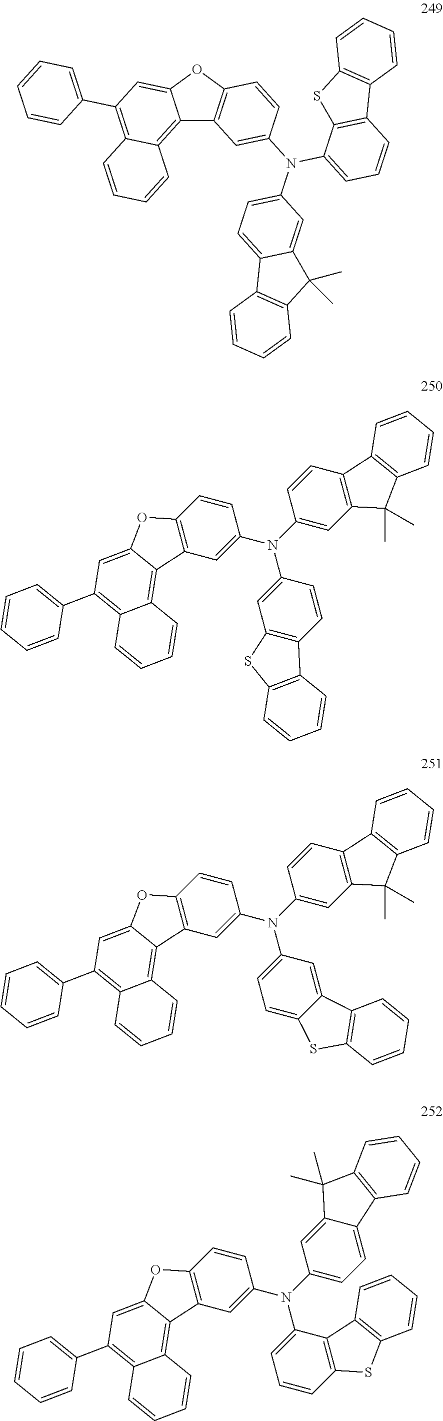 [SMILES: CC1(C)c2ccccc2-c2ccc(N(c3ccc4oc5cc(-c6ccccc6)c6ccccc6c5c4c3)c3cccc4c3sc3ccccc34)cc21 ; CC1(C)c2ccccc2-c2ccc(N(c3ccc4c(c3)sc3ccccc34)c3ccc4oc5cc(-c6ccccc6)c6ccccc6c5c4c3)cc21 ; CC1(C)c2ccccc2-c2ccc(N(c3ccc4sc5ccccc5c4c3)c3ccc4oc5cc(-c6ccccc6)c6ccccc6c5c4c3)cc21 ; CC1(C)c2ccccc2-c2ccc(N(c3ccc4oc5cc(-c6ccccc6)c6ccccc6c5c4c3)c3cccc4sc5ccccc5c34)cc21]

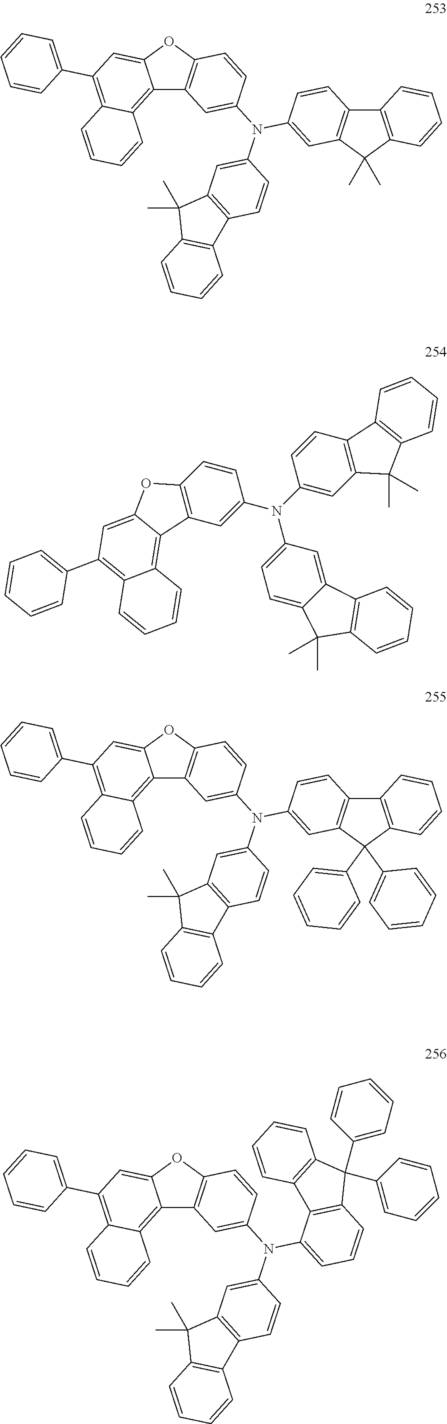 [SMILES: CC1(C)c2ccccc2-c2ccc(N(c3ccc4c(c3)C(C)(C)c3ccccc3-4)c3ccc4oc5cc(-c6ccccc6)c6ccccc6c5c4c3)cc21 ; CC1(C)c2ccccc2-c2cc(N(c3ccc4c(c3)C(C)(C)c3ccccc3-4)c3ccc4oc5cc(-c6ccccc6)c6ccccc6c5c4c3)ccc21 ; CC1(C)c2ccccc2-c2ccc(N(c3ccc4c(c3)C(c3ccccc3)(c3ccccc3)c3ccccc3-4)c3ccc4oc5cc(-c6ccccc6)c6ccccc6c5c4c3)cc21 ; CC1(C)c2ccccc2-c2ccc(N(c3ccc4oc5cc(-c6ccccc6)c6ccccc6c5c4c3)c3cccc4c3-c3ccccc3C4(c3ccccc3)c3ccccc3)cc21]

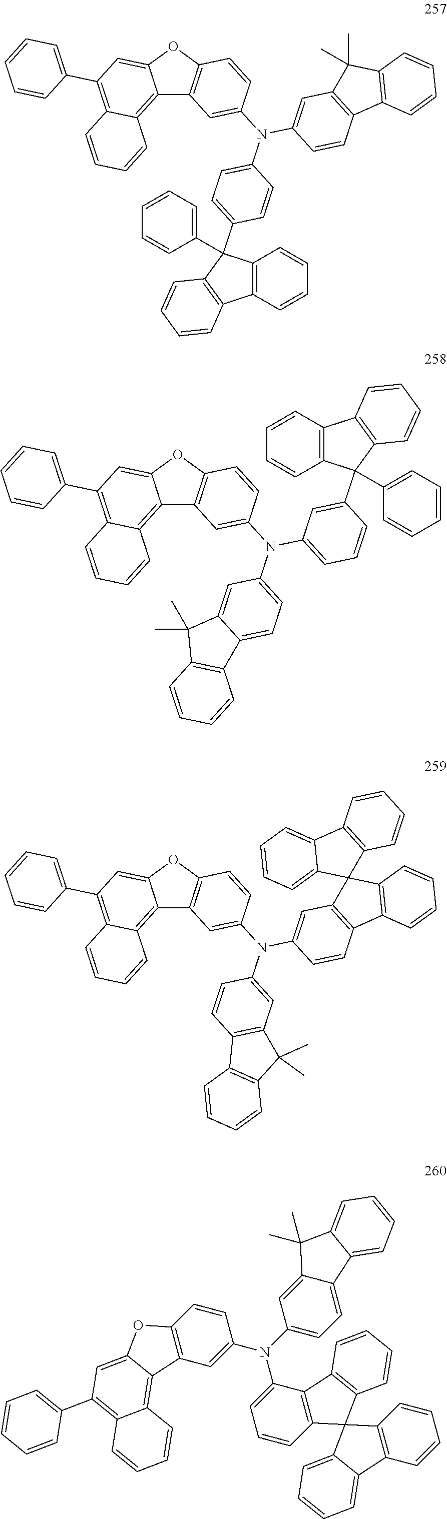 [SMILES: CC1(C)c2ccccc2-c2ccc(N(c3ccc(C4(c5ccccc5)c5ccccc5-c5ccccc54)cc3)c3ccc4oc5cc(-c6ccccc6)c6ccccc6c5c4c3)cc21 ; CC1(C)c2ccccc2-c2ccc(N(c3cccc(C4(c5ccccc5)c5ccccc5-c5ccccc54)c3)c3ccc4oc5cc(-c6ccccc6)c6ccccc6c5c4c3)cc21 ; CC1(C)c2ccccc2-c2ccc(N(c3ccc4c(c3)C3(c5ccccc5-c5ccccc53)c3ccccc3-4)c3ccc4oc5cc(-c6ccccc6)c6ccccc6c5c4c3)cc21 ; CC1(C)c2ccccc2-c2ccc(N(c3ccc4oc5cc(-c6ccccc6)c6ccccc6c5c4c3)c3cccc4c3-c3ccccc3C43c4ccccc4-c4ccccc43)cc21]

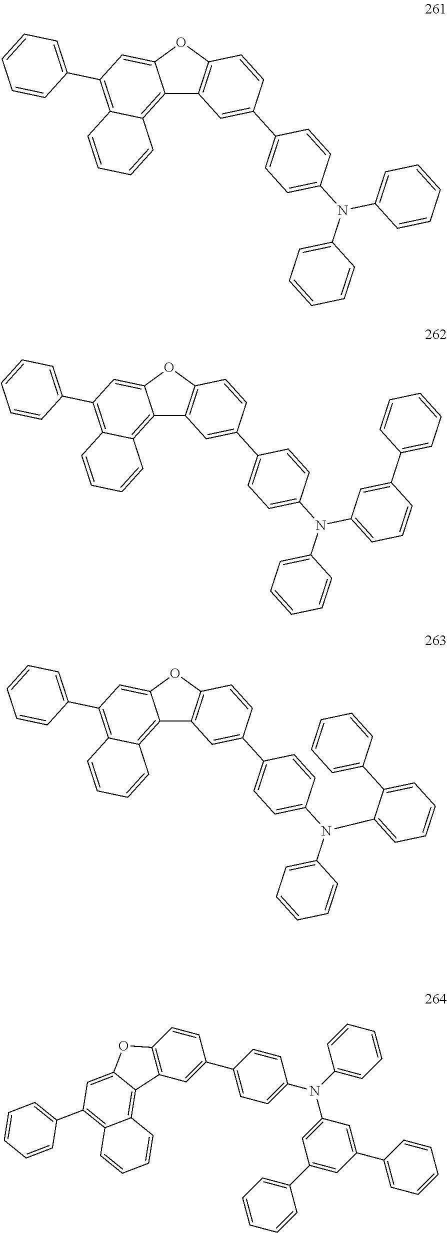 [SMILES: c1ccc(-c2cc3oc4ccc(-c5ccc(N(c6ccccc6)c6ccccc6)cc5)cc4c3c3ccccc23)cc1 ; c1ccc(-c2cccc(N(c3ccccc3)c3ccc(-c4ccc5oc6cc(-c7ccccc7)c7ccccc7c6c5c4)cc3)c2)cc1 ; c1ccc(-c2ccccc2N(c2ccccc2)c2ccc(-c3ccc4oc5cc(-c6ccccc6)c6ccccc6c5c4c3)cc2)cc1 ; c1ccc(-c2cc(-c3ccccc3)cc(N(c3ccccc3)c3ccc(-c4ccc5oc6cc(-c7ccccc7)c7ccccc7c6c5c4)cc3)c2)cc1]

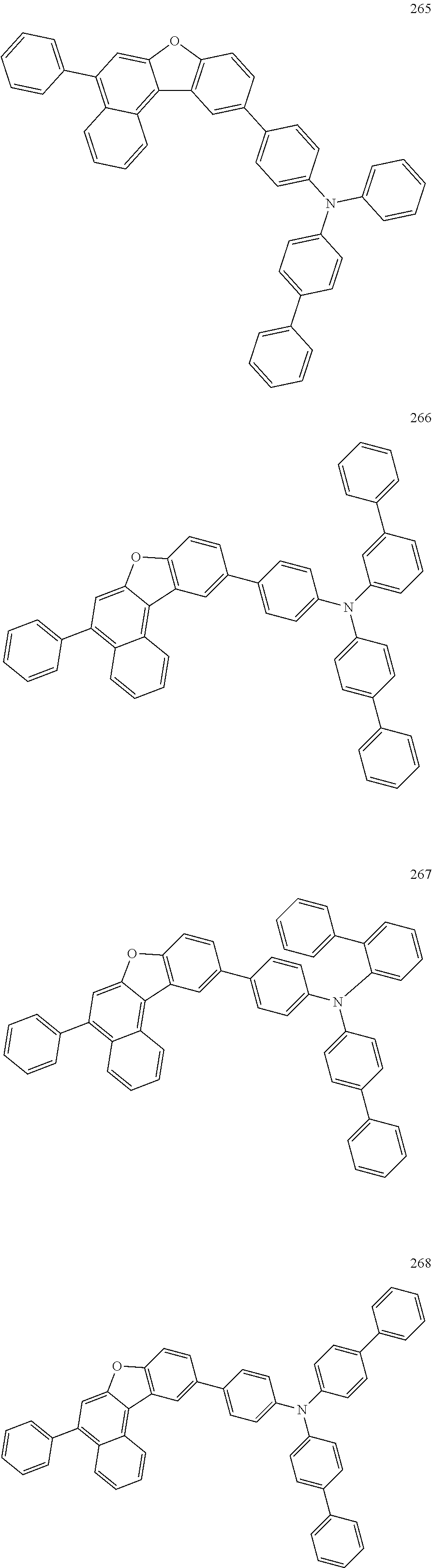 [SMILES: c1ccc(-c2ccc(N(c3ccccc3)c3ccc(-c4ccc5oc6cc(-c7ccccc7)c7ccccc7c6c5c4)cc3)cc2)cc1 ; c1ccc(-c2ccc(N(c3ccc(-c4ccc5oc6cc(-c7ccccc7)c7ccccc7c6c5c4)cc3)c3cccc(-c4ccccc4)c3)cc2)cc1 ; c1ccc(-c2ccc(N(c3ccc(-c4ccc5oc6cc(-c7ccccc7)c7ccccc7c6c5c4)cc3)c3ccccc3-c3ccccc3)cc2)cc1 ; c1ccc(-c2ccc(N(c3ccc(-c4ccccc4)cc3)c3ccc(-c4ccc5oc6cc(-c7ccccc7)c7ccccc7c6c5c4)cc3)cc2)cc1]

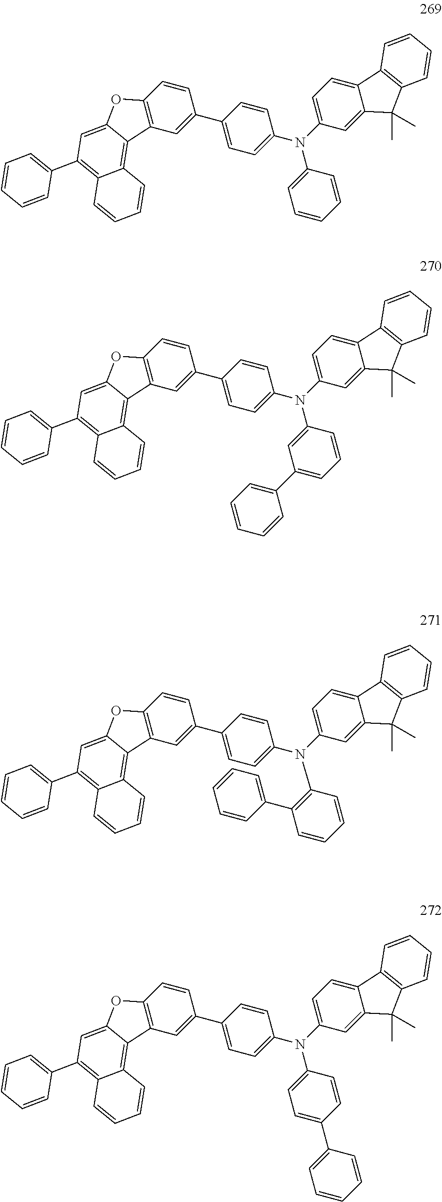 [SMILES: CC1(C)c2ccccc2-c2ccc(N(c3ccccc3)c3ccc(-c4ccc5oc6cc(-c7ccccc7)c7ccccc7c6c5c4)cc3)cc21 ; CC1(C)c2ccccc2-c2ccc(N(c3ccc(-c4ccc5oc6cc(-c7ccccc7)c7ccccc7c6c5c4)cc3)c3cccc(-c4ccccc4)c3)cc21 ; CC1(C)c2ccccc2-c2ccc(N(c3ccc(-c4ccc5oc6cc(-c7ccccc7)c7ccccc7c6c5c4)cc3)c3ccccc3-c3ccccc3)cc21 ; CC1(C)c2ccccc2-c2ccc(N(c3ccc(-c4ccccc4)cc3)c3ccc(-c4ccc5oc6cc(-c7ccccc7)c7ccccc7c6c5c4)cc3)cc21]

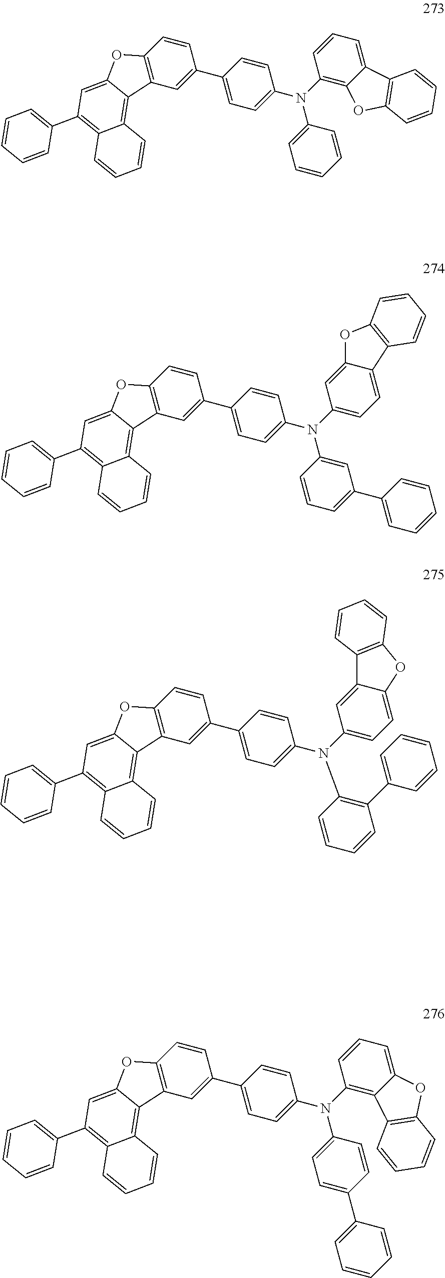 [SMILES: c1ccc(-c2cc3oc4ccc(-c5ccc(N(c6ccccc6)c6cccc7c6oc6ccccc67)cc5)cc4c3c3ccccc23)cc1 ; c1ccc(-c2cccc(N(c3ccc(-c4ccc5oc6cc(-c7ccccc7)c7ccccc7c6c5c4)cc3)c3ccc4c(c3)oc3ccccc34)c2)cc1 ; c1ccc(-c2ccccc2N(c2ccc(-c3ccc4oc5cc(-c6ccccc6)c6ccccc6c5c4c3)cc2)c2ccc3oc4ccccc4c3c2)cc1 ; c1ccc(-c2ccc(N(c3ccc(-c4ccc5oc6cc(-c7ccccc7)c7ccccc7c6c5c4)cc3)c3cccc4oc5ccccc5c34)cc2)cc1]

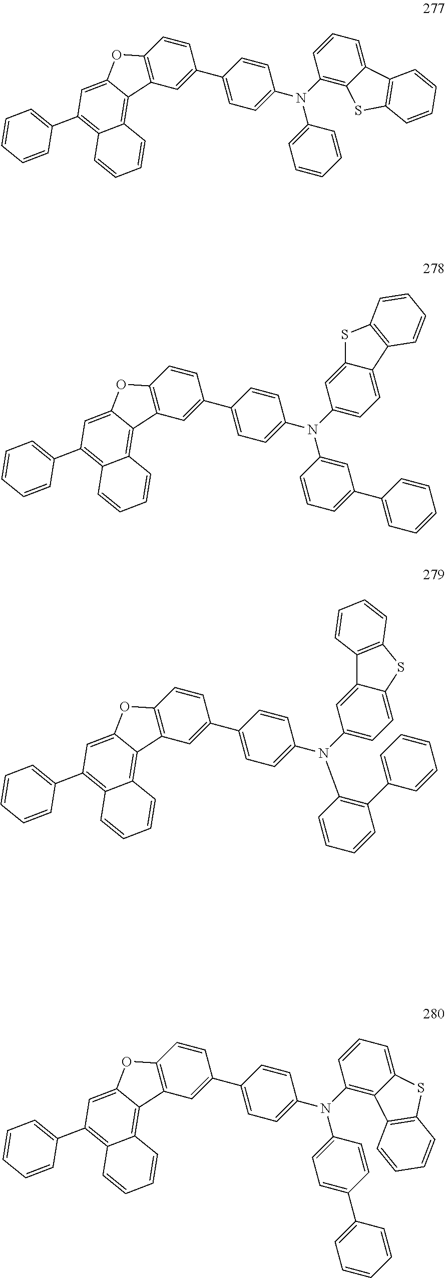 [SMILES: c1ccc(-c2cc3oc4ccc(-c5ccc(N(c6ccccc6)c6cccc7c6sc6ccccc67)cc5)cc4c3c3ccccc23)cc1 ; c1ccc(-c2cccc(N(c3ccc(-c4ccc5oc6cc(-c7ccccc7)c7ccccc7c6c5c4)cc3)c3ccc4c(c3)sc3ccccc34)c2)cc1 ; c1ccc(-c2ccccc2N(c2ccc(-c3ccc4oc5cc(-c6ccccc6)c6ccccc6c5c4c3)cc2)c2ccc3sc4ccccc4c3c2)cc1 ; c1ccc(-c2ccc(N(c3ccc(-c4ccc5oc6cc(-c7ccccc7)c7ccccc7c6c5c4)cc3)c3cccc4sc5ccccc5c34)cc2)cc1]

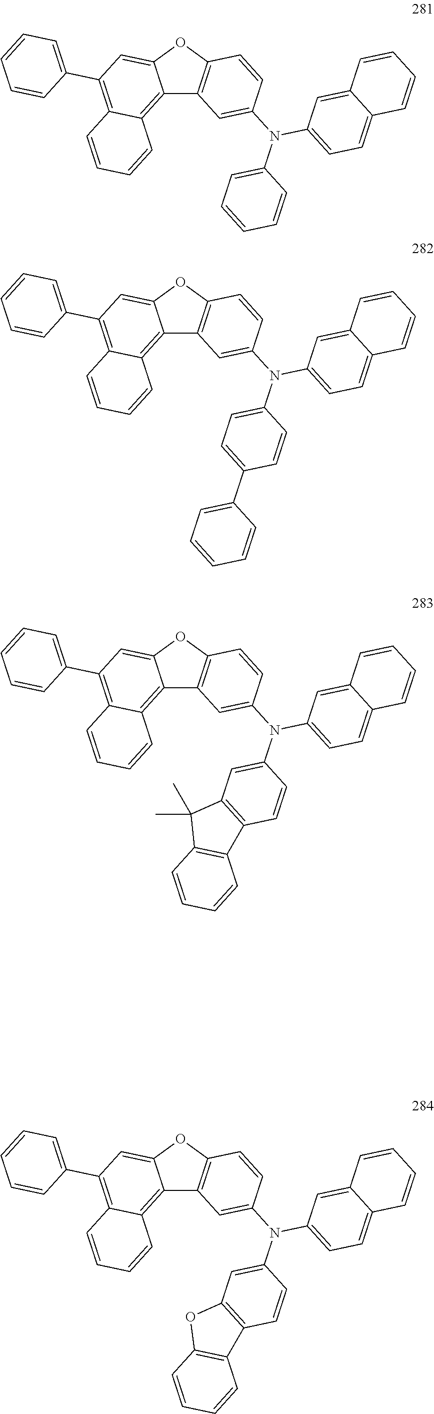 [SMILES: c1ccc(-c2cc3oc4ccc(N(c5ccccc5)c5ccc6ccccc6c5)cc4c3c3ccccc23)cc1 ; c1ccc(-c2ccc(N(c3ccc4ccccc4c3)c3ccc4oc5cc(-c6ccccc6)c6ccccc6c5c4c3)cc2)cc1 ; CC1(C)c2ccccc2-c2ccc(N(c3ccc4ccccc4c3)c3ccc4oc5cc(-c6ccccc6)c6ccccc6c5c4c3)cc21 ; c1ccc(-c2cc3oc4ccc(N(c5ccc6ccccc6c5)c5ccc6c(c5)oc5ccccc56)cc4c3c3ccccc23)cc1]

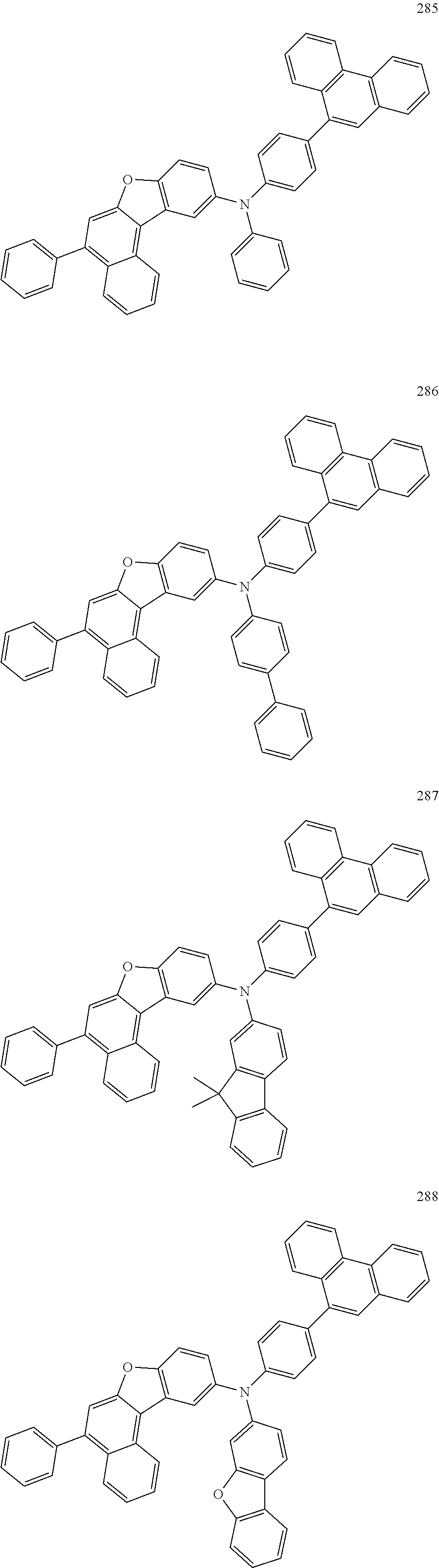 [SMILES: c1ccc(-c2cc3oc4ccc(N(c5ccccc5)c5ccc(-c6cc7ccccc7c7ccccc67)cc5)cc4c3c3ccccc23)cc1 ; c1ccc(-c2ccc(N(c3ccc(-c4cc5ccccc5c5ccccc45)cc3)c3ccc4oc5cc(-c6ccccc6)c6ccccc6c5c4c3)cc2)cc1 ; CC1(C)c2ccccc2-c2ccc(N(c3ccc(-c4cc5ccccc5c5ccccc45)cc3)c3ccc4oc5cc(-c6ccccc6)c6ccccc6c5c4c3)cc21 ; c1ccc(-c2cc3oc4ccc(N(c5ccc(-c6cc7ccccc7c7ccccc67)cc5)c5ccc6c(c5)oc5ccccc56)cc4c3c3ccccc23)cc1]

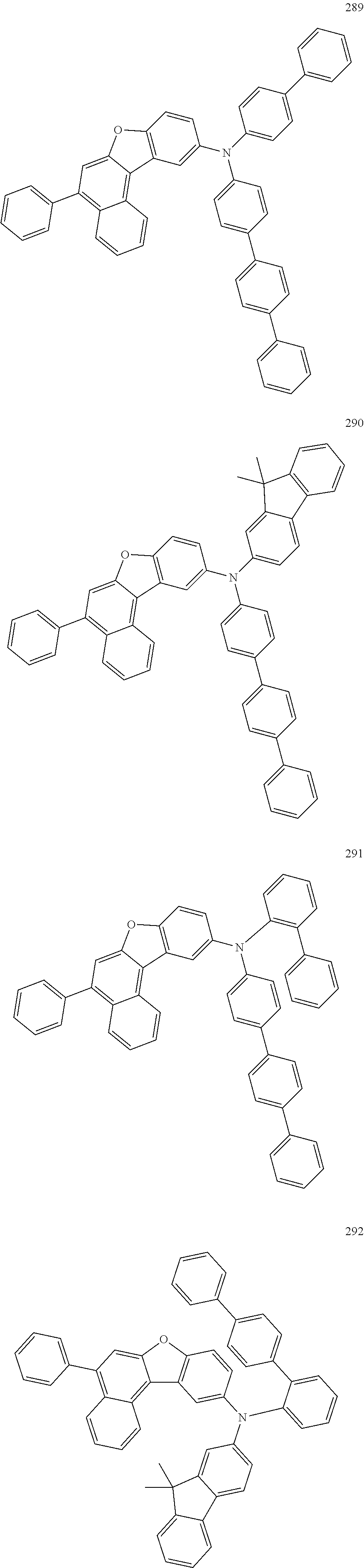 [SMILES: c1ccc(-c2ccc(-c3ccc(N(c4ccc(-c5ccccc5)cc4)c4ccc5oc6cc(-c7ccccc7)c7ccccc7c6c5c4)cc3)cc2)cc1 ; CC1(C)c2ccccc2-c2ccc(N(c3ccc(-c4ccc(-c5ccccc5)cc4)cc3)c3ccc4oc5cc(-c6ccccc6)c6ccccc6c5c4c3)cc21 ; c1ccc(-c2ccc(-c3ccc(N(c4ccc5oc6cc(-c7ccccc7)c7ccccc7c6c5c4)c4ccccc4-c4ccccc4)cc3)cc2)cc1 ; CC1(C)c2ccccc2-c2ccc(N(c3ccc4oc5cc(-c6ccccc6)c6ccccc6c5c4c3)c3ccccc3-c3ccc(-c4ccccc4)cc3)cc21]

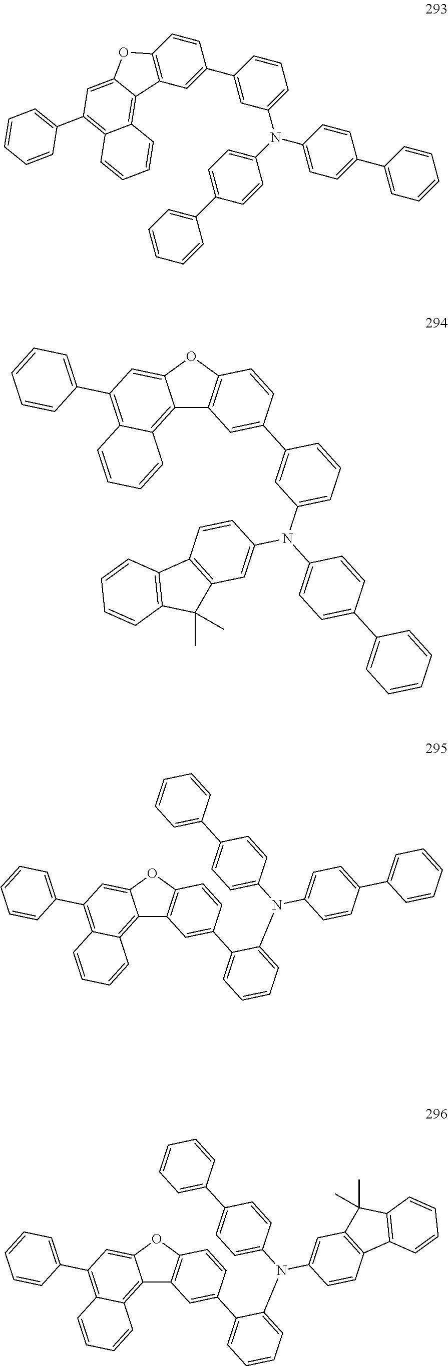 [SMILES: c1ccc(-c2ccc(N(c3ccc(-c4ccccc4)cc3)c3cccc(-c4ccc5oc6cc(-c7ccccc7)c7ccccc7c6c5c4)c3)cc2)cc1 ; CC1(C)c2ccccc2-c2ccc(N(c3ccc(-c4ccccc4)cc3)c3cccc(-c4ccc5oc6cc(-c7ccccc7)c7ccccc7c6c5c4)c3)cc21 ; c1ccc(-c2ccc(N(c3ccc(-c4ccccc4)cc3)c3ccccc3-c3ccc4oc5cc(-c6ccccc6)c6ccccc6c5c4c3)cc2)cc1 ; CC1(C)c2ccccc2-c2ccc(N(c3ccc(-c4ccccc4)cc3)c3ccccc3-c3ccc4oc5cc(-c6ccccc6)c6ccccc6c5c4c3)cc21]

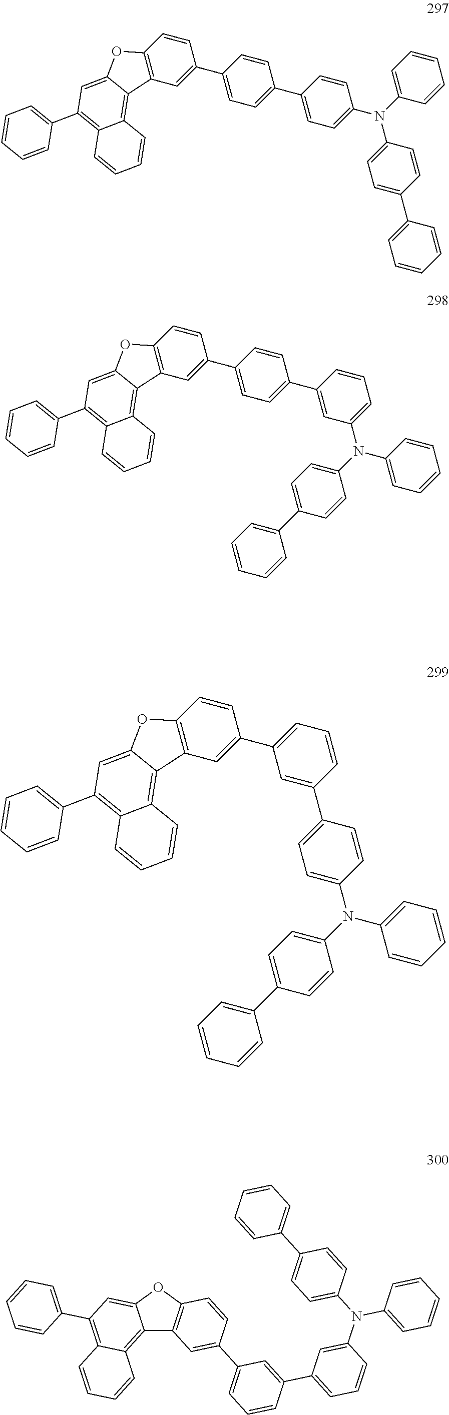 [SMILES: c1ccc(-c2ccc(N(c3ccccc3)c3ccc(-c4ccc(-c5ccc6oc7cc(-c8ccccc8)c8ccccc8c7c6c5)cc4)cc3)cc2)cc1 ; c1ccc(-c2ccc(N(c3ccccc3)c3cccc(-c4ccc(-c5ccc6oc7cc(-c8ccccc8)c8ccccc8c7c6c5)cc4)c3)cc2)cc1 ; c1ccc(-c2ccc(N(c3ccccc3)c3ccc(-c4cccc(-c5ccc6oc7cc(-c8ccccc8)c8ccccc8c7c6c5)c4)cc3)cc2)cc1 ; c1ccc(-c2ccc(N(c3ccccc3)c3cccc(-c4cccc(-c5ccc6oc7cc(-c8ccccc8)c8ccccc8c7c6c5)c4)c3)cc2)cc1]

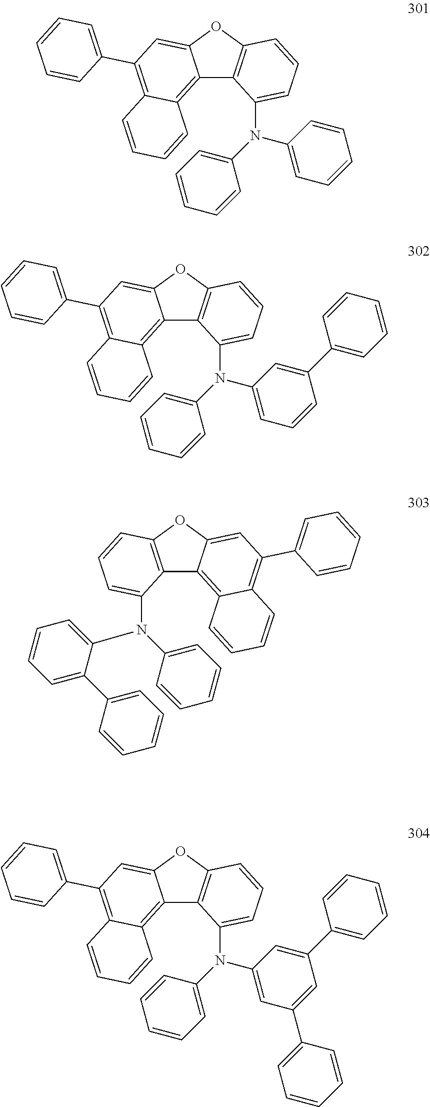 [SMILES: c1ccc(-c2cc3oc4cccc(N(c5ccccc5)c5ccccc5)c4c3c3ccccc23)cc1 ; c1ccc(-c2cccc(N(c3ccccc3)c3cccc4oc5cc(-c6ccccc6)c6ccccc6c5c34)c2)cc1 ; c1ccc(-c2ccccc2N(c2ccccc2)c2cccc3oc4cc(-c5ccccc5)c5ccccc5c4c23)cc1 ; c1ccc(-c2cc(-c3ccccc3)cc(N(c3ccccc3)c3cccc4oc5cc(-c6ccccc6)c6ccccc6c5c34)c2)cc1]

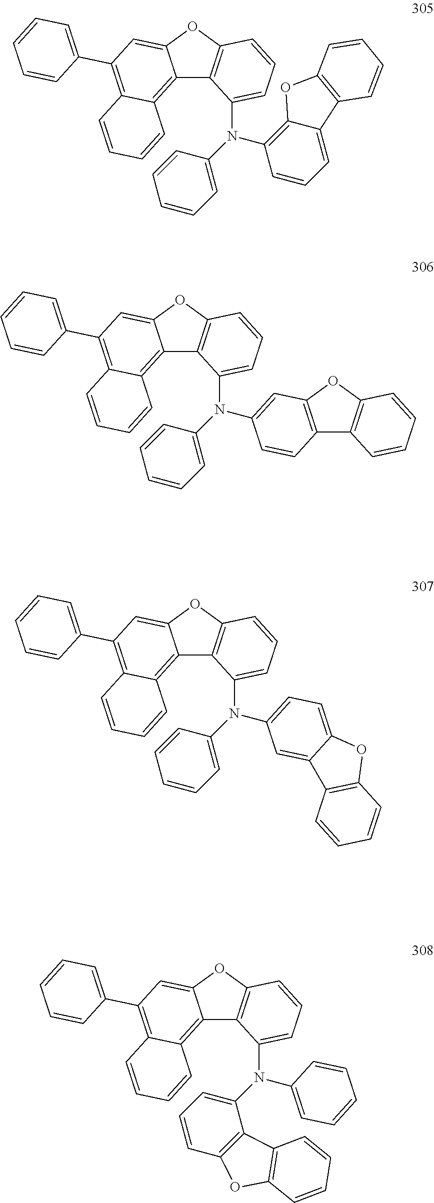 [SMILES: c1ccc(-c2cc3oc4cccc(N(c5ccccc5)c5cccc6c5oc5ccccc56)c4c3c3ccccc23)cc1 ; c1ccc(-c2cc3oc4cccc(N(c5ccccc5)c5ccc6c(c5)oc5ccccc56)c4c3c3ccccc23)cc1 ; c1ccc(-c2cc3oc4cccc(N(c5ccccc5)c5ccc6oc7ccccc7c6c5)c4c3c3ccccc23)cc1 ; c1ccc(-c2cc3oc4cccc(N(c5ccccc5)c5cccc6oc7ccccc7c56)c4c3c3ccccc23)cc1]

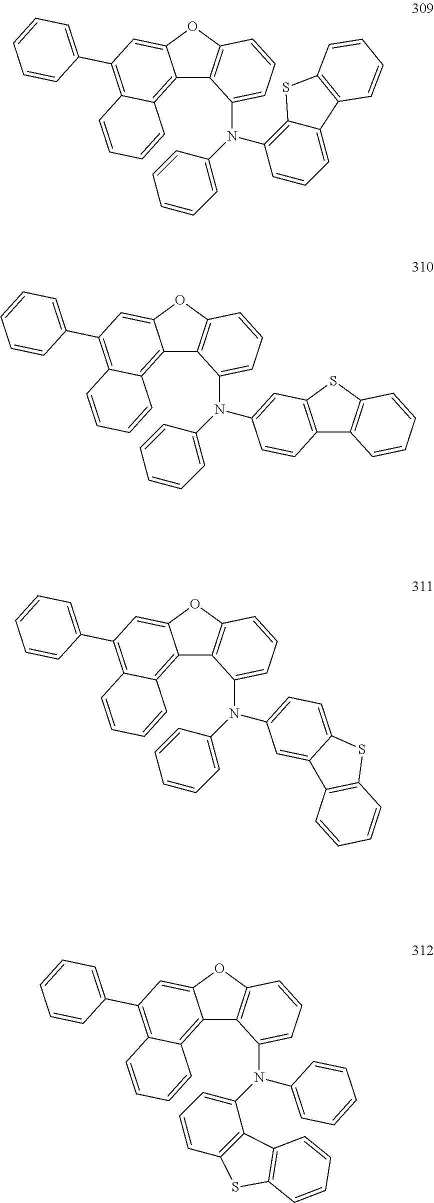 [SMILES: c1ccc(-c2cc3oc4cccc(N(c5ccccc5)c5cccc6c5sc5ccccc56)c4c3c3ccccc23)cc1 ; c1ccc(-c2cc3oc4cccc(N(c5ccccc5)c5ccc6c(c5)sc5ccccc56)c4c3c3ccccc23)cc1 ; c1ccc(-c2cc3oc4cccc(N(c5ccccc5)c5ccc6sc7ccccc7c6c5)c4c3c3ccccc23)cc1 ; c1ccc(-c2cc3oc4cccc(N(c5ccccc5)c5cccc6sc7ccccc7c56)c4c3c3ccccc23)cc1]

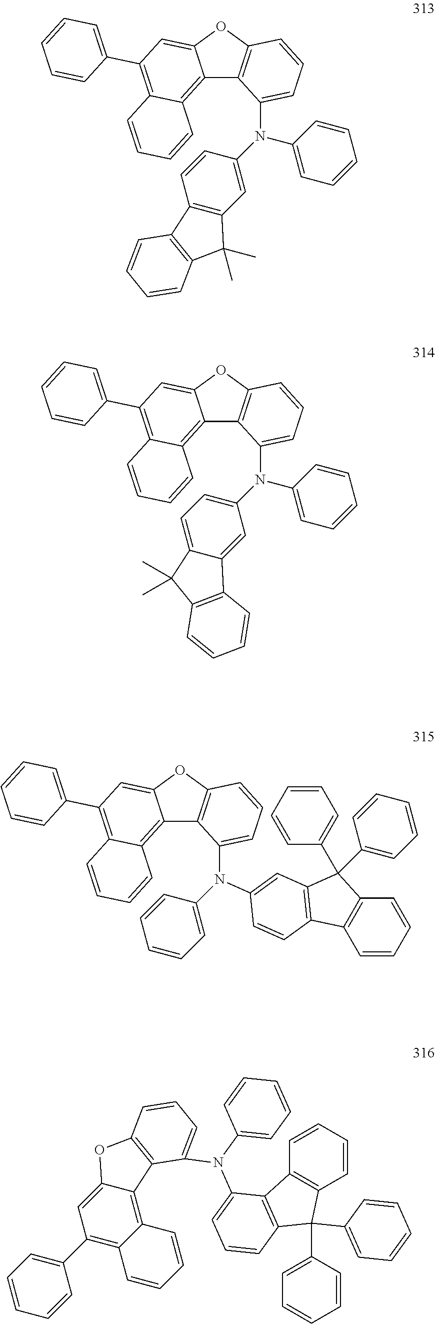 [SMILES: CC1(C)c2ccccc2-c2ccc(N(c3ccccc3)c3cccc4oc5cc(-c6ccccc6)c6ccccc6c5c34)cc21 ; CC1(C)c2ccccc2-c2cc(N(c3ccccc3)c3cccc4oc5cc(-c6ccccc6)c6ccccc6c5c34)ccc21 ; c1ccc(-c2cc3oc4cccc(N(c5ccccc5)c5ccc6c(c5)C(c5ccccc5)(c5ccccc5)c5ccccc5-6)c4c3c3ccccc23)cc1 ; c1ccc(-c2cc3oc4cccc(N(c5ccccc5)c5cccc6c5-c5ccccc5C6(c5ccccc5)c5ccccc5)c4c3c3ccccc23)cc1]

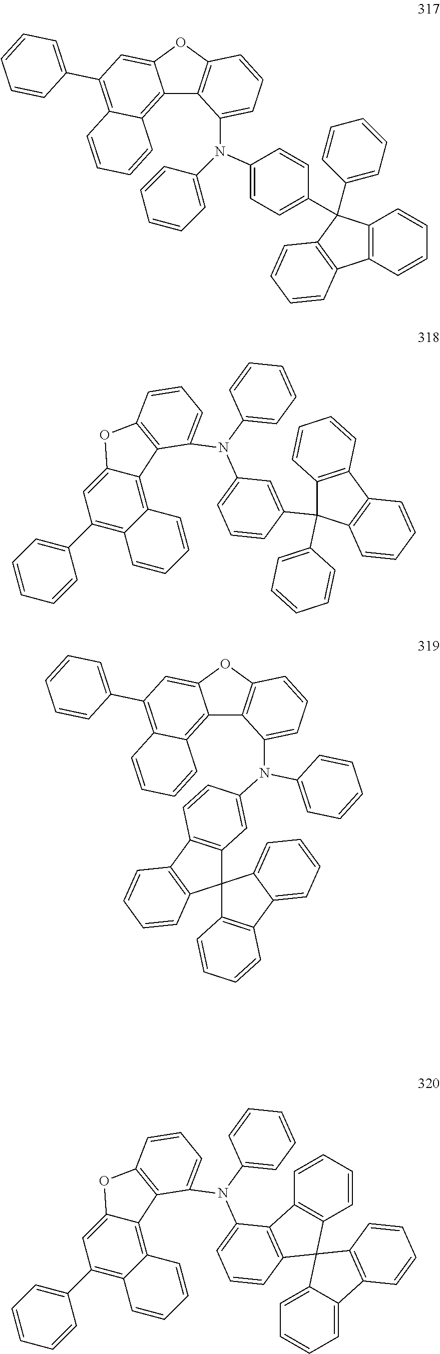 [SMILES: c1ccc(-c2cc3oc4cccc(N(c5ccccc5)c5ccc(C6(c7ccccc7)c7ccccc7-c7ccccc76)cc5)c4c3c3ccccc23)cc1 ; c1ccc(-c2cc3oc4cccc(N(c5ccccc5)c5cccc(C6(c7ccccc7)c7ccccc7-c7ccccc76)c5)c4c3c3ccccc23)cc1 ; c1ccc(-c2cc3oc4cccc(N(c5ccccc5)c5ccc6c(c5)C5(c7ccccc7-c7ccccc75)c5ccccc5-6)c4c3c3ccccc23)cc1 ; c1ccc(-c2cc3oc4cccc(N(c5ccccc5)c5cccc6c5-c5ccccc5C65c6ccccc6-c6ccccc65)c4c3c3ccccc23)cc1]

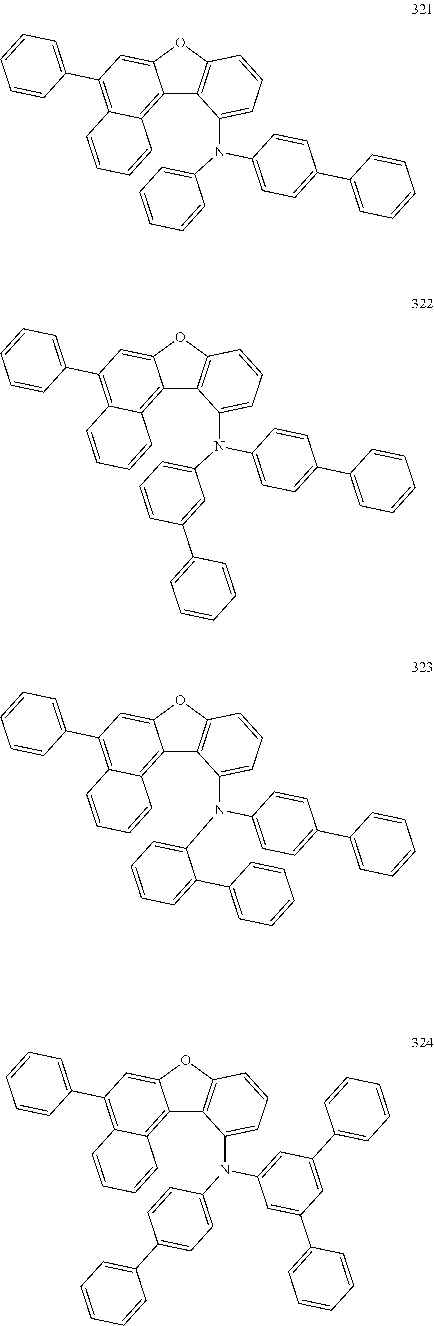 [SMILES: c1ccc(-c2ccc(N(c3ccccc3)c3cccc4oc5cc(-c6ccccc6)c6ccccc6c5c34)cc2)cc1 ; c1ccc(-c2ccc(N(c3cccc(-c4ccccc4)c3)c3cccc4oc5cc(-c6ccccc6)c6ccccc6c5c34)cc2)cc1 ; c1ccc(-c2ccc(N(c3ccccc3-c3ccccc3)c3cccc4oc5cc(-c6ccccc6)c6ccccc6c5c34)cc2)cc1 ; c1ccc(-c2ccc(N(c3cc(-c4ccccc4)cc(-c4ccccc4)c3)c3cccc4oc5cc(-c6ccccc6)c6ccccc6c5c34)cc2)cc1]

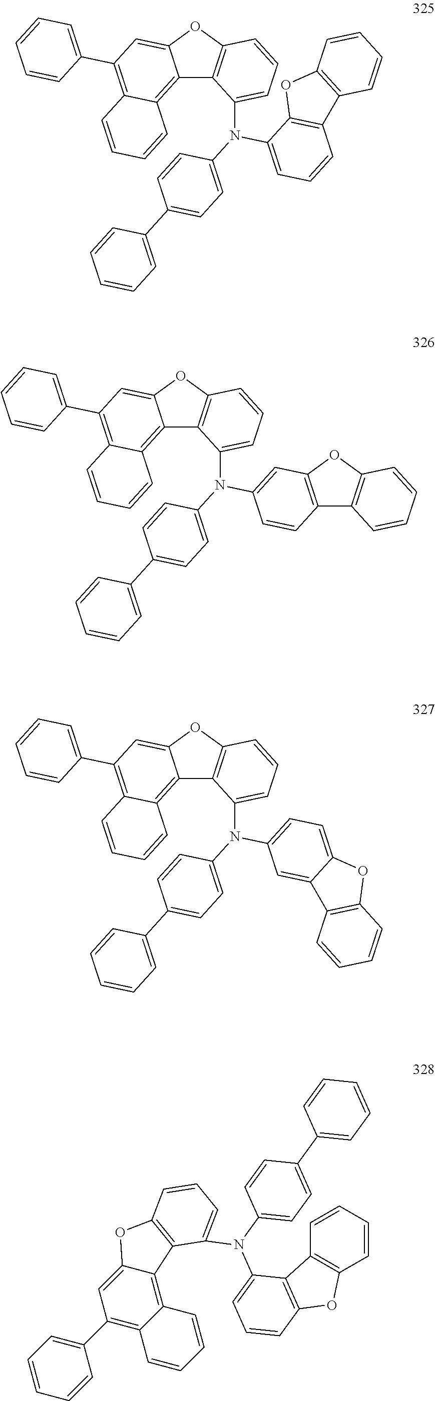 [SMILES: c1ccc(-c2ccc(N(c3cccc4c3oc3ccccc34)c3cccc4oc5cc(-c6ccccc6)c6ccccc6c5c34)cc2)cc1 ; c1ccc(-c2ccc(N(c3ccc4c(c3)oc3ccccc34)c3cccc4oc5cc(-c6ccccc6)c6ccccc6c5c34)cc2)cc1 ; c1ccc(-c2ccc(N(c3ccc4oc5ccccc5c4c3)c3cccc4oc5cc(-c6ccccc6)c6ccccc6c5c34)cc2)cc1 ; c1ccc(-c2ccc(N(c3cccc4oc5ccccc5c34)c3cccc4oc5cc(-c6ccccc6)c6ccccc6c5c34)cc2)cc1]

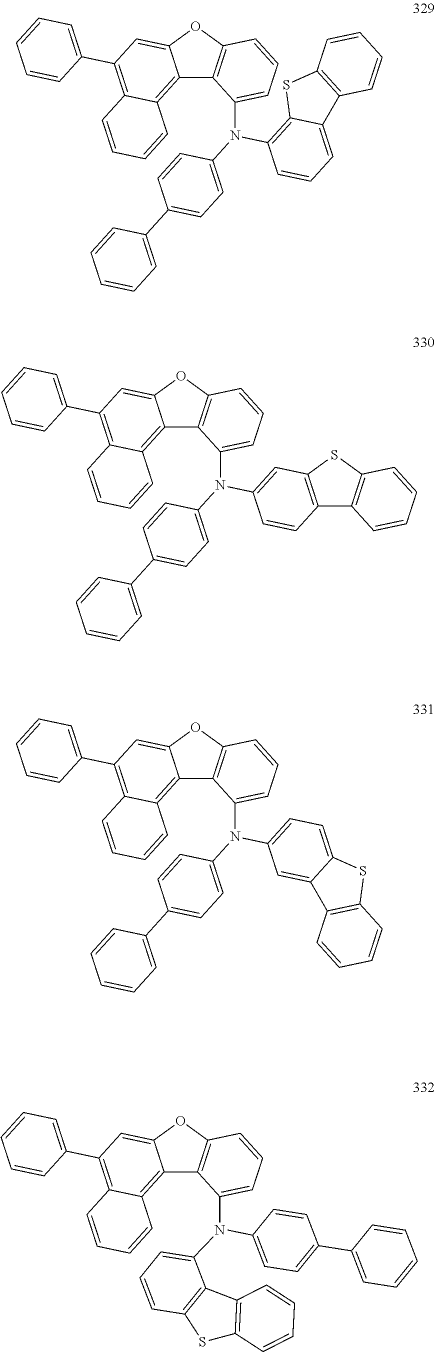 [SMILES: c1ccc(-c2ccc(N(c3cccc4c3sc3ccccc34)c3cccc4oc5cc(-c6ccccc6)c6ccccc6c5c34)cc2)cc1 ; c1ccc(-c2ccc(N(c3ccc4c(c3)sc3ccccc34)c3cccc4oc5cc(-c6ccccc6)c6ccccc6c5c34)cc2)cc1 ; c1ccc(-c2ccc(N(c3ccc4sc5ccccc5c4c3)c3cccc4oc5cc(-c6ccccc6)c6ccccc6c5c34)cc2)cc1 ; c1ccc(-c2ccc(N(c3cccc4sc5ccccc5c34)c3cccc4oc5cc(-c6ccccc6)c6ccccc6c5c34)cc2)cc1]

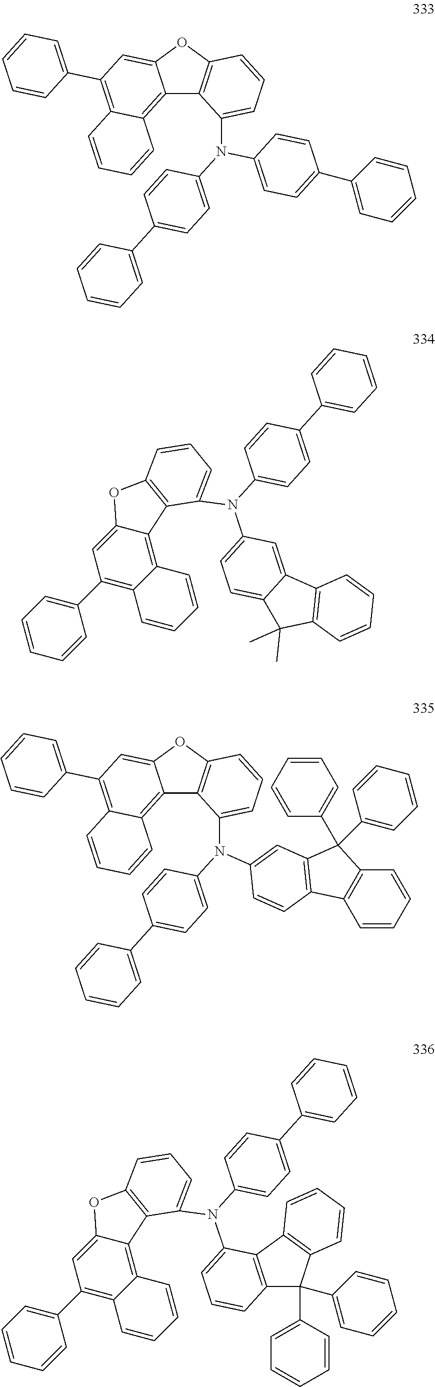 [SMILES: c1ccc(-c2ccc(N(c3ccc(-c4ccccc4)cc3)c3cccc4oc5cc(-c6ccccc6)c6ccccc6c5c34)cc2)cc1 ; CC1(C)c2ccccc2-c2cc(N(c3ccc(-c4ccccc4)cc3)c3cccc4oc5cc(-c6ccccc6)c6ccccc6c5c34)ccc21 ; c1ccc(-c2ccc(N(c3ccc4c(c3)C(c3ccccc3)(c3ccccc3)c3ccccc3-4)c3cccc4oc5cc(-c6ccccc6)c6ccccc6c5c34)cc2)cc1 ; c1ccc(-c2ccc(N(c3cccc4c3-c3ccccc3C4(c3ccccc3)c3ccccc3)c3cccc4oc5cc(-c6ccccc6)c6ccccc6c5c34)cc2)cc1]

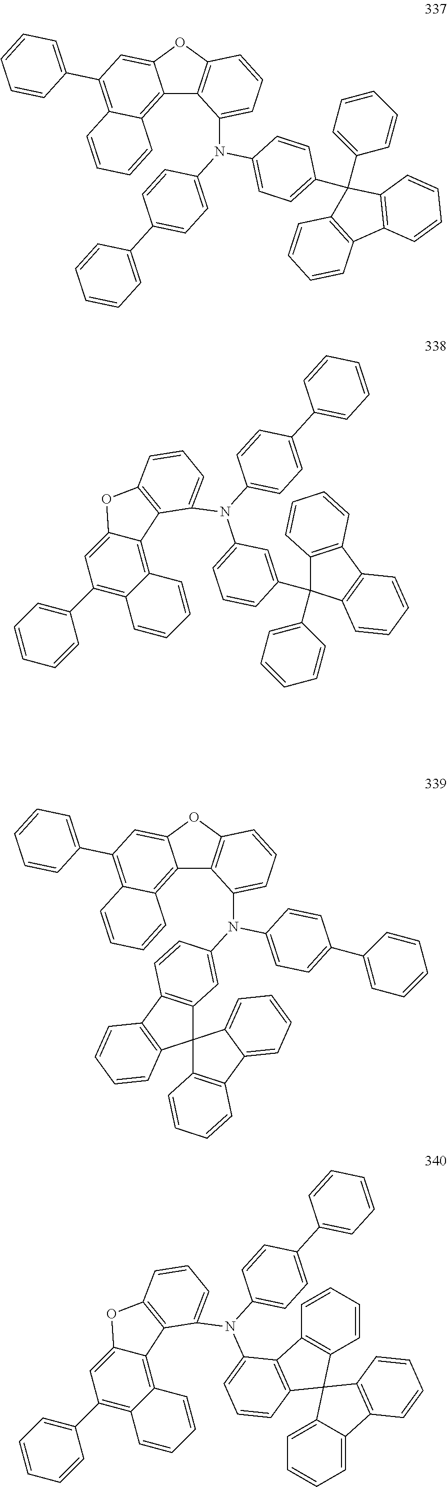 [SMILES: c1ccc(-c2ccc(N(c3ccc(C4(c5ccccc5)c5ccccc5-c5ccccc54)cc3)c3cccc4oc5cc(-c6ccccc6)c6ccccc6c5c34)cc2)cc1 ; c1ccc(-c2ccc(N(c3cccc(C4(c5ccccc5)c5ccccc5-c5ccccc54)c3)c3cccc4oc5cc(-c6ccccc6)c6ccccc6c5c34)cc2)cc1 ; c1ccc(-c2ccc(N(c3ccc4c(c3)C3(c5ccccc5-c5ccccc53)c3ccccc3-4)c3cccc4oc5cc(-c6ccccc6)c6ccccc6c5c34)cc2)cc1 ; c1ccc(-c2ccc(N(c3cccc4c3-c3ccccc3C43c4ccccc4-c4ccccc43)c3cccc4oc5cc(-c6ccccc6)c6ccccc6c5c34)cc2)cc1]

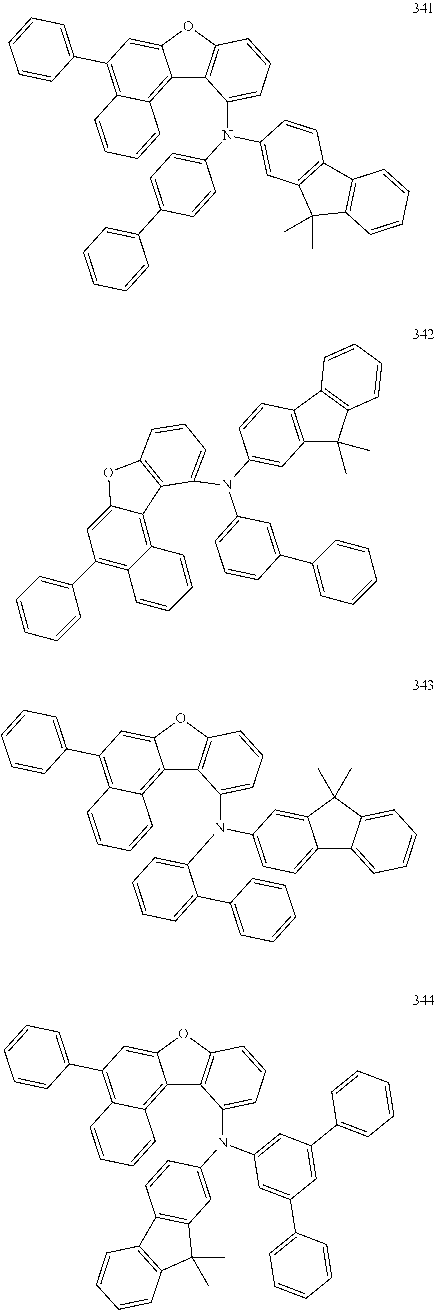 [SMILES: CC1(C)c2ccccc2-c2ccc(N(c3ccc(-c4ccccc4)cc3)c3cccc4oc5cc(-c6ccccc6)c6ccccc6c5c34)cc21 ; CC1(C)c2ccccc2-c2ccc(N(c3cccc(-c4ccccc4)c3)c3cccc4oc5cc(-c6ccccc6)c6ccccc6c5c34)cc21 ; CC1(C)c2ccccc2-c2ccc(N(c3ccccc3-c3ccccc3)c3cccc4oc5cc(-c6ccccc6)c6ccccc6c5c34)cc21 ; CC1(C)c2ccccc2-c2ccc(N(c3cc(-c4ccccc4)cc(-c4ccccc4)c3)c3cccc4oc5cc(-c6ccccc6)c6ccccc6c5c34)cc21]

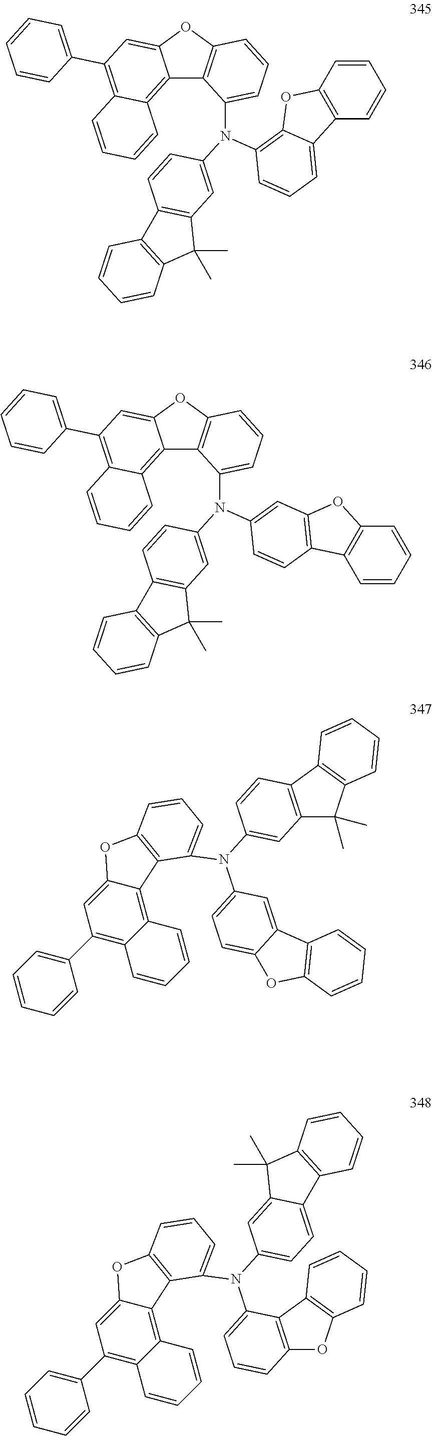 [SMILES: CC1(C)c2ccccc2-c2ccc(N(c3cccc4c3oc3ccccc34)c3cccc4oc5cc(-c6ccccc6)c6ccccc6c5c34)cc21 ; CC1(C)c2ccccc2-c2ccc(N(c3ccc4c(c3)oc3ccccc34)c3cccc4oc5cc(-c6ccccc6)c6ccccc6c5c34)cc21 ; CC1(C)c2ccccc2-c2ccc(N(c3ccc4oc5ccccc5c4c3)c3cccc4oc5cc(-c6ccccc6)c6ccccc6c5c34)cc21 ; CC1(C)c2ccccc2-c2ccc(N(c3cccc4oc5ccccc5c34)c3cccc4oc5cc(-c6ccccc6)c6ccccc6c5c34)cc21]

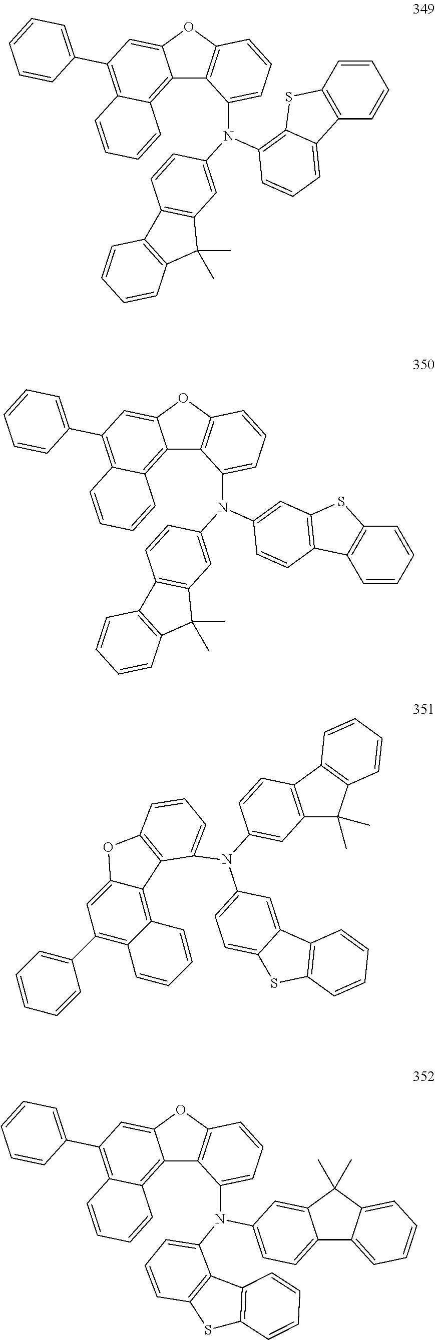 [SMILES: CC1(C)c2ccccc2-c2ccc(N(c3cccc4c3sc3ccccc34)c3cccc4oc5cc(-c6ccccc6)c6ccccc6c5c34)cc21 ; CC1(C)c2ccccc2-c2ccc(N(c3ccc4c(c3)sc3ccccc34)c3cccc4oc5cc(-c6ccccc6)c6ccccc6c5c34)cc21 ; CC1(C)c2ccccc2-c2ccc(N(c3ccc4sc5ccccc5c4c3)c3cccc4oc5cc(-c6ccccc6)c6ccccc6c5c34)cc21 ; CC1(C)c2ccccc2-c2ccc(N(c3cccc4sc5ccccc5c34)c3cccc4oc5cc(-c6ccccc6)c6ccccc6c5c34)cc21]

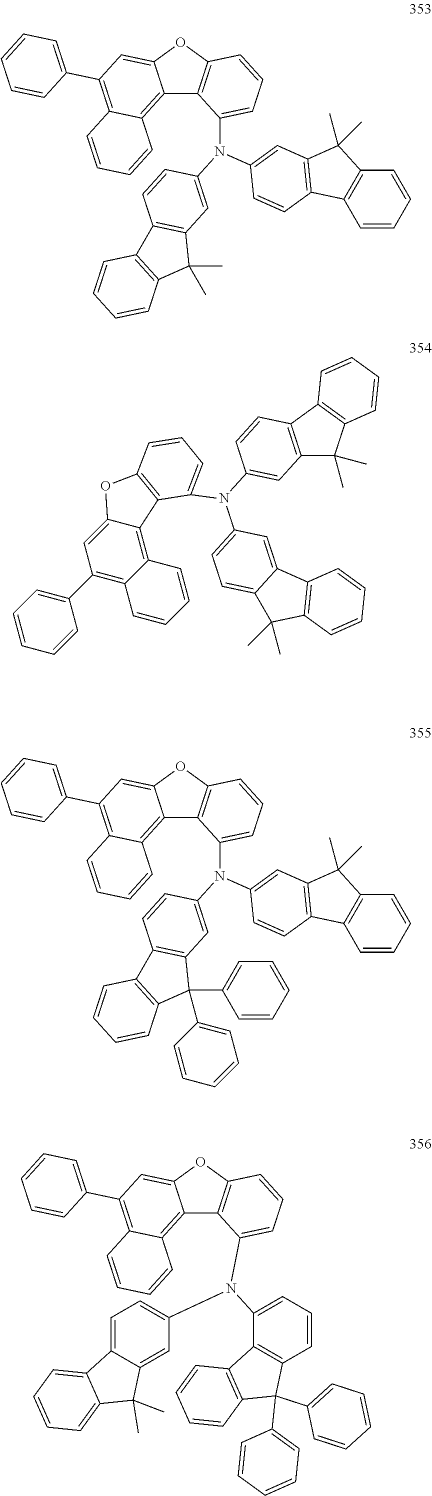 [SMILES: CC1(C)c2ccccc2-c2ccc(N(c3ccc4c(c3)C(C)(C)c3ccccc3-4)c3cccc4oc5cc(-c6ccccc6)c6ccccc6c5c34)cc21 ; CC1(C)c2ccccc2-c2cc(N(c3ccc4c(c3)C(C)(C)c3ccccc3-4)c3cccc4oc5cc(-c6ccccc6)c6ccccc6c5c34)ccc21 ; CC1(C)c2ccccc2-c2ccc(N(c3ccc4c(c3)C(c3ccccc3)(c3ccccc3)c3ccccc3-4)c3cccc4oc5cc(-c6ccccc6)c6ccccc6c5c34)cc21 ; CC1(C)c2ccccc2-c2ccc(N(c3cccc4c3-c3ccccc3C4(c3ccccc3)c3ccccc3)c3cccc4oc5cc(-c6ccccc6)c6ccccc6c5c34)cc21]

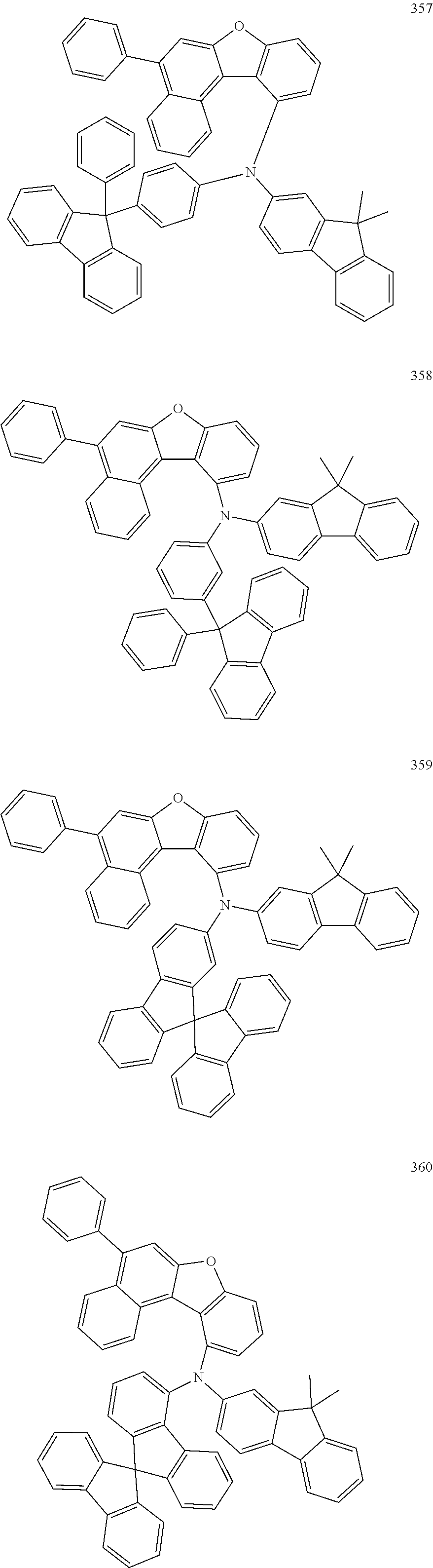 [SMILES: CC1(C)c2ccccc2-c2ccc(N(c3ccc(C4(c5ccccc5)c5ccccc5-c5ccccc54)cc3)c3cccc4oc5cc(-c6ccccc6)c6ccccc6c5c34)cc21 ; CC1(C)c2ccccc2-c2ccc(N(c3cccc(C4(c5ccccc5)c5ccccc5-c5ccccc54)c3)c3cccc4oc5cc(-c6ccccc6)c6ccccc6c5c34)cc21 ; CC1(C)c2ccccc2-c2ccc(N(c3ccc4c(c3)C3(c5ccccc5-c5ccccc53)c3ccccc3-4)c3cccc4oc5cc(-c6ccccc6)c6ccccc6c5c34)cc21 ; CC1(C)c2ccccc2-c2ccc(N(c3cccc4c3-c3ccccc3C43c4ccccc4-c4ccccc43)c3cccc4oc5cc(-c6ccccc6)c6ccccc6c5c34)cc21]

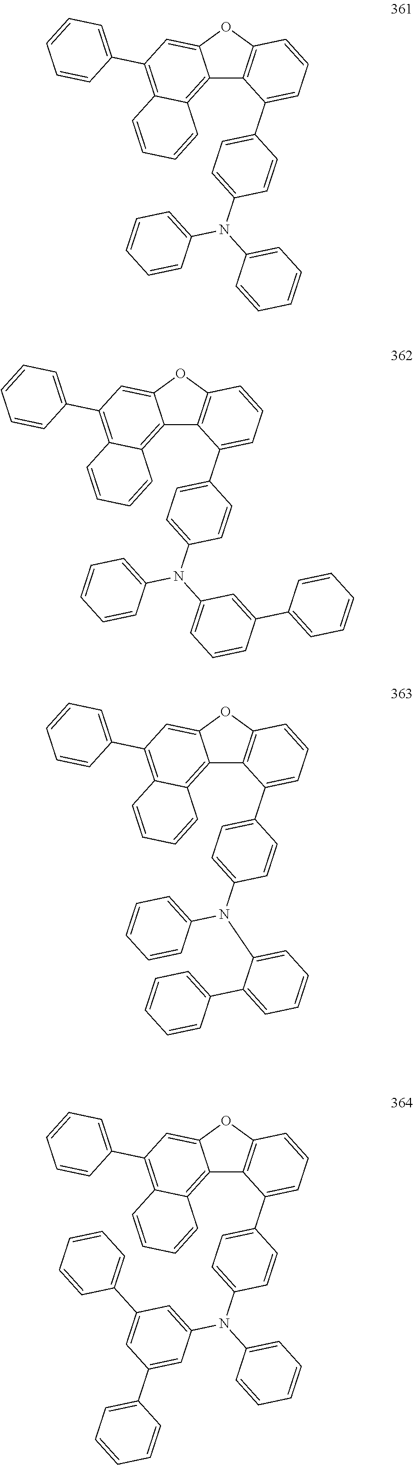 [SMILES: c1ccc(-c2cc3oc4cccc(-c5ccc(N(c6ccccc6)c6ccccc6)cc5)c4c3c3ccccc23)cc1 ; c1ccc(-c2cccc(N(c3ccccc3)c3ccc(-c4cccc5oc6cc(-c7ccccc7)c7ccccc7c6c45)cc3)c2)cc1 ; c1ccc(-c2ccccc2N(c2ccccc2)c2ccc(-c3cccc4oc5cc(-c6ccccc6)c6ccccc6c5c34)cc2)cc1 ; c1ccc(-c2cc(-c3ccccc3)cc(N(c3ccccc3)c3ccc(-c4cccc5oc6cc(-c7ccccc7)c7ccccc7c6c45)cc3)c2)cc1]

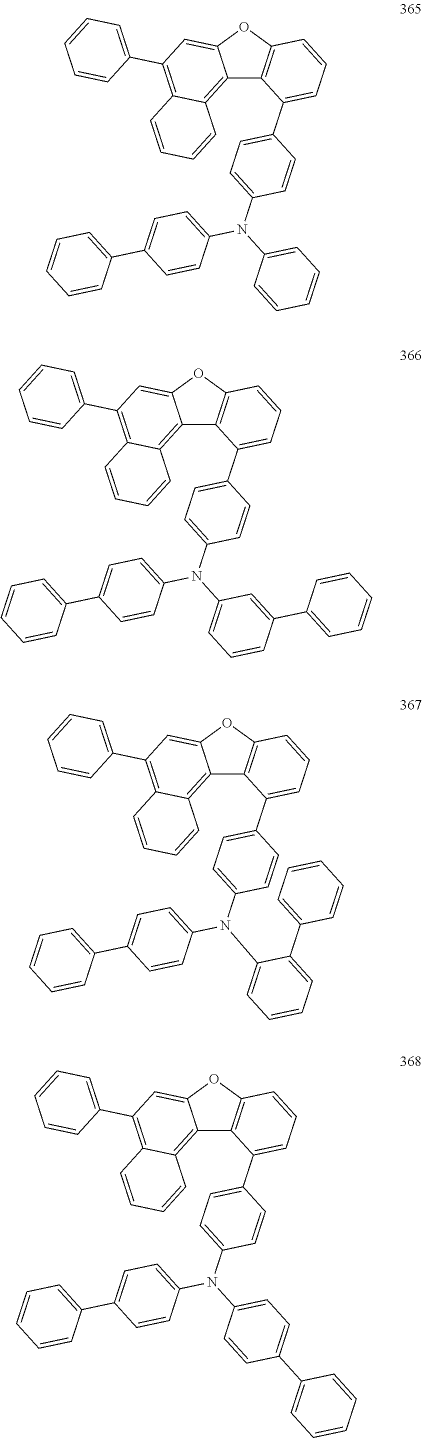 [SMILES: c1ccc(-c2ccc(N(c3ccccc3)c3ccc(-c4cccc5oc6cc(-c7ccccc7)c7ccccc7c6c45)cc3)cc2)cc1 ; c1ccc(-c2ccc(N(c3ccc(-c4cccc5oc6cc(-c7ccccc7)c7ccccc7c6c45)cc3)c3cccc(-c4ccccc4)c3)cc2)cc1 ; c1ccc(-c2ccc(N(c3ccc(-c4cccc5oc6cc(-c7ccccc7)c7ccccc7c6c45)cc3)c3ccccc3-c3ccccc3)cc2)cc1 ; c1ccc(-c2ccc(N(c3ccc(-c4ccccc4)cc3)c3ccc(-c4cccc5oc6cc(-c7ccccc7)c7ccccc7c6c45)cc3)cc2)cc1]

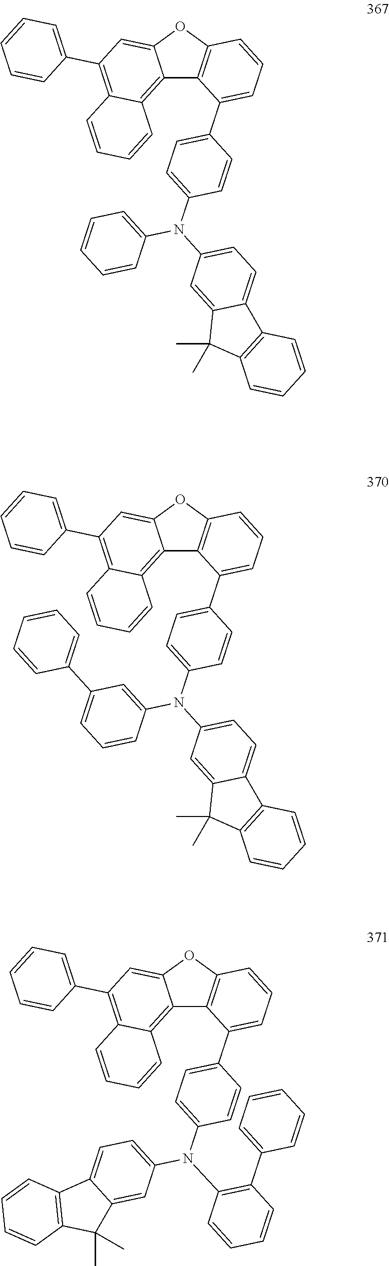 [SMILES: CC1(C)c2ccccc2-c2ccc(N(c3ccccc3)c3ccc(-c4cccc5oc6cc(-c7ccccc7)c7ccccc7c6c45)cc3)cc21 ; CC1(C)c2ccccc2-c2ccc(N(c3ccc(-c4cccc5oc6cc(-c7ccccc7)c7ccccc7c6c45)cc3)c3cccc(-c4ccccc4)c3)cc21 ; CC1(C)c2ccccc2-c2ccc(N(c3ccc(-c4cccc5oc6cc(-c7ccccc7)c7ccccc7c6c45)cc3)c3ccccc3-c3ccccc3)cc21]

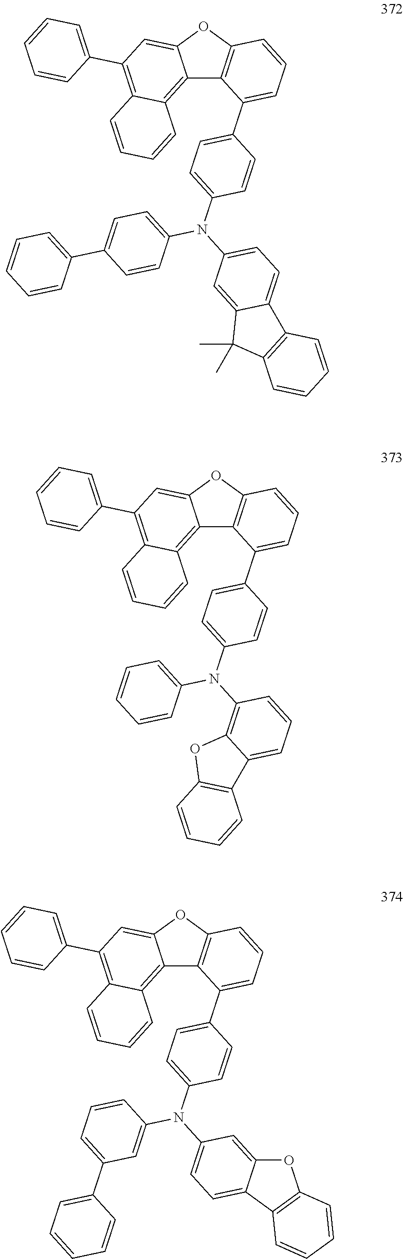 [SMILES: CC1(C)c2ccccc2-c2ccc(N(c3ccc(-c4ccccc4)cc3)c3ccc(-c4cccc5oc6cc(-c7ccccc7)c7ccccc7c6c45)cc3)cc21 ; c1ccc(-c2cc3oc4cccc(-c5ccc(N(c6ccccc6)c6cccc7c6oc6ccccc67)cc5)c4c3c3ccccc23)cc1 ; c1ccc(-c2cccc(N(c3ccc(-c4cccc5oc6cc(-c7ccccc7)c7ccccc7c6c45)cc3)c3ccc4c(c3)oc3ccccc34)c2)cc1]

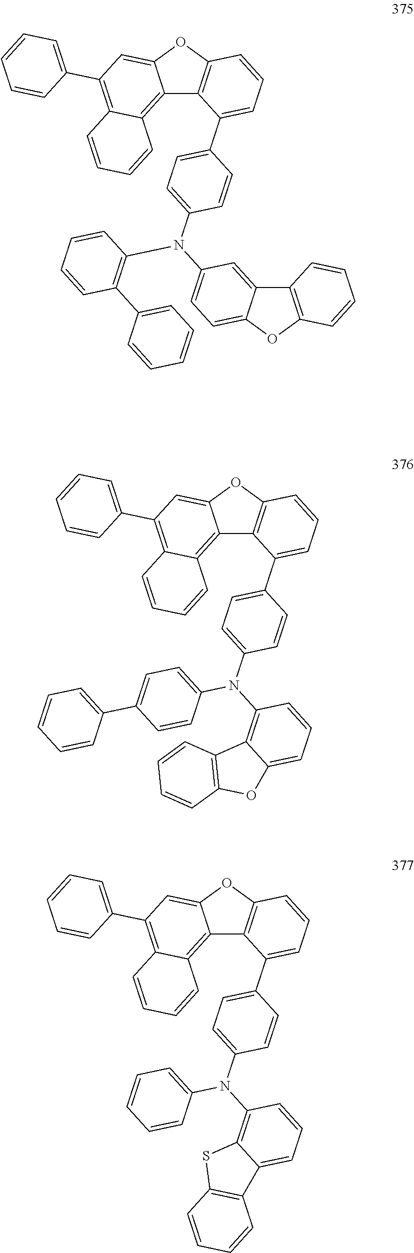 [SMILES: c1ccc(-c2ccccc2N(c2ccc(-c3cccc4oc5cc(-c6ccccc6)c6ccccc6c5c34)cc2)c2ccc3oc4ccccc4c3c2)cc1 ; c1ccc(-c2ccc(N(c3ccc(-c4cccc5oc6cc(-c7ccccc7)c7ccccc7c6c45)cc3)c3cccc4oc5ccccc5c34)cc2)cc1 ; c1ccc(-c2cc3oc4cccc(-c5ccc(N(c6ccccc6)c6cccc7c6sc6ccccc67)cc5)c4c3c3ccccc23)cc1]

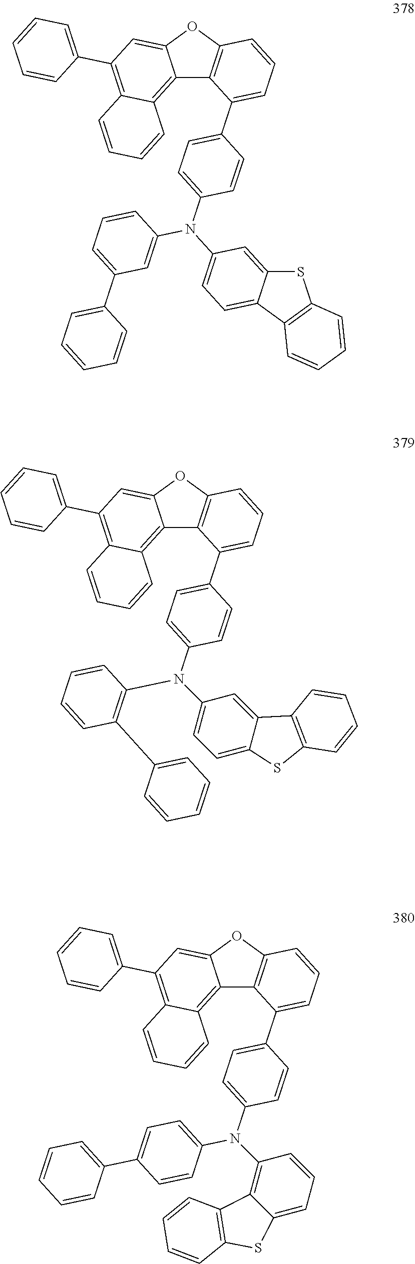 [SMILES: c1ccc(-c2cccc(N(c3ccc(-c4cccc5oc6cc(-c7ccccc7)c7ccccc7c6c45)cc3)c3ccc4c(c3)sc3ccccc34)c2)cc1 ; c1ccc(-c2ccccc2N(c2ccc(-c3cccc4oc5cc(-c6ccccc6)c6ccccc6c5c34)cc2)c2ccc3sc4ccccc4c3c2)cc1 ; c1ccc(-c2ccc(N(c3ccc(-c4cccc5oc6cc(-c7ccccc7)c7ccccc7c6c45)cc3)c3cccc4sc5ccccc5c34)cc2)cc1]

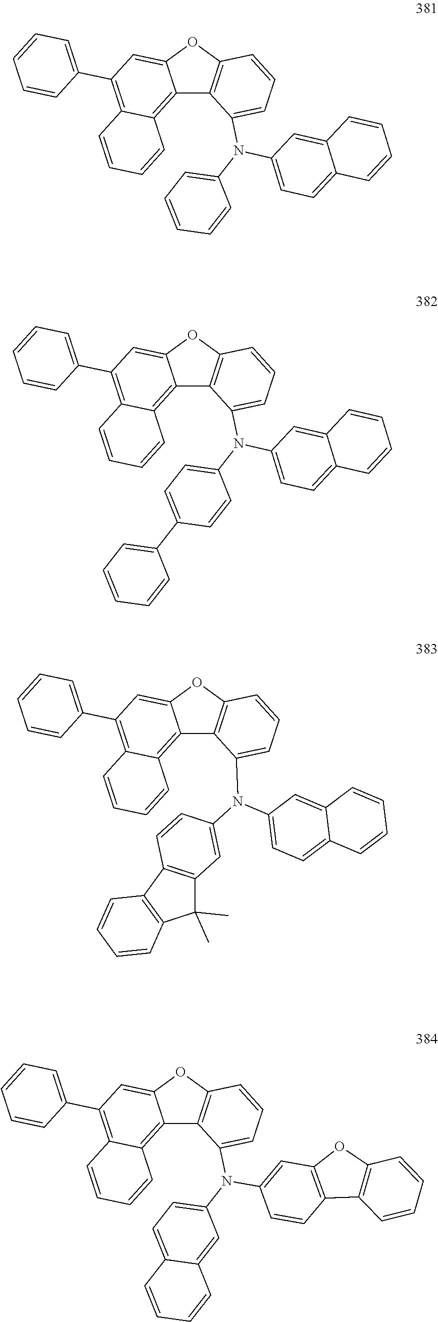 [SMILES: c1ccc(-c2cc3oc4cccc(N(c5ccccc5)c5ccc6ccccc6c5)c4c3c3ccccc23)cc1 ; c1ccc(-c2ccc(N(c3ccc4ccccc4c3)c3cccc4oc5cc(-c6ccccc6)c6ccccc6c5c34)cc2)cc1 ; CC1(C)c2ccccc2-c2ccc(N(c3ccc4ccccc4c3)c3cccc4oc5cc(-c6ccccc6)c6ccccc6c5c34)cc21 ; c1ccc(-c2cc3oc4cccc(N(c5ccc6ccccc6c5)c5ccc6c(c5)oc5ccccc56)c4c3c3ccccc23)cc1]

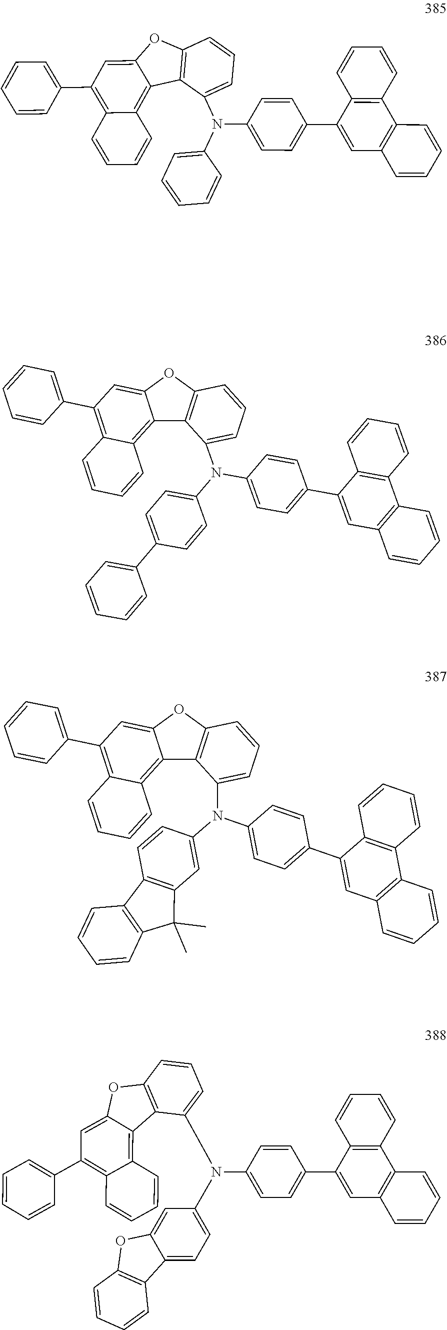 [SMILES: c1ccc(-c2cc3oc4cccc(N(c5ccccc5)c5ccc(-c6cc7ccccc7c7ccccc67)cc5)c4c3c3ccccc23)cc1 ; c1ccc(-c2ccc(N(c3ccc(-c4cc5ccccc5c5ccccc45)cc3)c3cccc4oc5cc(-c6ccccc6)c6ccccc6c5c34)cc2)cc1 ; CC1(C)c2ccccc2-c2ccc(N(c3ccc(-c4cc5ccccc5c5ccccc45)cc3)c3cccc4oc5cc(-c6ccccc6)c6ccccc6c5c34)cc21 ; c1ccc(-c2cc3oc4cccc(N(c5ccc(-c6cc7ccccc7c7ccccc67)cc5)c5ccc6c(c5)oc5ccccc56)c4c3c3ccccc23)cc1]

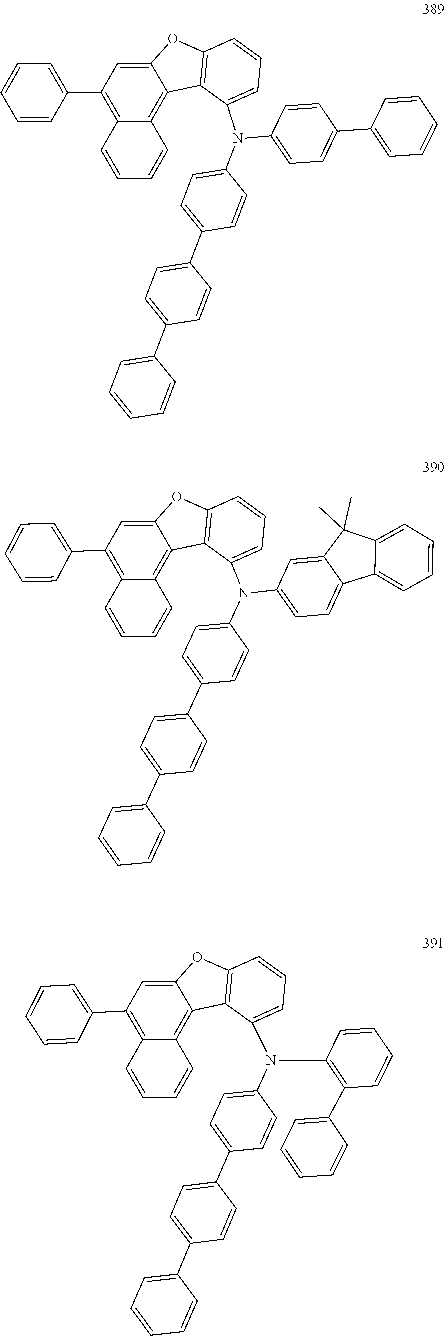 [SMILES: c1ccc(-c2ccc(-c3ccc(N(c4ccc(-c5ccccc5)cc4)c4cccc5oc6cc(-c7ccccc7)c7ccccc7c6c45)cc3)cc2)cc1 ; CC1(C)c2ccccc2-c2ccc(N(c3ccc(-c4ccc(-c5ccccc5)cc4)cc3)c3cccc4oc5cc(-c6ccccc6)c6ccccc6c5c34)cc21 ; c1ccc(-c2ccc(-c3ccc(N(c4ccccc4-c4ccccc4)c4cccc5oc6cc(-c7ccccc7)c7ccccc7c6c45)cc3)cc2)cc1]

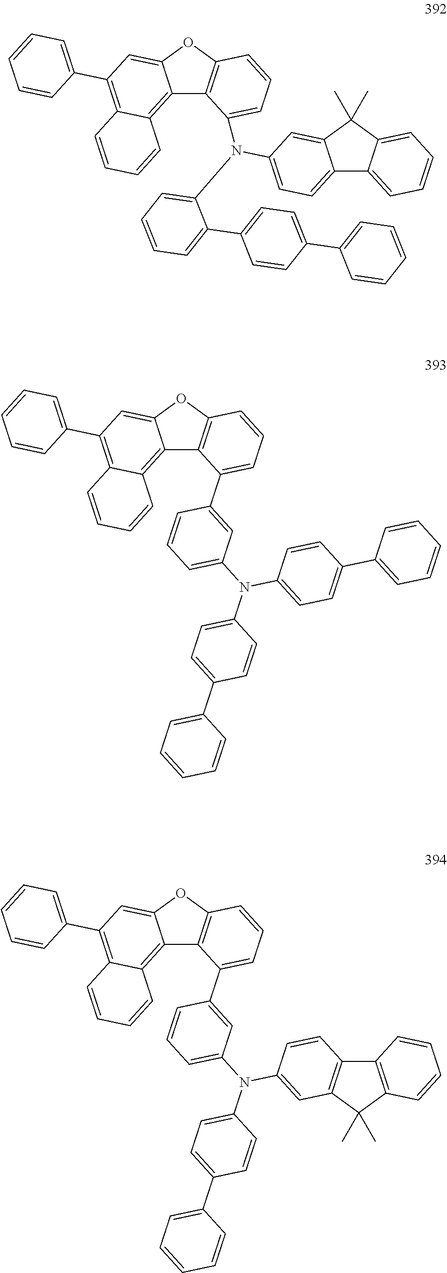 [SMILES: CC1(C)c2ccccc2-c2ccc(N(c3ccccc3-c3ccc(-c4ccccc4)cc3)c3cccc4oc5cc(-c6ccccc6)c6ccccc6c5c34)cc21 ; c1ccc(-c2ccc(N(c3ccc(-c4ccccc4)cc3)c3cccc(-c4cccc5oc6cc(-c7ccccc7)c7ccccc7c6c45)c3)cc2)cc1 ; CC1(C)c2ccccc2-c2ccc(N(c3ccc(-c4ccccc4)cc3)c3cccc(-c4cccc5oc6cc(-c7ccccc7)c7ccccc7c6c45)c3)cc21]

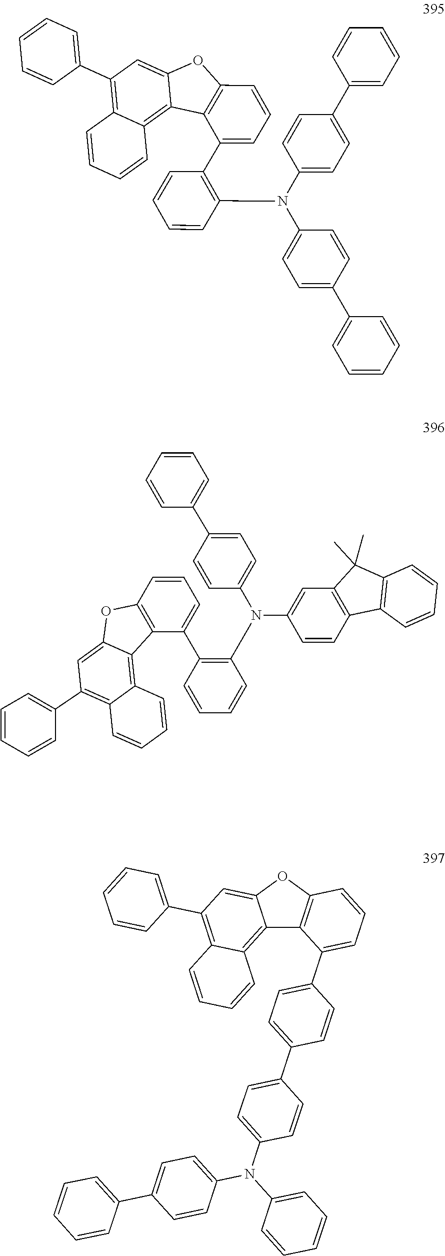 [SMILES: c1ccc(-c2ccc(N(c3ccc(-c4ccccc4)cc3)c3ccccc3-c3cccc4oc5cc(-c6ccccc6)c6ccccc6c5c34)cc2)cc1 ; CC1(C)c2ccccc2-c2ccc(N(c3ccc(-c4ccccc4)cc3)c3ccccc3-c3cccc4oc5cc(-c6ccccc6)c6ccccc6c5c34)cc21 ; c1ccc(-c2ccc(N(c3ccccc3)c3ccc(-c4ccc(-c5cccc6oc7cc(-c8ccccc8)c8ccccc8c7c56)cc4)cc3)cc2)cc1]

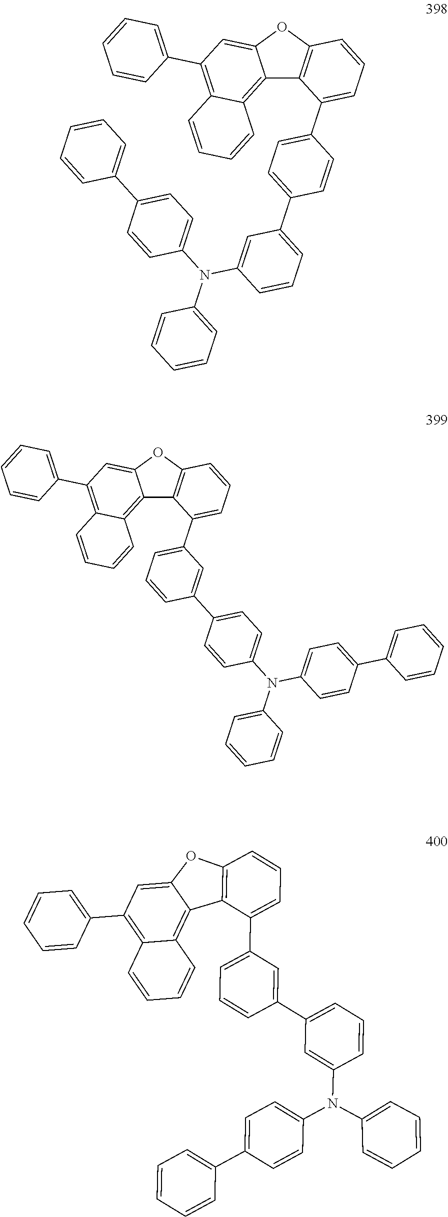 [SMILES: c1ccc(-c2ccc(N(c3ccccc3)c3cccc(-c4ccc(-c5cccc6oc7cc(-c8ccccc8)c8ccccc8c7c56)cc4)c3)cc2)cc1 ; c1ccc(-c2ccc(N(c3ccccc3)c3ccc(-c4cccc(-c5cccc6oc7cc(-c8ccccc8)c8ccccc8c7c56)c4)cc3)cc2)cc1 ; c1ccc(-c2ccc(N(c3ccccc3)c3cccc(-c4cccc(-c5cccc6oc7cc(-c8ccccc8)c8ccccc8c7c56)c4)c3)cc2)cc1]

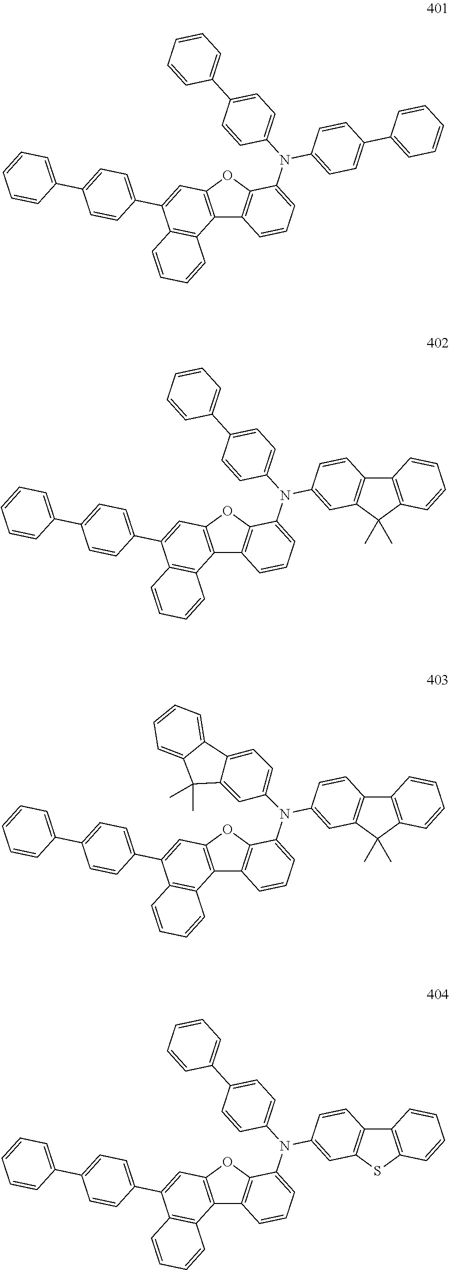 [SMILES: c1ccc(-c2ccc(-c3cc4oc5c(N(c6ccc(-c7ccccc7)cc6)c6ccc(-c7ccccc7)cc6)cccc5c4c4ccccc34)cc2)cc1 ; CC1(C)c2ccccc2-c2ccc(N(c3ccc(-c4ccccc4)cc3)c3cccc4c3oc3cc(-c5ccc(-c6ccccc6)cc5)c5ccccc5c34)cc21 ; CC1(C)c2ccccc2-c2ccc(N(c3ccc4c(c3)C(C)(C)c3ccccc3-4)c3cccc4c3oc3cc(-c5ccc(-c6ccccc6)cc5)c5ccccc5c34)cc21 ; c1ccc(-c2ccc(-c3cc4oc5c(N(c6ccc(-c7ccccc7)cc6)c6ccc7c(c6)sc6ccccc67)cccc5c4c4ccccc34)cc2)cc1]

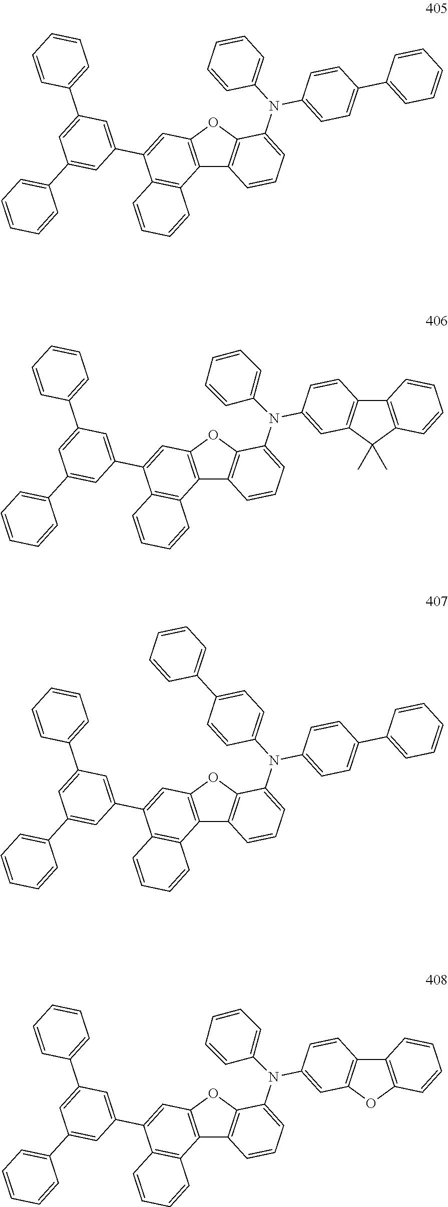 [SMILES: c1ccc(-c2ccc(N(c3ccccc3)c3cccc4c3oc3cc(-c5cc(-c6ccccc6)cc(-c6ccccc6)c5)c5ccccc5c34)cc2)cc1 ; CC1(C)c2ccccc2-c2ccc(N(c3ccccc3)c3cccc4c3oc3cc(-c5cc(-c6ccccc6)cc(-c6ccccc6)c5)c5ccccc5c34)cc21 ; c1ccc(-c2ccc(N(c3ccc(-c4ccccc4)cc3)c3cccc4c3oc3cc(-c5cc(-c6ccccc6)cc(-c6ccccc6)c5)c5ccccc5c34)cc2)cc1 ; c1ccc(-c2cc(-c3ccccc3)cc(-c3cc4oc5c(N(c6ccccc6)c6ccc7c(c6)oc6ccccc67)cccc5c4c4ccccc34)c2)cc1]

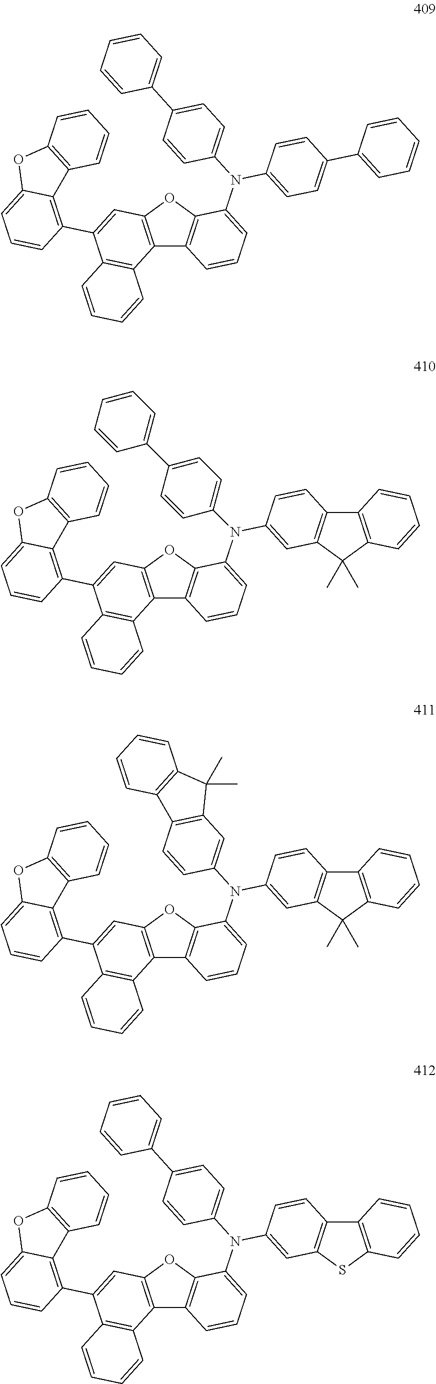 [SMILES: c1ccc(-c2ccc(N(c3ccc(-c4ccccc4)cc3)c3cccc4c3oc3cc(-c5cccc6oc7ccccc7c56)c5ccccc5c34)cc2)cc1 ; CC1(C)c2ccccc2-c2ccc(N(c3ccc(-c4ccccc4)cc3)c3cccc4c3oc3cc(-c5cccc6oc7ccccc7c56)c5ccccc5c34)cc21 ; CC1(C)c2ccccc2-c2ccc(N(c3ccc4c(c3)C(C)(C)c3ccccc3-4)c3cccc4c3oc3cc(-c5cccc6oc7ccccc7c56)c5ccccc5c34)cc21 ; c1ccc(-c2ccc(N(c3ccc4c(c3)sc3ccccc34)c3cccc4c3oc3cc(-c5cccc6oc7ccccc7c56)c5ccccc5c34)cc2)cc1]

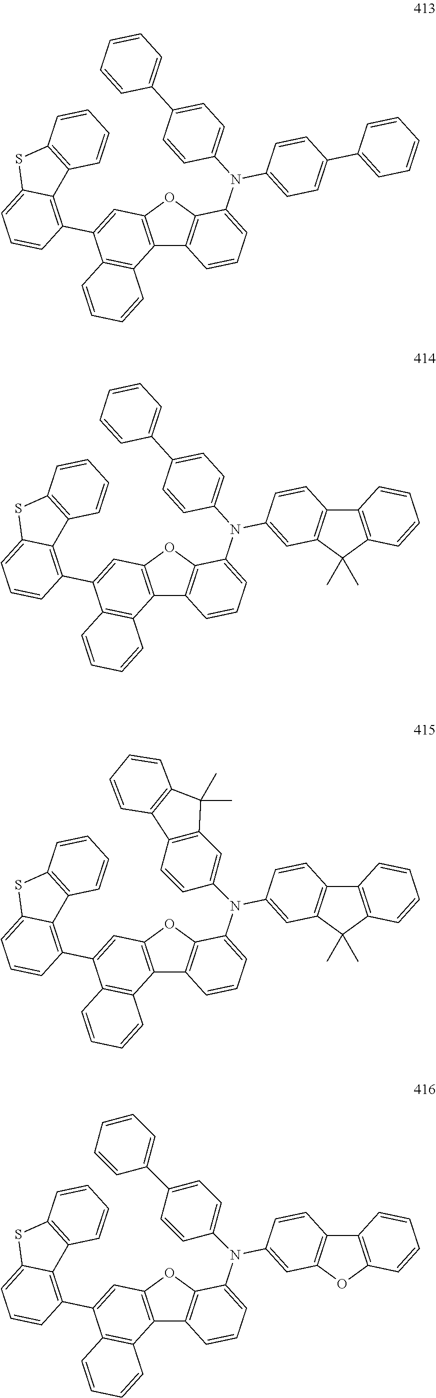 [SMILES: c1ccc(-c2ccc(N(c3ccc(-c4ccccc4)cc3)c3cccc4c3oc3cc(-c5cccc6sc7ccccc7c56)c5ccccc5c34)cc2)cc1 ; CC1(C)c2ccccc2-c2ccc(N(c3ccc(-c4ccccc4)cc3)c3cccc4c3oc3cc(-c5cccc6sc7ccccc7c56)c5ccccc5c34)cc21 ; CC1(C)c2ccccc2-c2ccc(N(c3ccc4c(c3)C(C)(C)c3ccccc3-4)c3cccc4c3oc3cc(-c5cccc6sc7ccccc7c56)c5ccccc5c34)cc21 ; c1ccc(-c2ccc(N(c3ccc4c(c3)oc3ccccc34)c3cccc4c3oc3cc(-c5cccc6sc7ccccc7c56)c5ccccc5c34)cc2)cc1]

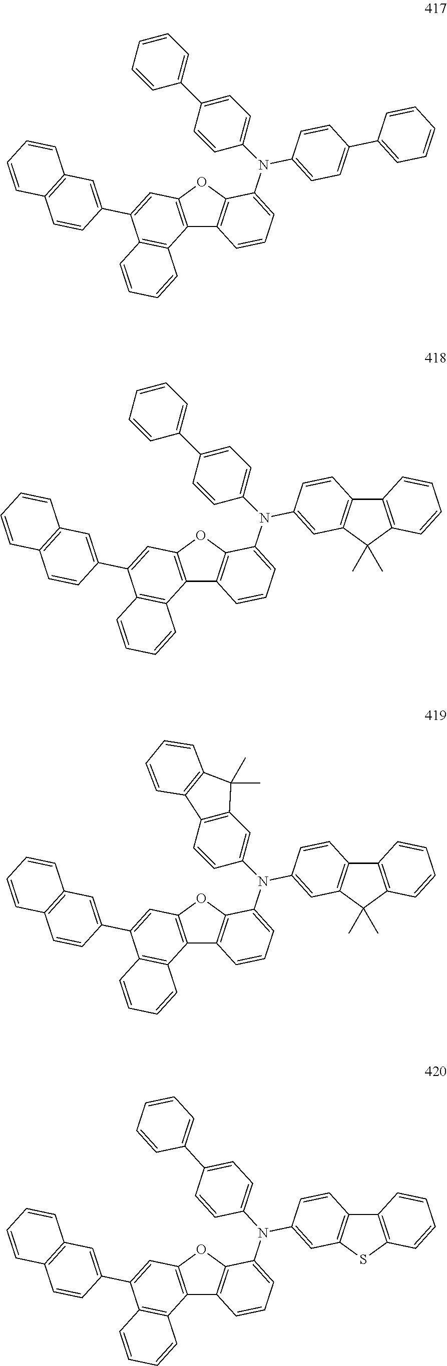 [SMILES: c1ccc(-c2ccc(N(c3ccc(-c4ccccc4)cc3)c3cccc4c3oc3cc(-c5ccc6ccccc6c5)c5ccccc5c34)cc2)cc1 ; CC1(C)c2ccccc2-c2ccc(N(c3ccc(-c4ccccc4)cc3)c3cccc4c3oc3cc(-c5ccc6ccccc6c5)c5ccccc5c34)cc21 ; CC1(C)c2ccccc2-c2ccc(N(c3ccc4c(c3)C(C)(C)c3ccccc3-4)c3cccc4c3oc3cc(-c5ccc6ccccc6c5)c5ccccc5c34)cc21 ; c1ccc(-c2ccc(N(c3ccc4c(c3)sc3ccccc34)c3cccc4c3oc3cc(-c5ccc6ccccc6c5)c5ccccc5c34)cc2)cc1]

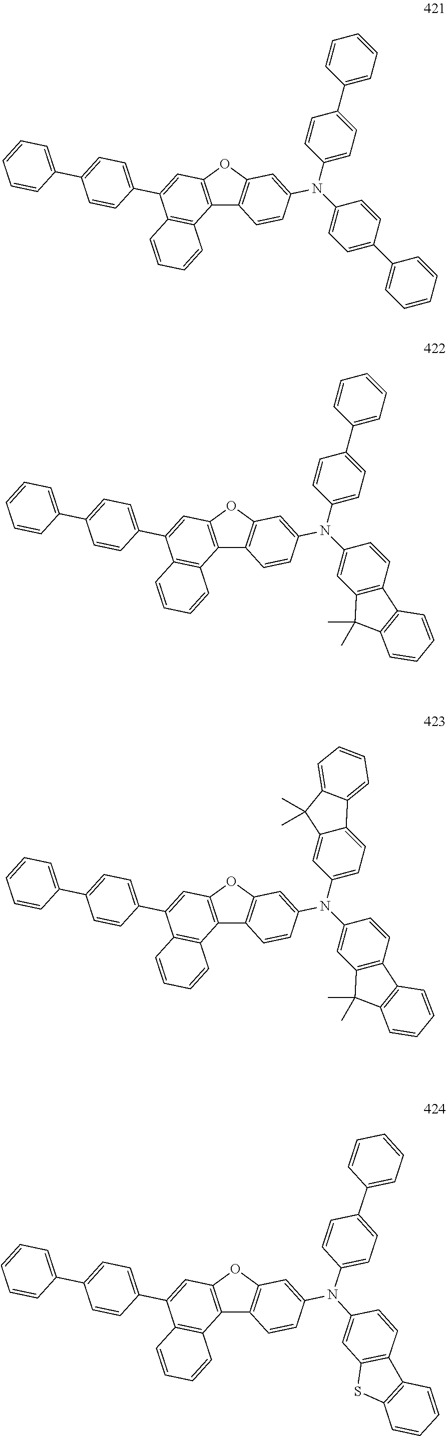 [SMILES: c1ccc(-c2ccc(-c3cc4oc5cc(N(c6ccc(-c7ccccc7)cc6)c6ccc(-c7ccccc7)cc6)ccc5c4c4ccccc34)cc2)cc1 ; CC1(C)c2ccccc2-c2ccc(N(c3ccc(-c4ccccc4)cc3)c3ccc4c(c3)oc3cc(-c5ccc(-c6ccccc6)cc5)c5ccccc5c34)cc21 ; CC1(C)c2ccccc2-c2ccc(N(c3ccc4c(c3)C(C)(C)c3ccccc3-4)c3ccc4c(c3)oc3cc(-c5ccc(-c6ccccc6)cc5)c5ccccc5c34)cc21 ; c1ccc(-c2ccc(-c3cc4oc5cc(N(c6ccc(-c7ccccc7)cc6)c6ccc7c(c6)sc6ccccc67)ccc5c4c4ccccc34)cc2)cc1]

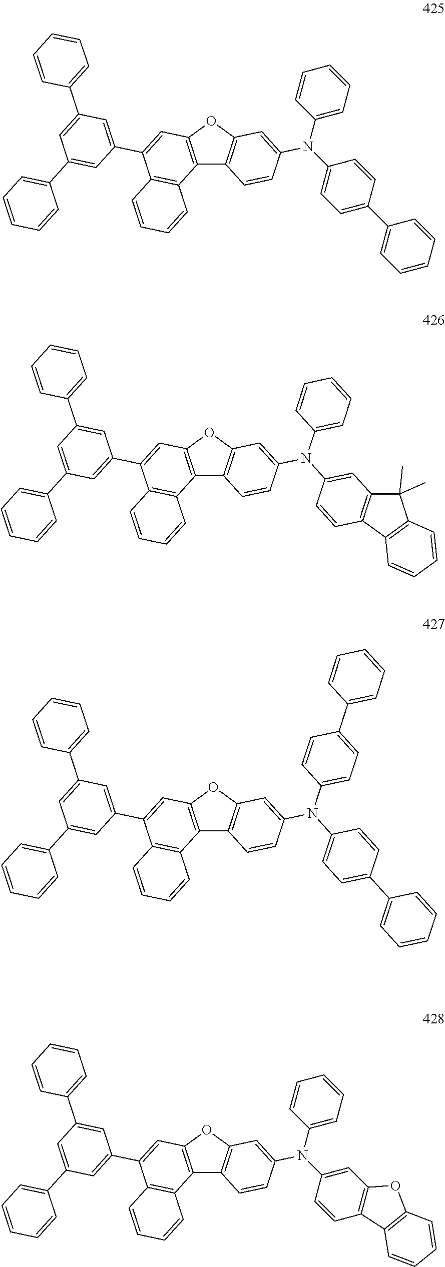 [SMILES: c1ccc(-c2ccc(N(c3ccccc3)c3ccc4c(c3)oc3cc(-c5cc(-c6ccccc6)cc(-c6ccccc6)c5)c5ccccc5c34)cc2)cc1 ; CC1(C)c2ccccc2-c2ccc(N(c3ccccc3)c3ccc4c(c3)oc3cc(-c5cc(-c6ccccc6)cc(-c6ccccc6)c5)c5ccccc5c34)cc21 ; c1ccc(-c2ccc(N(c3ccc(-c4ccccc4)cc3)c3ccc4c(c3)oc3cc(-c5cc(-c6ccccc6)cc(-c6ccccc6)c5)c5ccccc5c34)cc2)cc1 ; c1ccc(-c2cc(-c3ccccc3)cc(-c3cc4oc5cc(N(c6ccccc6)c6ccc7c(c6)oc6ccccc67)ccc5c4c4ccccc34)c2)cc1]

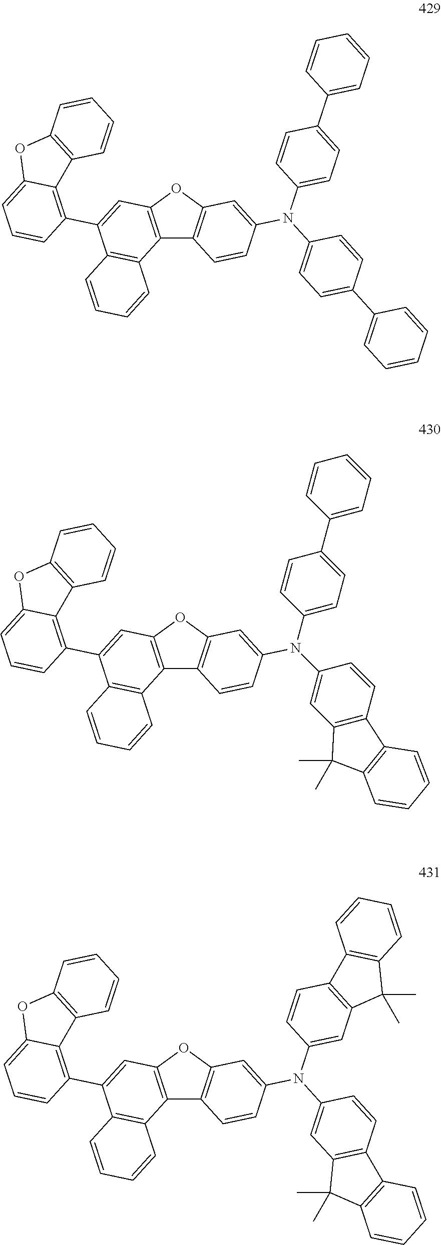 [SMILES: c1ccc(-c2ccc(N(c3ccc(-c4ccccc4)cc3)c3ccc4c(c3)oc3cc(-c5cccc6oc7ccccc7c56)c5ccccc5c34)cc2)cc1 ; CC1(C)c2ccccc2-c2ccc(N(c3ccc(-c4ccccc4)cc3)c3ccc4c(c3)oc3cc(-c5cccc6oc7ccccc7c56)c5ccccc5c34)cc21 ; CC1(C)c2ccccc2-c2ccc(N(c3ccc4c(c3)C(C)(C)c3ccccc3-4)c3ccc4c(c3)oc3cc(-c5cccc6oc7ccccc7c56)c5ccccc5c34)cc21]

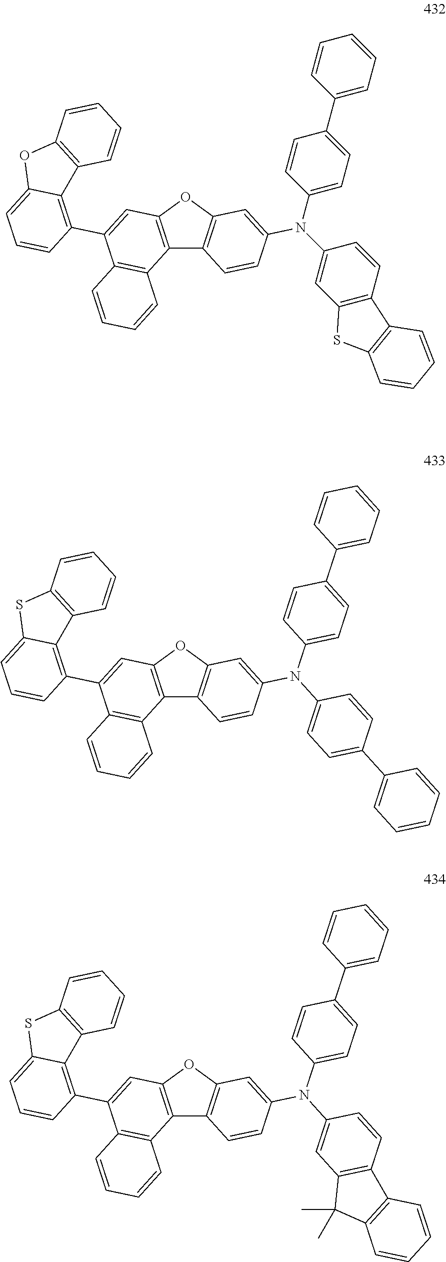 [SMILES: c1ccc(-c2ccc(N(c3ccc4c(c3)oc3cc(-c5cccc6oc7ccccc7c56)c5ccccc5c34)c3ccc4c(c3)sc3ccccc34)cc2)cc1 ; c1ccc(-c2ccc(N(c3ccc(-c4ccccc4)cc3)c3ccc4c(c3)oc3cc(-c5cccc6sc7ccccc7c56)c5ccccc5c34)cc2)cc1 ; CC1(C)c2ccccc2-c2ccc(N(c3ccc(-c4ccccc4)cc3)c3ccc4c(c3)oc3cc(-c5cccc6sc7ccccc7c56)c5ccccc5c34)cc21]

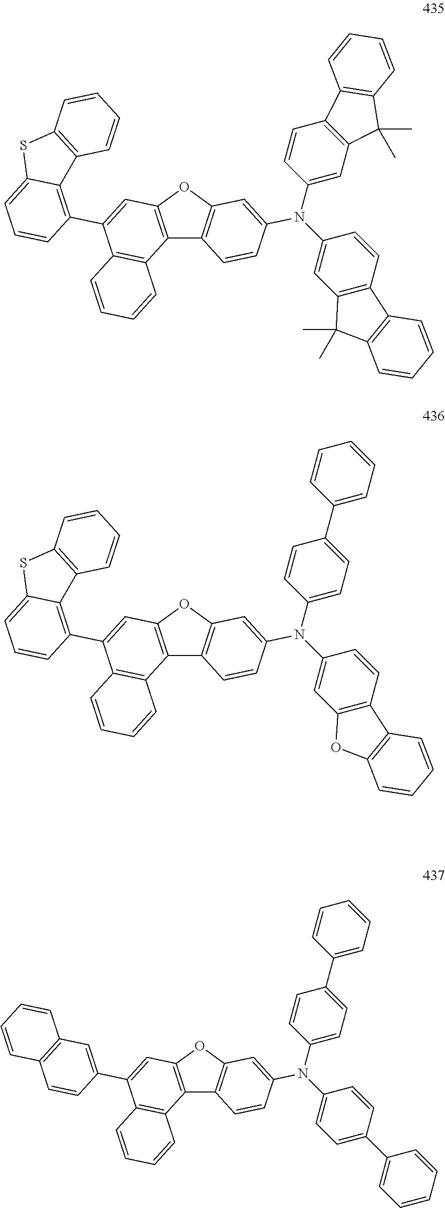 [SMILES: CC1(C)c2ccccc2-c2ccc(N(c3ccc4c(c3)C(C)(C)c3ccccc3-4)c3ccc4c(c3)oc3cc(-c5cccc6sc7ccccc7c56)c5ccccc5c34)cc21 ; c1ccc(-c2ccc(N(c3ccc4c(c3)oc3ccccc34)c3ccc4c(c3)oc3cc(-c5cccc6sc7ccccc7c56)c5ccccc5c34)cc2)cc1 ; c1ccc(-c2ccc(N(c3ccc(-c4ccccc4)cc3)c3ccc4c(c3)oc3cc(-c5ccc6ccccc6c5)c5ccccc5c34)cc2)cc1]

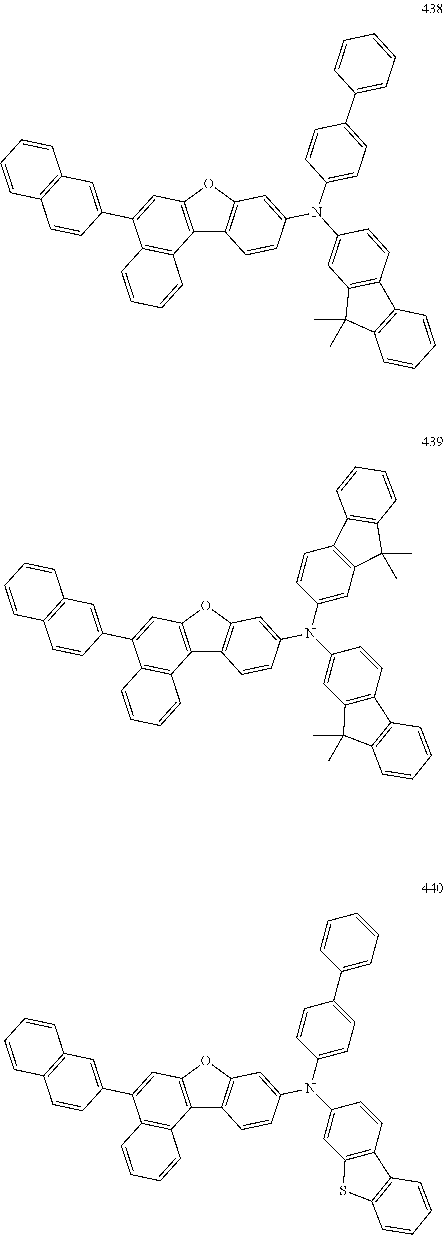 [SMILES: CC1(C)c2ccccc2-c2ccc(N(c3ccc(-c4ccccc4)cc3)c3ccc4c(c3)oc3cc(-c5ccc6ccccc6c5)c5ccccc5c34)cc21 ; CC1(C)c2ccccc2-c2ccc(N(c3ccc4c(c3)C(C)(C)c3ccccc3-4)c3ccc4c(c3)oc3cc(-c5ccc6ccccc6c5)c5ccccc5c34)cc21 ; c1ccc(-c2ccc(N(c3ccc4c(c3)oc3cc(-c5ccc6ccccc6c5)c5ccccc5c34)c3ccc4c(c3)sc3ccccc34)cc2)cc1]

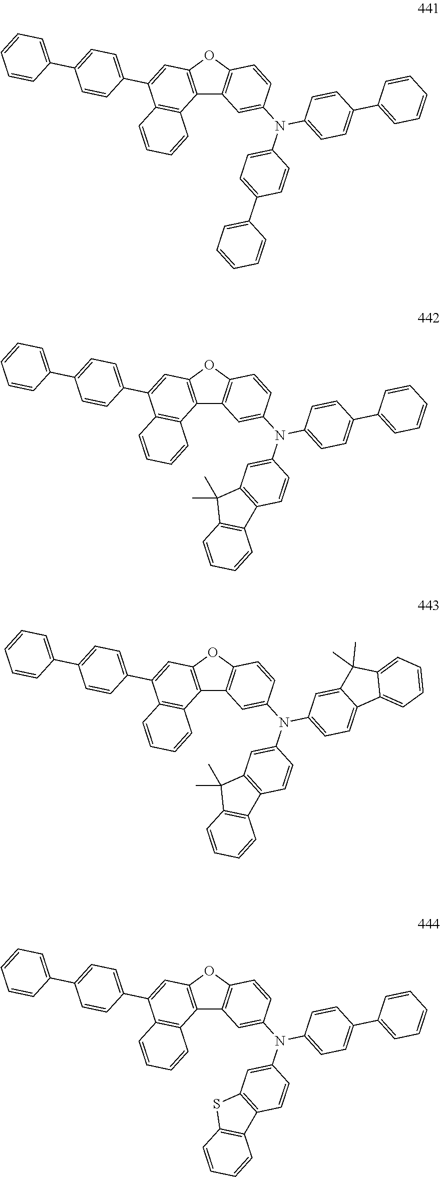 [SMILES: c1ccc(-c2ccc(-c3cc4oc5ccc(N(c6ccc(-c7ccccc7)cc6)c6ccc(-c7ccccc7)cc6)cc5c4c4ccccc34)cc2)cc1 ; CC1(C)c2ccccc2-c2ccc(N(c3ccc(-c4ccccc4)cc3)c3ccc4oc5cc(-c6ccc(-c7ccccc7)cc6)c6ccccc6c5c4c3)cc21 ; CC1(C)c2ccccc2-c2ccc(N(c3ccc4c(c3)C(C)(C)c3ccccc3-4)c3ccc4oc5cc(-c6ccc(-c7ccccc7)cc6)c6ccccc6c5c4c3)cc21 ; c1ccc(-c2ccc(-c3cc4oc5ccc(N(c6ccc(-c7ccccc7)cc6)c6ccc7c(c6)sc6ccccc67)cc5c4c4ccccc34)cc2)cc1]

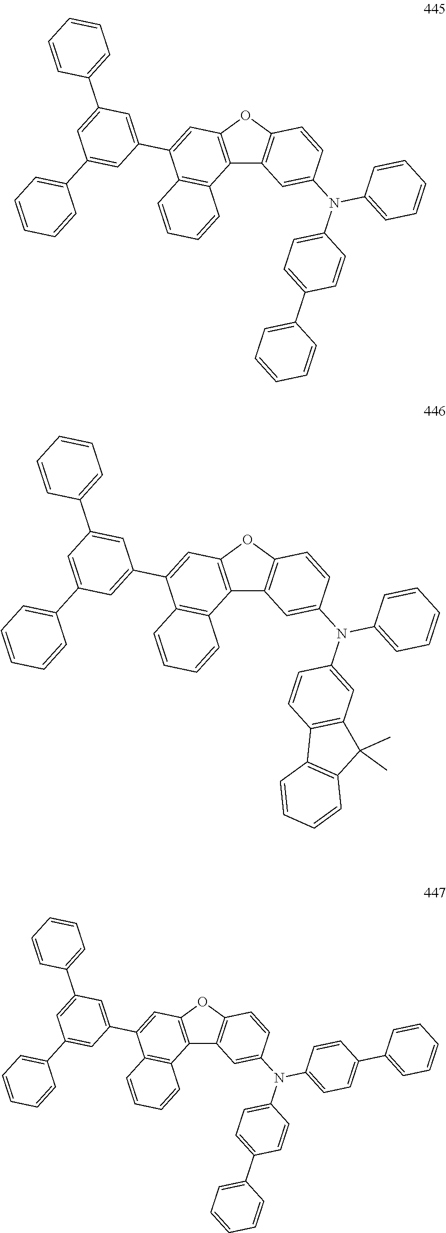 [SMILES: c1ccc(-c2ccc(N(c3ccccc3)c3ccc4oc5cc(-c6cc(-c7ccccc7)cc(-c7ccccc7)c6)c6ccccc6c5c4c3)cc2)cc1 ; CC1(C)c2ccccc2-c2ccc(N(c3ccccc3)c3ccc4oc5cc(-c6cc(-c7ccccc7)cc(-c7ccccc7)c6)c6ccccc6c5c4c3)cc21 ; c1ccc(-c2ccc(N(c3ccc(-c4ccccc4)cc3)c3ccc4oc5cc(-c6cc(-c7ccccc7)cc(-c7ccccc7)c6)c6ccccc6c5c4c3)cc2)cc1]

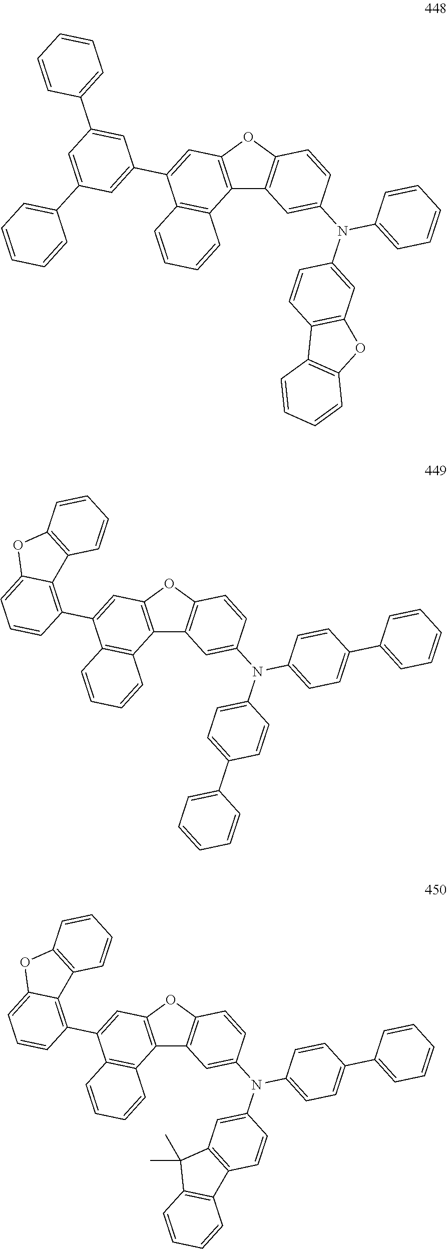 [SMILES: c1ccc(-c2cc(-c3ccccc3)cc(-c3cc4oc5ccc(N(c6ccccc6)c6ccc7c(c6)oc6ccccc67)cc5c4c4ccccc34)c2)cc1 ; c1ccc(-c2ccc(N(c3ccc(-c4ccccc4)cc3)c3ccc4oc5cc(-c6cccc7oc8ccccc8c67)c6ccccc6c5c4c3)cc2)cc1 ; CC1(C)c2ccccc2-c2ccc(N(c3ccc(-c4ccccc4)cc3)c3ccc4oc5cc(-c6cccc7oc8ccccc8c67)c6ccccc6c5c4c3)cc21]

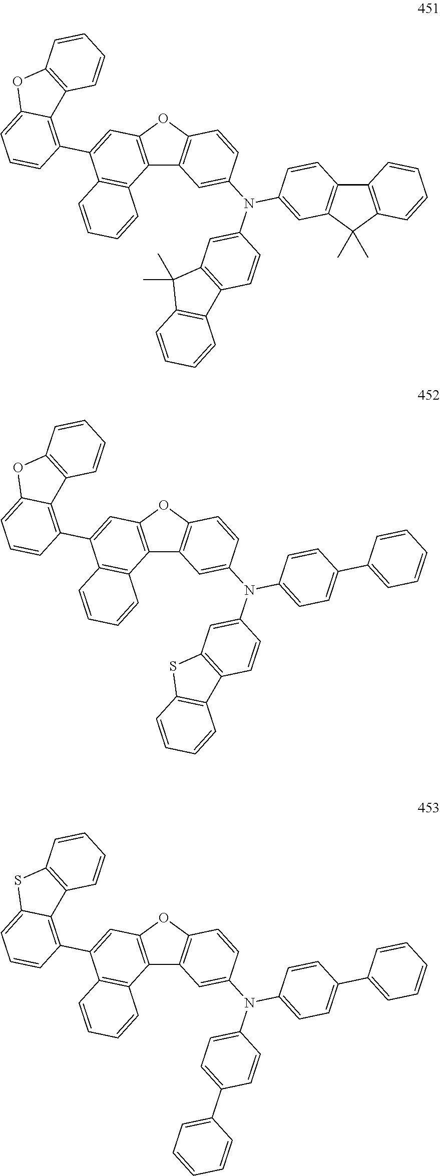 [SMILES: CC1(C)c2ccccc2-c2ccc(N(c3ccc4c(c3)C(C)(C)c3ccccc3-4)c3ccc4oc5cc(-c6cccc7oc8ccccc8c67)c6ccccc6c5c4c3)cc21 ; c1ccc(-c2ccc(N(c3ccc4c(c3)sc3ccccc34)c3ccc4oc5cc(-c6cccc7oc8ccccc8c67)c6ccccc6c5c4c3)cc2)cc1 ; c1ccc(-c2ccc(N(c3ccc(-c4ccccc4)cc3)c3ccc4oc5cc(-c6cccc7sc8ccccc8c67)c6ccccc6c5c4c3)cc2)cc1]

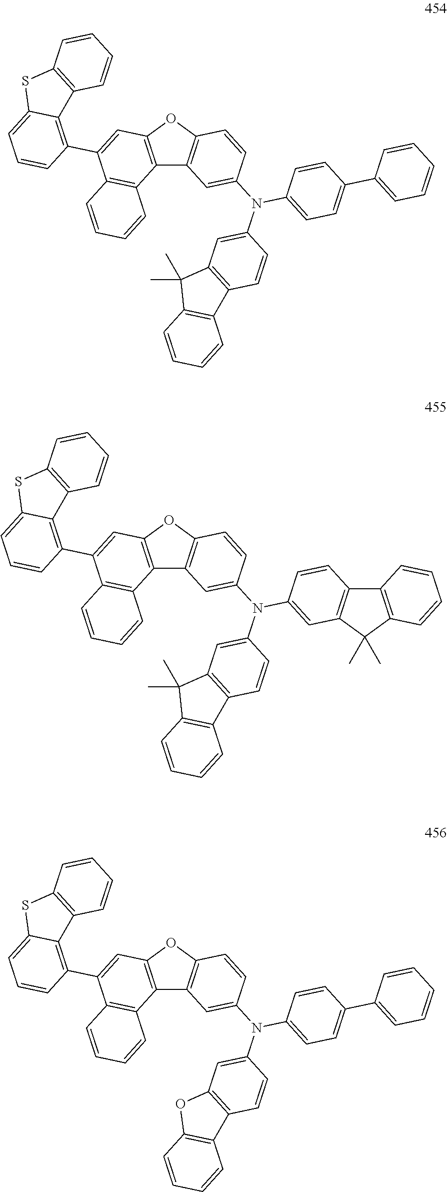 [SMILES: CC1(C)c2ccccc2-c2ccc(N(c3ccc(-c4ccccc4)cc3)c3ccc4oc5cc(-c6cccc7sc8ccccc8c67)c6ccccc6c5c4c3)cc21 ; CC1(C)c2ccccc2-c2ccc(N(c3ccc4c(c3)C(C)(C)c3ccccc3-4)c3ccc4oc5cc(-c6cccc7sc8ccccc8c67)c6ccccc6c5c4c3)cc21 ; c1ccc(-c2ccc(N(c3ccc4c(c3)oc3ccccc34)c3ccc4oc5cc(-c6cccc7sc8ccccc8c67)c6ccccc6c5c4c3)cc2)cc1]

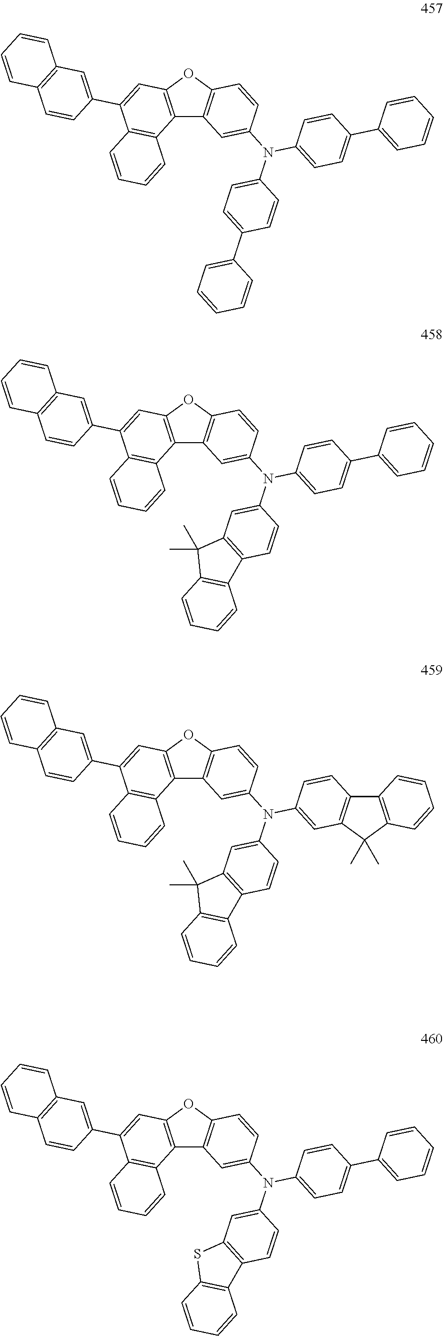 [SMILES: c1ccc(-c2ccc(N(c3ccc(-c4ccccc4)cc3)c3ccc4oc5cc(-c6ccc7ccccc7c6)c6ccccc6c5c4c3)cc2)cc1 ; CC1(C)c2ccccc2-c2ccc(N(c3ccc(-c4ccccc4)cc3)c3ccc4oc5cc(-c6ccc7ccccc7c6)c6ccccc6c5c4c3)cc21 ; CC1(C)c2ccccc2-c2ccc(N(c3ccc4c(c3)C(C)(C)c3ccccc3-4)c3ccc4oc5cc(-c6ccc7ccccc7c6)c6ccccc6c5c4c3)cc21 ; c1ccc(-c2ccc(N(c3ccc4c(c3)sc3ccccc34)c3ccc4oc5cc(-c6ccc7ccccc7c6)c6ccccc6c5c4c3)cc2)cc1]

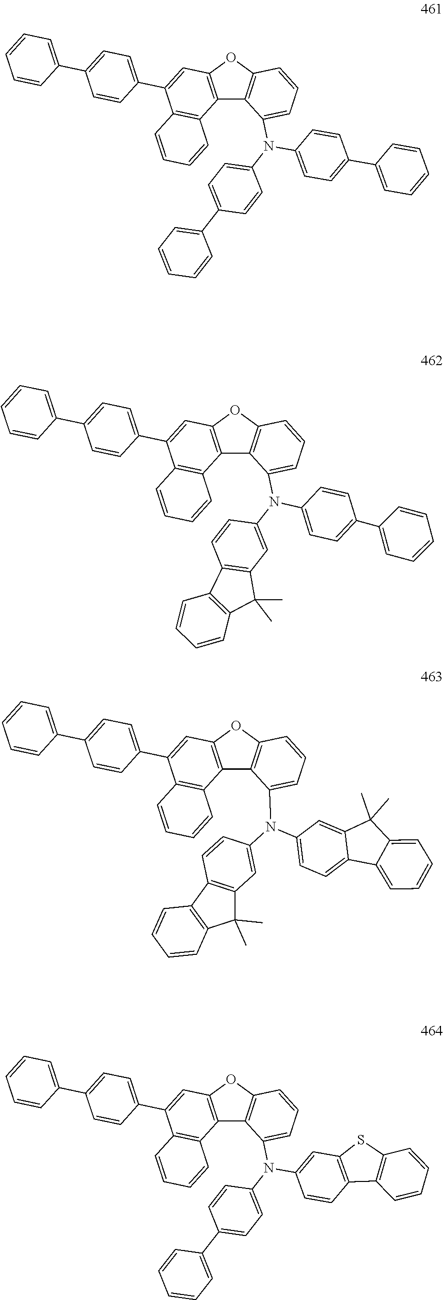 [SMILES: c1ccc(-c2ccc(-c3cc4oc5cccc(N(c6ccc(-c7ccccc7)cc6)c6ccc(-c7ccccc7)cc6)c5c4c4ccccc34)cc2)cc1 ; CC1(C)c2ccccc2-c2ccc(N(c3ccc(-c4ccccc4)cc3)c3cccc4oc5cc(-c6ccc(-c7ccccc7)cc6)c6ccccc6c5c34)cc21 ; CC1(C)c2ccccc2-c2ccc(N(c3ccc4c(c3)C(C)(C)c3ccccc3-4)c3cccc4oc5cc(-c6ccc(-c7ccccc7)cc6)c6ccccc6c5c34)cc21 ; c1ccc(-c2ccc(-c3cc4oc5cccc(N(c6ccc(-c7ccccc7)cc6)c6ccc7c(c6)sc6ccccc67)c5c4c4ccccc34)cc2)cc1]

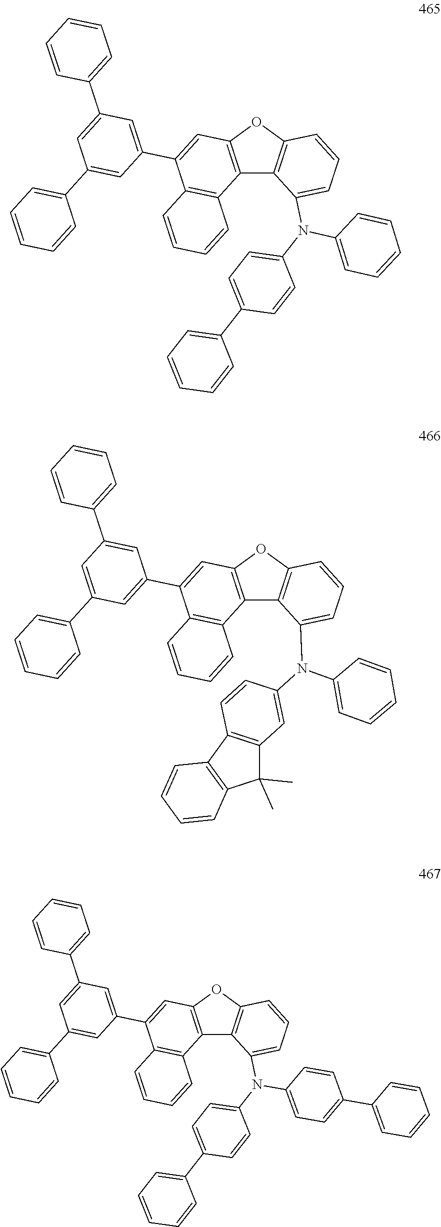 [SMILES: c1ccc(-c2ccc(N(c3ccccc3)c3cccc4oc5cc(-c6cc(-c7ccccc7)cc(-c7ccccc7)c6)c6ccccc6c5c34)cc2)cc1 ; CC1(C)c2ccccc2-c2ccc(N(c3ccccc3)c3cccc4oc5cc(-c6cc(-c7ccccc7)cc(-c7ccccc7)c6)c6ccccc6c5c34)cc21 ; c1ccc(-c2ccc(N(c3ccc(-c4ccccc4)cc3)c3cccc4oc5cc(-c6cc(-c7ccccc7)cc(-c7ccccc7)c6)c6ccccc6c5c34)cc2)cc1]

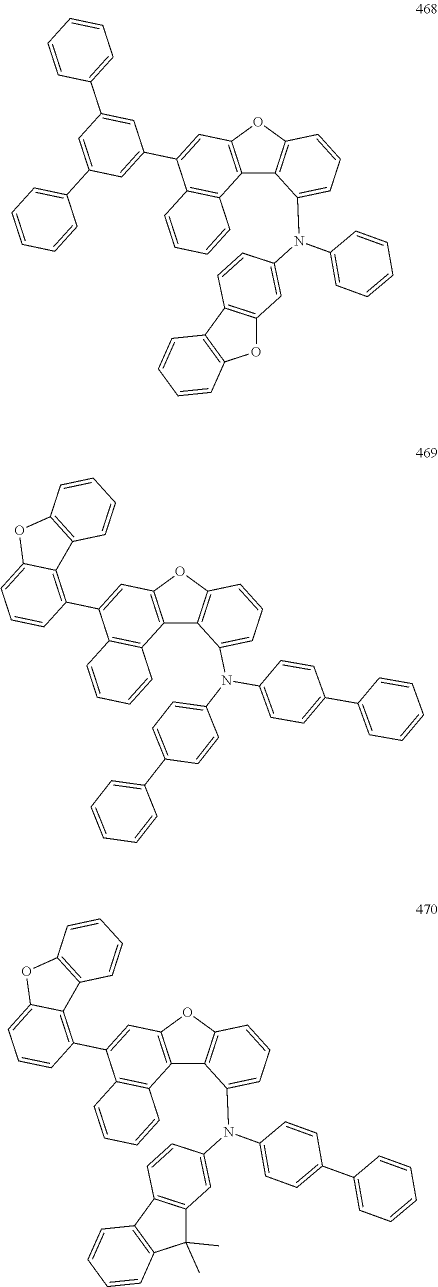 [SMILES: c1ccc(-c2cc(-c3ccccc3)cc(-c3cc4oc5cccc(N(c6ccccc6)c6ccc7c(c6)oc6ccccc67)c5c4c4ccccc34)c2)cc1 ; c1ccc(-c2ccc(N(c3ccc(-c4ccccc4)cc3)c3cccc4oc5cc(-c6cccc7oc8ccccc8c67)c6ccccc6c5c34)cc2)cc1 ; CC1(C)c2ccccc2-c2ccc(N(c3ccc(-c4ccccc4)cc3)c3cccc4oc5cc(-c6cccc7oc8ccccc8c67)c6ccccc6c5c34)cc21]

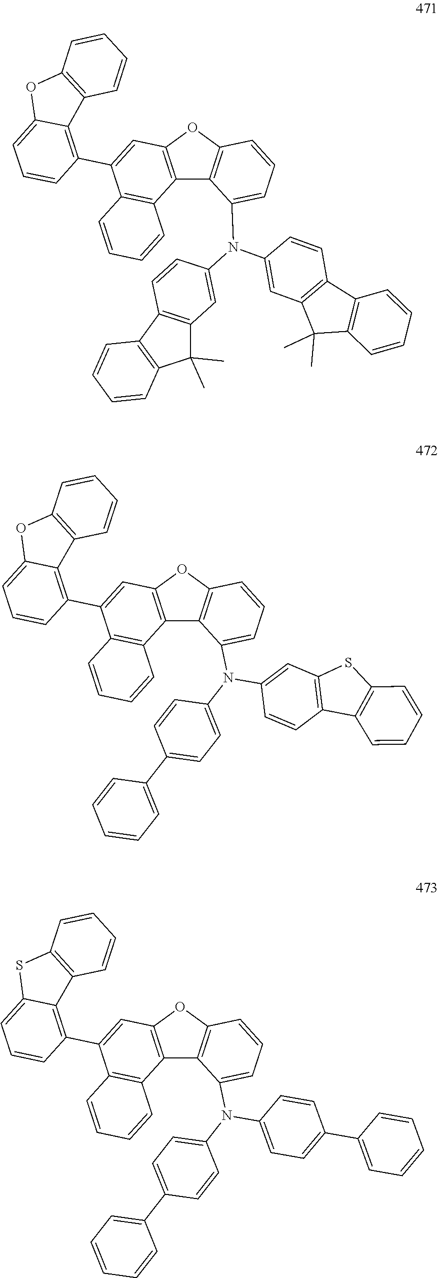 [SMILES: CC1(C)c2ccccc2-c2ccc(N(c3ccc4c(c3)C(C)(C)c3ccccc3-4)c3cccc4oc5cc(-c6cccc7oc8ccccc8c67)c6ccccc6c5c34)cc21 ; c1ccc(-c2ccc(N(c3ccc4c(c3)sc3ccccc34)c3cccc4oc5cc(-c6cccc7oc8ccccc8c67)c6ccccc6c5c34)cc2)cc1 ; c1ccc(-c2ccc(N(c3ccc(-c4ccccc4)cc3)c3cccc4oc5cc(-c6cccc7sc8ccccc8c67)c6ccccc6c5c34)cc2)cc1]

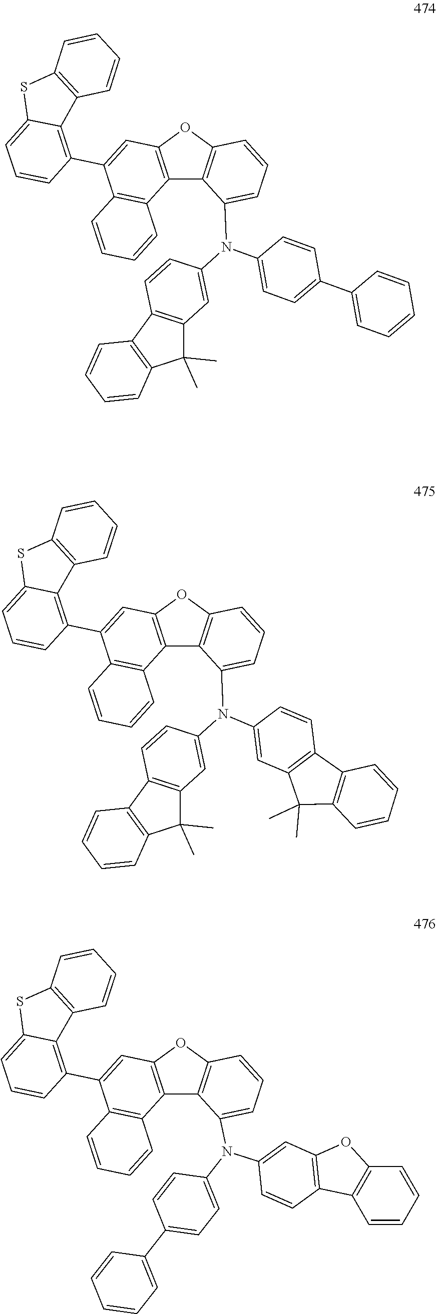 [SMILES: CC1(C)c2ccccc2-c2ccc(N(c3ccc(-c4ccccc4)cc3)c3cccc4oc5cc(-c6cccc7sc8ccccc8c67)c6ccccc6c5c34)cc21 ; CC1(C)c2ccccc2-c2ccc(N(c3ccc4c(c3)C(C)(C)c3ccccc3-4)c3cccc4oc5cc(-c6cccc7sc8ccccc8c67)c6ccccc6c5c34)cc21 ; c1ccc(-c2ccc(N(c3ccc4c(c3)oc3ccccc34)c3cccc4oc5cc(-c6cccc7sc8ccccc8c67)c6ccccc6c5c34)cc2)cc1]

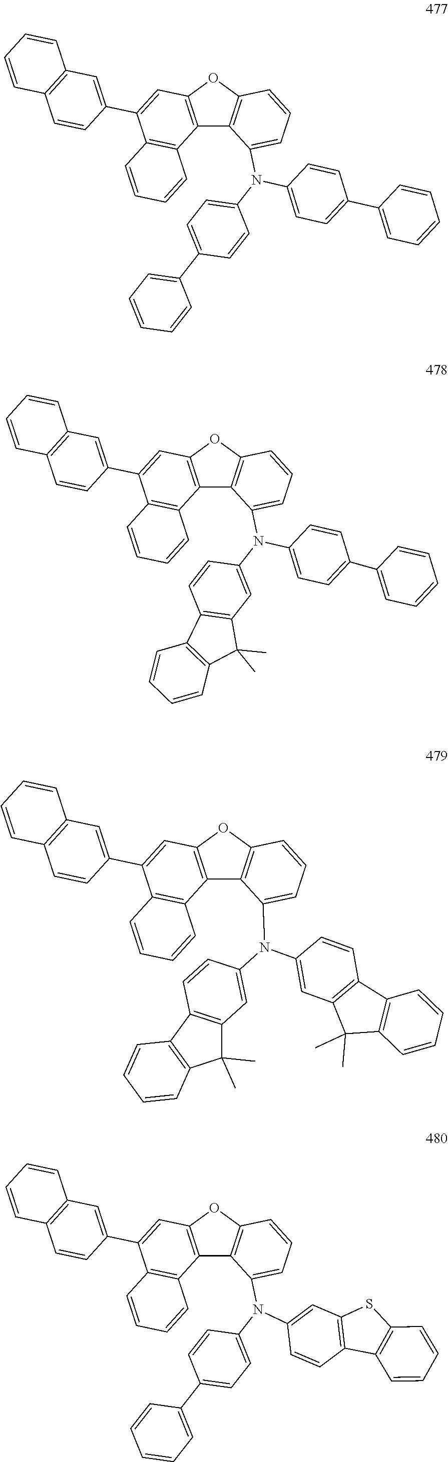 [SMILES: c1ccc(-c2ccc(N(c3ccc(-c4ccccc4)cc3)c3cccc4oc5cc(-c6ccc7ccccc7c6)c6ccccc6c5c34)cc2)cc1 ; CC1(C)c2ccccc2-c2ccc(N(c3ccc(-c4ccccc4)cc3)c3cccc4oc5cc(-c6ccc7ccccc7c6)c6ccccc6c5c34)cc21 ; CC1(C)c2ccccc2-c2ccc(N(c3ccc4c(c3)C(C)(C)c3ccccc3-4)c3cccc4oc5cc(-c6ccc7ccccc7c6)c6ccccc6c5c34)cc21 ; c1ccc(-c2ccc(N(c3ccc4c(c3)sc3ccccc34)c3cccc4oc5cc(-c6ccc7ccccc7c6)c6ccccc6c5c34)cc2)cc1]

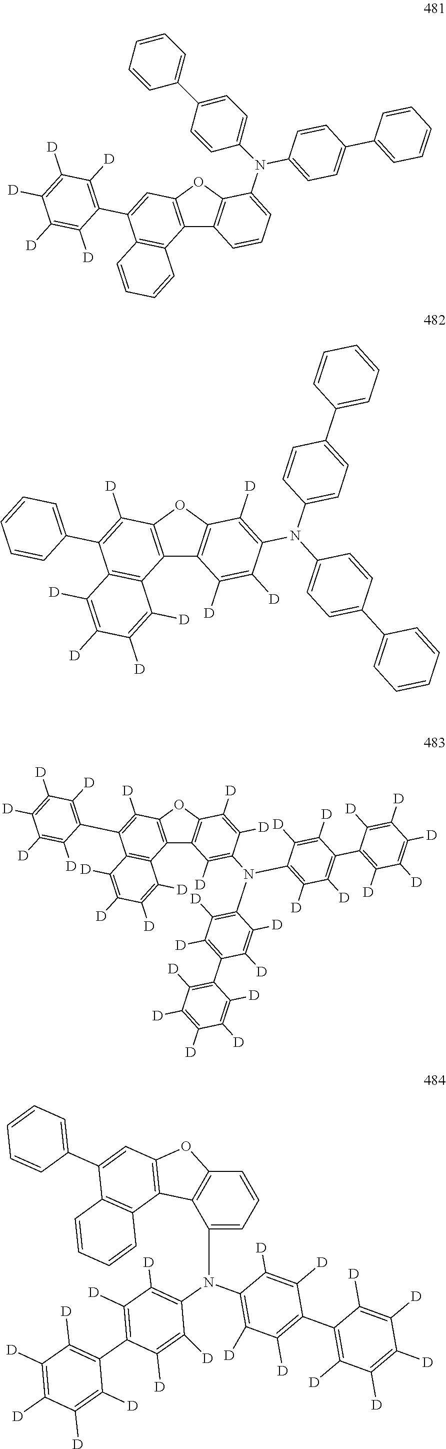

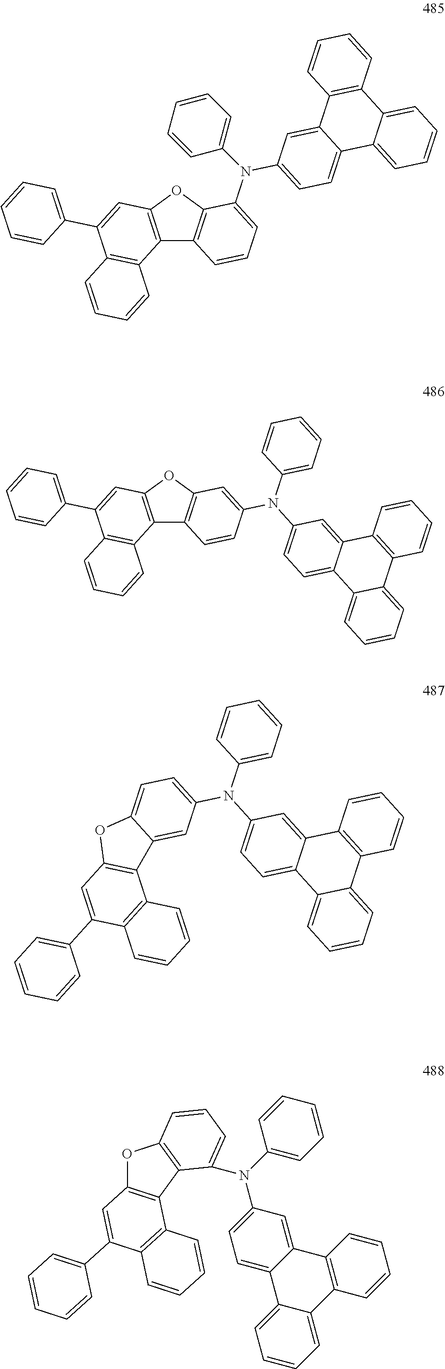 [SMILES: c1ccc(-c2cc3oc4c(N(c5ccccc5)c5ccc6c7ccccc7c7ccccc7c6c5)cccc4c3c3ccccc23)cc1 ; c1ccc(-c2cc3oc4cc(N(c5ccccc5)c5ccc6c7ccccc7c7ccccc7c6c5)ccc4c3c3ccccc23)cc1 ; c1ccc(-c2cc3oc4ccc(N(c5ccccc5)c5ccc6c7ccccc7c7ccccc7c6c5)cc4c3c3ccccc23)cc1 ; c1ccc(-c2cc3oc4cccc(N(c5ccccc5)c5ccc6c7ccccc7c7ccccc7c6c5)c4c3c3ccccc23)cc1]

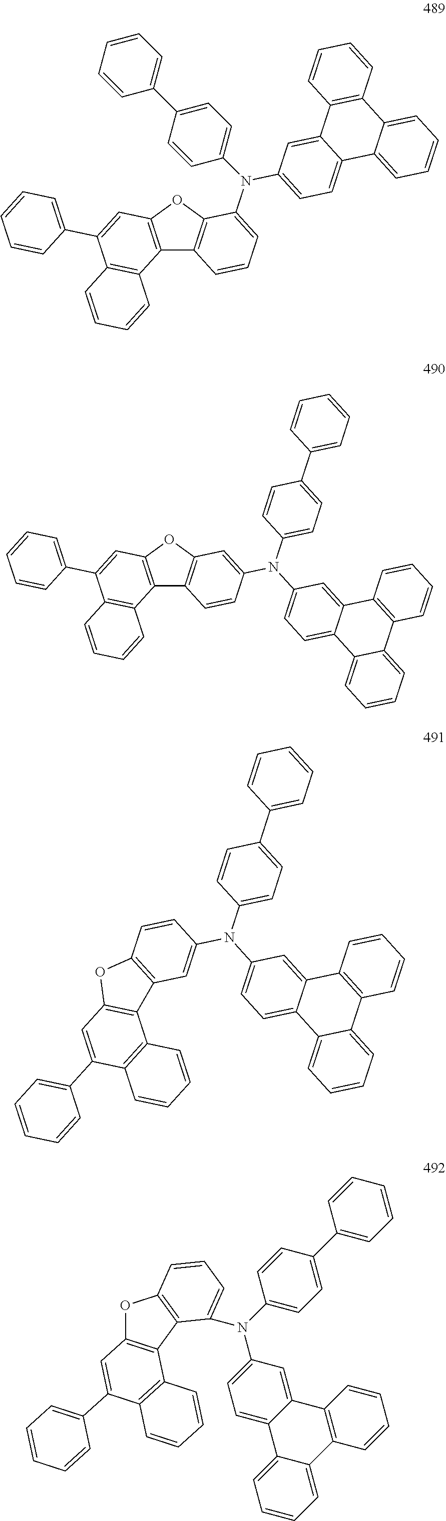 [SMILES: c1ccc(-c2ccc(N(c3ccc4c5ccccc5c5ccccc5c4c3)c3cccc4c3oc3cc(-c5ccccc5)c5ccccc5c34)cc2)cc1 ; c1ccc(-c2ccc(N(c3ccc4c(c3)oc3cc(-c5ccccc5)c5ccccc5c34)c3ccc4c5ccccc5c5ccccc5c4c3)cc2)cc1 ; c1ccc(-c2ccc(N(c3ccc4c5ccccc5c5ccccc5c4c3)c3ccc4oc5cc(-c6ccccc6)c6ccccc6c5c4c3)cc2)cc1 ; c1ccc(-c2ccc(N(c3ccc4c5ccccc5c5ccccc5c4c3)c3cccc4oc5cc(-c6ccccc6)c6ccccc6c5c34)cc2)cc1]

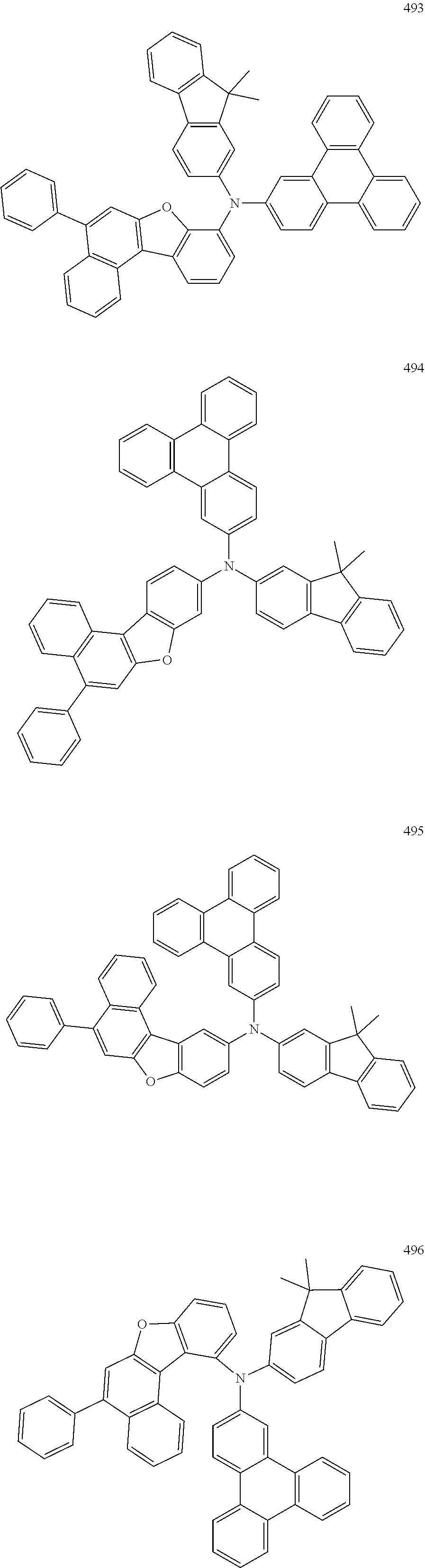 [SMILES: CC1(C)c2ccccc2-c2ccc(N(c3ccc4c5ccccc5c5ccccc5c4c3)c3cccc4c3oc3cc(-c5ccccc5)c5ccccc5c34)cc21 ; CC1(C)c2ccccc2-c2ccc(N(c3ccc4c(c3)oc3cc(-c5ccccc5)c5ccccc5c34)c3ccc4c5ccccc5c5ccccc5c4c3)cc21 ; CC1(C)c2ccccc2-c2ccc(N(c3ccc4c5ccccc5c5ccccc5c4c3)c3ccc4oc5cc(-c6ccccc6)c6ccccc6c5c4c3)cc21 ; CC1(C)c2ccccc2-c2ccc(N(c3ccc4c5ccccc5c5ccccc5c4c3)c3cccc4oc5cc(-c6ccccc6)c6ccccc6c5c34)cc21]

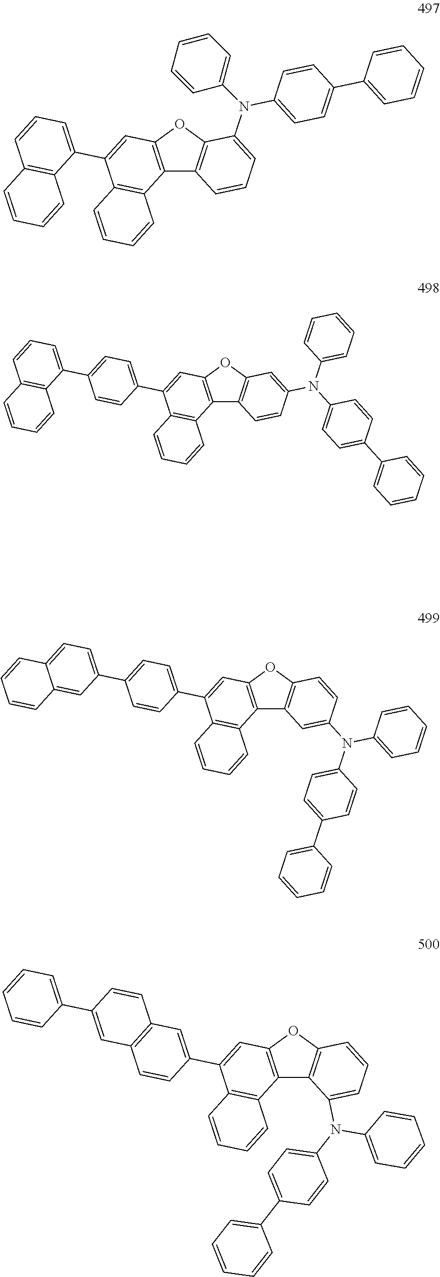 [SMILES: c1ccc(-c2ccc(N(c3ccccc3)c3cccc4c3oc3cc(-c5cccc6ccccc56)c5ccccc5c34)cc2)cc1 ; c1ccc(-c2ccc(N(c3ccccc3)c3ccc4c(c3)oc3cc(-c5ccc(-c6cccc7ccccc67)cc5)c5ccccc5c34)cc2)cc1 ; c1ccc(-c2ccc(N(c3ccccc3)c3ccc4oc5cc(-c6ccc(-c7ccc8ccccc8c7)cc6)c6ccccc6c5c4c3)cc2)cc1 ; c1ccc(-c2ccc(N(c3ccccc3)c3cccc4oc5cc(-c6ccc7cc(-c8ccccc8)ccc7c6)c6ccccc6c5c34)cc2)cc1]

Further, various substituents may be introduced into the structure of Chemical Formula 1 to synthesize a compound having inherent characteristics of a substituent introduced. For example, it is possible to synthesize a material which satisfies conditions required for each organic material layer by introducing a substituent usually used for a hole injection layer material, a hole transport layer material, a light emitting layer material, an electron transport layer material, and a charge generation layer material, which are used for preparing an organic light emitting device, into the core structure.

In addition, it is possible to finely adjust an energy band-gap by introducing various substituents into the structure of Chemical Formula 1, and meanwhile, it is possible to improve characteristics at the interface between organic materials and diversify the use of the material.

In another exemplary embodiment of the present specification, provided is an organic light emitting device including: a first electrode; a second electrode; and an organic material layer having one or more layers provided between the first electrode and the second electrode, in which one or more layers of the organic material layer include the one or more heterocyclic compounds of Chemical Formula 1.

In an exemplary embodiment of the present specification, the organic material layer includes a hole transport layer, and the hole transport layer may include one or more of the heterocyclic compounds.

In an exemplary embodiment of the present specification, the organic material layer includes an electron blocking layer, and the electron blocking layer may include one or more of the heterocyclic compounds.

In an exemplary embodiment of the present specification, the organic material layer includes a light emitting layer, and the light emitting layer may include one or more of the heterocyclic compounds.

In an exemplary embodiment of the present specification, the organic material layer includes a light emitting layer, and the light emitting layer may include one of the heterocyclic compounds.

In an exemplary embodiment of the present specification, the organic material layer includes a light emitting layer, the light emitting layer includes a host, and the host may include one or more of the heterocyclic compounds.

In an exemplary embodiment of the present specification, the organic material layer includes a light emitting layer, the light emitting layer includes a host, and the host may include one of the heterocyclic compounds.

In an exemplary embodiment of the present specification, the organic material layer includes a light emitting layer, the light emitting layer includes a host, the host includes a green host, and the green host may include one or more of the heterocyclic compounds.

In an exemplary embodiment of the present specification, the organic material layer includes a light emitting layer, the light emitting layer includes a host, the host includes a red host, and the red host may include one or more of the heterocyclic compounds.

In an exemplary embodiment of the present specification, the organic material layer includes a light emitting layer, the light emitting layer includes a host, the host includes a blue host, and the blue host may include one or more of the heterocyclic compounds.

In an exemplary embodiment of the present specification, the organic material layer includes a light emitting layer, and the light emitting layer may include the heterocyclic compound as a P-type host.

In an exemplary embodiment of the present specification, the organic material layer includes a light emitting layer, and the light emitting layer may further include a compound of the following Chemical Formula 2 in addition to the heterocyclic compound.

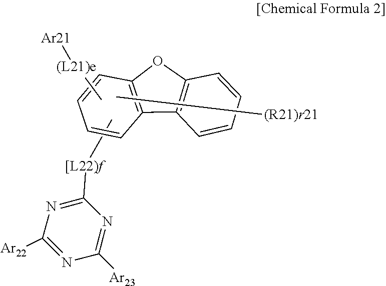

In Chemical Formula 2,
L21 and L22 are each independently a direct bond; or a substituted or unsubstituted C6 to C60 arylene group,
e and f are an integer from 0 to 4,
when e and f are 2 or higher, substituents in the parenthesis are the same as or different from each other,
Ar21 is a substituted or unsubstituted C6 to C60 aryl group,
Ar22 and Ar23 are each independently a substituted or unsubstituted C6 to C60 aryl group; or a substituted or unsubstituted C2 to C60 heteroaryl group,
R21 is hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, and
r21 is an integer from 0 to 6, and when r21 is 2 or higher, R21's are the same as or different from each other.

In an exemplary embodiment of the present specification, the light emitting layer may further include the compound of Chemical Formula 2 as an N-type host in addition to the heterocyclic compound.

In an exemplary embodiment of the present specification, L21 and L22 are each independently a direct bond; or a substituted or unsubstituted C6 to C60 arylene group.

In an exemplary embodiment of the present specification, L21 and L22 may be each independently a direct bond; or a substituted or unsubstituted C6 to C30 arylene group.

In an exemplary embodiment of the present specification, L21 and L22 may be each independently a direct bond; a substituted or unsubstituted phenylene group; or a substituted or unsubstituted biphenylene group.

In an exemplary embodiment of the present specification, L21 and L22 may be a direct bond.

In an exemplary embodiment of the present specification, Ar21 is a substituted or unsubstituted C6 to C60 aryl group.

In an exemplary embodiment of the present specification, Ar21 may be a substituted or unsubstituted C6 to C30 aryl group.

In an exemplary embodiment of the present specification, Ar21 may be a substituted or unsubstituted C6 to C20 aryl group.

In an exemplary embodiment of the present specification, Ar21 may be a substituted or unsubstituted phenyl group; a substituted or unsubstituted naphthyl group; or a substituted or unsubstituted terphenyl group.

In an exemplary embodiment of the present specification, Ar22 and Ar23 are each independently a substituted or unsubstituted C6 to C60 aryl group; or a substituted or unsubstituted C2 to C60 heteroaryl group.

In an exemplary embodiment of the present specification, Ar22 and Ar23 may be each independently a substituted or unsubstituted C6 to C30 aryl group; or a substituted or unsubstituted C2 to C30 heteroaryl group.

In an exemplary embodiment of the present specification, Ar22 and Ar23 may be each independently a substituted or unsubstituted phenyl group; a substituted or unsubstituted biphenyl group; a substituted or unsubstituted terphenyl group; a substituted or unsubstituted naphthyl group; a substituted or unsubstituted fluorenyl group; a substituted or unsubstituted phenanthrene group; a substituted or unsubstituted triphenylene group; a substituted or unsubstituted chrysene group; a substituted or unsubstituted dibenzofuran group; or a substituted or unsubstituted dibenzothiophene group.

In an exemplary embodiment of the present specification, Ar22 and Ar23 may be each independently a phenyl group unsubstituted or substituted with an aryl group; a biphenyl group; a terphenyl group; a naphthyl group unsubstituted or substituted with an aryl group; a fluorenyl group unsubstituted or substituted with an alkyl group or an aryl group; a phenanthrene group; a triphenylene group; a chrysene group; a dibenzofuran group; or a dibenzothiophene group.

In an exemplary embodiment of the present specification, Ar22 is a substituted or unsubstituted C6 to C30 aryl group, and Ar23 may be a substituted or unsubstituted C6 to C30 aryl group; or a substituted or unsubstituted C2 to C30 heteroaryl group.

In an exemplary embodiment of the present specification, Chemical Formula 2 may be represented by any one of the following compounds.

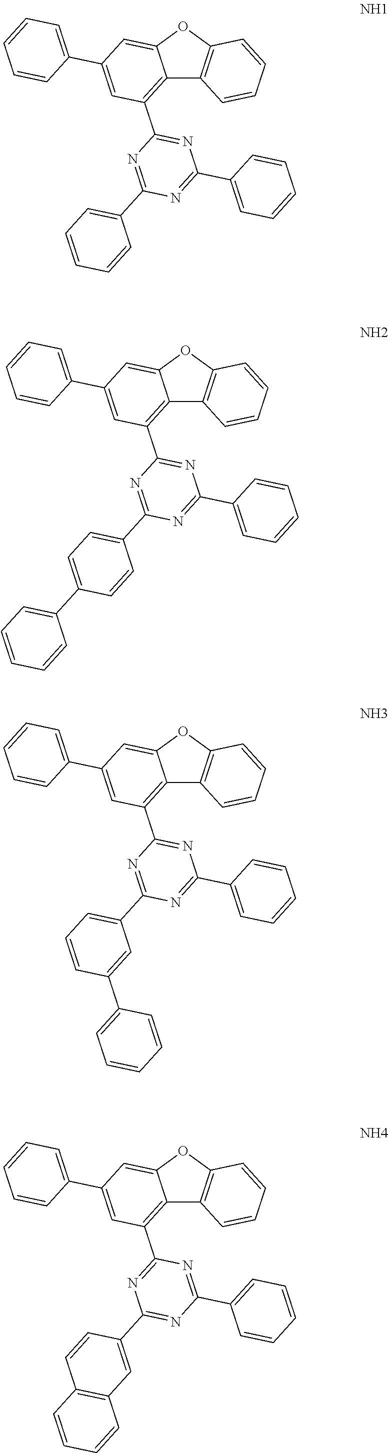 [SMILES: c1ccc(-c2cc(-c3nc(-c4ccccc4)nc(-c4ccccc4)n3)c3c(c2)oc2ccccc23)cc1 ; c1ccc(-c2ccc(-c3nc(-c4ccccc4)nc(-c4cc(-c5ccccc5)cc5oc6ccccc6c45)n3)cc2)cc1 ; c1ccc(-c2cccc(-c3nc(-c4ccccc4)nc(-c4cc(-c5ccccc5)cc5oc6ccccc6c45)n3)c2)cc1 ; c1ccc(-c2cc(-c3nc(-c4ccccc4)nc(-c4ccc5ccccc5c4)n3)c3c(c2)oc2ccccc23)cc1]

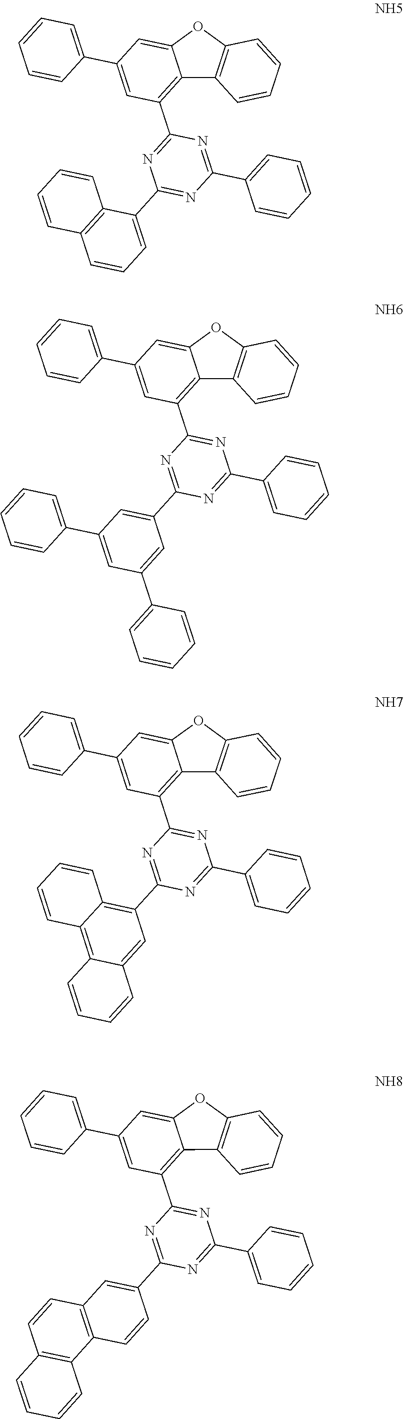 [SMILES: c1ccc(-c2cc(-c3nc(-c4ccccc4)nc(-c4cccc5ccccc45)n3)c3c(c2)oc2ccccc23)cc1 ; c1ccc(-c2cc(-c3ccccc3)cc(-c3nc(-c4ccccc4)nc(-c4cc(-c5ccccc5)cc5oc6ccccc6c45)n3)c2)cc1 ; c1ccc(-c2cc(-c3nc(-c4ccccc4)nc(-c4cc5ccccc5c5ccccc45)n3)c3c(c2)oc2ccccc23)cc1 ; c1ccc(-c2cc(-c3nc(-c4ccccc4)nc(-c4ccc5c(ccc6ccccc65)c4)n3)c3c(c2)oc2ccccc23)cc1]

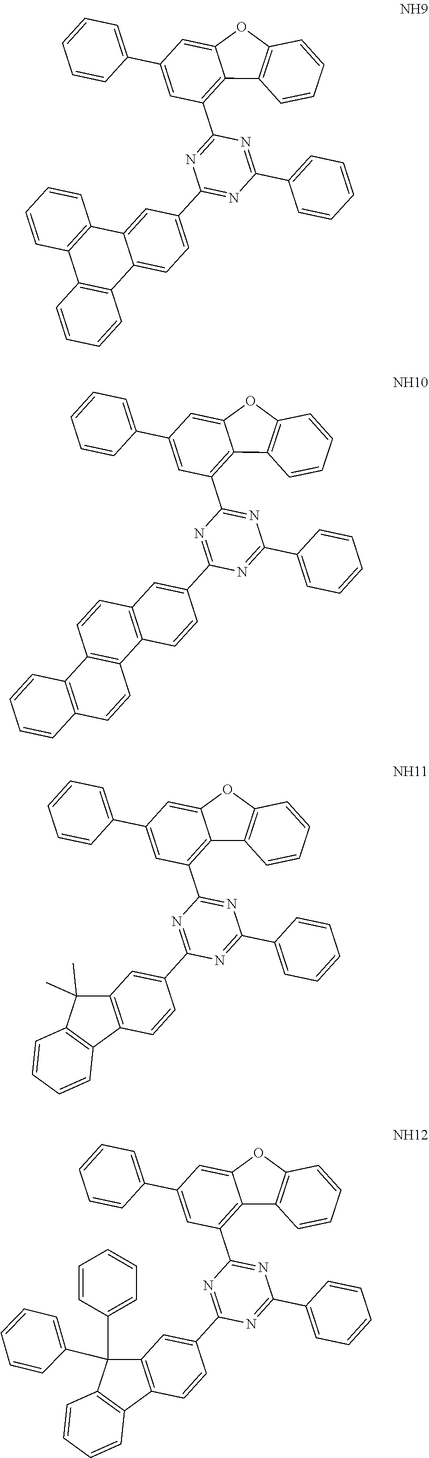

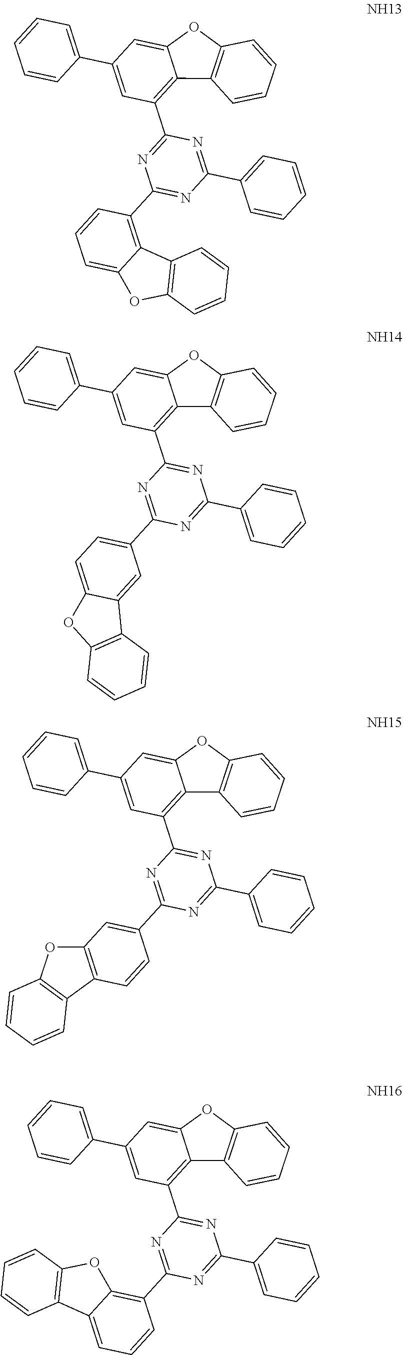

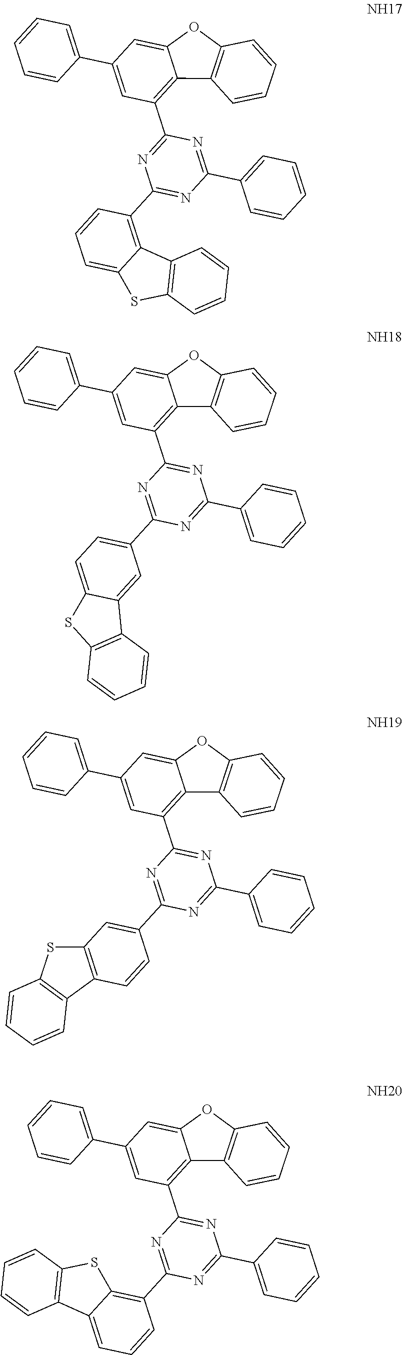

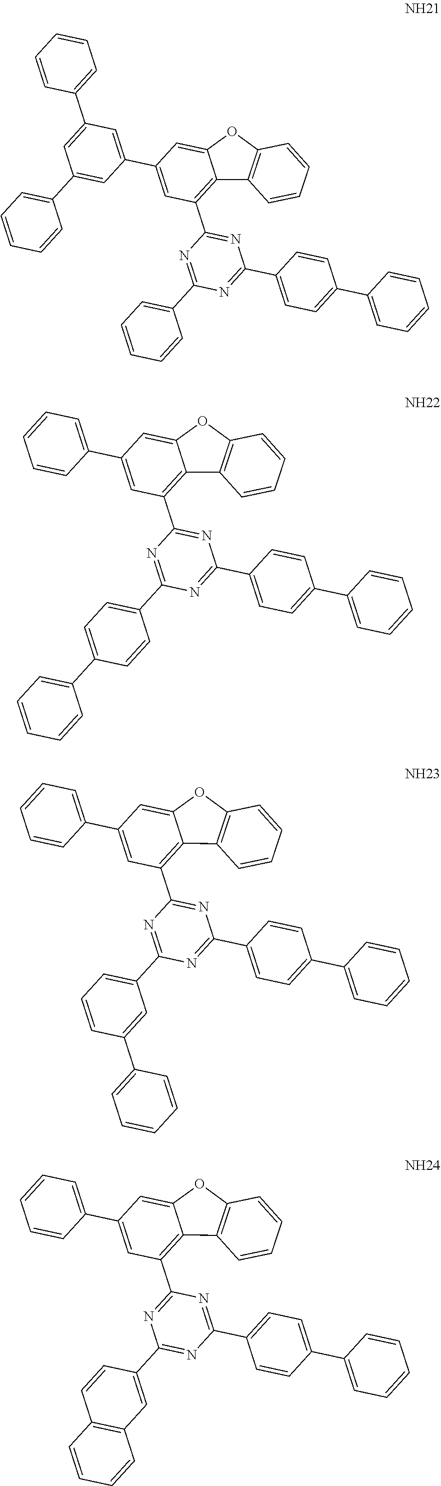

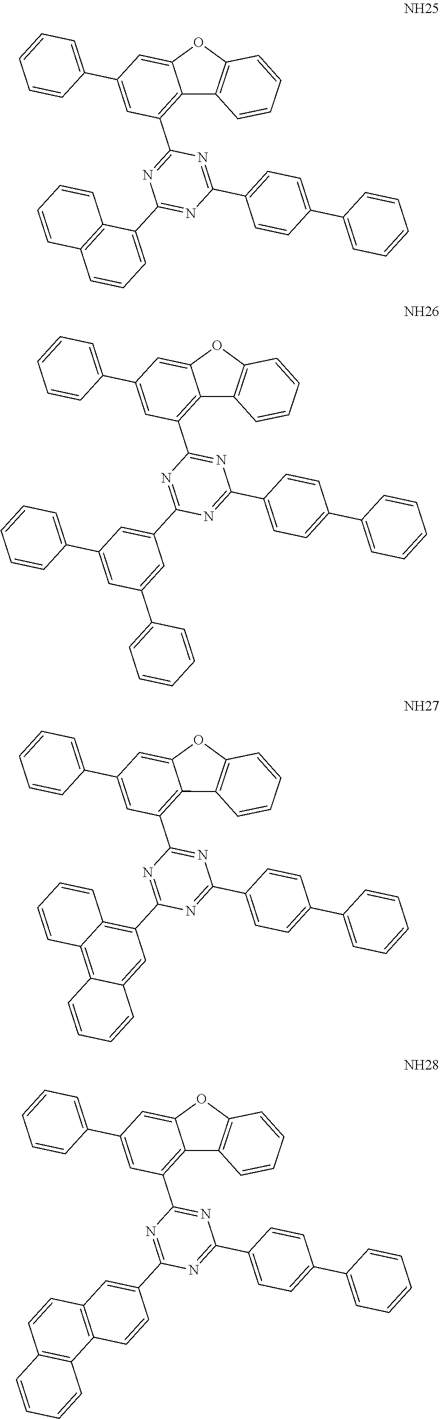 [SMILES: c1ccc(-c2ccc(-c3nc(-c4cccc5ccccc45)nc(-c4cc(-c5ccccc5)cc5oc6ccccc6c45)n3)cc2)cc1 ; c1ccc(-c2ccc(-c3nc(-c4cc(-c5ccccc5)cc(-c5ccccc5)c4)nc(-c4cc(-c5ccccc5)cc5oc6ccccc6c45)n3)cc2)cc1 ; c1ccc(-c2ccc(-c3nc(-c4cc5ccccc5c5ccccc45)nc(-c4cc(-c5ccccc5)cc5oc6ccccc6c45)n3)cc2)cc1 ; c1ccc(-c2ccc(-c3nc(-c4ccc5c(ccc6ccccc65)c4)nc(-c4cc(-c5ccccc5)cc5oc6ccccc6c45)n3)cc2)cc1]

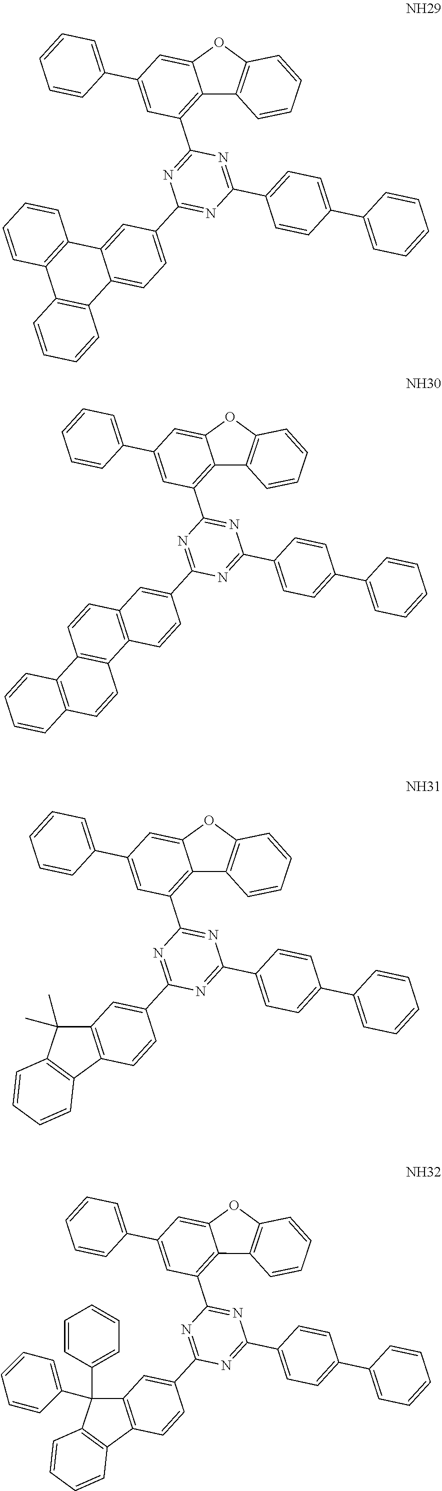 [SMILES: c1ccc(-c2ccc(-c3nc(-c4ccc5c6ccccc6c6ccccc6c5c4)nc(-c4cc(-c5ccccc5)cc5oc6ccccc6c45)n3)cc2)cc1 ; c1ccc(-c2ccc(-c3nc(-c4ccc5c(ccc6c7ccccc7ccc56)c4)nc(-c4cc(-c5ccccc5)cc5oc6ccccc6c45)n3)cc2)cc1 ; CC1(C)c2ccccc2-c2ccc(-c3nc(-c4ccc(-c5ccccc5)cc4)nc(-c4cc(-c5ccccc5)cc5oc6ccccc6c45)n3)cc21 ; c1ccc(-c2ccc(-c3nc(-c4ccc5c(c4)C(c4ccccc4)(c4ccccc4)c4ccccc4-5)nc(-c4cc(-c5ccccc5)cc5oc6ccccc6c45)n3)cc2)cc1]

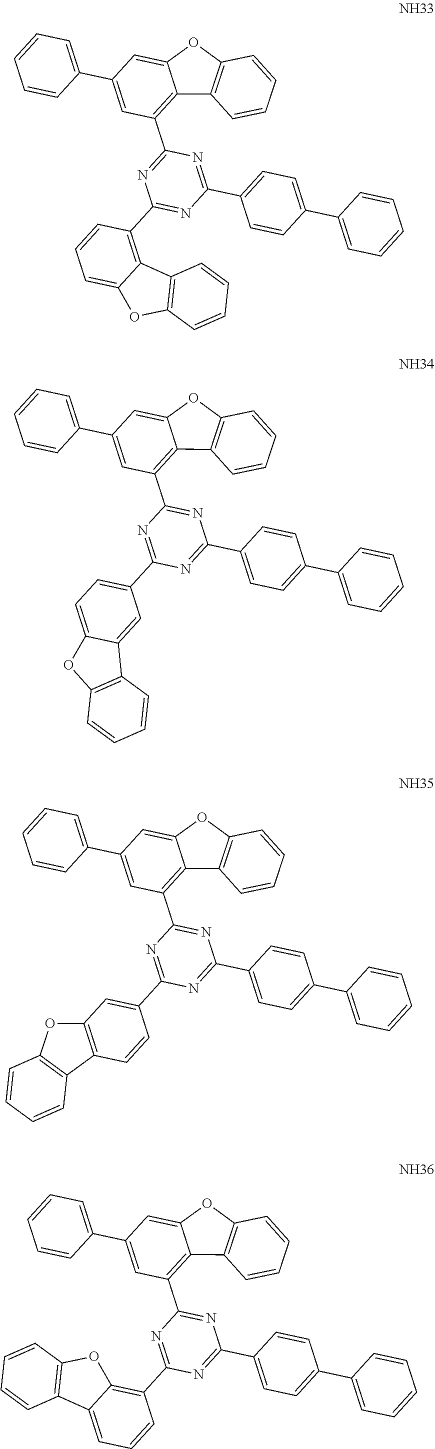 [SMILES: c1ccc(-c2ccc(-c3nc(-c4cccc5oc6ccccc6c45)nc(-c4cc(-c5ccccc5)cc5oc6ccccc6c45)n3)cc2)cc1 ; c1ccc(-c2ccc(-c3nc(-c4ccc5oc6ccccc6c5c4)nc(-c4cc(-c5ccccc5)cc5oc6ccccc6c45)n3)cc2)cc1 ; c1ccc(-c2ccc(-c3nc(-c4ccc5c(c4)oc4ccccc45)nc(-c4cc(-c5ccccc5)cc5oc6ccccc6c45)n3)cc2)cc1 ; c1ccc(-c2ccc(-c3nc(-c4cccc5c4oc4ccccc45)nc(-c4cc(-c5ccccc5)cc5oc6ccccc6c45)n3)cc2)cc1]

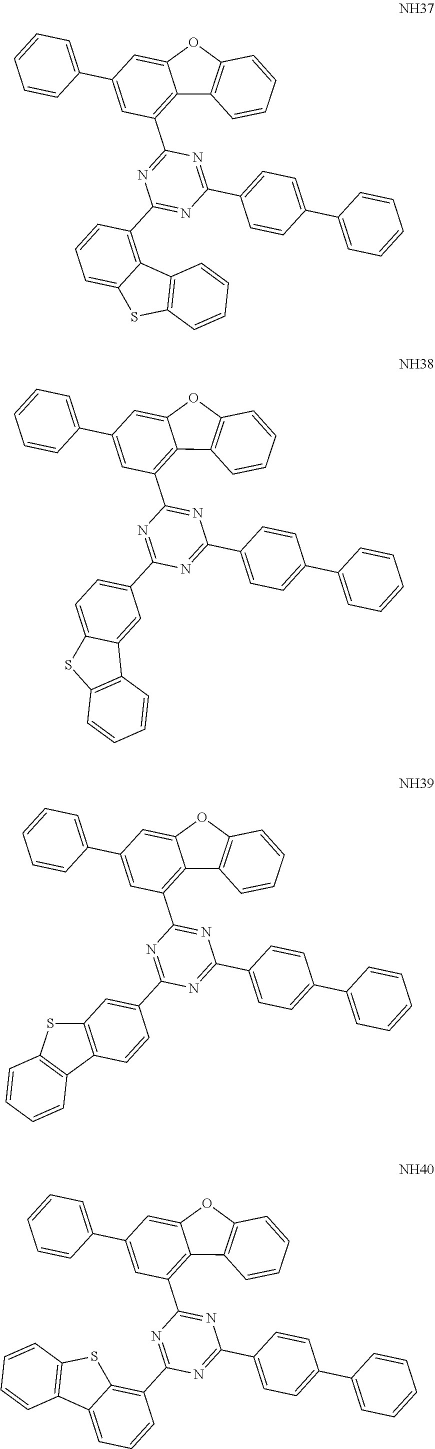 [SMILES: c1ccc(-c2ccc(-c3nc(-c4cc(-c5ccccc5)cc5oc6ccccc6c45)nc(-c4cccc5sc6ccccc6c45)n3)cc2)cc1 ; c1ccc(-c2ccc(-c3nc(-c4ccc5sc6ccccc6c5c4)nc(-c4cc(-c5ccccc5)cc5oc6ccccc6c45)n3)cc2)cc1 ; c1ccc(-c2ccc(-c3nc(-c4ccc5c(c4)sc4ccccc45)nc(-c4cc(-c5ccccc5)cc5oc6ccccc6c45)n3)cc2)cc1 ; c1ccc(-c2ccc(-c3nc(-c4cccc5c4sc4ccccc45)nc(-c4cc(-c5ccccc5)cc5oc6ccccc6c45)n3)cc2)cc1]

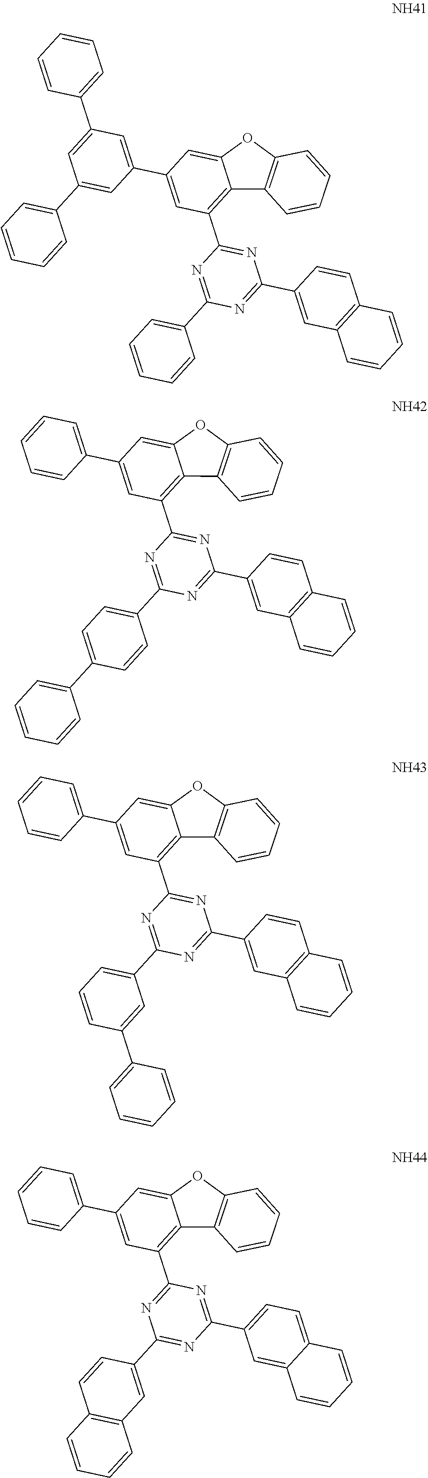

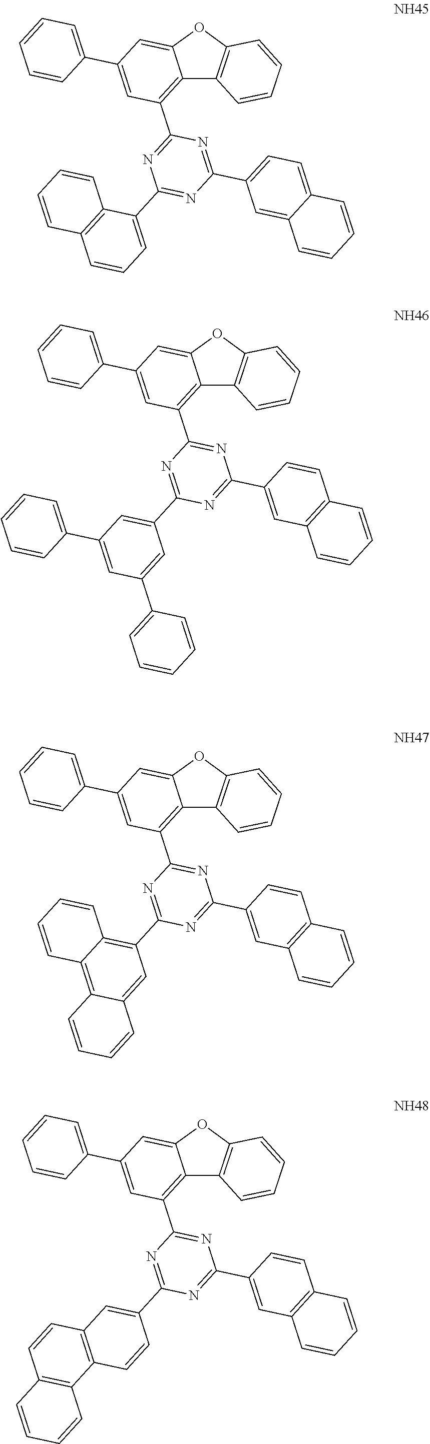 [SMILES: c1ccc(-c2cc(-c3nc(-c4ccc5ccccc5c4)nc(-c4cccc5ccccc45)n3)c3c(c2)oc2ccccc23)cc1 ; c1ccc(-c2cc(-c3ccccc3)cc(-c3nc(-c4ccc5ccccc5c4)nc(-c4cc(-c5ccccc5)cc5oc6ccccc6c45)n3)c2)cc1 ; c1ccc(-c2cc(-c3nc(-c4ccc5ccccc5c4)nc(-c4cc5ccccc5c5ccccc45)n3)c3c(c2)oc2ccccc23)cc1 ; c1ccc(-c2cc(-c3nc(-c4ccc5ccccc5c4)nc(-c4ccc5c(ccc6ccccc65)c4)n3)c3c(c2)oc2ccccc23)cc1]

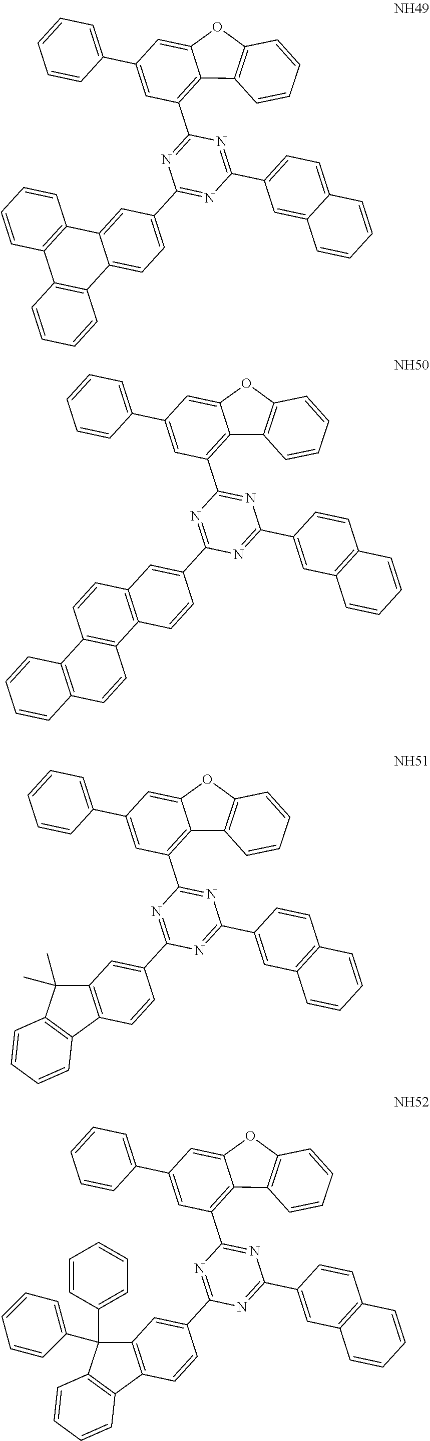

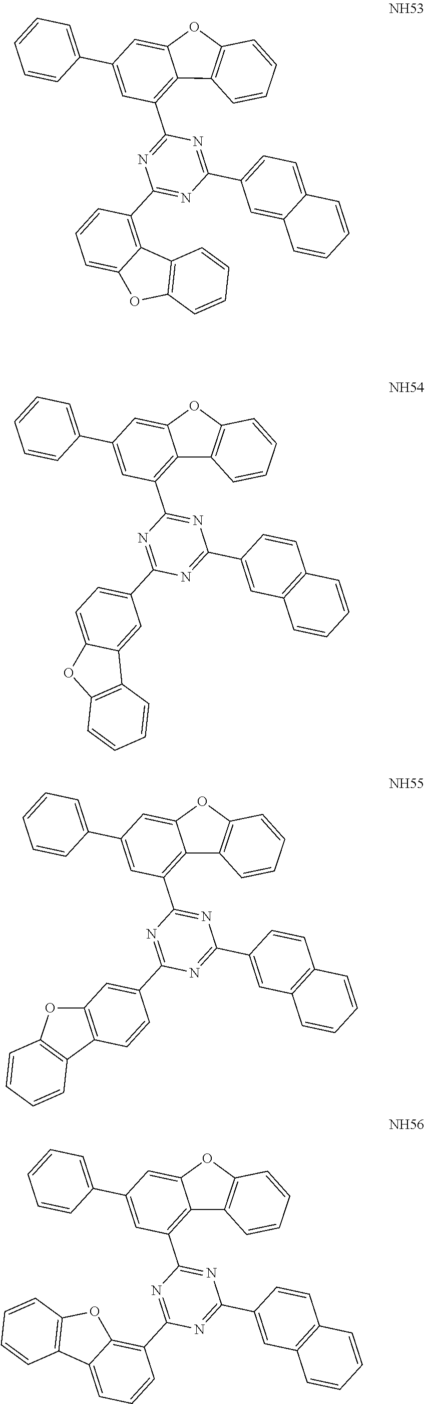 [SMILES: c1ccc(-c2cc(-c3nc(-c4ccc5ccccc5c4)nc(-c4cccc5oc6ccccc6c45)n3)c3c(c2)oc2ccccc23)cc1 ; c1ccc(-c2cc(-c3nc(-c4ccc5ccccc5c4)nc(-c4ccc5oc6ccccc6c5c4)n3)c3c(c2)oc2ccccc23)cc1 ; c1ccc(-c2cc(-c3nc(-c4ccc5ccccc5c4)nc(-c4ccc5c(c4)oc4ccccc45)n3)c3c(c2)oc2ccccc23)cc1 ; c1ccc(-c2cc(-c3nc(-c4ccc5ccccc5c4)nc(-c4cccc5c4oc4ccccc45)n3)c3c(c2)oc2ccccc23)cc1]

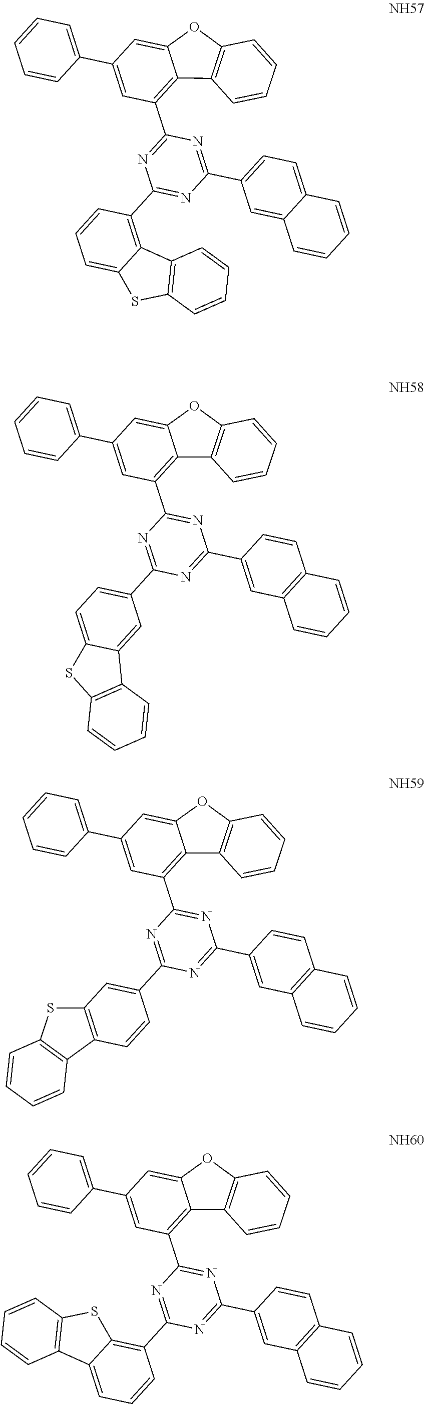 [SMILES: c1ccc(-c2cc(-c3nc(-c4ccc5ccccc5c4)nc(-c4cccc5sc6ccccc6c45)n3)c3c(c2)oc2ccccc23)cc1 ; c1ccc(-c2cc(-c3nc(-c4ccc5ccccc5c4)nc(-c4ccc5sc6ccccc6c5c4)n3)c3c(c2)oc2ccccc23)cc1 ; c1ccc(-c2cc(-c3nc(-c4ccc5ccccc5c4)nc(-c4ccc5c(c4)sc4ccccc45)n3)c3c(c2)oc2ccccc23)cc1 ; c1ccc(-c2cc(-c3nc(-c4ccc5ccccc5c4)nc(-c4cccc5c4sc4ccccc45)n3)c3c(c2)oc2ccccc23)cc1]

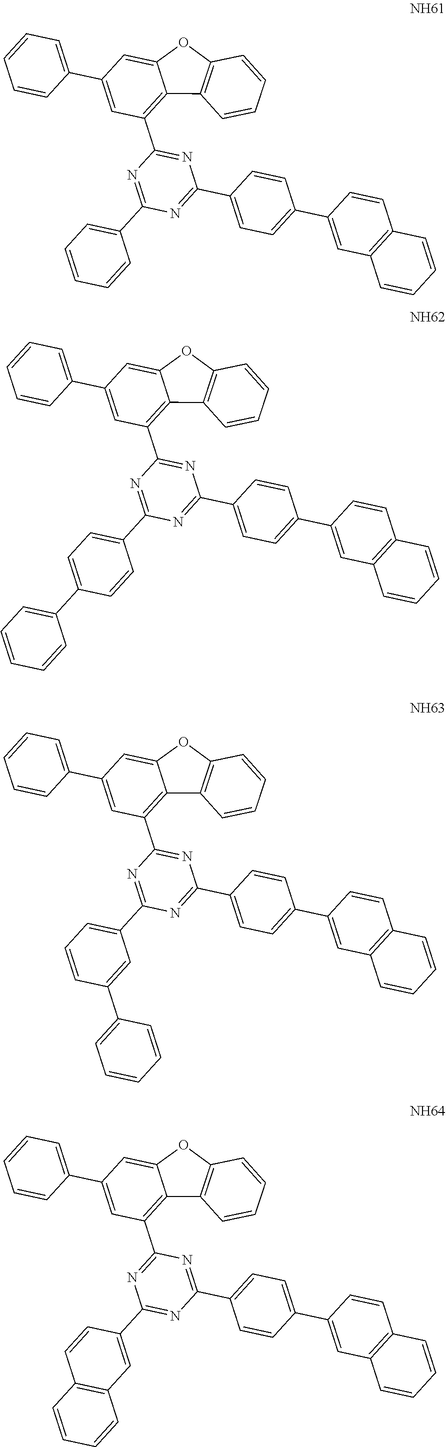 [SMILES: c1ccc(-c2cc(-c3nc(-c4ccccc4)nc(-c4ccc(-c5ccc6ccccc6c5)cc4)n3)c3c(c2)oc2ccccc23)cc1 ; c1ccc(-c2ccc(-c3nc(-c4ccc(-c5ccc6ccccc6c5)cc4)nc(-c4cc(-c5ccccc5)cc5oc6ccccc6c45)n3)cc2)cc1 ; c1ccc(-c2cccc(-c3nc(-c4ccc(-c5ccc6ccccc6c5)cc4)nc(-c4cc(-c5ccccc5)cc5oc6ccccc6c45)n3)c2)cc1 ; c1ccc(-c2cc(-c3nc(-c4ccc(-c5ccc6ccccc6c5)cc4)nc(-c4ccc5ccccc5c4)n3)c3c(c2)oc2ccccc23)cc1]

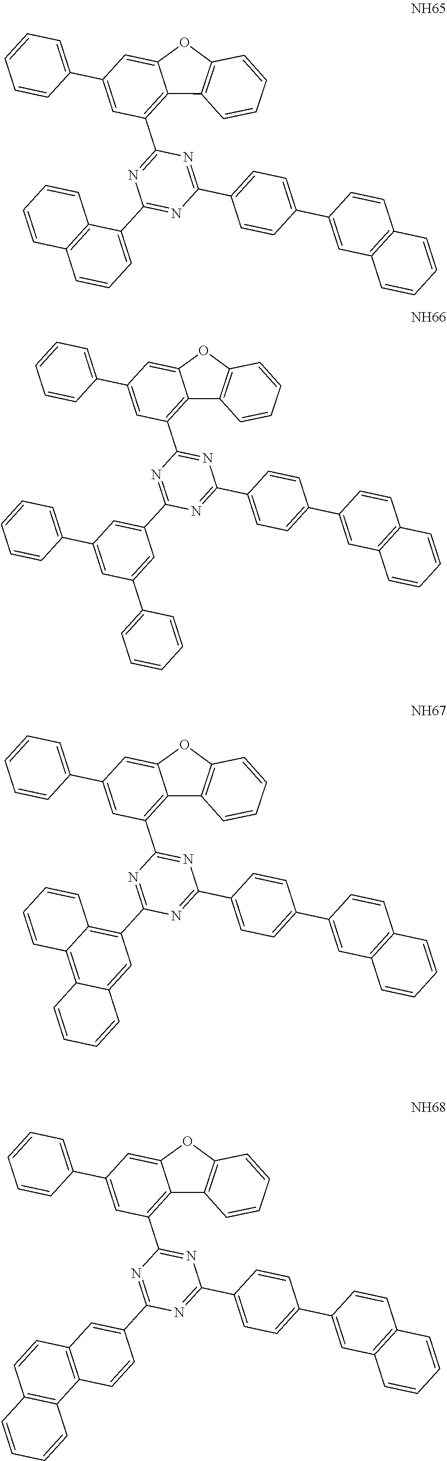 [SMILES: c1ccc(-c2cc(-c3nc(-c4ccc(-c5ccc6ccccc6c5)cc4)nc(-c4cccc5ccccc45)n3)c3c(c2)oc2ccccc23)cc1 ; c1ccc(-c2cc(-c3ccccc3)cc(-c3nc(-c4ccc(-c5ccc6ccccc6c5)cc4)nc(-c4cc(-c5ccccc5)cc5oc6ccccc6c45)n3)c2)cc1 ; c1ccc(-c2cc(-c3nc(-c4ccc(-c5ccc6ccccc6c5)cc4)nc(-c4cc5ccccc5c5ccccc45)n3)c3c(c2)oc2ccccc23)cc1 ; c1ccc(-c2cc(-c3nc(-c4ccc(-c5ccc6ccccc6c5)cc4)nc(-c4ccc5c(ccc6ccccc65)c4)n3)c3c(c2)oc2ccccc23)cc1]

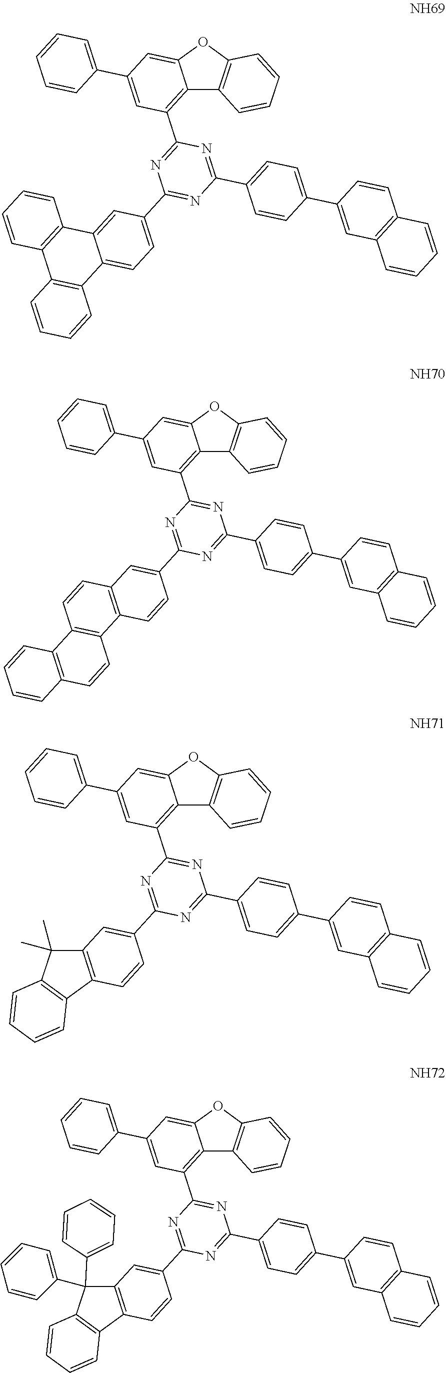 [SMILES: c1ccc(-c2cc(-c3nc(-c4ccc(-c5ccc6ccccc6c5)cc4)nc(-c4ccc5c6ccccc6c6ccccc6c5c4)n3)c3c(c2)oc2ccccc23)cc1 ; c1ccc(-c2cc(-c3nc(-c4ccc(-c5ccc6ccccc6c5)cc4)nc(-c4ccc5c(ccc6c7ccccc7ccc56)c4)n3)c3c(c2)oc2ccccc23)cc1 ; CC1(C)c2ccccc2-c2ccc(-c3nc(-c4ccc(-c5ccc6ccccc6c5)cc4)nc(-c4cc(-c5ccccc5)cc5oc6ccccc6c45)n3)cc21 ; c1ccc(-c2cc(-c3nc(-c4ccc(-c5ccc6ccccc6c5)cc4)nc(-c4ccc5c(c4)C(c4ccccc4)(c4ccccc4)c4ccccc4-5)n3)c3c(c2)oc2ccccc23)cc1]

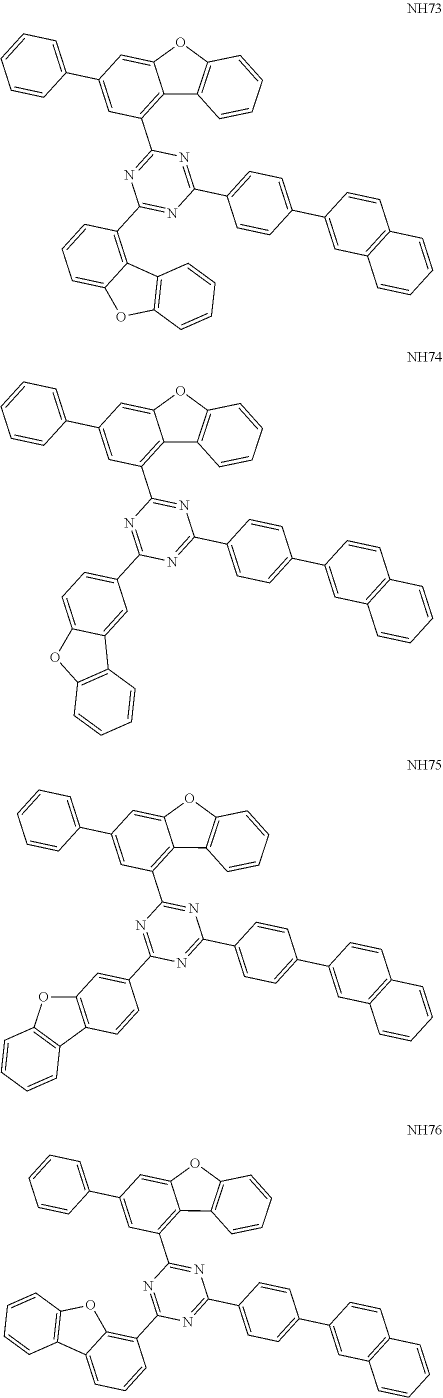 [SMILES: c1ccc(-c2cc(-c3nc(-c4ccc(-c5ccc6ccccc6c5)cc4)nc(-c4cccc5oc6ccccc6c45)n3)c3c(c2)oc2ccccc23)cc1 ; c1ccc(-c2cc(-c3nc(-c4ccc(-c5ccc6ccccc6c5)cc4)nc(-c4ccc5oc6ccccc6c5c4)n3)c3c(c2)oc2ccccc23)cc1 ; c1ccc(-c2cc(-c3nc(-c4ccc(-c5ccc6ccccc6c5)cc4)nc(-c4ccc5c(c4)oc4ccccc45)n3)c3c(c2)oc2ccccc23)cc1 ; c1ccc(-c2cc(-c3nc(-c4ccc(-c5ccc6ccccc6c5)cc4)nc(-c4cccc5c4oc4ccccc45)n3)c3c(c2)oc2ccccc23)cc1]

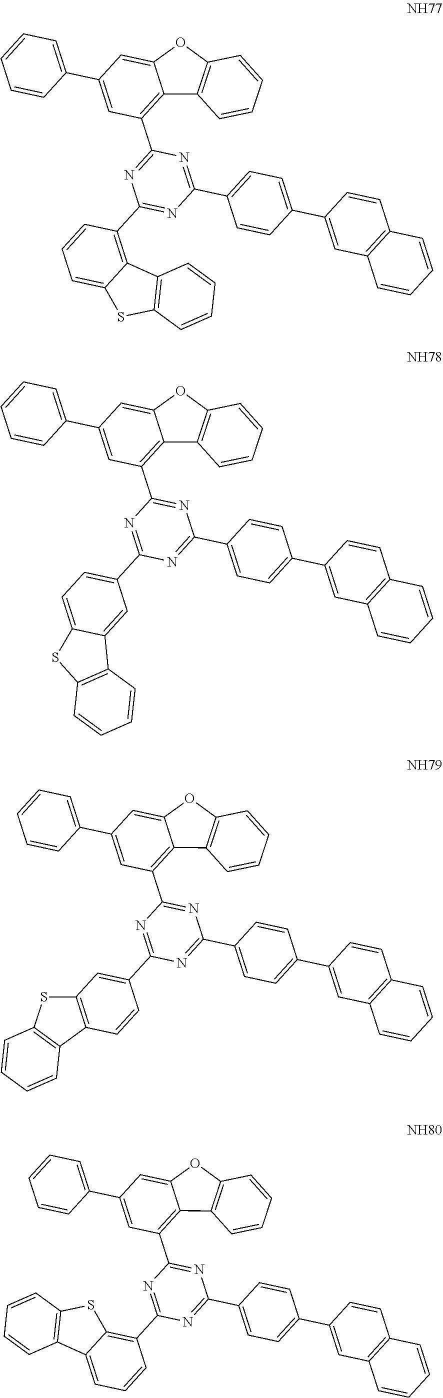 [SMILES: c1ccc(-c2cc(-c3nc(-c4ccc(-c5ccc6ccccc6c5)cc4)nc(-c4cccc5sc6ccccc6c45)n3)c3c(c2)oc2ccccc23)cc1 ; c1ccc(-c2cc(-c3nc(-c4ccc(-c5ccc6ccccc6c5)cc4)nc(-c4ccc5sc6ccccc6c5c4)n3)c3c(c2)oc2ccccc23)cc1 ; c1ccc(-c2cc(-c3nc(-c4ccc(-c5ccc6ccccc6c5)cc4)nc(-c4ccc5c(c4)sc4ccccc45)n3)c3c(c2)oc2ccccc23)cc1 ; c1ccc(-c2cc(-c3nc(-c4ccc(-c5ccc6ccccc6c5)cc4)nc(-c4cccc5c4sc4ccccc45)n3)c3c(c2)oc2ccccc23)cc1]

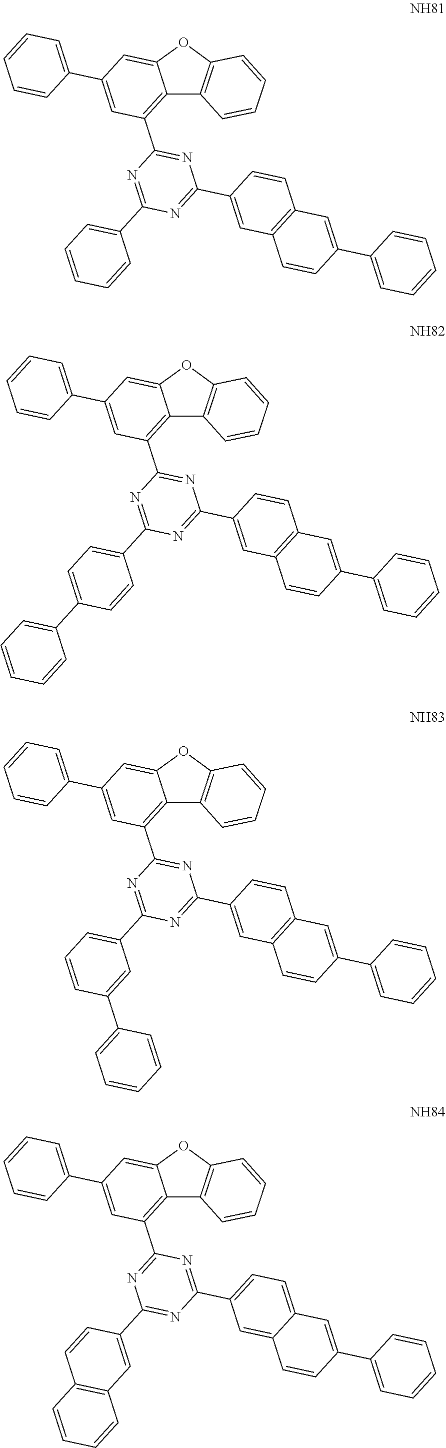 [SMILES: c1ccc(-c2ccc3cc(-c4nc(-c5ccccc5)nc(-c5cc(-c6ccccc6)cc6oc7ccccc7c56)n4)ccc3c2)cc1 ; c1ccc(-c2ccc(-c3nc(-c4ccc5cc(-c6ccccc6)ccc5c4)nc(-c4cc(-c5ccccc5)cc5oc6ccccc6c45)n3)cc2)cc1 ; c1ccc(-c2cccc(-c3nc(-c4ccc5cc(-c6ccccc6)ccc5c4)nc(-c4cc(-c5ccccc5)cc5oc6ccccc6c45)n3)c2)cc1 ; c1ccc(-c2ccc3cc(-c4nc(-c5ccc6ccccc6c5)nc(-c5cc(-c6ccccc6)cc6oc7ccccc7c56)n4)ccc3c2)cc1]

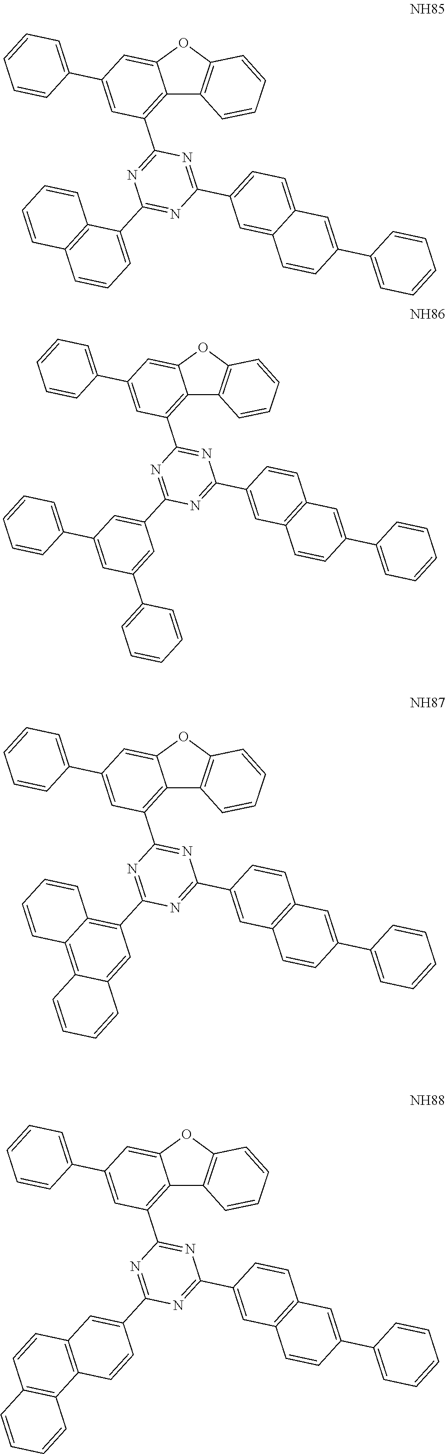 [SMILES: c1ccc(-c2ccc3cc(-c4nc(-c5cccc6ccccc56)nc(-c5cc(-c6ccccc6)cc6oc7ccccc7c56)n4)ccc3c2)cc1 ; c1ccc(-c2cc(-c3ccccc3)cc(-c3nc(-c4ccc5cc(-c6ccccc6)ccc5c4)nc(-c4cc(-c5ccccc5)cc5oc6ccccc6c45)n3)c2)cc1 ; c1ccc(-c2ccc3cc(-c4nc(-c5cc6ccccc6c6ccccc56)nc(-c5cc(-c6ccccc6)cc6oc7ccccc7c56)n4)ccc3c2)cc1 ; c1ccc(-c2ccc3cc(-c4nc(-c5ccc6c(ccc7ccccc76)c5)nc(-c5cc(-c6ccccc6)cc6oc7ccccc7c56)n4)ccc3c2)cc1]

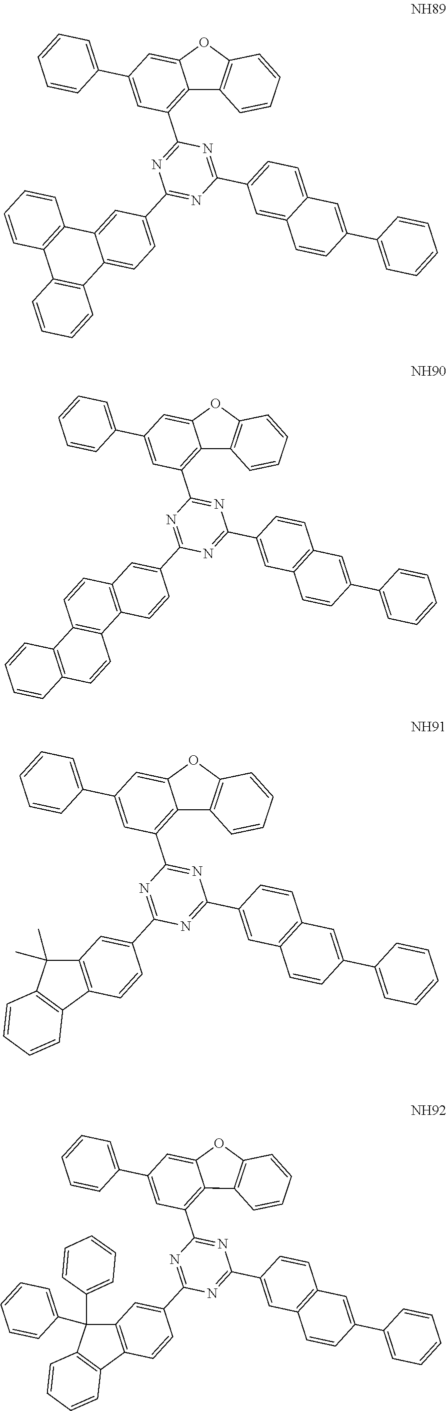 [SMILES: c1ccc(-c2ccc3cc(-c4nc(-c5ccc6c7ccccc7c7ccccc7c6c5)nc(-c5cc(-c6ccccc6)cc6oc7ccccc7c56)n4)ccc3c2)cc1 ; c1ccc(-c2ccc3cc(-c4nc(-c5ccc6c(ccc7c8ccccc8ccc67)c5)nc(-c5cc(-c6ccccc6)cc6oc7ccccc7c56)n4)ccc3c2)cc1 ; CC1(C)c2ccccc2-c2ccc(-c3nc(-c4ccc5cc(-c6ccccc6)ccc5c4)nc(-c4cc(-c5ccccc5)cc5oc6ccccc6c45)n3)cc21 ; c1ccc(-c2ccc3cc(-c4nc(-c5ccc6c(c5)C(c5ccccc5)(c5ccccc5)c5ccccc5-6)nc(-c5cc(-c6ccccc6)cc6oc7ccccc7c56)n4)ccc3c2)cc1]

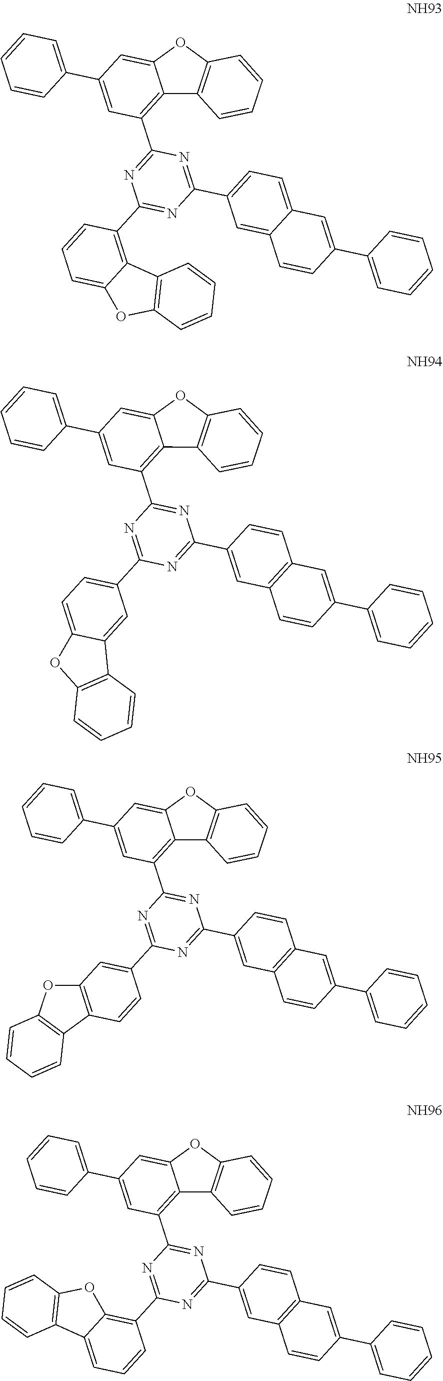 [SMILES: c1ccc(-c2ccc3cc(-c4nc(-c5cccc6oc7ccccc7c56)nc(-c5cc(-c6ccccc6)cc6oc7ccccc7c56)n4)ccc3c2)cc1 ; c1ccc(-c2ccc3cc(-c4nc(-c5ccc6oc7ccccc7c6c5)nc(-c5cc(-c6ccccc6)cc6oc7ccccc7c56)n4)ccc3c2)cc1 ; c1ccc(-c2ccc3cc(-c4nc(-c5ccc6c(c5)oc5ccccc56)nc(-c5cc(-c6ccccc6)cc6oc7ccccc7c56)n4)ccc3c2)cc1 ; c1ccc(-c2ccc3cc(-c4nc(-c5cccc6c5oc5ccccc56)nc(-c5cc(-c6ccccc6)cc6oc7ccccc7c56)n4)ccc3c2)cc1]

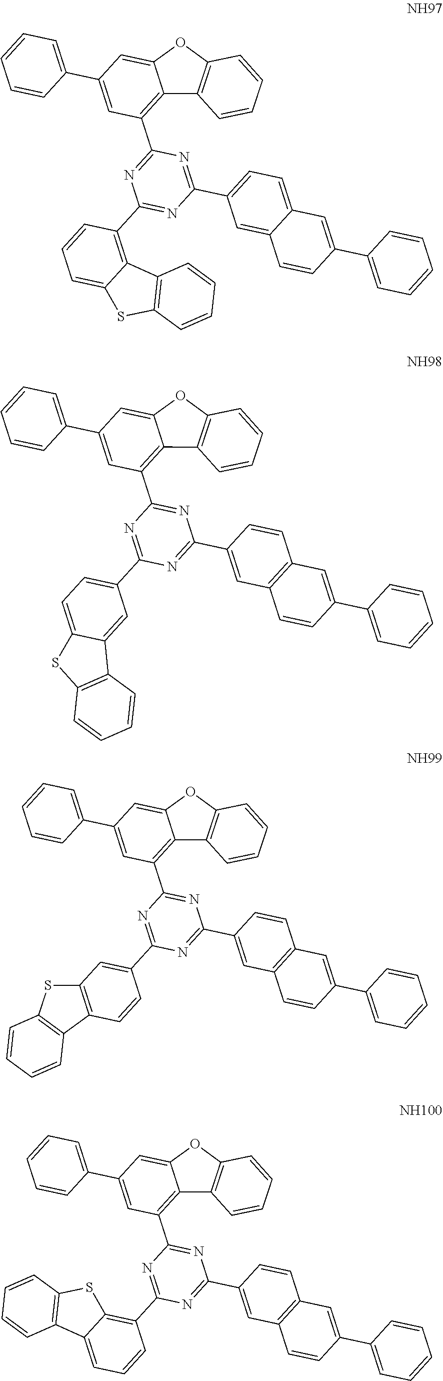 [SMILES: c1ccc(-c2ccc3cc(-c4nc(-c5cc(-c6ccccc6)cc6oc7ccccc7c56)nc(-c5cccc6sc7ccccc7c56)n4)ccc3c2)cc1 ; c1ccc(-c2ccc3cc(-c4nc(-c5ccc6sc7ccccc7c6c5)nc(-c5cc(-c6ccccc6)cc6oc7ccccc7c56)n4)ccc3c2)cc1 ; c1ccc(-c2ccc3cc(-c4nc(-c5ccc6c(c5)sc5ccccc56)nc(-c5cc(-c6ccccc6)cc6oc7ccccc7c56)n4)ccc3c2)cc1 ; c1ccc(-c2ccc3cc(-c4nc(-c5cccc6c5sc5ccccc56)nc(-c5cc(-c6ccccc6)cc6oc7ccccc7c56)n4)ccc3c2)cc1]

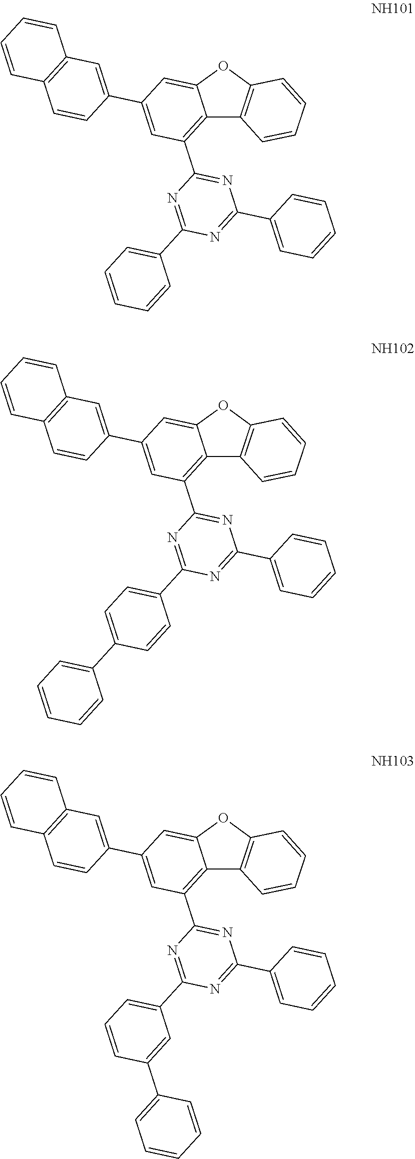 [SMILES: c1ccc(-c2nc(-c3ccccc3)nc(-c3cc(-c4ccc5ccccc5c4)cc4oc5ccccc5c34)n2)cc1 ; c1ccc(-c2ccc(-c3nc(-c4ccccc4)nc(-c4cc(-c5ccc6ccccc6c5)cc5oc6ccccc6c45)n3)cc2)cc1 ; c1ccc(-c2cccc(-c3nc(-c4ccccc4)nc(-c4cc(-c5ccc6ccccc6c5)cc5oc6ccccc6c45)n3)c2)cc1]

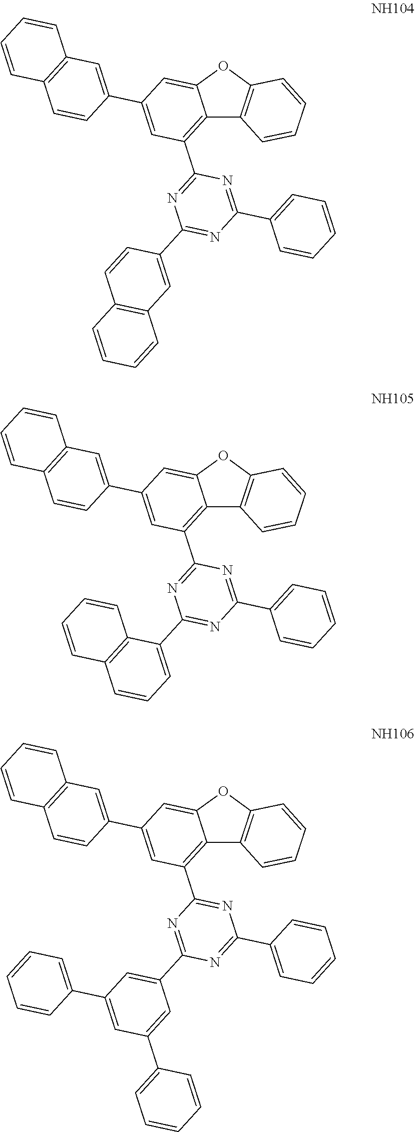 [SMILES: c1ccc(-c2nc(-c3ccc4ccccc4c3)nc(-c3cc(-c4ccc5ccccc5c4)cc4oc5ccccc5c34)n2)cc1 ; c1ccc(-c2nc(-c3cccc4ccccc34)nc(-c3cc(-c4ccc5ccccc5c4)cc4oc5ccccc5c34)n2)cc1 ; c1ccc(-c2cc(-c3ccccc3)cc(-c3nc(-c4ccccc4)nc(-c4cc(-c5ccc6ccccc6c5)cc5oc6ccccc6c45)n3)c2)cc1]

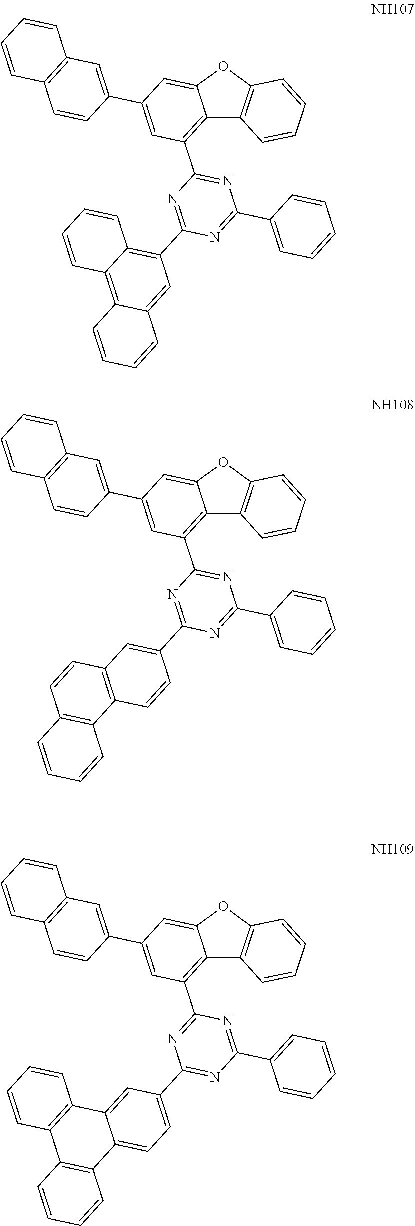 [SMILES: c1ccc(-c2nc(-c3cc4ccccc4c4ccccc34)nc(-c3cc(-c4ccc5ccccc5c4)cc4oc5ccccc5c34)n2)cc1 ; c1ccc(-c2nc(-c3ccc4c(ccc5ccccc54)c3)nc(-c3cc(-c4ccc5ccccc5c4)cc4oc5ccccc5c34)n2)cc1 ; c1ccc(-c2nc(-c3ccc4c5ccccc5c5ccccc5c4c3)nc(-c3cc(-c4ccc5ccccc5c4)cc4oc5ccccc5c34)n2)cc1]

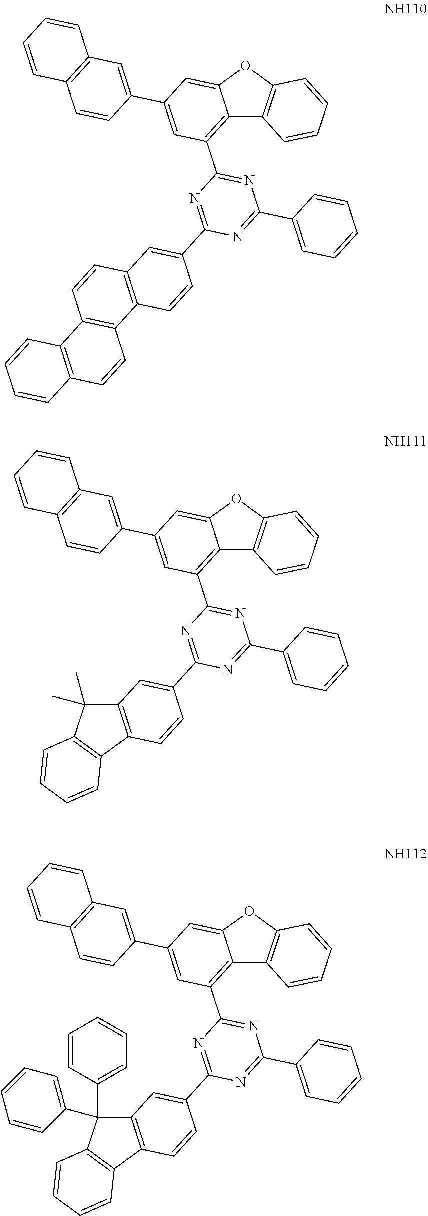 [SMILES: c1ccc(-c2nc(-c3ccc4c(ccc5c6ccccc6ccc45)c3)nc(-c3cc(-c4ccc5ccccc5c4)cc4oc5ccccc5c34)n2)cc1 ; CC1(C)c2ccccc2-c2ccc(-c3nc(-c4ccccc4)nc(-c4cc(-c5ccc6ccccc6c5)cc5oc6ccccc6c45)n3)cc21 ; c1ccc(-c2nc(-c3ccc4c(c3)C(c3ccccc3)(c3ccccc3)c3ccccc3-4)nc(-c3cc(-c4ccc5ccccc5c4)cc4oc5ccccc5c34)n2)cc1]

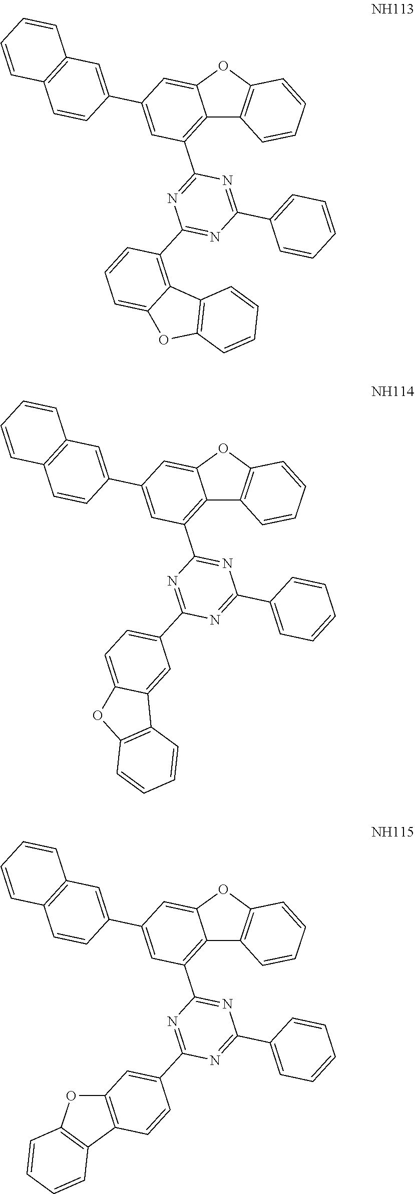 [SMILES: c1ccc(-c2nc(-c3cccc4oc5ccccc5c34)nc(-c3cc(-c4ccc5ccccc5c4)cc4oc5ccccc5c34)n2)cc1 ; c1ccc(-c2nc(-c3ccc4oc5ccccc5c4c3)nc(-c3cc(-c4ccc5ccccc5c4)cc4oc5ccccc5c34)n2)cc1 ; c1ccc(-c2nc(-c3ccc4c(c3)oc3ccccc34)nc(-c3cc(-c4ccc5ccccc5c4)cc4oc5ccccc5c34)n2)cc1]

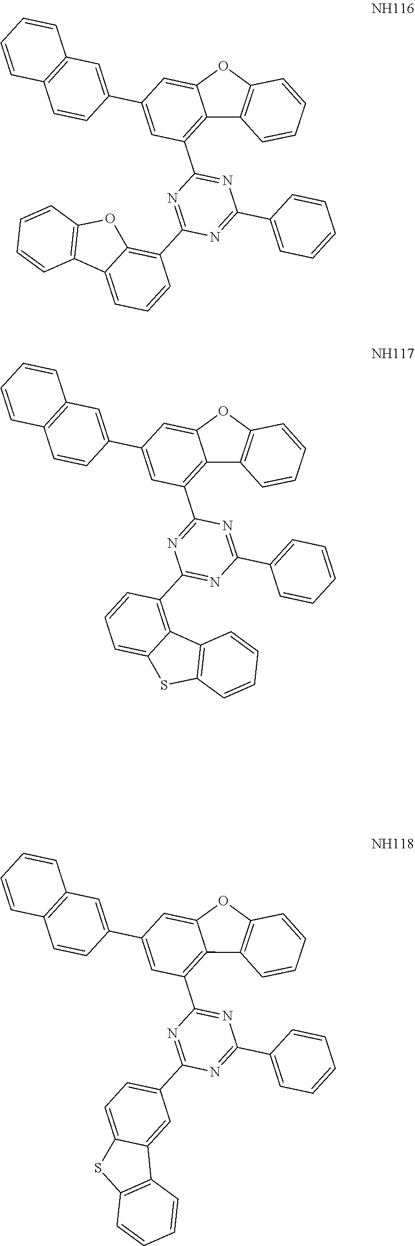 [SMILES: c1ccc(-c2nc(-c3cccc4c3oc3ccccc34)nc(-c3cc(-c4ccc5ccccc5c4)cc4oc5ccccc5c34)n2)cc1 ; c1ccc(-c2nc(-c3cc(-c4ccc5ccccc5c4)cc4oc5ccccc5c34)nc(-c3cccc4sc5ccccc5c34)n2)cc1 ; c1ccc(-c2nc(-c3ccc4sc5ccccc5c4c3)nc(-c3cc(-c4ccc5ccccc5c4)cc4oc5ccccc5c34)n2)cc1]

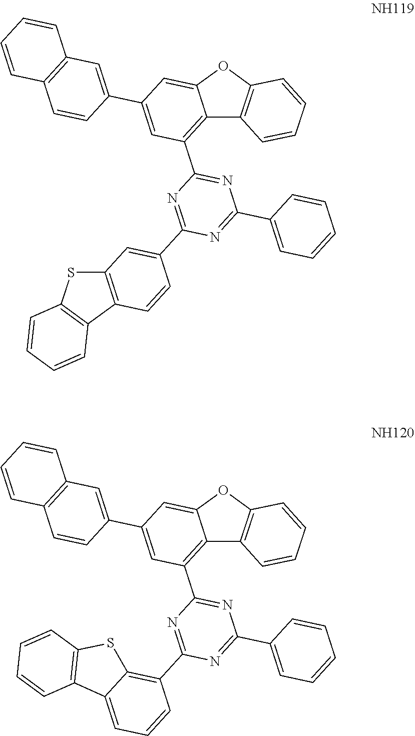 [SMILES: c1ccc(-c2nc(-c3ccc4c(c3)sc3ccccc34)nc(-c3cc(-c4ccc5ccccc5c4)cc4oc5ccccc5c34)n2)cc1 ; c1ccc(-c2nc(-c3cccc4c3sc3ccccc34)nc(-c3cc(-c4ccc5ccccc5c4)cc4oc5ccccc5c34)n2)cc1]

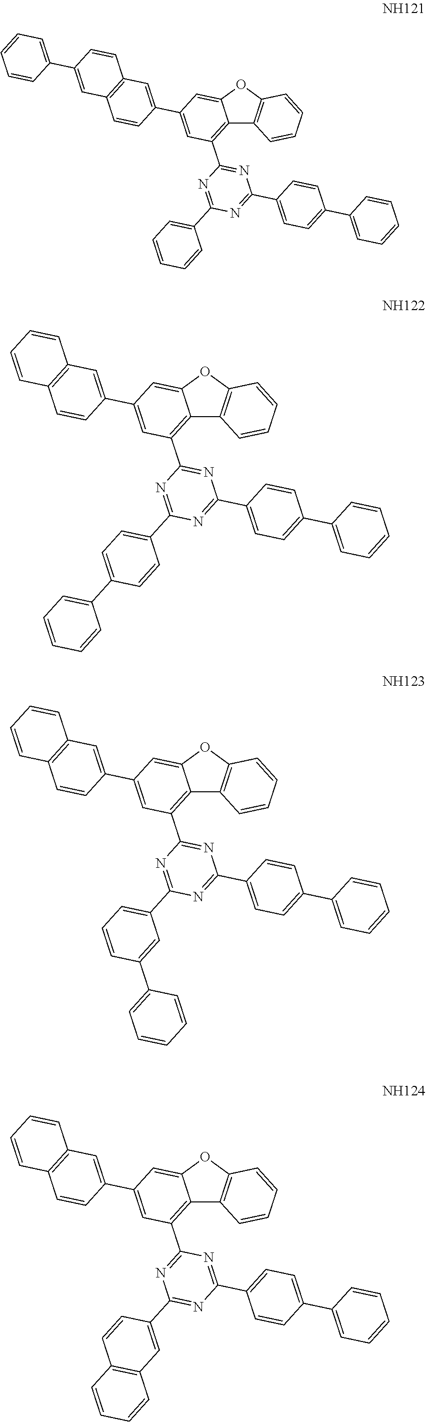 [SMILES: c1ccc(-c2ccc(-c3nc(-c4ccccc4)nc(-c4cc(-c5ccc6cc(-c7ccccc7)ccc6c5)cc5oc6ccccc6c45)n3)cc2)cc1 ; c1ccc(-c2ccc(-c3nc(-c4ccc(-c5ccccc5)cc4)nc(-c4cc(-c5ccc6ccccc6c5)cc5oc6ccccc6c45)n3)cc2)cc1 ; c1ccc(-c2ccc(-c3nc(-c4cccc(-c5ccccc5)c4)nc(-c4cc(-c5ccc6ccccc6c5)cc5oc6ccccc6c45)n3)cc2)cc1 ; c1ccc(-c2ccc(-c3nc(-c4ccc5ccccc5c4)nc(-c4cc(-c5ccc6ccccc6c5)cc5oc6ccccc6c45)n3)cc2)cc1]

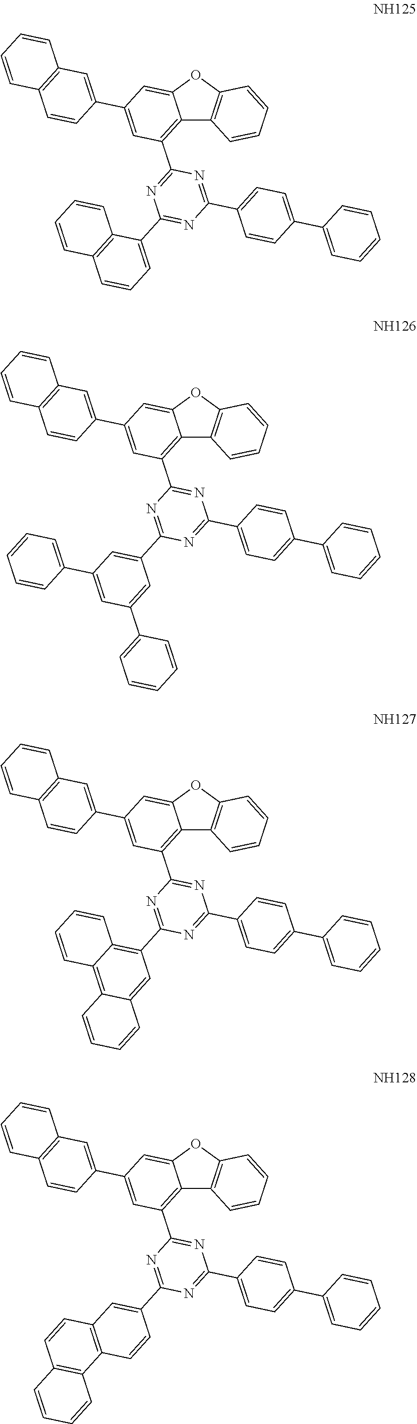 [SMILES: c1ccc(-c2ccc(-c3nc(-c4cccc5ccccc45)nc(-c4cc(-c5ccc6ccccc6c5)cc5oc6ccccc6c45)n3)cc2)cc1 ; c1ccc(-c2ccc(-c3nc(-c4cc(-c5ccccc5)cc(-c5ccccc5)c4)nc(-c4cc(-c5ccc6ccccc6c5)cc5oc6ccccc6c45)n3)cc2)cc1 ; c1ccc(-c2ccc(-c3nc(-c4cc5ccccc5c5ccccc45)nc(-c4cc(-c5ccc6ccccc6c5)cc5oc6ccccc6c45)n3)cc2)cc1 ; c1ccc(-c2ccc(-c3nc(-c4ccc5c(ccc6ccccc65)c4)nc(-c4cc(-c5ccc6ccccc6c5)cc5oc6ccccc6c45)n3)cc2)cc1]

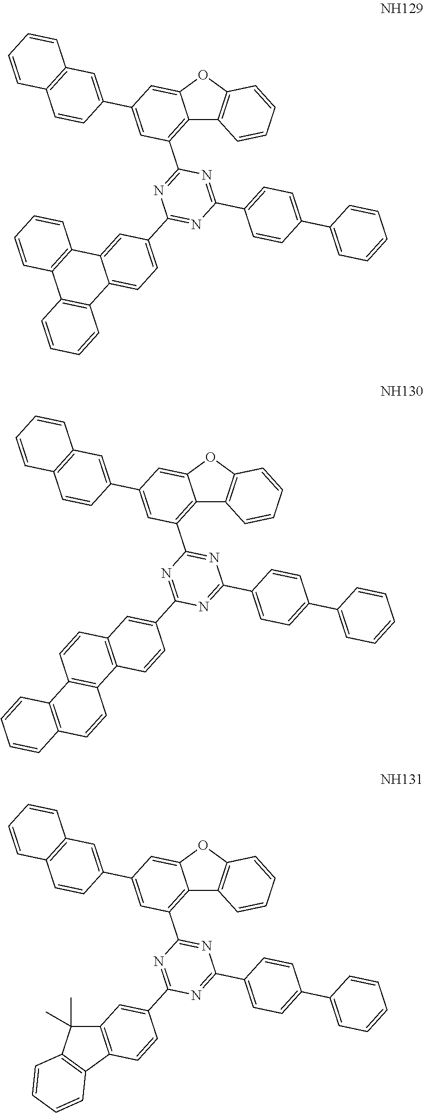 [SMILES: c1ccc(-c2ccc(-c3nc(-c4ccc5c6ccccc6c6ccccc6c5c4)nc(-c4cc(-c5ccc6ccccc6c5)cc5oc6ccccc6c45)n3)cc2)cc1 ; c1ccc(-c2ccc(-c3nc(-c4ccc5c(ccc6c7ccccc7ccc56)c4)nc(-c4cc(-c5ccc6ccccc6c5)cc5oc6ccccc6c45)n3)cc2)cc1 ; CC1(C)c2ccccc2-c2ccc(-c3nc(-c4ccc(-c5ccccc5)cc4)nc(-c4cc(-c5ccc6ccccc6c5)cc5oc6ccccc6c45)n3)cc21]

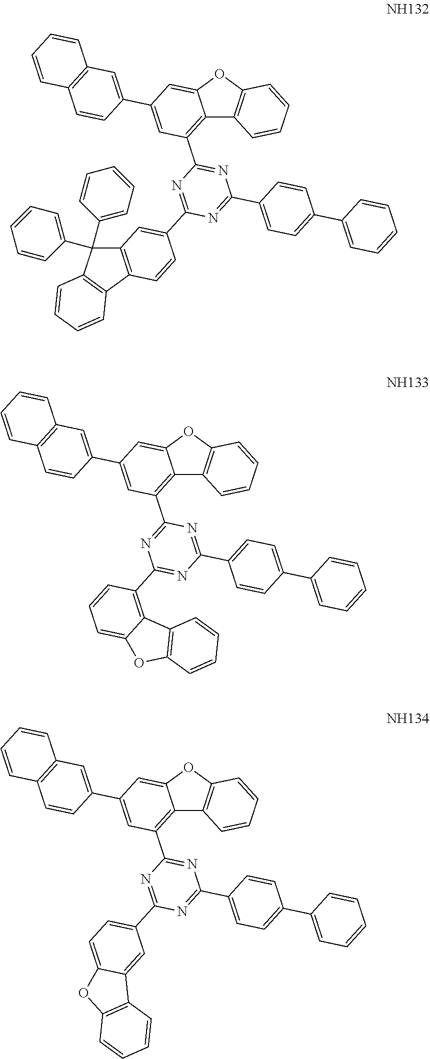 [SMILES: c1ccc(-c2ccc(-c3nc(-c4ccc5c(c4)C(c4ccccc4)(c4ccccc4)c4ccccc4-5)nc(-c4cc(-c5ccc6ccccc6c5)cc5oc6ccccc6c45)n3)cc2)cc1 ; c1ccc(-c2ccc(-c3nc(-c4cccc5oc6ccccc6c45)nc(-c4cc(-c5ccc6ccccc6c5)cc5oc6ccccc6c45)n3)cc2)cc1 ; c1ccc(-c2ccc(-c3nc(-c4ccc5oc6ccccc6c5c4)nc(-c4cc(-c5ccc6ccccc6c5)cc5oc6ccccc6c45)n3)cc2)cc1]

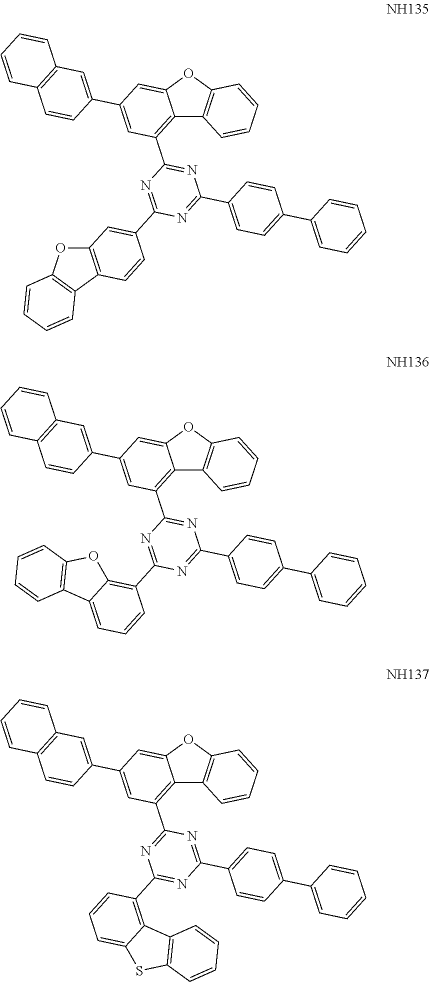 [SMILES: c1ccc(-c2ccc(-c3nc(-c4ccc5c(c4)oc4ccccc45)nc(-c4cc(-c5ccc6ccccc6c5)cc5oc6ccccc6c45)n3)cc2)cc1 ; c1ccc(-c2ccc(-c3nc(-c4cccc5c4oc4ccccc45)nc(-c4cc(-c5ccc6ccccc6c5)cc5oc6ccccc6c45)n3)cc2)cc1 ; c1ccc(-c2ccc(-c3nc(-c4cc(-c5ccc6ccccc6c5)cc5oc6ccccc6c45)nc(-c4cccc5sc6ccccc6c45)n3)cc2)cc1]

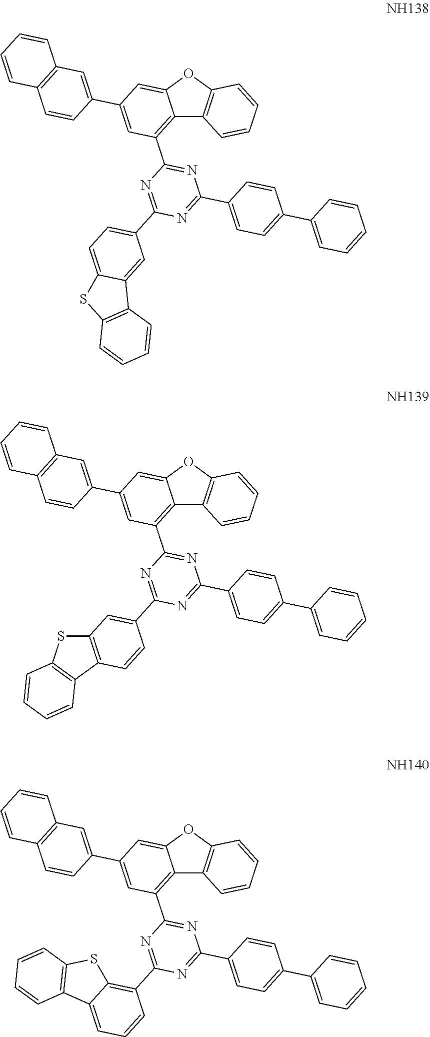 [SMILES: c1ccc(-c2ccc(-c3nc(-c4ccc5sc6ccccc6c5c4)nc(-c4cc(-c5ccc6ccccc6c5)cc5oc6ccccc6c45)n3)cc2)cc1 ; c1ccc(-c2ccc(-c3nc(-c4ccc5c(c4)sc4ccccc45)nc(-c4cc(-c5ccc6ccccc6c5)cc5oc6ccccc6c45)n3)cc2)cc1 ; c1ccc(-c2ccc(-c3nc(-c4cccc5c4sc4ccccc45)nc(-c4cc(-c5ccc6ccccc6c5)cc5oc6ccccc6c45)n3)cc2)cc1]

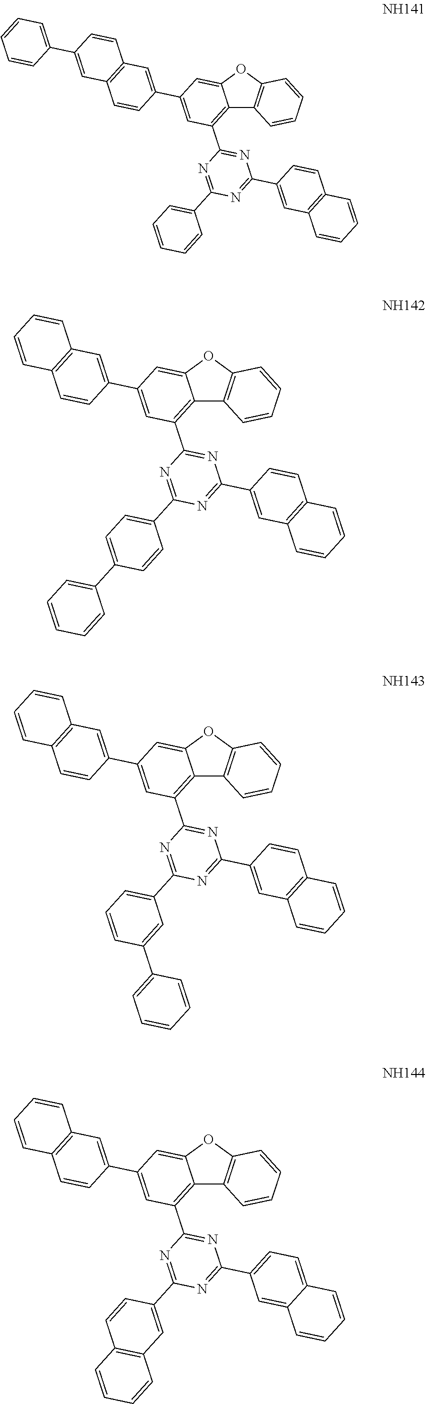 [SMILES: c1ccc(-c2ccc3cc(-c4cc(-c5nc(-c6ccccc6)nc(-c6ccc7ccccc7c6)n5)c5c(c4)oc4ccccc45)ccc3c2)cc1 ; c1ccc(-c2ccc(-c3nc(-c4ccc5ccccc5c4)nc(-c4cc(-c5ccc6ccccc6c5)cc5oc6ccccc6c45)n3)cc2)cc1 ; c1ccc(-c2cccc(-c3nc(-c4ccc5ccccc5c4)nc(-c4cc(-c5ccc6ccccc6c5)cc5oc6ccccc6c45)n3)c2)cc1 ; c1ccc2cc(-c3cc(-c4nc(-c5ccc6ccccc6c5)nc(-c5ccc6ccccc6c5)n4)c4c(c3)oc3ccccc34)ccc2c1]

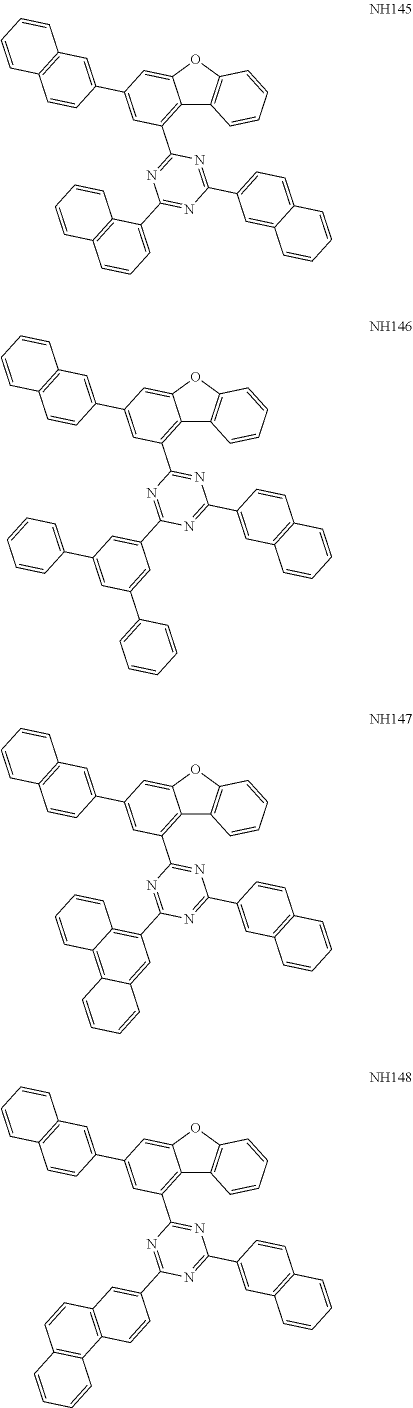 [SMILES: c1ccc2cc(-c3cc(-c4nc(-c5ccc6ccccc6c5)nc(-c5cccc6ccccc56)n4)c4c(c3)oc3ccccc34)ccc2c1 ; c1ccc(-c2cc(-c3ccccc3)cc(-c3nc(-c4ccc5ccccc5c4)nc(-c4cc(-c5ccc6ccccc6c5)cc5oc6ccccc6c45)n3)c2)cc1 ; c1ccc2cc(-c3cc(-c4nc(-c5ccc6ccccc6c5)nc(-c5cc6ccccc6c6ccccc56)n4)c4c(c3)oc3ccccc34)ccc2c1 ; c1ccc2cc(-c3cc(-c4nc(-c5ccc6ccccc6c5)nc(-c5ccc6c(ccc7ccccc76)c5)n4)c4c(c3)oc3ccccc34)ccc2c1]

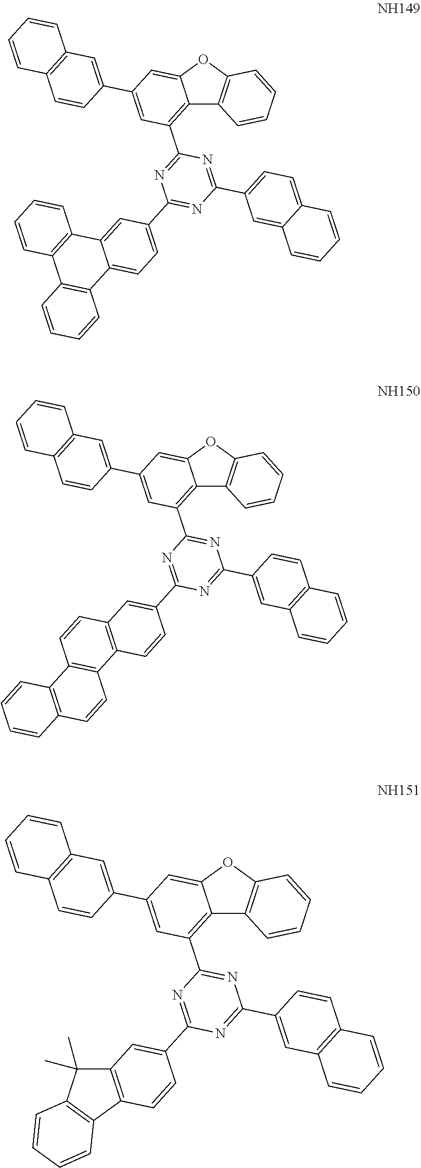 [SMILES: c1ccc2cc(-c3cc(-c4nc(-c5ccc6ccccc6c5)nc(-c5ccc6c7ccccc7c7ccccc7c6c5)n4)c4c(c3)oc3ccccc34)ccc2c1 ; c1ccc2cc(-c3cc(-c4nc(-c5ccc6ccccc6c5)nc(-c5ccc6c(ccc7c8ccccc8ccc67)c5)n4)c4c(c3)oc3ccccc34)ccc2c1 ; CC1(C)c2ccccc2-c2ccc(-c3nc(-c4ccc5ccccc5c4)nc(-c4cc(-c5ccc6ccccc6c5)cc5oc6ccccc6c45)n3)cc21]

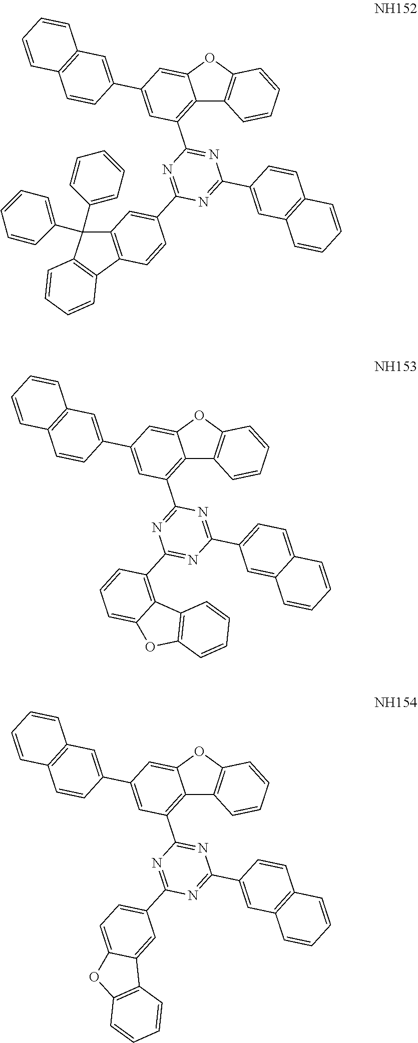 [SMILES: c1ccc(C2(c3ccccc3)c3ccccc3-c3ccc(-c4nc(-c5ccc6ccccc6c5)nc(-c5cc(-c6ccc7ccccc7c6)cc6oc7ccccc7c56)n4)cc32)cc1 ; c1ccc2cc(-c3cc(-c4nc(-c5ccc6ccccc6c5)nc(-c5cccc6oc7ccccc7c56)n4)c4c(c3)oc3ccccc34)ccc2c1 ; c1ccc2cc(-c3cc(-c4nc(-c5ccc6ccccc6c5)nc(-c5ccc6oc7ccccc7c6c5)n4)c4c(c3)oc3ccccc34)ccc2c1]

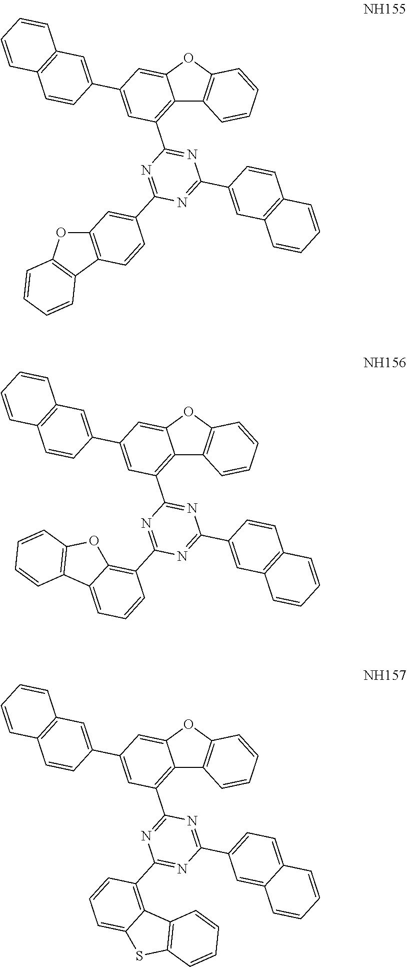 [SMILES: c1ccc2cc(-c3cc(-c4nc(-c5ccc6ccccc6c5)nc(-c5ccc6c(c5)oc5ccccc56)n4)c4c(c3)oc3ccccc34)ccc2c1 ; c1ccc2cc(-c3cc(-c4nc(-c5ccc6ccccc6c5)nc(-c5cccc6c5oc5ccccc56)n4)c4c(c3)oc3ccccc34)ccc2c1 ; c1ccc2cc(-c3cc(-c4nc(-c5ccc6ccccc6c5)nc(-c5cccc6sc7ccccc7c56)n4)c4c(c3)oc3ccccc34)ccc2c1]

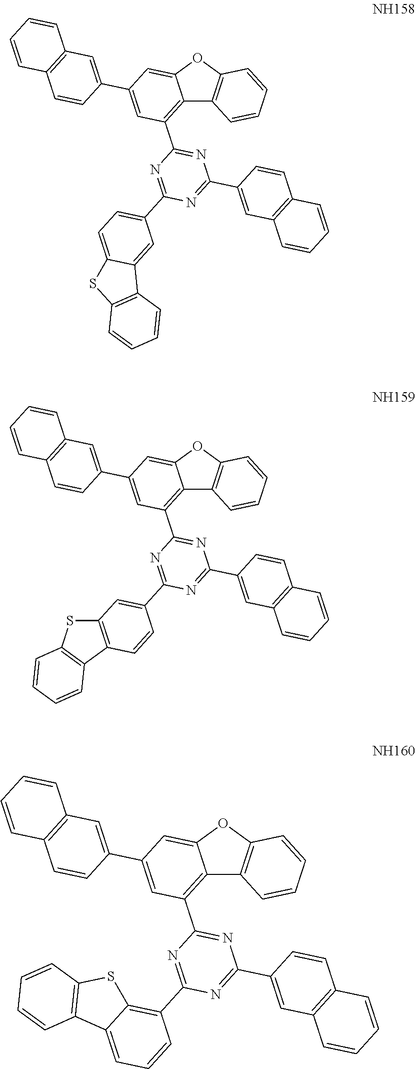 [SMILES: c1ccc2cc(-c3cc(-c4nc(-c5ccc6ccccc6c5)nc(-c5ccc6sc7ccccc7c6c5)n4)c4c(c3)oc3ccccc34)ccc2c1 ; c1ccc2cc(-c3cc(-c4nc(-c5ccc6ccccc6c5)nc(-c5ccc6c(c5)sc5ccccc56)n4)c4c(c3)oc3ccccc34)ccc2c1 ; c1ccc2cc(-c3cc(-c4nc(-c5ccc6ccccc6c5)nc(-c5cccc6c5sc5ccccc56)n4)c4c(c3)oc3ccccc34)ccc2c1]

The organic material layer of the organic light emitting device of the present invention may be composed of a single-layered structure, but may be composed of a multi-layered structure in which two or more organic material layers are stacked. For example, the organic light emitting device of the present invention may have a structure including a hole injection layer, a hole transport layer, a light emitting layer, an electron transport layer, an electron injection layer, and the like as organic material layers. However, the structure of the organic light emitting device is not limited thereto, and may include a fewer number of organic material layers.

In an exemplary embodiment of the present specification, the first electrode may be a positive electrode, and the second electrode may be a negative electrode.

In another exemplary embodiment of the present specification, the first electrode may be a negative electrode, and the second electrode may be a positive electrode.

The organic light emitting device according to an exemplary embodiment of the present specification may be manufactured by typical methods and materials for manufacturing an organic light emitting device, except that an organic material layer having one or more layers is formed by using the heterocyclic compound of the above-described Chemical Formula 1.

The heterocyclic compound of Chemical Formula 1 may be formed as an organic material layer by not only a vacuum deposition method, but also a solution application method when an organic light emitting device is manufactured. Herein, the solution coating method means spin coating, dip coating, inkjet printing, screen printing, a spray method, roll coating, and the like, but is not limited thereto.

In an exemplary embodiment of the present specification, the organic light emitting device may be a blue organic light emitting device, and the heterocyclic compound of Chemical Formula 1 may be used as a material for the blue organic light emitting device. For example, the heterocyclic compound of Chemical Formula 1 may be included in a hole transport layer, an electron blocking layer or a light emitting layer of the blue organic light emitting device.

In another exemplary embodiment of the present specification, the organic light emitting device may be a green organic light emitting device, and the heterocyclic compound of Chemical Formula 1 may be used as a material for the green organic light emitting device. For example, the heterocyclic compound of Chemical Formula 1 may be included in a hole transport layer, an electron blocking layer or a light emitting layer of the green organic light emitting device.

In still another exemplary embodiment of the present specification, the organic light emitting device may be a red organic light emitting device, and the heterocyclic compound of Chemical Formula 1 may be used as a material for the red organic light emitting device. For example, the heterocyclic compound of Chemical Formula 1 may be included in a hole transport layer, an electron blocking layer or a light emitting layer of the red organic light emitting device.

The organic light emitting device of the present invention may further include one or more layers selected from the group consisting of a light emitting layer, a hole injection layer, a hole transport layer, an electron injection layer, an electron transport layer, an electron blocking layer, and a hole blocking layer.

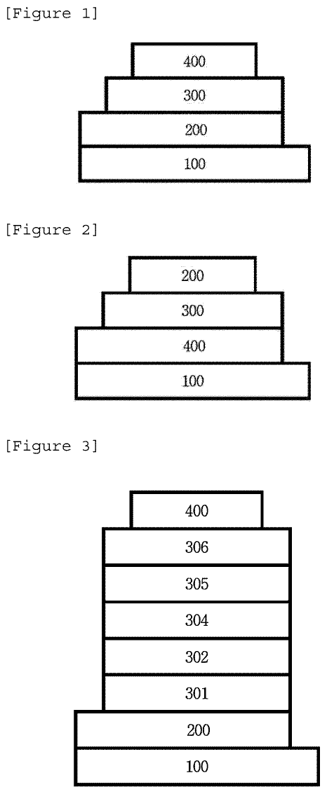

FIGS. 1 to 4 exemplify the stacking sequence of the electrodes and the organic material layer of the organic light emitting device according to an exemplary embodiment of the present specification. However, the scope of the present application is not intended to be limited by these drawings, and the structure of the organic light emitting device known in the art may also be applied to the present application.

According to FIG. 1, an organic light emitting device in which a positive electrode 200, an organic material layer 300, and a negative electrode 400 are sequentially stacked on a substrate 100 is illustrated. However, the organic light emitting device is not limited only to such a structure, and as in FIG. 2, an organic light emitting device in which a negative electrode, an organic material layer, and a positive electrode are sequentially stacked on a substrate may also be implemented.

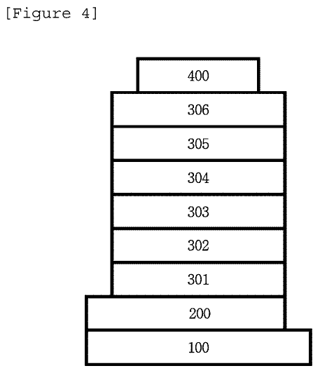

FIGS. 3 and 4 exemplify a case where an organic material layer is a multilayer. The organic light emitting device according to FIG. 3 includes a hole injection layer 301, a hole transport layer 302, a light emitting layer 304, an electron transport layer 305 and an electron injection layer 306, and the organic light emitting device according to FIG. 4 includes a hole injection layer 301, a hole transport layer 302, an electron blocking layer 303, a light emitting layer 304, an electron transport layer 305 and an electron injection layer 306. However, the scope of the present application is not limited by the stacking structure as described above, and if necessary, the other layers except for the light emitting layer may be omitted, and another necessary functional layer may be further added.

An organic material layer including the heterocyclic compound of Chemical Formula 1 may additionally include other materials, if necessary.

In the organic light emitting device according to an exemplary embodiment of the present specification, materials other than the heterocyclic compound of Chemical Formula 1 will be exemplified below, but these materials are illustrative only and are not for limiting the scope of the present application, and may be replaced with materials publicly known in the art.

As a positive electrode material, materials having a relatively high work function may be used, and a transparent conductive oxide, a metal or a conductive polymer, and the like may be used. Specific examples of the positive electrode material include: a metal such as vanadium, chromium, copper, zinc, and gold, or an alloy thereof; a metal oxide such as zinc oxide, indium oxide, indium tin oxide (ITO), and indium zinc oxide (IZO); a combination of a metal and an oxide, such as ZnO:Al or SnO₂:Sb; a conductive polymer such as poly(3-methylthiophene), poly[3,4-(ethylene-1,2-dioxy)thiophene](PEDOT), polypyrrole, and polyaniline; and the like, but are not limited thereto.

As a negative electrode material, materials having a relatively low work function may be used, and a metal, a metal oxide, or a conductive polymer, and the like may be used. Specific examples of the negative electrode material include: a metal such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto.

As a hole injection material, a publicly-known hole injection material may also be used, and it is possible to use, for example, a phthalocyanine compound such as copper phthalocyanine disclosed in U.S. Pat. No. 4,356,429 or starburst-type amine derivatives described in the document [Advanced Material, 6, p. 677 (1994)], for example, tris(4-carbazoyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl]benzene (m-MTDAPB), polyaniline/dodecylbenzenesulfonic acid or poly(3,4-ethylenedioxythiophene)/poly(4-styrenesulfonate), which is a soluble conductive polymer, polyaniline/camphor sulfonic acid or polyaniline/poly(4-styrenesulfonate), and the like.

As a hole transport material, a pyrazoline derivative, an arylamine-based derivative, a stilbene derivative, a triphenyldiamine derivative, and the like may be used, and a low-molecular weight or polymer material may also be used.

As an electron transport material, it is possible to use an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, a metal complex of 8-hydroxyquinoline and a derivative thereof, and the like, and a low-molecular weight material and a polymer material may also be used.

As an electron injection material, for example, LiF is representatively used in the art, but the present application is not limited thereto.

As a light emitting material, a red, green, or blue light emitting material may be used, and if necessary, two or more light emitting materials may be mixed and used. In this case, two or more light emitting materials are deposited and used as an individual supply source, or pre-mixed to be deposited and used as one supply source. Further, a fluorescent material may also be used as the light emitting material, but may also be used as a phosphorescent material. As the light emitting material, it is also possible to use alone a material which emits light by combining holes and electrons each injected from a positive electrode and a negative electrode, but materials in which a host material and a dopant material are involved in light emission together may also be used.

When hosts of the light emitting material are mixed and used, the same series of hosts may also be mixed and used, and different series of hosts may also be mixed and used. For example, any two or more materials from N-type host materials or P-type host materials may be selected and used as a host material for a light emitting layer.

The organic light emitting device according to an exemplary embodiment of the present specification may be a top emission type, a bottom emission type, or a dual emission type according to the material to be used.

The heterocyclic compound according to an exemplary embodiment of the present specification may act even in organic electronic devices including organic solar cells, organic photoconductors, organic transistors, and the like, based on the principle similar to those applied to organic light emitting devices.

In addition, it is possible to finely adjust an energy band-gap by introducing various substituents into the structure of Chemical Formula 1, and meanwhile, it is possible to improve characteristics at the interface between organic materials and diversify the use of the material.

In an exemplary embodiment of the present specification, provided is a composition for forming an organic material layer, including the heterocyclic compound.

In an exemplary embodiment of the present specification, the composition for forming an organic material layer may further include the compound of Chemical Formula 2.

In an exemplary embodiment of the present specification, the composition for forming an organic material layer may include the heterocyclic compound and the compound of Chemical Formula 2 at a weight ratio of 1:10 to 10:1.

In an exemplary embodiment of the present specification, provided is a method for manufacturing an organic light emitting device, the method including: preparing a substrate; forming a first electrode on the substrate; forming an organic material layer having one or more layers on the first electrode; and forming a second electrode on the organic material layer, in which the forming of the organic material layer includes forming the organic material layer having one or more layers by using the composition for forming an organic material layer, including the heterocyclic compound.

In an exemplary embodiment of the present specification, provided is a method for manufacturing an organic light emitting device, the method including: preparing a substrate; forming a first electrode on the substrate; forming an organic material layer having one or more layers on the first electrode; and forming a second electrode on the organic material layer, in which the forming of the organic material layer includes forming the organic material layer having one or more layers by using the composition for forming an organic material layer, including the heterocyclic compound and the compound of Chemical Formula 2 or 3.

In an exemplary embodiment of the present specification, provided is a method for manufacturing an organic light emitting device, in which the forming of the organic material layer forms the organic material layer by pre-mixing the heterocyclic compound represented by Chemical Formula 1 and the compound represented by Chemical Formula 2, and using a thermal vacuum deposition method.

The pre-mixing means that before the heterocyclic compound represented by Chemical Formula 1 and the compound represented by Chemical Formula 2 are deposited onto an organic material layer, the materials are first mixed and the mixture is contained in one common container and mixed.

The pre-mixed material may be referred to as a composition for forming an organic material layer according to an exemplary embodiment of the present specification.

### MODE FOR INVENTION

Hereinafter, the present specification will be described in more detail through Examples, but these Examples are provided only for exemplifying the present application, and are not intended to limit the scope of the present application.

### Preparation Examples

### <Preparation Example 1> Preparation of Compounds 4 and 65

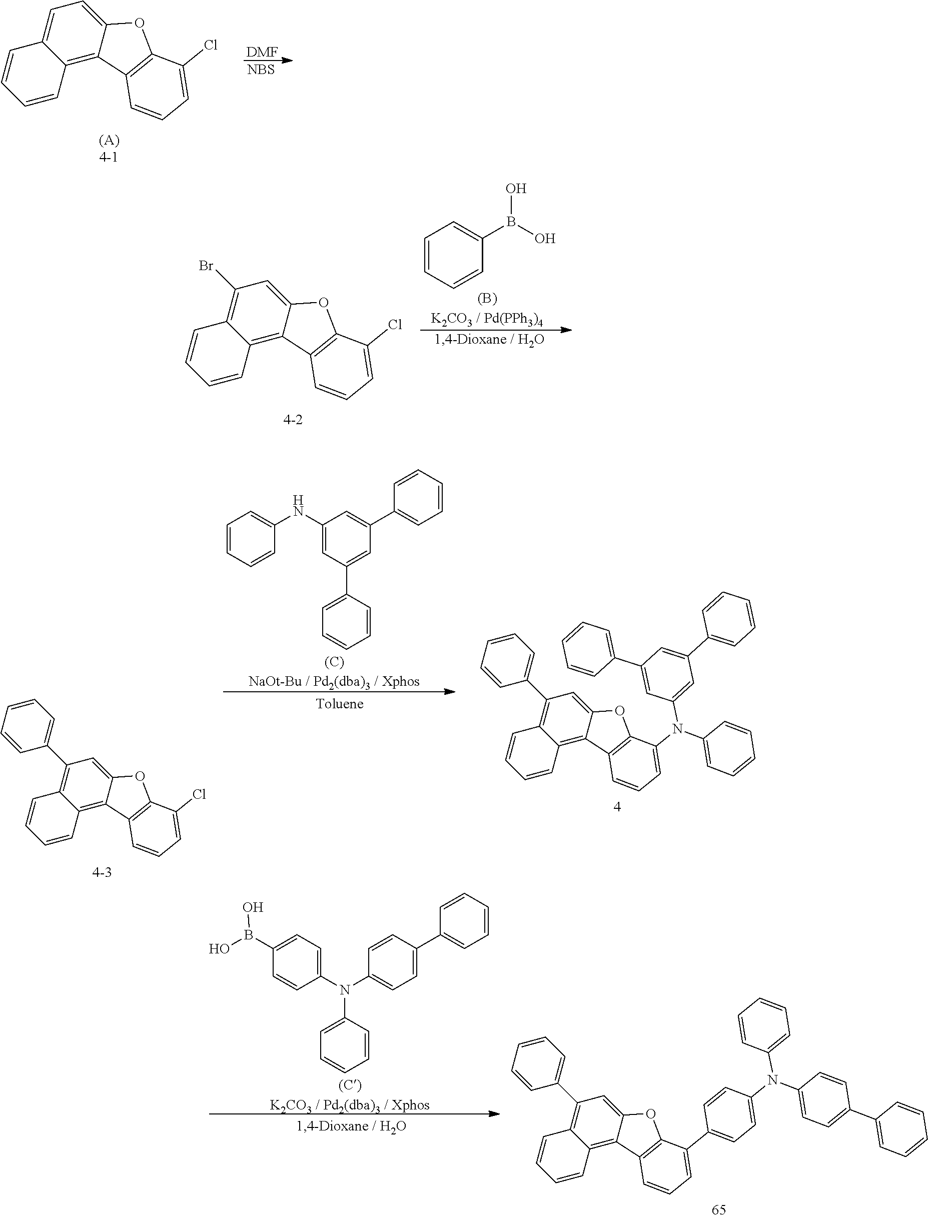

### 1) Preparation of Compound 4-2

Dimethylformamide (DMF) (200 ml) was put into 8-chloronaphtho[2,1-b]benzofuran (A) (15 g, 0.059 mol, 1 eq) and N-bromosuccinimide (NBS) (12.7 g, 0.07 1 mol, 1.2 eq), and the resulting mixture was stirred at 60° C. for 12 hours. After the reaction was terminated by adding water thereto, extraction was performed using methylene chloride (MC) and water. Thereafter, moisture was removed with MgSO₄. The residue was separated by silica gel column to obtain 13 g of Compound 4-2 with a yield of 66%.

### 2) Preparation of Compound 4-3

1,4-Dioxane (150 ml) and water (30 ml) were put into Compound 4-2 (13 g, 0.039 mol, 1 eq), phenylboronic acid (B) (5.3 g, 0.043 mol, 1.1 eq), K₂CO₃ (13.5 g, 0.098 mol, 2.5 eq), and Pd(PPh₃)₄ (tetrakis(triphenylphosphine)palladium(0)) (2.2 g, 0.002 mol, 0.05 eq), and the resulting mixture was stirred at 100° C. for 6 hours. After the reaction was terminated by adding water, extraction was performed using MC and water. Thereafter, moisture was removed with MgSO₄. The residue was separated by silica gel column to obtain 10 g of Compound 4-3 with a yield of 78%.

### 3) Preparation of Compound 4

Toluene (100 ml) was put into Compound 4-3 (5 g, 0.015 mol, 1 eq), N-phenyl-[1,1′: 3′,1″-terphenyl]-5′-amine (C) (5.4 g, 0.017 mol, 1.1 eq), sodium tert-butoxide (NaOt-Bu) (2.2 g, 0.023 mol, 1.5 eq), Pd₂(dba)₃ (tris(dibenzylideneacetone)dipalladium (0)) (0.7 g, 0.0008 mol, 0.05 eq), and Xphos (2-dicyclohexylphosphino-2′,4′,6′-triisopropylbiphenyl) (0.7 g, 0.0015 mol, 0.1 eq), and the resulting mixture was stirred at 100° C. for 8 hours. After the reaction was terminated by adding water, extraction was performed using MC and water. Thereafter, moisture was removed with MgSO₄. The residue was separated by silica gel column to obtain 8 g of Compound 4 with a yield of 86%.

### 4) Preparation of Compound 65

1,4-Dioxane (80 ml) and water (20 ml) were put into Compound 4-3 (5 g, 0.015 mol, 1 eq), (4-([1,1′-biphenyl]-4-yl(phenyl)amino)phenyl)boronic acid (C′) (5.8 g, 0.016 mol, 1.05 eq), K₂CO₃ (5.3 g, 0.038 mol, 2.5 eq), Pd₂(dba)₃ (0.7 g, 0.008 mol, 0.05 eq), and Xphos (0.7 g, 0.0015 mol, 0.1 eq), and the resulting mixture was stirred at 100° C. for 6 hours. After the reaction was terminated by adding water, extraction was performed using MC and water. Thereafter, moisture was removed with MgSO₄. The residue was separated by silica gel column to obtain 7 g of Compound 65 with a yield of 75%.

Compounds were synthesized in the same manner as in Preparation Example 1 using Intermediates A, B and C of the following Table 1 instead of (A), (B), (C) and (C′) in Preparation Example 1.

TABLE 1

| Com- | | | |
|---|---|---|---|
| pound | | | |
| No. | Intermediate A | Intermediate B | Intermediate C |
| 25 | 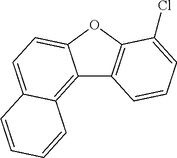 [SMILES: Clc1cccc2c1oc1ccc3ccccc3c12] | 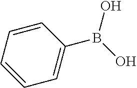 | 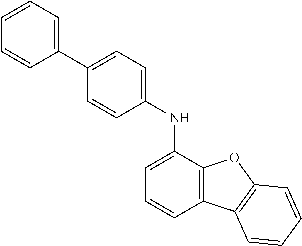 [SMILES: c1ccc(-c2ccc(Nc3cccc4c3oc3ccccc34)cc2)cc1] |
| 41 | 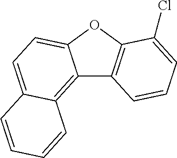 [SMILES: Clc1cccc2c1oc1ccc3ccccc3c12] | 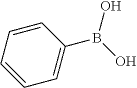 | 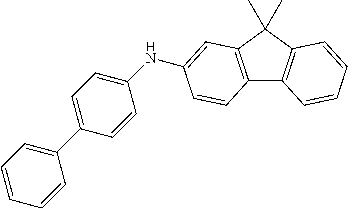 [SMILES: CC1(C)c2ccccc2-c2ccc(Nc3ccc(-c4ccccc4)cc3)cc21] |
| 93 | 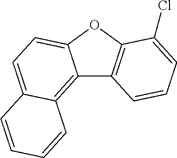 [SMILES: Clc1cccc2c1oc1ccc3ccccc3c12] | 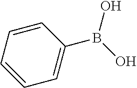 | 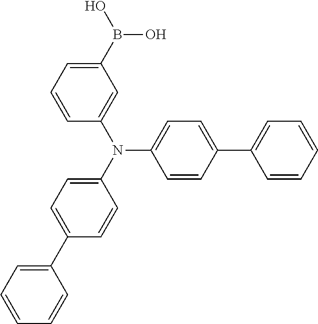 [SMILES: OB(O)c1cccc(N(c2ccc(-c3ccccc3)cc2)c2ccc(-c3ccccc3)cc2)c1] |
| 111 | 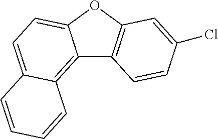 [SMILES: Clc1ccc2c(c1)oc1ccc3ccccc3c12] | 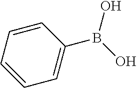 | 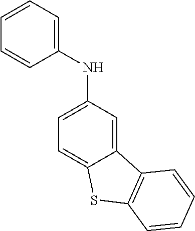 [SMILES: c1ccc(Nc2ccc3sc4ccccc4c3c2)cc1] |
| 135 | 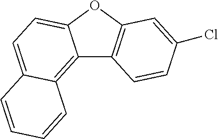 [SMILES: Clc1ccc2c(c1)oc1ccc3ccccc3c12] | 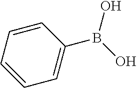 | 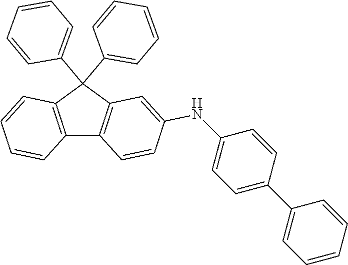 [SMILES: c1ccc(-c2ccc(Nc3ccc4c(c3)C(c3ccccc3)(c3ccccc3)c3ccccc3-4)cc2)cc1] |
| 157 | 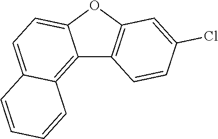 [SMILES: Clc1ccc2c(c1)oc1ccc3ccccc3c12] | 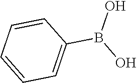 | 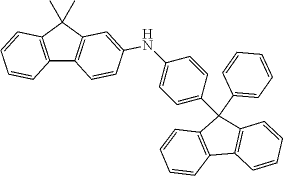 [SMILES: CC1(C)c2ccccc2-c2ccc(Nc3ccc(C4(c5ccccc5)c5ccccc5-c5ccccc54)cc3)cc21] |
| 178 | 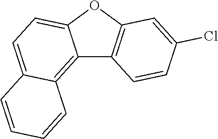 [SMILES: Clc1ccc2c(c1)oc1ccc3ccccc3c12] | 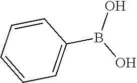 | 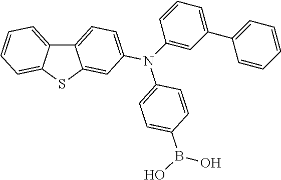 [SMILES: OB(O)c1ccc(N(c2cccc(-c3ccccc3)c2)c2ccc3c(c2)sc2ccccc23)cc1] |
| 185 | 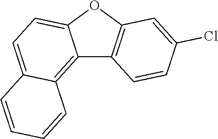 [SMILES: Clc1ccc2c(c1)oc1ccc3ccccc3c12] | 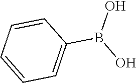 | 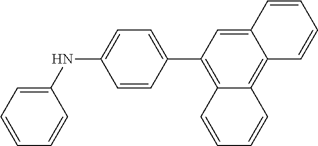 [SMILES: c1ccc(Nc2ccc(-c3cc4ccccc4c4ccccc34)cc2)cc1] |
| 207 | 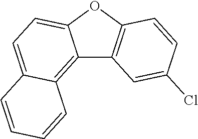 [SMILES: Clc1ccc2oc3ccc4ccccc4c3c2c1] | 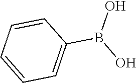 | 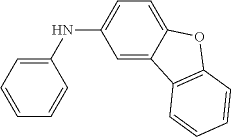 [SMILES: c1ccc(Nc2ccc3oc4ccccc4c3c2)cc1] |
| 233 | 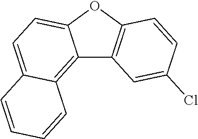 [SMILES: Clc1ccc2oc3ccc4ccccc4c3c2c1] | 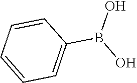 | 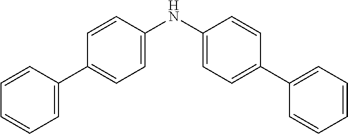 [SMILES: c1ccc(-c2ccc(Nc3ccc(-c4ccccc4)cc3)cc2)cc1] |
| 250 | 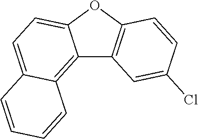 [SMILES: Clc1ccc2oc3ccc4ccccc4c3c2c1] | 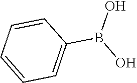 | 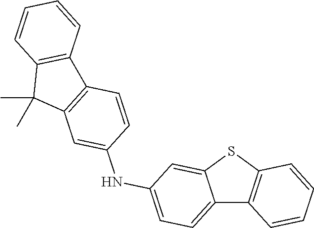 [SMILES: CC1(C)c2ccccc2-c2ccc(Nc3ccc4c(c3)sc3ccccc34)cc21] |
| 267 | 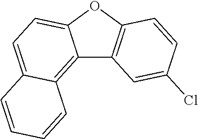 [SMILES: Clc1ccc2oc3ccc4ccccc4c3c2c1] | 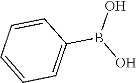 | 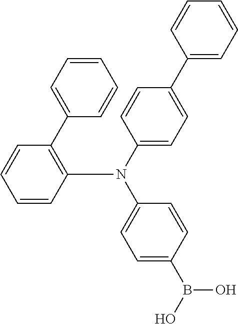 [SMILES: OB(O)c1ccc(N(c2ccc(-c3ccccc3)cc2)c2ccccc2-c2ccccc2)cc1] |
| 291 | 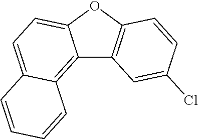 [SMILES: Clc1ccc2oc3ccc4ccccc4c3c2c1] | 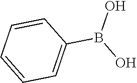 | 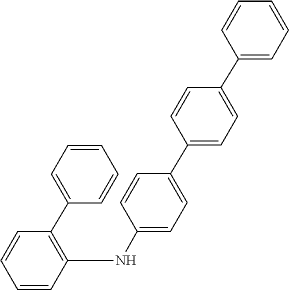 [SMILES: c1ccc(-c2ccc(-c3ccc(Nc4ccccc4-c4ccccc4)cc3)cc2)cc1] |
| 320 | 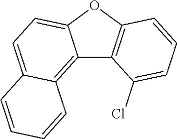 [SMILES: Clc1cccc2oc3ccc4ccccc4c3c12] | 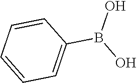 | 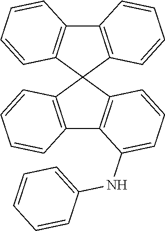 [SMILES: c1ccc(Nc2cccc3c2-c2ccccc2C32c3ccccc3-c3ccccc32)cc1] |
| 338 | 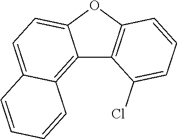 [SMILES: Clc1cccc2oc3ccc4ccccc4c3c12] | 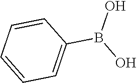 | 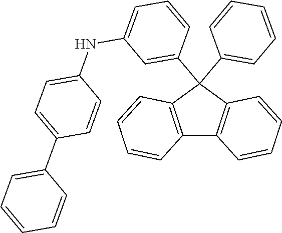 [SMILES: c1ccc(-c2ccc(Nc3cccc(C4(c5ccccc5)c5ccccc5-c5ccccc54)c3)cc2)cc1] |
| 341 | 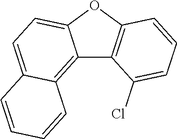 [SMILES: Clc1cccc2oc3ccc4ccccc4c3c12] | 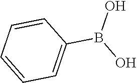 | 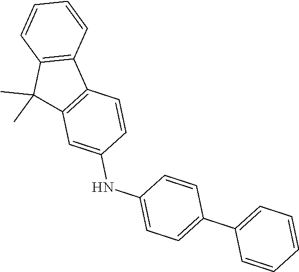 [SMILES: CC1(C)c2ccccc2-c2ccc(Nc3ccc(-c4ccccc4)cc3)cc21] |
| 368 | 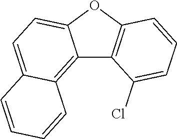 [SMILES: Clc1cccc2oc3ccc4ccccc4c3c12] | 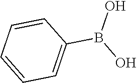 | 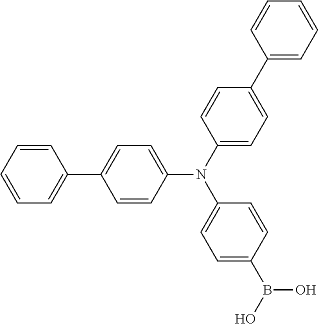 [SMILES: OB(O)c1ccc(N(c2ccc(-c3ccccc3)cc2)c2ccc(-c3ccccc3)cc2)cc1] |
| 397 | 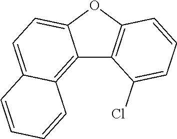 [SMILES: Clc1cccc2oc3ccc4ccccc4c3c12] | 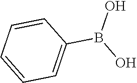 | 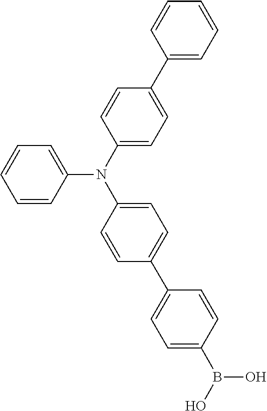 [SMILES: OB(O)c1ccc(-c2ccc(N(c3ccccc3)c3ccc(-c4ccccc4)cc3)cc2)cc1] |
| 402 | 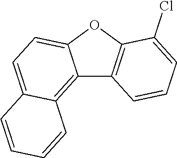 [SMILES: Clc1cccc2c1oc1ccc3ccccc3c12] | 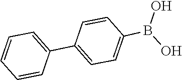 [SMILES: OB(O)c1ccc(-c2ccccc2)cc1] | 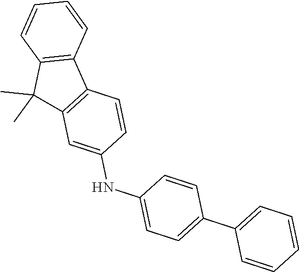 [SMILES: CC1(C)c2ccccc2-c2ccc(Nc3ccc(-c4ccccc4)cc3)cc21] |
| 426 | 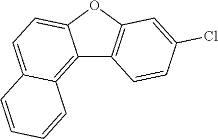 [SMILES: Clc1ccc2c(c1)oc1ccc3ccccc3c12] | 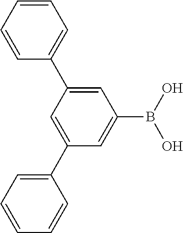 [SMILES: OB(O)c1cc(-c2ccccc2)cc(-c2ccccc2)c1] | 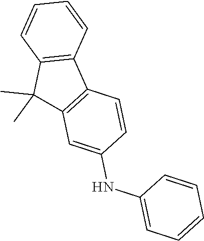 [SMILES: CC1(C)c2ccccc2-c2ccc(Nc3ccccc3)cc21] |
| 449 | 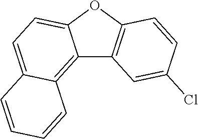 [SMILES: Clc1ccc2oc3ccc4ccccc4c3c2c1] | 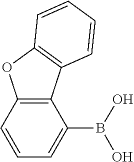 [SMILES: OB(O)c1cccc2oc3ccccc3c12] | 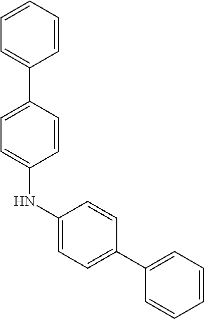 [SMILES: c1ccc(-c2ccc(Nc3ccc(-c4ccccc4)cc3)cc2)cc1] |
| 480 | 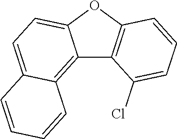 [SMILES: Clc1cccc2oc3ccc4ccccc4c3c12] | 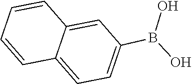 [SMILES: OB(O)c1ccc2ccccc2c1] | 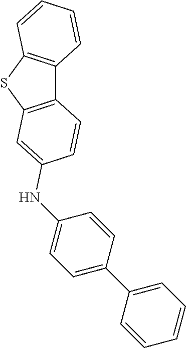 [SMILES: c1ccc(-c2ccc(Nc3ccc4c(c3)sc3ccccc34)cc2)cc1] |
| 481 | 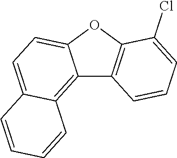 [SMILES: Clc1cccc2c1oc1ccc3ccccc3c12] | 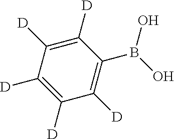 [SMILES: [2H]c1c([2H])c([2H])c(B(O)O)c([2H])c1[2H]] | 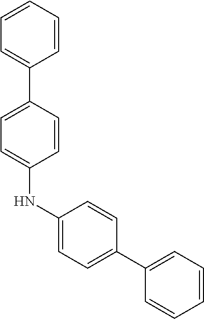 [SMILES: c1ccc(-c2ccc(Nc3ccc(-c4ccccc4)cc3)cc2)cc1] |
| 484 | 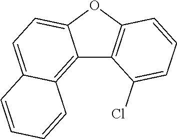 [SMILES: Clc1cccc2oc3ccc4ccccc4c3c12] | 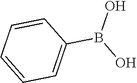 | 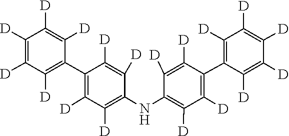 [SMILES: [2H]c1c([2H])c([2H])c(-c2c([2H])c([2H])c(Nc3c([2H])c([2H])c(-c4c([2H])c([2H])c([2H])c([2H])c4[2H])c([2H])c3[2H])c([2H])c2[2H])c([2H])c1[2H]] |
| 491 | 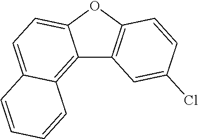 [SMILES: Clc1ccc2oc3ccc4ccccc4c3c2c1] | 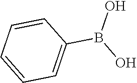 | 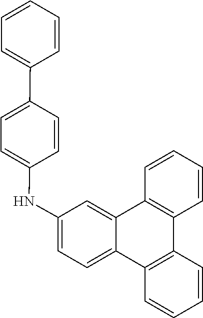 [SMILES: c1ccc(-c2ccc(Nc3ccc4c5ccccc5c5ccccc5c4c3)cc2)cc1] |

### <Preparation Example 2> Preparation of Compound 483

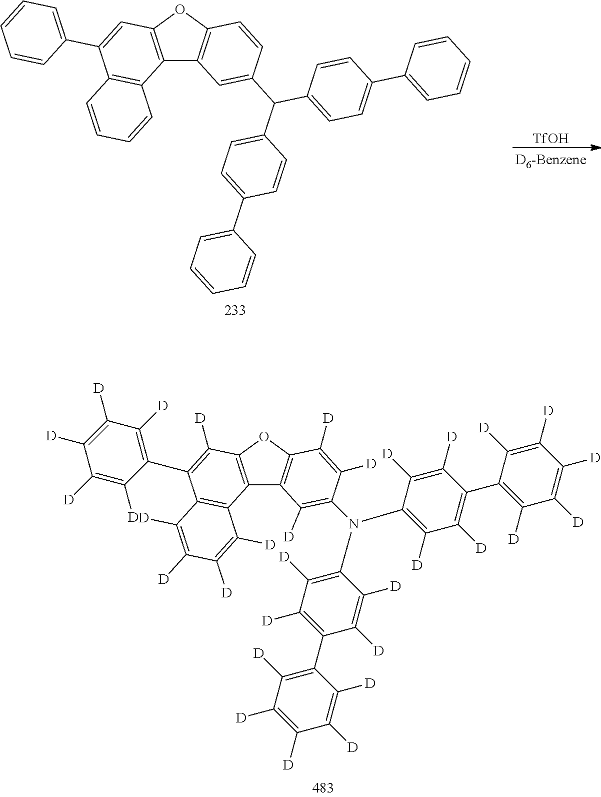

After Compound 233 (7 g, 0.011 mol, 1 eq) prepared in Preparation Example 1, triflic acid (TfOH) (2.6 g, 0.017 mol, 1.5 eq) and D₆-benzene (70 ml) were put into a flask, the resulting mixture was stirred at 100° C. for 8 hours. After the reaction was terminated by adding water, extraction was performed using MC and water. Thereafter, moisture was removed with MgSO₄. The residue was separated by silica gel column to obtain 6.2 g of Compound 483 with a yield of 84%.

### <Preparation Example 3> Preparation of Compound Nh9

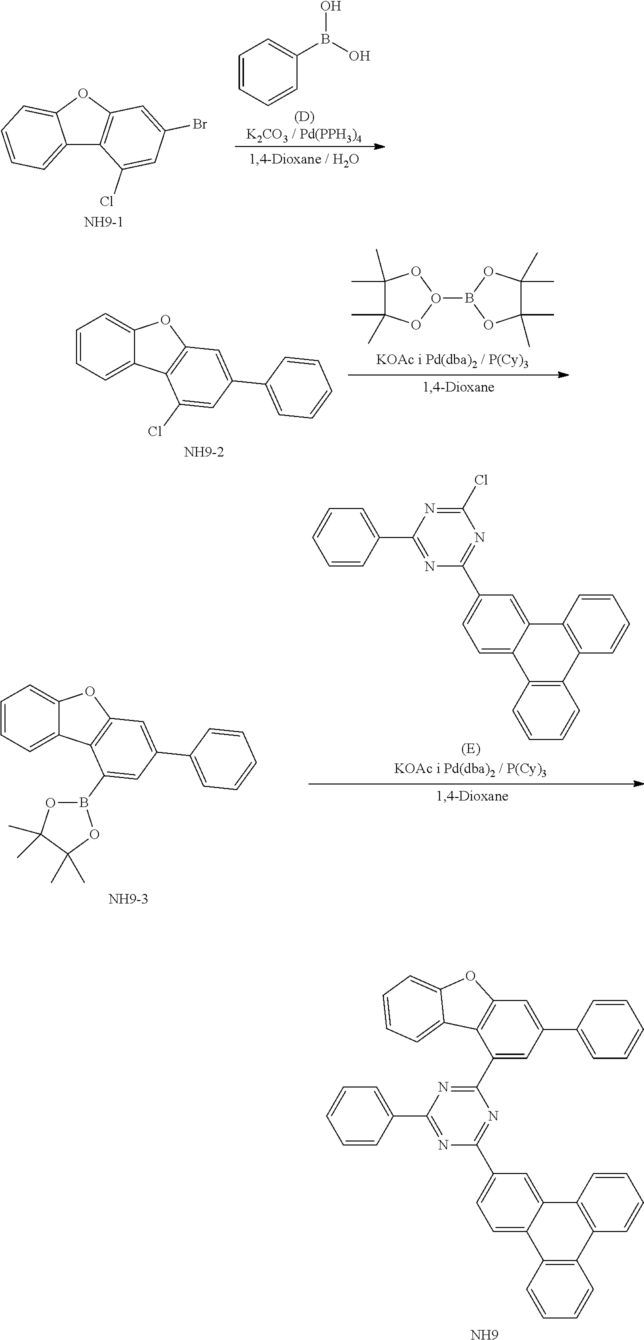

### 1) Preparation of Compound NH9-2

1,4-Dioxane (200 ml) and water (50 ml) were put into 3-bromo-1-chlorodibenzo[b,d]furan (20 g, 0.071 mol, 1 eq), phenylboronic acid (D) (9.5 g, 0.078 mol, 1.1 eq), K₂CO₃ (24.5 g, 0.178 mol, 2.5 eq), and Pd(PPh₃)₄ (4.1 g, 0.004 mol, 0.05 eq), and the resulting mixture was stirred at 100° C. for 7 hours. After the reaction was terminated by adding water, extraction was performed using MC and water. Thereafter, moisture was removed with MgSO₄. The residue was separated by silica gel column to obtain 15 g of Compound NH9-2 with a yield of 76%.

### 2) Preparation of Compound NH9-3

1,4-Dioxane (150 ml) was put into Compound NH9-2 (15 g, 0.054 mol, 1 eq), bis(pinacolato)diboron (20 g, 0.081 mol, 1.5 eq), KOAc (13.5 g, 0.098 mol, 2.5 eq), Pd(dba)₂ (1.5 g, 0.003 mol, 0.05 eq), and P(Cy)₃ (1.5 g, 0.0005 mol, 0.1 eq), and the resulting mixture was stirred at 100° C. for 8 hours. After the reaction was terminated by adding water, extraction was performed using MC and water. Thereafter, moisture was removed with MgSO₄. The residue was separated by silica gel column to obtain 12 g of Compound NH9-3 with a yield of 60%.

### 3) Preparation of Compound NH9

1,4-Dioxane (100 ml) and water (20 ml) were put into Compound NH9-3 (7.8 g, 0.021 mol, 1.1 eq), 2-chloro-4-phenyl-6-(triphenylen-2-yl)-1,3,5-triazine (E) (8 g, 0.019 mol, 1.1 eq), 1,4-dioxane(9.5 g, 0.078 mol, 1.1 eq), K₂CO₃ (6.6 g, 0.048 mol, 2.5 eq) and Pd(PPh₃)₄ (1.1 g, 0.001 mol, 0.05 eq), and the resulting mixture was stirred at 100° C. for 6 hours. After the reaction was terminated by adding water, extraction was performed using MC and water. Thereafter, moisture was removed with MgSO₄. The residue was separated by silica gel column to obtain Compound NH9 with a yield of 76%.

Compounds were synthesized in the same manner as in Preparation Example 3 using Intermediates D and E of the following Table 2 instead of Compounds (D) and (E) used in Preparation Example 3.

TABLE 2

| Com- | | |
|---|---|---|
| pound | | |
| No. | Intermediate D | Intermediate E |
| NH48 | 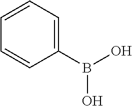 | 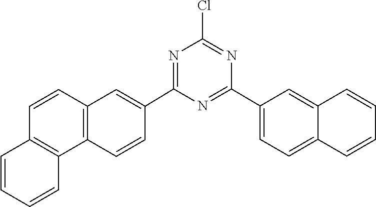 [SMILES: Clc1nc(-c2ccc3ccccc3c2)nc(-c2ccc3c(ccc4ccccc43)c2)n1] |
| NH57 | 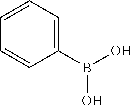 | 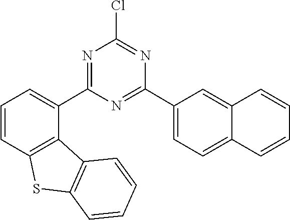 [SMILES: Clc1nc(-c2ccc3ccccc3c2)nc(-c2cccc3sc4ccccc4c23)n1] |
| NH84 | 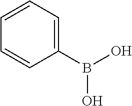 | 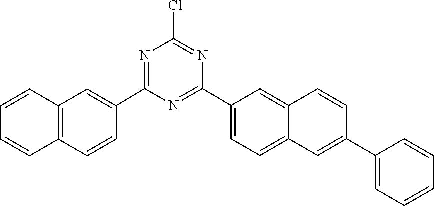 [SMILES: Clc1nc(-c2ccc3ccccc3c2)nc(-c2ccc3cc(-c4ccccc4)ccc3c2)n1] |
| NH115 | 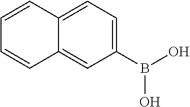 [SMILES: OB(O)c1ccc2ccccc2c1] | 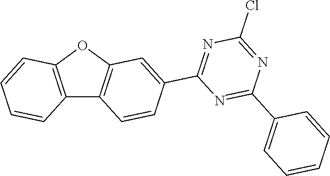 [SMILES: Clc1nc(-c2ccccc2)nc(-c2ccc3c(c2)oc2ccccc23)n1] |
| NH121 | 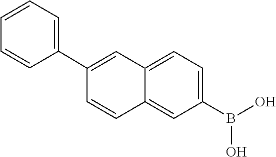 [SMILES: OB(O)c1ccc2cc(-c3ccccc3)ccc2c1] | 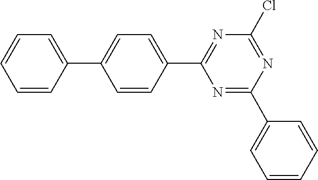 [SMILES: Clc1nc(-c2ccccc2)nc(-c2ccc(-c3ccccc3)cc2)n1] |
| NH131 | 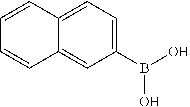 [SMILES: OB(O)c1ccc2ccccc2c1] | 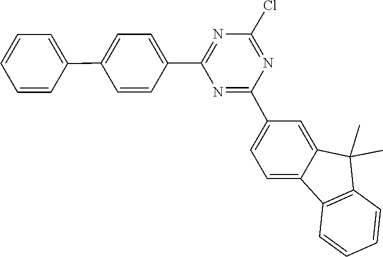 [SMILES: CC1(C)c2ccccc2-c2ccc(-c3nc(Cl)nc(-c4ccc(-c5ccccc5)cc4)n3)cc21] |
| NH141 | 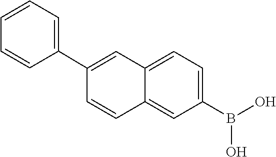 [SMILES: OB(O)c1ccc2cc(-c3ccccc3)ccc2c1] | 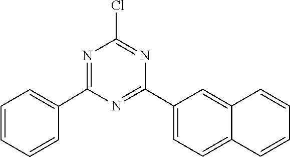 [SMILES: Clc1nc(-c2ccccc2)nc(-c2ccc3ccccc3c2)n1] |

The other compounds other than the compounds described in Preparation Examples 1 to 3 were also prepared in the same manner as in the above-described Preparation Examples, and synthesis results are shown in the following Tables 3 and 4. The following Table 3 shows the measured values of ¹H NMR (CDCl₃, 200 Mz), and the following Table 4 shows the measured values of field desorption mass spectrometry (FD-MS).

TABLE 3

| Compound | ¹H NMR (CDCl₃, 200 Mz) |
|---|---|
| 4 | δ = 8.55 (2H, m), 7.79 (2H, m), 7.64 (1H, s), |
| | 7.55~7.41 (15H, m), 7.20 (3H, m), 7.06 (2H, m), |
| | 6.81 (3H, m), 6.63 (2H, m), 6.39 (1H, d) |
| 25 | δ = 8.55 (2H, m), 7.89 (1H, d), 7.79 (2H, m), 7.64 (2H, |
| | m), 7.55~7.25 (16H, m), 7.07 (2H, m), 6.69 (2H, m), |
| | 6.63 (2H, m) |
| 41 | δ = 8.55 (2H, m), 7.87 (1H, d), 7.79 (2H, m), |
| | 7.64~7.38 (16H, m), 7.28 (2H, m), 7.07 (1H, t), |
| | 6.69 (3H, m), 6.58 (1H, d), 6.39 (1H, d), 1.72 (6H, s) |
| 65 | δ = 8.55 (2H, m), 7.85 (4H, m), 7.64 (1H, s), |
| | 7.55~7.38 (15H, m), 7.20 (2H, m), 6.81 (1H, t), |
| | 6.63 (6H, m) |
| 93 | δ = 8.55 (2H, m), 7.81 (4H, m), 7.64 (1H, s), |
| | 7.55~7.38 (21H, m), 6.88 (2H, m), 6.69 (4H, m), |
| | 6.59 (1H, d) |
| 111 | δ = 8.55 (2H, m), 8.45 (1H, d), 7.98 (1H, d), 7.73 (3H, |
| | m), 7.64~7.40 (11H, m), 7.20 (2H, m), 6.86 (2H, m), |
| | 6.63 (2H, m), 6.33 (1H, d) |
| 135 | δ = 8.55 (2H, m), 7.87 (1H, d), 7.79 (2H, m), |
| | 7.64~7.26 (25H, m), 7.11 (4H, m), 6.75 (3H, m), |
| | 6.58 (1H, d), 6.33 (1H, d) |
| 157 | δ = 8.55 (2H, m), 7.87 (5H, m), 7.64 (10H, m), 7.38 (11H, |
| | m), 7.11 (2H, m), 6.75 (3H, m), 6.58 (3H, m), 6.33 (1H, |
| | d), 1.72 (6H, s) |
| 178 | δ = 8.55 (2H, m), 8.45 (1H, d), 7.98 (2H, m), 7.79 (4H, |
| | m), 7.54 (17H, m), 7.06 (1H, s), 6.88 (3H, m), 6.69 (2H, |
| | m), 6.59 (1H, d) |
| 185 | δ = 8.93 (2H, m), 8.55 (2H, m), 8.12 (2H, m), 7.88 (7H, |
| | m), 7.54 (10H, m), 7.20 (2H, m), 6.81 (1H, t), 7.63 (4H, |
| | m), 6.33 (1H, d) |
| 207 | δ = 8.55 (2H, m), 7.89 (1H, d), 7.79 (2H, m), 7.65 (4H, |
| | m), 7.51 (9H, m), 7.20 (2H, m), 6.81 (1H, t), 7.63 (2H, |
| | m), 6.39 (2H, m) |
| 233 | δ = 8.55 (2H, m), 7.79 (2H, m), 7.65 (2H, m), 7.55 (20H, |
| | m), 6.69 (4H, m), 6.39 (1H, d) |
| 250 | δ = 8.55 (2H, m), 8.45 (1H, d), 7.98 (1H, d), 7.87 (4H, |
| | m), 7.65~7.38 (13H, m), 7.28 (1H, t), 7.06 (1H, s), |
| | 6.88 (1H, d), 6.75 (1H, s), 6.58 (1H, d), 6.39 (1H, d), |
| | 1.72 (6H, s) |
| 267 | δ = 8.55 (2H, m), 7.72 (5H, m), 7.64 (1H, s), 7.54 (18H, |
| | m), 7.16 (3H, m), 6.87 (1H, t), 6.69 (5H, m) |
| 291 | δ = 8.55 (2H, m), 7.79 (2H, m), 7.65 (2H, m), 7.51 (17H, |
| | m), 7.25 (4H, m), 7.08 (3H, m), 6.87 (1H, t), 6.69 (3H, |
| | m), 6.39 (1H, d) |
| 320 | δ = 8.55 (2H, m), 7.75 (5H, m), 7.64 (1H, s), 7.55 (5H, |
| | m), 7.41~7.16 (12H, m), 7.02 (2H, m), 6.91 (1H, d), |
| | 6.81 (1H, t), 6.63 (3H, m), 6.33 (1H, d) |
| 338 | δ = 8.55 (2H, m), 7.87 (2H, m), 7.79 (2H, m), 7.64 (1H, |
| | s), 7.54~7.26 (21H, m), 7.02 (5H, m), 6.69 (2H, m), |
| | 6.55 (1H, s), 6.44 (2H, m), 6.33 (1H, d) |
| 341 | δ = 8.55 (2H, m), 7.87 (1H, d), 7.79 (2H, m), |
| | 7.64~7.38 (16H, m), 7.28 (1H, t), 7.13 (1H, t), |
| | 7.02 (1H, d), 6.89 (3H, m) 6.58 (1H, d), 6.33 (1H, d), |
| | 1.72 (6H, s) |
| 368 | δ = 8.55 (2H, m), 7.75 (3H, m), 7.62~7.44 (24H, m), |
| | 6.69 (6H, m) |
| 397 | δ = 8.55 (2H, m), 7.75 (3H, m), 7.62~7.44 (17H, m), |
| | 7.20 (6H, m), 6.81 (1H, t), 6.69 (6H, m) |
| 402 | δ = 8.55 (2H, m), 7.87 (1H, d), 7.64~7.38 (18H, m), |
| | 7.25 (6H, m), 7.07 (1H, t), 6.69 (3H, m), 6.58 (1H, d), |
| | 6.39 (1H, d), 1.72 (6H, s) |
| 426 | δ = 8.55 (2H, m), 7.87 (1H, d), 7.66~7.38 (21H, m), |
| | 7.28 (3H, m), 6.81 (2H, m), 6.63 (3H, m), 6.33 (1H, d), |
| | 1.72 (6H, s) |
| 449 | δ = 8.55 (2H, m), 7.89 (1H, d), 7.75 (1H, d), |
| | 7.66~7.41 (24H, m), 6.69 (4H, m), 6.39 (1H, d) |
| 480 | δ = 8.55 (2H, m), 8.45 (1H, d), 8.00 (4H, m), 7.80 (1H, |
| | d), 7.73 (1H, d), 7.64~7.41 (15H, m), 7.13 (1H, t), |
| | 7.02 (2H, m), 6.88 (1H, d), 6.69 (2H, m), 6.33 (1H, d) |
| 481 | δ = 8.55 (2H, m), 7.64 (1H, s), 7.55 (16H, m), 7.25 (1H, |
| | d), 7.07 (1H, t), 6.69 (4H, m), 6.39 (1H, d) |
| 483 | δ = deuterium content of 100% with no ¹H NMR peak |
| 484 | δ = 8.55 (2H, m), 7.79 (2H, m), 7.64 (1H, s), 7.51 (5H, |
| | m), 7.13 (1H, t), 7.02 (1H, d), 6.38 (1H, d) |
| 491 | δ = 8.93 (2H, m), 8.55 (2H, m), 8.12 (3H, m), 7.86 (7H, |
| | m), 7.64 (2H, m), 7.41 (13H, m), 7.02 (1H, d), 6.69 (2H, |
| | m), 6.39 (1H, d) |
| NH9 | δ = 9.15 (1H, s), 8.93 (2H, m), 8.28 (2H, m), 8.12 (3H, |
| | m), 8.04 (1H, d), 7.89 (5H, m), 7.66~7.32 (13H, m) |
| NH48 | δ = 9.09 (1H, s), 8.99 (2H, m), 8.49 (1H, d), 8.32 (1H, |
| | s), 8.10 (2H, m), 8.00 (6H, m), 7.71~7.32 (14H, m) |
| NH57 | δ = 9.09 (1H, s), 8.45 (2H, m), 7.82 (7H, m), |
| | 7.66~7.32 (15H, m) |
| NH84 | δ = 9.09 (2H, m), 8.49 (2H, m), 7.89 (6H, m), |
| | 7.73~7.32 (19H, m) |
| NH115 | δ = 8.28 (2H, m), 7.95 (6H, m), 7.73~7.51 (12H, m), |
| | 7.38 (5H, m), |
| NH121 | δ = 8.28 (2H, m), 7.85 (5H, m), 7.73 (3H, m), |
| | 7.60~7.25 (21H, m), |
| NH131 | δ = 7.93 (8H, m), 7.77 (14H, m), 7.41 (7H, m), 1.72 (6H, |
| | m) |
| NH141 | δ = 9.09 (1H, s), 8.49 (1H, d), 8.28 (2H, d), 7.92 (6H, |
| | m), 7.73~7.32 (19H, m) |

TABLE 4

| Compound | FD-MS |
|---|---|
| 4 | m/z = 613.24 |
| 25 | m/z = 627.22 |
| 41 | m/z = 653.27 |
| 65 | m/z = 613.24 |
| 93 | m/z = 689.27 |
| 111 | m/z = 567.17 |
| 135 | m/z = 777.30 |
| 157 | m/z = 817.33 |
| 178 | m/z = 719.23 |
| 185 | m/z = 637.24 |
| 207 | m/z = 551.19 |
| 233 | m/z = 613.24 |
| 250 | m/z = 683.23 |
| 267 | m/z = 689.27 |
| 291 | m/z = 689.27 |
| 320 | m/z = 699.26 |
| 338 | m/z = 777.30 |
| 341 | m/z = 653.27 |
| 368 | m/z = 689.27 |
| 397 | m/z = 689.27 |
| 402 | m/z = 729.30 |
| 426 | m/z = 729.30 |
| 449 | m/z = 703.25 |
| 480 | m/z = 693.21 |
| 481 | m/z = 618.27 |
| 483 | m/z = 644.44 |
| 484 | m/z = 631.35 |
| 491 | m/z = 687.26 |
| NH9 | m/z = 625.22 |
| NH48 | m/z = 625.22 |
| NH57 | m/z = 631.17 |
| NH84 | m/z = 651.23 |
| NH115 | m/z = 615.19 |
| NH121 | m/z = 677.25 |
| NH131 | m/z = 717.28 |
| NH141 | m/z = 651.23 |

### Experimental Example 1

### 1) Manufacture of Organic Light Emitting Device

### Comparative Example 1

Trichloroethylene, acetone, ethanol, and distilled water were each sequentially used to ultrasonically wash a transparent electrode indium tin oxide (ITO) thin film obtained from glass for OLED (manufactured by Samsung-Corning Co., Ltd.) for 5 minutes, and then the ITO thin film was placed in isopropanol, stored, and then used. Next, the ITO substrate was disposed in a substrate folder of a vacuum deposition apparatus, and the following 4,4′,4″-tris(N,N-(2-naphthyl)-phenylamino)triphenyl amine (2-TNATA) was placed in a cell in the vacuum deposition apparatus.

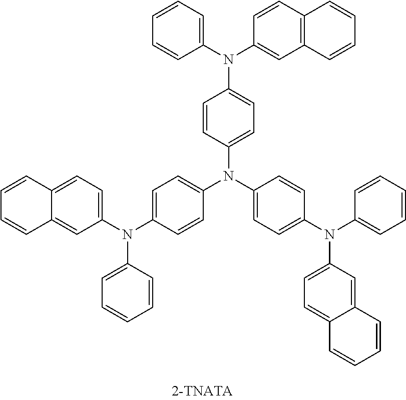 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, air in the chamber was evacuated until the degree of vacuum in the chamber reached 10⁻⁶ torr, and then a hole injection layer having a thickness of 600 Å was deposited on the ITO substrate by applying current to the cell to evaporate 2-TNATA. A hole transport layer having a thickness of 300 Å was deposited on the hole injection layer by placing the following N,N′-bis(α-naphthyl)—N,N′-diphenyl-4,4′-diamine (NPB) in another cell in the vacuum deposition apparatus and applying current to the cell to evaporate NPB.

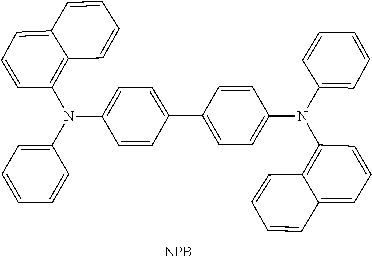 [SMILES: c1ccc(N(c2ccc(-c3ccc(N(c4ccccc4)c4cccc5ccccc45)cc3)cc2)c2cccc3ccccc23)cc1 ; "NPB"]

The hole injection layer and the hole transport layer were formed as described above, and then a blue light emitting material having the following structure as a light emitting layer was deposited thereon. Specifically, a blue light emitting host material BH1 was vacuum deposited to have a thickness of 200 Å on one cell in the vacuum deposition apparatus, and a blue light emitting dopant material D1 was vacuum deposited thereon in an amount of 5% based on the host material.

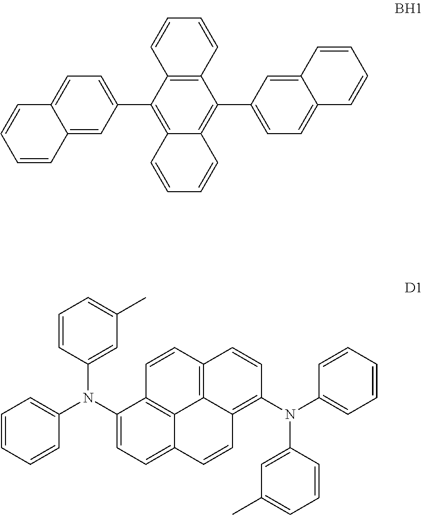

Subsequently, a compound having the following structural formula E1 as an electron transport layer was deposited to have a thickness of 300 Å.

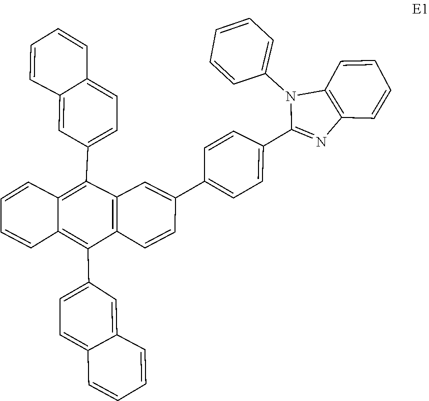 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

An OLED device was manufactured by depositing lithium fluoride (LiF) as an electron injection layer to have a thickness of 10 Å and allowing the Al negative electrode to have a thickness of 1,000 Å. Meanwhile, all the organic compounds required for manufacturing an OLED device were subjected to vacuum sublimed purification under 10⁻⁶ to 10⁻⁸ torr for each material, and used for the manufacture of OLED.

### Examples 1 to 28 and Comparative Examples 2 to 9

An organic electroluminescence device was manufactured in the same manner as in Experimental Example 1, except that the compound in the following Table 5 was used instead of NPB used when a hole transport layer was formed in Experimental Example 1.

### 2) Performance Evaluation of Organic Light Emitting Device

For the organic electroluminescence device manufactured as described above, electroluminescence (EL) characteristics were measured by M7000 manufactured by McScience Inc., and based on the measurement result thereof, T₉₅ was measured by a service life measurement device (M6000) manufactured by McScience Inc., when the reference luminance was 6,000 cd/m².

The results of measuring the driving voltage, light emitting efficiency, color coordinate (CIE) and service life of the blue organic light emitting device manufactured according to the present invention are shown in the following Table 5.

TABLE 5

| | | | Light | | |
|---|---|---|---|---|---|
| | | Driving | emitting | | Service |
| | | voltage | efficiency | CIE | life |
| | Compound | (V) | (cd/A) | (x, y) | (T₉₅) |
| Example 1 | 4 | 4.8 | 6.0 | (0.14, 0.05) | 73 |
| Example 2 | 25 | 4.6 | 6.2 | (0.14, 0.05) | 66 |
| Example 3 | 41 | 4.6 | 6.1 | (0.14, 0.05) | 78 |
| Example 4 | 65 | 4.7 | 6.1 | (0.14, 0.05) | 65 |
| Example 5 | 93 | 4.6 | 6.3 | (0.14, 0.05) | 72 |
| Example 6 | 111 | 4.8 | 6.2 | (0.14, 0.05) | 62 |
| Example 7 | 135 | 4.6 | 6.3 | (0.14, 0.05) | 65 |
| Example 8 | 157 | 4.7 | 6.3 | (0.14, 0.05) | 75 |
| Example 9 | 178 | 4.6 | 6.0 | (0.14, 0.05) | 73 |
| Example 10 | 185 | 4.8 | 6.1 | (0.14, 0.05) | 67 |
| Example 11 | 207 | 4.9 | 6.1 | (0.14, 0.05) | 74 |
| Example 12 | 233 | 4.8 | 6.2 | (0.14, 0.05) | 73 |
| Example 13 | 250 | 4.6 | 6.3 | (0.14, 0.05) | 63 |
| Example 14 | 267 | 4.7 | 6.3 | (0.14, 0.05) | 66 |
| Example 15 | 291 | 4.8 | 6.1 | (0.14, 0.05) | 76 |
| Example 16 | 320 | 4.9 | 6.1 | (0.14, 0.05) | 79 |
| Example 17 | 338 | 4.6 | 6.3 | (0.14, 0.05) | 77 |
| Example 18 | 341 | 4.7 | 6.2 | (0.14, 0.05) | 72 |
| Example 19 | 368 | 4.6 | 6.1 | (0.14, 0.05) | 67 |
| Example 20 | 397 | 4.8 | 6.0 | (0.14, 0.05) | 73 |
| Example 21 | 402 | 4.9 | 6.2 | (0.14, 0.05) | 71 |
| Example 22 | 426 | 4.6 | 6.0 | (0.14, 0.05) | 65 |
| Example 23 | 449 | 4.6 | 6.1 | (0.14, 0.05) | 63 |
| Example 24 | 480 | 4.7 | 6.0 | (0.14, 0.05) | 75 |
| Example 25 | 481 | 4.7 | 6.3 | (0.14, 0.05) | 79 |
| Example 26 | 483 | 4.8 | 6.1 | (0.14, 0.05) | 74 |
| Example 27 | 484 | 4.8 | 6.3 | (0.14, 0.05) | 72 |
| Example 28 | 491 | 4.6 | 6.2 | (0.14, 0.05) | 68 |
| Comparative | NPB | 5.5 | 5.7 | (0.14, 0.05) | 56 |
| Example 1 | | | | | |
| Comparative | H1 | 5.4 | 5.5 | (0.14, 0.05) | 52 |
| Example 2 | | | | | |
| Comparative | H2 | 5.3 | 5.7 | (0.14, 0.05) | 58 |
| Example 3 | | | | | |
| Comparative | H3 | 5.3 | 5.6 | (0.14, 0.05) | 55 |
| Example 4 | | | | | |
| Comparative | H4 | 5.3 | 5.7 | (0.14, 0.05) | 4 |
| Example 5 | | | | | |
| Comparative | H5 | 5.4 | 5.5 | (0.14, 0.05) | 57 |
| Example 6 | | | | | |
| Comparative | H6 | 5.5 | 5.5 | (0.14, 0.05) | 53 |
| Example 7 | | | | | |
| Comparative | H7 | 5.6 | 5.4 | (0.14, 0.05) | 51 |
| Example 8 | | | | | |
| Comparative | H8 | 5.5 | 5.6 | (0.14, 0.05) | 48 |
| Example 9 | | | | | |

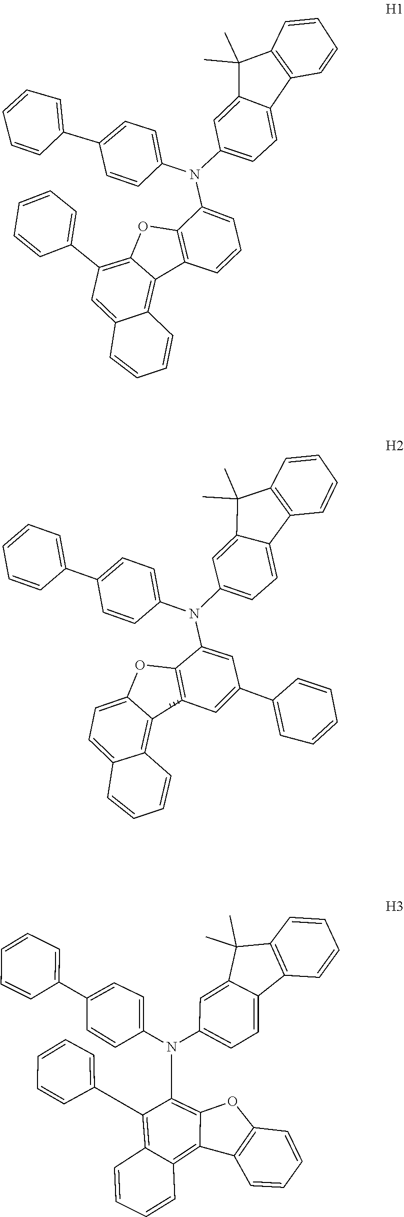

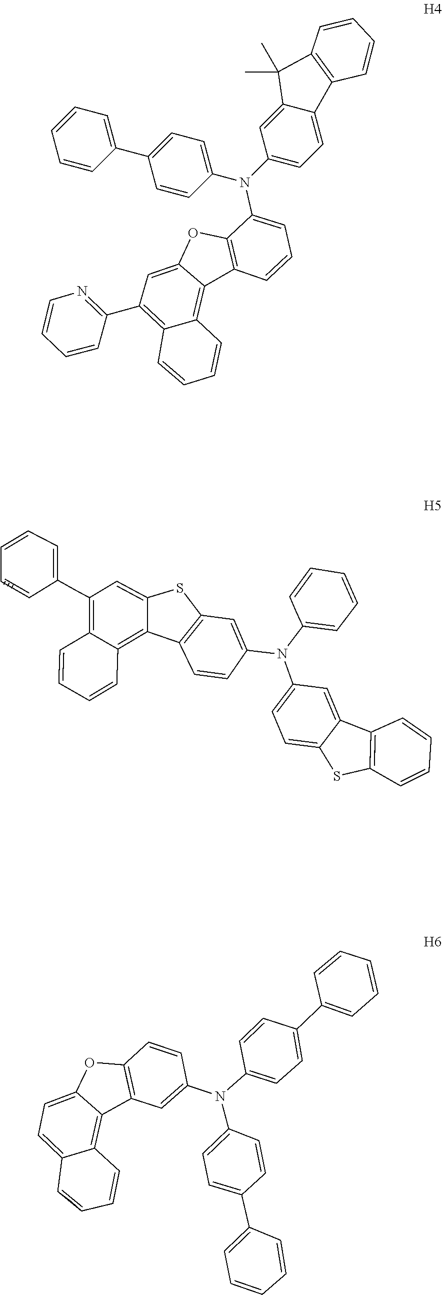

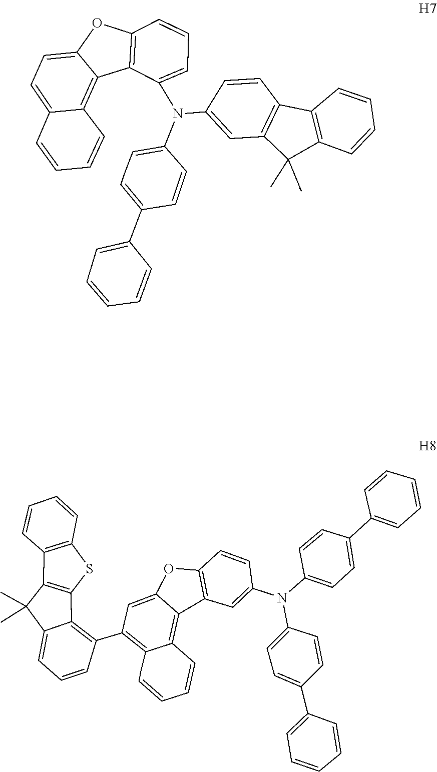 [SMILES: CCC1(C)c2ccccc2-c2ccc(N(C3=CCC=C(c4ccccc4)C=C3)c3cccc4oc5ccc6ccccc6c5c34)cc21 ; CC1(C)c2cccc(-c3cc4oc5ccc(N(c6ccc(-c7ccccc7)cc6)c6ccc(-c7ccccc7)cc6)cc5c4c4c3CC=CC=C4)c2-c2sc3ccccc3c21]

As can be seen from the results in Table 5, the organic light emitting device using a hole transport layer material of the blue organic light emitting device of the present invention has a low driving voltage and remarkably improved light emitting efficiency and service life compared to Comparative Examples 1 to 9. In the case of naphtho[2,1-b]benzofuran compounds with substituents at appropriate positions, it is possible to prevent the deterioration of device characteristics by suppressing the pi-pi stacking of aromatic rings to improve device stability. Therefore, it can be seen that the compound of the present invention using these derivatives brings excellence in terms of all the aspects in driving, efficiency, and service life.

Specifically, it could be seen that the compounds used in Comparative Examples 2 to 4 have different substituent positions from Compound 41 used in Example 3, and in the case of Comparative Examples 2 to 4, in which the substituent positions do not satisfy Chemical Formula 1 of the present invention, the driving voltage was higher, the light emitting efficiency was lower, and the service life was shorter than those of Example 3.

Further, Compound H5 used in Comparative Example 5 includes a naphthobenzothiophene structure as a core structure, instead of naphthobenzofuran, and it could be confirmed that the compound showed lower performance in terms of driving voltage, light emitting efficiency, and service life than the case of using Compound 111 in Example 5, which has naphthobenzofuran as a core structure.

For Compounds H6 and H7 of Comparative Examples 6 and 7, a core structure of the present invention has only one substituent, and it could be seen that performance deteriorates in all aspects when the driving voltage, light emitting efficiency, and service life of the compounds are compared with those of Examples 12 and 18, respectively.

In particular, in the case of Comparative Example 5 (a pyrimidine group) and Comparative Example 9 (a tetracyclic heteroaryl group) in which the characteristics of the substituent Ar are different from those of Chemical Formula 1 of the present invention, it could be confirmed that the service life was remarkably reduced compared to Examples 3 and 12, respectively.

### Experimental Example 2

### 1) Manufacture of Organic Light Emitting Device

### Comparative Example 10

Trichloroethylene, acetone, ethanol, and distilled water were each sequentially used to ultrasonically wash a transparent electrode indium tin oxide (ITO) thin film obtained from glass for OLED (manufactured by Samsung-Corning Co., Ltd.) for 5 minutes, and then the ITO thin film was placed in isopropanol, stored, and then used. Next, the ITO substrate was disposed in a substrate folder of a vacuum deposition apparatus, and the following 4,4′,4″-tris(N,N-(2-naphthyl)-phenylamino)triphenyl amine (2-TNATA) was placed in a cell in the vacuum deposition apparatus.

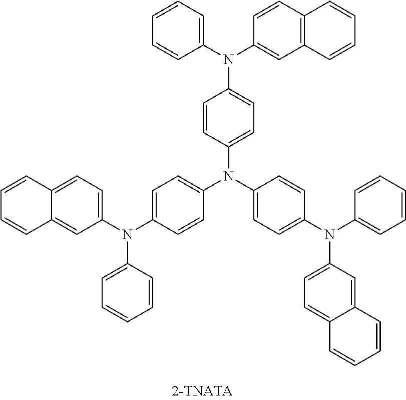 [SMILES: c1ccc(N(c2ccc(N(c3ccc(N(c4ccccc4)c4ccc5ccccc5c4)cc3)c3ccc(N(c4ccccc4)c4ccc5ccccc5c4)cc3)cc2)c2ccc3ccccc3c2)cc1]

Subsequently, air in the chamber was evacuated until the degree of vacuum in the chamber reached 10⁻⁶ torr, and then a hole injection layer having a thickness of 600 Å was deposited on the ITO substrate by applying current to the cell to evaporate 2-TNATA. A hole transport layer having a thickness of 300 Å was deposited on the hole injection layer by placing the following N,N′-bis(α-naphthyl)—N,N′-diphenyl-4,4′-diamine (NPB) in another cell in the vacuum deposition apparatus and applying current to the cell to evaporate NPB.

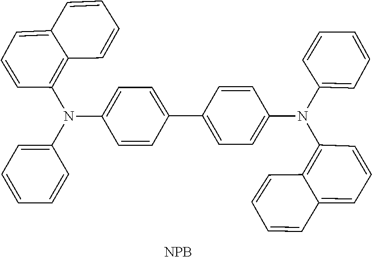 [SMILES: c1ccc(N(c2ccc(-c3ccc(N(c4ccccc4)c4cccc5ccccc45)cc3)cc2)c2cccc3ccccc23)cc1 ; "NPB"]

The hole injection layer and the hole transport layer were formed as described above, and then a blue light emitting material having the following structure as a light emitting layer was deposited thereon. Specifically, a blue light emitting host material BH1 was vacuum deposited to have a thickness of 200 Å on one cell in the vacuum deposition apparatus, and a blue light emitting dopant material D1 was vacuum deposited thereon in an amount of 5% based on the host material.

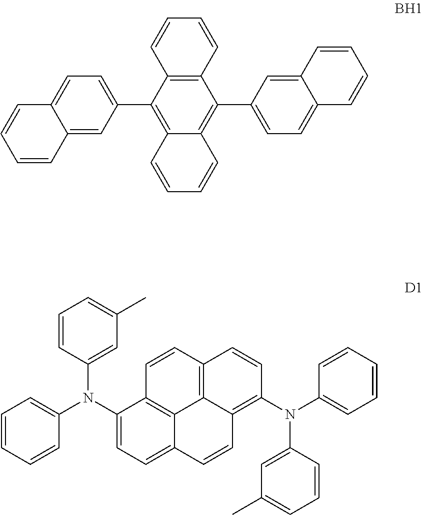

Subsequently, a compound having the following structural formula E1 as an electron transport layer was deposited to have a thickness of 300 Å.

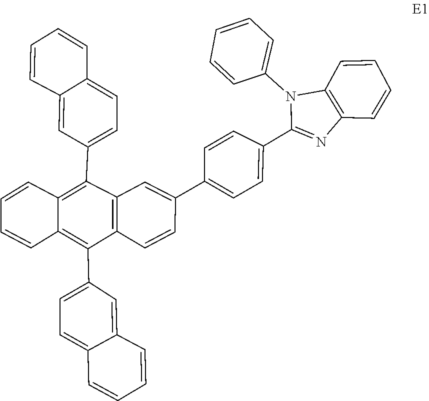 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1]

An OLED device was manufactured by depositing lithium fluoride (LiF) as an electron injection layer to have a thickness of 10 Λ and allowing the Al negative electrode to have a thickness of 1,000 Å.

Meanwhile, all the organic compounds required for manufacturing an OLED device were subjected to vacuum sublimed purification under 10⁻⁶ to 10⁻⁸ torr for each material, and used for the manufacture of OLED.

### Examples 29 to 56 and Comparative Examples 11 to 18

An organic electroluminescent device was manufactured in the same manner as in Experimental Example 2, except that a hole transport layer (NPB) was formed to a thickness of 250 Å, and then an electron blocking layer was formed to a thickness of 50 Å on the upper portion of the hole transport layer using the compound shown in the following Table 6 in Experimental Example 2.

### 2) Performance Evaluation of Organic Light Emitting Device

For the organic electroluminescence device manufactured as described above, electroluminescence (EL) characteristics were measured by M7000 manufactured by McScience Inc., and based on the measurement result thereof, T₉₅ was measured by a service life measurement device (M6000) manufactured by McScience Inc., when the reference luminance was 6,000 cd/m².

The results of measuring the driving voltage, light emitting efficiency, color coordinate (CIE) and service life of the blue organic light emitting device manufactured according to the present invention are shown in the following Table 6.

TABLE 6

| | | | Light | | |
|---|---|---|---|---|---|
| | | Driving | emitting | | Service |
| | | voltage | efficiency | CIE | life |
| | Compound | (V) | (cd/A) | (x, y) | (T₉₅) |
| Example 29 | 4 | 4.7 | 6.1 | (0.14, 0.05) | 79 |
| Example 30 | 25 | 4.5 | 6.3 | (0.14, 0.05) | 71 |
| Example 31 | 41 | 4.6 | 6.2 | (0.14, 0.05) | 82 |
| Example 32 | 65 | 4.6 | 6.2 | (0.14, 0.05) | 70 |
| Example 33 | 93 | 4.5 | 6.4 | (0.14, 0.05) | 77 |
| Example 34 | 111 | 4.7 | 6.3 | (0.14, 0.05) | 68 |
| Example 35 | 135 | 4.5 | 6.4 | (0.14, 0.05) | 71 |
| Example 36 | 157 | 4.6 | 6.4 | (0.14, 0.05) | 80 |
| Example 37 | 178 | 4.7 | 6.1 | (0.14, 0.05) | 78 |
| Example 38 | 185 | 4.7 | 6.2 | (0.14, 0.05) | 73 |
| Example 39 | 207 | 4.7 | 6.2 | (0.14, 0.05) | 77 |
| Example 40 | 233 | 4.7 | 6.3 | (0.14, 0.05) | 78 |
| Example 41 | 250 | 4.6 | 6.4 | (0.14, 0.05) | 68 |
| Example 42 | 267 | 4.6 | 6.3 | (0.14, 0.05) | 71 |
| Example 43 | 291 | 4.7 | 6.2 | (0.14, 0.05) | 81 |
| Example 44 | 320 | 4.7 | 6.2 | (0.14, 0.05) | 80 |
| Example 45 | 338 | 4.5 | 6.4 | (0.14, 0.05) | 82 |
| Example 46 | 341 | 4.6 | 6.2 | (0.14, 0.05) | 77 |
| Example 47 | 368 | 4.5 | 6.2 | (0.14, 0.05) | 72 |
| Example 48 | 397 | 4.7 | 6.2 | (0.14, 0.05) | 78 |
| Example 49 | 402 | 4.7 | 6.3 | (0.14, 0.05) | 74 |
| Example 50 | 426 | 4.7 | 6.4 | (0.14, 0.05) | 70 |
| Example 51 | 449 | 4.6 | 6.2 | (0.14, 0.05) | 68 |
| Example 52 | 480 | 4.6 | 6.1 | (0.14, 0.05) | 80 |
| Example 53 | 481 | 4.6 | 6.4 | (0.14, 0.05) | 84 |
| Example 54 | 483 | 4.7 | 6.2 | (0.14, 0.05) | 89 |
| Example 55 | 484 | 4.7 | 6.3 | (0.14, 0.05) | 87 |
| Example 56 | 491 | 4.7 | 6.3 | (0.14, 0.05) | 72 |
| Comparative | NPB | 5.4 | 5.7 | (0.14, 0.05) | 58 |
| Example 10 | | | | | |
| Comparative | H1 | 5.3 | 5.6 | (0.14, 0.05) | 54 |
| Example 11 | | | | | |
| Comparative | H2 | 5.2 | 5.7 | (0.14, 0.05) | 59 |
| Example 12 | | | | | |
| Comparative | H3 | 5.2 | 5.6 | (0.14, 0.05) | 56 |
| Example 13 | | | | | |
| Comparative | H4 | 5.2 | 5.7 | (0.14, 0.05) | 5 |
| Example 14 | | | | | |
| Comparative | H5 | 5.3 | 5.6 | (0.14, 0.05) | 58 |
| Example 15 | | | | | |
| Comparative | H6 | 5.4 | 5.5 | (0.14, 0.05) | 55 |
| Example 16 | | | | | |
| Comparative | H7 | 5.5 | 5.5 | (0.14, 0.05) | 52 |
| Example 17 | | | | | |
| Comparative | H8 | 5.4 | 5.6 | (0.14, 0.05) | 49 |
| Example 18 | | | | | |

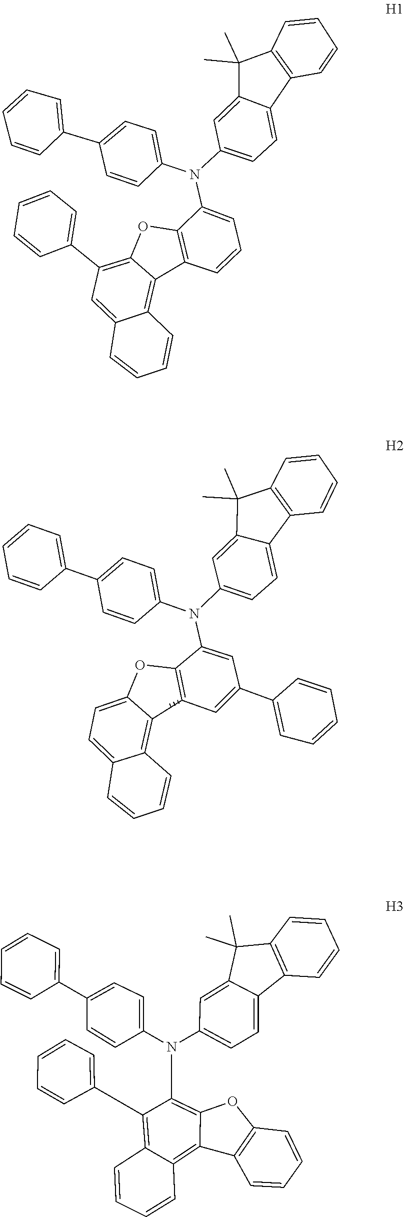

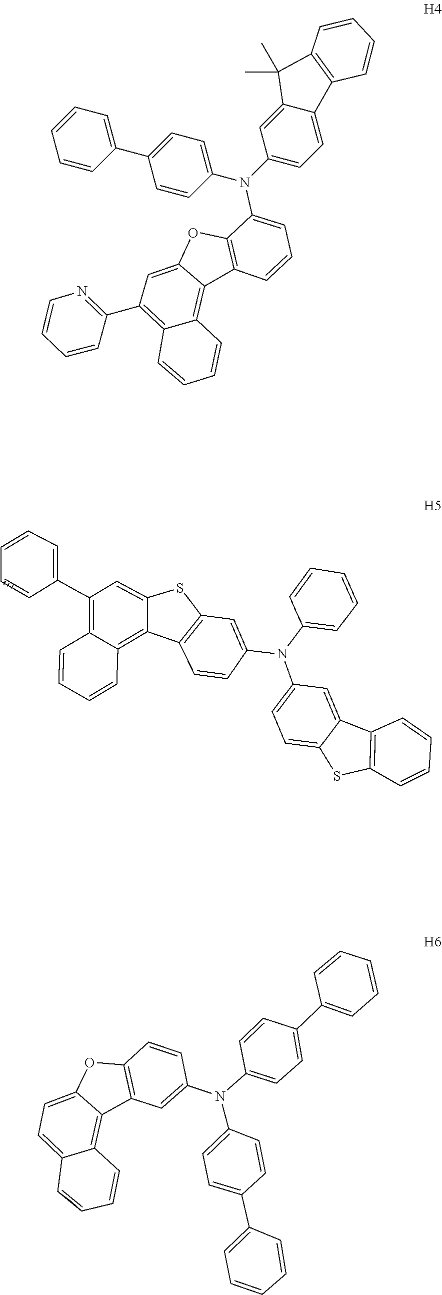

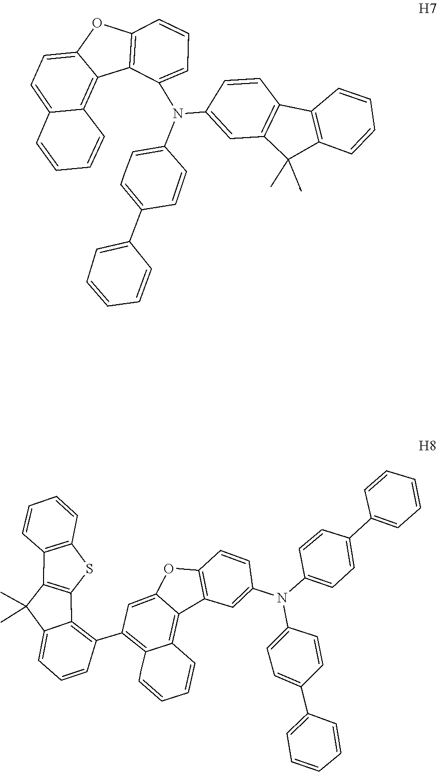 [SMILES: CCC1(C)c2ccccc2-c2ccc(N(C3=CCC=C(c4ccccc4)C=C3)c3cccc4oc5ccc6ccccc6c5c34)cc21 ; CC1(C)c2cccc(-c3cc4oc5ccc(N(c6ccc(-c7ccccc7)cc6)c6ccc(-c7ccccc7)cc6)cc5c4c4c3CC=CC=C4)c2-c2sc3ccccc3c21]

As can be seen from the results in Table 6, the organic light emitting device using an electron blocking layer material of the blue organic light emitting device of the present invention has a low driving voltage and improved light emitting efficiency and service life compared to the Comparative Examples. When the electrons are not combined in the light emitting layer and pass through the hole transport layer and go to the positive electrode, there occurs a phenomenon in which the efficiency and service life of the OLED device are reduced. When a compound with a high LUMO level is used as an electron blocking layer to prevent such a phenomenon, electrons trying to pass through the light emitting layer to the positive electrode are blocked by the energy barrier of the electron blocking layer. Therefore, it could be seen that the probability of holes and electrons forming excitons is increased, and the excitons are highly likely to be emitted from the light-emitting layer as light, and thus the compounds of the present invention brought excellence in terms of all the aspects in driving, efficiency, and service life.

Specifically, it could be seen that the compounds used in Comparative Examples 11 to 13 have different substituent positions from Compound 41 used in Example 31, and in the case of Comparative Examples 11 to 13, in which the substituent positions do not satisfy Chemical Formula 1 of the present invention, the driving voltage is higher, the light emitting efficiency is lower than those of Example 31, and the service life is also short, at 70% of that of Example 31.

Further, Compound H5 used in Comparative Example 15 includes a naphthobenzothiophene structure as a core structure, instead of naphthobenzofuran, and it can be confirmed that the compound shows lower performance in terms of all the aspects in driving voltage, light emitting efficiency, and service life than the case of using Compound 111 in Example 34, which has naphthobenzofuran as a core structure.

For Compounds H6 and H7 of Comparative Examples 16 and 17, a core structure of the present invention has only one substituent, and it can be seen that performance deteriorates in all aspects when the driving voltage, light emitting efficiency, and service life of the compounds are compared with those of Examples 40 and 46, respectively.

In particular, in the case of Comparative Example 14 (a pyrimidine group) and Comparative Example 18 (a tetracyclic heteroaryl group) in which the characteristics of the substituent Ar are different from those of Chemical Formula 1 of the present invention, it can be confirmed that the service life is remarkably reduced compared to Examples 31 and 40, respectively.

Furthermore, by comparing Example 40 and Example 54, it can be seen that when the compound of Chemical Formula 1 including deuterium compared to the same structure is used as an electron blocking layer material, the service life is improved by about 14%.

### Experimental Example 3

### 1) Manufacture of Organic Light Emitting Device

A glass substrate, in which indium tin oxide (ITO) was thinly coated to have a thickness of 1,500 Å, was ultrasonically washed with distilled water. When the washing with distilled water was finished, the glass substrate was ultrasonically washed with a solvent such as acetone, methanol, and isopropyl alcohol, dried and then subjected to ultraviolet ozone (UVO) treatment for 5 minutes using UV in an ultraviolet (UV) washing machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

A hole injection layer 4,4′,4″-tris[2-naphthyl(phenyl)amino]triphenylamine (2-TNATA), a hole transport layer N,N′-diphenyl-(1,1′-biphenyl)-4,4′-diamine (NPB) and an electron blocking layer cyclohexylidenebis[N,N-bis(4-methylphenyl)benzenamine](TAPC), which are common layers, were formed on the ITO transparent electrode (positive electrode). A light emitting layer was thermally vacuum deposited thereon as follows. The light emitting layer was deposited to have a thickness of 400 Å by depositing two types of the compounds described in the following Table 7 as a red host from a single supply source and doping the host with an Ir compound at 3 wt % using (piq)2(Ir) (acac) as a red phosphorescent dopant. In the following Table 7, P means a P-type host and N means an N-type host.

Thereafter, Bphen as a hole blocking layer was deposited to have a thickness of 30 Å, and TPBI as an electron transport layer was deposited to have a thickness of 250 Å thereon. Finally, lithium fluoride (LiF) was deposited to have a thickness of 10 Å on the electron transport layer to form an electron injection layer, and then aluminum (Al) negative electrode was deposited to have a thickness of 1,200 Å on the electron injection layer to form a negative electrode, thereby manufacturing an organic electroluminescence device.

Meanwhile, all the organic compounds required for manufacturing an OLED device were subjected to vacuum sublimed purification under 10⁻⁶ to 10⁻⁸ torr for each material, and used for the manufacture of OLED.

### 2) Performance Evaluation of Organic Light Emitting Device

For the organic electroluminescence device manufactured as described above, electroluminescence (EL) characteristics were measured by M7000 manufactured by McScience Inc., and based on the measurement result thereof, T₉₀ was measured by a service life measurement device (M6000) manufactured by McScience Inc., when the reference luminance was 6,000 cd/m².

The results of measuring the driving voltage, light emitting efficiency, color coordinate (CIE) and service life of the organic light emitting device manufactured according to the present invention are shown in Table 7.

TABLE 7

| | | | | Light | | |
|---|---|---|---|---|---|---|
| | | | Driving | emitting | | Service |
| | Compound | Ratio | voltage | efficiency | CIE | life |
| | (P:N) | (P:N) | (V) | (cd/A) | (x, y) | (T₉₀) |
| Example 57 | 4:NH9 | 1:3 | 4.2 | 53.3 | (0.68, 0.32) | 147 |
| Example 58 | 4:NH9 | 1:2 | 4.2 | 53.7 | (0.68, 0.32) | 147 |
| Example 59 | 4:NH9 | 1:1 | 4.2 | 55.2 | (0.68, 0.32) | 150 |
| Example 60 | 4:NH9 | 2:1 | 4.4 | 51.5 | (0.68, 0.32) | 131 |
| Example 61 | 4:NH9 | 3:1 | 4.6 | 49.1 | (0.68, 0.32) | 123 |
| Example 62 | 25:NH9 | 1:1 | 4.2 | 56.3 | (0.68, 0.32) | 135 |
| Example 63 | 41:NH9 | 1:1 | 4.3 | 57.1 | (0.68, 0.32) | 130 |
| Example 64 | 65:NH57 | 1:1 | 4.4 | 55.5 | (0.68, 0.32) | 149 |
| Example 65 | 93:NH57 | 1:1 | 4.1 | 53.7 | (0.68, 0.32) | 144 |
| Example 66 | 111:NH57 | 1:1 | 4.5 | 54.8 | (0.68, 0.32) | 138 |
| Example 67 | 135:NH84 | 1:1 | 4.2 | 52.9 | (0.68, 0.32) | 155 |
| Example 68 | 157:NH84 | 1:1 | 4.3 | 51.5 | (0.68, 0.32) | 147 |
| Example 69 | 178:NH84 | 1:1 | 4.3 | 53.7 | (0.68, 0.32) | 139 |
| Example 70 | 185:NH131 | 1:1 | 4.1 | 56.0 | (0.68, 0.32) | 164 |
| Example 71 | 207:NH131 | 1:1 | 4.4 | 51.9 | (0.68, 0.32) | 147 |
| Example 72 | 233:NH131 | 1:1 | 4.5 | 52.4 | (0.68, 0.32) | 158 |
| Example 73 | 250:NH141 | 1:1 | 4.0 | 52.9 | (0.68, 0.32) | 144 |
| Example 74 | 267:NH141 | 1:1 | 4.1 | 54.3 | (0.68, 0.32) | 137 |
| Example 75 | 291:NH141 | 1:1 | 4.2 | 55.2 | (0.68, 0.32) | 153 |
| Example 76 | 320NH48 | 1:1 | 4.3 | 53.7 | (0.68, 0.32) | 142 |
| Example 77 | 338:NH48 | 1:1 | 4.5 | 51.4 | (0.68, 0.32) | 152 |
| Example 78 | 341:NH48 | 1:1 | 4.1 | 51.9 | (0.68, 0.32) | 160 |
| Example 79 | 368:NH9 | 1:1 | 4.3 | 53.8 | (0.68, 0.32) | 142 |
| Example 80 | 397:NH9 | 1:1 | 4.4 | 54.7 | (0.68, 0.32) | 151 |
| Example 81 | 402:NH9 | 1:1 | 4.5 | 56.3 | (0.68, 0.32) | 144 |
| Example 82 | 426:NH115 | 1:1 | 4.0 | 55.3 | (0.68, 0.32) | 133 |
| Example 83 | 449:NH115 | 1:1 | 4.1 | 53.8 | (0.68, 0.32) | 152 |
| Example 84 | 480:NH115 | 1:1 | 4.3 | 54.6 | (0.68, 0.32) | 146 |
| Example 85 | 481:NH121 | 1:1 | 4.5 | 52.7 | (0.68, 0.32) | 160 |
| Example 86 | 483:NH121 | 1:1 | 4.1 | 54.4 | (0.68, 0.32) | 165 |
| Example 87 | 484:NH121 | 1:1 | 4.3 | 55.9 | (0.68, 0.32) | 169 |
| Example 88 | 491:NH9 | 1:1 | 4.5 | 53.3 | (0.68, 0.32) | 154 |
| Comparative | H1:NH9 | 1:1 | 5.0 | 45.9 | (0.68, 0.32) | 85 |
| Example 19 | | | | | | |
| Comparative | H2:NH57 | 1:1 | 4.9 | 46.2 | (0.68, 0.32) | 98 |
| Example 20 | | | | | | |
| Comparative | H3:NH57 | 1:1 | 4.9 | 47.4 | (0.68, 0.32) | 90 |
| Example 21 | | | | | | |
| Comparative | H4:NH89 | 1:1 | 4.8 | 48.7 | (0.68, 0.32) | 15 |
| Example 22 | | | | | | |
| Comparative | H5:NH89 | 1:1 | 4.9 | 45.8 | (0.68, 0.32) | 87 |
| Example 23 | | | | | | |
| Comparative | H6:NH131 | 1:1 | 4.8 | 47.0 | (0.68, 0.32) | 94 |
| Example 24 | | | | | | |
| Comparative | H7:NH131 | 1:1 | 5.0 | 45.2 | (0.68, 0.32) | 85 |
| Example 25 | | | | | | |
| Comparative | H8:NH141 | 1:1 | 5.0 | 45.8 | (0.68, 0.32) | 80 |
| Example 26 | | | | | | |

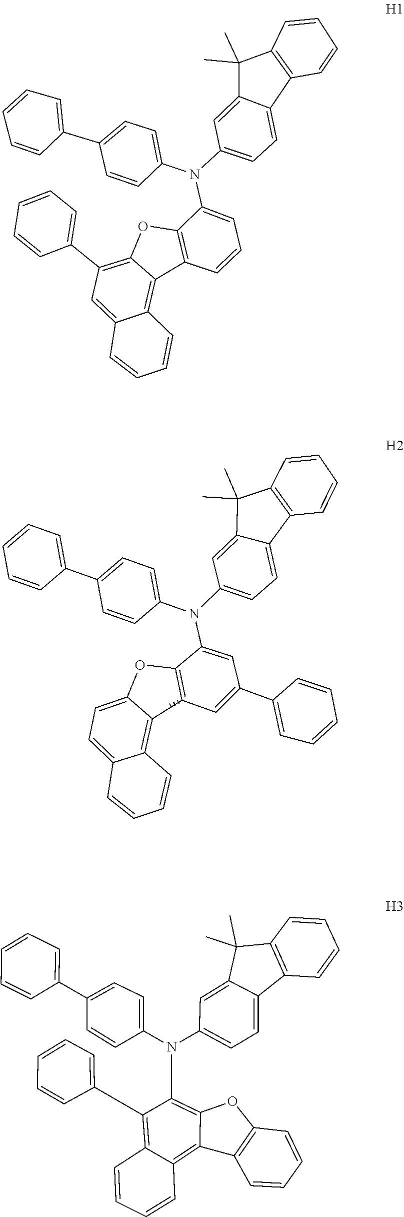

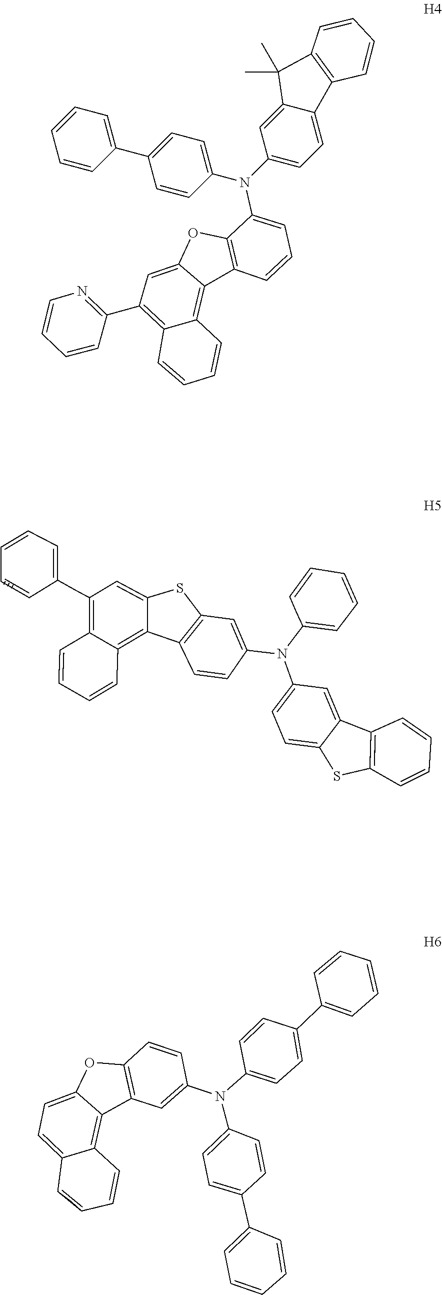

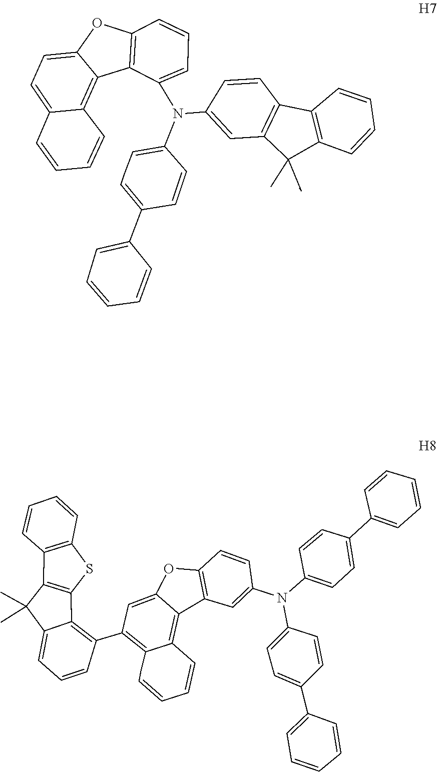 [SMILES: CCC1(C)c2ccccc2-c2ccc(N(C3=CCC=C(c4ccccc4)C=C3)c3cccc4oc5ccc6ccccc6c5c34)cc21 ; CC1(C)c2cccc(-c3cc4oc5ccc(N(c6ccc(-c7ccccc7)cc6)c6ccc(-c7ccccc7)cc6)cc5c4c4c3CC=CC=C4)c2-c2sc3ccccc3c21]

As can be seen from the results in Table 7, it could be confirmed that when the heterocyclic compound of the present invention was used as a P-type host and mixed with an N-type host of Chemical Formula 2 and deposited, the driving, efficiency, and service life of the organic light emitting device were improved. When a donor (p-host) with good hole transporting ability and an acceptor (n-host) with good electron transporting ability are used as hosts of the light emitting layer, the charge balance within the device can be adjusted because holes are injected into the p-host and electrons are injected into the n-host due to the exciplex phenomenon of the N+P compound. From these results, it can be seen that a combination of an N-type host compound with appropriate electron transfer properties and a P-type host compound with appropriate hole transfer properties at an appropriate ratio can help improve driving voltage and service life.

1. A heterocyclic compound of the following Chemical Formula 1: 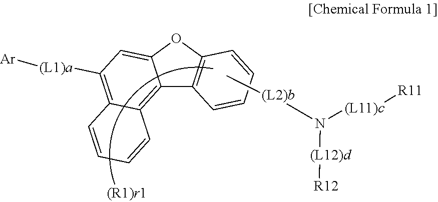
wherein, in Chemical Formula 1,
L1, L2, L11, and L12 are each independently a direct bond; or a substituted or unsubstituted C6 to C60 arylene group,
a, b, c, and d are an integer from 0 to 4,
when a, b, c, and d are 2 or higher, substituents in the parenthesis are the same as or different from each other,
Ar is a substituted or unsubstituted C6 to C60 aryl group; or a substituted or unsubstituted heteroaryl group having up to 3 rings containing O or S,
R1, R11, and R12 are each independently hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, and
r1 is an integer from 0 to 8, and when r1 is 2 or higher, R1's are the same as or different from each other.

2. The heterocyclic compound of claim 1, wherein Chemical Formula 1 is represented by any one of the following Chemical Formulae 1-1 to 1-4: 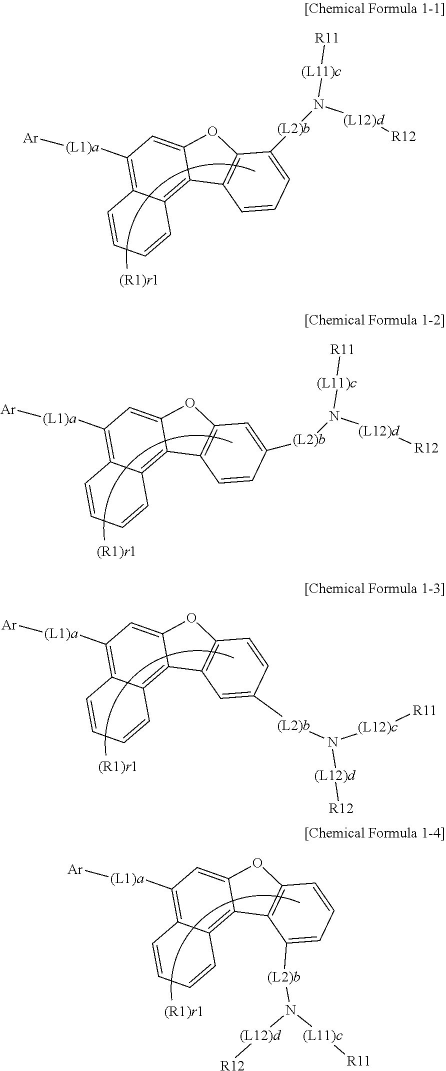
in Chemical Formulae 1-1 to 1-4,
the definition of each substituent is the same as that in Chemical Formula 1.

3. The heterocyclic compound of claim 1, wherein Ar is a substituted or unsubstituted phenyl group; a substituted or unsubstituted biphenyl group; a substituted or unsubstituted terphenyl group; a substituted or unsubstituted naphthyl group; a substituted or unsubstituted dibenzofuran group; or a substituted or unsubstituted dibenzothiophene group.

4. The heterocyclic compound of claim 1, wherein R11 and R12 are each independently a substituted or unsubstituted C6 to C40 aryl group; or a substituted or unsubstituted C2 to C40 heteroaryl group.

5. The heterocyclic compound of claim 1, wherein a deuterium content of Chemical Formula 1 is 0%, or 10% to 100%.

6. The heterocyclic compound of claim 1, wherein Chemical Formula 1 is represented by any one of the following compounds:

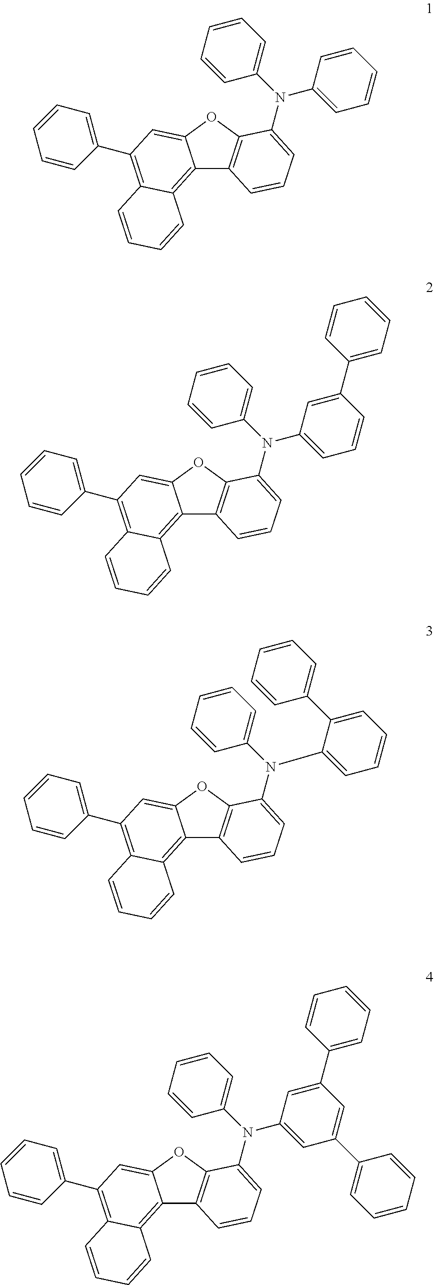 [SMILES: c1ccc(-c2cc3oc4c(N(c5ccccc5)c5ccccc5)cccc4c3c3ccccc23)cc1 ; c1ccc(-c2cccc(N(c3ccccc3)c3cccc4c3oc3cc(-c5ccccc5)c5ccccc5c34)c2)cc1 ; c1ccc(-c2ccccc2N(c2ccccc2)c2cccc3c2oc2cc(-c4ccccc4)c4ccccc4c23)cc1 ; c1ccc(-c2cc(-c3ccccc3)cc(N(c3ccccc3)c3cccc4c3oc3cc(-c5ccccc5)c5ccccc5c34)c2)cc1]

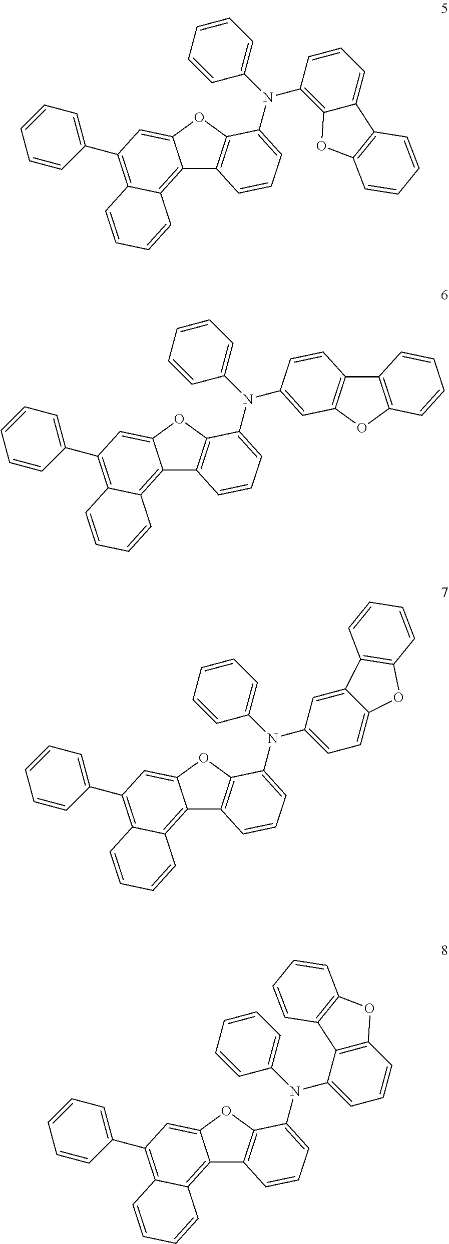 [SMILES: c1ccc(-c2cc3oc4c(N(c5ccccc5)c5cccc6c5oc5ccccc56)cccc4c3c3ccccc23)cc1 ; c1ccc(-c2cc3oc4c(N(c5ccccc5)c5ccc6c(c5)oc5ccccc56)cccc4c3c3ccccc23)cc1 ; c1ccc(-c2cc3oc4c(N(c5ccccc5)c5ccc6oc7ccccc7c6c5)cccc4c3c3ccccc23)cc1 ; c1ccc(-c2cc3oc4c(N(c5ccccc5)c5cccc6oc7ccccc7c56)cccc4c3c3ccccc23)cc1]

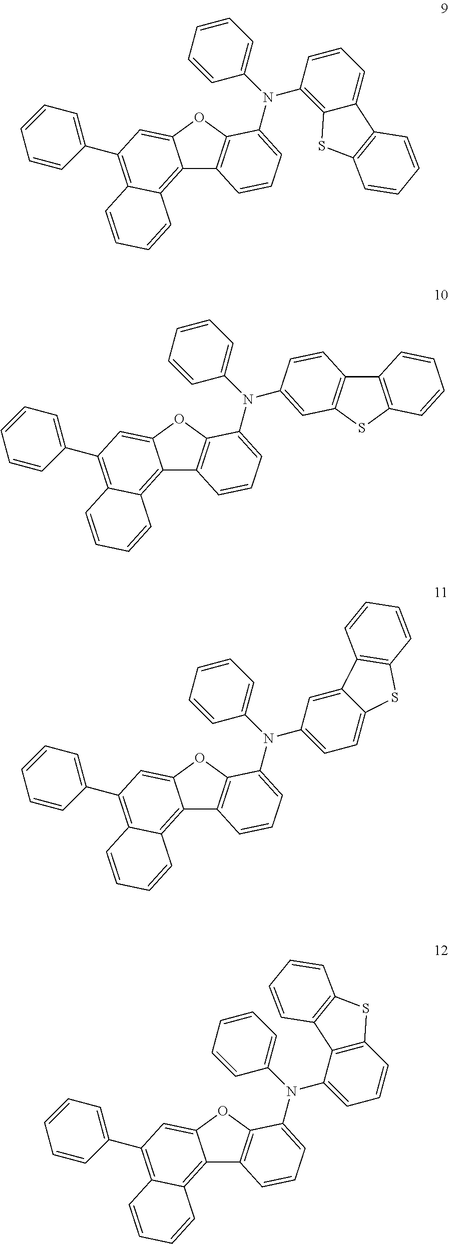 [SMILES: c1ccc(-c2cc3oc4c(N(c5ccccc5)c5cccc6c5sc5ccccc56)cccc4c3c3ccccc23)cc1 ; c1ccc(-c2cc3oc4c(N(c5ccccc5)c5ccc6c(c5)sc5ccccc56)cccc4c3c3ccccc23)cc1 ; c1ccc(-c2cc3oc4c(N(c5ccccc5)c5ccc6sc7ccccc7c6c5)cccc4c3c3ccccc23)cc1 ; c1ccc(-c2cc3oc4c(N(c5ccccc5)c5cccc6sc7ccccc7c56)cccc4c3c3ccccc23)cc1]

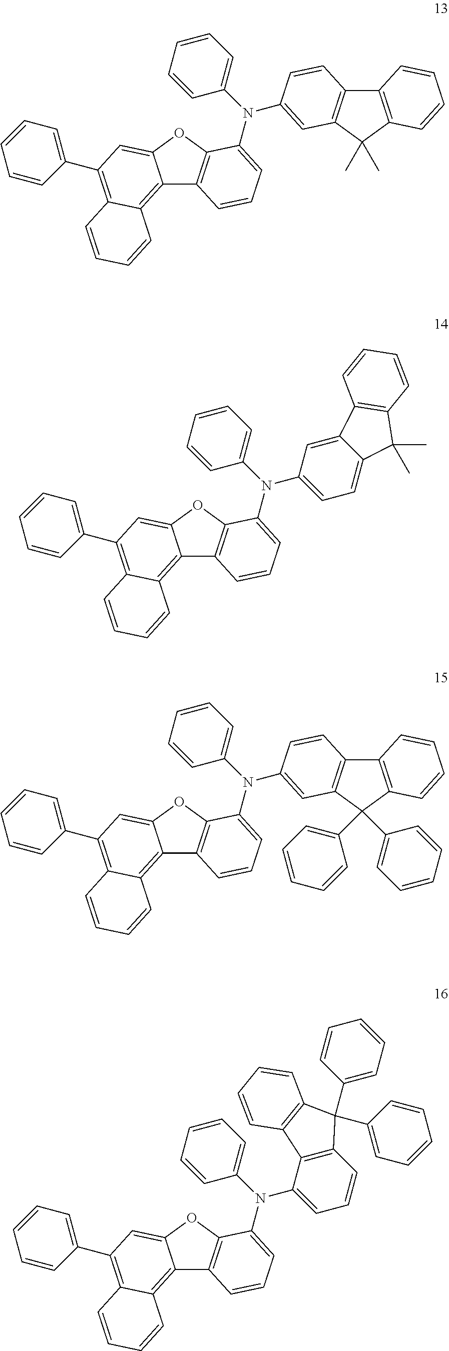 [SMILES: CC1(C)c2ccccc2-c2ccc(N(c3ccccc3)c3cccc4c3oc3cc(-c5ccccc5)c5ccccc5c34)cc21 ; CC1(C)c2ccccc2-c2cc(N(c3ccccc3)c3cccc4c3oc3cc(-c5ccccc5)c5ccccc5c34)ccc21 ; c1ccc(-c2cc3oc4c(N(c5ccccc5)c5ccc6c(c5)C(c5ccccc5)(c5ccccc5)c5ccccc5-6)cccc4c3c3ccccc23)cc1 ; c1ccc(-c2cc3oc4c(N(c5ccccc5)c5cccc6c5-c5ccccc5C6(c5ccccc5)c5ccccc5)cccc4c3c3ccccc23)cc1]

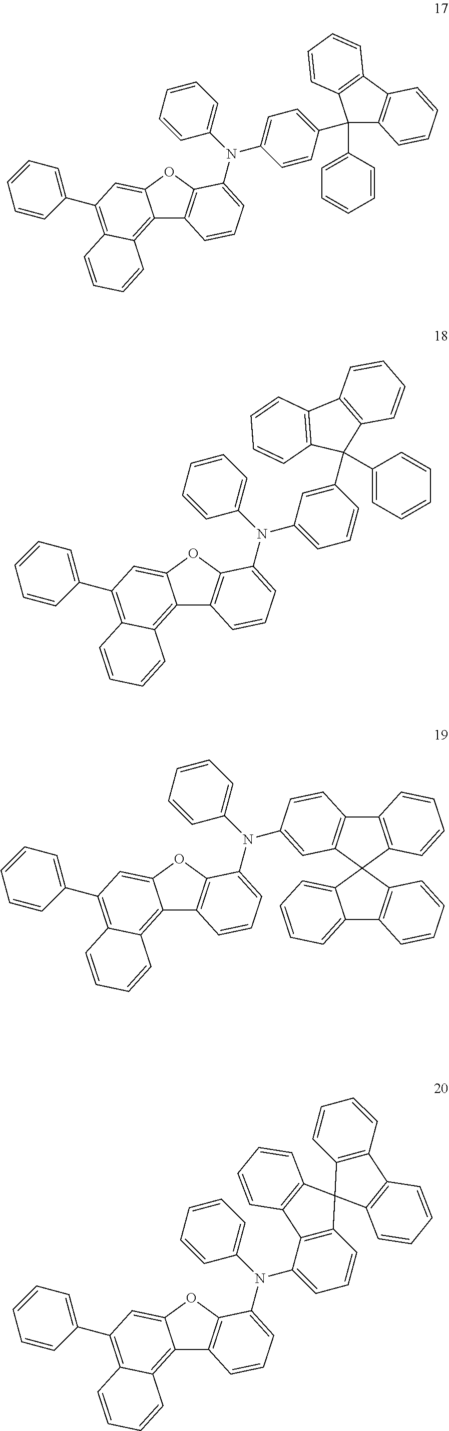 [SMILES: c1ccc(-c2cc3oc4c(N(c5ccccc5)c5ccc(C6(c7ccccc7)c7ccccc7-c7ccccc76)cc5)cccc4c3c3ccccc23)cc1 ; c1ccc(-c2cc3oc4c(N(c5ccccc5)c5cccc(C6(c7ccccc7)c7ccccc7-c7ccccc76)c5)cccc4c3c3ccccc23)cc1 ; c1ccc(-c2cc3oc4c(N(c5ccccc5)c5ccc6c(c5)C5(c7ccccc7-c7ccccc75)c5ccccc5-6)cccc4c3c3ccccc23)cc1 ; c1ccc(-c2cc3oc4c(N(c5ccccc5)c5cccc6c5-c5ccccc5C65c6ccccc6-c6ccccc65)cccc4c3c3ccccc23)cc1]

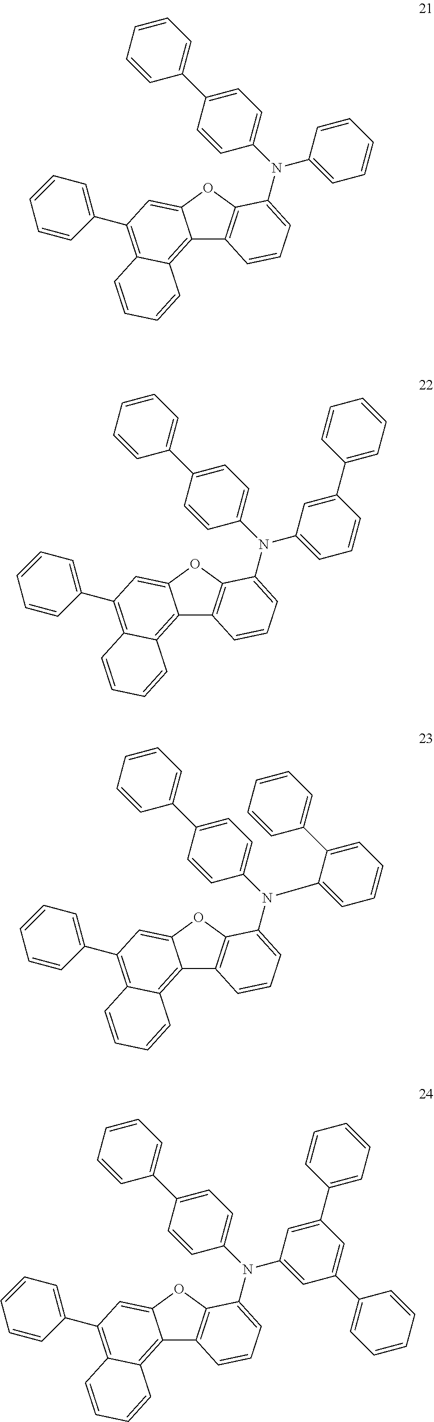 [SMILES: c1ccc(-c2ccc(N(c3ccccc3)c3cccc4c3oc3cc(-c5ccccc5)c5ccccc5c34)cc2)cc1 ; c1ccc(-c2ccc(N(c3cccc(-c4ccccc4)c3)c3cccc4c3oc3cc(-c5ccccc5)c5ccccc5c34)cc2)cc1 ; c1ccc(-c2ccc(N(c3ccccc3-c3ccccc3)c3cccc4c3oc3cc(-c5ccccc5)c5ccccc5c34)cc2)cc1 ; c1ccc(-c2ccc(N(c3cc(-c4ccccc4)cc(-c4ccccc4)c3)c3cccc4c3oc3cc(-c5ccccc5)c5ccccc5c34)cc2)cc1]

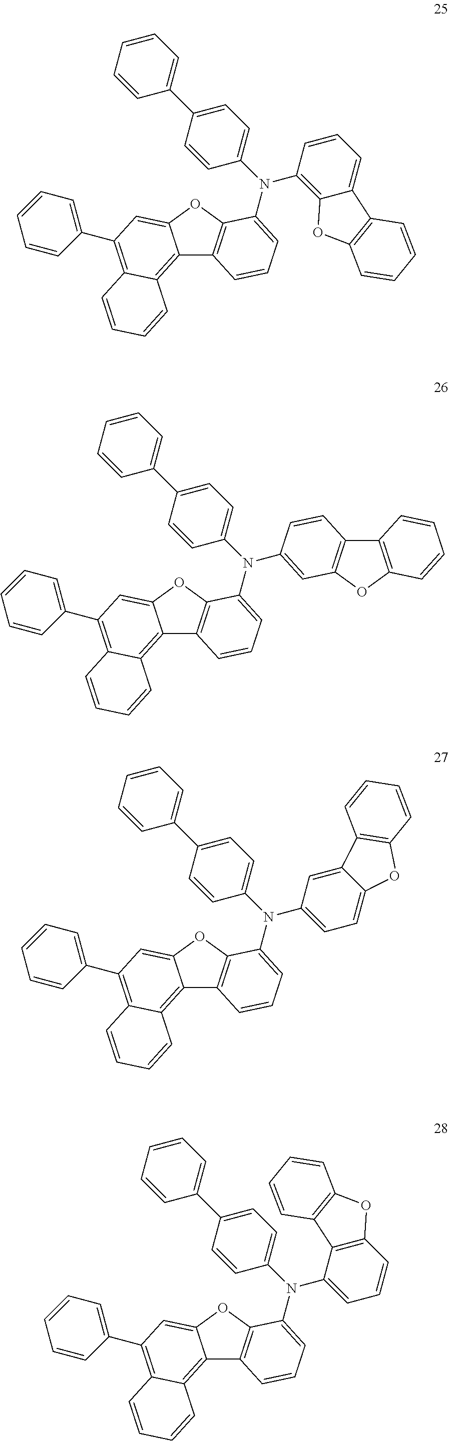 [SMILES: c1ccc(-c2ccc(N(c3cccc4c3oc3ccccc34)c3cccc4c3oc3cc(-c5ccccc5)c5ccccc5c34)cc2)cc1 ; c1ccc(-c2ccc(N(c3ccc4c(c3)oc3ccccc34)c3cccc4c3oc3cc(-c5ccccc5)c5ccccc5c34)cc2)cc1 ; c1ccc(-c2ccc(N(c3ccc4oc5ccccc5c4c3)c3cccc4c3oc3cc(-c5ccccc5)c5ccccc5c34)cc2)cc1 ; c1ccc(-c2ccc(N(c3cccc4c3oc3cc(-c5ccccc5)c5ccccc5c34)c3cccc4oc5ccccc5c34)cc2)cc1]

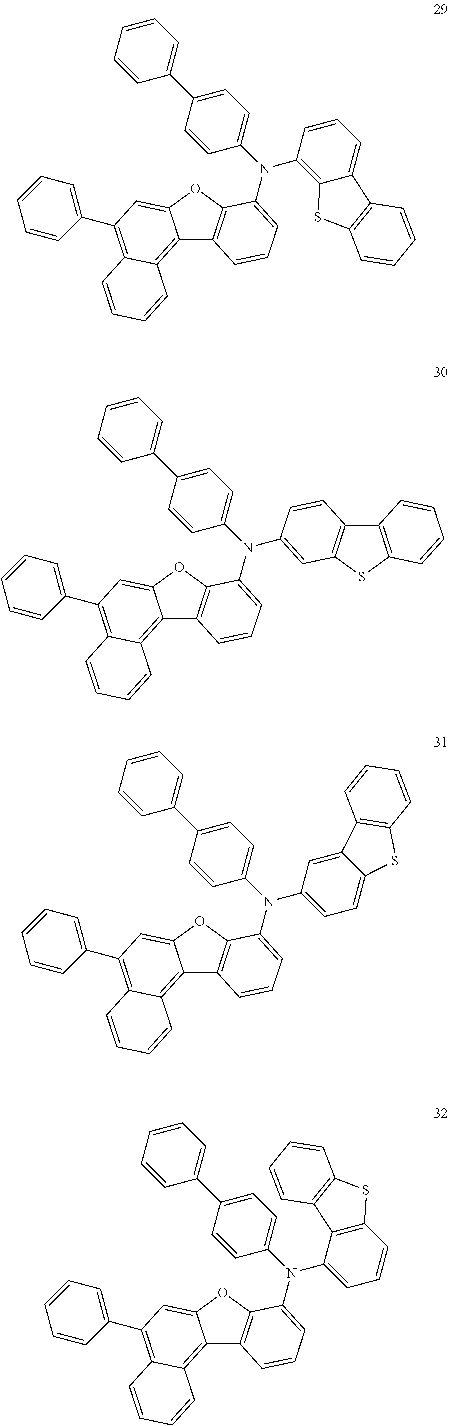 [SMILES: c1ccc(-c2ccc(N(c3cccc4c3oc3cc(-c5ccccc5)c5ccccc5c34)c3cccc4c3sc3ccccc34)cc2)cc1 ; c1ccc(-c2ccc(N(c3ccc4c(c3)sc3ccccc34)c3cccc4c3oc3cc(-c5ccccc5)c5ccccc5c34)cc2)cc1 ; c1ccc(-c2ccc(N(c3ccc4sc5ccccc5c4c3)c3cccc4c3oc3cc(-c5ccccc5)c5ccccc5c34)cc2)cc1 ; c1ccc(-c2ccc(N(c3cccc4c3oc3cc(-c5ccccc5)c5ccccc5c34)c3cccc4sc5ccccc5c34)cc2)cc1]

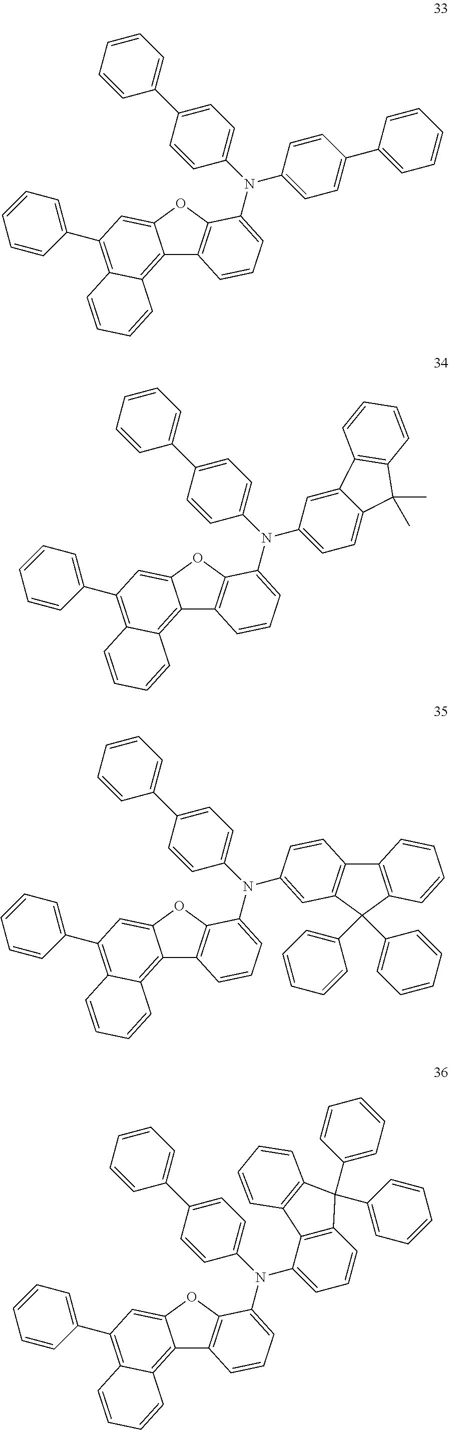 [SMILES: c1ccc(-c2ccc(N(c3ccc(-c4ccccc4)cc3)c3cccc4c3oc3cc(-c5ccccc5)c5ccccc5c34)cc2)cc1 ; CC1(C)c2ccccc2-c2cc(N(c3ccc(-c4ccccc4)cc3)c3cccc4c3oc3cc(-c5ccccc5)c5ccccc5c34)ccc21 ; c1ccc(-c2ccc(N(c3ccc4c(c3)C(c3ccccc3)(c3ccccc3)c3ccccc3-4)c3cccc4c3oc3cc(-c5ccccc5)c5ccccc5c34)cc2)cc1 ; c1ccc(-c2ccc(N(c3cccc4c3-c3ccccc3C4(c3ccccc3)c3ccccc3)c3cccc4c3oc3cc(-c5ccccc5)c5ccccc5c34)cc2)cc1]

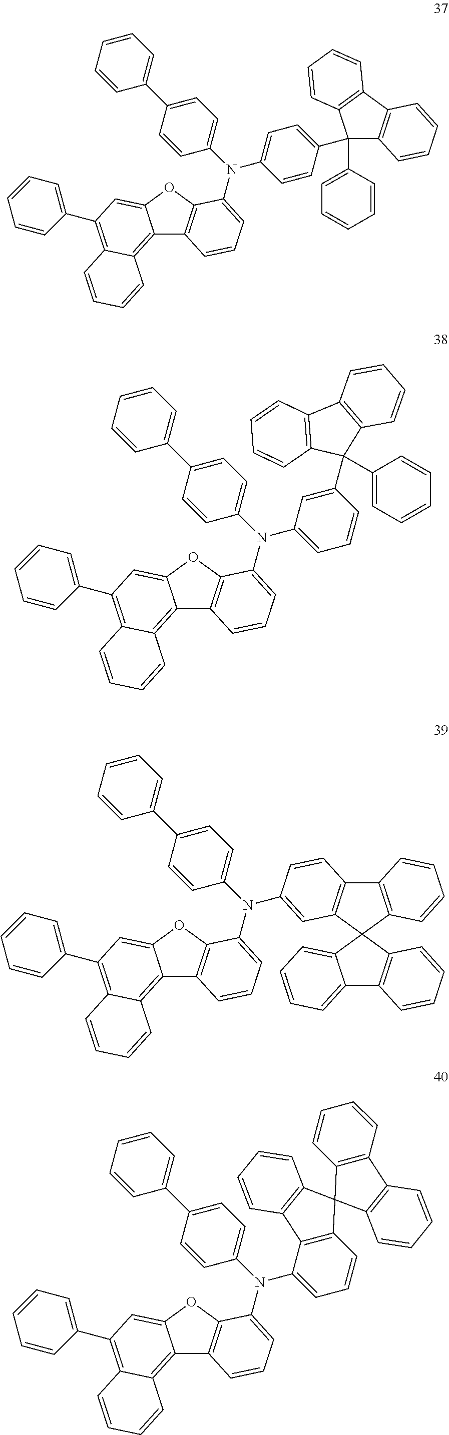 [SMILES: c1ccc(-c2ccc(N(c3ccc(C4(c5ccccc5)c5ccccc5-c5ccccc54)cc3)c3cccc4c3oc3cc(-c5ccccc5)c5ccccc5c34)cc2)cc1 ; c1ccc(-c2ccc(N(c3cccc(C4(c5ccccc5)c5ccccc5-c5ccccc54)c3)c3cccc4c3oc3cc(-c5ccccc5)c5ccccc5c34)cc2)cc1 ; c1ccc(-c2ccc(N(c3ccc4c(c3)C3(c5ccccc5-c5ccccc53)c3ccccc3-4)c3cccc4c3oc3cc(-c5ccccc5)c5ccccc5c34)cc2)cc1 ; c1ccc(-c2ccc(N(c3cccc4c3-c3ccccc3C43c4ccccc4-c4ccccc43)c3cccc4c3oc3cc(-c5ccccc5)c5ccccc5c34)cc2)cc1]

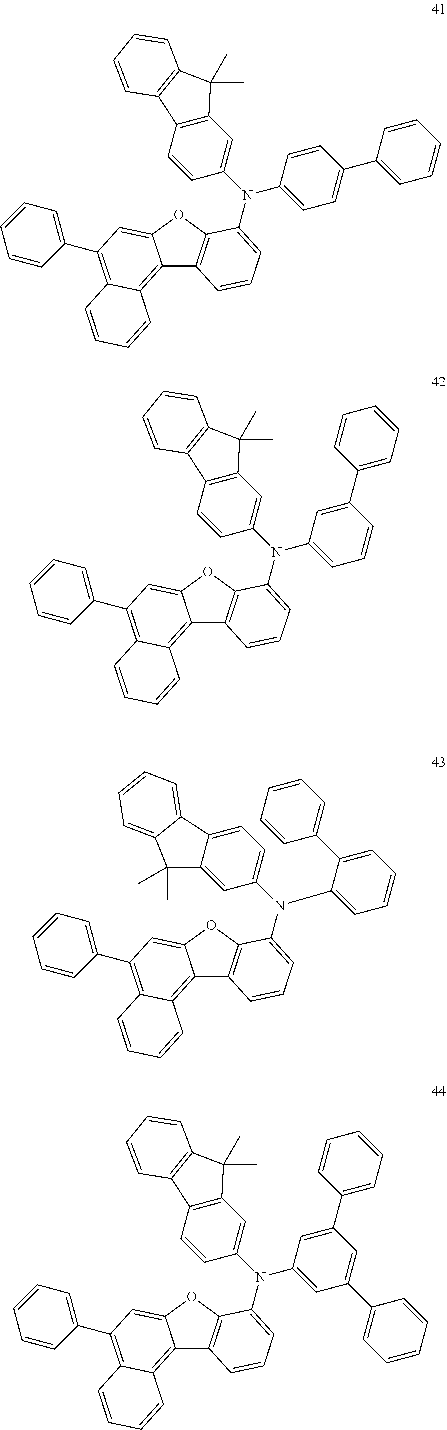 [SMILES: CC1(C)c2ccccc2-c2ccc(N(c3ccc(-c4ccccc4)cc3)c3cccc4c3oc3cc(-c5ccccc5)c5ccccc5c34)cc21 ; CC1(C)c2ccccc2-c2ccc(N(c3cccc(-c4ccccc4)c3)c3cccc4c3oc3cc(-c5ccccc5)c5ccccc5c34)cc21 ; CC1(C)c2ccccc2-c2ccc(N(c3ccccc3-c3ccccc3)c3cccc4c3oc3cc(-c5ccccc5)c5ccccc5c34)cc21 ; CC1(C)c2ccccc2-c2ccc(N(c3cc(-c4ccccc4)cc(-c4ccccc4)c3)c3cccc4c3oc3cc(-c5ccccc5)c5ccccc5c34)cc21]

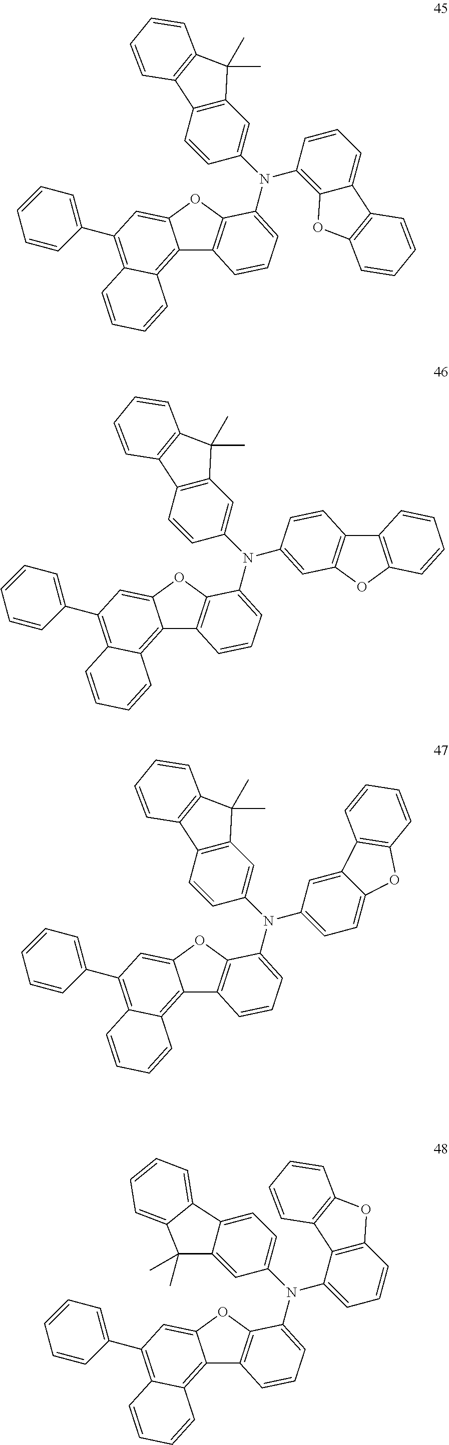 [SMILES: CC1(C)c2ccccc2-c2ccc(N(c3cccc4c3oc3ccccc34)c3cccc4c3oc3cc(-c5ccccc5)c5ccccc5c34)cc21 ; CC1(C)c2ccccc2-c2ccc(N(c3ccc4c(c3)oc3ccccc34)c3cccc4c3oc3cc(-c5ccccc5)c5ccccc5c34)cc21 ; CC1(C)c2ccccc2-c2ccc(N(c3ccc4oc5ccccc5c4c3)c3cccc4c3oc3cc(-c5ccccc5)c5ccccc5c34)cc21 ; CC1(C)c2ccccc2-c2ccc(N(c3cccc4c3oc3cc(-c5ccccc5)c5ccccc5c34)c3cccc4oc5ccccc5c34)cc21]

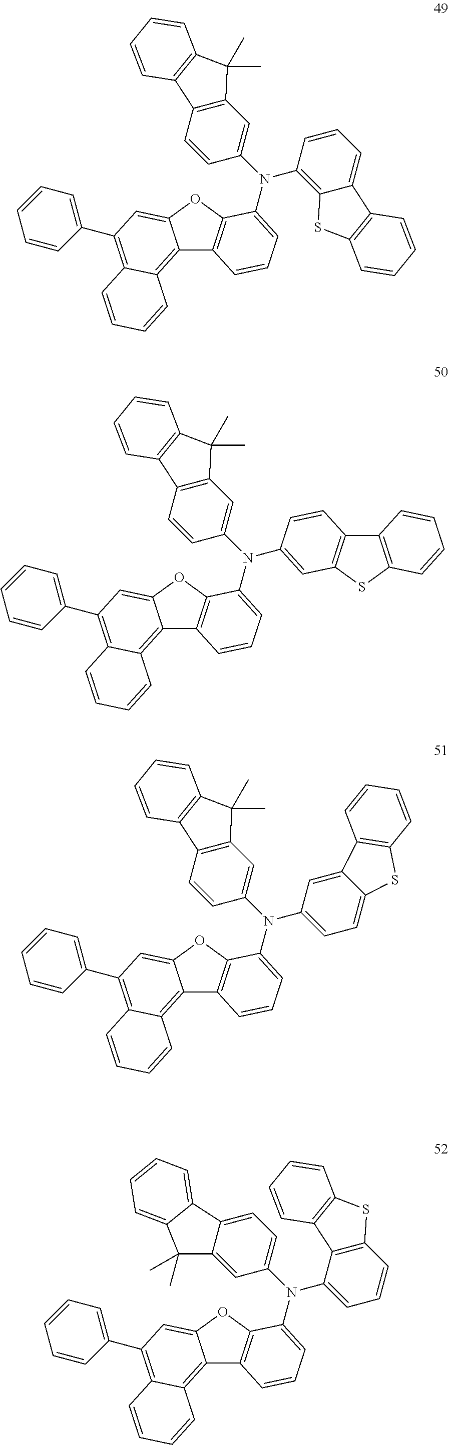 [SMILES: CC1(C)c2ccccc2-c2ccc(N(c3cccc4c3oc3cc(-c5ccccc5)c5ccccc5c34)c3cccc4c3sc3ccccc34)cc21 ; CC1(C)c2ccccc2-c2ccc(N(c3ccc4c(c3)sc3ccccc34)c3cccc4c3oc3cc(-c5ccccc5)c5ccccc5c34)cc21 ; CC1(C)c2ccccc2-c2ccc(N(c3ccc4sc5ccccc5c4c3)c3cccc4c3oc3cc(-c5ccccc5)c5ccccc5c34)cc21 ; CC1(C)c2ccccc2-c2ccc(N(c3cccc4c3oc3cc(-c5ccccc5)c5ccccc5c34)c3cccc4sc5ccccc5c34)cc21]

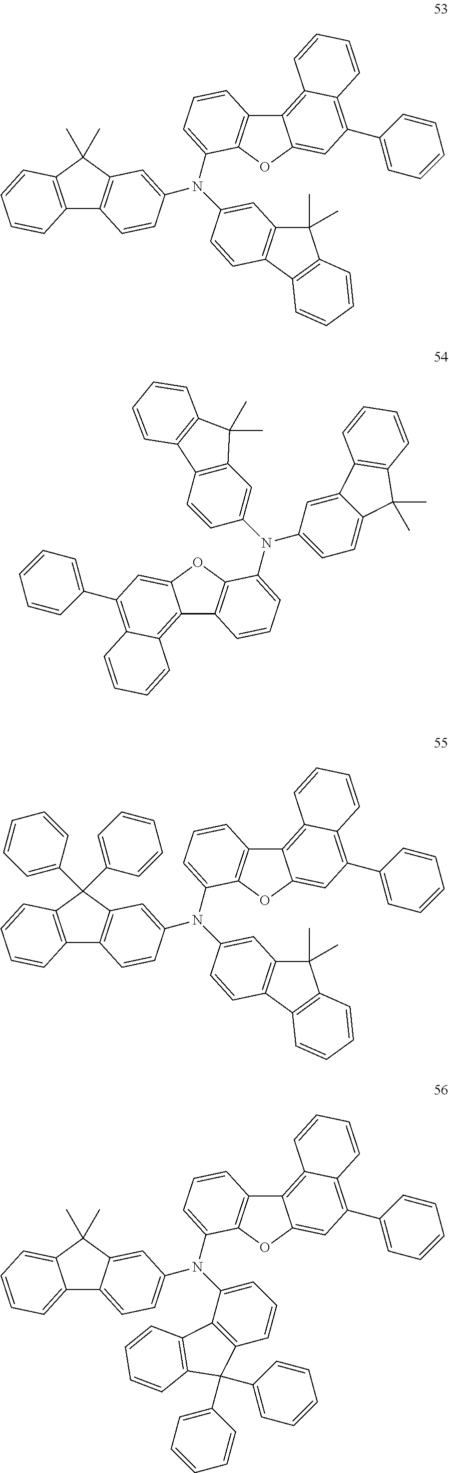 [SMILES: CC1(C)c2ccccc2-c2ccc(N(c3ccc4c(c3)C(C)(C)c3ccccc3-4)c3cccc4c3oc3cc(-c5ccccc5)c5ccccc5c34)cc21 ; CC1(C)c2ccccc2-c2cc(N(c3ccc4c(c3)C(C)(C)c3ccccc3-4)c3cccc4c3oc3cc(-c5ccccc5)c5ccccc5c34)ccc21 ; CC1(C)c2ccccc2-c2ccc(N(c3ccc4c(c3)C(c3ccccc3)(c3ccccc3)c3ccccc3-4)c3cccc4c3oc3cc(-c5ccccc5)c5ccccc5c34)cc21 ; CC1(C)c2ccccc2-c2ccc(N(c3cccc4c3-c3ccccc3C4(c3ccccc3)c3ccccc3)c3cccc4c3oc3cc(-c5ccccc5)c5ccccc5c34)cc21]

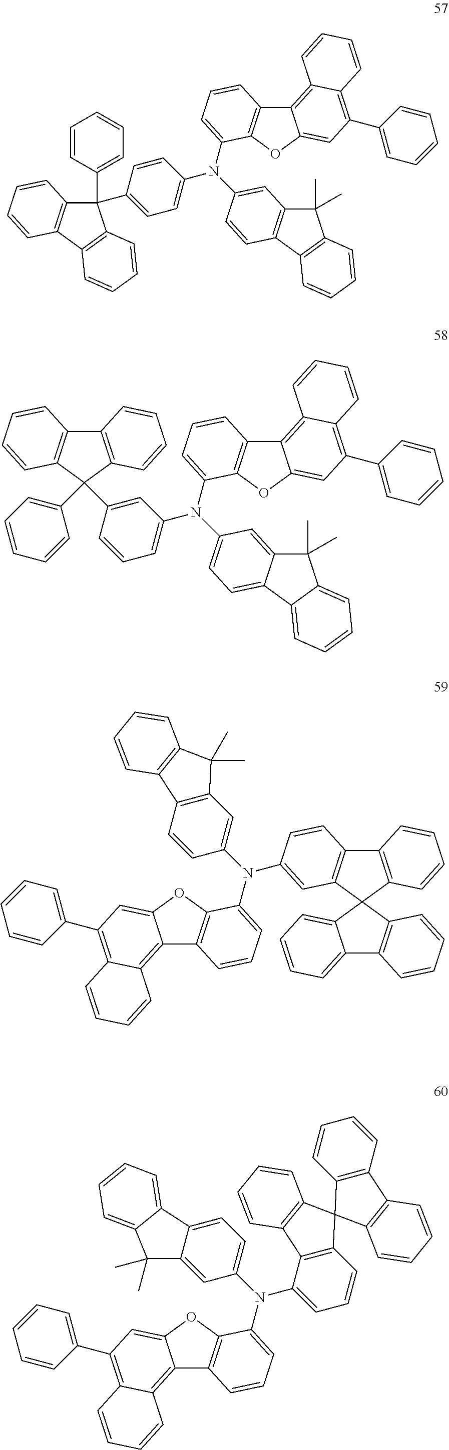 [SMILES: CC1(C)c2ccccc2-c2ccc(N(c3ccc(C4(c5ccccc5)c5ccccc5-c5ccccc54)cc3)c3cccc4c3oc3cc(-c5ccccc5)c5ccccc5c34)cc21 ; CC1(C)c2ccccc2-c2ccc(N(c3cccc(C4(c5ccccc5)c5ccccc5-c5ccccc54)c3)c3cccc4c3oc3cc(-c5ccccc5)c5ccccc5c34)cc21 ; CC1(C)c2ccccc2-c2ccc(N(c3ccc4c(c3)C3(c5ccccc5-c5ccccc53)c3ccccc3-4)c3cccc4c3oc3cc(-c5ccccc5)c5ccccc5c34)cc21 ; CC1(C)c2ccccc2-c2ccc(N(c3cccc4c3-c3ccccc3C43c4ccccc4-c4ccccc43)c3cccc4c3oc3cc(-c5ccccc5)c5ccccc5c34)cc21]

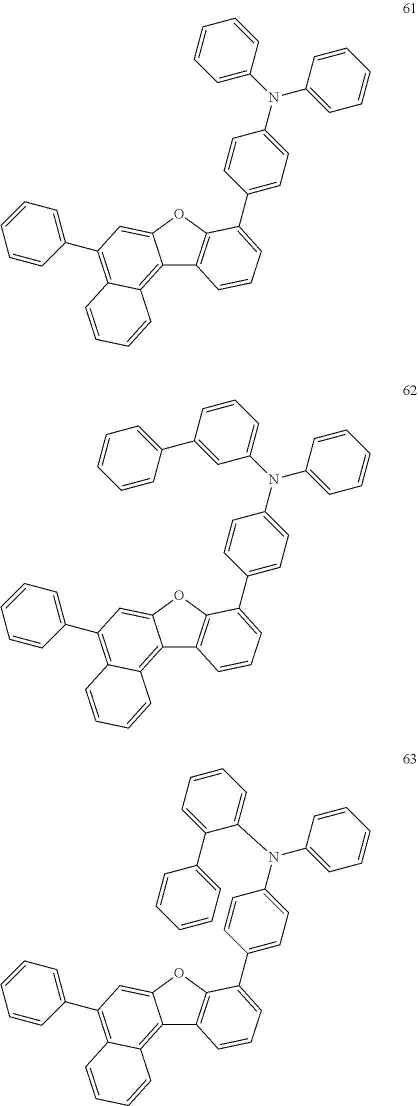 [SMILES: c1ccc(-c2cc3oc4c(-c5ccc(N(c6ccccc6)c6ccccc6)cc5)cccc4c3c3ccccc23)cc1 ; c1ccc(-c2cccc(N(c3ccccc3)c3ccc(-c4cccc5c4oc4cc(-c6ccccc6)c6ccccc6c45)cc3)c2)cc1 ; c1ccc(-c2ccccc2N(c2ccccc2)c2ccc(-c3cccc4c3oc3cc(-c5ccccc5)c5ccccc5c34)cc2)cc1]

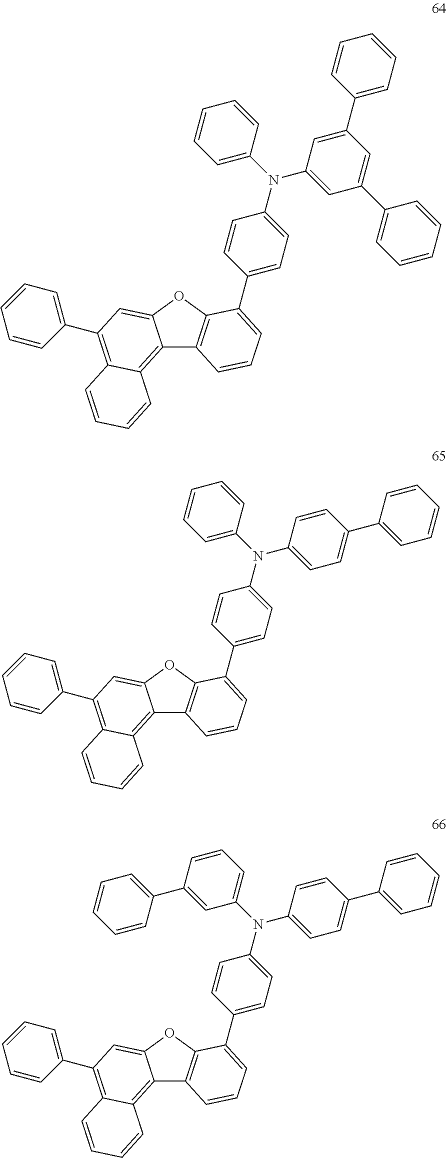 [SMILES: c1ccc(-c2cc(-c3ccccc3)cc(N(c3ccccc3)c3ccc(-c4cccc5c4oc4cc(-c6ccccc6)c6ccccc6c45)cc3)c2)cc1 ; c1ccc(-c2ccc(N(c3ccccc3)c3ccc(-c4cccc5c4oc4cc(-c6ccccc6)c6ccccc6c45)cc3)cc2)cc1 ; c1ccc(-c2ccc(N(c3ccc(-c4cccc5c4oc4cc(-c6ccccc6)c6ccccc6c45)cc3)c3cccc(-c4ccccc4)c3)cc2)cc1]

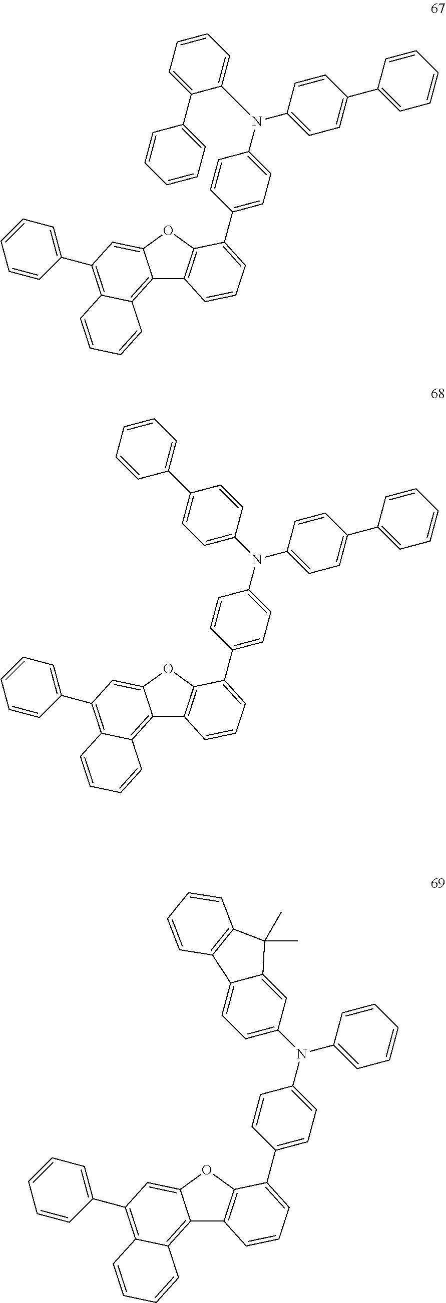 [SMILES: c1ccc(-c2ccc(N(c3ccc(-c4cccc5c4oc4cc(-c6ccccc6)c6ccccc6c45)cc3)c3ccccc3-c3ccccc3)cc2)cc1 ; c1ccc(-c2ccc(N(c3ccc(-c4ccccc4)cc3)c3ccc(-c4cccc5c4oc4cc(-c6ccccc6)c6ccccc6c45)cc3)cc2)cc1 ; CC1(C)c2ccccc2-c2ccc(N(c3ccccc3)c3ccc(-c4cccc5c4oc4cc(-c6ccccc6)c6ccccc6c45)cc3)cc21]

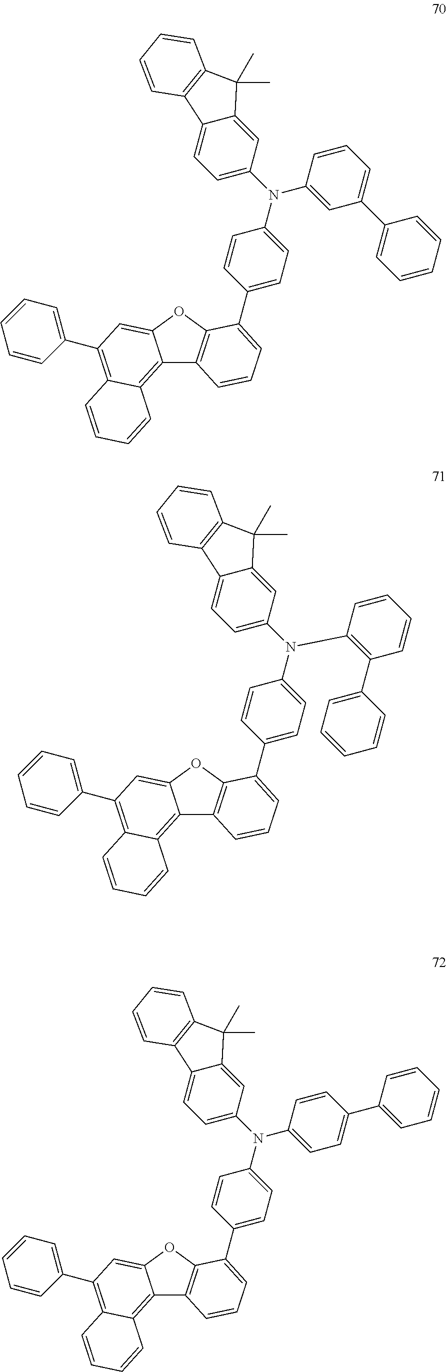 [SMILES: CC1(C)c2ccccc2-c2ccc(N(c3ccc(-c4cccc5c4oc4cc(-c6ccccc6)c6ccccc6c45)cc3)c3cccc(-c4ccccc4)c3)cc21 ; CC1(C)c2ccccc2-c2ccc(N(c3ccc(-c4cccc5c4oc4cc(-c6ccccc6)c6ccccc6c45)cc3)c3ccccc3-c3ccccc3)cc21 ; CC1(C)c2ccccc2-c2ccc(N(c3ccc(-c4ccccc4)cc3)c3ccc(-c4cccc5c4oc4cc(-c6ccccc6)c6ccccc6c45)cc3)cc21]

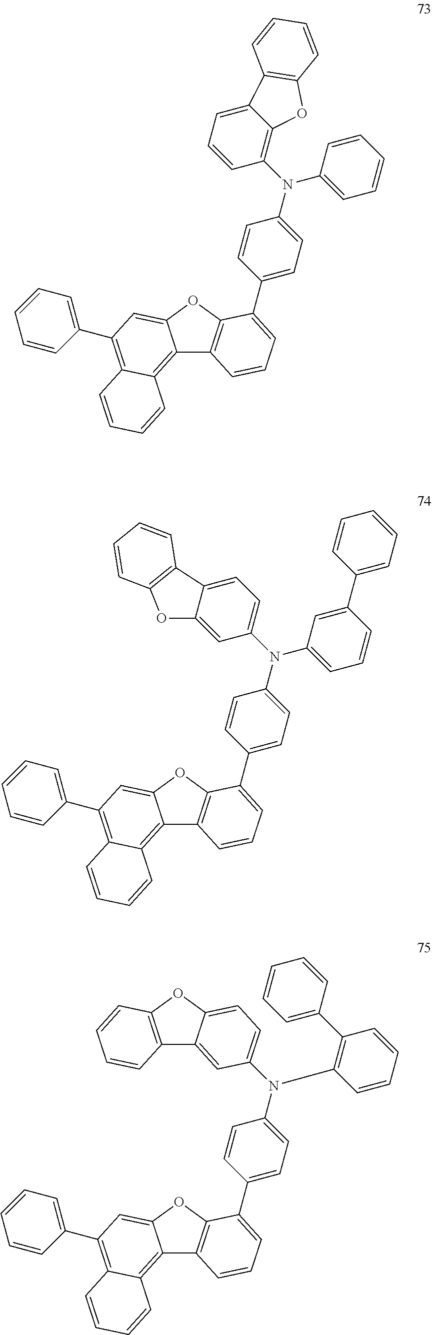 [SMILES: c1ccc(-c2cc3oc4c(-c5ccc(N(c6ccccc6)c6cccc7c6oc6ccccc67)cc5)cccc4c3c3ccccc23)cc1 ; c1ccc(-c2cccc(N(c3ccc(-c4cccc5c4oc4cc(-c6ccccc6)c6ccccc6c45)cc3)c3ccc4c(c3)oc3ccccc34)c2)cc1 ; c1ccc(-c2ccccc2N(c2ccc(-c3cccc4c3oc3cc(-c5ccccc5)c5ccccc5c34)cc2)c2ccc3oc4ccccc4c3c2)cc1]

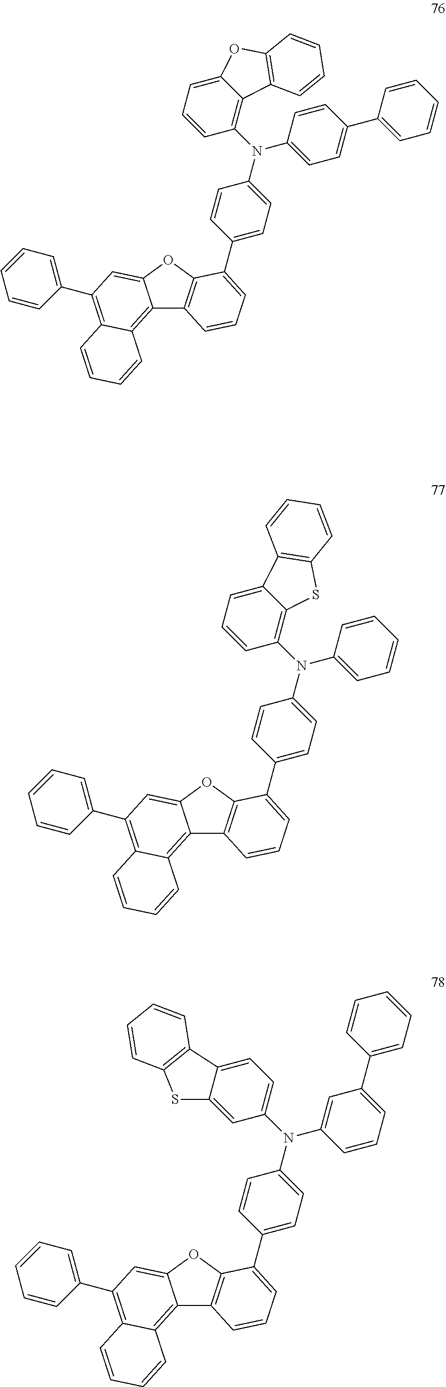 [SMILES: c1ccc(-c2ccc(N(c3ccc(-c4cccc5c4oc4cc(-c6ccccc6)c6ccccc6c45)cc3)c3cccc4oc5ccccc5c34)cc2)cc1 ; c1ccc(-c2cc3oc4c(-c5ccc(N(c6ccccc6)c6cccc7c6sc6ccccc67)cc5)cccc4c3c3ccccc23)cc1 ; c1ccc(-c2cccc(N(c3ccc(-c4cccc5c4oc4cc(-c6ccccc6)c6ccccc6c45)cc3)c3ccc4c(c3)sc3ccccc34)c2)cc1]

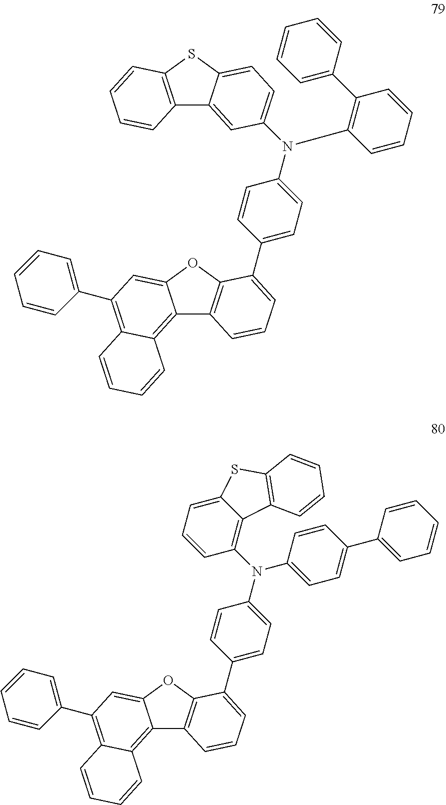 [SMILES: c1ccc(-c2ccccc2N(c2ccc(-c3cccc4c3oc3cc(-c5ccccc5)c5ccccc5c34)cc2)c2ccc3sc4ccccc4c3c2)cc1 ; c1ccc(-c2ccc(N(c3ccc(-c4cccc5c4oc4cc(-c6ccccc6)c6ccccc6c45)cc3)c3cccc4sc5ccccc5c34)cc2)cc1]

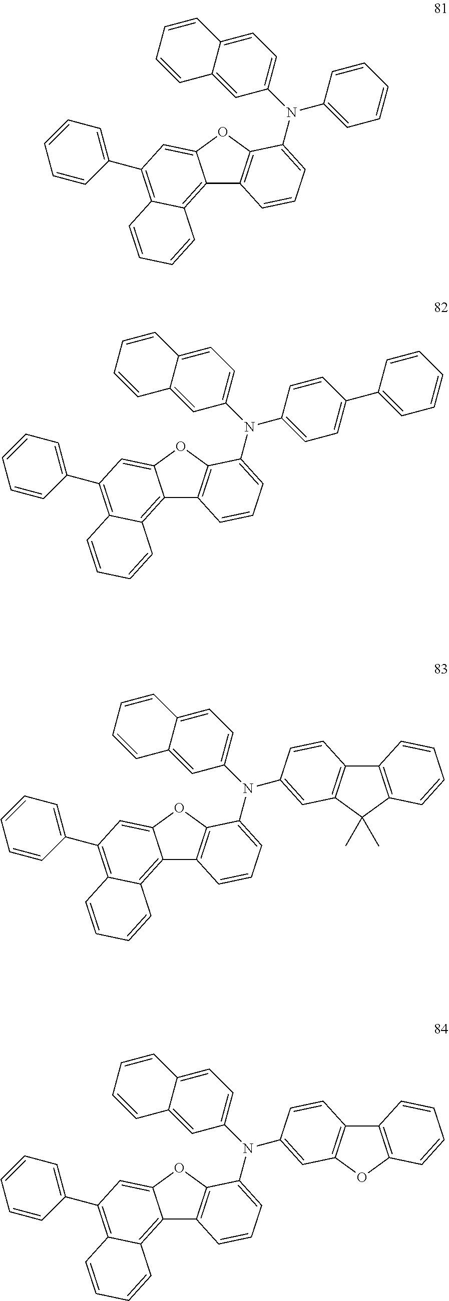 [SMILES: c1ccc(-c2cc3oc4c(N(c5ccccc5)c5ccc6ccccc6c5)cccc4c3c3ccccc23)cc1 ; c1ccc(-c2ccc(N(c3ccc4ccccc4c3)c3cccc4c3oc3cc(-c5ccccc5)c5ccccc5c34)cc2)cc1 ; CC1(C)c2ccccc2-c2ccc(N(c3ccc4ccccc4c3)c3cccc4c3oc3cc(-c5ccccc5)c5ccccc5c34)cc21 ; c1ccc(-c2cc3oc4c(N(c5ccc6ccccc6c5)c5ccc6c(c5)oc5ccccc56)cccc4c3c3ccccc23)cc1]

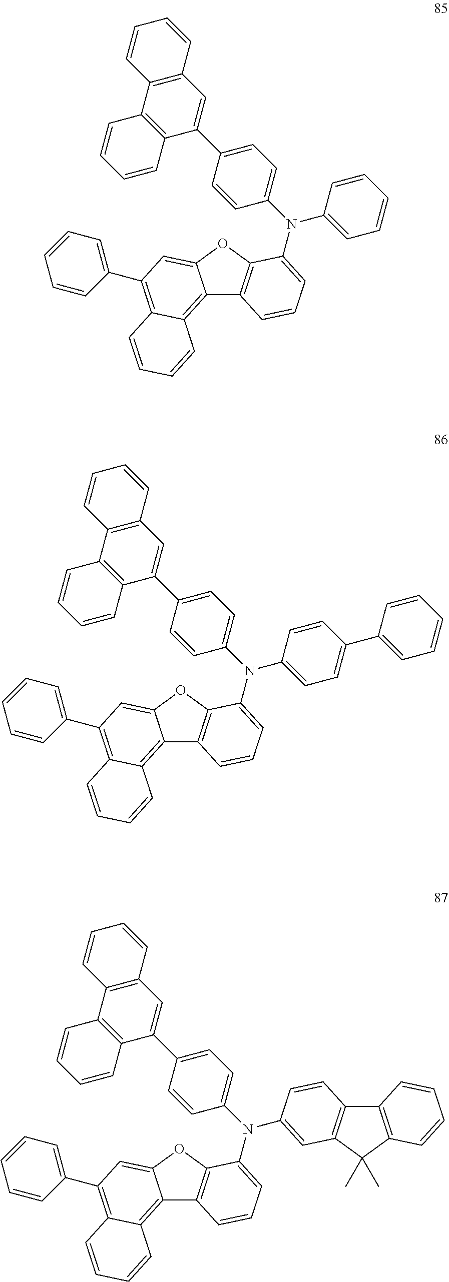 [SMILES: c1ccc(-c2cc3oc4c(N(c5ccccc5)c5ccc(-c6cc7ccccc7c7ccccc67)cc5)cccc4c3c3ccccc23)cc1 ; c1ccc(-c2ccc(N(c3ccc(-c4cc5ccccc5c5ccccc45)cc3)c3cccc4c3oc3cc(-c5ccccc5)c5ccccc5c34)cc2)cc1 ; CC1(C)c2ccccc2-c2ccc(N(c3ccc(-c4cc5ccccc5c5ccccc45)cc3)c3cccc4c3oc3cc(-c5ccccc5)c5ccccc5c34)cc21]

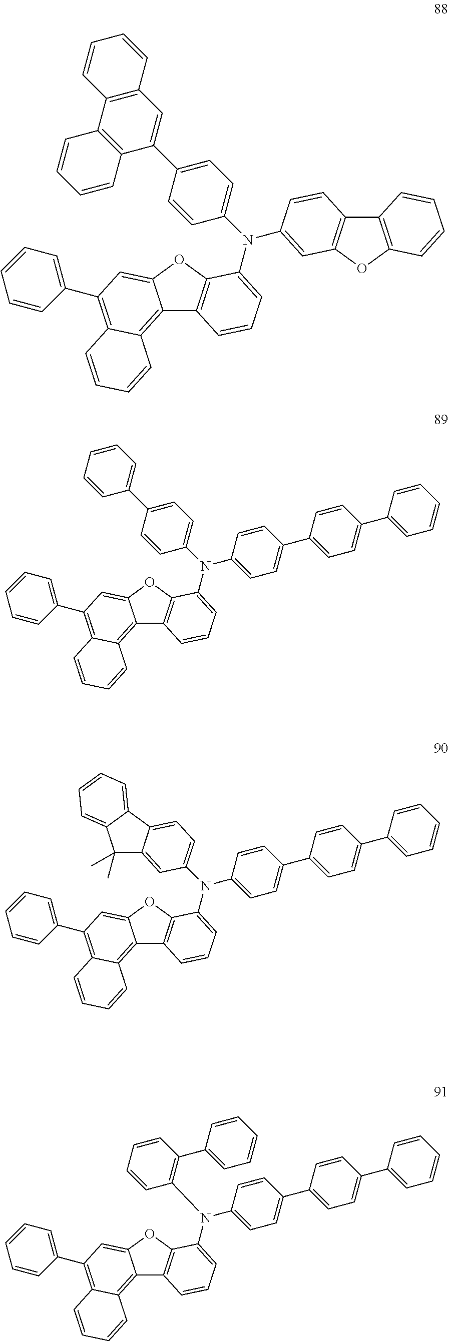 [SMILES: c1ccc(-c2cc3oc4c(N(c5ccc(-c6cc7ccccc7c7ccccc67)cc5)c5ccc6c(c5)oc5ccccc56)cccc4c3c3ccccc23)cc1 ; c1ccc(-c2ccc(-c3ccc(N(c4ccc(-c5ccccc5)cc4)c4cccc5c4oc4cc(-c6ccccc6)c6ccccc6c45)cc3)cc2)cc1 ; CC1(C)c2ccccc2-c2ccc(N(c3ccc(-c4ccc(-c5ccccc5)cc4)cc3)c3cccc4c3oc3cc(-c5ccccc5)c5ccccc5c34)cc21 ; c1ccc(-c2ccc(-c3ccc(N(c4ccccc4-c4ccccc4)c4cccc5c4oc4cc(-c6ccccc6)c6ccccc6c45)cc3)cc2)cc1]

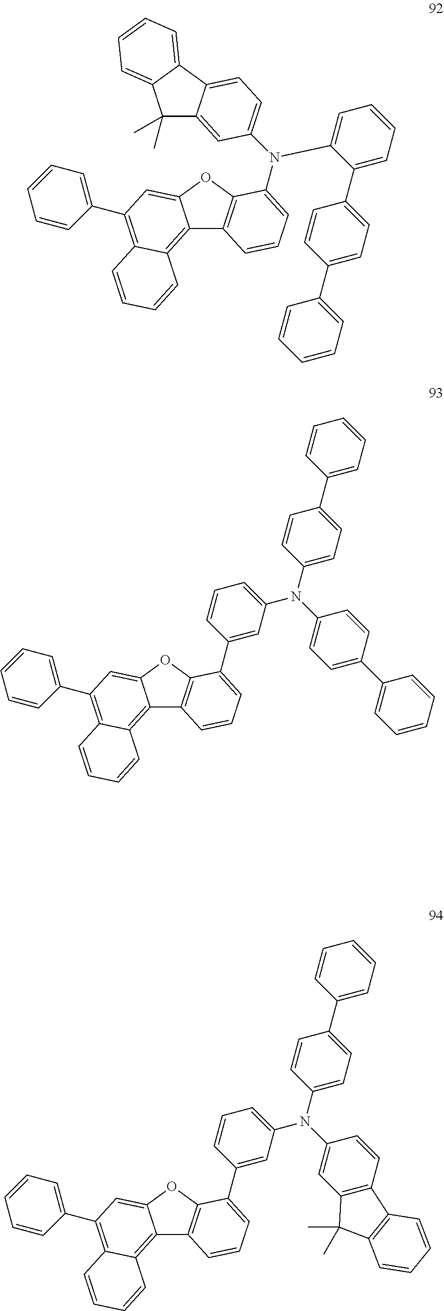 [SMILES: CC1(C)c2ccccc2-c2ccc(N(c3ccccc3-c3ccc(-c4ccccc4)cc3)c3cccc4c3oc3cc(-c5ccccc5)c5ccccc5c34)cc21 ; c1ccc(-c2ccc(N(c3ccc(-c4ccccc4)cc3)c3cccc(-c4cccc5c4oc4cc(-c6ccccc6)c6ccccc6c45)c3)cc2)cc1 ; CC1(C)c2ccccc2-c2ccc(N(c3ccc(-c4ccccc4)cc3)c3cccc(-c4cccc5c4oc4cc(-c6ccccc6)c6ccccc6c45)c3)cc21]

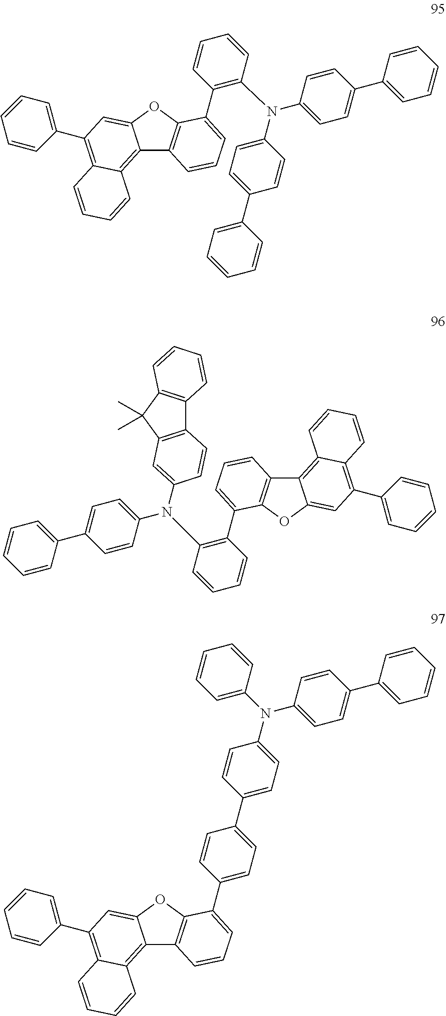 [SMILES: c1ccc(-c2ccc(N(c3ccc(-c4ccccc4)cc3)c3ccccc3-c3cccc4c3oc3cc(-c5ccccc5)c5ccccc5c34)cc2)cc1 ; CC1(C)c2ccccc2-c2ccc(N(c3ccc(-c4ccccc4)cc3)c3ccccc3-c3cccc4c3oc3cc(-c5ccccc5)c5ccccc5c34)cc21 ; c1ccc(-c2ccc(N(c3ccccc3)c3ccc(-c4ccc(-c5cccc6c5oc5cc(-c7ccccc7)c7ccccc7c56)cc4)cc3)cc2)cc1]

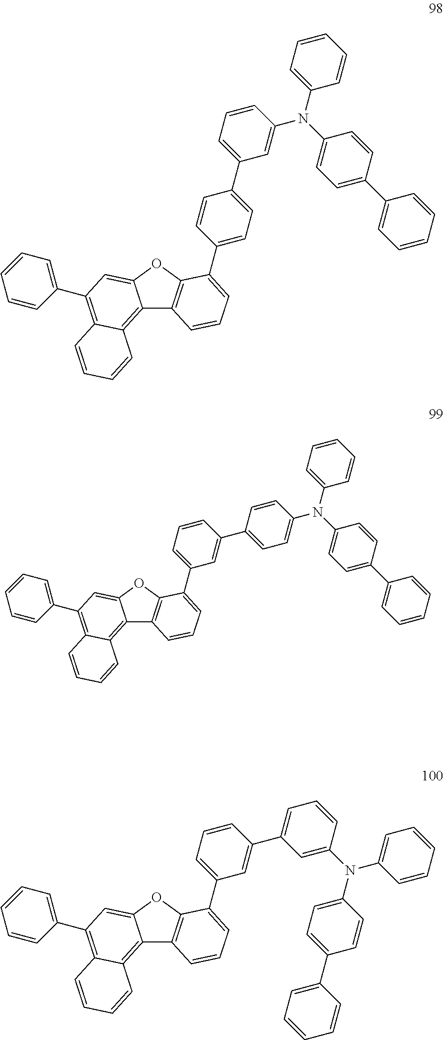 [SMILES: c1ccc(-c2ccc(N(c3ccccc3)c3cccc(-c4ccc(-c5cccc6c5oc5cc(-c7ccccc7)c7ccccc7c56)cc4)c3)cc2)cc1 ; c1ccc(-c2ccc(N(c3ccccc3)c3ccc(-c4cccc(-c5cccc6c5oc5cc(-c7ccccc7)c7ccccc7c56)c4)cc3)cc2)cc1 ; c1ccc(-c2ccc(N(c3ccccc3)c3cccc(-c4cccc(-c5cccc6c5oc5cc(-c7ccccc7)c7ccccc7c56)c4)c3)cc2)cc1]

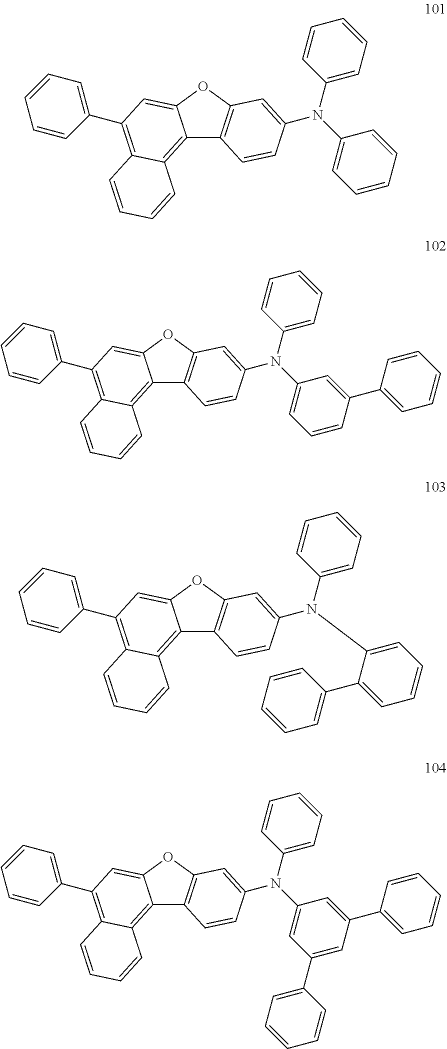 [SMILES: c1ccc(-c2cc3oc4cc(N(c5ccccc5)c5ccccc5)ccc4c3c3ccccc23)cc1 ; c1ccc(-c2cccc(N(c3ccccc3)c3ccc4c(c3)oc3cc(-c5ccccc5)c5ccccc5c34)c2)cc1 ; c1ccc(-c2ccccc2N(c2ccccc2)c2ccc3c(c2)oc2cc(-c4ccccc4)c4ccccc4c23)cc1 ; c1ccc(-c2cc(-c3ccccc3)cc(N(c3ccccc3)c3ccc4c(c3)oc3cc(-c5ccccc5)c5ccccc5c34)c2)cc1]

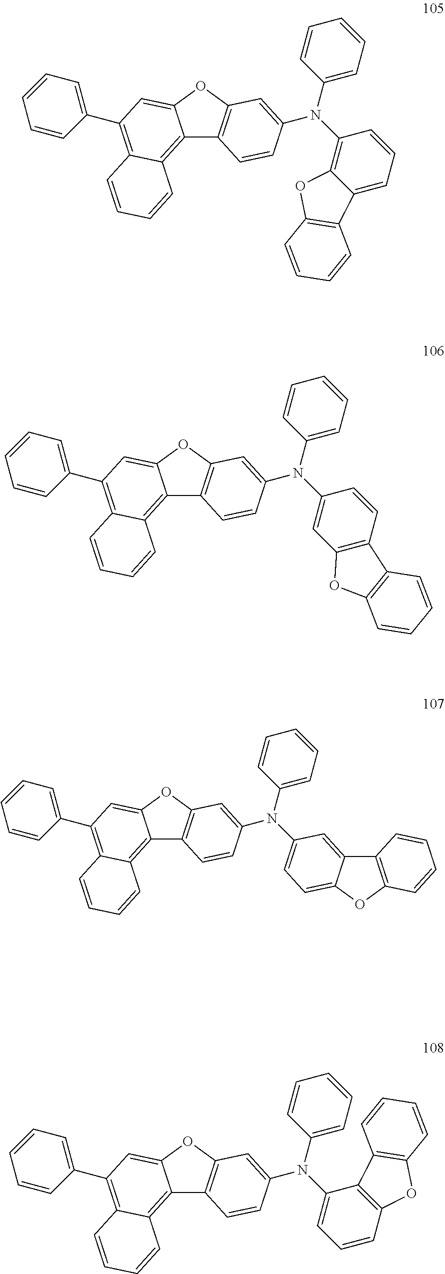 [SMILES: c1ccc(-c2cc3oc4cc(N(c5ccccc5)c5cccc6c5oc5ccccc56)ccc4c3c3ccccc23)cc1 ; c1ccc(-c2cc3oc4cc(N(c5ccccc5)c5ccc6c(c5)oc5ccccc56)ccc4c3c3ccccc23)cc1 ; c1ccc(-c2cc3oc4cc(N(c5ccccc5)c5ccc6oc7ccccc7c6c5)ccc4c3c3ccccc23)cc1 ; c1ccc(-c2cc3oc4cc(N(c5ccccc5)c5cccc6oc7ccccc7c56)ccc4c3c3ccccc23)cc1]

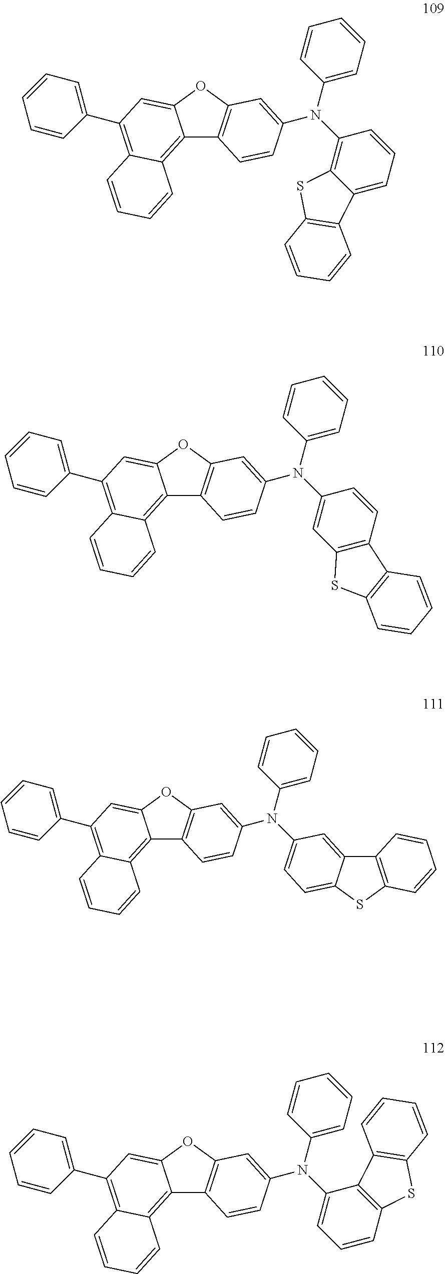 [SMILES: c1ccc(-c2cc3oc4cc(N(c5ccccc5)c5cccc6c5sc5ccccc56)ccc4c3c3ccccc23)cc1 ; c1ccc(-c2cc3oc4cc(N(c5ccccc5)c5ccc6c(c5)sc5ccccc56)ccc4c3c3ccccc23)cc1 ; c1ccc(-c2cc3oc4cc(N(c5ccccc5)c5ccc6sc7ccccc7c6c5)ccc4c3c3ccccc23)cc1 ; c1ccc(-c2cc3oc4cc(N(c5ccccc5)c5cccc6sc7ccccc7c56)ccc4c3c3ccccc23)cc1]

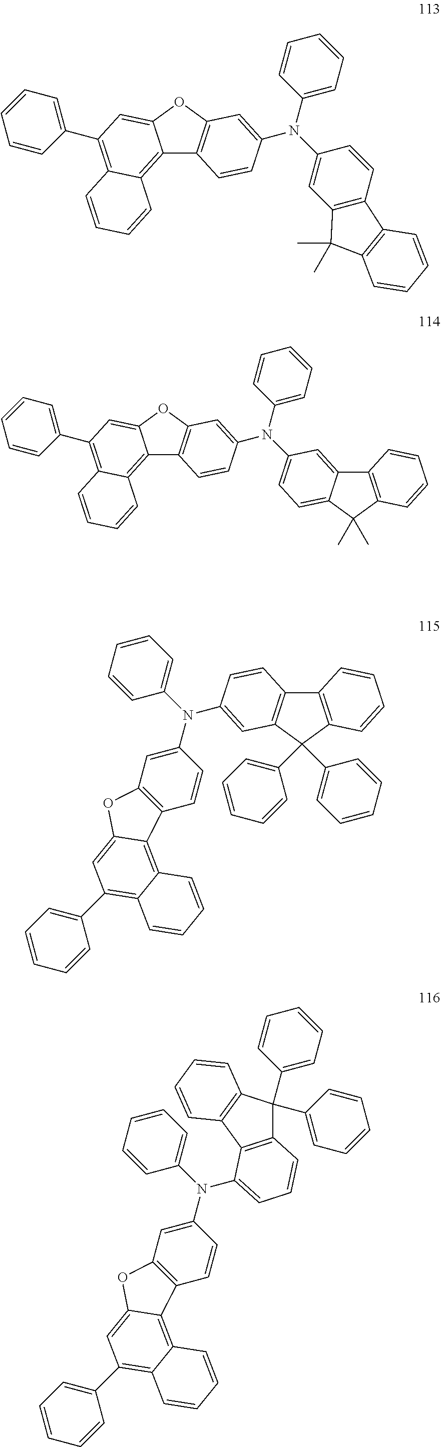 [SMILES: CC1(C)c2ccccc2-c2ccc(N(c3ccccc3)c3ccc4c(c3)oc3cc(-c5ccccc5)c5ccccc5c34)cc21 ; CC1(C)c2ccccc2-c2cc(N(c3ccccc3)c3ccc4c(c3)oc3cc(-c5ccccc5)c5ccccc5c34)ccc21 ; c1ccc(-c2cc3oc4cc(N(c5ccccc5)c5ccc6c(c5)C(c5ccccc5)(c5ccccc5)c5ccccc5-6)ccc4c3c3ccccc23)cc1 ; c1ccc(-c2cc3oc4cc(N(c5ccccc5)c5cccc6c5-c5ccccc5C6(c5ccccc5)c5ccccc5)ccc4c3c3ccccc23)cc1]

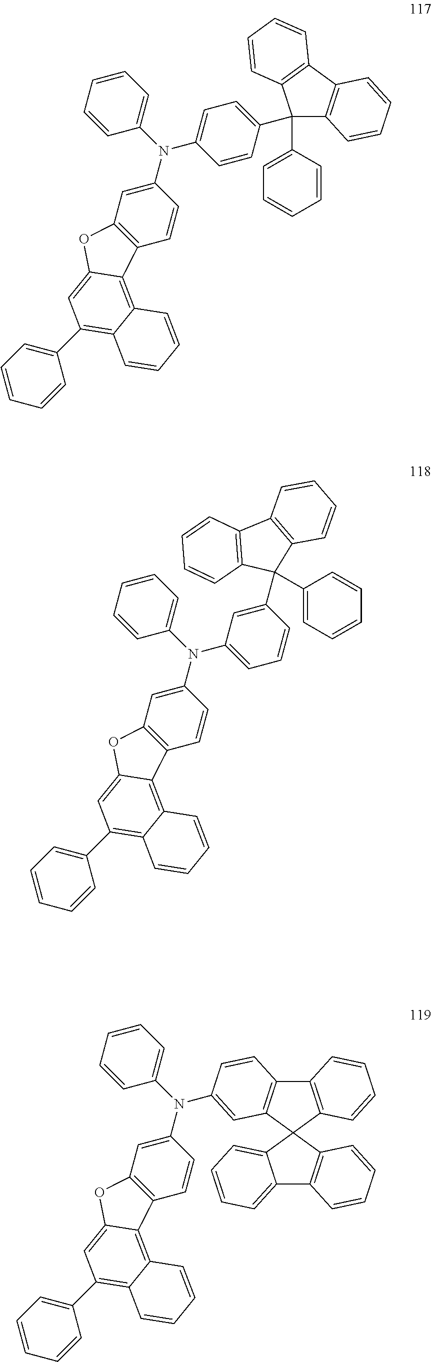 [SMILES: c1ccc(-c2cc3oc4cc(N(c5ccccc5)c5ccc(C6(c7ccccc7)c7ccccc7-c7ccccc76)cc5)ccc4c3c3ccccc23)cc1 ; c1ccc(-c2cc3oc4cc(N(c5ccccc5)c5cccc(C6(c7ccccc7)c7ccccc7-c7ccccc76)c5)ccc4c3c3ccccc23)cc1 ; c1ccc(-c2cc3oc4cc(N(c5ccccc5)c5ccc6c(c5)C5(c7ccccc7-c7ccccc75)c5ccccc5-6)ccc4c3c3ccccc23)cc1]

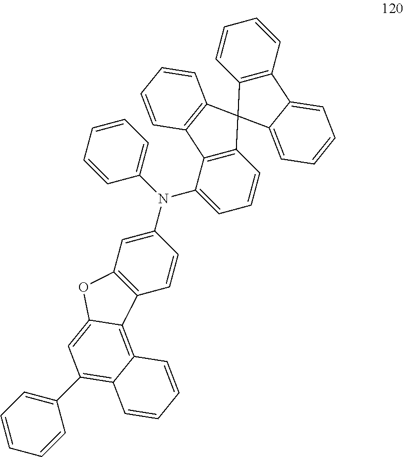 [SMILES: c1ccc(-c2cc3oc4cc(N(c5ccccc5)c5cccc6c5-c5ccccc5C65c6ccccc6-c6ccccc65)ccc4c3c3ccccc23)cc1]

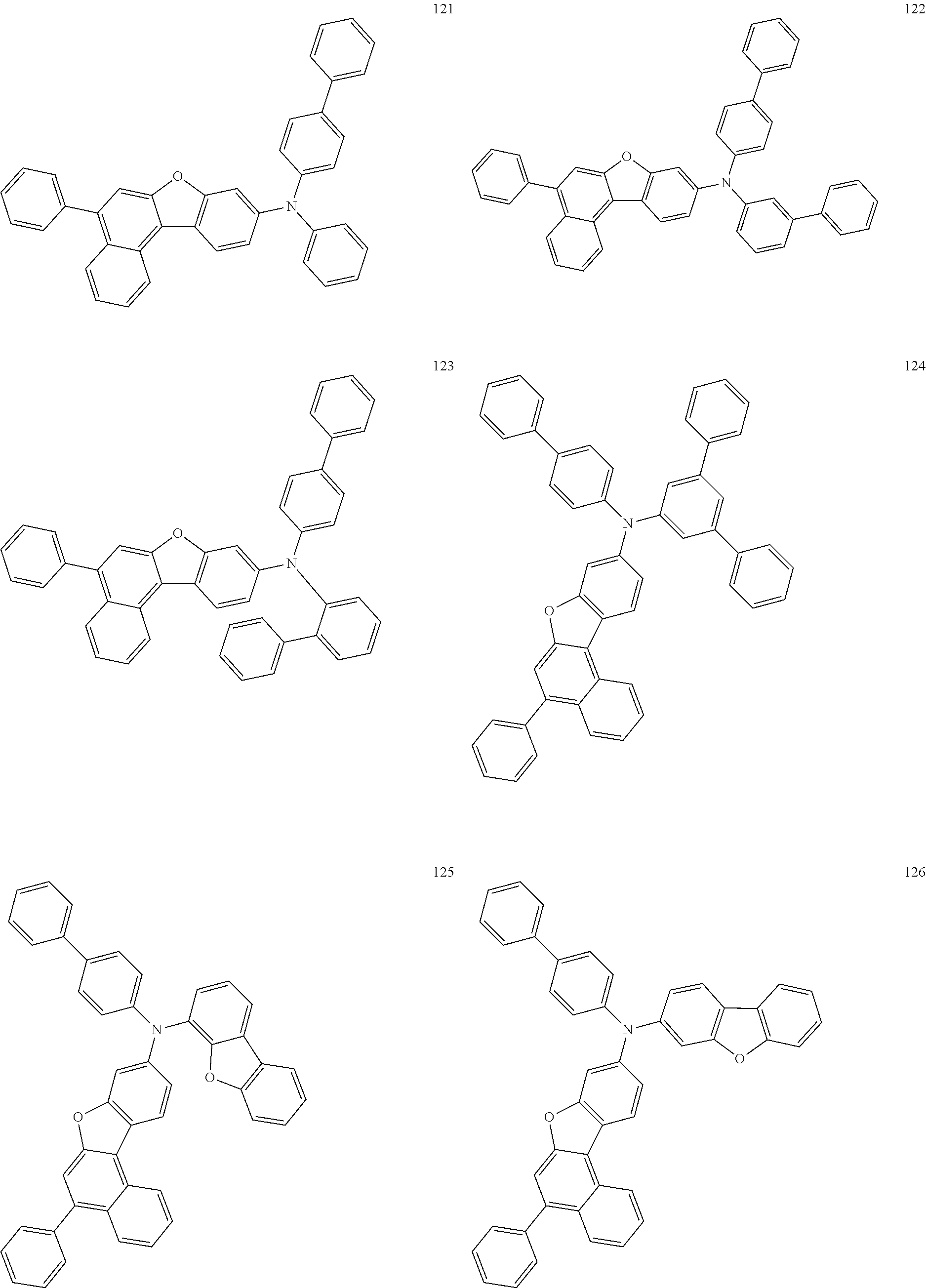

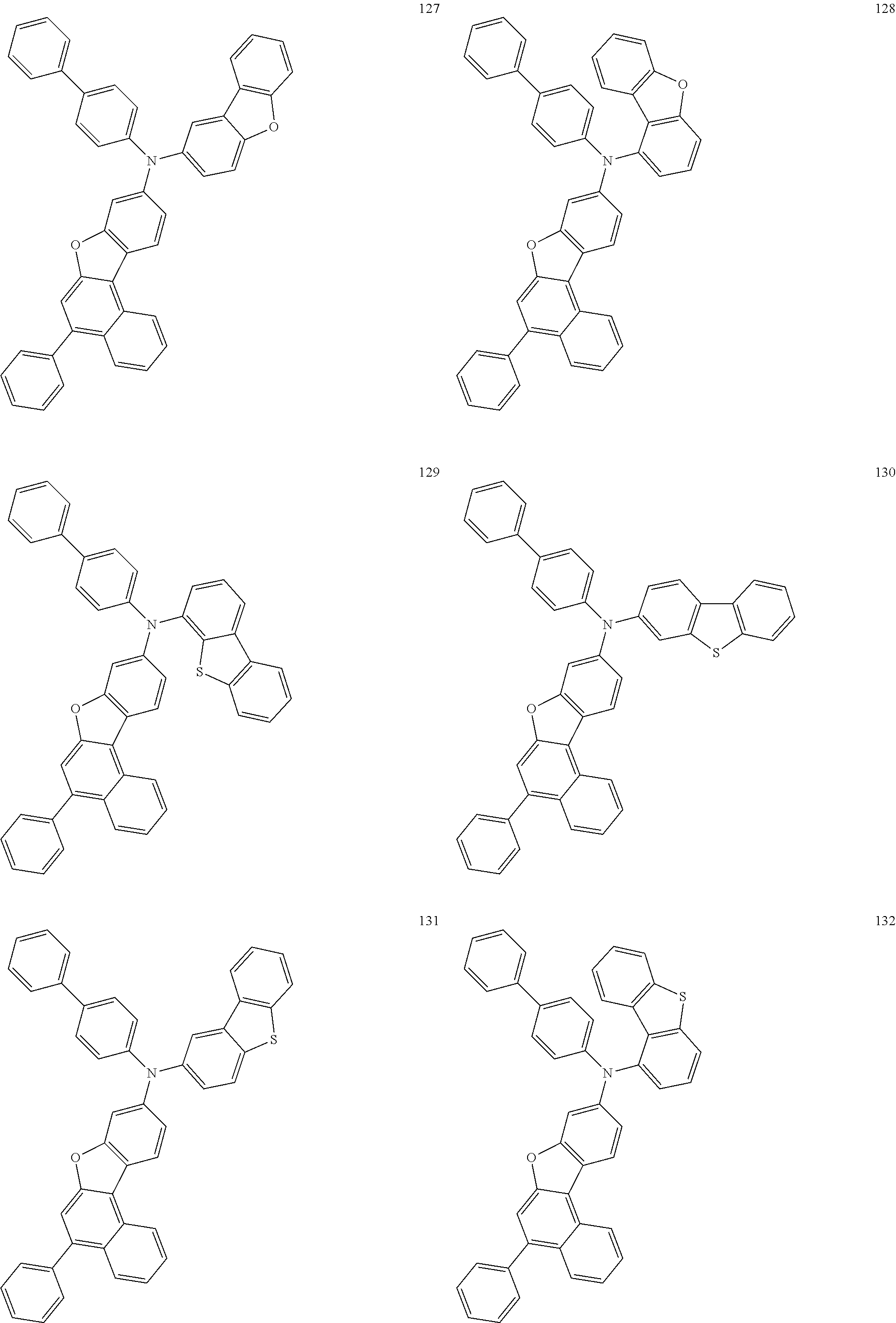

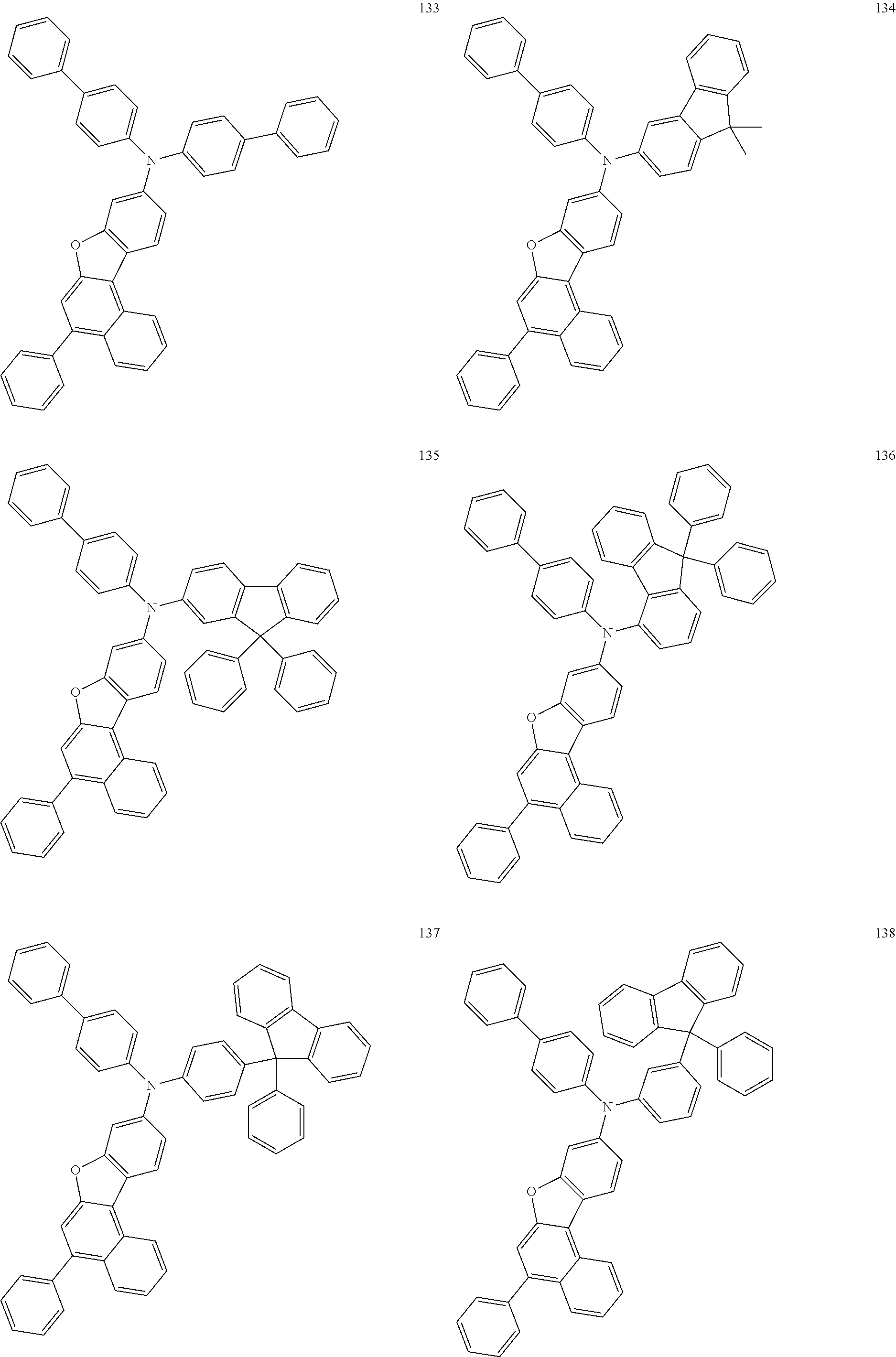

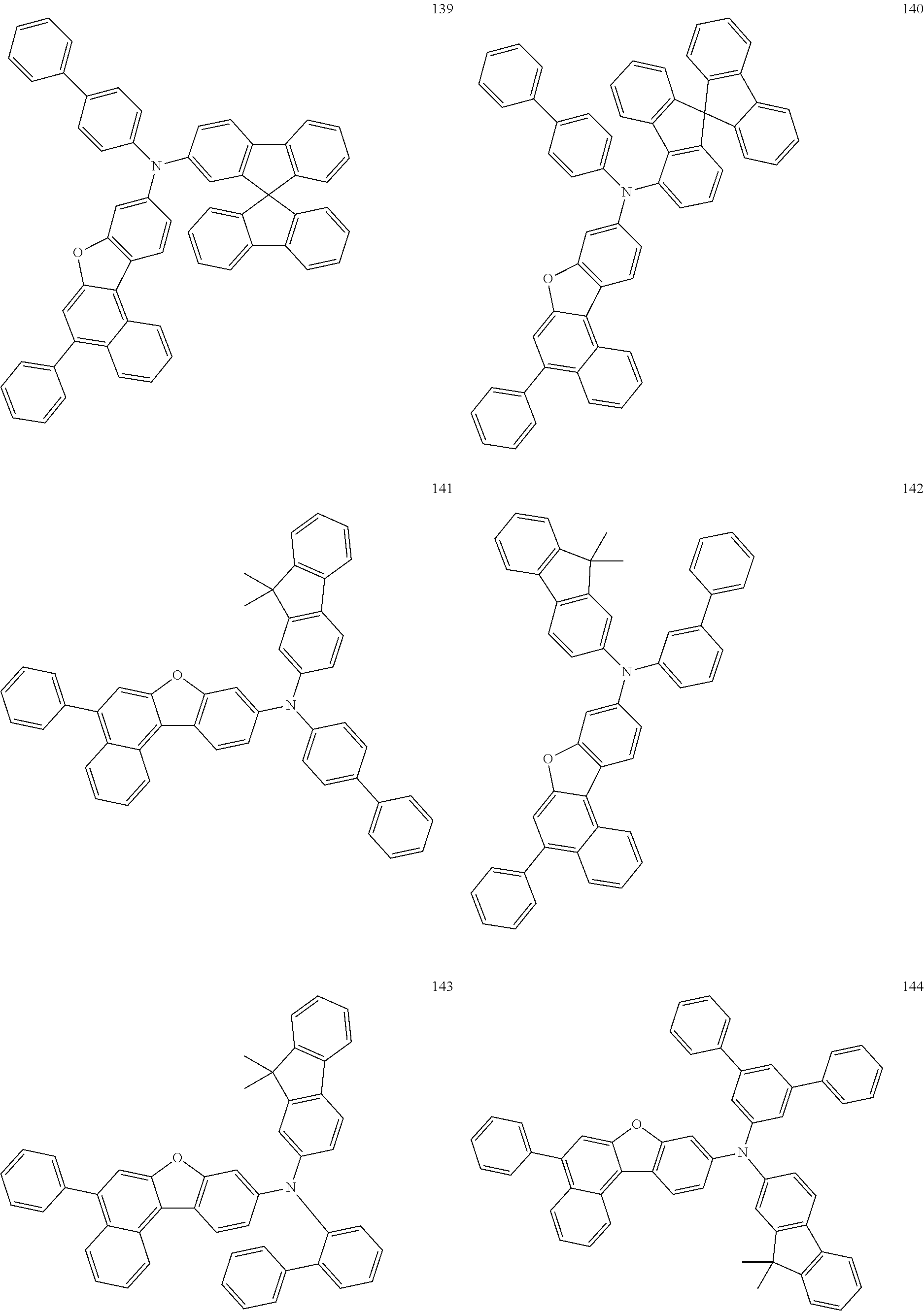

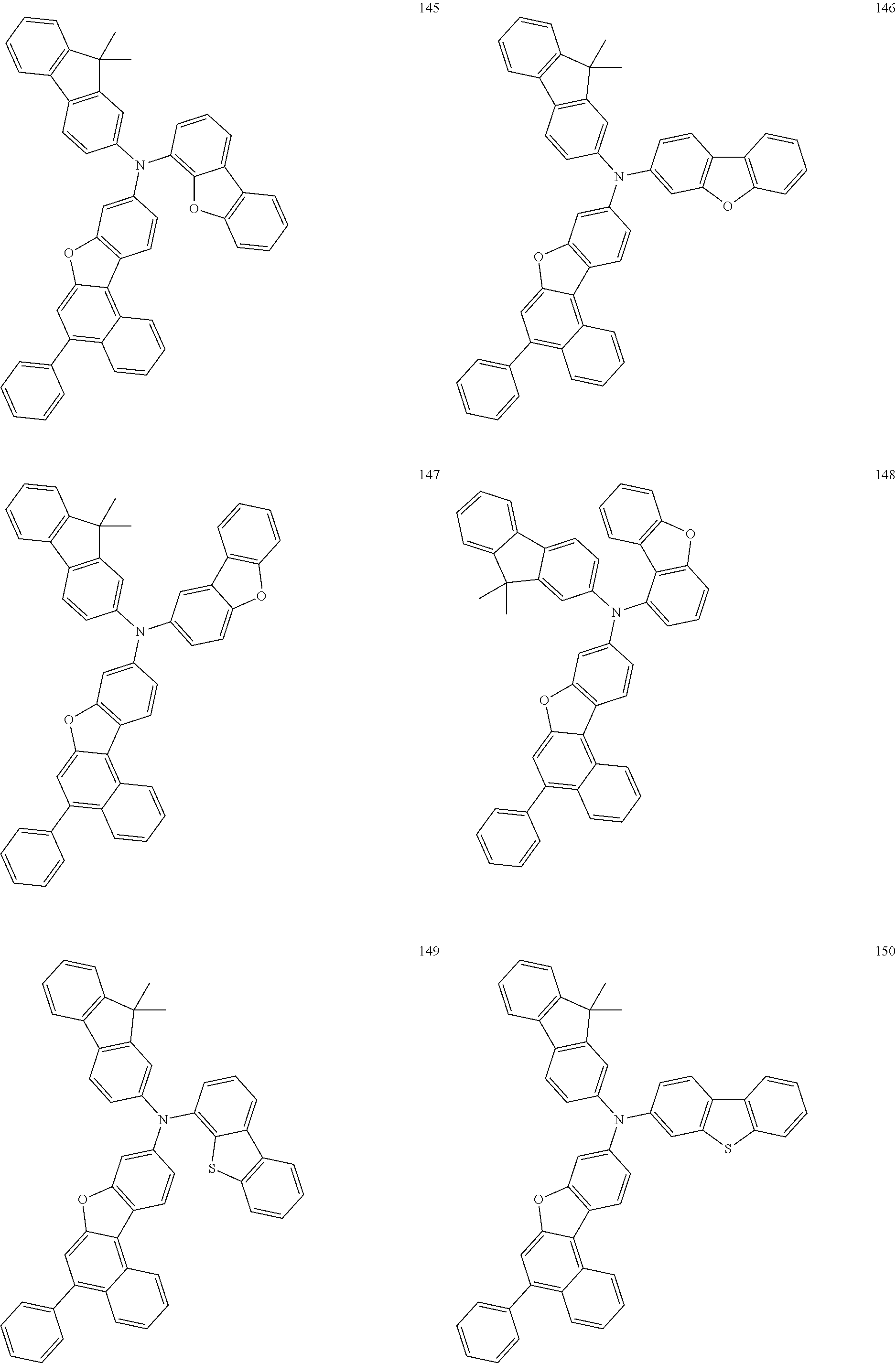

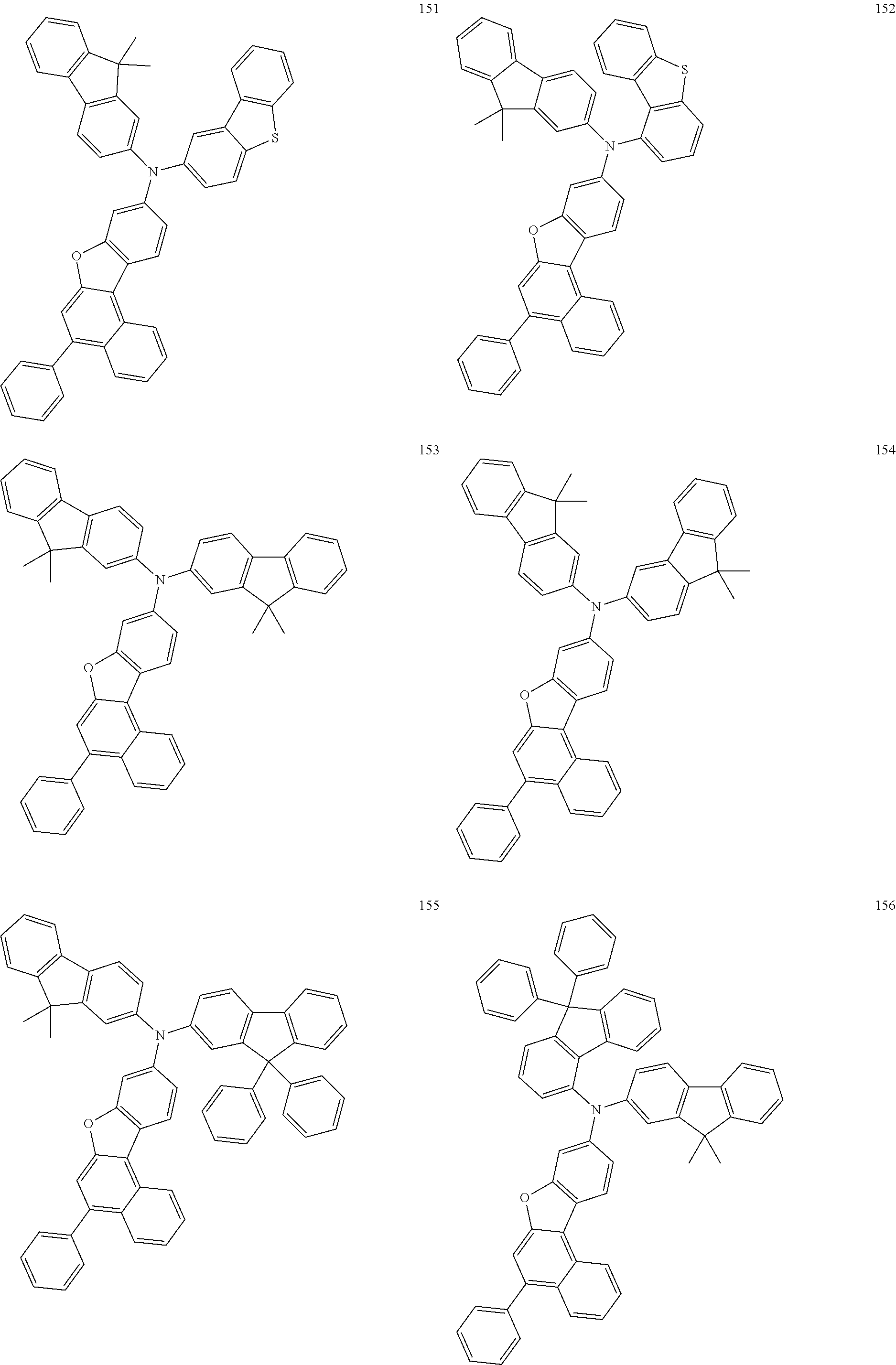

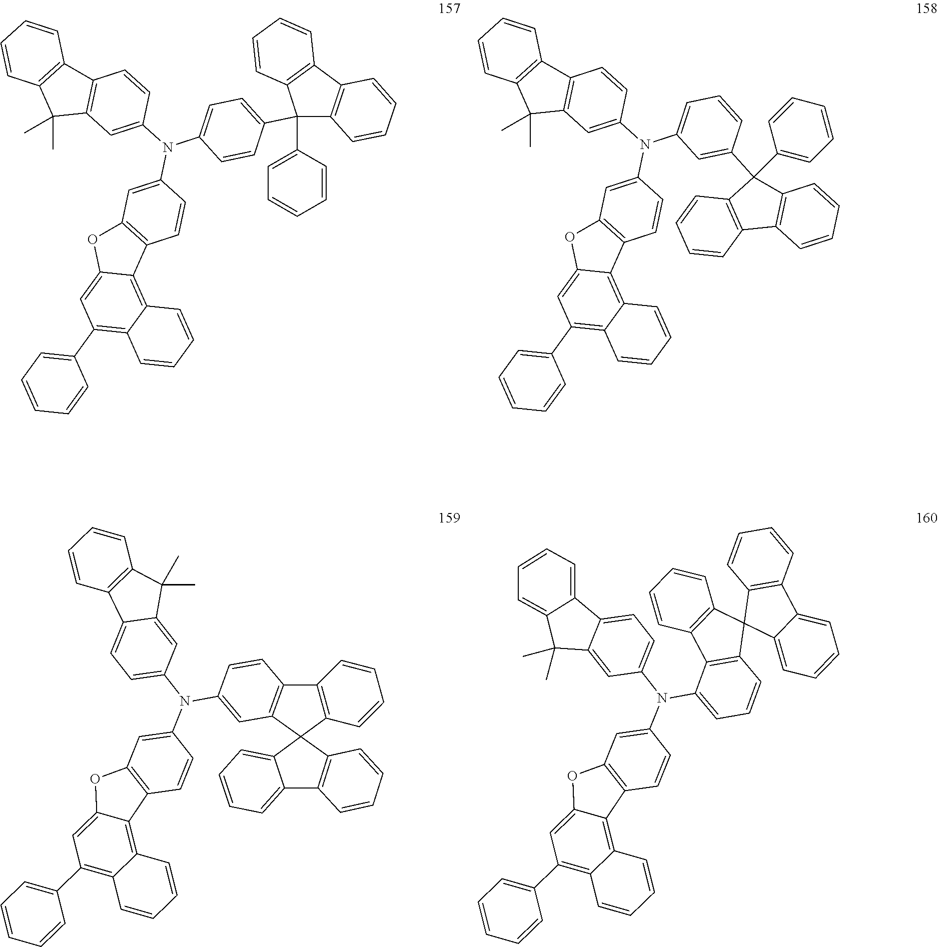 [SMILES: CC1(C)c2ccccc2-c2ccc(N(c3ccc(C4(c5ccccc5)c5ccccc5-c5ccccc54)cc3)c3ccc4c(c3)oc3cc(-c5ccccc5)c5ccccc5c34)cc21 ; CC1(C)c2ccccc2-c2ccc(N(c3cccc(C4(c5ccccc5)c5ccccc5-c5ccccc54)c3)c3ccc4c(c3)oc3cc(-c5ccccc5)c5ccccc5c34)cc21 ; CC1(C)c2ccccc2-c2ccc(N(c3ccc4c(c3)C3(c5ccccc5-c5ccccc53)c3ccccc3-4)c3ccc4c(c3)oc3cc(-c5ccccc5)c5ccccc5c34)cc21 ; CC1(C)c2ccccc2-c2ccc(N(c3ccc4c(c3)oc3cc(-c5ccccc5)c5ccccc5c34)c3cccc4c3-c3ccccc3C43c4ccccc4-c4ccccc43)cc21]

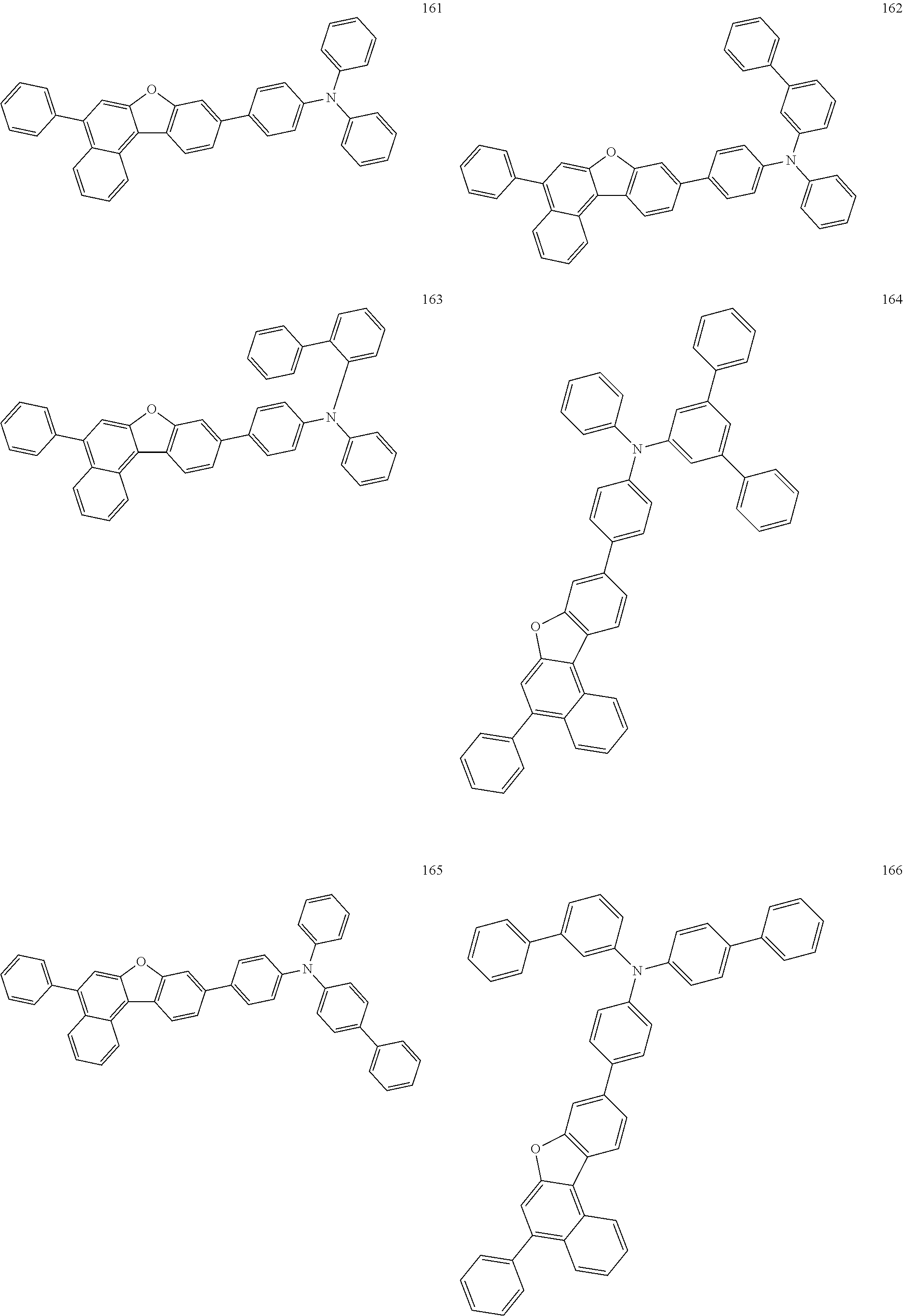

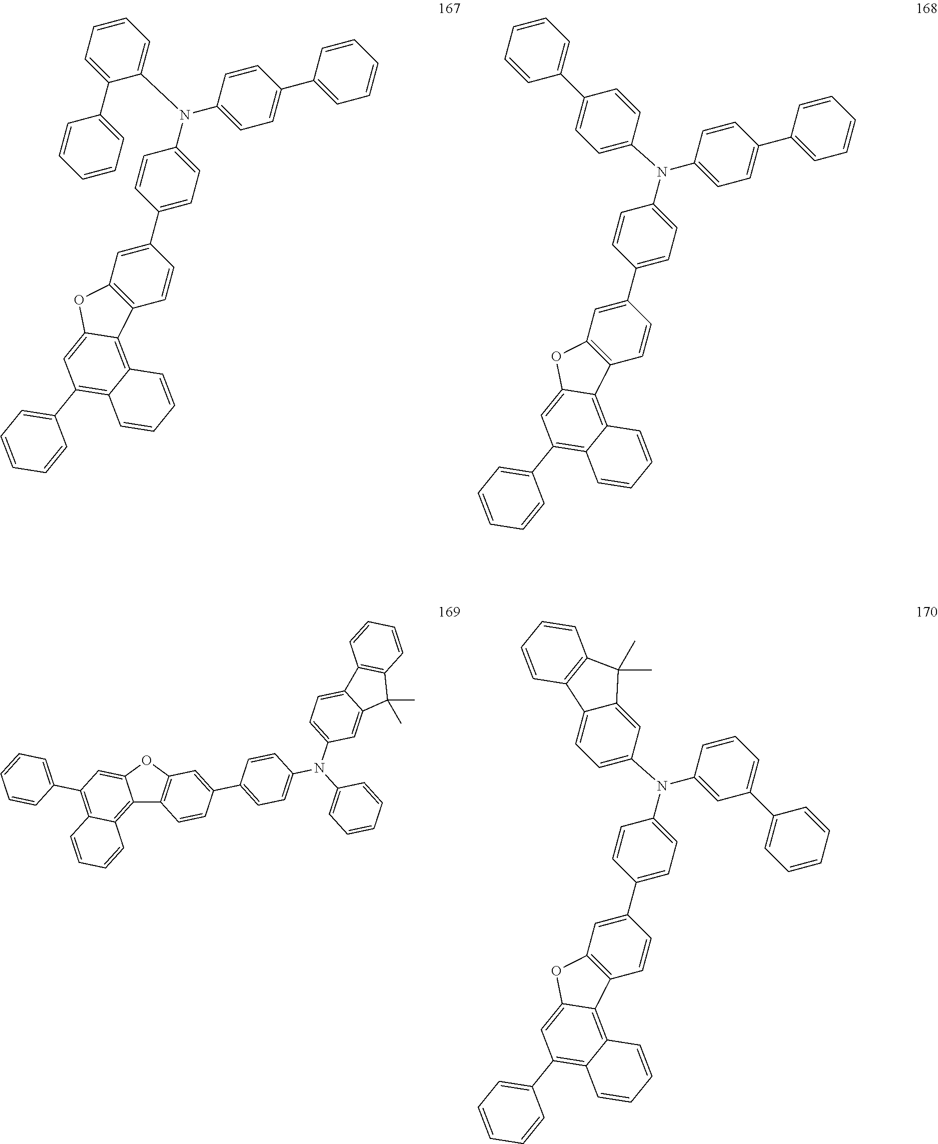 [SMILES: c1ccc(-c2ccc(N(c3ccc(-c4ccc5c(c4)oc4cc(-c6ccccc6)c6ccccc6c45)cc3)c3ccccc3-c3ccccc3)cc2)cc1 ; c1ccc(-c2ccc(N(c3ccc(-c4ccccc4)cc3)c3ccc(-c4ccc5c(c4)oc4cc(-c6ccccc6)c6ccccc6c45)cc3)cc2)cc1 ; CC1(C)c2ccccc2-c2ccc(N(c3ccccc3)c3ccc(-c4ccc5c(c4)oc4cc(-c6ccccc6)c6ccccc6c45)cc3)cc21 ; CC1(C)c2ccccc2-c2ccc(N(c3ccc(-c4ccc5c(c4)oc4cc(-c6ccccc6)c6ccccc6c45)cc3)c3cccc(-c4ccccc4)c3)cc21]

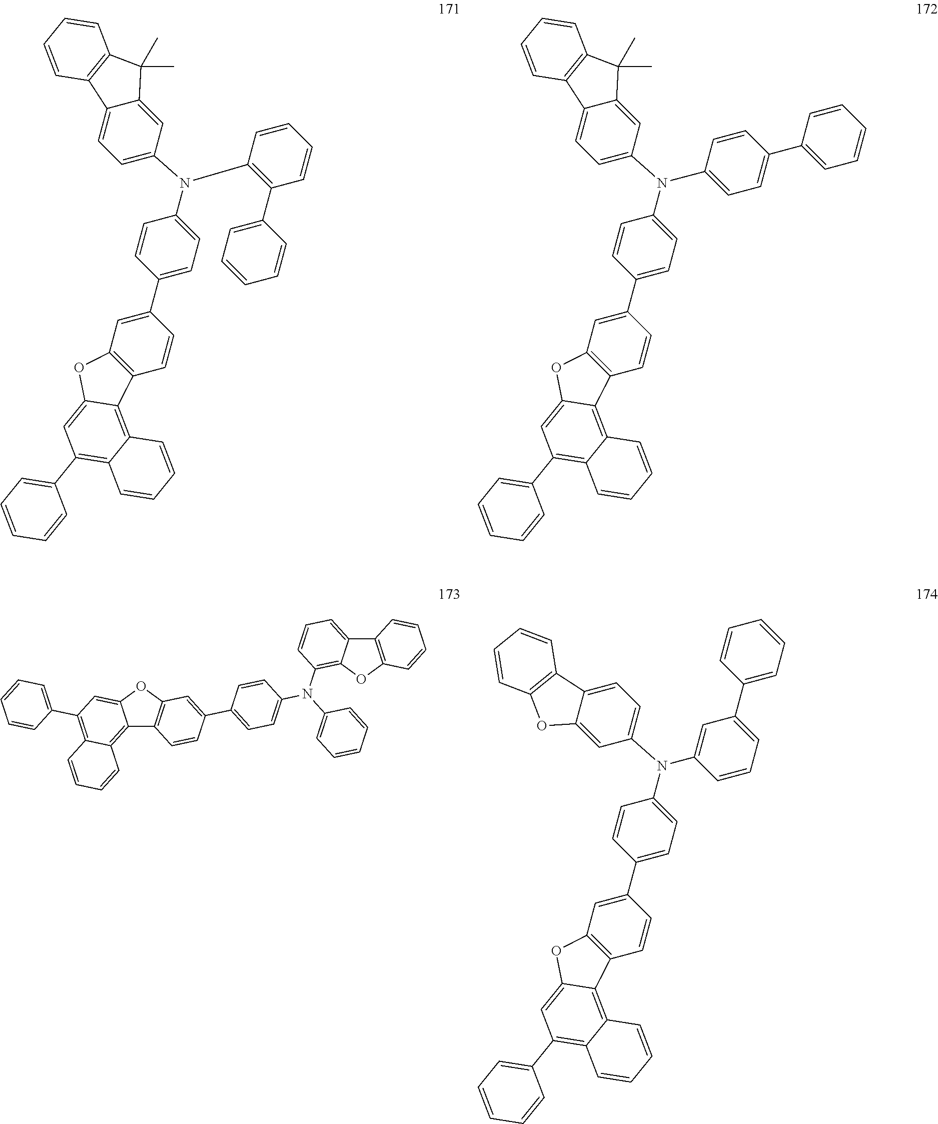 [SMILES: CC1(C)c2ccccc2-c2ccc(N(c3ccc(-c4ccc5c(c4)oc4cc(-c6ccccc6)c6ccccc6c45)cc3)c3ccccc3-c3ccccc3)cc21 ; CC1(C)c2ccccc2-c2ccc(N(c3ccc(-c4ccccc4)cc3)c3ccc(-c4ccc5c(c4)oc4cc(-c6ccccc6)c6ccccc6c45)cc3)cc21 ; c1ccc(-c2cc3oc4cc(-c5ccc(N(c6ccccc6)c6cccc7c6oc6ccccc67)cc5)ccc4c3c3ccccc23)cc1 ; c1ccc(-c2cccc(N(c3ccc(-c4ccc5c(c4)oc4cc(-c6ccccc6)c6ccccc6c45)cc3)c3ccc4c(c3)oc3ccccc34)c2)cc1]

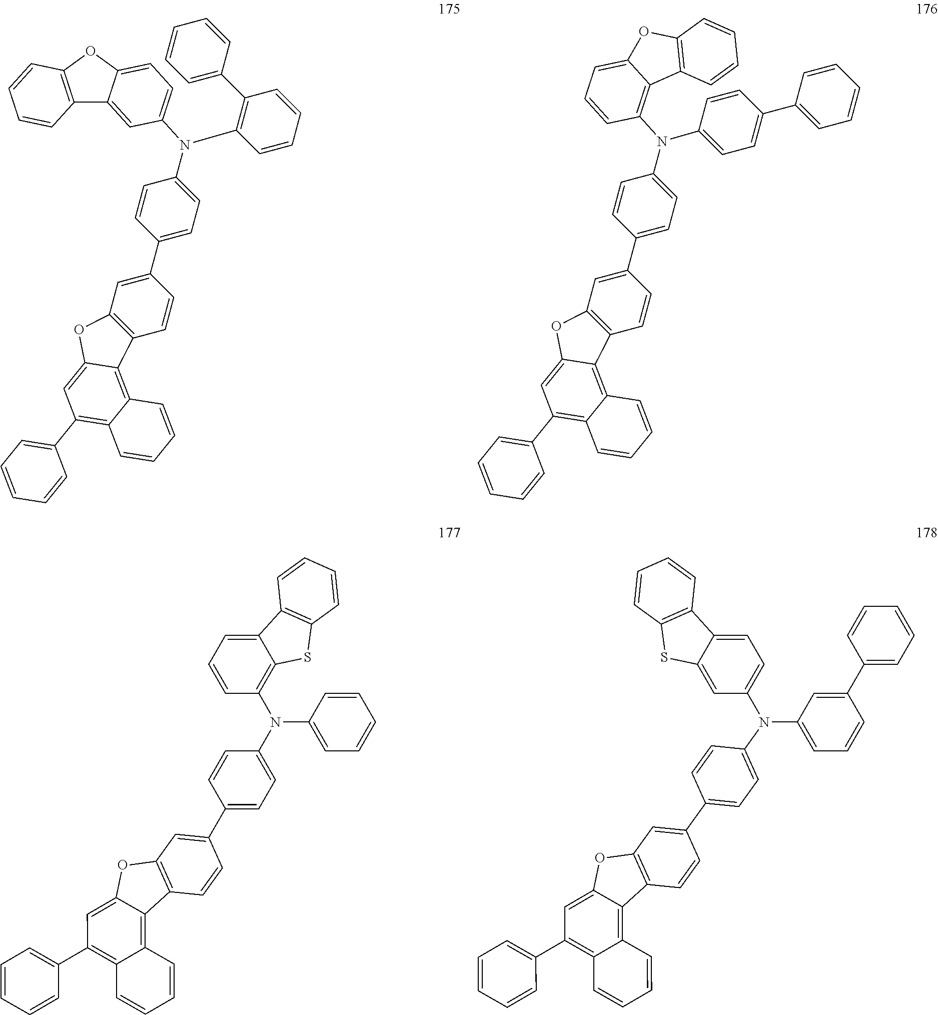 [SMILES: c1ccc(-c2ccccc2N(c2ccc(-c3ccc4c(c3)oc3cc(-c5ccccc5)c5ccccc5c34)cc2)c2ccc3oc4ccccc4c3c2)cc1 ; c1ccc(-c2ccc(N(c3ccc(-c4ccc5c(c4)oc4cc(-c6ccccc6)c6ccccc6c45)cc3)c3cccc4oc5ccccc5c34)cc2)cc1 ; c1ccc(-c2cc3oc4cc(-c5ccc(N(c6ccccc6)c6cccc7c6sc6ccccc67)cc5)ccc4c3c3ccccc23)cc1 ; c1ccc(-c2cccc(N(c3ccc(-c4ccc5c(c4)oc4cc(-c6ccccc6)c6ccccc6c45)cc3)c3ccc4c(c3)sc3ccccc34)c2)cc1]

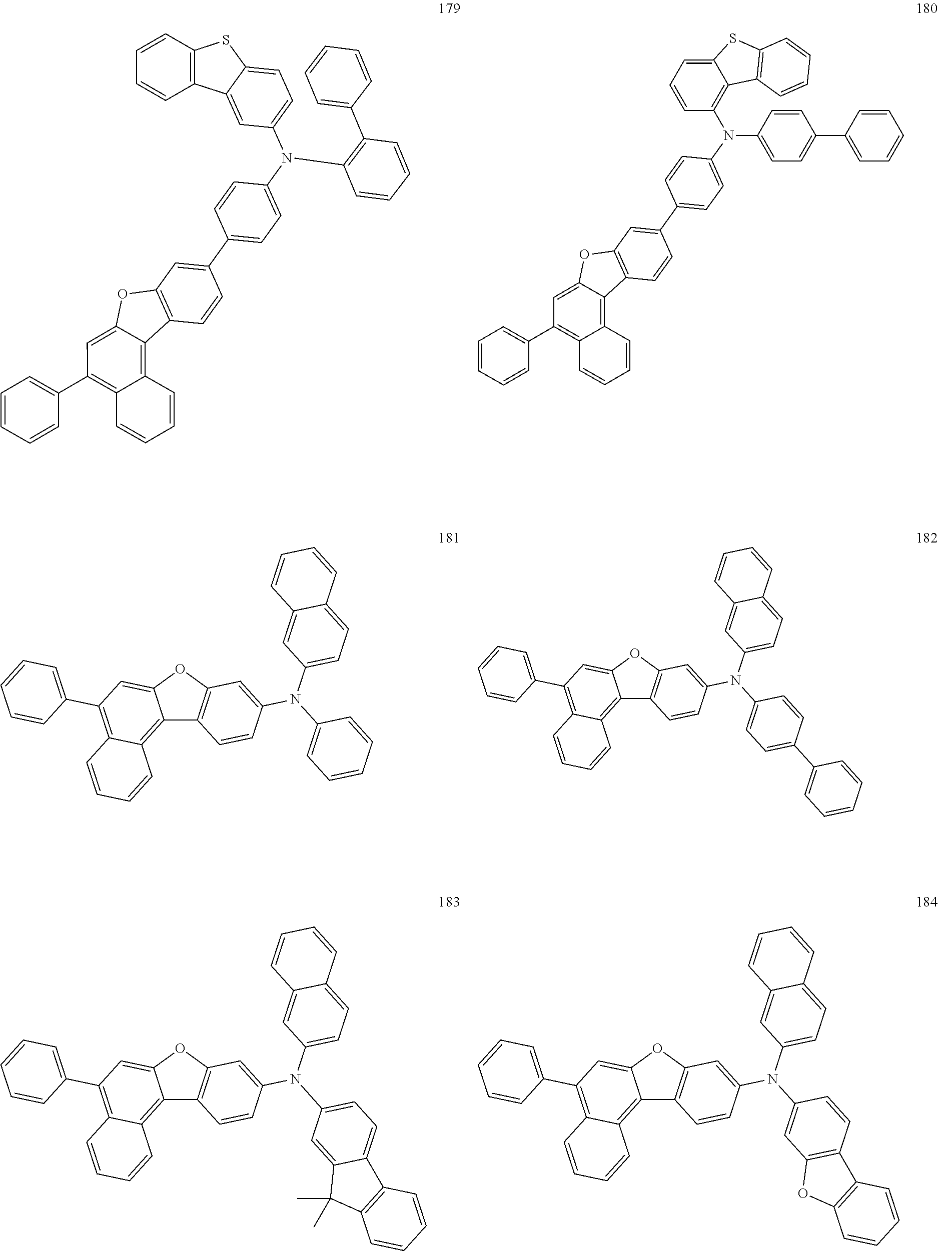 [SMILES: c1ccc(-c2ccccc2N(c2ccc(-c3ccc4c(c3)oc3cc(-c5ccccc5)c5ccccc5c34)cc2)c2ccc3sc4ccccc4c3c2)cc1 ; c1ccc(-c2ccc(N(c3ccc(-c4ccc5c(c4)oc4cc(-c6ccccc6)c6ccccc6c45)cc3)c3cccc4sc5ccccc5c34)cc2)cc1 ; c1ccc(-c2cc3oc4cc(N(c5ccccc5)c5ccc6ccccc6c5)ccc4c3c3ccccc23)cc1 ; c1ccc(-c2ccc(N(c3ccc4ccccc4c3)c3ccc4c(c3)oc3cc(-c5ccccc5)c5ccccc5c34)cc2)cc1 ; CC1(C)c2ccccc2-c2ccc(N(c3ccc4ccccc4c3)c3ccc4c(c3)oc3cc(-c5ccccc5)c5ccccc5c34)cc21 ; c1ccc(-c2cc3oc4cc(N(c5ccc6ccccc6c5)c5ccc6c(c5)oc5ccccc56)ccc4c3c3ccccc23)cc1]

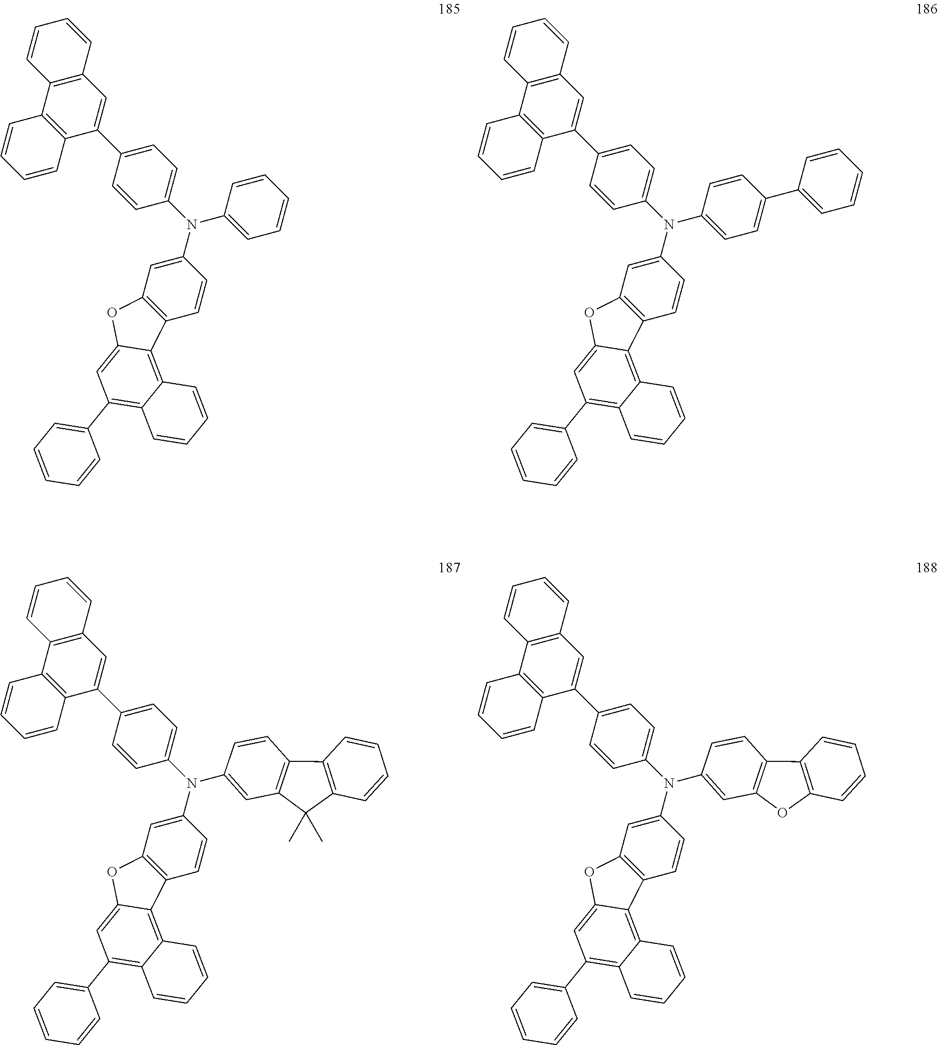 [SMILES: c1ccc(-c2cc3oc4cc(N(c5ccccc5)c5ccc(-c6cc7ccccc7c7ccccc67)cc5)ccc4c3c3ccccc23)cc1 ; c1ccc(-c2ccc(N(c3ccc(-c4cc5ccccc5c5ccccc45)cc3)c3ccc4c(c3)oc3cc(-c5ccccc5)c5ccccc5c34)cc2)cc1 ; CC1(C)c2ccccc2-c2ccc(N(c3ccc(-c4cc5ccccc5c5ccccc45)cc3)c3ccc4c(c3)oc3cc(-c5ccccc5)c5ccccc5c34)cc21 ; c1ccc(-c2cc3oc4cc(N(c5ccc(-c6cc7ccccc7c7ccccc67)cc5)c5ccc6c(c5)oc5ccccc56)ccc4c3c3ccccc23)cc1]

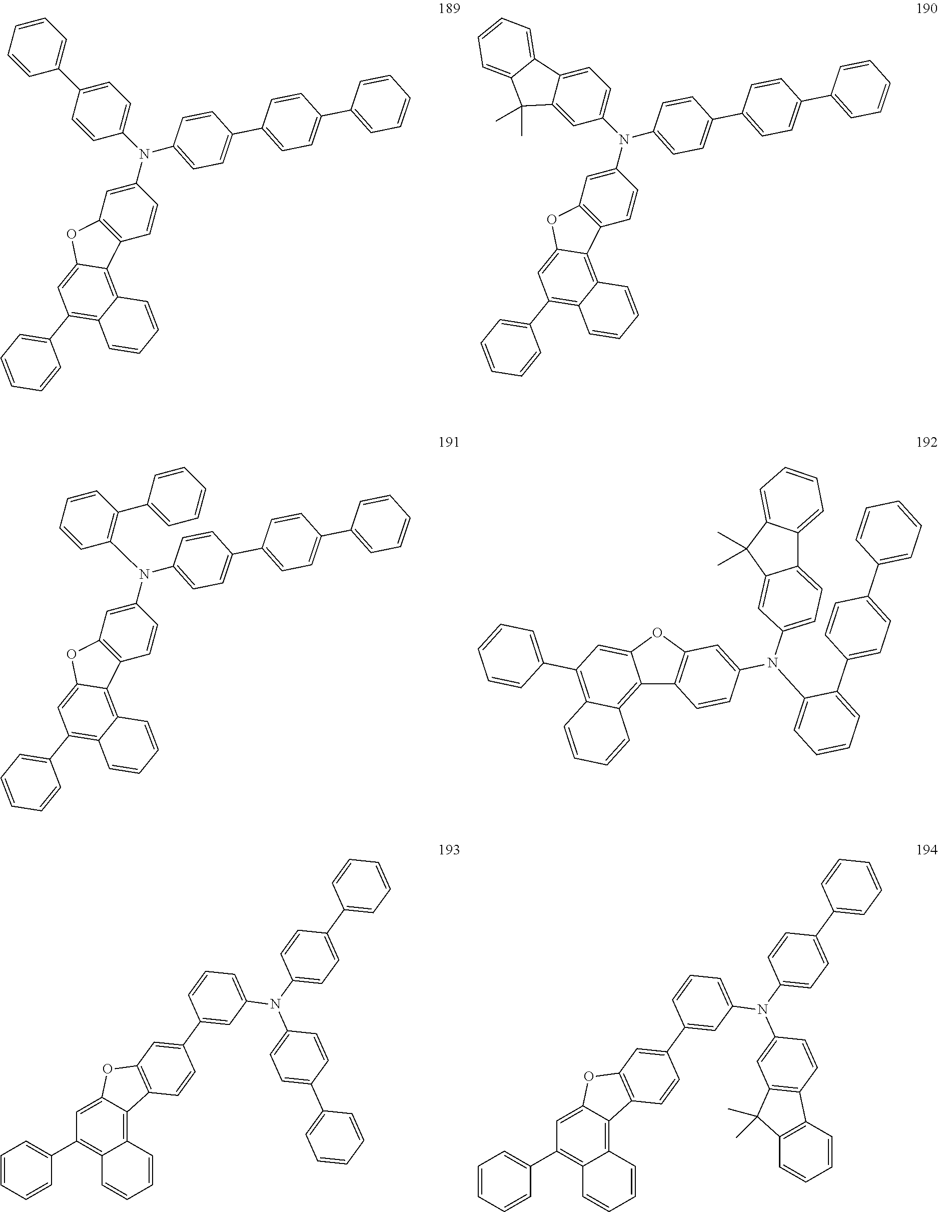

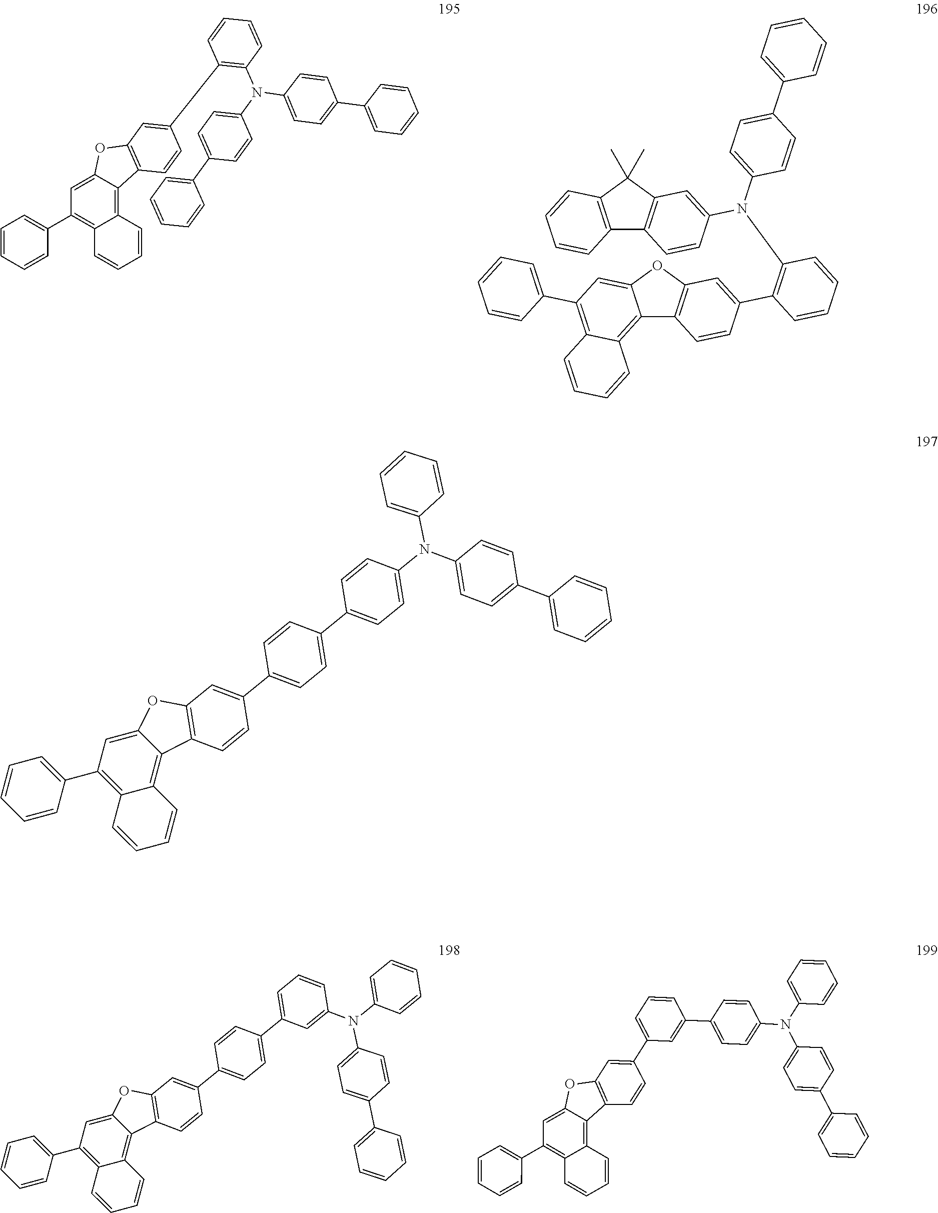 [SMILES: c1ccc(-c2ccc(N(c3ccc(-c4ccccc4)cc3)c3ccccc3-c3ccc4c(c3)oc3cc(-c5ccccc5)c5ccccc5c34)cc2)cc1 ; CC1(C)c2ccccc2-c2ccc(N(c3ccc(-c4ccccc4)cc3)c3ccccc3-c3ccc4c(c3)oc3cc(-c5ccccc5)c5ccccc5c34)cc21 ; c1ccc(-c2ccc(N(c3ccccc3)c3ccc(-c4ccc(-c5ccc6c(c5)oc5cc(-c7ccccc7)c7ccccc7c56)cc4)cc3)cc2)cc1 ; c1ccc(-c2ccc(N(c3ccccc3)c3cccc(-c4ccc(-c5ccc6c(c5)oc5cc(-c7ccccc7)c7ccccc7c56)cc4)c3)cc2)cc1 ; c1ccc(-c2ccc(N(c3ccccc3)c3ccc(-c4cccc(-c5ccc6c(c5)oc5cc(-c7ccccc7)c7ccccc7c56)c4)cc3)cc2)cc1]

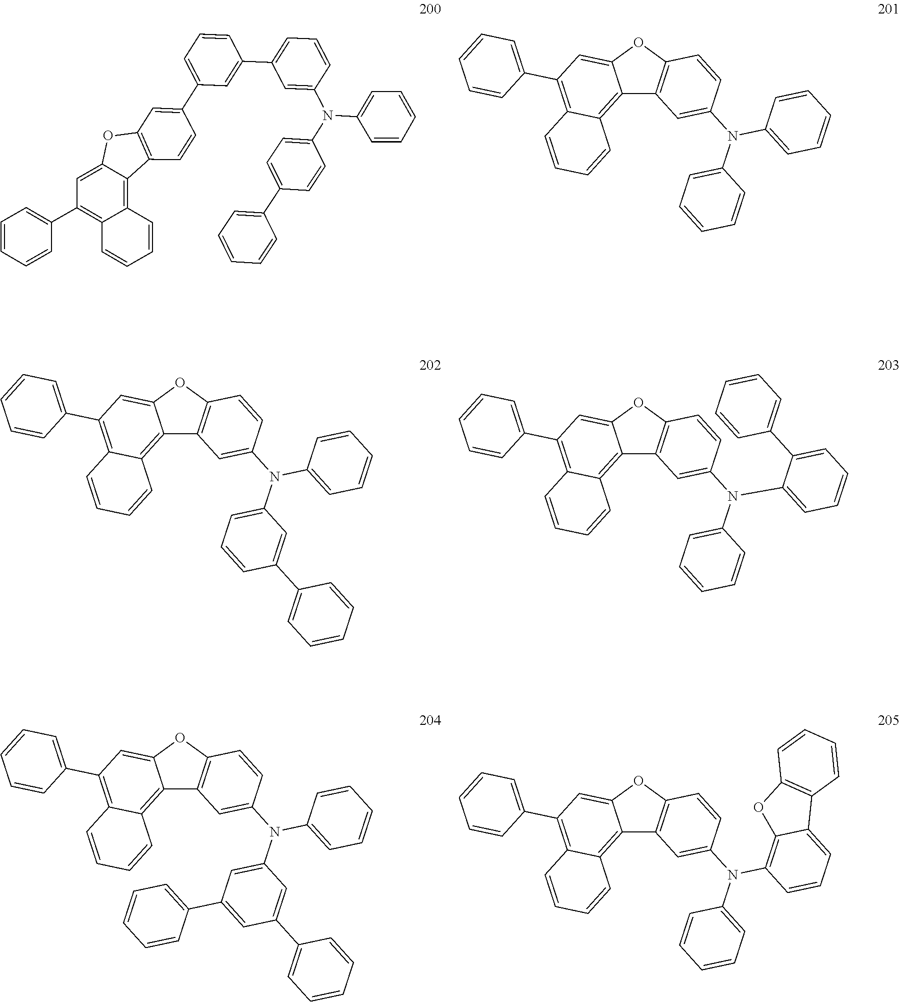 [SMILES: c1ccc(-c2ccc(N(c3ccccc3)c3cccc(-c4cccc(-c5ccc6c(c5)oc5cc(-c7ccccc7)c7ccccc7c56)c4)c3)cc2)cc1 ; c1ccc(-c2cc3oc4ccc(N(c5ccccc5)c5ccccc5)cc4c3c3ccccc23)cc1 ; c1ccc(-c2cccc(N(c3ccccc3)c3ccc4oc5cc(-c6ccccc6)c6ccccc6c5c4c3)c2)cc1 ; c1ccc(-c2ccccc2N(c2ccccc2)c2ccc3oc4cc(-c5ccccc5)c5ccccc5c4c3c2)cc1 ; c1ccc(-c2cc(-c3ccccc3)cc(N(c3ccccc3)c3ccc4oc5cc(-c6ccccc6)c6ccccc6c5c4c3)c2)cc1 ; c1ccc(-c2cc3oc4ccc(N(c5ccccc5)c5cccc6c5oc5ccccc56)cc4c3c3ccccc23)cc1]

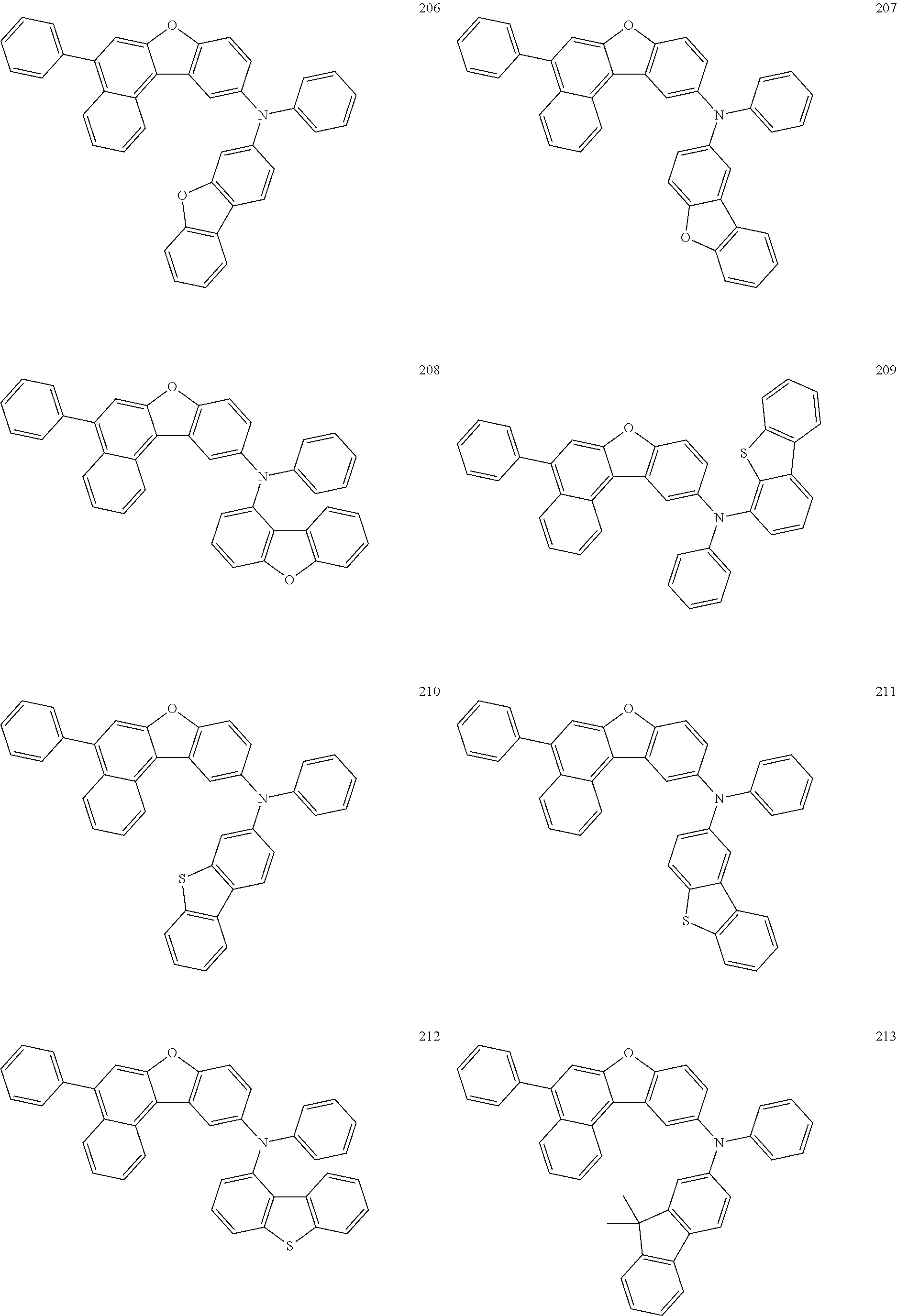

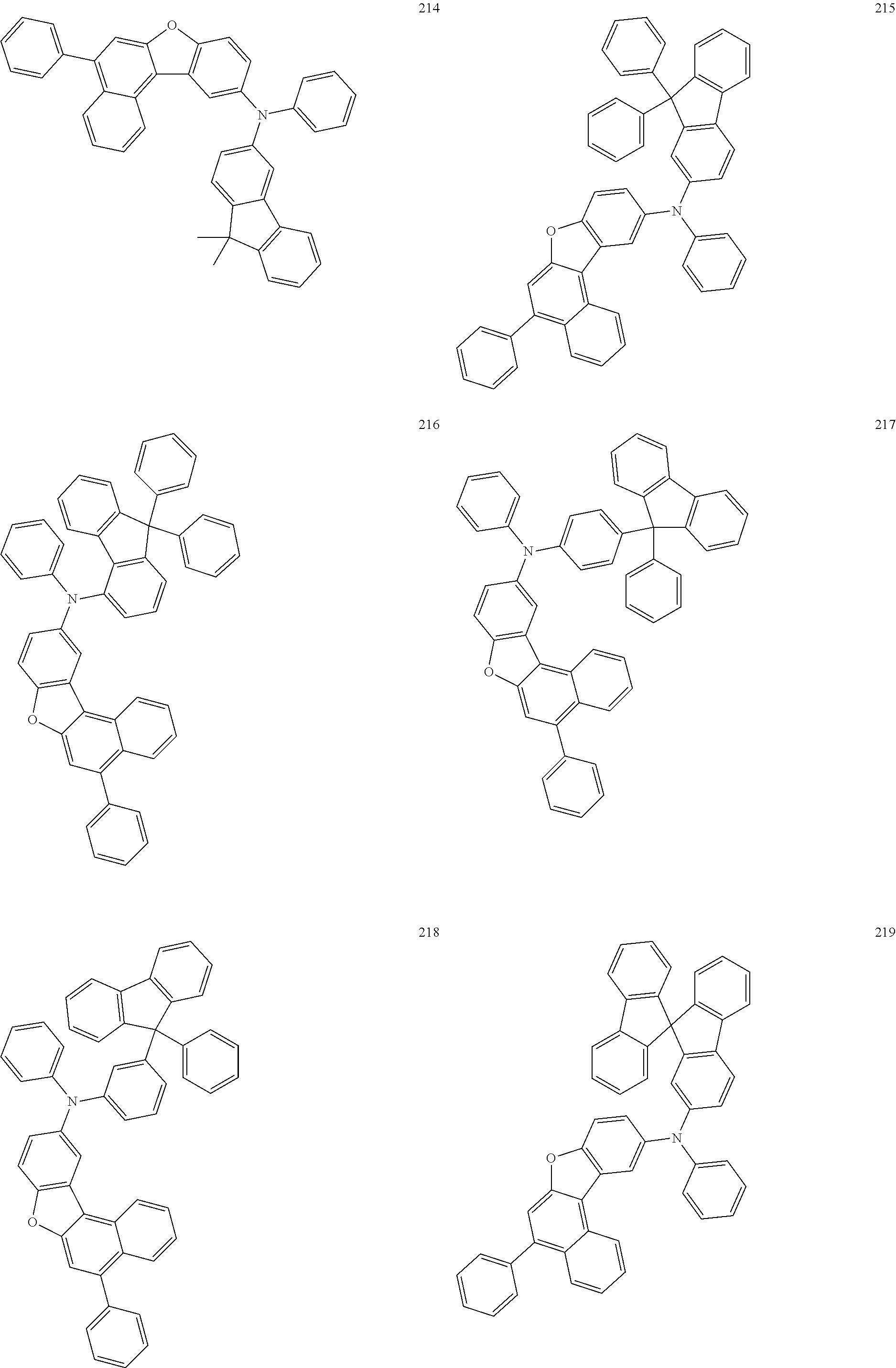

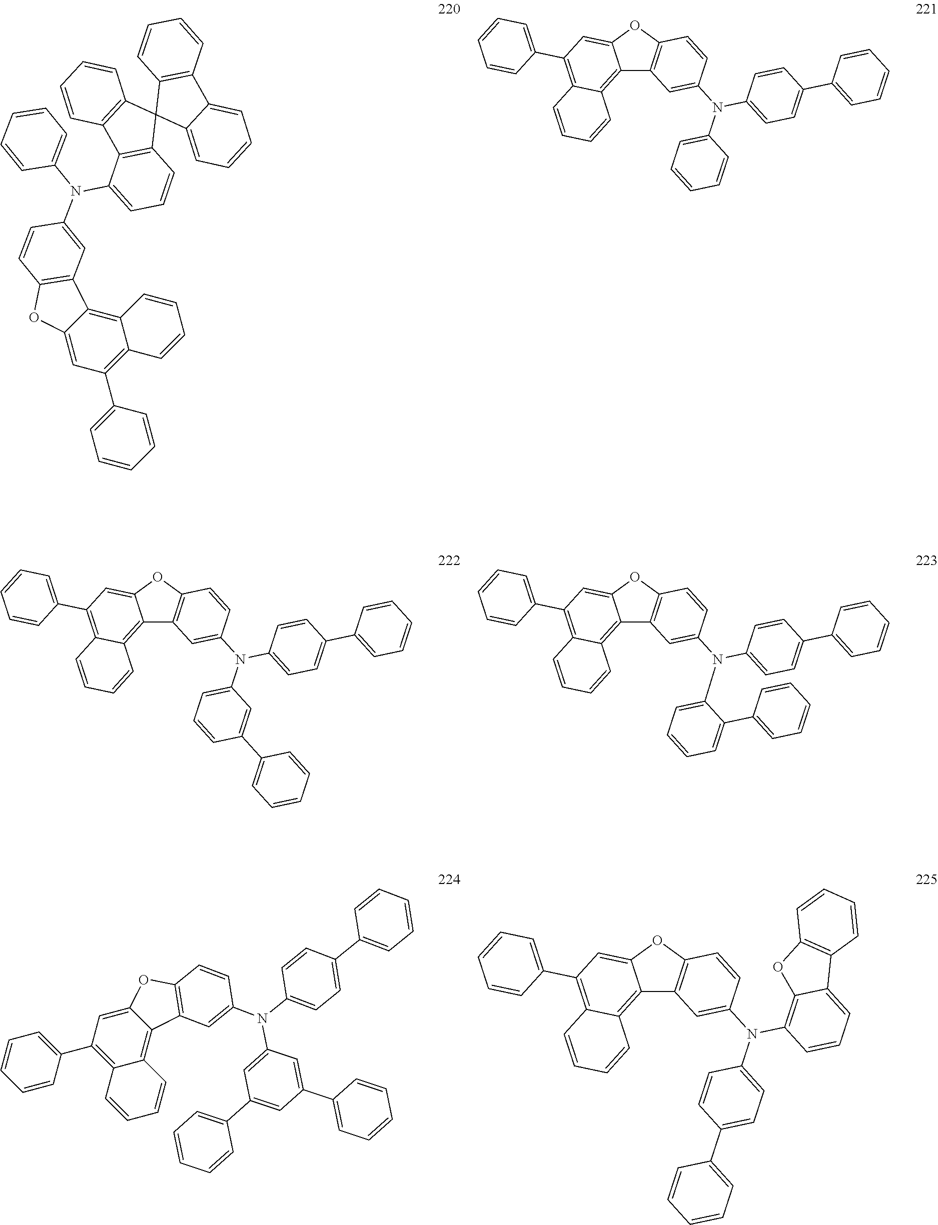 [SMILES: c1ccc(-c2cc3oc4ccc(N(c5ccccc5)c5cccc6c5-c5ccccc5C65c6ccccc6-c6ccccc65)cc4c3c3ccccc23)cc1 ; c1ccc(-c2ccc(N(c3ccccc3)c3ccc4oc5cc(-c6ccccc6)c6ccccc6c5c4c3)cc2)cc1 ; c1ccc(-c2ccc(N(c3cccc(-c4ccccc4)c3)c3ccc4oc5cc(-c6ccccc6)c6ccccc6c5c4c3)cc2)cc1 ; c1ccc(-c2ccc(N(c3ccc4oc5cc(-c6ccccc6)c6ccccc6c5c4c3)c3ccccc3-c3ccccc3)cc2)cc1 ; c1ccc(-c2ccc(N(c3cc(-c4ccccc4)cc(-c4ccccc4)c3)c3ccc4oc5cc(-c6ccccc6)c6ccccc6c5c4c3)cc2)cc1 ; c1ccc(-c2ccc(N(c3ccc4oc5cc(-c6ccccc6)c6ccccc6c5c4c3)c3cccc4c3oc3ccccc34)cc2)cc1]

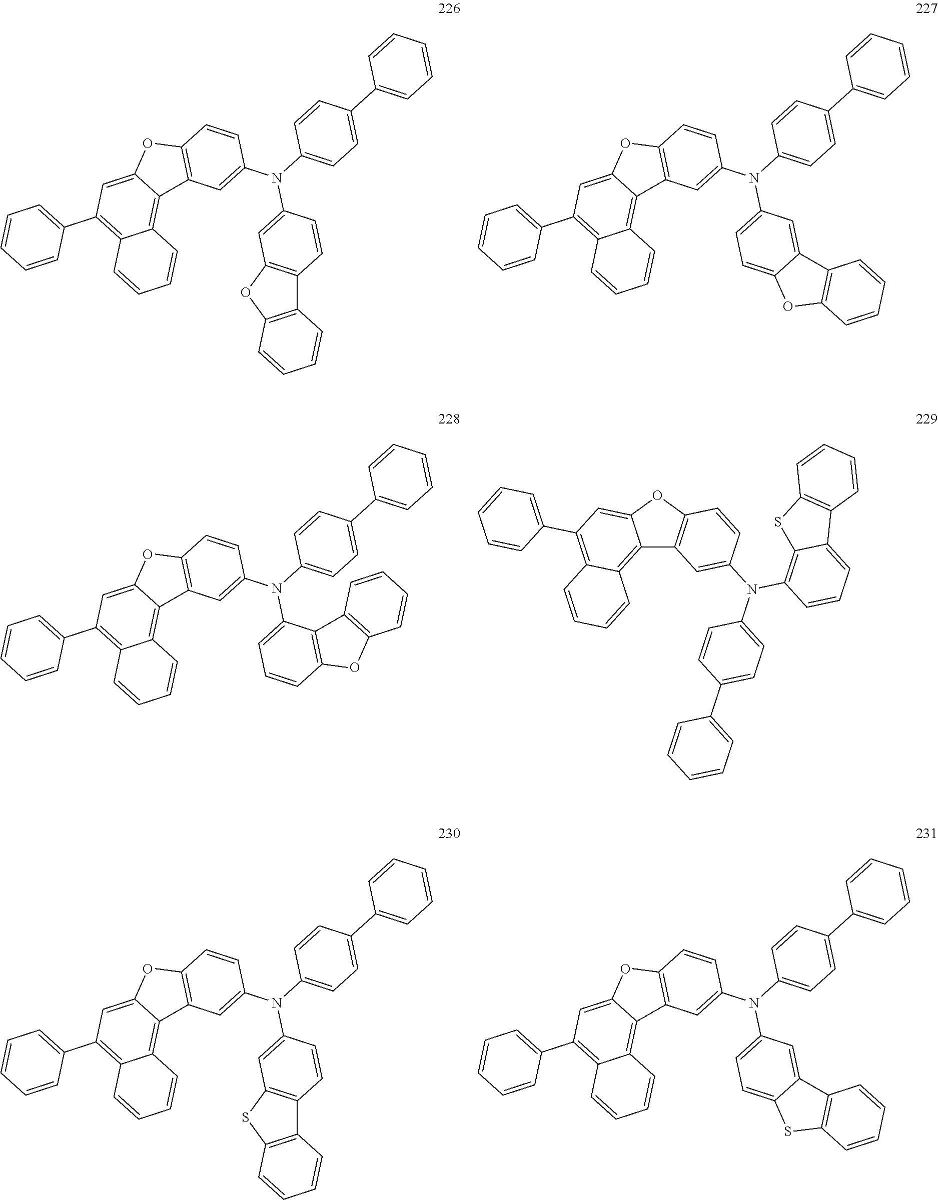 [SMILES: c1ccc(-c2ccc(N(c3ccc4c(c3)oc3ccccc34)c3ccc4oc5cc(-c6ccccc6)c6ccccc6c5c4c3)cc2)cc1 ; c1ccc(-c2ccc(N(c3ccc4oc5ccccc5c4c3)c3ccc4oc5cc(-c6ccccc6)c6ccccc6c5c4c3)cc2)cc1 ; c1ccc(-c2ccc(N(c3ccc4oc5cc(-c6ccccc6)c6ccccc6c5c4c3)c3cccc4oc5ccccc5c34)cc2)cc1 ; c1ccc(-c2ccc(N(c3ccc4oc5cc(-c6ccccc6)c6ccccc6c5c4c3)c3cccc4c3sc3ccccc34)cc2)cc1 ; c1ccc(-c2ccc(N(c3ccc4c(c3)sc3ccccc34)c3ccc4oc5cc(-c6ccccc6)c6ccccc6c5c4c3)cc2)cc1 ; c1ccc(-c2ccc(N(c3ccc4sc5ccccc5c4c3)c3ccc4oc5cc(-c6ccccc6)c6ccccc6c5c4c3)cc2)cc1]

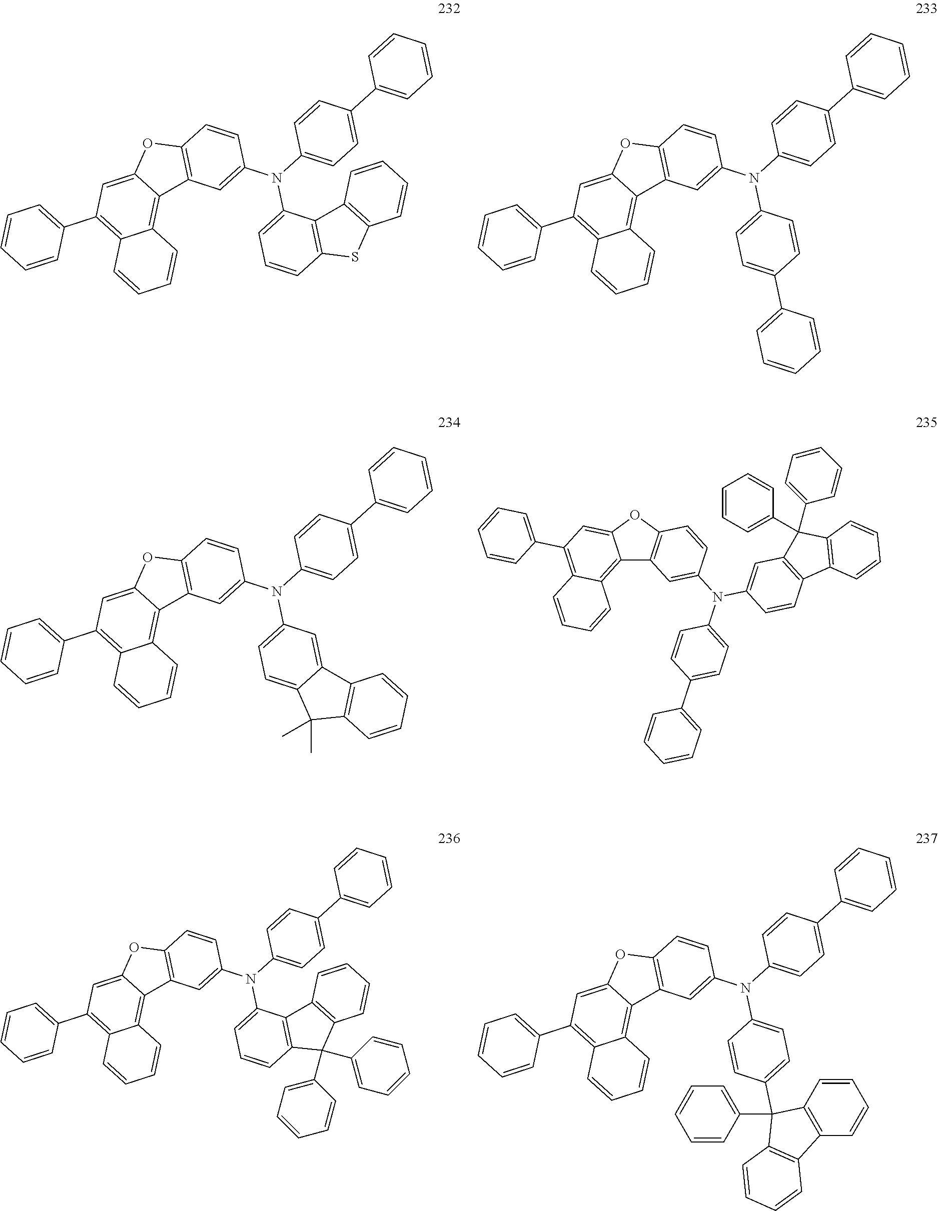

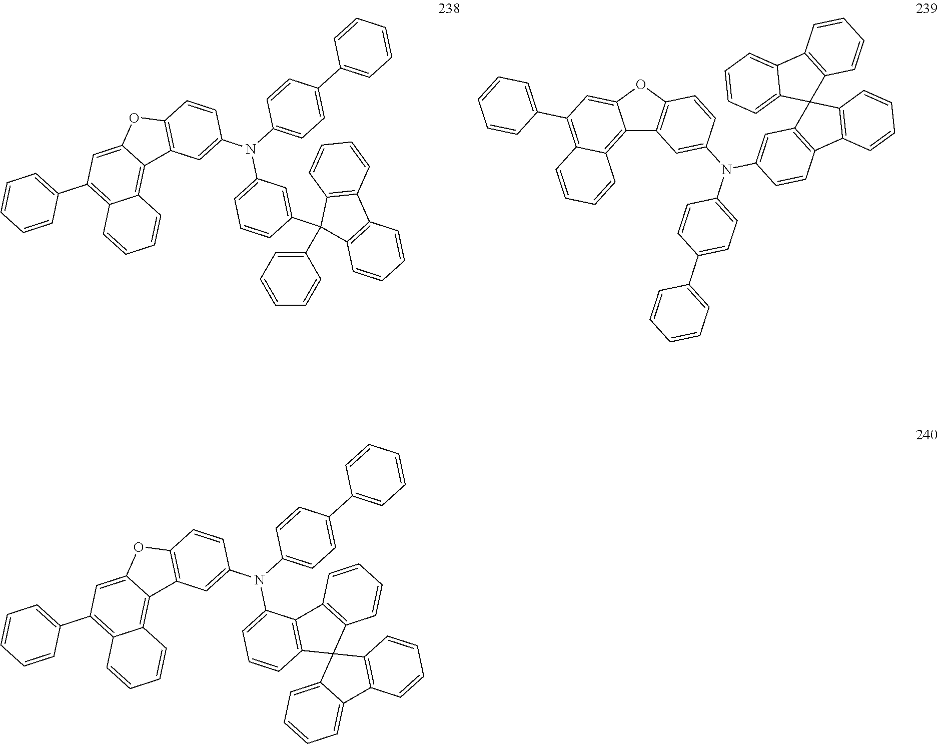 [SMILES: c1ccc(-c2ccc(N(c3cccc(C4(c5ccccc5)c5ccccc5-c5ccccc54)c3)c3ccc4oc5cc(-c6ccccc6)c6ccccc6c5c4c3)cc2)cc1 ; c1ccc(-c2ccc(N(c3ccc4c(c3)C3(c5ccccc5-c5ccccc53)c3ccccc3-4)c3ccc4oc5cc(-c6ccccc6)c6ccccc6c5c4c3)cc2)cc1 ; c1ccc(-c2ccc(N(c3ccc4oc5cc(-c6ccccc6)c6ccccc6c5c4c3)c3cccc4c3-c3ccccc3C43c4ccccc4-c4ccccc43)cc2)cc1]

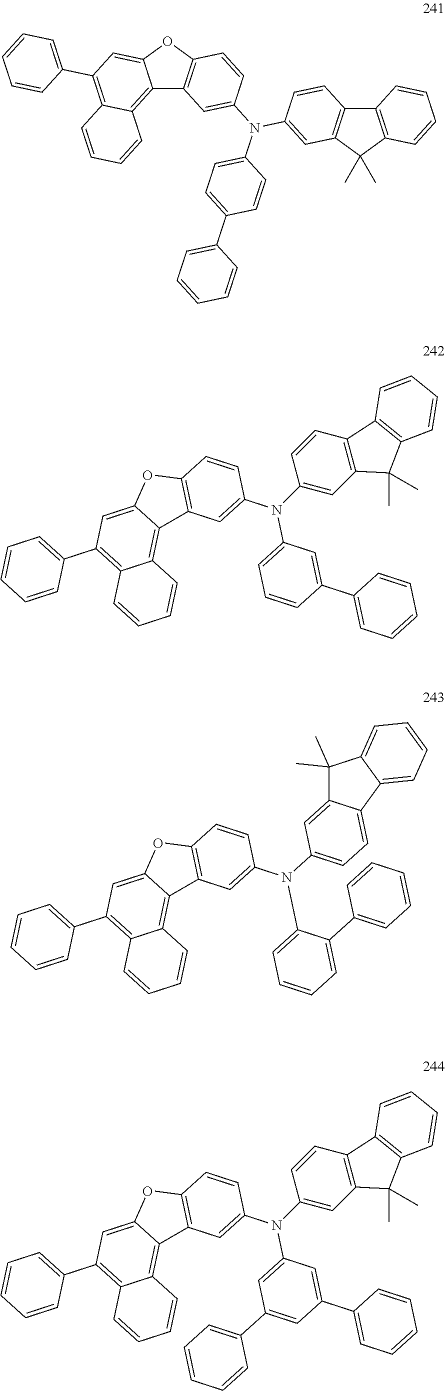 [SMILES: CC1(C)c2ccccc2-c2ccc(N(c3ccc(-c4ccccc4)cc3)c3ccc4oc5cc(-c6ccccc6)c6ccccc6c5c4c3)cc21 ; CC1(C)c2ccccc2-c2ccc(N(c3cccc(-c4ccccc4)c3)c3ccc4oc5cc(-c6ccccc6)c6ccccc6c5c4c3)cc21 ; CC1(C)c2ccccc2-c2ccc(N(c3ccc4oc5cc(-c6ccccc6)c6ccccc6c5c4c3)c3ccccc3-c3ccccc3)cc21 ; CC1(C)c2ccccc2-c2ccc(N(c3cc(-c4ccccc4)cc(-c4ccccc4)c3)c3ccc4oc5cc(-c6ccccc6)c6ccccc6c5c4c3)cc21]

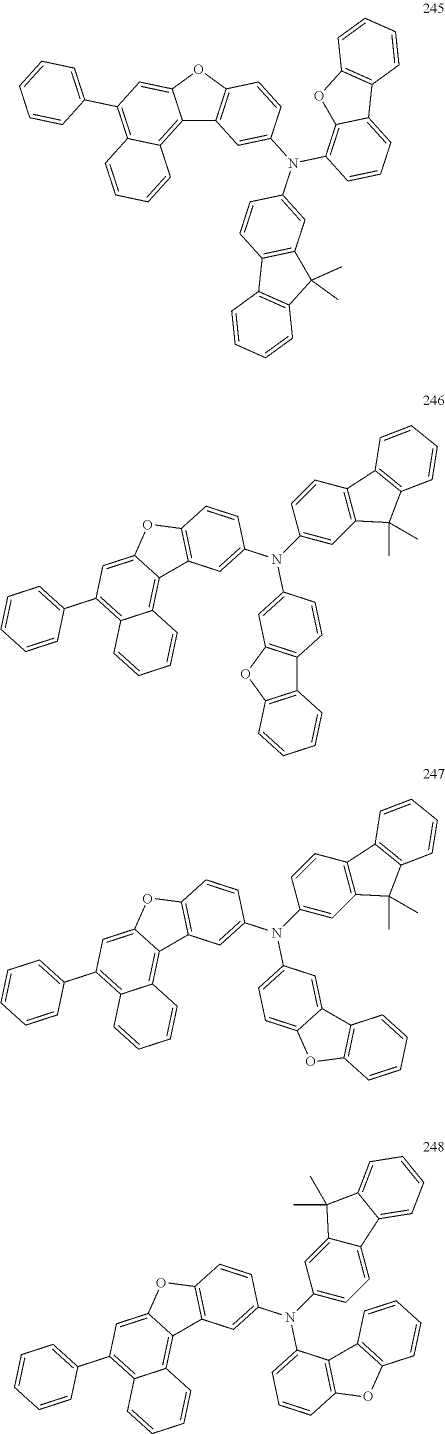 [SMILES: CC1(C)c2ccccc2-c2ccc(N(c3ccc4oc5cc(-c6ccccc6)c6ccccc6c5c4c3)c3cccc4c3oc3ccccc34)cc21 ; CC1(C)c2ccccc2-c2ccc(N(c3ccc4c(c3)oc3ccccc34)c3ccc4oc5cc(-c6ccccc6)c6ccccc6c5c4c3)cc21 ; CC1(C)c2ccccc2-c2ccc(N(c3ccc4oc5ccccc5c4c3)c3ccc4oc5cc(-c6ccccc6)c6ccccc6c5c4c3)cc21 ; CC1(C)c2ccccc2-c2ccc(N(c3ccc4oc5cc(-c6ccccc6)c6ccccc6c5c4c3)c3cccc4oc5ccccc5c34)cc21]

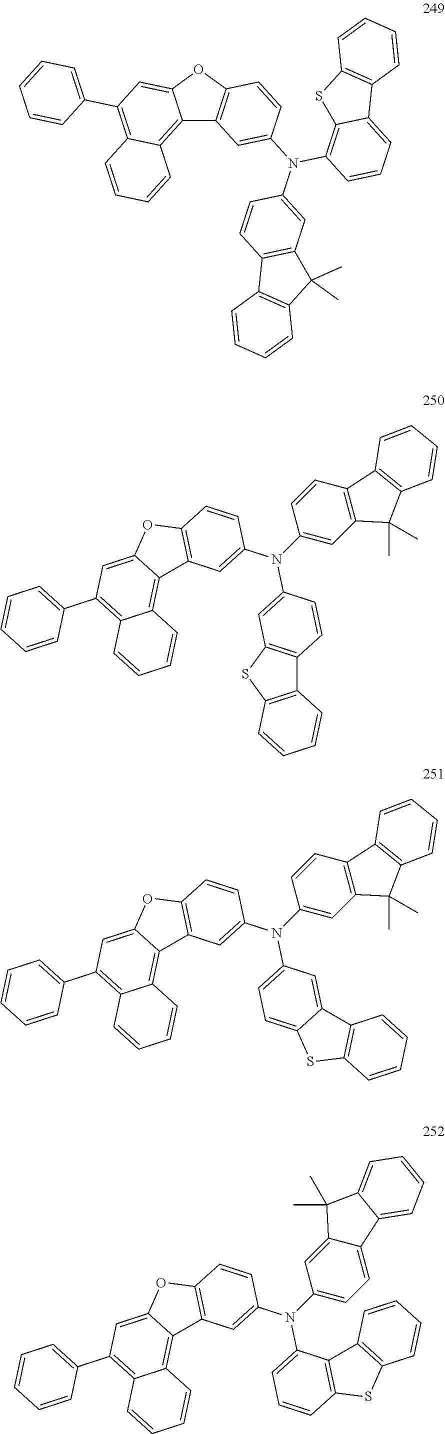 [SMILES: CC1(C)c2ccccc2-c2ccc(N(c3ccc4oc5cc(-c6ccccc6)c6ccccc6c5c4c3)c3cccc4c3sc3ccccc34)cc21 ; CC1(C)c2ccccc2-c2ccc(N(c3ccc4c(c3)sc3ccccc34)c3ccc4oc5cc(-c6ccccc6)c6ccccc6c5c4c3)cc21 ; CC1(C)c2ccccc2-c2ccc(N(c3ccc4sc5ccccc5c4c3)c3ccc4oc5cc(-c6ccccc6)c6ccccc6c5c4c3)cc21 ; CC1(C)c2ccccc2-c2ccc(N(c3ccc4oc5cc(-c6ccccc6)c6ccccc6c5c4c3)c3cccc4sc5ccccc5c34)cc21]

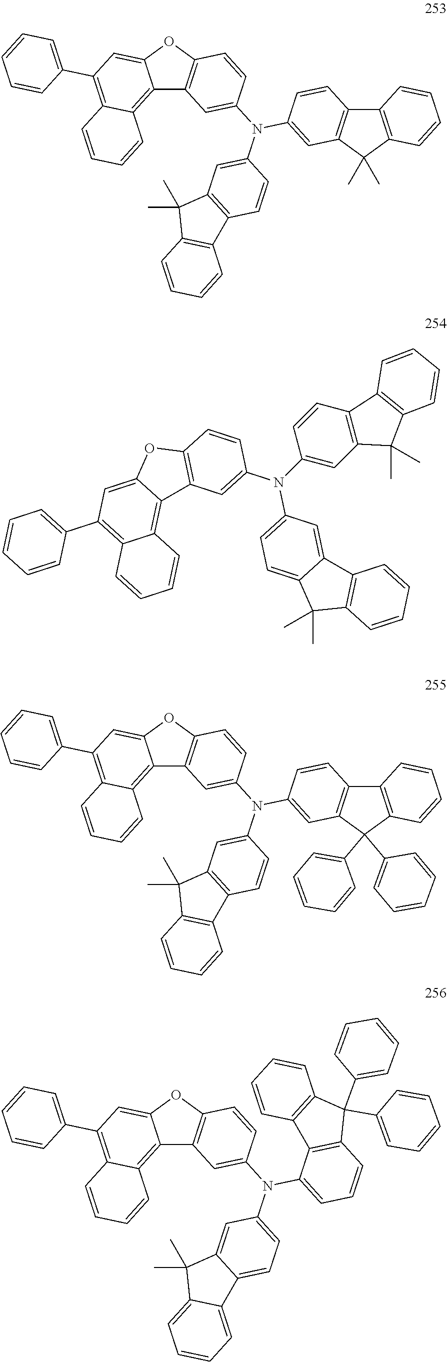 [SMILES: CC1(C)c2ccccc2-c2ccc(N(c3ccc4c(c3)C(C)(C)c3ccccc3-4)c3ccc4oc5cc(-c6ccccc6)c6ccccc6c5c4c3)cc21 ; CC1(C)c2ccccc2-c2cc(N(c3ccc4c(c3)C(C)(C)c3ccccc3-4)c3ccc4oc5cc(-c6ccccc6)c6ccccc6c5c4c3)ccc21 ; CC1(C)c2ccccc2-c2ccc(N(c3ccc4c(c3)C(c3ccccc3)(c3ccccc3)c3ccccc3-4)c3ccc4oc5cc(-c6ccccc6)c6ccccc6c5c4c3)cc21 ; CC1(C)c2ccccc2-c2ccc(N(c3ccc4oc5cc(-c6ccccc6)c6ccccc6c5c4c3)c3cccc4c3-c3ccccc3C4(c3ccccc3)c3ccccc3)cc21]

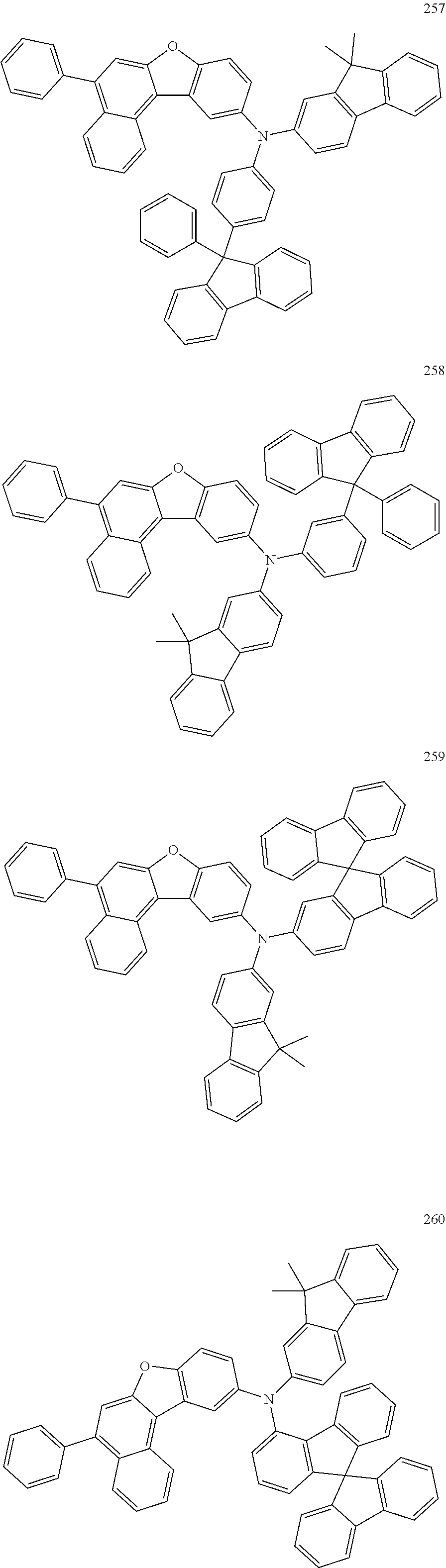 [SMILES: CC1(C)c2ccccc2-c2ccc(N(c3ccc(C4(c5ccccc5)c5ccccc5-c5ccccc54)cc3)c3ccc4oc5cc(-c6ccccc6)c6ccccc6c5c4c3)cc21 ; CC1(C)c2ccccc2-c2ccc(N(c3cccc(C4(c5ccccc5)c5ccccc5-c5ccccc54)c3)c3ccc4oc5cc(-c6ccccc6)c6ccccc6c5c4c3)cc21 ; CC1(C)c2ccccc2-c2ccc(N(c3ccc4c(c3)C3(c5ccccc5-c5ccccc53)c3ccccc3-4)c3ccc4oc5cc(-c6ccccc6)c6ccccc6c5c4c3)cc21 ; CC1(C)c2ccccc2-c2ccc(N(c3ccc4oc5cc(-c6ccccc6)c6ccccc6c5c4c3)c3cccc4c3-c3ccccc3C43c4ccccc4-c4ccccc43)cc21]

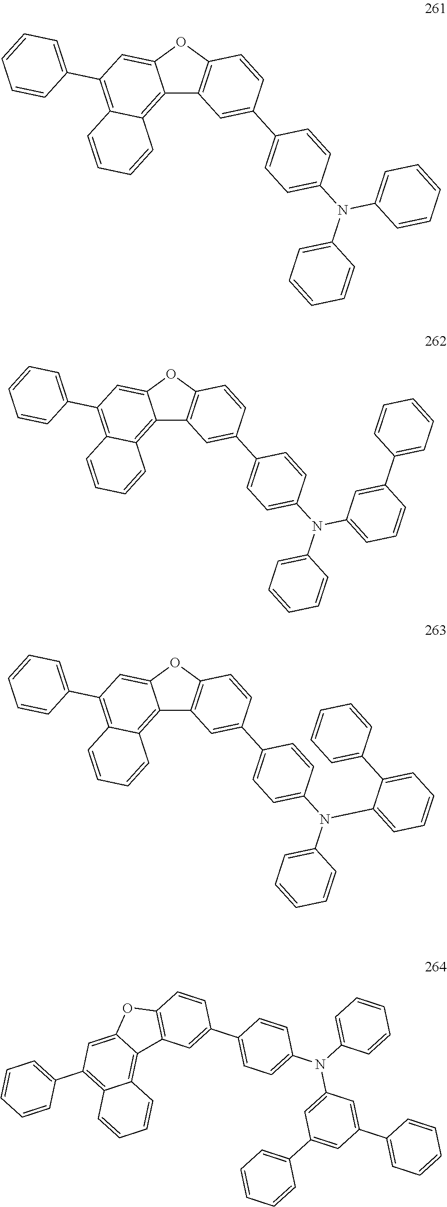 [SMILES: c1ccc(-c2cc3oc4ccc(-c5ccc(N(c6ccccc6)c6ccccc6)cc5)cc4c3c3ccccc23)cc1 ; c1ccc(-c2cccc(N(c3ccccc3)c3ccc(-c4ccc5oc6cc(-c7ccccc7)c7ccccc7c6c5c4)cc3)c2)cc1 ; c1ccc(-c2ccccc2N(c2ccccc2)c2ccc(-c3ccc4oc5cc(-c6ccccc6)c6ccccc6c5c4c3)cc2)cc1 ; c1ccc(-c2cc(-c3ccccc3)cc(N(c3ccccc3)c3ccc(-c4ccc5oc6cc(-c7ccccc7)c7ccccc7c6c5c4)cc3)c2)cc1]

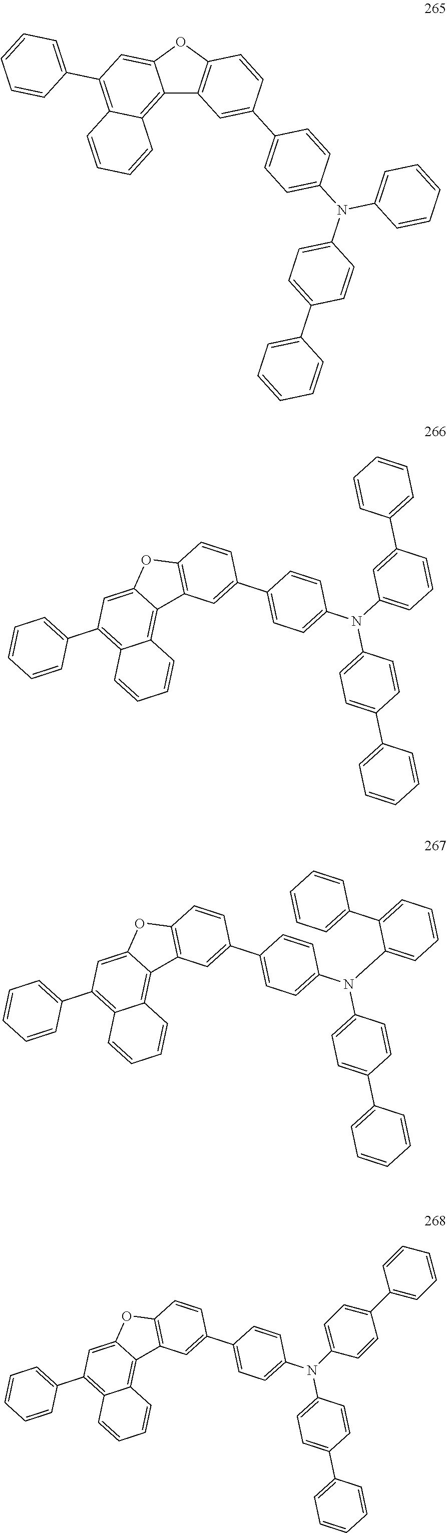 [SMILES: c1ccc(-c2ccc(N(c3ccccc3)c3ccc(-c4ccc5oc6cc(-c7ccccc7)c7ccccc7c6c5c4)cc3)cc2)cc1 ; c1ccc(-c2ccc(N(c3ccc(-c4ccc5oc6cc(-c7ccccc7)c7ccccc7c6c5c4)cc3)c3cccc(-c4ccccc4)c3)cc2)cc1 ; c1ccc(-c2ccc(N(c3ccc(-c4ccc5oc6cc(-c7ccccc7)c7ccccc7c6c5c4)cc3)c3ccccc3-c3ccccc3)cc2)cc1 ; c1ccc(-c2ccc(N(c3ccc(-c4ccccc4)cc3)c3ccc(-c4ccc5oc6cc(-c7ccccc7)c7ccccc7c6c5c4)cc3)cc2)cc1]

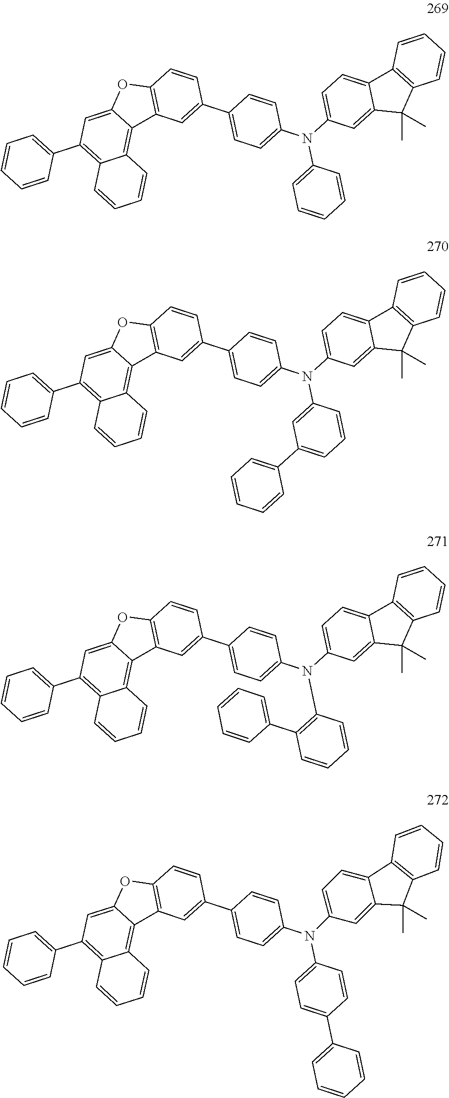 [SMILES: CC1(C)c2ccccc2-c2ccc(N(c3ccccc3)c3ccc(-c4ccc5oc6cc(-c7ccccc7)c7ccccc7c6c5c4)cc3)cc21 ; CC1(C)c2ccccc2-c2ccc(N(c3ccc(-c4ccc5oc6cc(-c7ccccc7)c7ccccc7c6c5c4)cc3)c3cccc(-c4ccccc4)c3)cc21 ; CC1(C)c2ccccc2-c2ccc(N(c3ccc(-c4ccc5oc6cc(-c7ccccc7)c7ccccc7c6c5c4)cc3)c3ccccc3-c3ccccc3)cc21 ; CC1(C)c2ccccc2-c2ccc(N(c3ccc(-c4ccccc4)cc3)c3ccc(-c4ccc5oc6cc(-c7ccccc7)c7ccccc7c6c5c4)cc3)cc21]

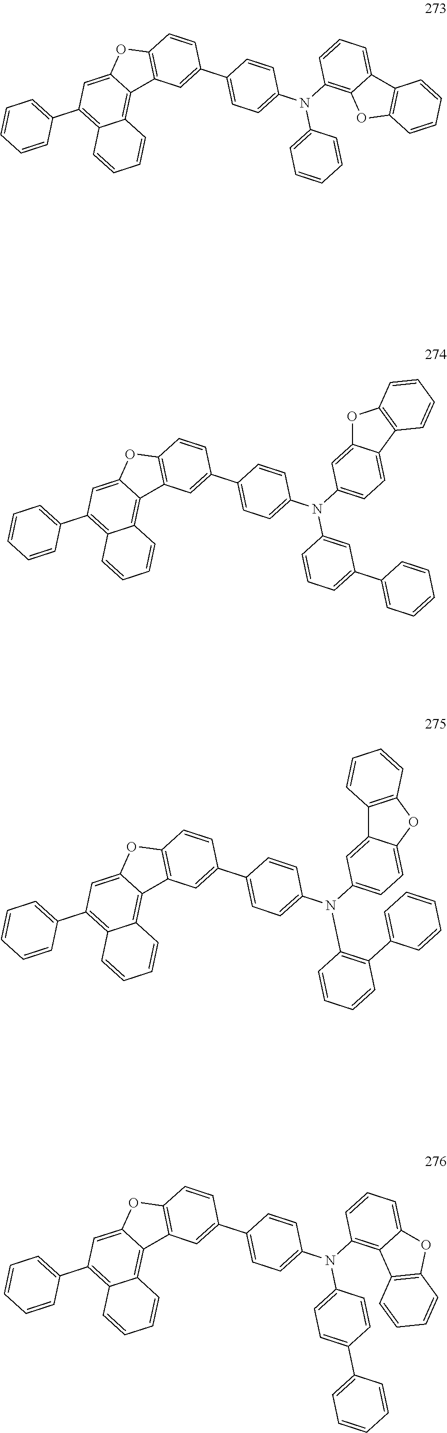 [SMILES: c1ccc(-c2cc3oc4ccc(-c5ccc(N(c6ccccc6)c6cccc7c6oc6ccccc67)cc5)cc4c3c3ccccc23)cc1 ; c1ccc(-c2cccc(N(c3ccc(-c4ccc5oc6cc(-c7ccccc7)c7ccccc7c6c5c4)cc3)c3ccc4c(c3)oc3ccccc34)c2)cc1 ; c1ccc(-c2ccccc2N(c2ccc(-c3ccc4oc5cc(-c6ccccc6)c6ccccc6c5c4c3)cc2)c2ccc3oc4ccccc4c3c2)cc1 ; c1ccc(-c2ccc(N(c3ccc(-c4ccc5oc6cc(-c7ccccc7)c7ccccc7c6c5c4)cc3)c3cccc4oc5ccccc5c34)cc2)cc1]

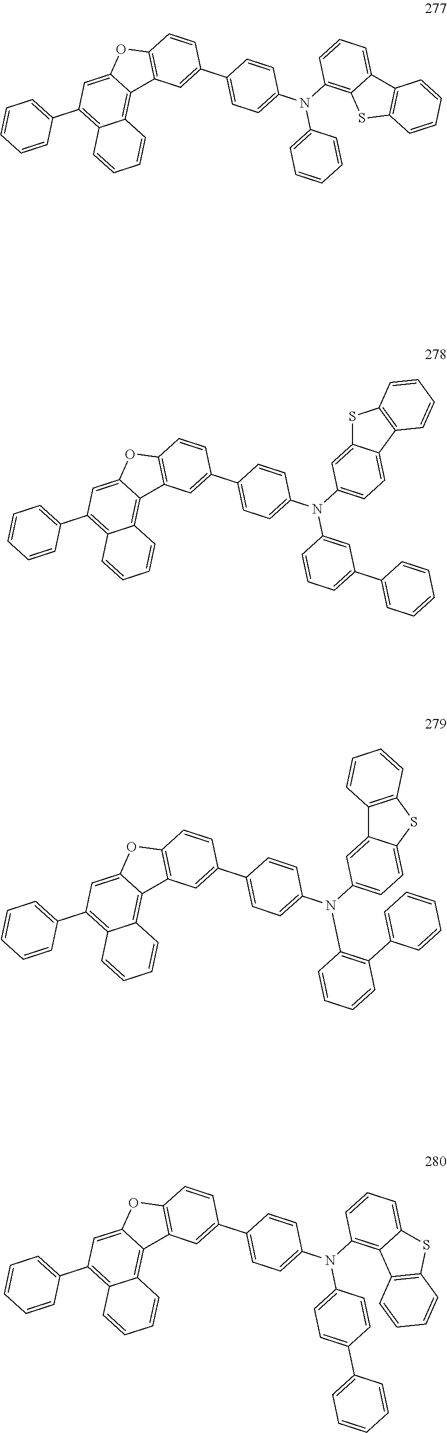 [SMILES: c1ccc(-c2cc3oc4ccc(-c5ccc(N(c6ccccc6)c6cccc7c6sc6ccccc67)cc5)cc4c3c3ccccc23)cc1 ; c1ccc(-c2cccc(N(c3ccc(-c4ccc5oc6cc(-c7ccccc7)c7ccccc7c6c5c4)cc3)c3ccc4c(c3)sc3ccccc34)c2)cc1 ; c1ccc(-c2ccccc2N(c2ccc(-c3ccc4oc5cc(-c6ccccc6)c6ccccc6c5c4c3)cc2)c2ccc3sc4ccccc4c3c2)cc1 ; c1ccc(-c2ccc(N(c3ccc(-c4ccc5oc6cc(-c7ccccc7)c7ccccc7c6c5c4)cc3)c3cccc4sc5ccccc5c34)cc2)cc1]

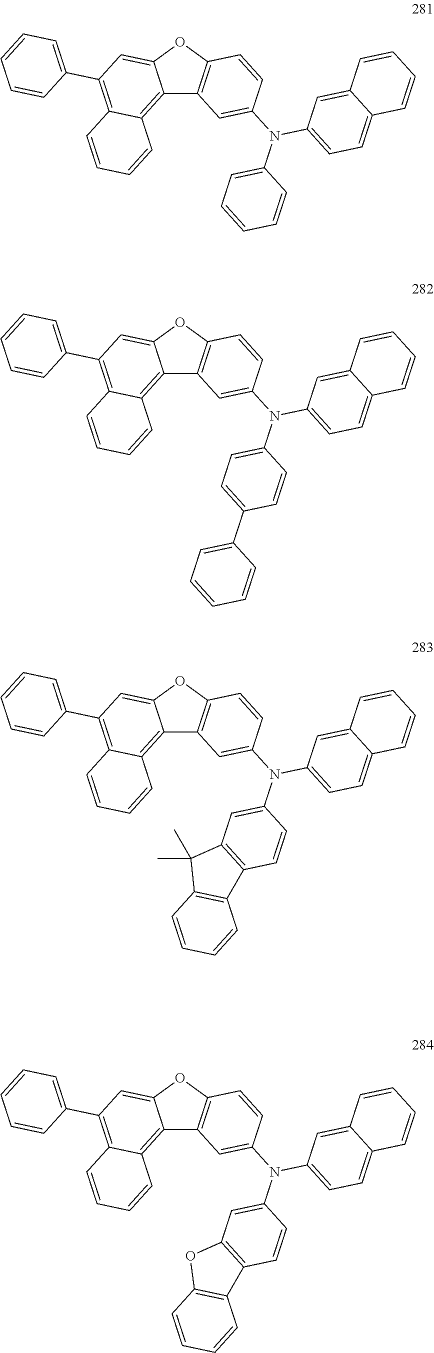 [SMILES: c1ccc(-c2cc3oc4ccc(N(c5ccccc5)c5ccc6ccccc6c5)cc4c3c3ccccc23)cc1 ; c1ccc(-c2ccc(N(c3ccc4ccccc4c3)c3ccc4oc5cc(-c6ccccc6)c6ccccc6c5c4c3)cc2)cc1 ; CC1(C)c2ccccc2-c2ccc(N(c3ccc4ccccc4c3)c3ccc4oc5cc(-c6ccccc6)c6ccccc6c5c4c3)cc21 ; c1ccc(-c2cc3oc4ccc(N(c5ccc6ccccc6c5)c5ccc6c(c5)oc5ccccc56)cc4c3c3ccccc23)cc1]

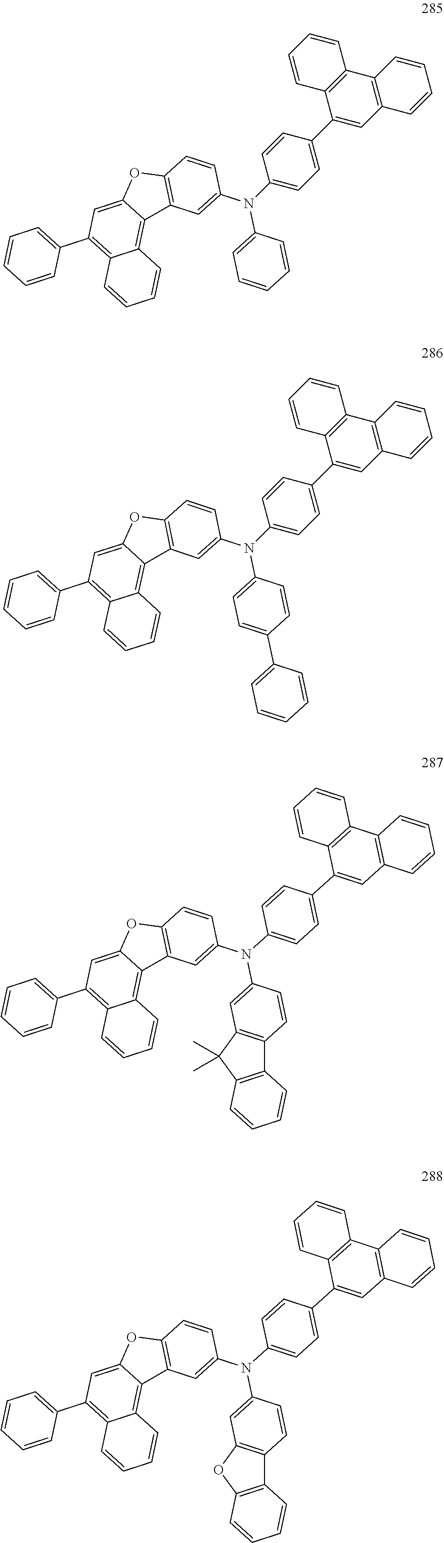 [SMILES: c1ccc(-c2cc3oc4ccc(N(c5ccccc5)c5ccc(-c6cc7ccccc7c7ccccc67)cc5)cc4c3c3ccccc23)cc1 ; c1ccc(-c2ccc(N(c3ccc(-c4cc5ccccc5c5ccccc45)cc3)c3ccc4oc5cc(-c6ccccc6)c6ccccc6c5c4c3)cc2)cc1 ; CC1(C)c2ccccc2-c2ccc(N(c3ccc(-c4cc5ccccc5c5ccccc45)cc3)c3ccc4oc5cc(-c6ccccc6)c6ccccc6c5c4c3)cc21 ; c1ccc(-c2cc3oc4ccc(N(c5ccc(-c6cc7ccccc7c7ccccc67)cc5)c5ccc6c(c5)oc5ccccc56)cc4c3c3ccccc23)cc1]

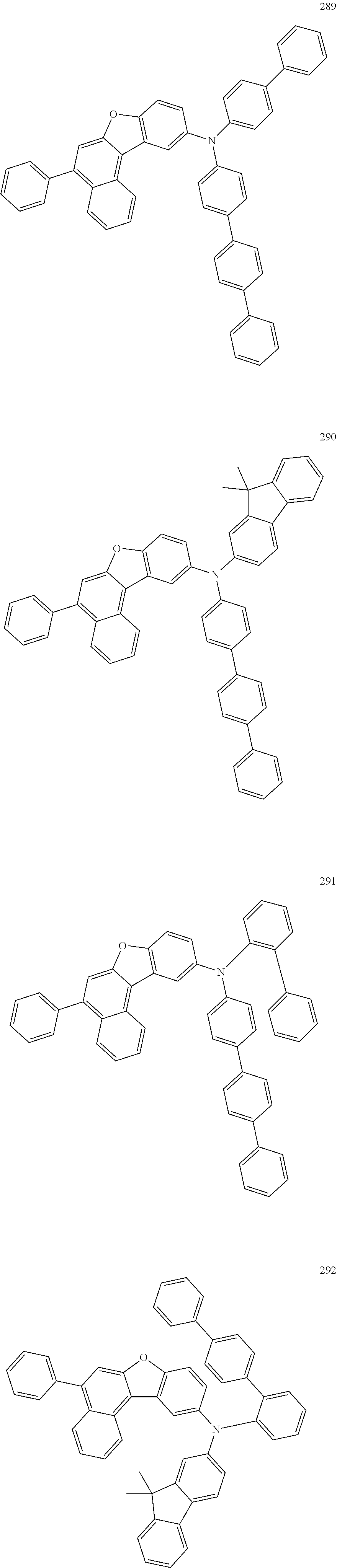 [SMILES: c1ccc(-c2ccc(-c3ccc(N(c4ccc(-c5ccccc5)cc4)c4ccc5oc6cc(-c7ccccc7)c7ccccc7c6c5c4)cc3)cc2)cc1 ; CC1(C)c2ccccc2-c2ccc(N(c3ccc(-c4ccc(-c5ccccc5)cc4)cc3)c3ccc4oc5cc(-c6ccccc6)c6ccccc6c5c4c3)cc21 ; c1ccc(-c2ccc(-c3ccc(N(c4ccc5oc6cc(-c7ccccc7)c7ccccc7c6c5c4)c4ccccc4-c4ccccc4)cc3)cc2)cc1 ; CC1(C)c2ccccc2-c2ccc(N(c3ccc4oc5cc(-c6ccccc6)c6ccccc6c5c4c3)c3ccccc3-c3ccc(-c4ccccc4)cc3)cc21]

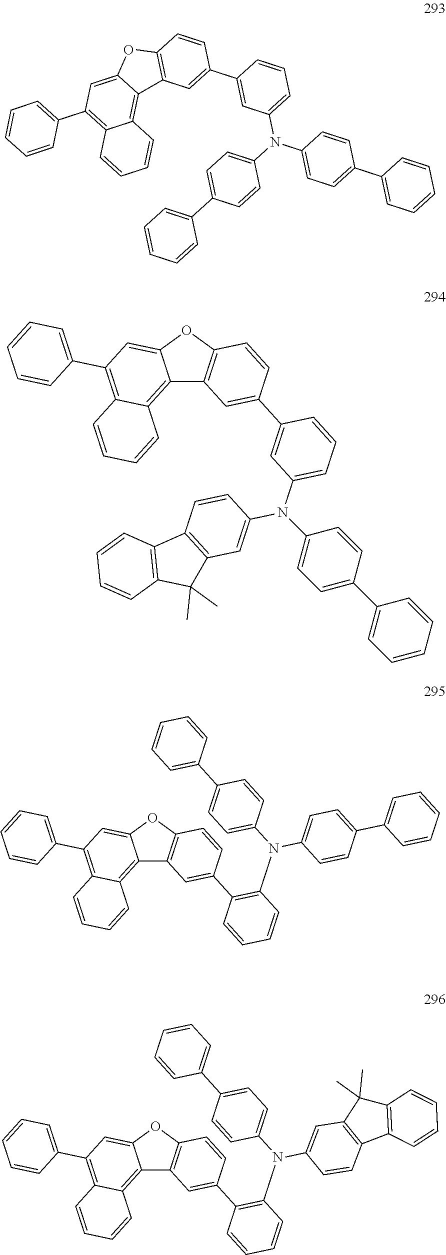 [SMILES: c1ccc(-c2ccc(N(c3ccc(-c4ccccc4)cc3)c3cccc(-c4ccc5oc6cc(-c7ccccc7)c7ccccc7c6c5c4)c3)cc2)cc1 ; CC1(C)c2ccccc2-c2ccc(N(c3ccc(-c4ccccc4)cc3)c3cccc(-c4ccc5oc6cc(-c7ccccc7)c7ccccc7c6c5c4)c3)cc21 ; c1ccc(-c2ccc(N(c3ccc(-c4ccccc4)cc3)c3ccccc3-c3ccc4oc5cc(-c6ccccc6)c6ccccc6c5c4c3)cc2)cc1 ; CC1(C)c2ccccc2-c2ccc(N(c3ccc(-c4ccccc4)cc3)c3ccccc3-c3ccc4oc5cc(-c6ccccc6)c6ccccc6c5c4c3)cc21]

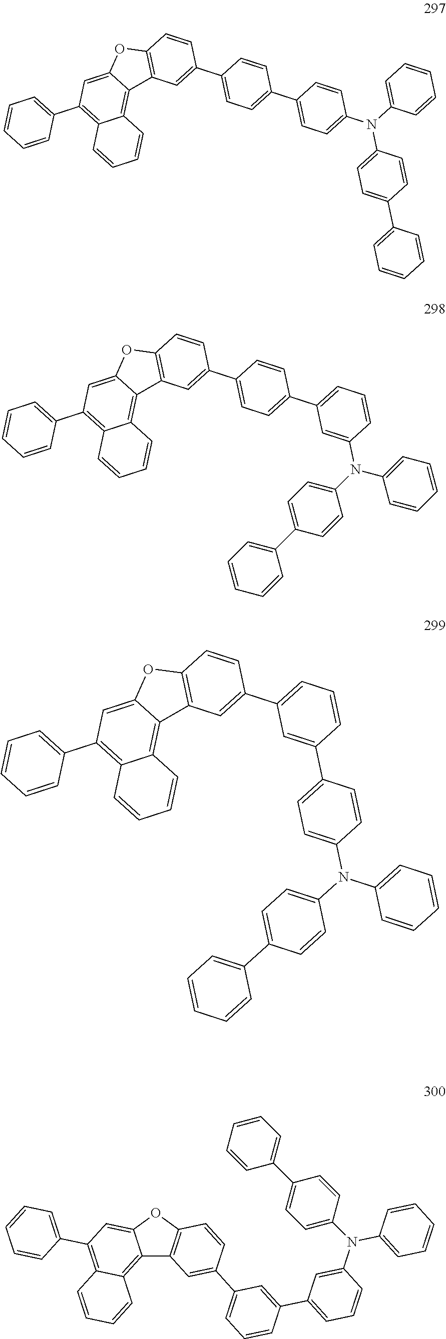 [SMILES: c1ccc(-c2ccc(N(c3ccccc3)c3ccc(-c4ccc(-c5ccc6oc7cc(-c8ccccc8)c8ccccc8c7c6c5)cc4)cc3)cc2)cc1 ; c1ccc(-c2ccc(N(c3ccccc3)c3cccc(-c4ccc(-c5ccc6oc7cc(-c8ccccc8)c8ccccc8c7c6c5)cc4)c3)cc2)cc1 ; c1ccc(-c2ccc(N(c3ccccc3)c3ccc(-c4cccc(-c5ccc6oc7cc(-c8ccccc8)c8ccccc8c7c6c5)c4)cc3)cc2)cc1 ; c1ccc(-c2ccc(N(c3ccccc3)c3cccc(-c4cccc(-c5ccc6oc7cc(-c8ccccc8)c8ccccc8c7c6c5)c4)c3)cc2)cc1]

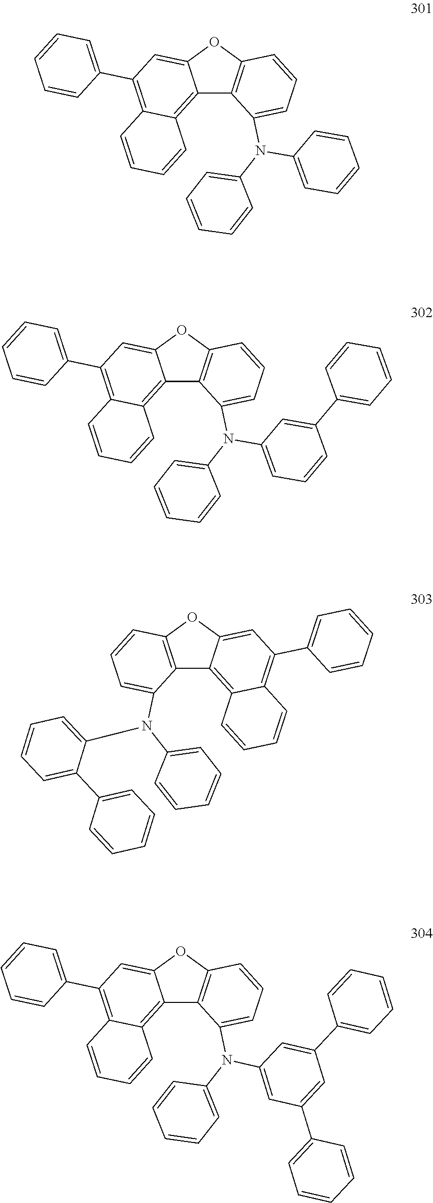 [SMILES: c1ccc(-c2cc3oc4cccc(N(c5ccccc5)c5ccccc5)c4c3c3ccccc23)cc1 ; c1ccc(-c2cccc(N(c3ccccc3)c3cccc4oc5cc(-c6ccccc6)c6ccccc6c5c34)c2)cc1 ; c1ccc(-c2ccccc2N(c2ccccc2)c2cccc3oc4cc(-c5ccccc5)c5ccccc5c4c23)cc1 ; c1ccc(-c2cc(-c3ccccc3)cc(N(c3ccccc3)c3cccc4oc5cc(-c6ccccc6)c6ccccc6c5c34)c2)cc1]

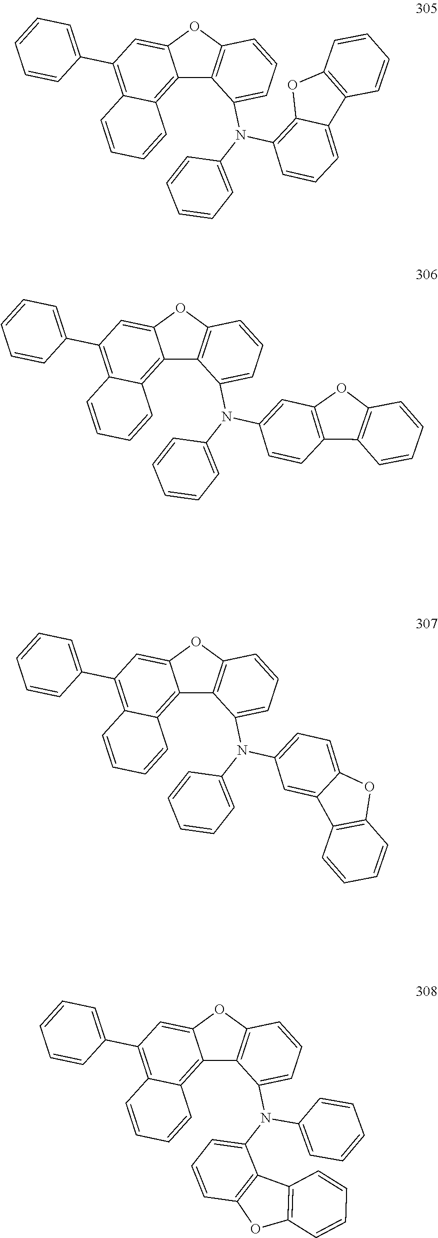 [SMILES: c1ccc(-c2cc3oc4cccc(N(c5ccccc5)c5cccc6c5oc5ccccc56)c4c3c3ccccc23)cc1 ; c1ccc(-c2cc3oc4cccc(N(c5ccccc5)c5ccc6c(c5)oc5ccccc56)c4c3c3ccccc23)cc1 ; c1ccc(-c2cc3oc4cccc(N(c5ccccc5)c5ccc6oc7ccccc7c6c5)c4c3c3ccccc23)cc1 ; c1ccc(-c2cc3oc4cccc(N(c5ccccc5)c5cccc6oc7ccccc7c56)c4c3c3ccccc23)cc1]

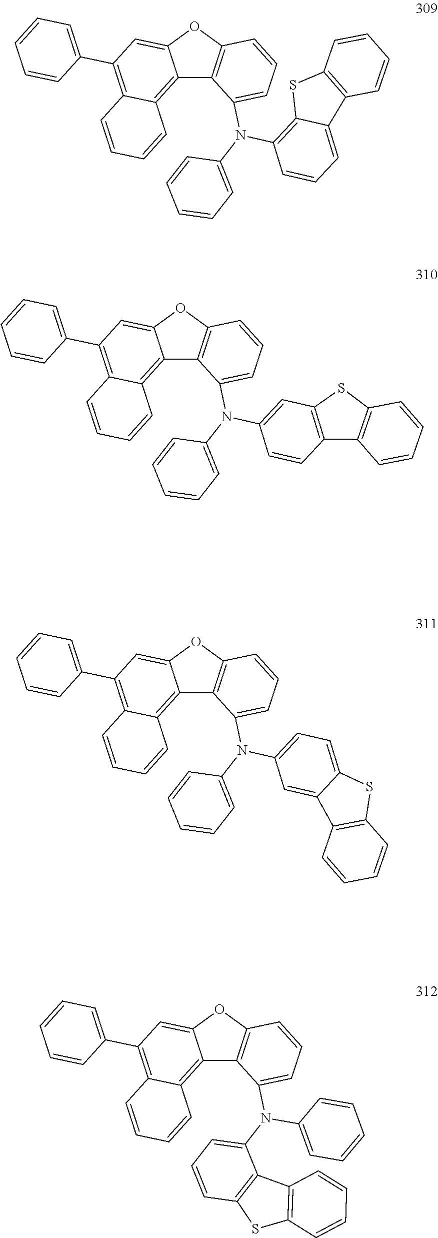 [SMILES: c1ccc(-c2cc3oc4cccc(N(c5ccccc5)c5cccc6c5sc5ccccc56)c4c3c3ccccc23)cc1 ; c1ccc(-c2cc3oc4cccc(N(c5ccccc5)c5ccc6c(c5)sc5ccccc56)c4c3c3ccccc23)cc1 ; c1ccc(-c2cc3oc4cccc(N(c5ccccc5)c5ccc6sc7ccccc7c6c5)c4c3c3ccccc23)cc1 ; c1ccc(-c2cc3oc4cccc(N(c5ccccc5)c5cccc6sc7ccccc7c56)c4c3c3ccccc23)cc1]

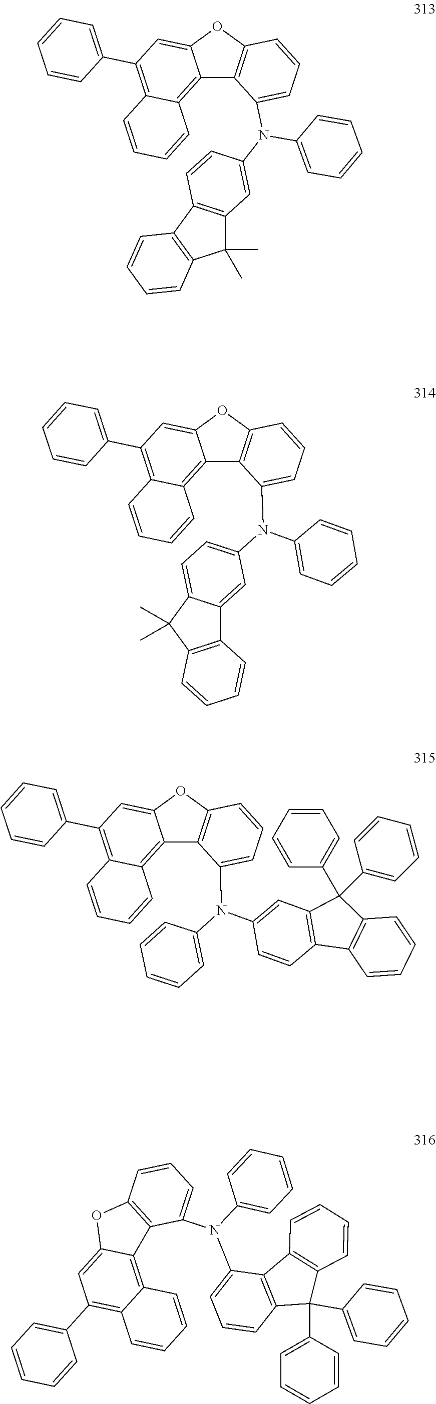

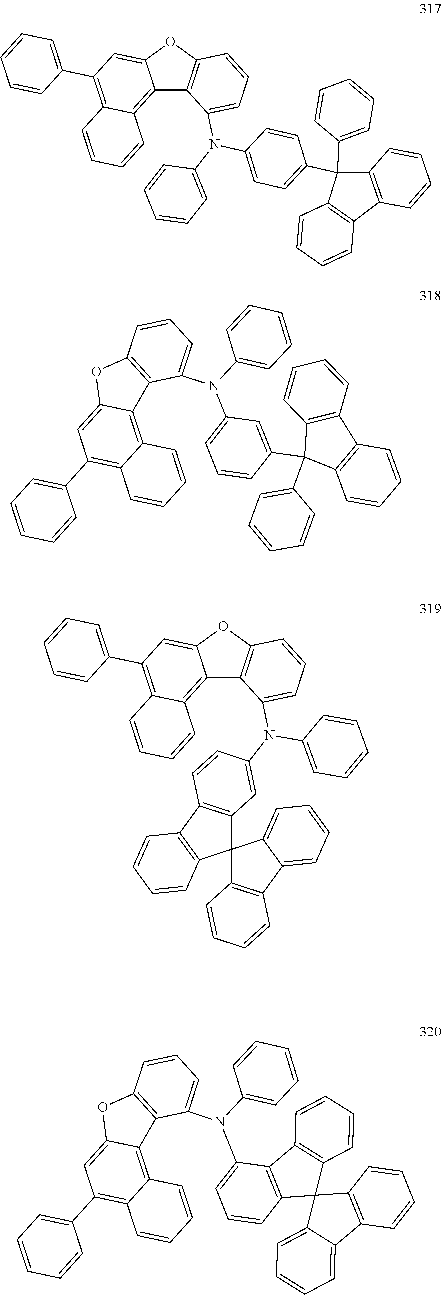 [SMILES: c1ccc(-c2cc3oc4cccc(N(c5ccccc5)c5ccc(C6(c7ccccc7)c7ccccc7-c7ccccc76)cc5)c4c3c3ccccc23)cc1 ; c1ccc(-c2cc3oc4cccc(N(c5ccccc5)c5cccc(C6(c7ccccc7)c7ccccc7-c7ccccc76)c5)c4c3c3ccccc23)cc1 ; c1ccc(-c2cc3oc4cccc(N(c5ccccc5)c5ccc6c(c5)C5(c7ccccc7-c7ccccc75)c5ccccc5-6)c4c3c3ccccc23)cc1 ; c1ccc(-c2cc3oc4cccc(N(c5ccccc5)c5cccc6c5-c5ccccc5C65c6ccccc6-c6ccccc65)c4c3c3ccccc23)cc1]

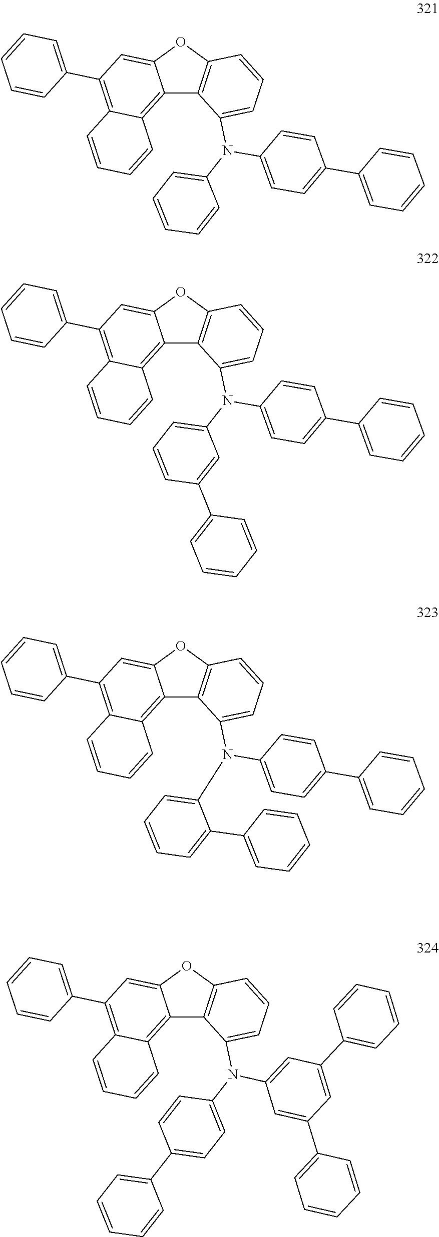 [SMILES: c1ccc(-c2ccc(N(c3ccccc3)c3cccc4oc5cc(-c6ccccc6)c6ccccc6c5c34)cc2)cc1 ; c1ccc(-c2ccc(N(c3cccc(-c4ccccc4)c3)c3cccc4oc5cc(-c6ccccc6)c6ccccc6c5c34)cc2)cc1 ; c1ccc(-c2ccc(N(c3ccccc3-c3ccccc3)c3cccc4oc5cc(-c6ccccc6)c6ccccc6c5c34)cc2)cc1 ; c1ccc(-c2ccc(N(c3cc(-c4ccccc4)cc(-c4ccccc4)c3)c3cccc4oc5cc(-c6ccccc6)c6ccccc6c5c34)cc2)cc1]

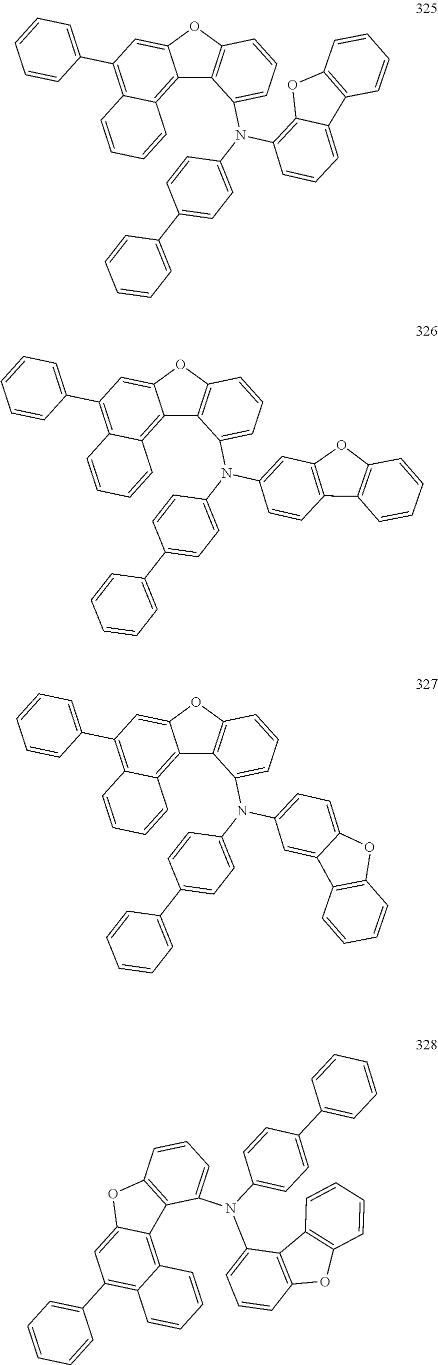 [SMILES: c1ccc(-c2ccc(N(c3cccc4c3oc3ccccc34)c3cccc4oc5cc(-c6ccccc6)c6ccccc6c5c34)cc2)cc1 ; c1ccc(-c2ccc(N(c3ccc4c(c3)oc3ccccc34)c3cccc4oc5cc(-c6ccccc6)c6ccccc6c5c34)cc2)cc1 ; c1ccc(-c2ccc(N(c3ccc4oc5ccccc5c4c3)c3cccc4oc5cc(-c6ccccc6)c6ccccc6c5c34)cc2)cc1 ; c1ccc(-c2ccc(N(c3cccc4oc5ccccc5c34)c3cccc4oc5cc(-c6ccccc6)c6ccccc6c5c34)cc2)cc1]

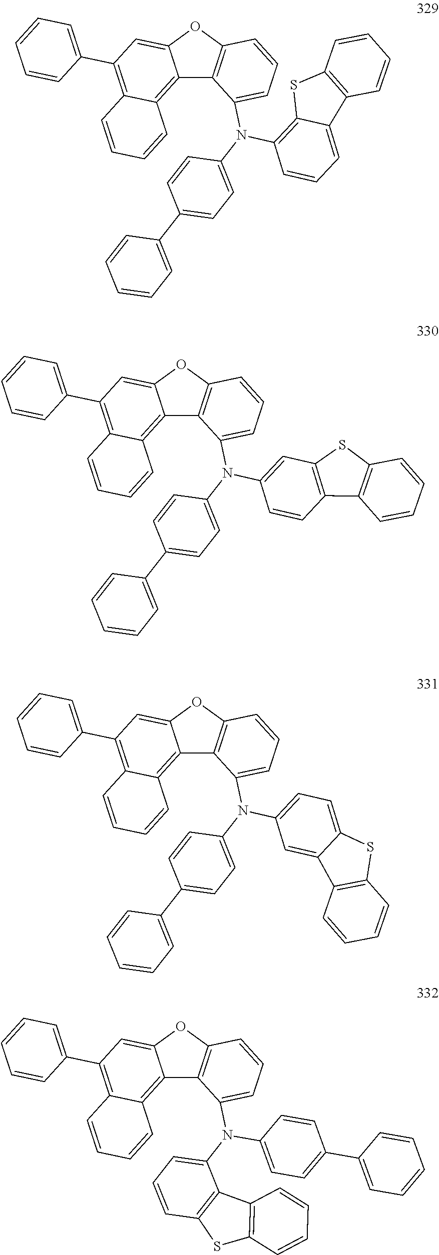 [SMILES: c1ccc(-c2ccc(N(c3cccc4c3sc3ccccc34)c3cccc4oc5cc(-c6ccccc6)c6ccccc6c5c34)cc2)cc1 ; c1ccc(-c2ccc(N(c3ccc4c(c3)sc3ccccc34)c3cccc4oc5cc(-c6ccccc6)c6ccccc6c5c34)cc2)cc1 ; c1ccc(-c2ccc(N(c3ccc4sc5ccccc5c4c3)c3cccc4oc5cc(-c6ccccc6)c6ccccc6c5c34)cc2)cc1 ; c1ccc(-c2ccc(N(c3cccc4sc5ccccc5c34)c3cccc4oc5cc(-c6ccccc6)c6ccccc6c5c34)cc2)cc1]

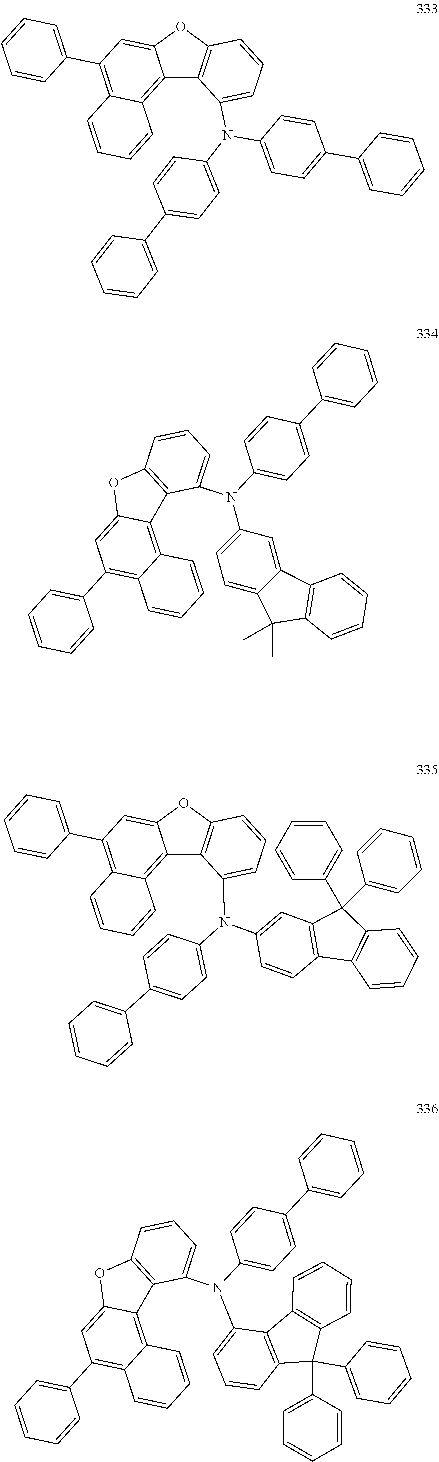

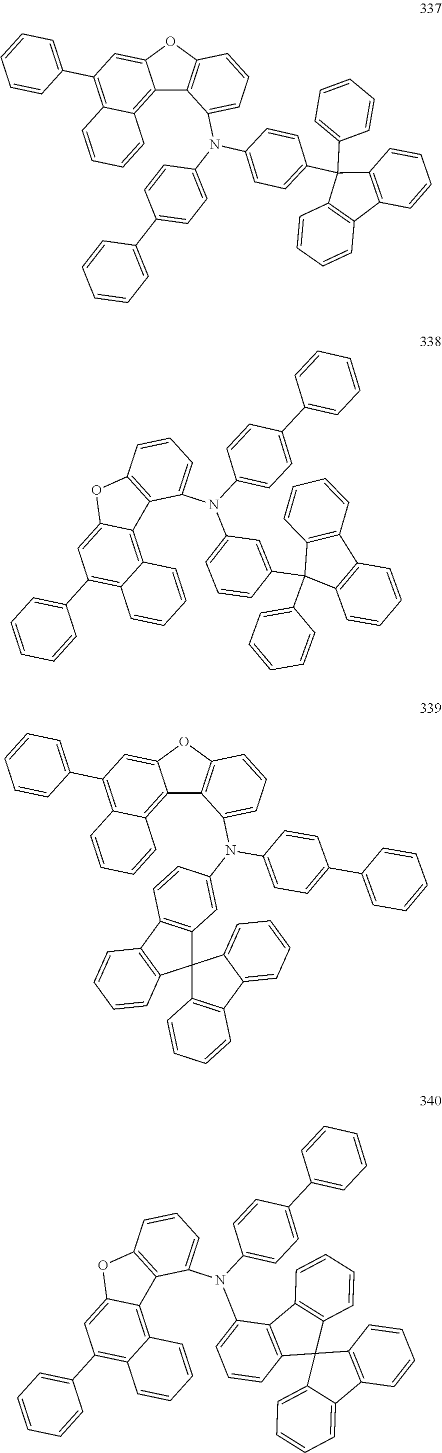 [SMILES: c1ccc(-c2ccc(N(c3ccc(C4(c5ccccc5)c5ccccc5-c5ccccc54)cc3)c3cccc4oc5cc(-c6ccccc6)c6ccccc6c5c34)cc2)cc1 ; c1ccc(-c2ccc(N(c3cccc(C4(c5ccccc5)c5ccccc5-c5ccccc54)c3)c3cccc4oc5cc(-c6ccccc6)c6ccccc6c5c34)cc2)cc1 ; c1ccc(-c2ccc(N(c3ccc4c(c3)C3(c5ccccc5-c5ccccc53)c3ccccc3-4)c3cccc4oc5cc(-c6ccccc6)c6ccccc6c5c34)cc2)cc1 ; c1ccc(-c2ccc(N(c3cccc4c3-c3ccccc3C43c4ccccc4-c4ccccc43)c3cccc4oc5cc(-c6ccccc6)c6ccccc6c5c34)cc2)cc1]

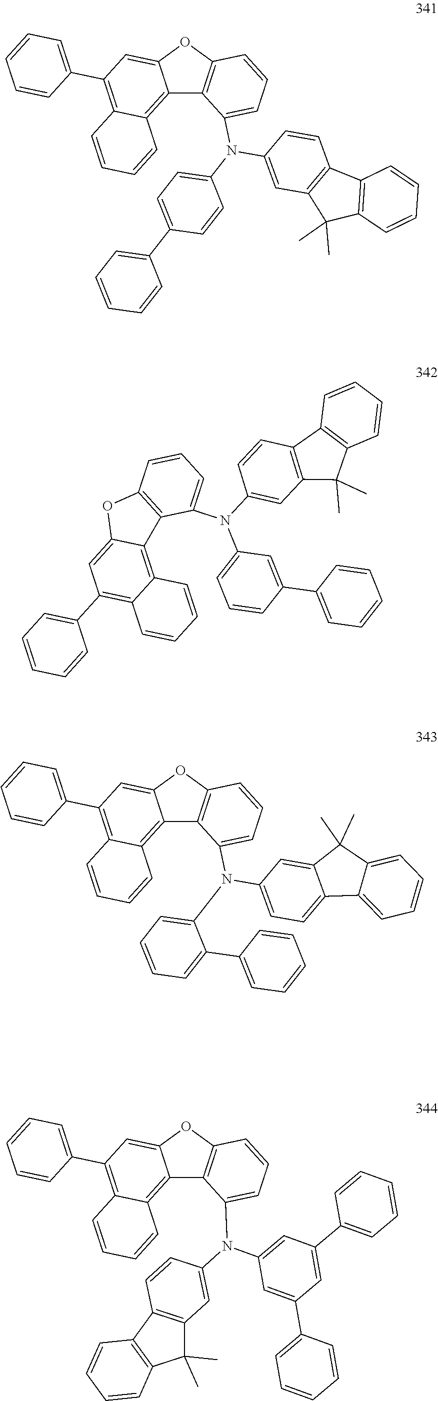 [SMILES: CC1(C)c2ccccc2-c2ccc(N(c3ccc(-c4ccccc4)cc3)c3cccc4oc5cc(-c6ccccc6)c6ccccc6c5c34)cc21 ; CC1(C)c2ccccc2-c2ccc(N(c3cccc(-c4ccccc4)c3)c3cccc4oc5cc(-c6ccccc6)c6ccccc6c5c34)cc21 ; CC1(C)c2ccccc2-c2ccc(N(c3ccccc3-c3ccccc3)c3cccc4oc5cc(-c6ccccc6)c6ccccc6c5c34)cc21 ; CC1(C)c2ccccc2-c2ccc(N(c3cc(-c4ccccc4)cc(-c4ccccc4)c3)c3cccc4oc5cc(-c6ccccc6)c6ccccc6c5c34)cc21]

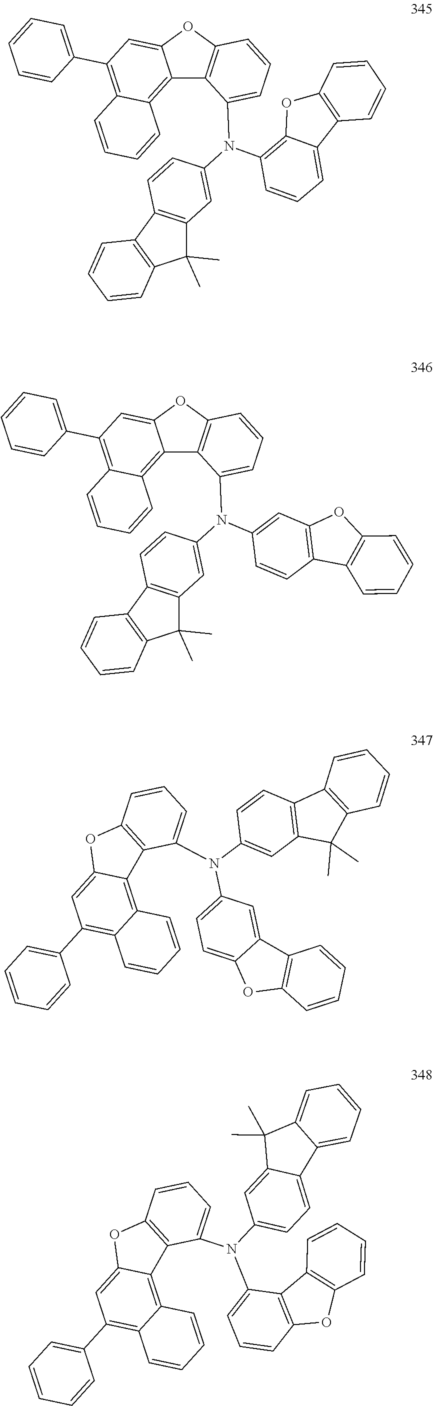 [SMILES: CC1(C)c2ccccc2-c2ccc(N(c3cccc4c3oc3ccccc34)c3cccc4oc5cc(-c6ccccc6)c6ccccc6c5c34)cc21 ; CC1(C)c2ccccc2-c2ccc(N(c3ccc4c(c3)oc3ccccc34)c3cccc4oc5cc(-c6ccccc6)c6ccccc6c5c34)cc21 ; CC1(C)c2ccccc2-c2ccc(N(c3ccc4oc5ccccc5c4c3)c3cccc4oc5cc(-c6ccccc6)c6ccccc6c5c34)cc21 ; CC1(C)c2ccccc2-c2ccc(N(c3cccc4oc5ccccc5c34)c3cccc4oc5cc(-c6ccccc6)c6ccccc6c5c34)cc21]

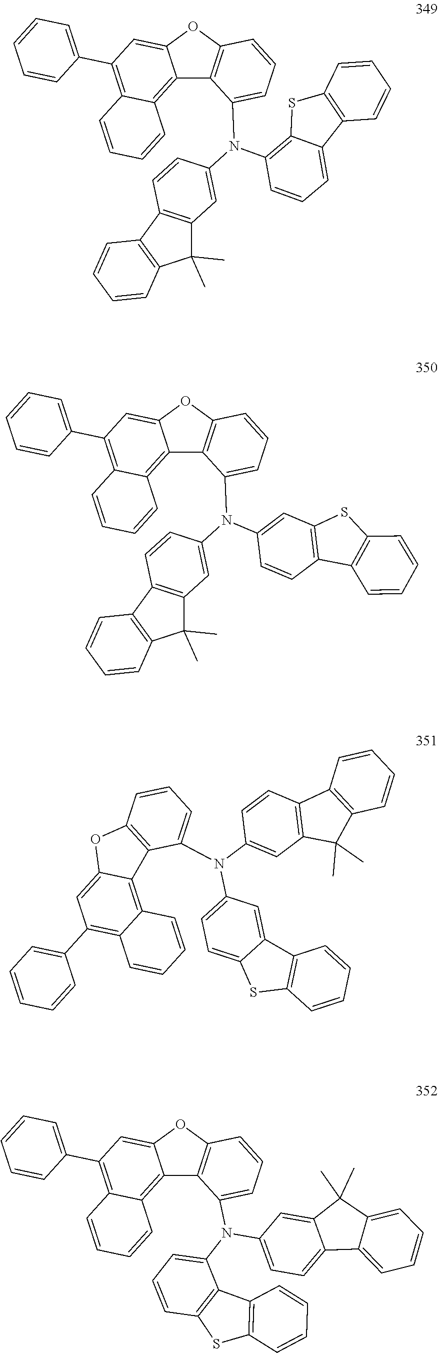 [SMILES: CC1(C)c2ccccc2-c2ccc(N(c3cccc4c3sc3ccccc34)c3cccc4oc5cc(-c6ccccc6)c6ccccc6c5c34)cc21 ; CC1(C)c2ccccc2-c2ccc(N(c3ccc4c(c3)sc3ccccc34)c3cccc4oc5cc(-c6ccccc6)c6ccccc6c5c34)cc21 ; CC1(C)c2ccccc2-c2ccc(N(c3ccc4sc5ccccc5c4c3)c3cccc4oc5cc(-c6ccccc6)c6ccccc6c5c34)cc21 ; CC1(C)c2ccccc2-c2ccc(N(c3cccc4sc5ccccc5c34)c3cccc4oc5cc(-c6ccccc6)c6ccccc6c5c34)cc21]

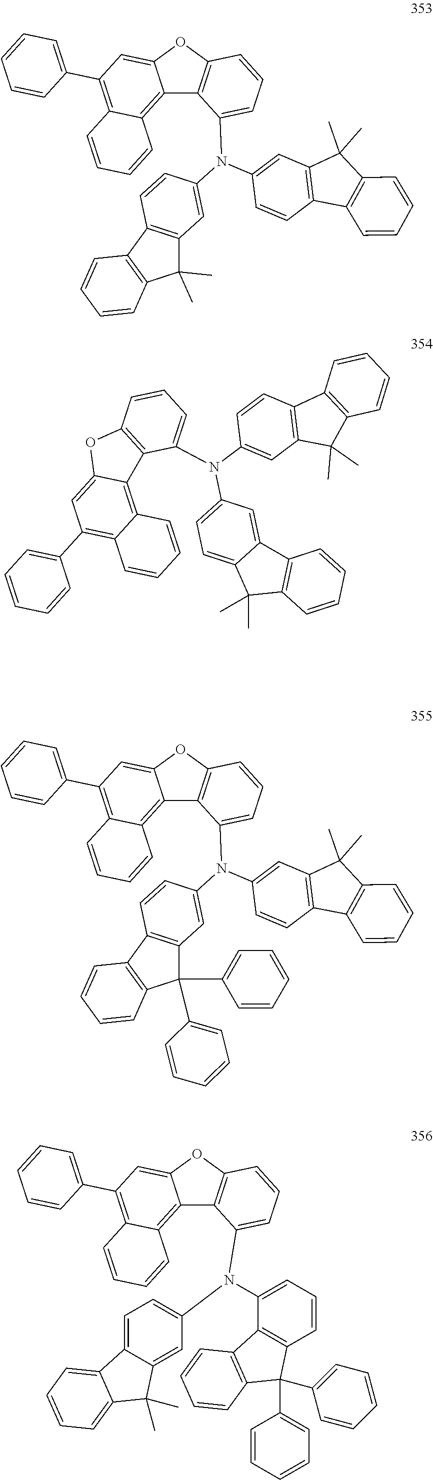 [SMILES: CC1(C)c2ccccc2-c2ccc(N(c3ccc4c(c3)C(C)(C)c3ccccc3-4)c3cccc4oc5cc(-c6ccccc6)c6ccccc6c5c34)cc21 ; CC1(C)c2ccccc2-c2cc(N(c3ccc4c(c3)C(C)(C)c3ccccc3-4)c3cccc4oc5cc(-c6ccccc6)c6ccccc6c5c34)ccc21 ; CC1(C)c2ccccc2-c2ccc(N(c3ccc4c(c3)C(c3ccccc3)(c3ccccc3)c3ccccc3-4)c3cccc4oc5cc(-c6ccccc6)c6ccccc6c5c34)cc21 ; CC1(C)c2ccccc2-c2ccc(N(c3cccc4c3-c3ccccc3C4(c3ccccc3)c3ccccc3)c3cccc4oc5cc(-c6ccccc6)c6ccccc6c5c34)cc21]

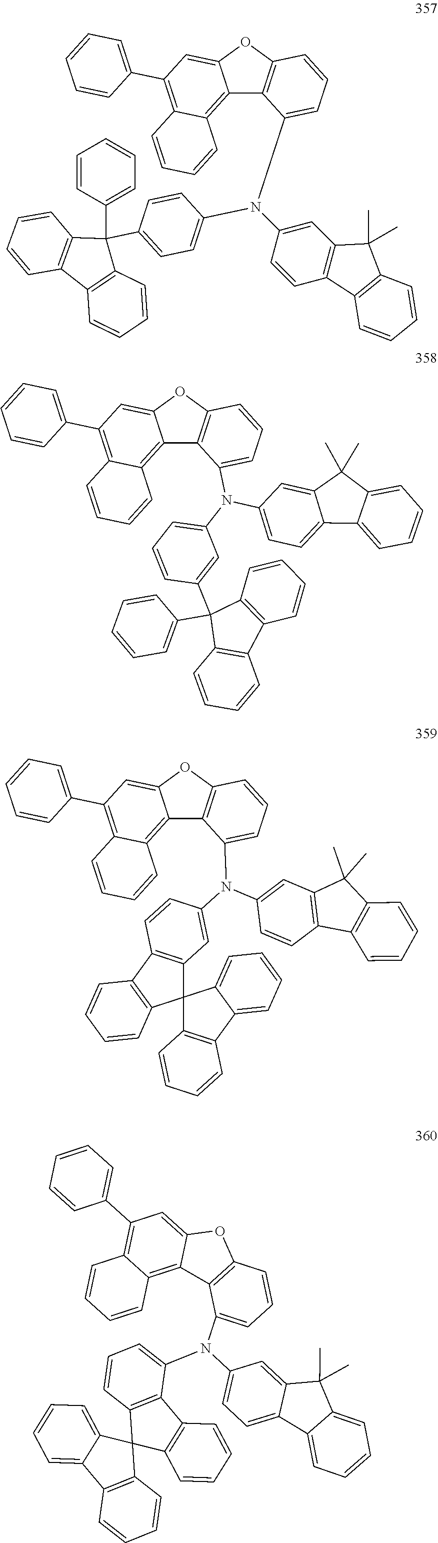 [SMILES: CC1(C)c2ccccc2-c2ccc(N(c3ccc(C4(c5ccccc5)c5ccccc5-c5ccccc54)cc3)c3cccc4oc5cc(-c6ccccc6)c6ccccc6c5c34)cc21 ; CC1(C)c2ccccc2-c2ccc(N(c3cccc(C4(c5ccccc5)c5ccccc5-c5ccccc54)c3)c3cccc4oc5cc(-c6ccccc6)c6ccccc6c5c34)cc21 ; CC1(C)c2ccccc2-c2ccc(N(c3ccc4c(c3)C3(c5ccccc5-c5ccccc53)c3ccccc3-4)c3cccc4oc5cc(-c6ccccc6)c6ccccc6c5c34)cc21 ; CC1(C)c2ccccc2-c2ccc(N(c3cccc4c3-c3ccccc3C43c4ccccc4-c4ccccc43)c3cccc4oc5cc(-c6ccccc6)c6ccccc6c5c34)cc21]

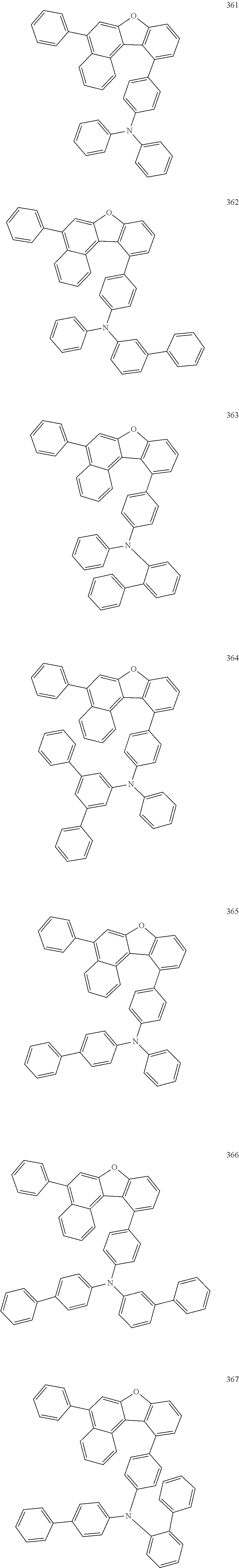

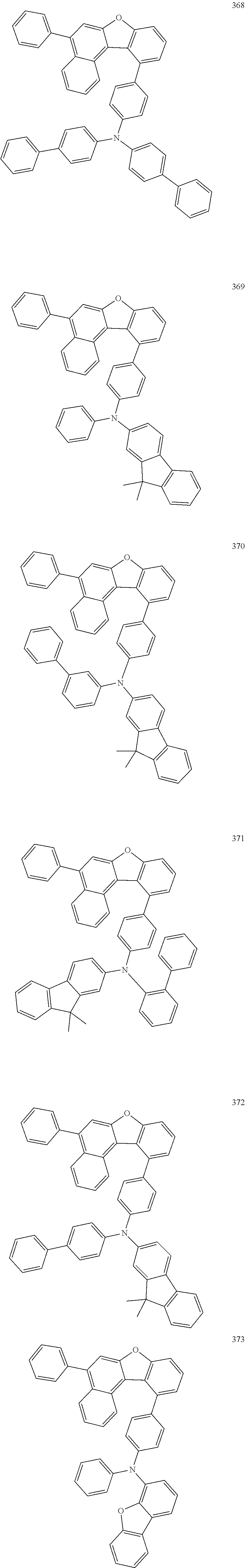

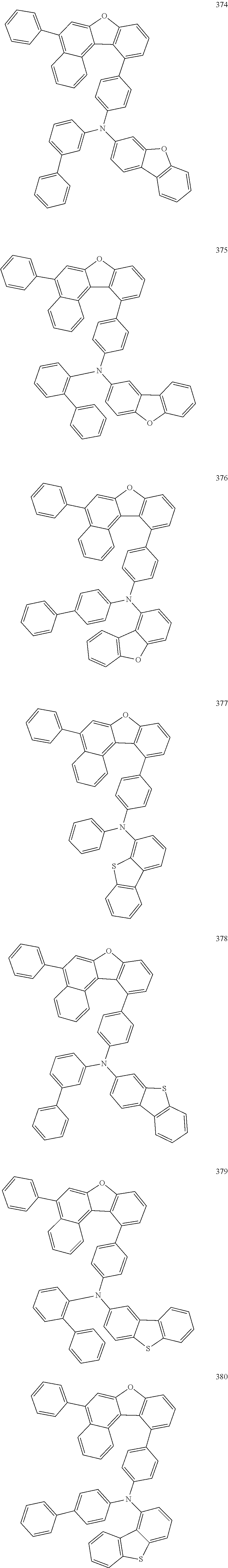

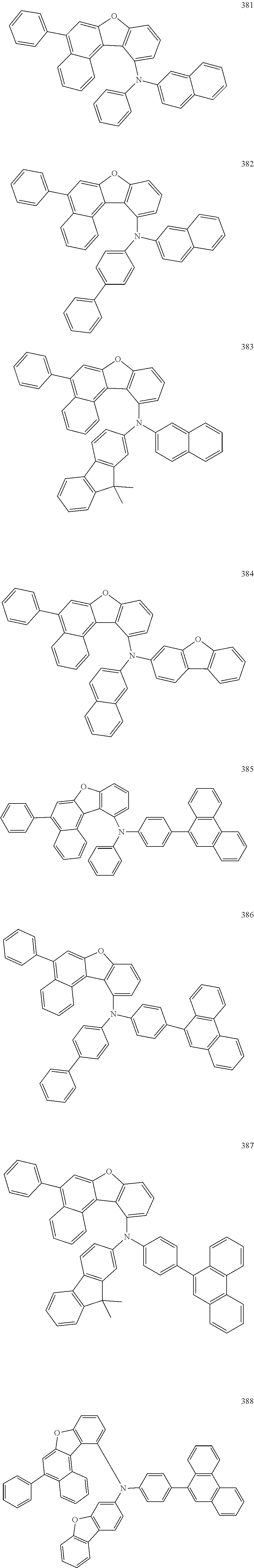

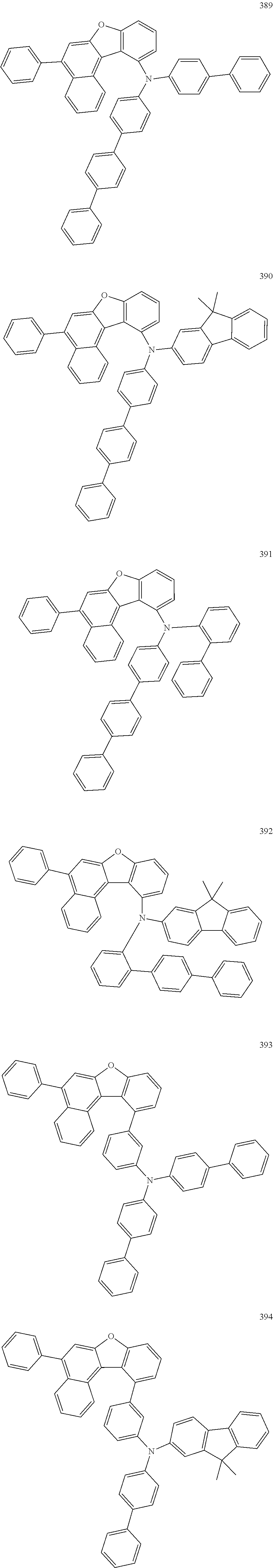

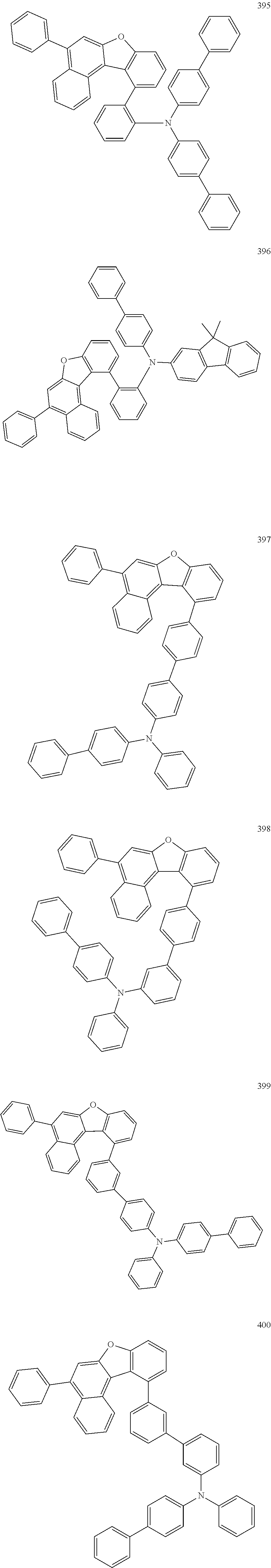

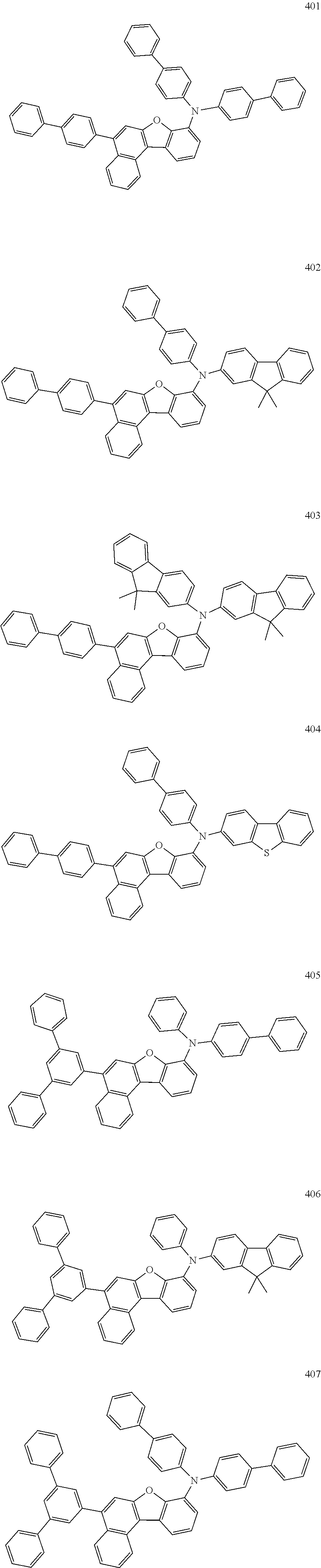

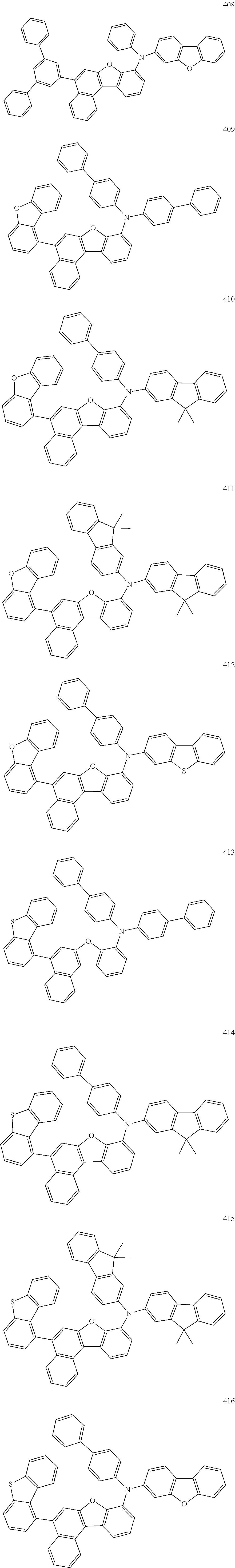

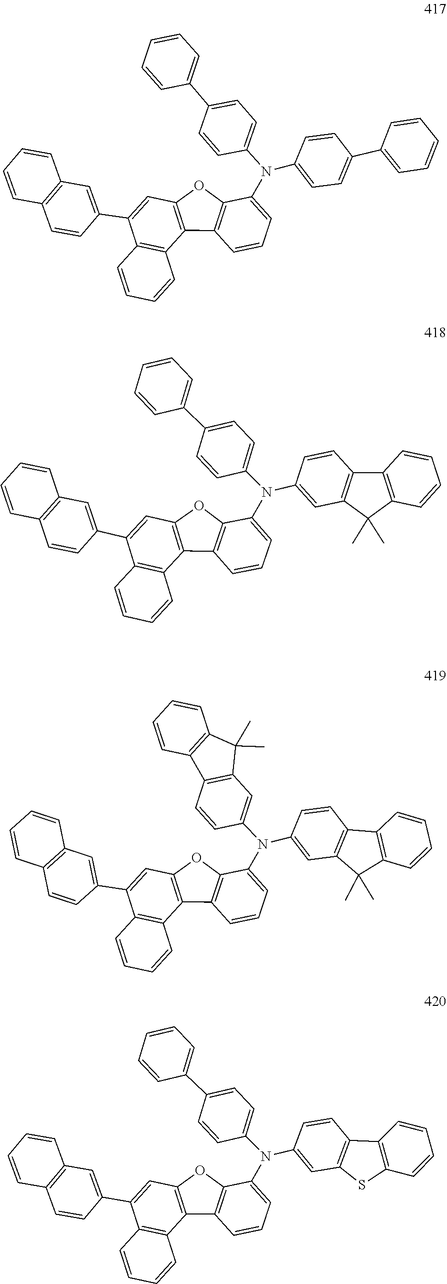 [SMILES: c1ccc(-c2ccc(N(c3ccc(-c4ccccc4)cc3)c3cccc4c3oc3cc(-c5ccc6ccccc6c5)c5ccccc5c34)cc2)cc1 ; CC1(C)c2ccccc2-c2ccc(N(c3ccc(-c4ccccc4)cc3)c3cccc4c3oc3cc(-c5ccc6ccccc6c5)c5ccccc5c34)cc21 ; CC1(C)c2ccccc2-c2ccc(N(c3ccc4c(c3)C(C)(C)c3ccccc3-4)c3cccc4c3oc3cc(-c5ccc6ccccc6c5)c5ccccc5c34)cc21 ; c1ccc(-c2ccc(N(c3ccc4c(c3)sc3ccccc34)c3cccc4c3oc3cc(-c5ccc6ccccc6c5)c5ccccc5c34)cc2)cc1]

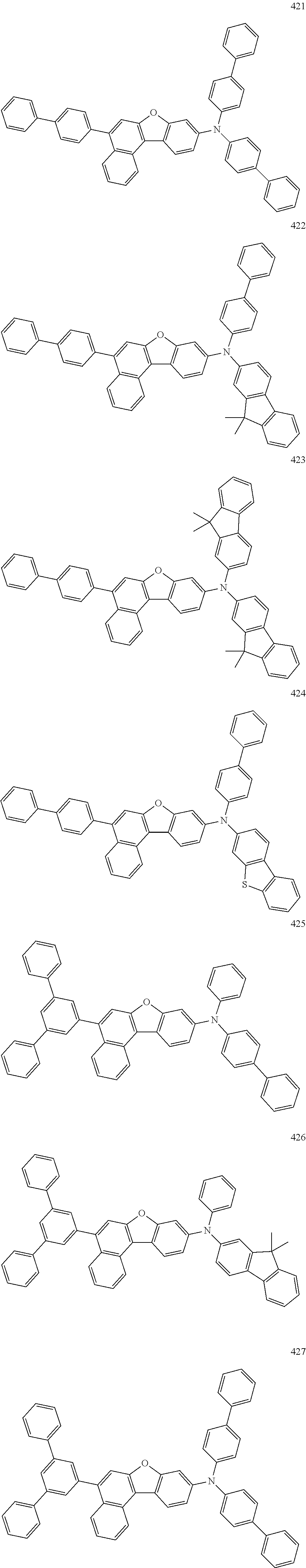

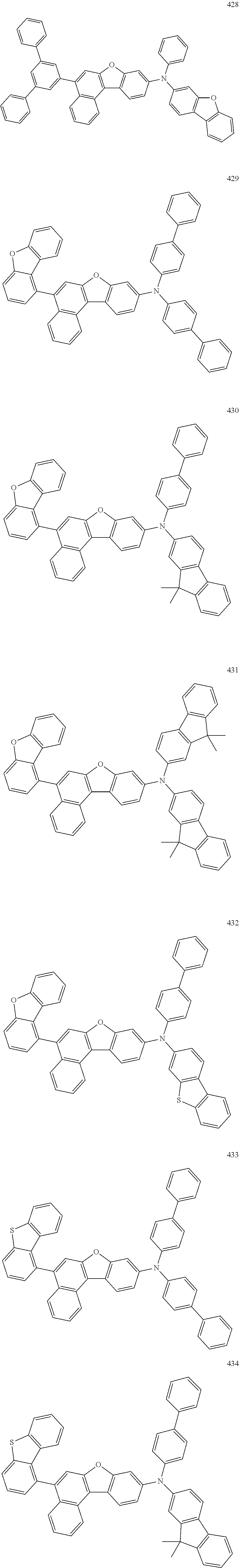

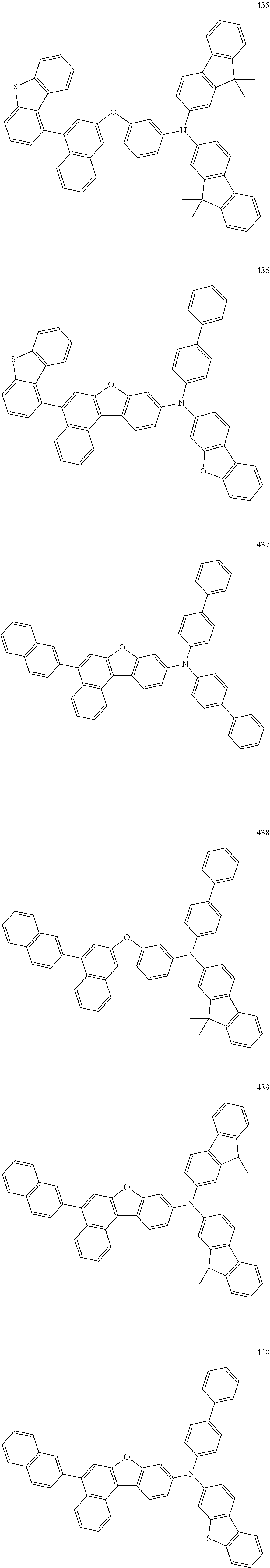

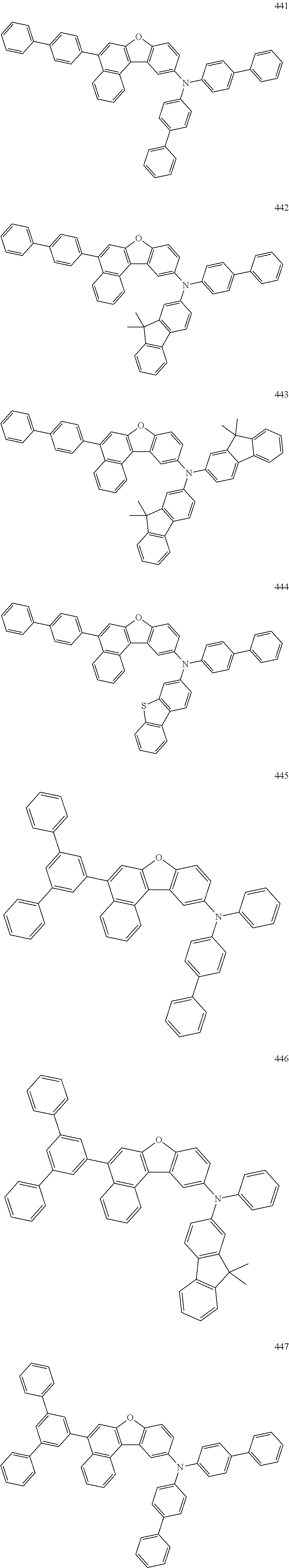

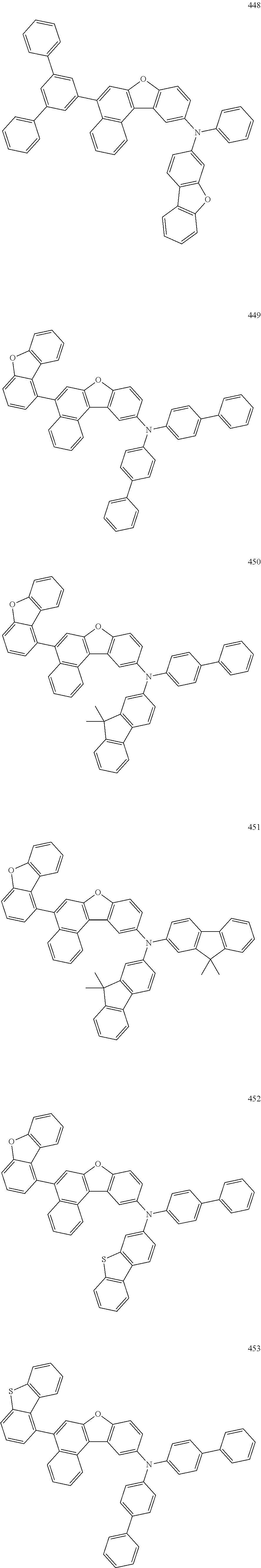

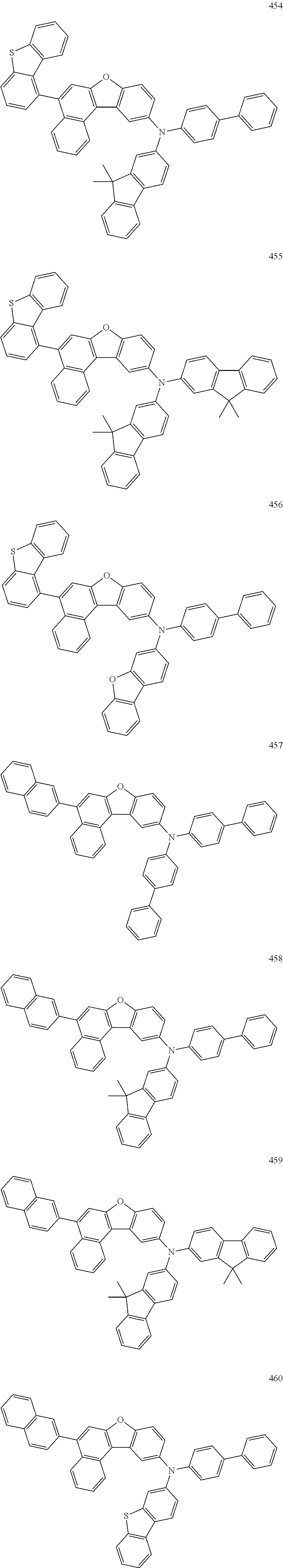

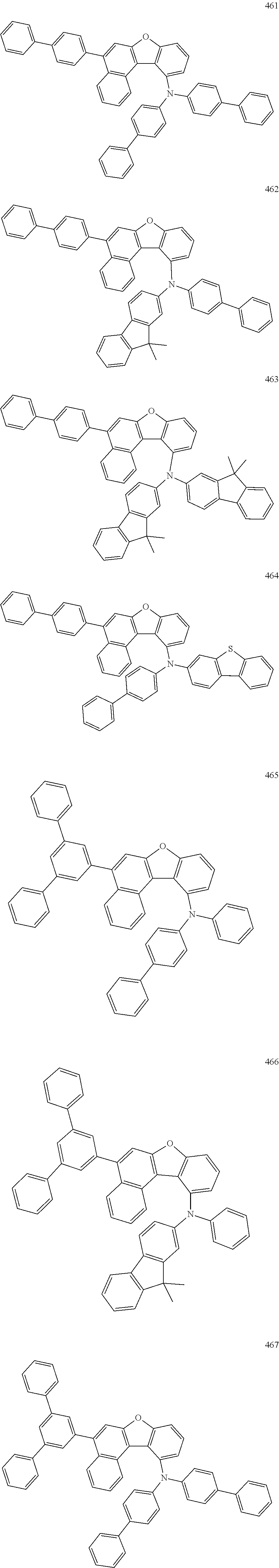

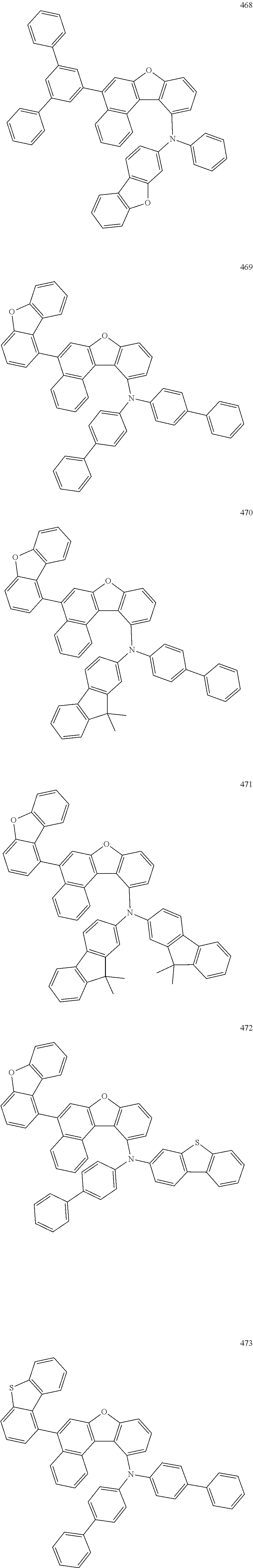

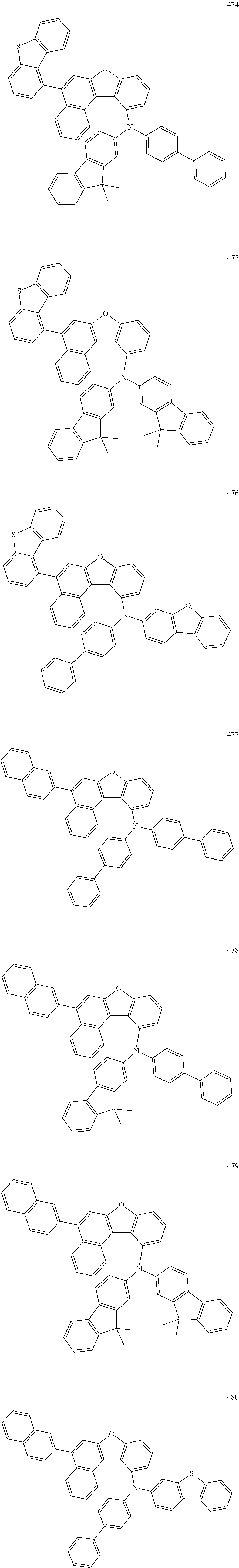

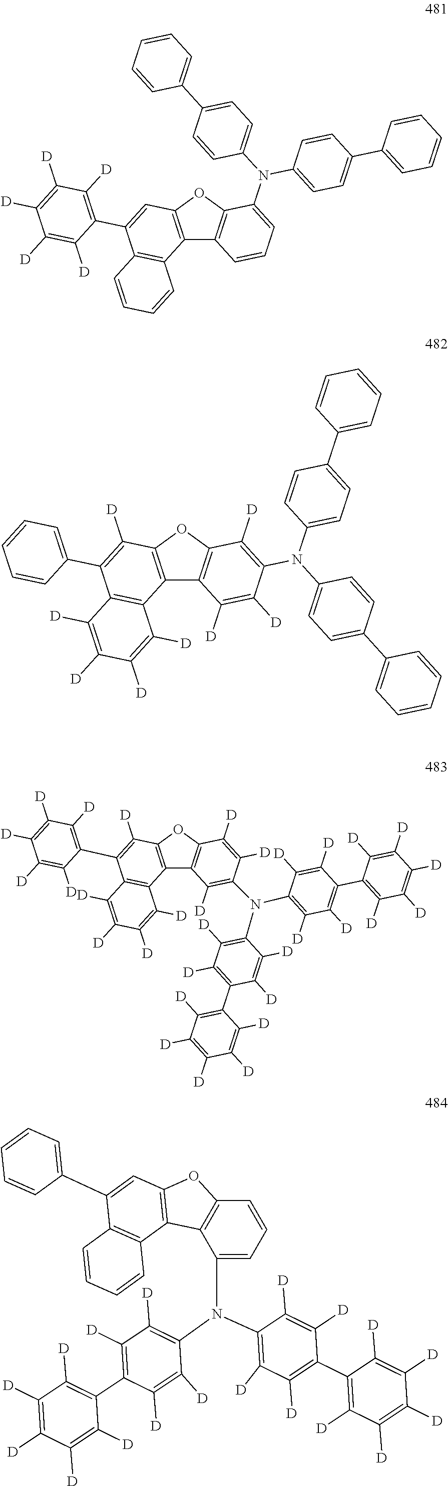

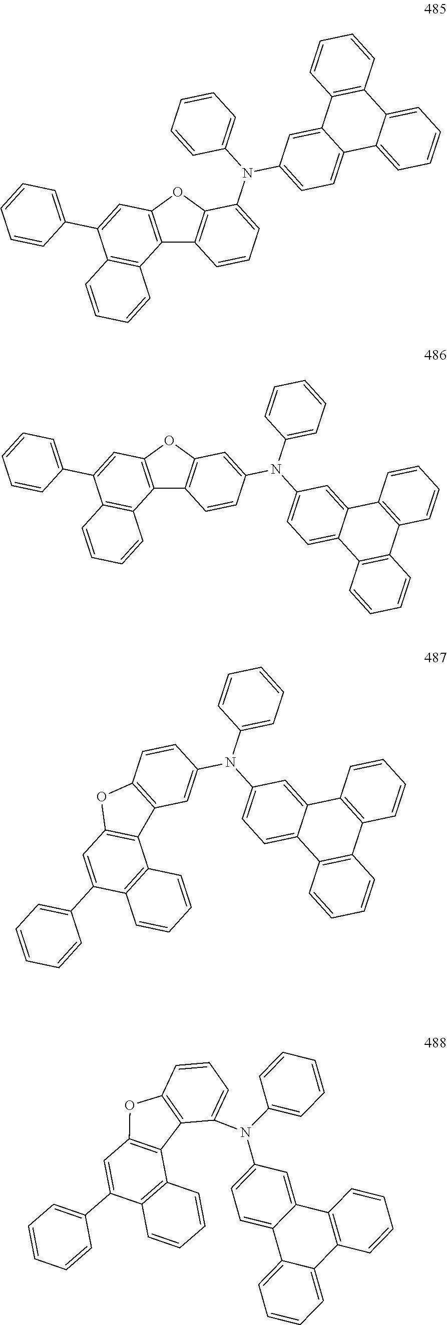 [SMILES: c1ccc(-c2cc3oc4c(N(c5ccccc5)c5ccc6c7ccccc7c7ccccc7c6c5)cccc4c3c3ccccc23)cc1 ; c1ccc(-c2cc3oc4cc(N(c5ccccc5)c5ccc6c7ccccc7c7ccccc7c6c5)ccc4c3c3ccccc23)cc1 ; c1ccc(-c2cc3oc4ccc(N(c5ccccc5)c5ccc6c7ccccc7c7ccccc7c6c5)cc4c3c3ccccc23)cc1 ; c1ccc(-c2cc3oc4cccc(N(c5ccccc5)c5ccc6c7ccccc7c7ccccc7c6c5)c4c3c3ccccc23)cc1]

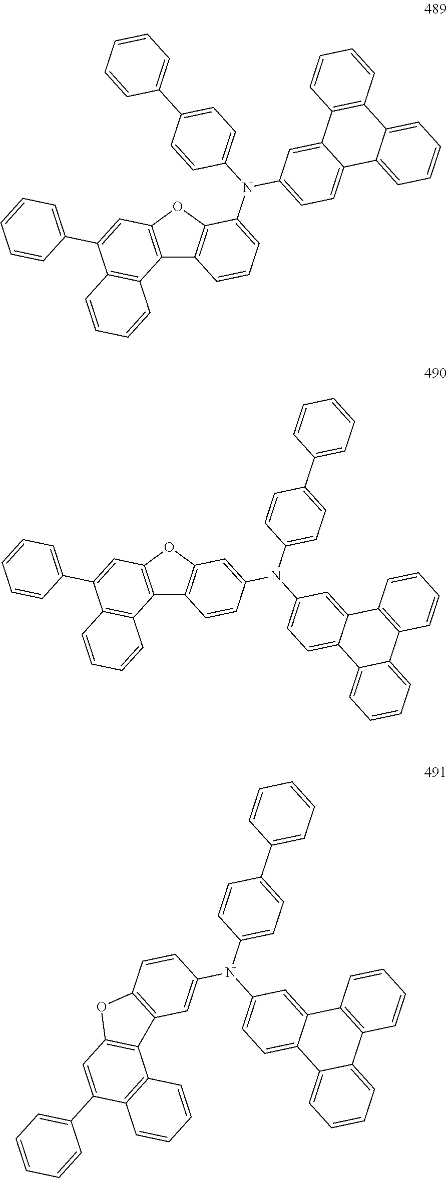 [SMILES: c1ccc(-c2ccc(N(c3ccc4c5ccccc5c5ccccc5c4c3)c3cccc4c3oc3cc(-c5ccccc5)c5ccccc5c34)cc2)cc1 ; c1ccc(-c2ccc(N(c3ccc4c(c3)oc3cc(-c5ccccc5)c5ccccc5c34)c3ccc4c5ccccc5c5ccccc5c4c3)cc2)cc1 ; c1ccc(-c2ccc(N(c3ccc4c5ccccc5c5ccccc5c4c3)c3ccc4oc5cc(-c6ccccc6)c6ccccc6c5c4c3)cc2)cc1]

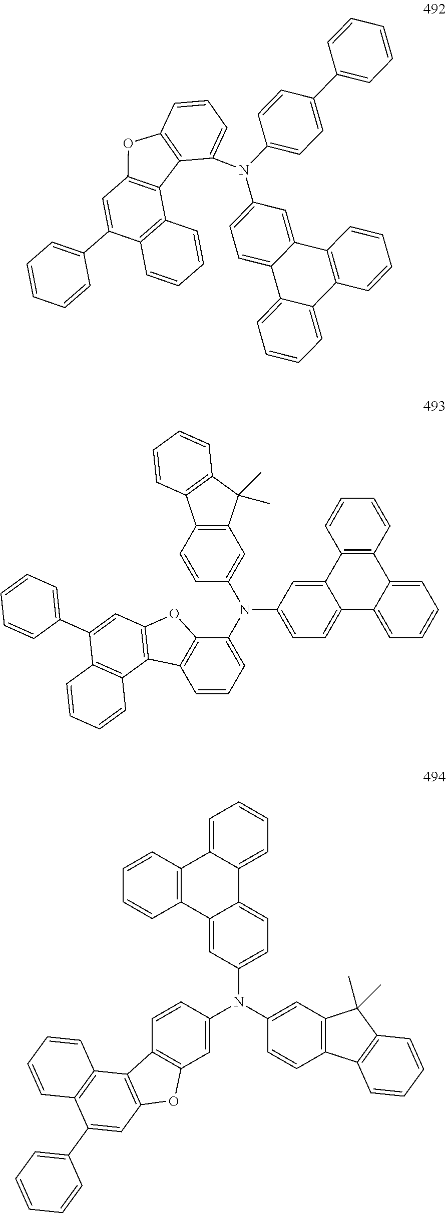 [SMILES: c1ccc(-c2ccc(N(c3ccc4c5ccccc5c5ccccc5c4c3)c3cccc4oc5cc(-c6ccccc6)c6ccccc6c5c34)cc2)cc1 ; CC1(C)c2ccccc2-c2ccc(N(c3ccc4c5ccccc5c5ccccc5c4c3)c3cccc4c3oc3cc(-c5ccccc5)c5ccccc5c34)cc21 ; CC1(C)c2ccccc2-c2ccc(N(c3ccc4c(c3)oc3cc(-c5ccccc5)c5ccccc5c34)c3ccc4c5ccccc5c5ccccc5c4c3)cc21]

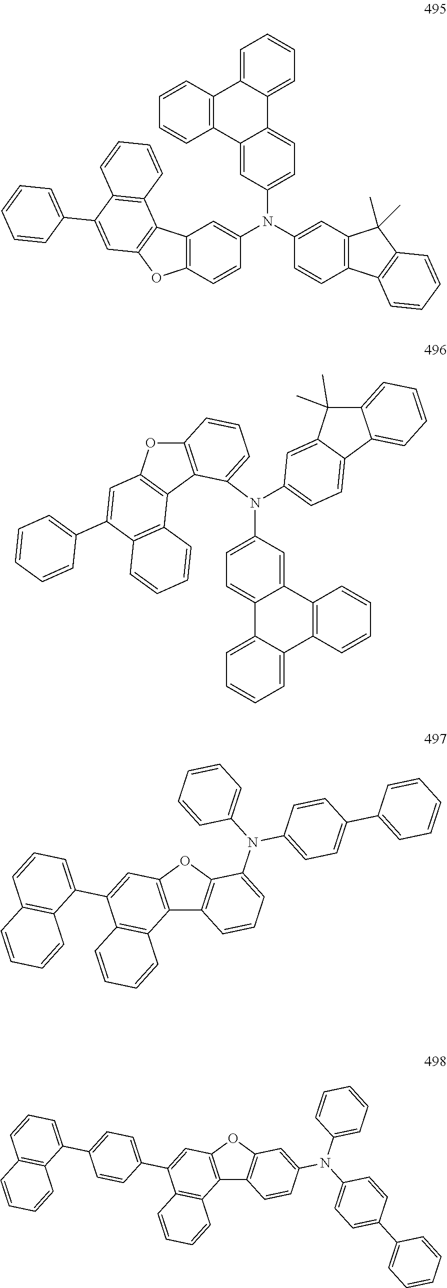 [SMILES: CC1(C)c2ccccc2-c2ccc(N(c3ccc4c5ccccc5c5ccccc5c4c3)c3ccc4oc5cc(-c6ccccc6)c6ccccc6c5c4c3)cc21 ; CC1(C)c2ccccc2-c2ccc(N(c3ccc4c5ccccc5c5ccccc5c4c3)c3cccc4oc5cc(-c6ccccc6)c6ccccc6c5c34)cc21 ; c1ccc(-c2ccc(N(c3ccccc3)c3cccc4c3oc3cc(-c5cccc6ccccc56)c5ccccc5c34)cc2)cc1 ; c1ccc(-c2ccc(N(c3ccccc3)c3ccc4c(c3)oc3cc(-c5ccc(-c6cccc7ccccc67)cc5)c5ccccc5c34)cc2)cc1]

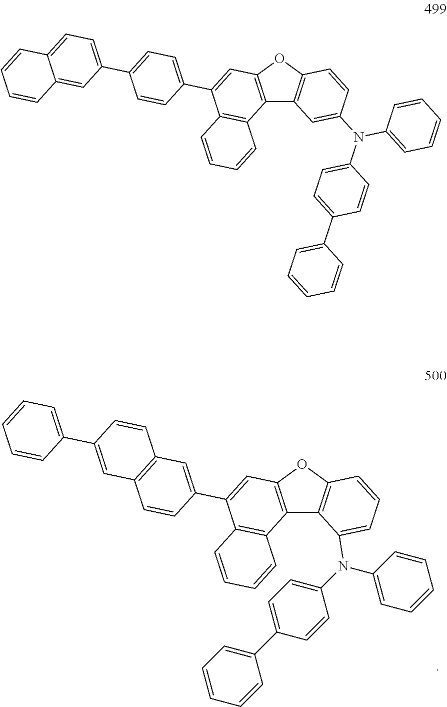 [SMILES: c1ccc(-c2ccc(N(c3ccccc3)c3ccc4oc5cc(-c6ccc(-c7ccc8ccccc8c7)cc6)c6ccccc6c5c4c3)cc2)cc1 ; c1ccc(-c2ccc(N(c3ccccc3)c3cccc4oc5cc(-c6ccc7cc(-c8ccccc8)ccc7c6)c6ccccc6c5c34)cc2)cc1]

7. An organic light emitting device comprising:
a first electrode;
a second electrode; and
an organic material layer having one or more layers provided between the first electrode and the second electrode,
wherein one or more layers of the organic material layer comprise one or more of the heterocyclic compounds of claim 1.

8. The organic light emitting device of claim 7, wherein the organic material layer comprises a hole transport layer, and the hole transport layer comprises one or more of the heterocyclic compounds.

9. The organic light emitting device of claim 7, wherein the organic material layer comprises an electron blocking layer, and the electron blocking layer comprises one or more of the heterocyclic compounds.

10. The organic light emitting device of claim 7, wherein the organic material layer comprises a light emitting layer, and the light emitting layer comprises one or more of the heterocyclic compounds.

11. The organic light emitting device of claim 7, wherein the organic material layer comprises a light emitting layer, the light emitting layer comprises a host, and the host comprises one or more of the heterocyclic compounds.

12. The organic light emitting device of claim 10, wherein the light emitting layer further comprises a compound of the following Chemical Formula 2: 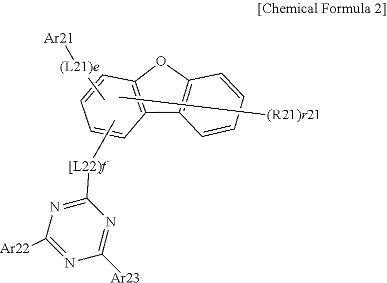
in Chemical Formula 2,
L21 and L22 are each independently a direct bond; or a substituted or unsubstituted C6 to C60 arylene group,
e and f are an integer from 0 to 4,
when e and f are 2 or higher, substituents in the parenthesis are the same as or different from each other,
Ar21 is a substituted or unsubstituted C6 to C60 aryl group,
Ar22 and Ar23 are each independently a substituted or unsubstituted C6 to C60 aryl group; or a substituted or unsubstituted C2 to C60 heteroaryl group,
R21 is hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, and
r21 is an integer from 0 to 6, and when r21 is 2 or higher, R21's are the same as or different from each other.

13. The organic light emitting device of claim 12, wherein Chemical Formula 2 is represented by any one of the following compounds:

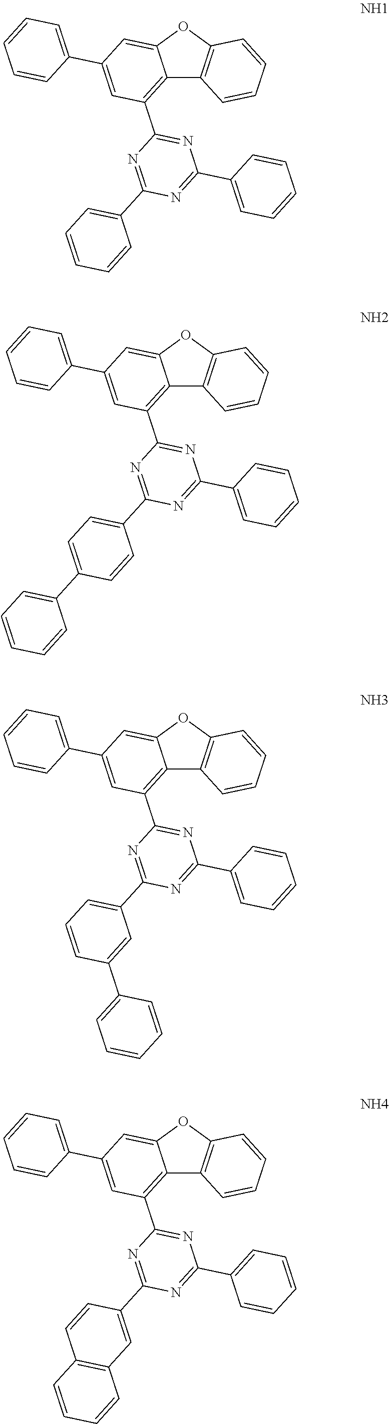 [SMILES: c1ccc(-c2cc(-c3nc(-c4ccccc4)nc(-c4ccccc4)n3)c3c(c2)oc2ccccc23)cc1 ; c1ccc(-c2ccc(-c3nc(-c4ccccc4)nc(-c4cc(-c5ccccc5)cc5oc6ccccc6c45)n3)cc2)cc1 ; c1ccc(-c2cccc(-c3nc(-c4ccccc4)nc(-c4cc(-c5ccccc5)cc5oc6ccccc6c45)n3)c2)cc1 ; c1ccc(-c2cc(-c3nc(-c4ccccc4)nc(-c4ccc5ccccc5c4)n3)c3c(c2)oc2ccccc23)cc1]

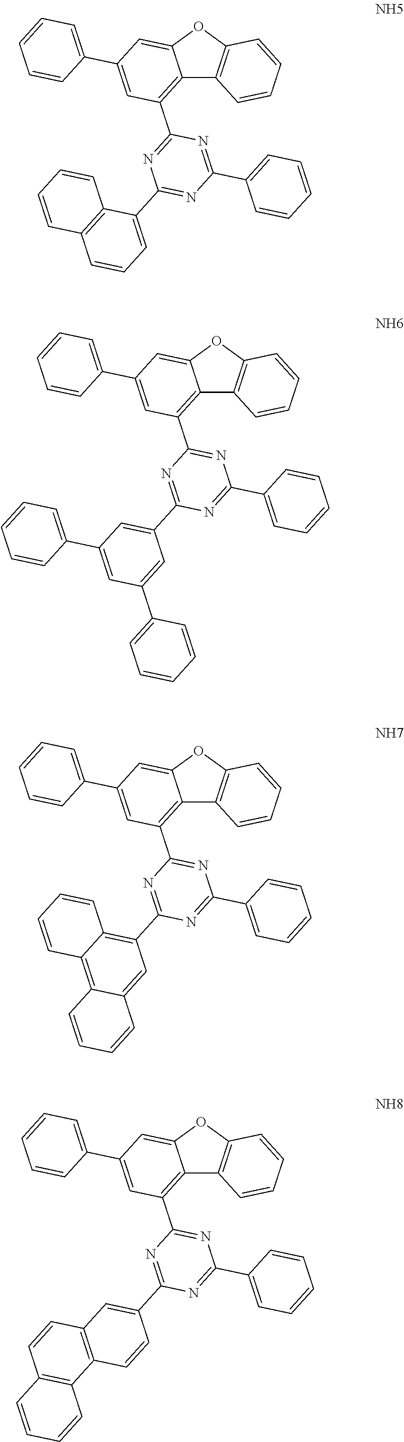 [SMILES: c1ccc(-c2cc(-c3nc(-c4ccccc4)nc(-c4cccc5ccccc45)n3)c3c(c2)oc2ccccc23)cc1 ; c1ccc(-c2cc(-c3ccccc3)cc(-c3nc(-c4ccccc4)nc(-c4cc(-c5ccccc5)cc5oc6ccccc6c45)n3)c2)cc1 ; c1ccc(-c2cc(-c3nc(-c4ccccc4)nc(-c4cc5ccccc5c5ccccc45)n3)c3c(c2)oc2ccccc23)cc1 ; c1ccc(-c2cc(-c3nc(-c4ccccc4)nc(-c4ccc5c(ccc6ccccc65)c4)n3)c3c(c2)oc2ccccc23)cc1]

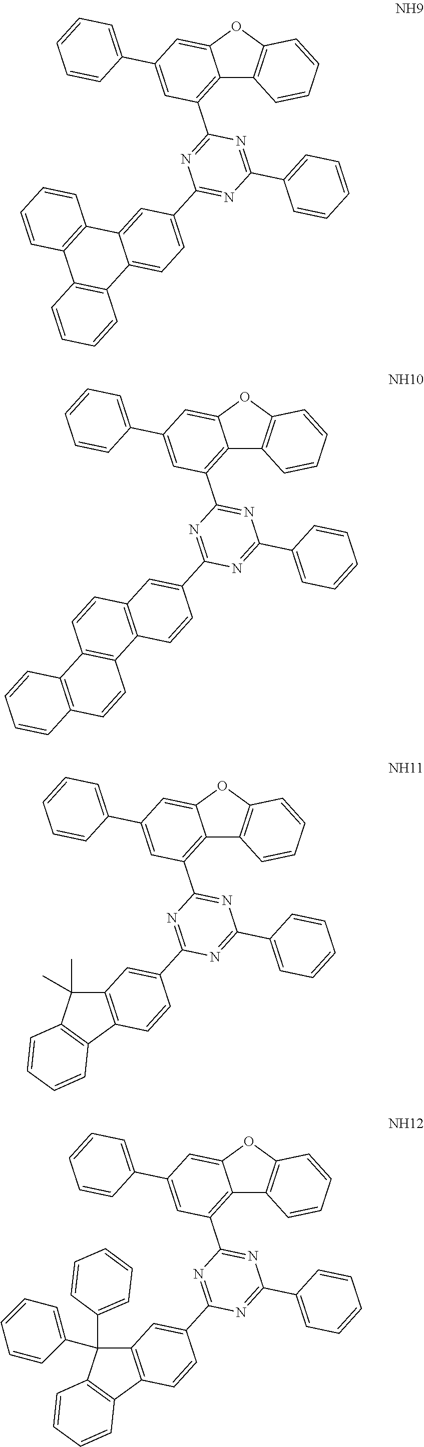

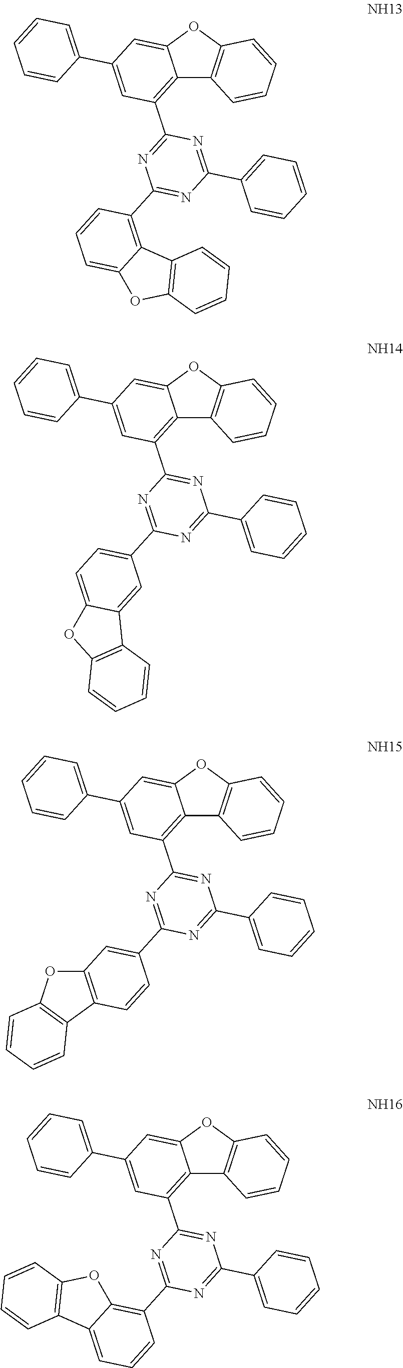 [SMILES: c1ccc(-c2cc(-c3nc(-c4ccccc4)nc(-c4cccc5oc6ccccc6c45)n3)c3c(c2)oc2ccccc23)cc1 ; c1ccc(-c2cc(-c3nc(-c4ccccc4)nc(-c4ccc5oc6ccccc6c5c4)n3)c3c(c2)oc2ccccc23)cc1 ; c1ccc(-c2cc(-c3nc(-c4ccccc4)nc(-c4ccc5c(c4)oc4ccccc45)n3)c3c(c2)oc2ccccc23)cc1 ; c1ccc(-c2cc(-c3nc(-c4ccccc4)nc(-c4cccc5c4oc4ccccc45)n3)c3c(c2)oc2ccccc23)cc1]

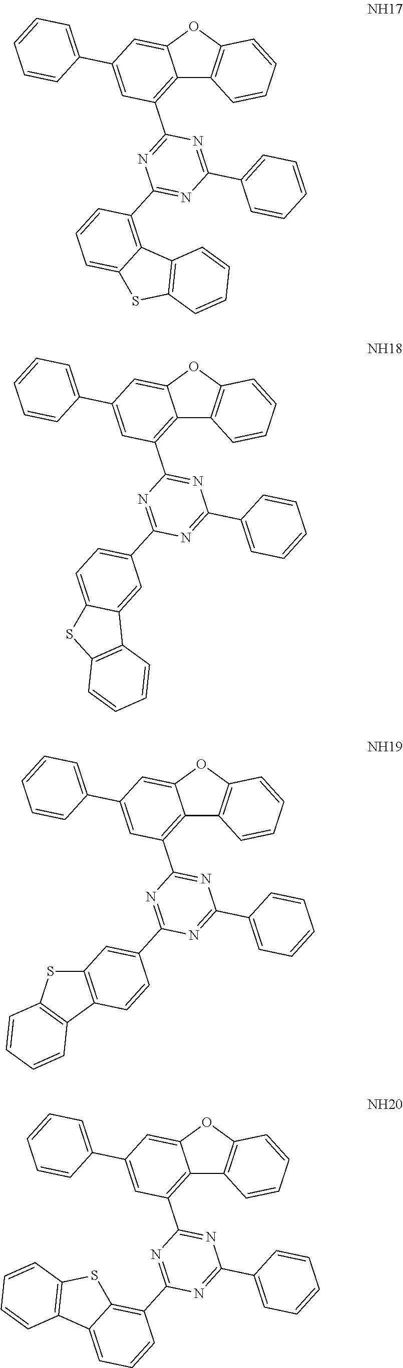 [SMILES: c1ccc(-c2cc(-c3nc(-c4ccccc4)nc(-c4cccc5sc6ccccc6c45)n3)c3c(c2)oc2ccccc23)cc1 ; c1ccc(-c2cc(-c3nc(-c4ccccc4)nc(-c4ccc5sc6ccccc6c5c4)n3)c3c(c2)oc2ccccc23)cc1 ; c1ccc(-c2cc(-c3nc(-c4ccccc4)nc(-c4ccc5c(c4)sc4ccccc45)n3)c3c(c2)oc2ccccc23)cc1 ; c1ccc(-c2cc(-c3nc(-c4ccccc4)nc(-c4cccc5c4sc4ccccc45)n3)c3c(c2)oc2ccccc23)cc1]

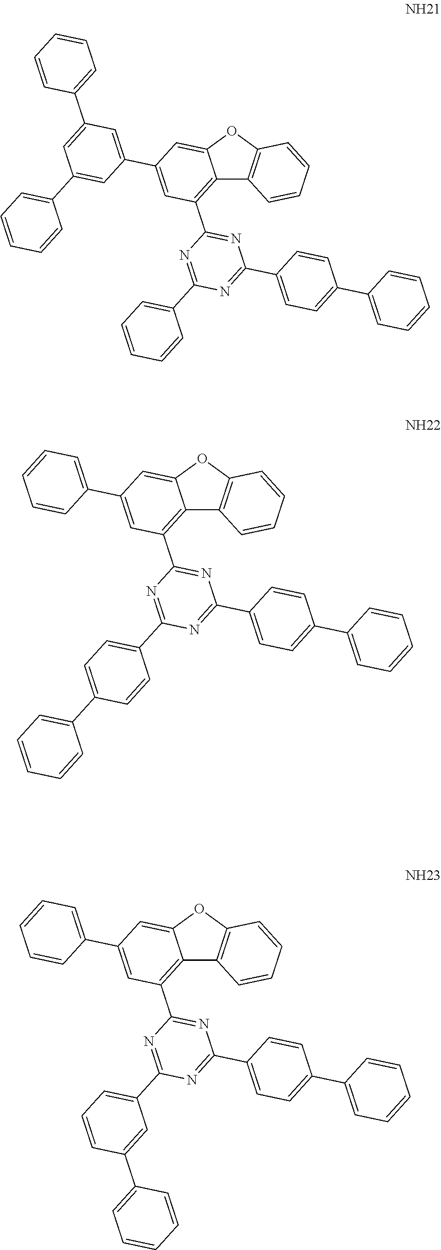 [SMILES: c1ccc(-c2ccc(-c3nc(-c4ccccc4)nc(-c4cc(-c5cc(-c6ccccc6)cc(-c6ccccc6)c5)cc5oc6ccccc6c45)n3)cc2)cc1 ; c1ccc(-c2ccc(-c3nc(-c4ccc(-c5ccccc5)cc4)nc(-c4cc(-c5ccccc5)cc5oc6ccccc6c45)n3)cc2)cc1 ; c1ccc(-c2ccc(-c3nc(-c4cccc(-c5ccccc5)c4)nc(-c4cc(-c5ccccc5)cc5oc6ccccc6c45)n3)cc2)cc1]

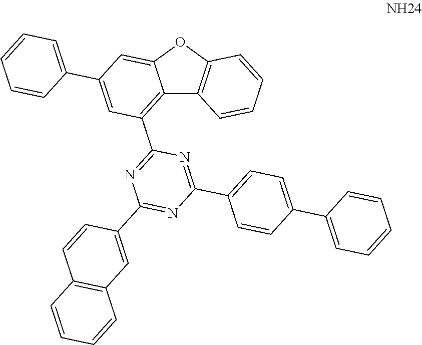 [SMILES: c1ccc(-c2ccc(-c3nc(-c4ccc5ccccc5c4)nc(-c4cc(-c5ccccc5)cc5oc6ccccc6c45)n3)cc2)cc1]

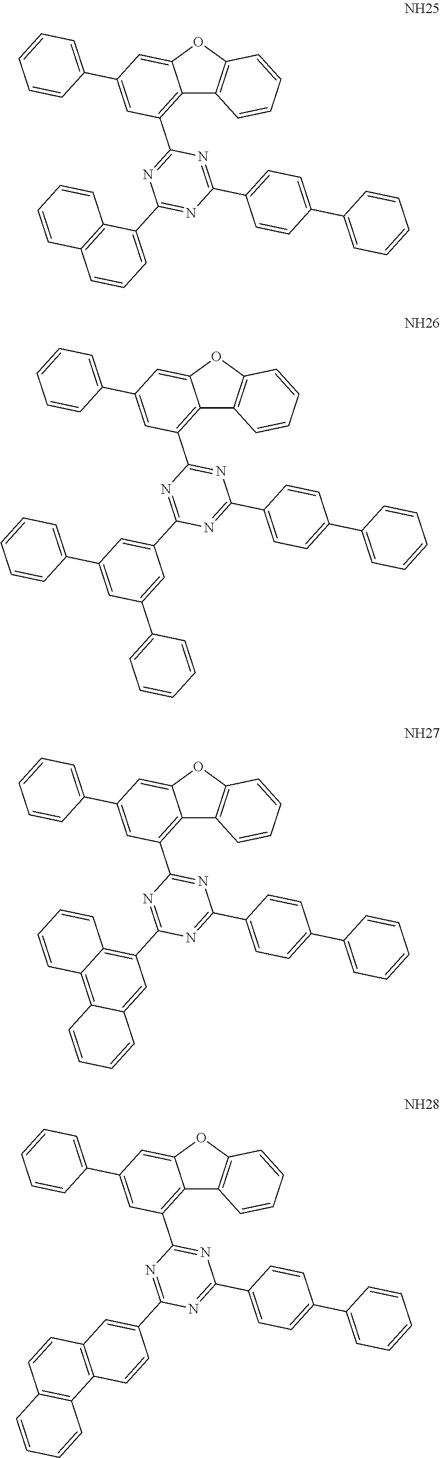 [SMILES: c1ccc(-c2ccc(-c3nc(-c4cccc5ccccc45)nc(-c4cc(-c5ccccc5)cc5oc6ccccc6c45)n3)cc2)cc1 ; c1ccc(-c2ccc(-c3nc(-c4cc(-c5ccccc5)cc(-c5ccccc5)c4)nc(-c4cc(-c5ccccc5)cc5oc6ccccc6c45)n3)cc2)cc1 ; c1ccc(-c2ccc(-c3nc(-c4cc5ccccc5c5ccccc45)nc(-c4cc(-c5ccccc5)cc5oc6ccccc6c45)n3)cc2)cc1 ; c1ccc(-c2ccc(-c3nc(-c4ccc5c(ccc6ccccc65)c4)nc(-c4cc(-c5ccccc5)cc5oc6ccccc6c45)n3)cc2)cc1]

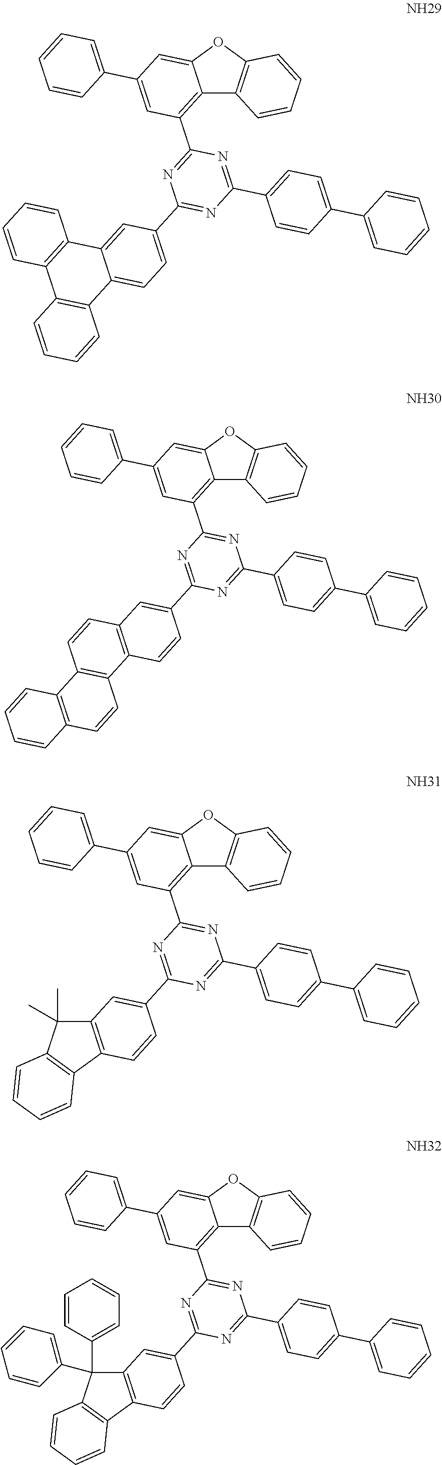 [SMILES: c1ccc(-c2ccc(-c3nc(-c4ccc5c6ccccc6c6ccccc6c5c4)nc(-c4cc(-c5ccccc5)cc5oc6ccccc6c45)n3)cc2)cc1 ; c1ccc(-c2ccc(-c3nc(-c4ccc5c(ccc6c7ccccc7ccc56)c4)nc(-c4cc(-c5ccccc5)cc5oc6ccccc6c45)n3)cc2)cc1 ; CC1(C)c2ccccc2-c2ccc(-c3nc(-c4ccc(-c5ccccc5)cc4)nc(-c4cc(-c5ccccc5)cc5oc6ccccc6c45)n3)cc21 ; c1ccc(-c2ccc(-c3nc(-c4ccc5c(c4)C(c4ccccc4)(c4ccccc4)c4ccccc4-5)nc(-c4cc(-c5ccccc5)cc5oc6ccccc6c45)n3)cc2)cc1]

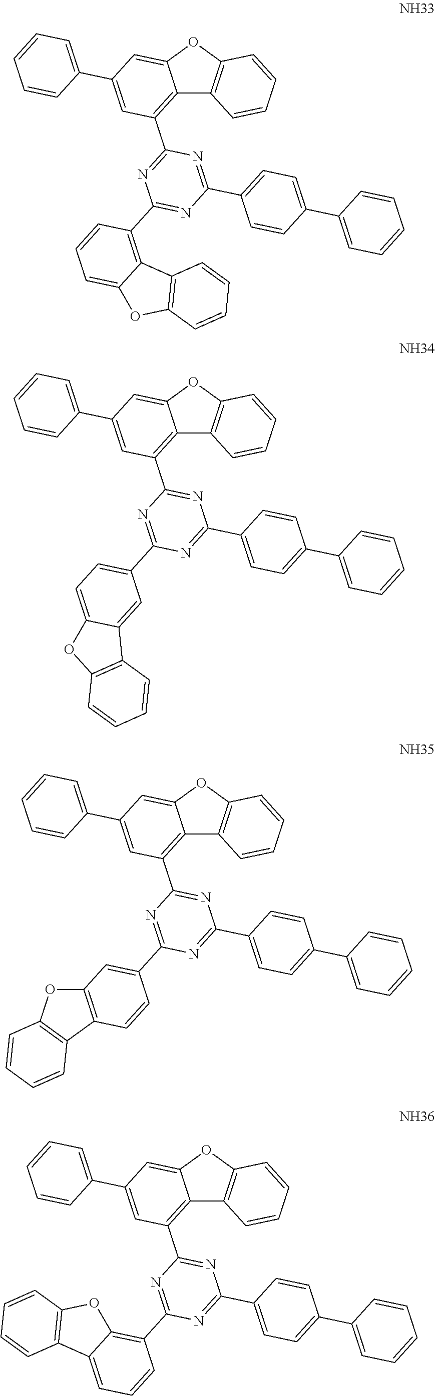 [SMILES: c1ccc(-c2ccc(-c3nc(-c4cccc5oc6ccccc6c45)nc(-c4cc(-c5ccccc5)cc5oc6ccccc6c45)n3)cc2)cc1 ; c1ccc(-c2ccc(-c3nc(-c4ccc5oc6ccccc6c5c4)nc(-c4cc(-c5ccccc5)cc5oc6ccccc6c45)n3)cc2)cc1 ; c1ccc(-c2ccc(-c3nc(-c4ccc5c(c4)oc4ccccc45)nc(-c4cc(-c5ccccc5)cc5oc6ccccc6c45)n3)cc2)cc1 ; c1ccc(-c2ccc(-c3nc(-c4cccc5c4oc4ccccc45)nc(-c4cc(-c5ccccc5)cc5oc6ccccc6c45)n3)cc2)cc1]

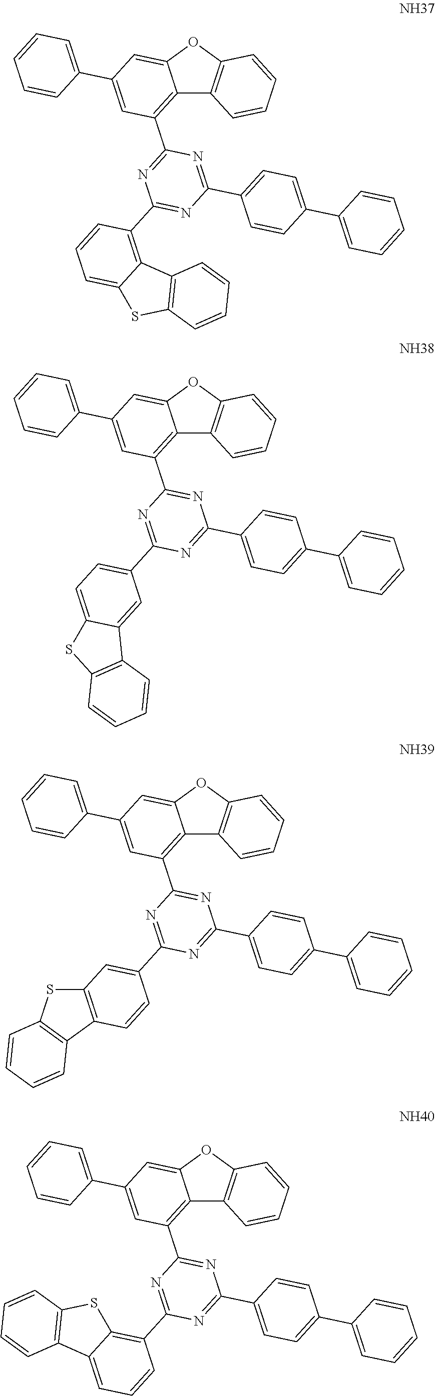 [SMILES: c1ccc(-c2ccc(-c3nc(-c4cc(-c5ccccc5)cc5oc6ccccc6c45)nc(-c4cccc5sc6ccccc6c45)n3)cc2)cc1 ; c1ccc(-c2ccc(-c3nc(-c4ccc5sc6ccccc6c5c4)nc(-c4cc(-c5ccccc5)cc5oc6ccccc6c45)n3)cc2)cc1 ; c1ccc(-c2ccc(-c3nc(-c4ccc5c(c4)sc4ccccc45)nc(-c4cc(-c5ccccc5)cc5oc6ccccc6c45)n3)cc2)cc1 ; c1ccc(-c2ccc(-c3nc(-c4cccc5c4sc4ccccc45)nc(-c4cc(-c5ccccc5)cc5oc6ccccc6c45)n3)cc2)cc1]

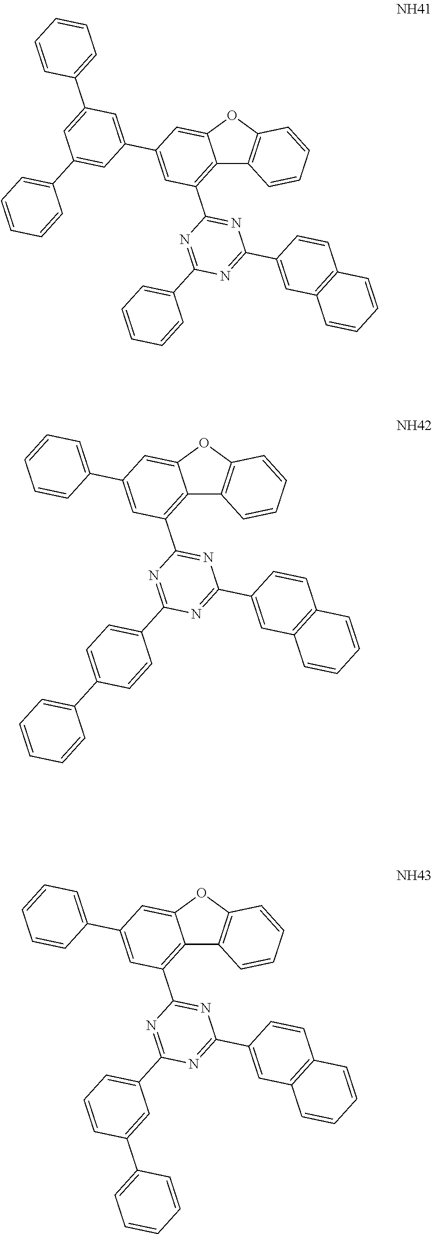 [SMILES: c1ccc(-c2cc(-c3ccccc3)cc(-c3cc(-c4nc(-c5ccccc5)nc(-c5ccc6ccccc6c5)n4)c4c(c3)oc3ccccc34)c2)cc1 ; c1ccc(-c2ccc(-c3nc(-c4ccc5ccccc5c4)nc(-c4cc(-c5ccccc5)cc5oc6ccccc6c45)n3)cc2)cc1 ; c1ccc(-c2cccc(-c3nc(-c4ccc5ccccc5c4)nc(-c4cc(-c5ccccc5)cc5oc6ccccc6c45)n3)c2)cc1]

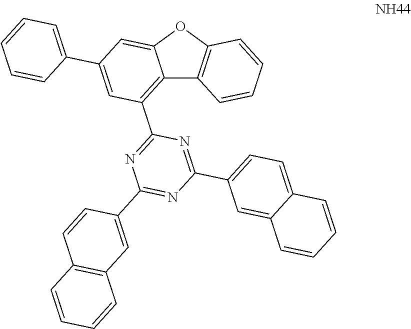 [SMILES: c1ccc(-c2cc(-c3nc(-c4ccc5ccccc5c4)nc(-c4ccc5ccccc5c4)n3)c3c(c2)oc2ccccc23)cc1]

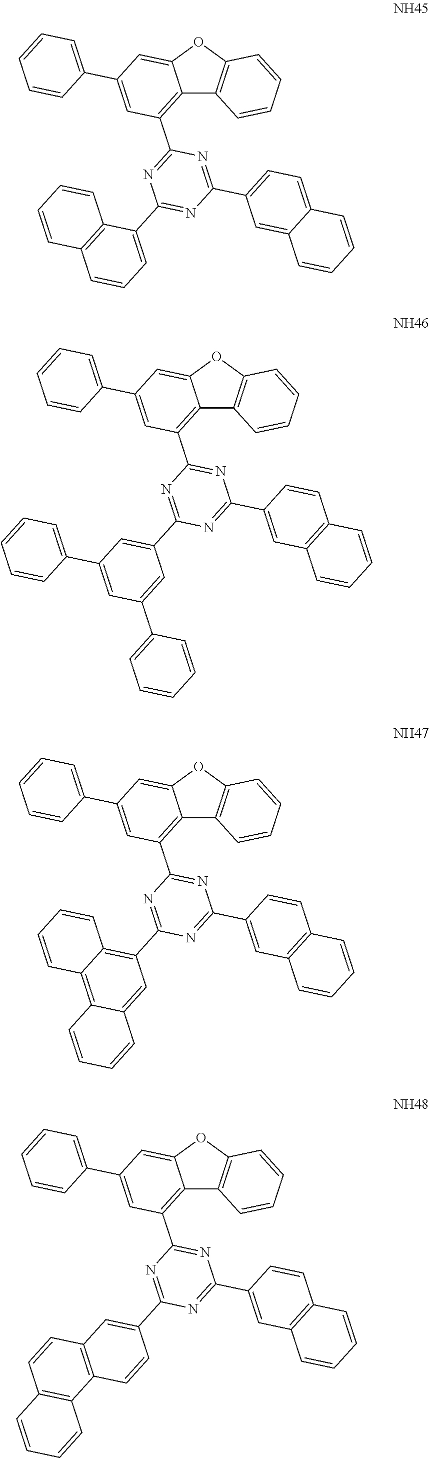 [SMILES: c1ccc(-c2cc(-c3nc(-c4ccc5ccccc5c4)nc(-c4cccc5ccccc45)n3)c3c(c2)oc2ccccc23)cc1 ; c1ccc(-c2cc(-c3ccccc3)cc(-c3nc(-c4ccc5ccccc5c4)nc(-c4cc(-c5ccccc5)cc5oc6ccccc6c45)n3)c2)cc1 ; c1ccc(-c2cc(-c3nc(-c4ccc5ccccc5c4)nc(-c4cc5ccccc5c5ccccc45)n3)c3c(c2)oc2ccccc23)cc1 ; c1ccc(-c2cc(-c3nc(-c4ccc5ccccc5c4)nc(-c4ccc5c(ccc6ccccc65)c4)n3)c3c(c2)oc2ccccc23)cc1]

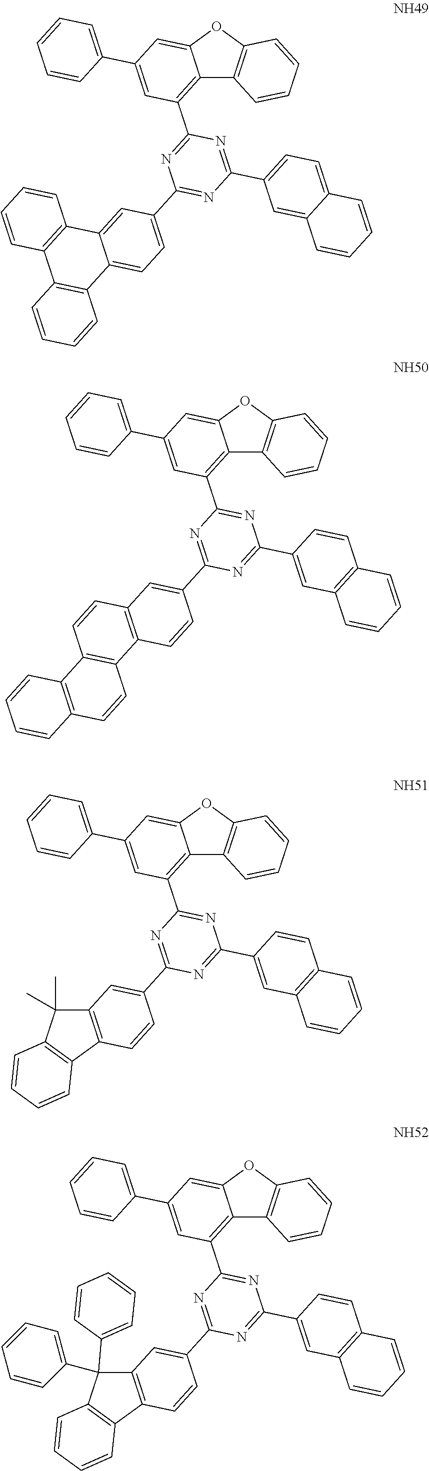 [SMILES: c1ccc(-c2cc(-c3nc(-c4ccc5ccccc5c4)nc(-c4ccc5c6ccccc6c6ccccc6c5c4)n3)c3c(c2)oc2ccccc23)cc1 ; c1ccc(-c2cc(-c3nc(-c4ccc5ccccc5c4)nc(-c4ccc5c(ccc6c7ccccc7ccc56)c4)n3)c3c(c2)oc2ccccc23)cc1 ; CC1(C)c2ccccc2-c2ccc(-c3nc(-c4ccc5ccccc5c4)nc(-c4cc(-c5ccccc5)cc5oc6ccccc6c45)n3)cc21 ; c1ccc(-c2cc(-c3nc(-c4ccc5c(c4)C(c4ccccc4)(c4ccccc4)c4ccccc4-5)nc(-c4ccc5ccccc5c4)n3)c3c(c2)oc2ccccc23)cc1]

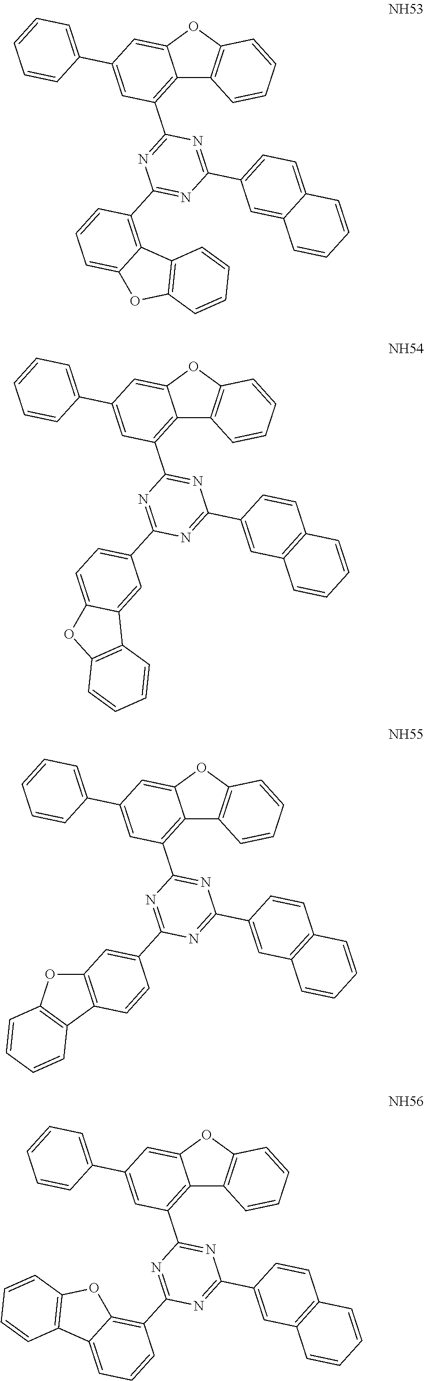 [SMILES: c1ccc(-c2cc(-c3nc(-c4ccc5ccccc5c4)nc(-c4cccc5oc6ccccc6c45)n3)c3c(c2)oc2ccccc23)cc1 ; c1ccc(-c2cc(-c3nc(-c4ccc5ccccc5c4)nc(-c4ccc5oc6ccccc6c5c4)n3)c3c(c2)oc2ccccc23)cc1 ; c1ccc(-c2cc(-c3nc(-c4ccc5ccccc5c4)nc(-c4ccc5c(c4)oc4ccccc45)n3)c3c(c2)oc2ccccc23)cc1 ; c1ccc(-c2cc(-c3nc(-c4ccc5ccccc5c4)nc(-c4cccc5c4oc4ccccc45)n3)c3c(c2)oc2ccccc23)cc1]

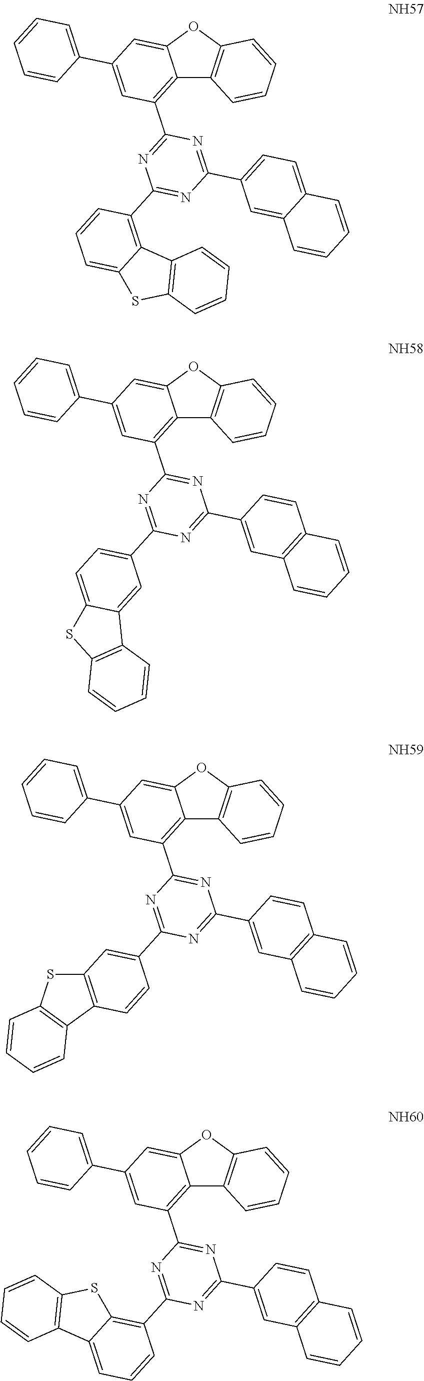 [SMILES: c1ccc(-c2cc(-c3nc(-c4ccc5ccccc5c4)nc(-c4cccc5sc6ccccc6c45)n3)c3c(c2)oc2ccccc23)cc1 ; c1ccc(-c2cc(-c3nc(-c4ccc5ccccc5c4)nc(-c4ccc5sc6ccccc6c5c4)n3)c3c(c2)oc2ccccc23)cc1 ; c1ccc(-c2cc(-c3nc(-c4ccc5ccccc5c4)nc(-c4ccc5c(c4)sc4ccccc45)n3)c3c(c2)oc2ccccc23)cc1 ; c1ccc(-c2cc(-c3nc(-c4ccc5ccccc5c4)nc(-c4cccc5c4sc4ccccc45)n3)c3c(c2)oc2ccccc23)cc1]

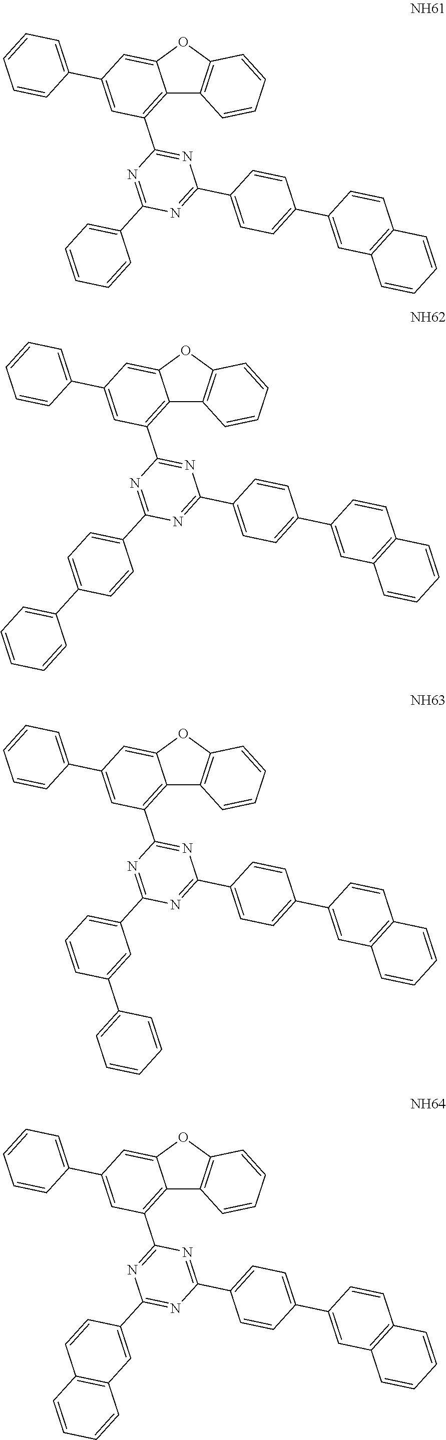 [SMILES: c1ccc(-c2cc(-c3nc(-c4ccccc4)nc(-c4ccc(-c5ccc6ccccc6c5)cc4)n3)c3c(c2)oc2ccccc23)cc1 ; c1ccc(-c2ccc(-c3nc(-c4ccc(-c5ccc6ccccc6c5)cc4)nc(-c4cc(-c5ccccc5)cc5oc6ccccc6c45)n3)cc2)cc1 ; c1ccc(-c2cccc(-c3nc(-c4ccc(-c5ccc6ccccc6c5)cc4)nc(-c4cc(-c5ccccc5)cc5oc6ccccc6c45)n3)c2)cc1 ; c1ccc(-c2cc(-c3nc(-c4ccc(-c5ccc6ccccc6c5)cc4)nc(-c4ccc5ccccc5c4)n3)c3c(c2)oc2ccccc23)cc1]

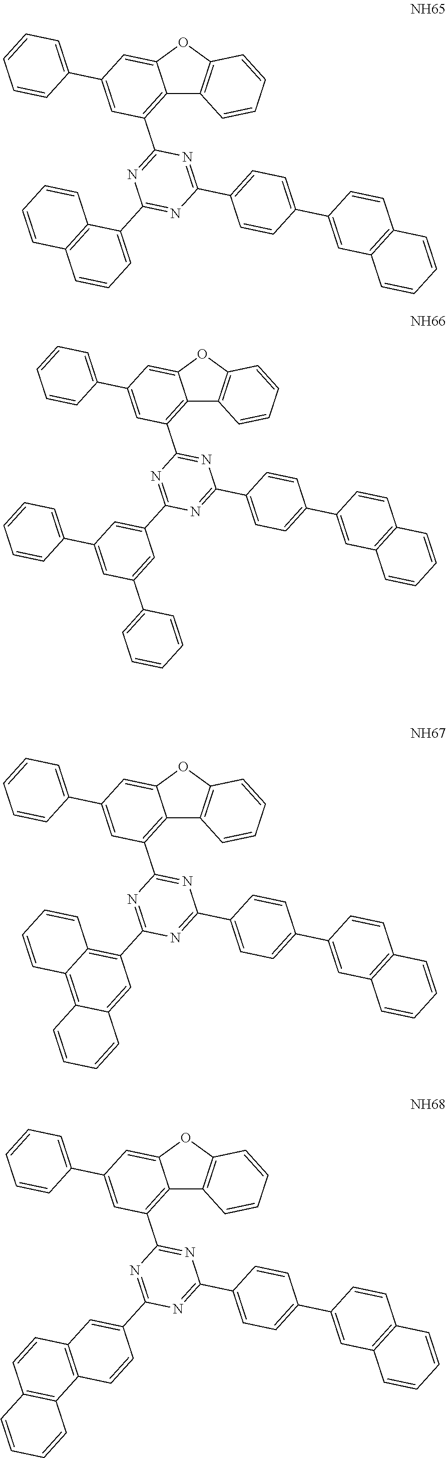 [SMILES: c1ccc(-c2cc(-c3nc(-c4ccc(-c5ccc6ccccc6c5)cc4)nc(-c4cccc5ccccc45)n3)c3c(c2)oc2ccccc23)cc1 ; c1ccc(-c2cc(-c3ccccc3)cc(-c3nc(-c4ccc(-c5ccc6ccccc6c5)cc4)nc(-c4cc(-c5ccccc5)cc5oc6ccccc6c45)n3)c2)cc1 ; c1ccc(-c2cc(-c3nc(-c4ccc(-c5ccc6ccccc6c5)cc4)nc(-c4cc5ccccc5c5ccccc45)n3)c3c(c2)oc2ccccc23)cc1 ; c1ccc(-c2cc(-c3nc(-c4ccc(-c5ccc6ccccc6c5)cc4)nc(-c4ccc5c(ccc6ccccc65)c4)n3)c3c(c2)oc2ccccc23)cc1]

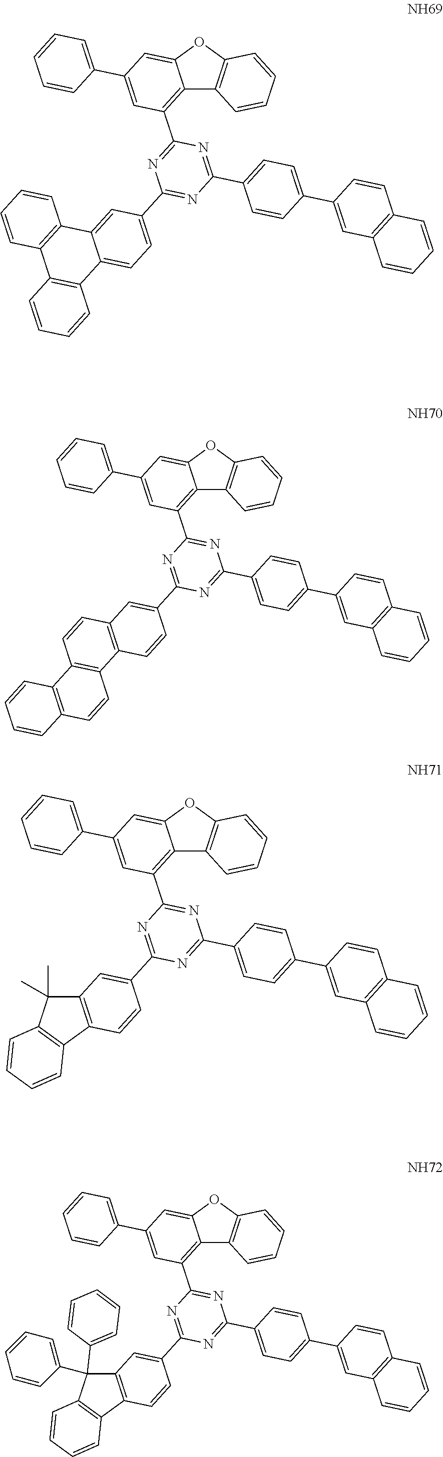 [SMILES: c1ccc(-c2cc(-c3nc(-c4ccc(-c5ccc6ccccc6c5)cc4)nc(-c4ccc5c6ccccc6c6ccccc6c5c4)n3)c3c(c2)oc2ccccc23)cc1 ; c1ccc(-c2cc(-c3nc(-c4ccc(-c5ccc6ccccc6c5)cc4)nc(-c4ccc5c(ccc6c7ccccc7ccc56)c4)n3)c3c(c2)oc2ccccc23)cc1 ; CC1(C)c2ccccc2-c2ccc(-c3nc(-c4ccc(-c5ccc6ccccc6c5)cc4)nc(-c4cc(-c5ccccc5)cc5oc6ccccc6c45)n3)cc21 ; c1ccc(-c2cc(-c3nc(-c4ccc(-c5ccc6ccccc6c5)cc4)nc(-c4ccc5c(c4)C(c4ccccc4)(c4ccccc4)c4ccccc4-5)n3)c3c(c2)oc2ccccc23)cc1]

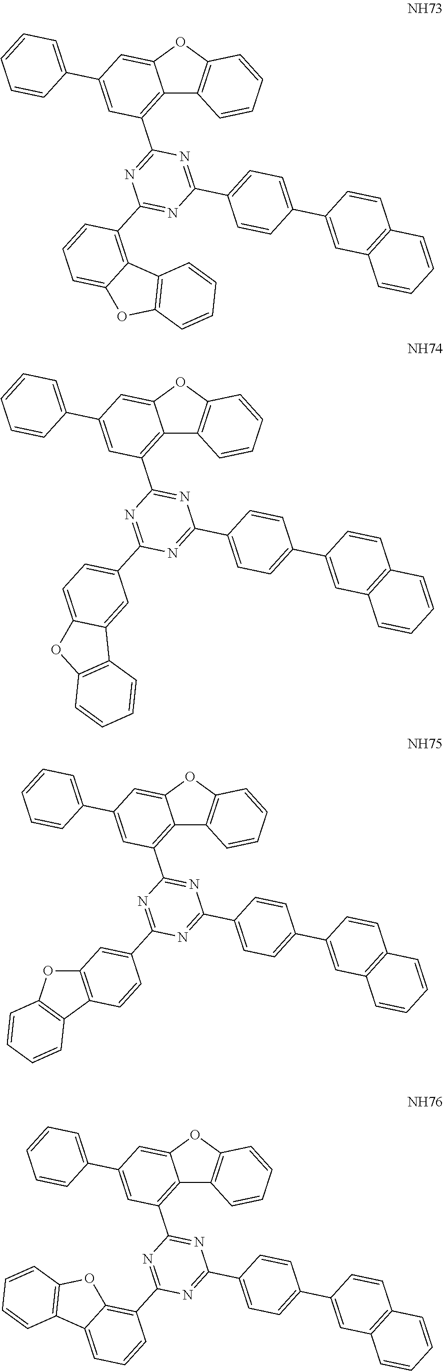 [SMILES: c1ccc(-c2cc(-c3nc(-c4ccc(-c5ccc6ccccc6c5)cc4)nc(-c4cccc5oc6ccccc6c45)n3)c3c(c2)oc2ccccc23)cc1 ; c1ccc(-c2cc(-c3nc(-c4ccc(-c5ccc6ccccc6c5)cc4)nc(-c4ccc5oc6ccccc6c5c4)n3)c3c(c2)oc2ccccc23)cc1 ; c1ccc(-c2cc(-c3nc(-c4ccc(-c5ccc6ccccc6c5)cc4)nc(-c4ccc5c(c4)oc4ccccc45)n3)c3c(c2)oc2ccccc23)cc1 ; c1ccc(-c2cc(-c3nc(-c4ccc(-c5ccc6ccccc6c5)cc4)nc(-c4cccc5c4oc4ccccc45)n3)c3c(c2)oc2ccccc23)cc1]

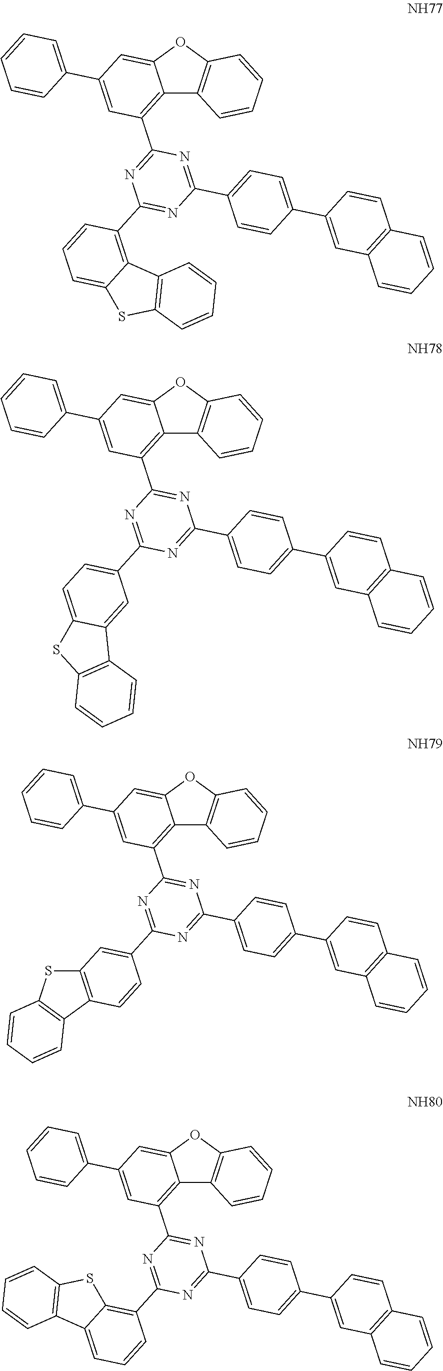 [SMILES: c1ccc(-c2cc(-c3nc(-c4ccc(-c5ccc6ccccc6c5)cc4)nc(-c4cccc5sc6ccccc6c45)n3)c3c(c2)oc2ccccc23)cc1 ; c1ccc(-c2cc(-c3nc(-c4ccc(-c5ccc6ccccc6c5)cc4)nc(-c4ccc5sc6ccccc6c5c4)n3)c3c(c2)oc2ccccc23)cc1 ; c1ccc(-c2cc(-c3nc(-c4ccc(-c5ccc6ccccc6c5)cc4)nc(-c4ccc5c(c4)sc4ccccc45)n3)c3c(c2)oc2ccccc23)cc1 ; c1ccc(-c2cc(-c3nc(-c4ccc(-c5ccc6ccccc6c5)cc4)nc(-c4cccc5c4sc4ccccc45)n3)c3c(c2)oc2ccccc23)cc1]

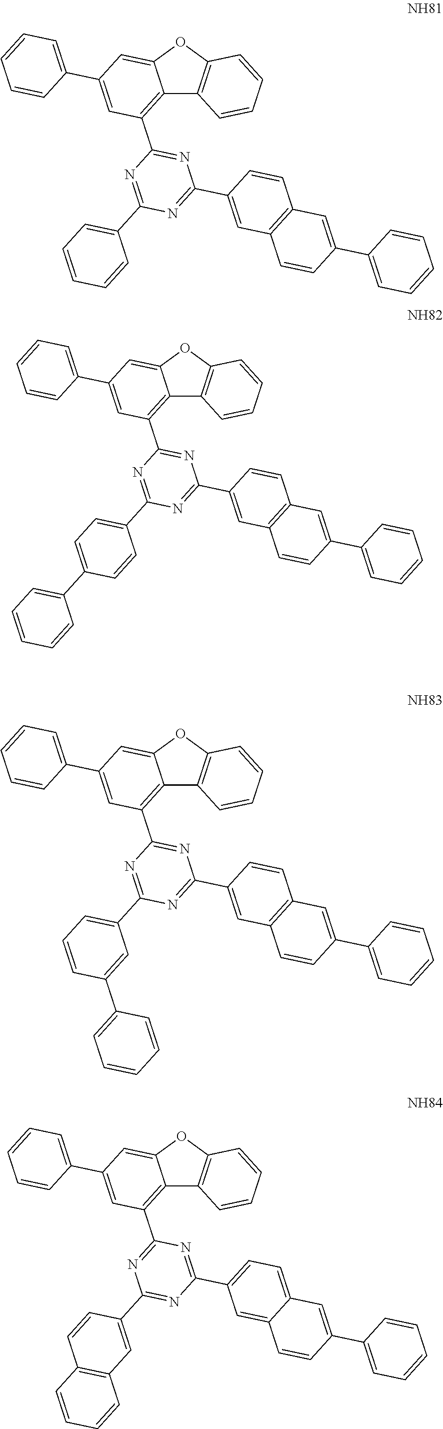 [SMILES: c1ccc(-c2ccc3cc(-c4nc(-c5ccccc5)nc(-c5cc(-c6ccccc6)cc6oc7ccccc7c56)n4)ccc3c2)cc1 ; c1ccc(-c2ccc(-c3nc(-c4ccc5cc(-c6ccccc6)ccc5c4)nc(-c4cc(-c5ccccc5)cc5oc6ccccc6c45)n3)cc2)cc1 ; c1ccc(-c2cccc(-c3nc(-c4ccc5cc(-c6ccccc6)ccc5c4)nc(-c4cc(-c5ccccc5)cc5oc6ccccc6c45)n3)c2)cc1 ; c1ccc(-c2ccc3cc(-c4nc(-c5ccc6ccccc6c5)nc(-c5cc(-c6ccccc6)cc6oc7ccccc7c56)n4)ccc3c2)cc1]

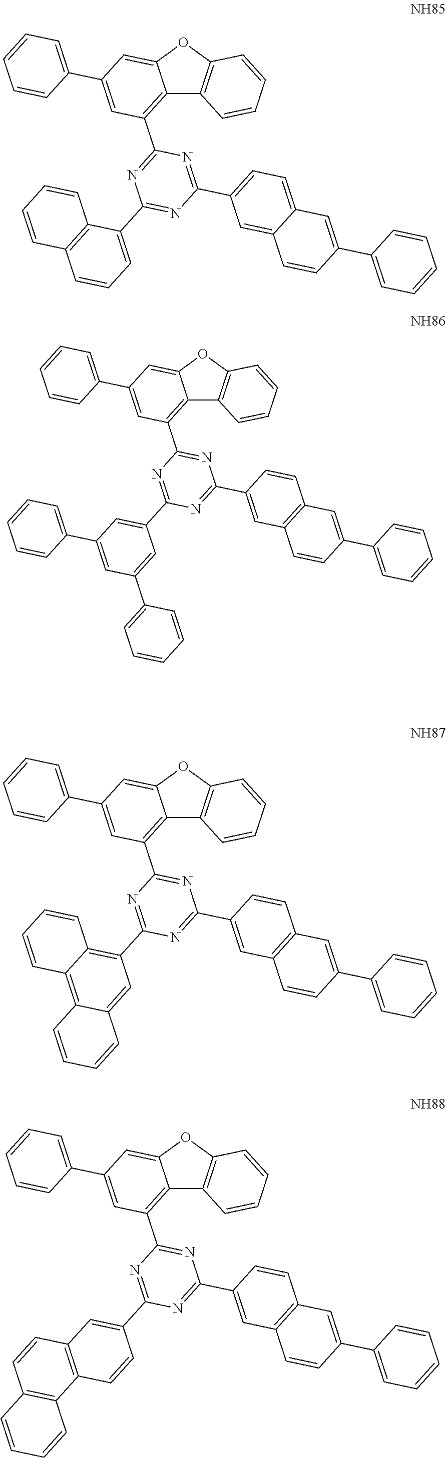 [SMILES: c1ccc(-c2ccc3cc(-c4nc(-c5cccc6ccccc56)nc(-c5cc(-c6ccccc6)cc6oc7ccccc7c56)n4)ccc3c2)cc1 ; c1ccc(-c2cc(-c3ccccc3)cc(-c3nc(-c4ccc5cc(-c6ccccc6)ccc5c4)nc(-c4cc(-c5ccccc5)cc5oc6ccccc6c45)n3)c2)cc1 ; c1ccc(-c2ccc3cc(-c4nc(-c5cc6ccccc6c6ccccc56)nc(-c5cc(-c6ccccc6)cc6oc7ccccc7c56)n4)ccc3c2)cc1 ; c1ccc(-c2ccc3cc(-c4nc(-c5ccc6c(ccc7ccccc76)c5)nc(-c5cc(-c6ccccc6)cc6oc7ccccc7c56)n4)ccc3c2)cc1]

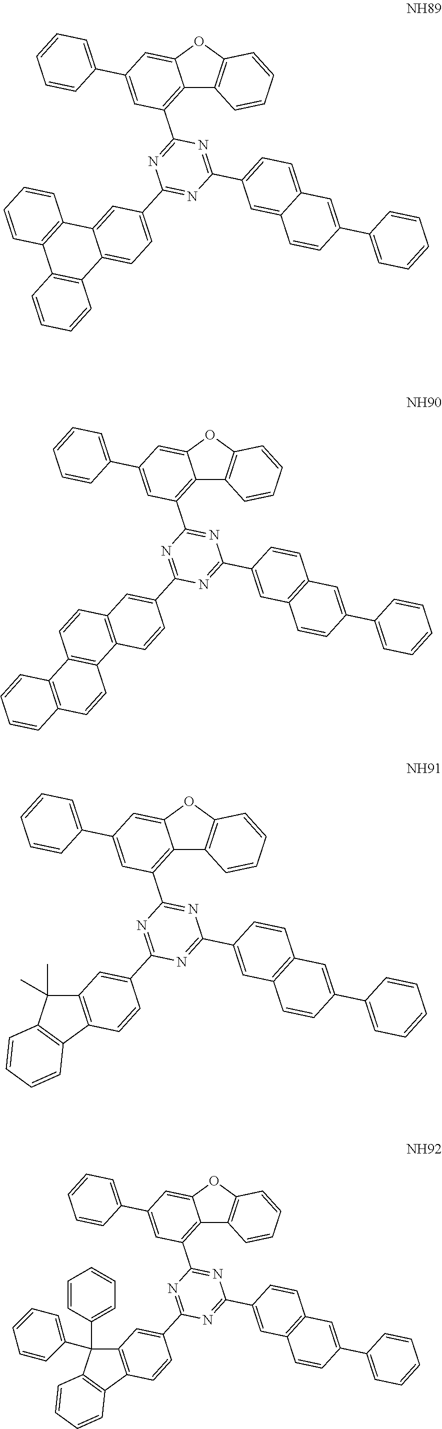 [SMILES: c1ccc(-c2ccc3cc(-c4nc(-c5ccc6c7ccccc7c7ccccc7c6c5)nc(-c5cc(-c6ccccc6)cc6oc7ccccc7c56)n4)ccc3c2)cc1 ; c1ccc(-c2ccc3cc(-c4nc(-c5ccc6c(ccc7c8ccccc8ccc67)c5)nc(-c5cc(-c6ccccc6)cc6oc7ccccc7c56)n4)ccc3c2)cc1 ; CC1(C)c2ccccc2-c2ccc(-c3nc(-c4ccc5cc(-c6ccccc6)ccc5c4)nc(-c4cc(-c5ccccc5)cc5oc6ccccc6c45)n3)cc21 ; c1ccc(-c2ccc3cc(-c4nc(-c5ccc6c(c5)C(c5ccccc5)(c5ccccc5)c5ccccc5-6)nc(-c5cc(-c6ccccc6)cc6oc7ccccc7c56)n4)ccc3c2)cc1]

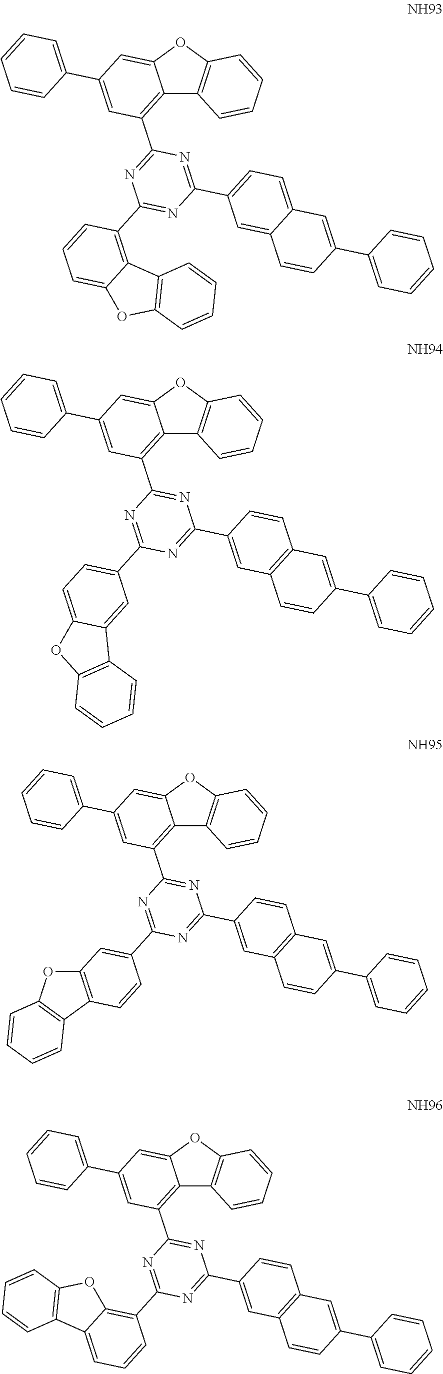 [SMILES: c1ccc(-c2ccc3cc(-c4nc(-c5cccc6oc7ccccc7c56)nc(-c5cc(-c6ccccc6)cc6oc7ccccc7c56)n4)ccc3c2)cc1 ; c1ccc(-c2ccc3cc(-c4nc(-c5ccc6oc7ccccc7c6c5)nc(-c5cc(-c6ccccc6)cc6oc7ccccc7c56)n4)ccc3c2)cc1 ; c1ccc(-c2ccc3cc(-c4nc(-c5ccc6c(c5)oc5ccccc56)nc(-c5cc(-c6ccccc6)cc6oc7ccccc7c56)n4)ccc3c2)cc1 ; c1ccc(-c2ccc3cc(-c4nc(-c5cccc6c5oc5ccccc56)nc(-c5cc(-c6ccccc6)cc6oc7ccccc7c56)n4)ccc3c2)cc1]

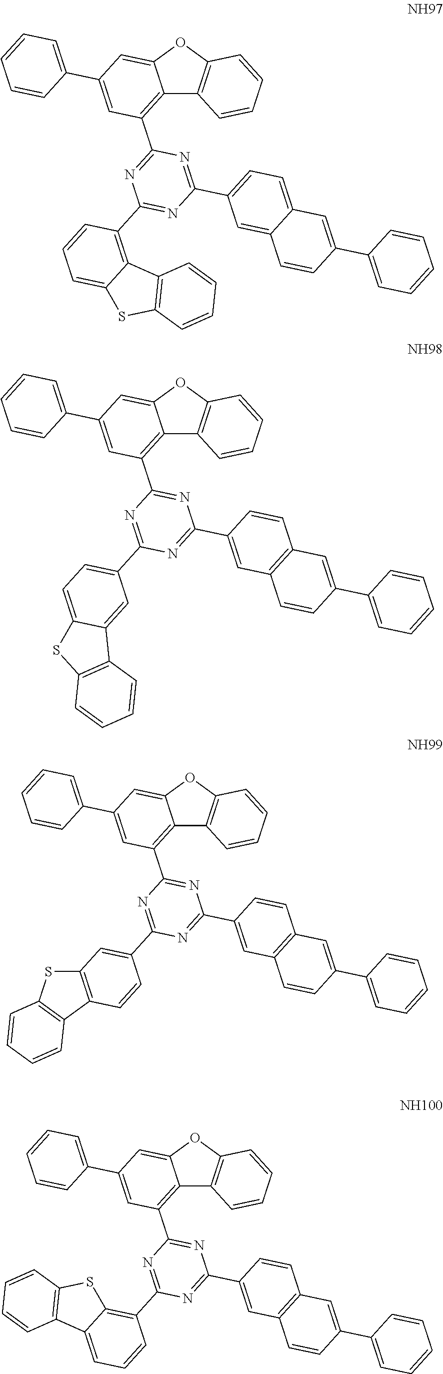 [SMILES: c1ccc(-c2ccc3cc(-c4nc(-c5cc(-c6ccccc6)cc6oc7ccccc7c56)nc(-c5cccc6sc7ccccc7c56)n4)ccc3c2)cc1 ; c1ccc(-c2ccc3cc(-c4nc(-c5ccc6sc7ccccc7c6c5)nc(-c5cc(-c6ccccc6)cc6oc7ccccc7c56)n4)ccc3c2)cc1 ; c1ccc(-c2ccc3cc(-c4nc(-c5ccc6c(c5)sc5ccccc56)nc(-c5cc(-c6ccccc6)cc6oc7ccccc7c56)n4)ccc3c2)cc1 ; c1ccc(-c2ccc3cc(-c4nc(-c5cccc6c5sc5ccccc56)nc(-c5cc(-c6ccccc6)cc6oc7ccccc7c56)n4)ccc3c2)cc1]

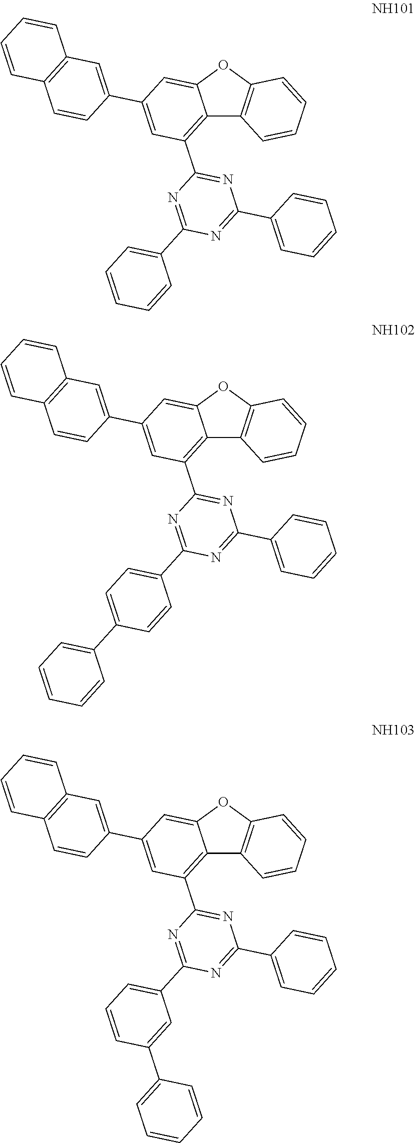 [SMILES: c1ccc(-c2nc(-c3ccccc3)nc(-c3cc(-c4ccc5ccccc5c4)cc4oc5ccccc5c34)n2)cc1 ; c1ccc(-c2ccc(-c3nc(-c4ccccc4)nc(-c4cc(-c5ccc6ccccc6c5)cc5oc6ccccc6c45)n3)cc2)cc1 ; c1ccc(-c2cccc(-c3nc(-c4ccccc4)nc(-c4cc(-c5ccc6ccccc6c5)cc5oc6ccccc6c45)n3)c2)cc1]

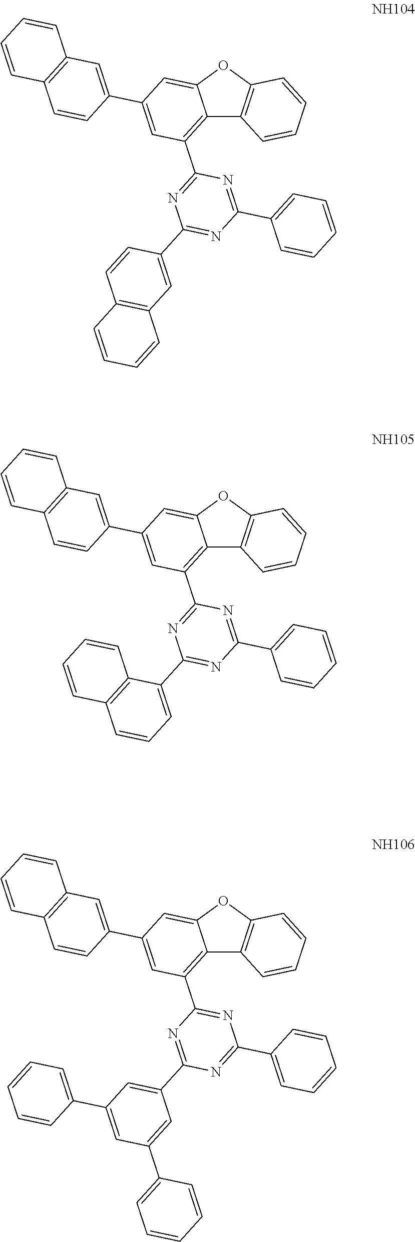 [SMILES: c1ccc(-c2nc(-c3ccc4ccccc4c3)nc(-c3cc(-c4ccc5ccccc5c4)cc4oc5ccccc5c34)n2)cc1 ; c1ccc(-c2nc(-c3cccc4ccccc34)nc(-c3cc(-c4ccc5ccccc5c4)cc4oc5ccccc5c34)n2)cc1 ; c1ccc(-c2cc(-c3ccccc3)cc(-c3nc(-c4ccccc4)nc(-c4cc(-c5ccc6ccccc6c5)cc5oc6ccccc6c45)n3)c2)cc1]

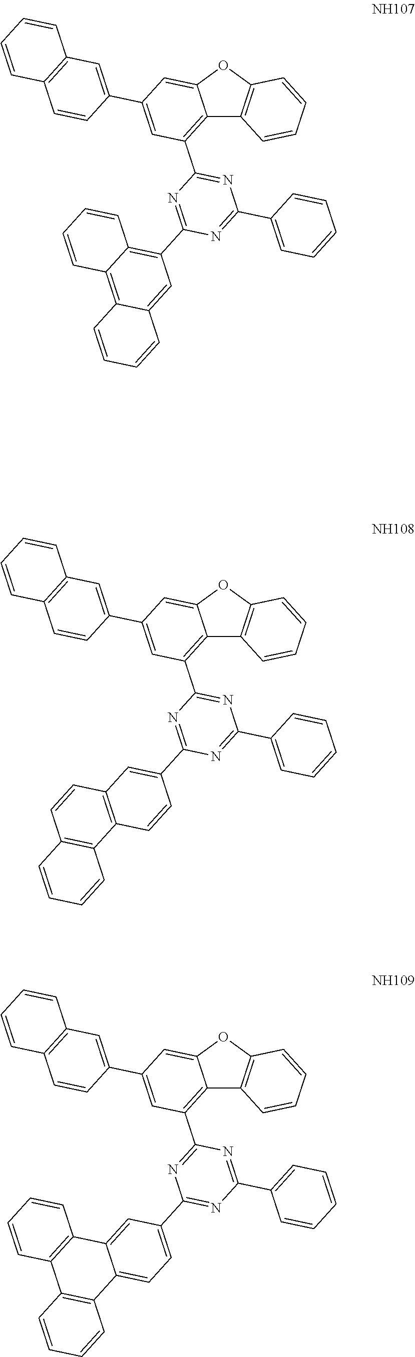 [SMILES: c1ccc(-c2nc(-c3cc4ccccc4c4ccccc34)nc(-c3cc(-c4ccc5ccccc5c4)cc4oc5ccccc5c34)n2)cc1 ; c1ccc(-c2nc(-c3ccc4c(ccc5ccccc54)c3)nc(-c3cc(-c4ccc5ccccc5c4)cc4oc5ccccc5c34)n2)cc1 ; c1ccc(-c2nc(-c3ccc4c5ccccc5c5ccccc5c4c3)nc(-c3cc(-c4ccc5ccccc5c4)cc4oc5ccccc5c34)n2)cc1]

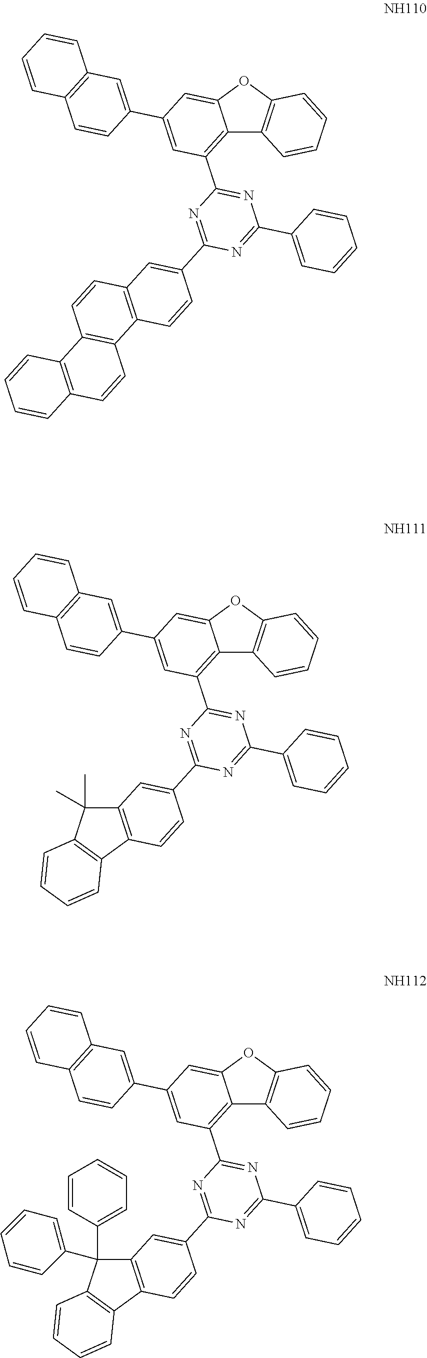 [SMILES: c1ccc(-c2nc(-c3ccc4c(ccc5c6ccccc6ccc45)c3)nc(-c3cc(-c4ccc5ccccc5c4)cc4oc5ccccc5c34)n2)cc1 ; CC1(C)c2ccccc2-c2ccc(-c3nc(-c4ccccc4)nc(-c4cc(-c5ccc6ccccc6c5)cc5oc6ccccc6c45)n3)cc21 ; c1ccc(-c2nc(-c3ccc4c(c3)C(c3ccccc3)(c3ccccc3)c3ccccc3-4)nc(-c3cc(-c4ccc5ccccc5c4)cc4oc5ccccc5c34)n2)cc1]

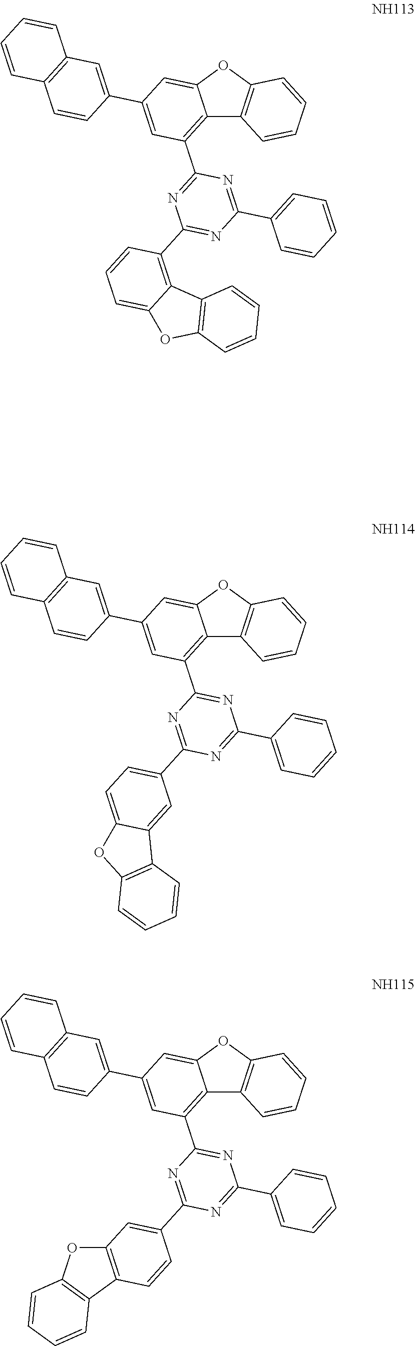 [SMILES: c1ccc(-c2nc(-c3cccc4oc5ccccc5c34)nc(-c3cc(-c4ccc5ccccc5c4)cc4oc5ccccc5c34)n2)cc1 ; c1ccc(-c2nc(-c3ccc4oc5ccccc5c4c3)nc(-c3cc(-c4ccc5ccccc5c4)cc4oc5ccccc5c34)n2)cc1 ; c1ccc(-c2nc(-c3ccc4c(c3)oc3ccccc34)nc(-c3cc(-c4ccc5ccccc5c4)cc4oc5ccccc5c34)n2)cc1]

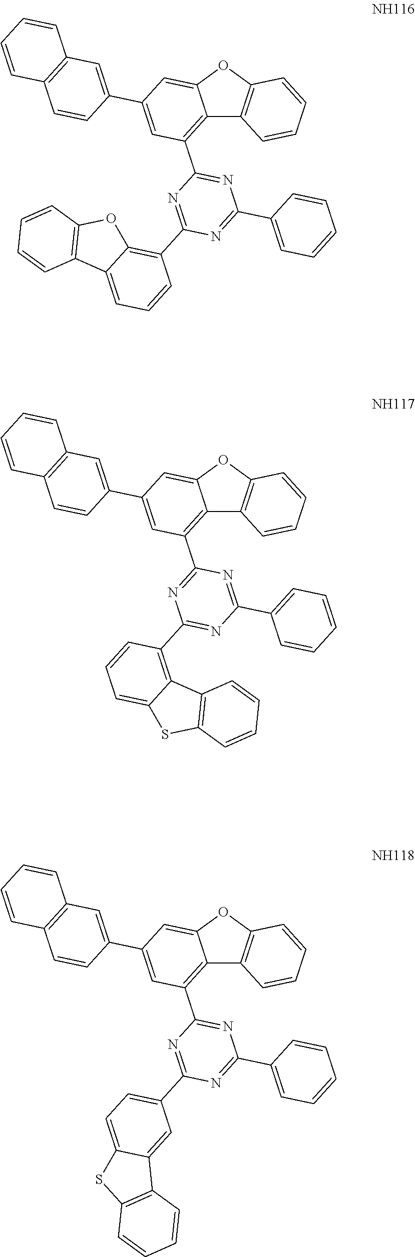 [SMILES: c1ccc(-c2nc(-c3cccc4c3oc3ccccc34)nc(-c3cc(-c4ccc5ccccc5c4)cc4oc5ccccc5c34)n2)cc1 ; c1ccc(-c2nc(-c3cc(-c4ccc5ccccc5c4)cc4oc5ccccc5c34)nc(-c3cccc4sc5ccccc5c34)n2)cc1 ; c1ccc(-c2nc(-c3ccc4sc5ccccc5c4c3)nc(-c3cc(-c4ccc5ccccc5c4)cc4oc5ccccc5c34)n2)cc1]

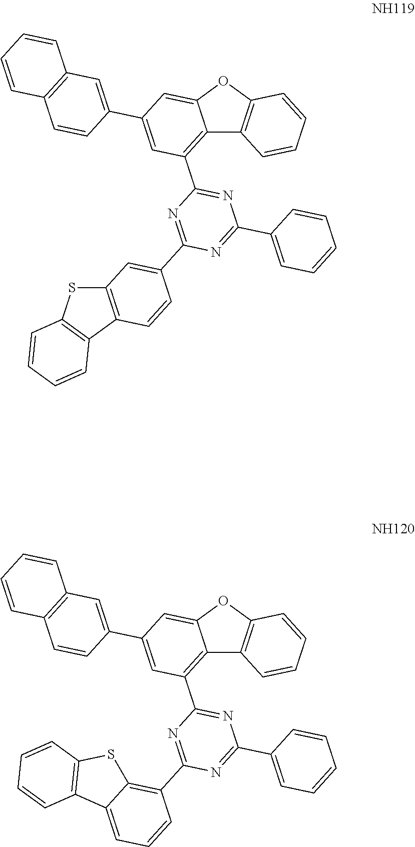 [SMILES: c1ccc(-c2nc(-c3ccc4c(c3)sc3ccccc34)nc(-c3cc(-c4ccc5ccccc5c4)cc4oc5ccccc5c34)n2)cc1 ; c1ccc(-c2nc(-c3cccc4c3sc3ccccc34)nc(-c3cc(-c4ccc5ccccc5c4)cc4oc5ccccc5c34)n2)cc1]

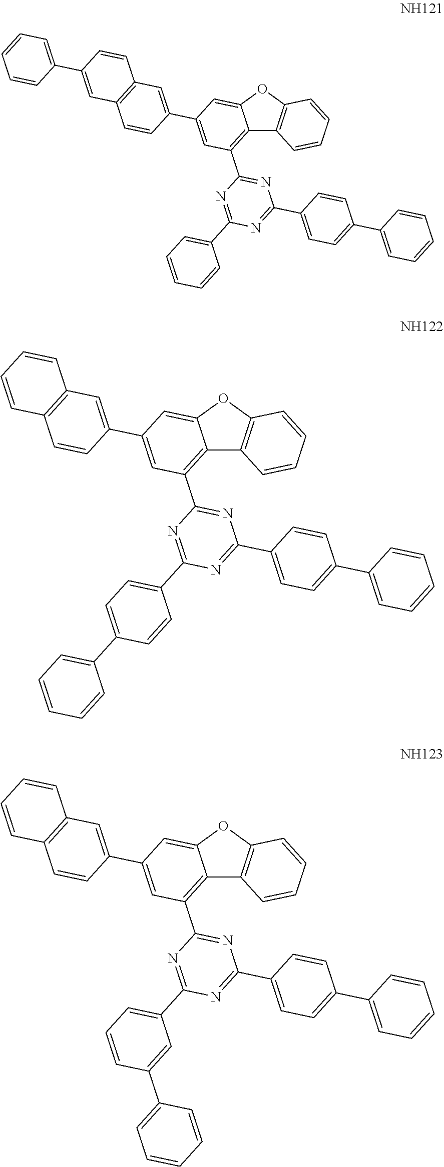 [SMILES: c1ccc(-c2ccc(-c3nc(-c4ccccc4)nc(-c4cc(-c5ccc6cc(-c7ccccc7)ccc6c5)cc5oc6ccccc6c45)n3)cc2)cc1 ; c1ccc(-c2ccc(-c3nc(-c4ccc(-c5ccccc5)cc4)nc(-c4cc(-c5ccc6ccccc6c5)cc5oc6ccccc6c45)n3)cc2)cc1 ; c1ccc(-c2ccc(-c3nc(-c4cccc(-c5ccccc5)c4)nc(-c4cc(-c5ccc6ccccc6c5)cc5oc6ccccc6c45)n3)cc2)cc1]

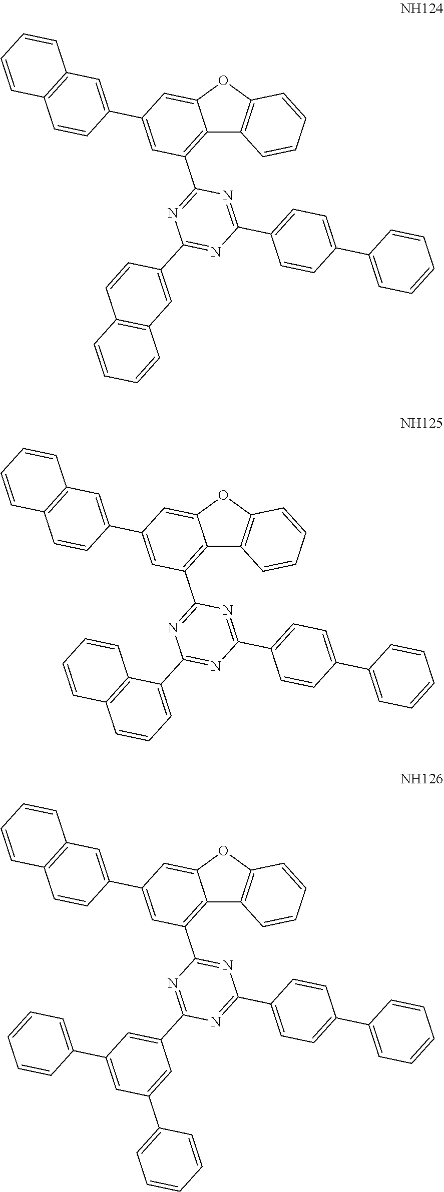 [SMILES: c1ccc(-c2ccc(-c3nc(-c4ccc5ccccc5c4)nc(-c4cc(-c5ccc6ccccc6c5)cc5oc6ccccc6c45)n3)cc2)cc1 ; c1ccc(-c2ccc(-c3nc(-c4cccc5ccccc45)nc(-c4cc(-c5ccc6ccccc6c5)cc5oc6ccccc6c45)n3)cc2)cc1 ; c1ccc(-c2ccc(-c3nc(-c4cc(-c5ccccc5)cc(-c5ccccc5)c4)nc(-c4cc(-c5ccc6ccccc6c5)cc5oc6ccccc6c45)n3)cc2)cc1]

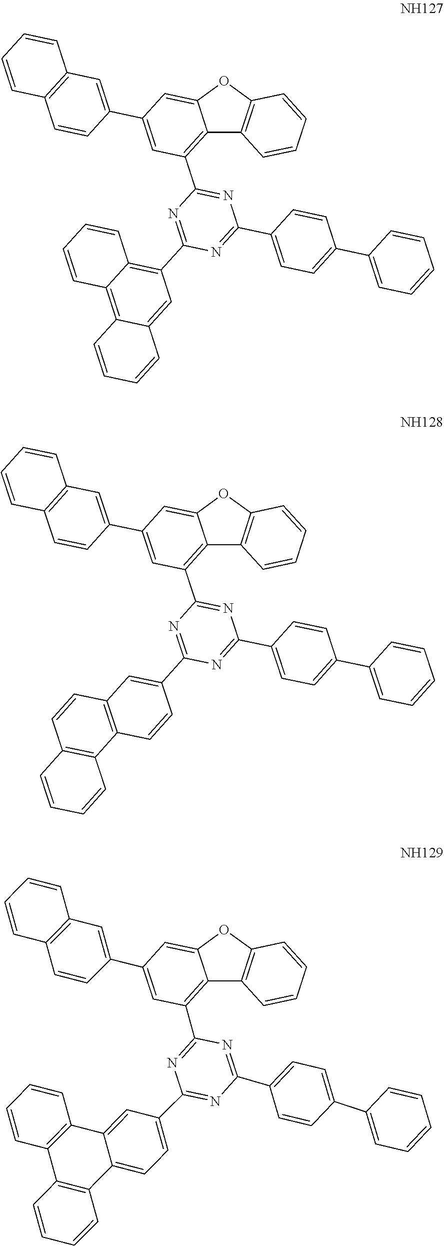 [SMILES: c1ccc(-c2ccc(-c3nc(-c4cc5ccccc5c5ccccc45)nc(-c4cc(-c5ccc6ccccc6c5)cc5oc6ccccc6c45)n3)cc2)cc1 ; c1ccc(-c2ccc(-c3nc(-c4ccc5c(ccc6ccccc65)c4)nc(-c4cc(-c5ccc6ccccc6c5)cc5oc6ccccc6c45)n3)cc2)cc1 ; c1ccc(-c2ccc(-c3nc(-c4ccc5c6ccccc6c6ccccc6c5c4)nc(-c4cc(-c5ccc6ccccc6c5)cc5oc6ccccc6c45)n3)cc2)cc1]

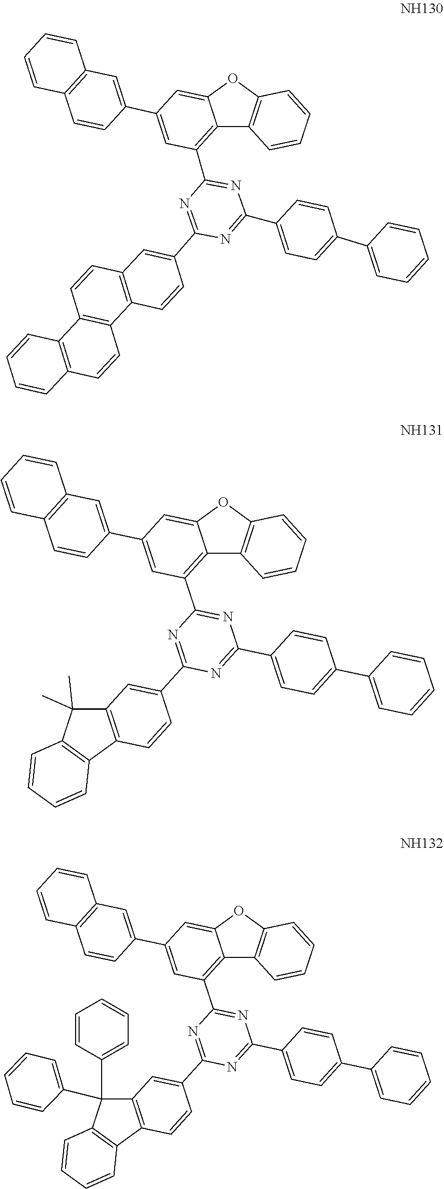 [SMILES: c1ccc(-c2ccc(-c3nc(-c4ccc5c(ccc6c7ccccc7ccc56)c4)nc(-c4cc(-c5ccc6ccccc6c5)cc5oc6ccccc6c45)n3)cc2)cc1 ; CC1(C)c2ccccc2-c2ccc(-c3nc(-c4ccc(-c5ccccc5)cc4)nc(-c4cc(-c5ccc6ccccc6c5)cc5oc6ccccc6c45)n3)cc21 ; c1ccc(-c2ccc(-c3nc(-c4ccc5c(c4)C(c4ccccc4)(c4ccccc4)c4ccccc4-5)nc(-c4cc(-c5ccc6ccccc6c5)cc5oc6ccccc6c45)n3)cc2)cc1]

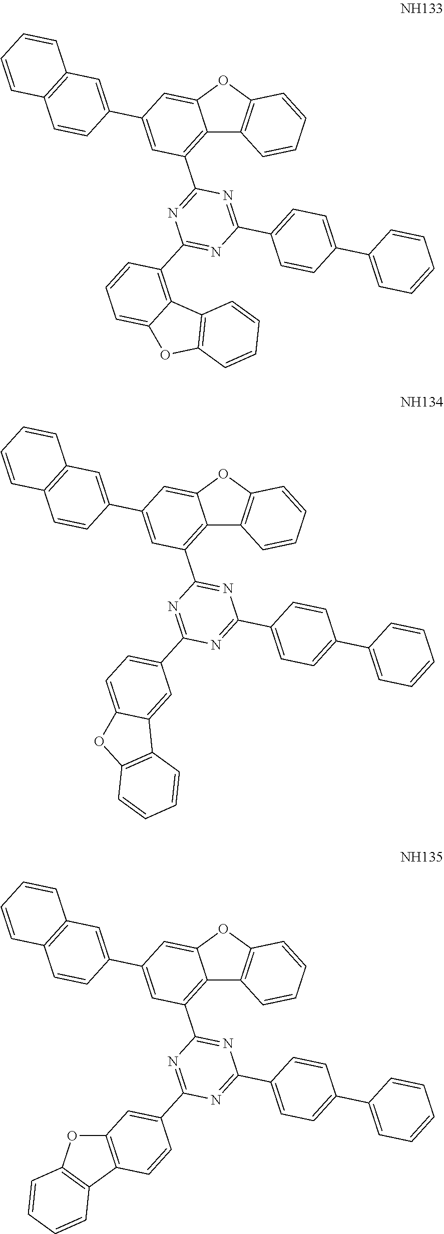 [SMILES: c1ccc(-c2ccc(-c3nc(-c4cccc5oc6ccccc6c45)nc(-c4cc(-c5ccc6ccccc6c5)cc5oc6ccccc6c45)n3)cc2)cc1 ; c1ccc(-c2ccc(-c3nc(-c4ccc5oc6ccccc6c5c4)nc(-c4cc(-c5ccc6ccccc6c5)cc5oc6ccccc6c45)n3)cc2)cc1 ; c1ccc(-c2ccc(-c3nc(-c4ccc5c(c4)oc4ccccc45)nc(-c4cc(-c5ccc6ccccc6c5)cc5oc6ccccc6c45)n3)cc2)cc1]

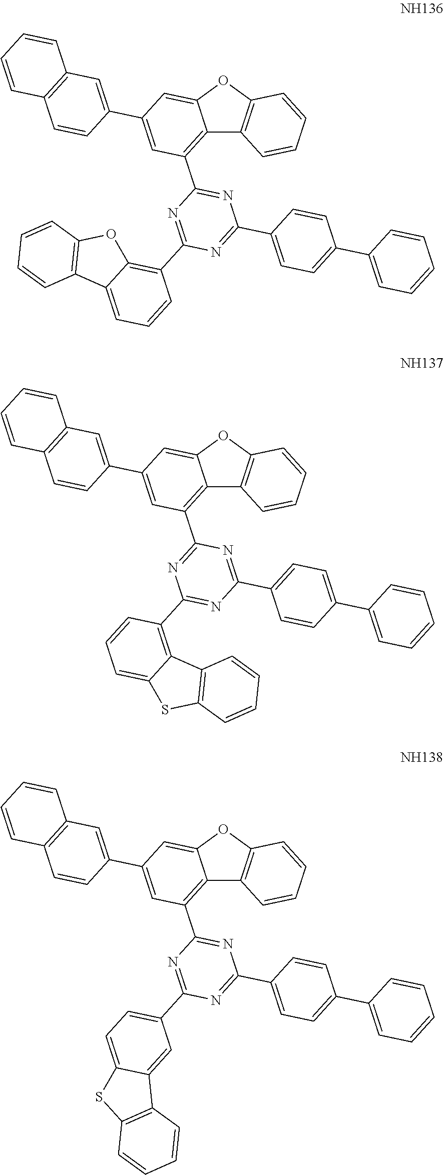 [SMILES: c1ccc(-c2ccc(-c3nc(-c4cccc5c4oc4ccccc45)nc(-c4cc(-c5ccc6ccccc6c5)cc5oc6ccccc6c45)n3)cc2)cc1 ; c1ccc(-c2ccc(-c3nc(-c4cc(-c5ccc6ccccc6c5)cc5oc6ccccc6c45)nc(-c4cccc5sc6ccccc6c45)n3)cc2)cc1 ; c1ccc(-c2ccc(-c3nc(-c4ccc5sc6ccccc6c5c4)nc(-c4cc(-c5ccc6ccccc6c5)cc5oc6ccccc6c45)n3)cc2)cc1]

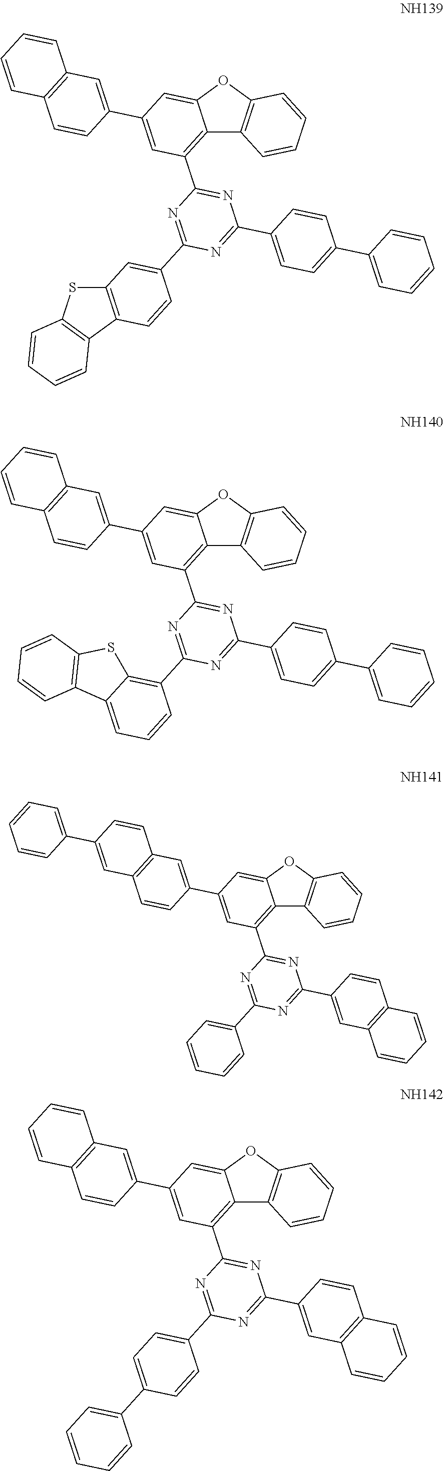 [SMILES: c1ccc(-c2ccc(-c3nc(-c4ccc5c(c4)sc4ccccc45)nc(-c4cc(-c5ccc6ccccc6c5)cc5oc6ccccc6c45)n3)cc2)cc1 ; c1ccc(-c2ccc(-c3nc(-c4cccc5c4sc4ccccc45)nc(-c4cc(-c5ccc6ccccc6c5)cc5oc6ccccc6c45)n3)cc2)cc1 ; c1ccc(-c2ccc3cc(-c4cc(-c5nc(-c6ccccc6)nc(-c6ccc7ccccc7c6)n5)c5c(c4)oc4ccccc45)ccc3c2)cc1 ; c1ccc(-c2ccc(-c3nc(-c4ccc5ccccc5c4)nc(-c4cc(-c5ccc6ccccc6c5)cc5oc6ccccc6c45)n3)cc2)cc1]

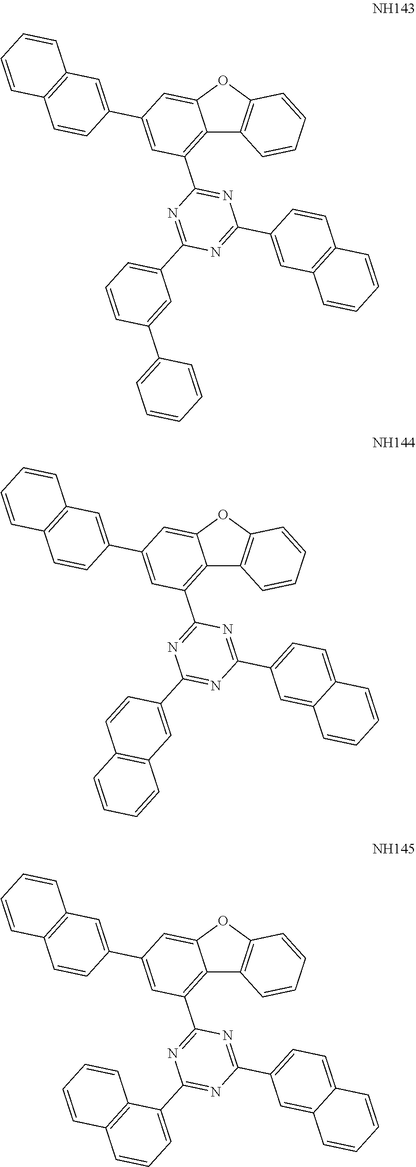 [SMILES: c1ccc(-c2cccc(-c3nc(-c4ccc5ccccc5c4)nc(-c4cc(-c5ccc6ccccc6c5)cc5oc6ccccc6c45)n3)c2)cc1 ; c1ccc2cc(-c3cc(-c4nc(-c5ccc6ccccc6c5)nc(-c5ccc6ccccc6c5)n4)c4c(c3)oc3ccccc34)ccc2c1 ; c1ccc2cc(-c3cc(-c4nc(-c5ccc6ccccc6c5)nc(-c5cccc6ccccc56)n4)c4c(c3)oc3ccccc34)ccc2c1]

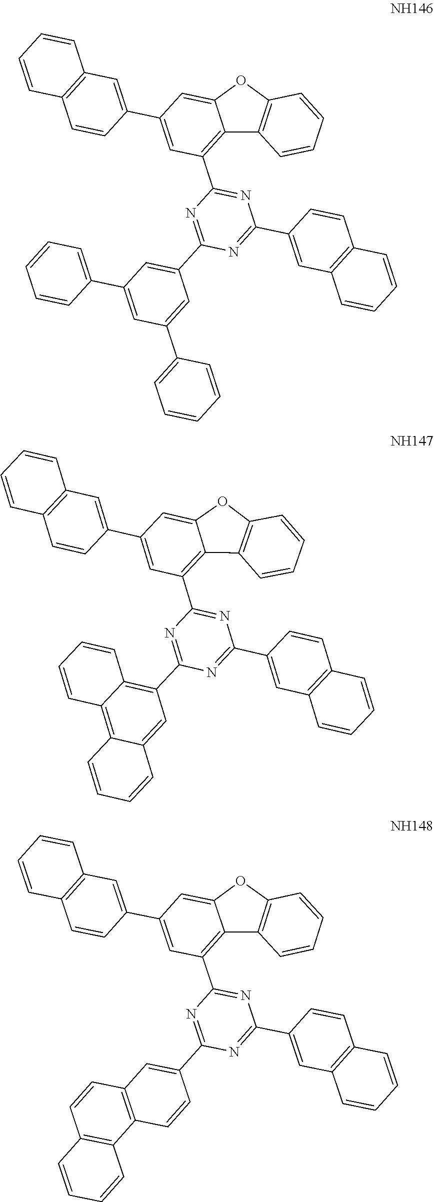 [SMILES: c1ccc(-c2cc(-c3ccccc3)cc(-c3nc(-c4ccc5ccccc5c4)nc(-c4cc(-c5ccc6ccccc6c5)cc5oc6ccccc6c45)n3)c2)cc1 ; c1ccc2cc(-c3cc(-c4nc(-c5ccc6ccccc6c5)nc(-c5cc6ccccc6c6ccccc56)n4)c4c(c3)oc3ccccc34)ccc2c1 ; c1ccc2cc(-c3cc(-c4nc(-c5ccc6ccccc6c5)nc(-c5ccc6c(ccc7ccccc76)c5)n4)c4c(c3)oc3ccccc34)ccc2c1]

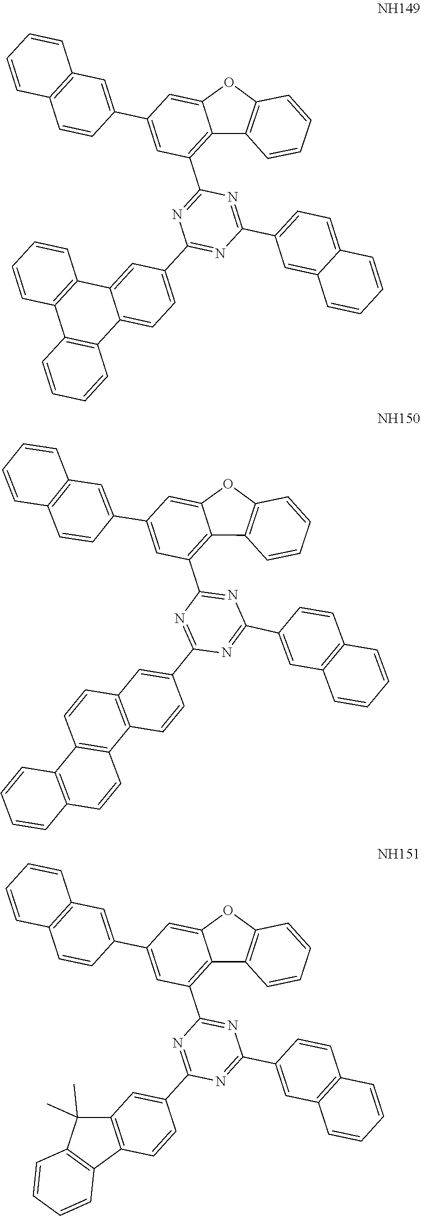

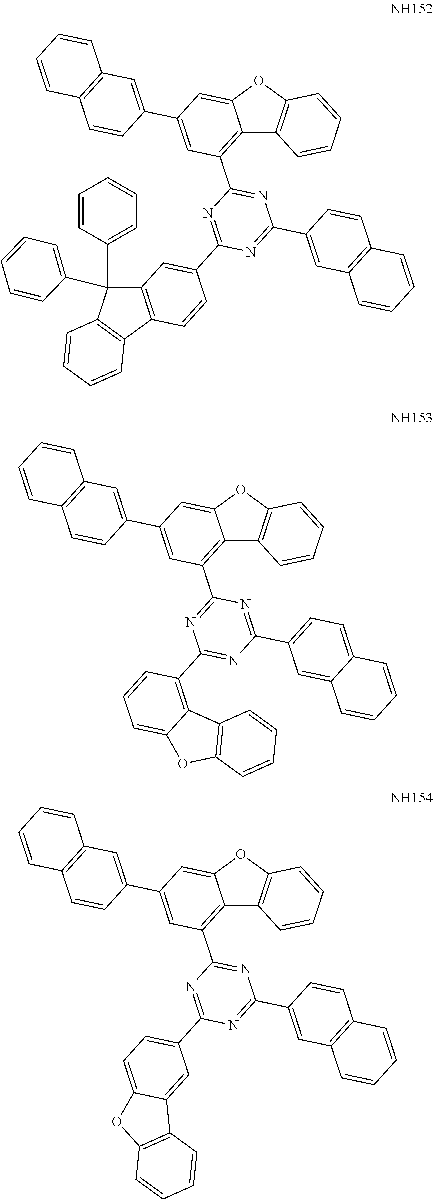 [SMILES: c1ccc(C2(c3ccccc3)c3ccccc3-c3ccc(-c4nc(-c5ccc6ccccc6c5)nc(-c5cc(-c6ccc7ccccc7c6)cc6oc7ccccc7c56)n4)cc32)cc1 ; c1ccc2cc(-c3cc(-c4nc(-c5ccc6ccccc6c5)nc(-c5cccc6oc7ccccc7c56)n4)c4c(c3)oc3ccccc34)ccc2c1 ; c1ccc2cc(-c3cc(-c4nc(-c5ccc6ccccc6c5)nc(-c5ccc6oc7ccccc7c6c5)n4)c4c(c3)oc3ccccc34)ccc2c1]

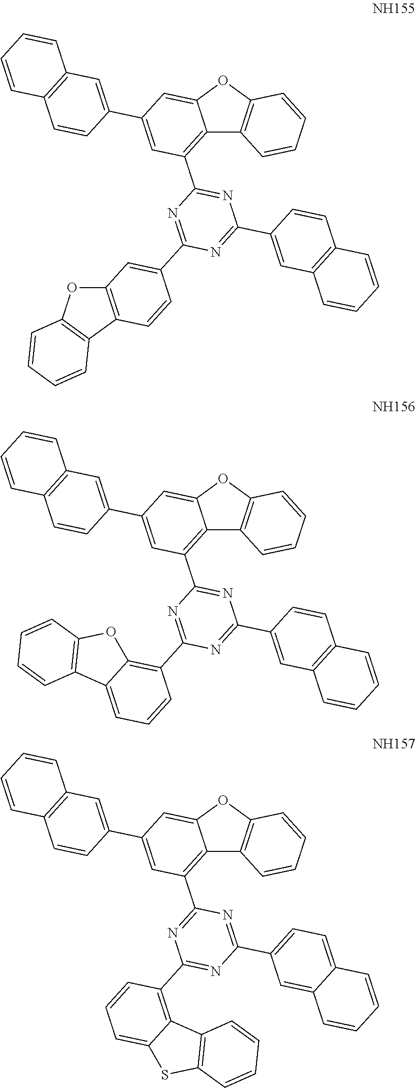 [SMILES: c1ccc2cc(-c3cc(-c4nc(-c5ccc6ccccc6c5)nc(-c5ccc6c(c5)oc5ccccc56)n4)c4c(c3)oc3ccccc34)ccc2c1 ; c1ccc2cc(-c3cc(-c4nc(-c5ccc6ccccc6c5)nc(-c5cccc6c5oc5ccccc56)n4)c4c(c3)oc3ccccc34)ccc2c1 ; c1ccc2cc(-c3cc(-c4nc(-c5ccc6ccccc6c5)nc(-c5cccc6sc7ccccc7c56)n4)c4c(c3)oc3ccccc34)ccc2c1]

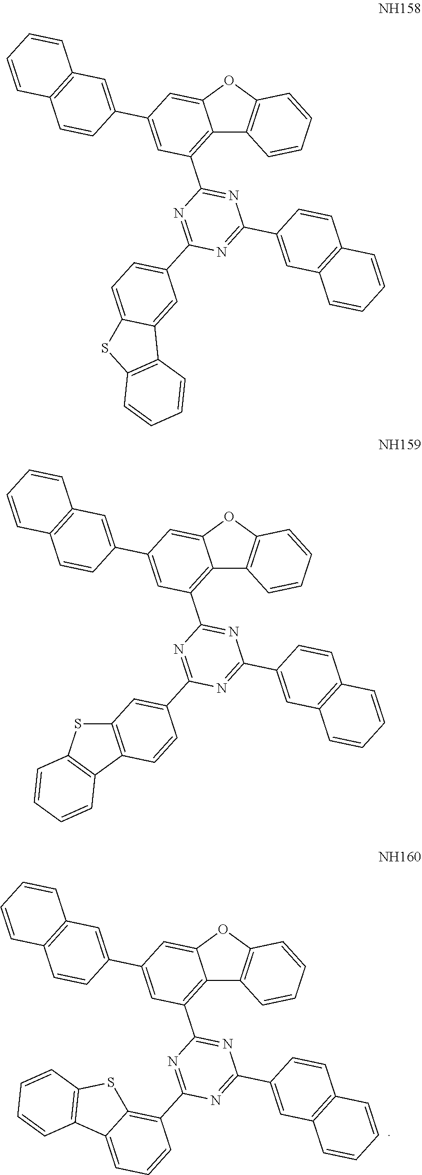 [SMILES: c1ccc2cc(-c3cc(-c4nc(-c5ccc6ccccc6c5)nc(-c5ccc6sc7ccccc7c6c5)n4)c4c(c3)oc3ccccc34)ccc2c1 ; c1ccc2cc(-c3cc(-c4nc(-c5ccc6ccccc6c5)nc(-c5ccc6c(c5)sc5ccccc56)n4)c4c(c3)oc3ccccc34)ccc2c1 ; c1ccc2cc(-c3cc(-c4nc(-c5ccc6ccccc6c5)nc(-c5cccc6c5sc5ccccc56)n4)c4c(c3)oc3ccccc34)ccc2c1]

14. A composition for forming an organic material layer, comprising the heterocyclic compound of claim 1.

15. The composition of claim 14, further comprising a compound of the following Chemical Formula 2: 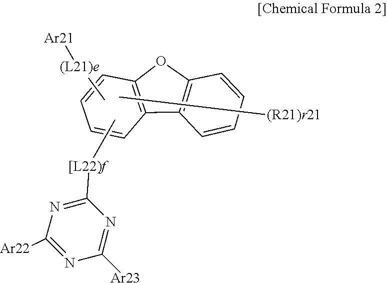
in Chemical Formula 2,
L21 and L22 are each independently a direct bond; or a substituted or unsubstituted C6 to C60 arylene group,
e and f are an integer from 0 to 4,
when e and f are 2 or higher, substituents in the parenthesis are the same as or different from each other,
Ar21 is a substituted or unsubstituted C6 to C60 aryl group,
Ar22 and Ar23 are each independently a substituted or unsubstituted C6 to C60 aryl group; or a substituted or unsubstituted C2 to C60 heteroaryl group,
R21 is hydrogen; deuterium; a halogen group; a cyano group; a substituted or unsubstituted C1 to C60 alkyl group; a substituted or unsubstituted C3 to C60 cycloalkyl group; a substituted or unsubstituted C2 to C60 heterocycloalkyl group; a substituted or unsubstituted C6 to C60 aryl group; or a substituted or unsubstituted C2 to C60 heteroaryl group, and
r21 is an integer from 0 to 6, and when r21 is 2 or higher, R21's are the same as or different from each other.

16. The composition of claim 15, wherein a weight ratio of the heterocyclic compound and the compound of Chemical Formula 2 is 1:10 to 10:1.
